(19) United States
(12) Patent Application Publication
(10) Pub. No.: US 2023/0114241 A1
(43) Pub. Date: Apr. 13, 2023

(54) METHODS OF TREATING EPILEPSY USING THE SAME

(71) Applicant: Trevena, Inc., Chesterbrook, PA (US)

(72) Inventors: Mark A. DEMITRACK, Chesterbrook, PA (US); Michael S. KRAMER, Chesterbrook, PA (US)

(21) Appl. No.: 17/639,401

(22) PCT Filed: Sep. 3, 2020
(86) PCT No.: PCT/US20/49147

§ 371 Date: Mar. 1, 2022

Related U.S. Application Data
(60) Provisional application No. 62/896,116, filed on Sep. 5, 2019.

(51) Int. Cl. A61K 31/4245; A61K 31/4439; A61K 31/497; A61K 31/433; A61K 31/427; A61K 31/4709; A61K 31/437; A61K 31/444; A61K 31/4192; A61K 31/4725; A61K 31/506; A61K 31/498; A61P 25/08

(52) U.S. Cl. CPC A61K 31/4245; A61K 31/4439; A61K 31/497; A61K 31/433; A61K 31/427; A61K 31/4709; A61K 31/437; A61K 31/444; A61K 31/4192; A61K 31/4725; A61K 31/506; A61K 31/498; A61P 25/08

(57) ABSTRACT

The present embodiments are directed, in part, to compounds, or pharmaceutically acceptable salts thereof, or pharmaceutical compositions thereof for modulating the activity of S1P1 receptor and methods of using the same for the treatment of seizures, epilepsy related conditions, epilepsy-related syndrome, and the like as described herein.

### CROSS-REFERENCE TO RELATED APPLICATIONS

The present application claims priority to U.S. Provisional Application No. 62/896,116, filed Sep. 5, 2019, which is hereby incorporated by reference in its entirety.

### FIELD

Embodiments disclosed herein are directed, in part, to compounds, or pharmaceutically acceptable salts thereof, for modulating S1P1 receptor activity and/or methods for treating and/or preventing epilepsy, epilepsy-related syndrome, and the like as described herein.

### BACKGROUND

Epilepsy is a chronic neurological disorder presenting a wide spectrum of diseases that affect 2.2 million people in the United States and >65 million people worldwide (Hirtz et al. 2007). Currently available antiepileptic drugs suffer from a range of side effects and there is a significant group of patients comprising about 20-30% of cases that are resistant to the currently available therapeutic agents. Thus, there is a need for new compounds and compositions for treating and/or preventing epilepsy. The compounds and compositions described herein fulfill these needs as well as others.

### SUMMARY OF EMBODIMENTS

Provided are methods of treating or preventing a seizure, an epilepsy or an epilepsy-related syndrome, and the like as described herein in a subject as described herein. In some embodiments, the methods comprise administering to the subject a compound or a pharmaceutically acceptable salt thereof or a pharmaceutical composition as described herein.

In some embodiments, the compounds described herein, in part, modulate the activity of the S1P1 receptor. In some embodiments, the methods comprise administering one or more compounds described herein to a subject.

In some embodiments, provided are methods of treating or preventing a seizure, an epilepsy or an epilepsy-related syndrome, and the like as described herein, in a subject, wherein the methods comprise administering to the subject a compound having a formula of Formula I or Formula II:

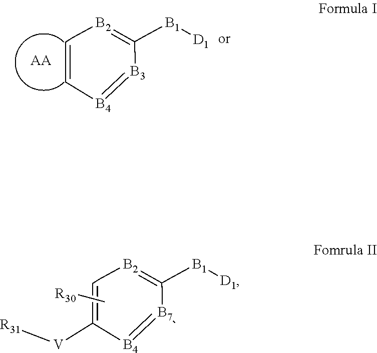

or, or a pharmaceutically acceptable salt thereof, wherein AA, B₁, B₂, B₃, B₄, D₁, V, R₃₀, and R₃₁ are as provided for herein and, for example, can be selected from the respective groups of chemical moieties described herein. Also provided are processes for preparing these compounds.

In some embodiments, also provided are methods of treating or preventing a seizure, an epilepsy or an epilepsy-related syndrome, and the like as described herein, in a subject, wherein the methods comprise administering to the subject pharmaceutical compositions comprising one or more compounds as described herein, which can also comprise a pharmaceutically acceptable carrier. In some embodiments, the compounds described herein can be provided in any form, such as a solid or solution (e.g., aqueous solution), such as is described herein. The compounds described herein, for example, can be obtained and employed in lyophilized form alone or with suitable additives.

### DESCRIPTION OF EMBODIMENTS

Unless defined otherwise, all technical and scientific terms have the same meaning as is commonly understood by one of ordinary skill in the art to which the embodiments disclosed belongs.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “acylamino” means an amino group substituted by an acyl group (e.g., —O—C(═O)—H or —O—C(═O)-alkyl). An example of an acylamino is —NHC(═O)H or —NHC(═O)CH₃. The term “lower acylamino” refers to an amino group substituted by a lower acyl group (e.g., —O—C(═O)—H or —O—C(═O)—C₁₋₆alkyl). An example of a lower acylamino is —NHC(═O)H or —NHC(═O)CH₃.

As used herein, the term “alkenyl” means a straight or branched alkyl group having one or more double carbon-carbon bonds and 2-20 carbon atoms, including, but not limited to, ethenyl, 1-propenyl, 2-propenyl, 2-methyl-1-propenyl, 1-butenyl, 2-butenyl, and the like. In some embodiments, the alkenyl chain is from 2 to 10 carbon atoms in length, from 2 to 8 carbon atoms in length, from 2 to 6 carbon atoms in length, or from 2 to 4 carbon atoms in length.

As used herein, the term “anti-epileptic drug(s)” (also commonly known as anticonvulsants or anti-seizure drugs) or “AED(s)” generally encompasses pharmacological agents that reduce the frequency or likelihood of a seizure. There are many drug classes that comprise the set of antiepileptic drugs (AEDs), and many different mechanisms of action are represented. For example, some medications are believed to increase the seizure threshold, thereby making the brain less likely to initiate a seizure. Other medications retard the spread of neural bursting activity and tend to prevent the propagation or spread of seizure activity. Some AEDs, such as the Benzodiazepines, act via the GABA receptor and globally suppress neural activity. However, other AEDs may act by modulating a neuronal calcium channel, a neuronal potassium channel, a neuronal NMDA channel, a neuronal AMPA channel, a neuronal metabotropic type channel, a neuronal sodium channel, and/or a neuronal kainite channel. The phrase “Anti-epileptic drugs that block sodium channels”, “sodium-channel-blocking AEDs” used herein refers to anti-epileptic drugs that block sodium channels. The sodium-channel-blocking AEDs can be selected from the group consisting of carbamazepine, clonazepam, eslicarbazepine, ethosuximide, felbamate, gabapentin, lacosamide, lamotrigine, levetiracetam, oxcarbazepine, phenobarbital, phenytoin, pregabalin, primidone, rufnamide, tiagabine, topiramate, vigabatrin, valproate (valproic acid), and zonisamide and, as well as other existing or new AEDs which may be identified to block sodium channels in the future.

The terms “alkoxy”, “phenyloxy”, “benzoxy” and “pyrimidinyloxy” refer to an alkyl group, phenyl group, benzyl group, or pyrimidinyl group, respectively, each optionally substituted, that is bonded through an oxygen atom. For example, the term “alkoxy” means a straight or branched —O-alkyl group of 1 to 20 carbon atoms, including, but not limited to, methoxy, ethoxy, n-propoxy, isopropoxy, t-butoxy, and the like. In some embodiments, the alkoxy chain is from 1 to 10 carbon atoms in length, from 1 to 8 carbon atoms in length, from 1 to 6 carbon atoms in length, from 1 to 4 carbon atoms in length, from 2 to 10 carbon atoms in length, from 2 to 8 carbon atoms in length, from 2 to 6 carbon atoms in length, or from 2 to 4 carbon atoms in length.

As used herein, the term “alkyl” means a saturated hydrocarbon group, which is straight-chained or branched. An alkyl group can contain from 1 to 20, from 2 to 20, from 1 to 10, from 2 to 10, from 1 to 8, from 2 to 8, from 1 to 6, from 2 to 6, from 1 to 4, from 2 to 4, from 1 to 3, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, 2,2,4-trimethylpentyl, nonyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the term “allylamino” means an amino group substituted by an alkyl group having from 1 to 6 carbon atoms. An example of an alkylamino is —NHCH₂CH₃.

As used herein, the term “alkylene” or “alkylenyl” means a divalent alkyl linking group.

An example of an alkylene (or alkylenyl) is methylene or methylenyl (—CH₂—).

As used herein, the term “alkylthio” means an —S-alkyl group having from 1 to 6 carbon atoms. An example of an alkylthio group is —SCH₂CH₃.

As used herein, the term “alkynyl” means a straight or branched alkyl group having one or more triple carbon-carbon bonds and 2-20 carbon atoms, including, but not limited to, acetylene, 1-propylene, 2-propylene, and the like. In some embodiments, the alkynyl chain is 2 to 10 carbon atoms in length, from 2 to 8 carbon atoms in length, from 2 to 6 carbon atoms in length, or from 2 to 4 carbon atoms in length.

As used herein, the term “amidino” means —C(═NH)NH₂.

As used herein, the term “amino” means —NH₂.

As used herein, the term “aminoalkoxy” means an alkoxy group substituted by an amino group. An example of an aminoalkoxy is —OCH₂CH₂NH₂.

As used herein, the term “aminoalkyl” means an alkyl group substituted by an amino group. An example of an aminoalkyl is —CH₂CH₂NH₂.

As used herein, the term “aminosulfonyl” means —S(═O)₂NH₂.

As used herein, the term “aminoalkylthio” means an alkylthio group substituted by an amino group. An example of an aminoalkylthio is —SCH₂CH₂NH₂.

As used herein, the term “amphiphilic” means a three-dimensional structure having discrete hydrophobic and hydrophilic regions. An amphiphilic compound suitably has the presence of both hydrophobic and hydrophilic elements.

As used herein, the term “animal” includes, but is not limited to, humans and non-human vertebrates such as wild, domestic, and farm animals.

As used herein, the term “antagonize” or “antagonizing” means reducing or completely eliminating an effect, such as an activity of the S1P1 receptor.

As used herein, the phrase “anti-receptor effective amount” of a compound can be measured by the anti-receptor effectiveness of the compound. In some embodiments, an anti-receptor effective amount inhibits an activity of the receptor by at least 10%, by at least 20%, by at least 30%, by at least 40%, by at least 50%, by at least 60%, by at least 70%, by at least 80%, by at least 90%, or by at least 95%. In some embodiments, an “anti-receptor effective amount” is also a “therapeutically effective amount” whereby the compound reduces or eliminates or modulates at least one effect of a S1P1 receptor. In some embodiments, the effect is the beta-arrestin effect. In some embodiments, the effect is the G-protein mediated effect.

As used herein, the term “aryl” means a monocyclic, bicyclic, or polycyclic (e.g., having 2, 3 or 4 fused rings) aromatic hydrocarbons. In some embodiments, aryl groups have from 6 to 20 carbon atoms or from 6 to 10 carbon atoms. Examples of aryl groups include, but are not limited to, phenyl, naphthyl, anthracenyl, phenanthrenyl, indanyl, indenyl, tetrahydronaphthyl, and the like. Examples of aryl groups include, but are not limited to:

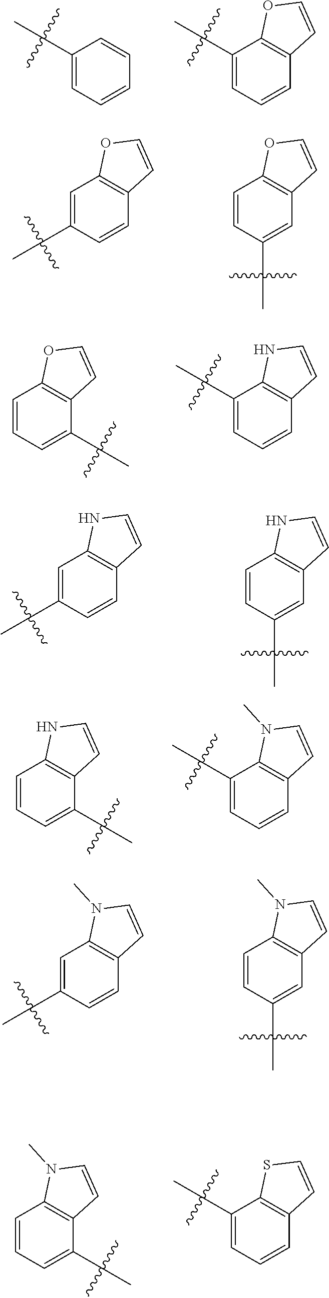

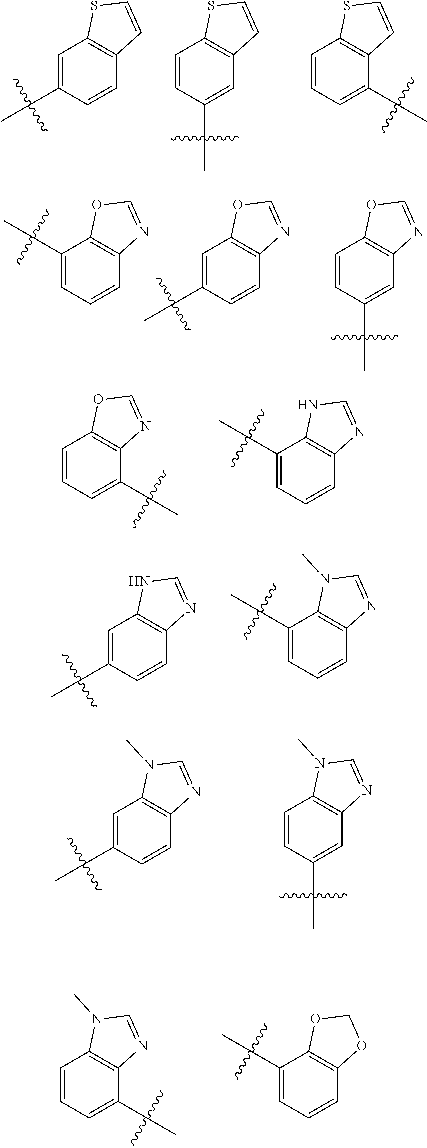

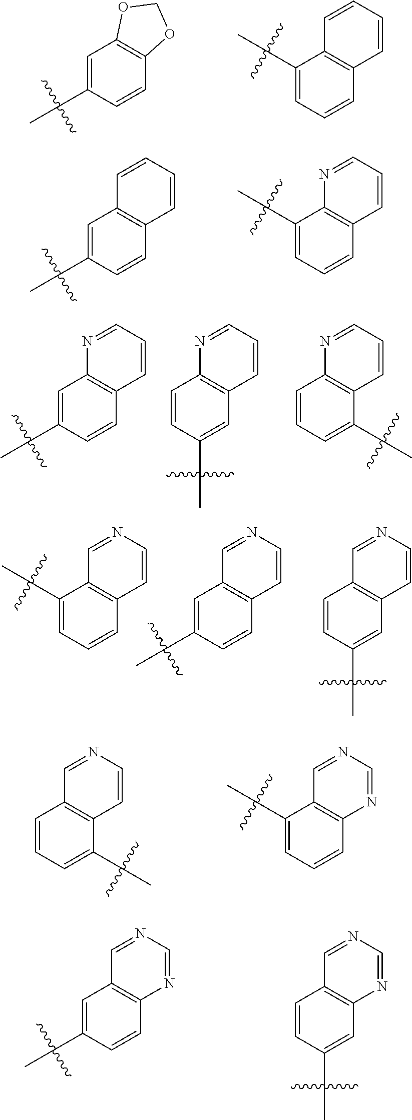

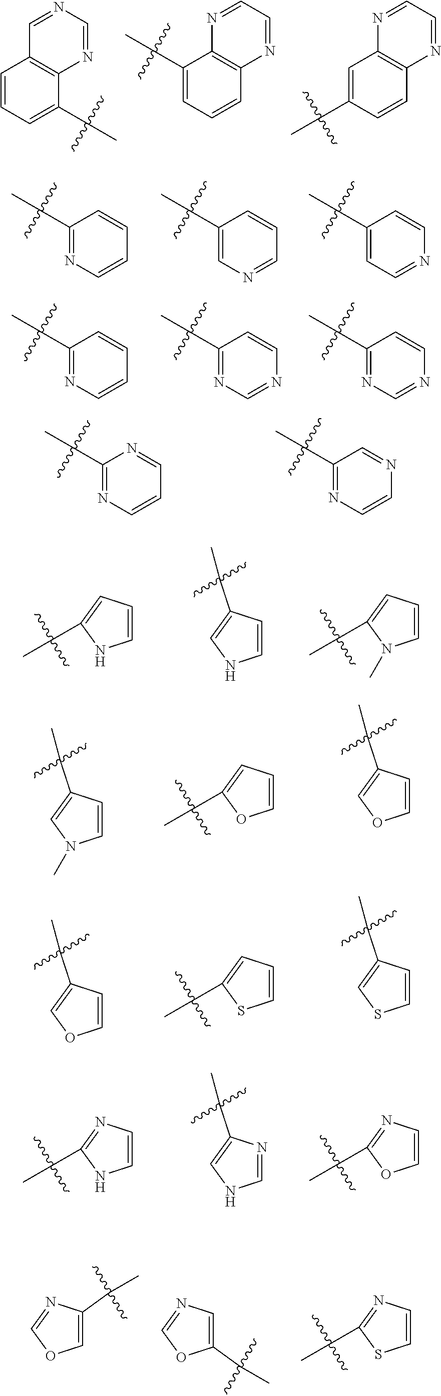

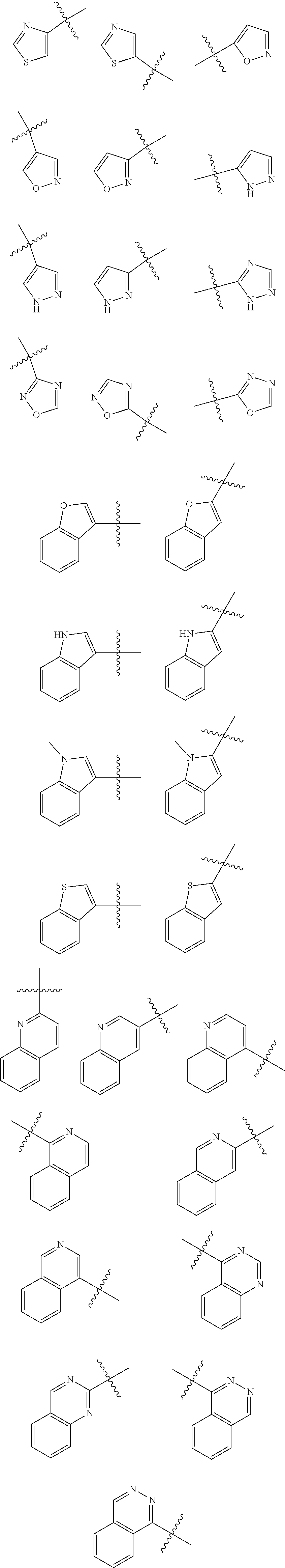

As used herein, the term “arylalkyl” means a C₁₋₆alkyl substituted by aryl.

As used herein, the term “arylamino” means an amino group substituted by an aryl group.

An example of an arylamino is —NH(phenyl).

As used herein, the term “arylene” means an aryl linking group, i.e., an aryl group that links one group to another group in a molecule.

As used herein, the term “carbamoyl” means —C(═O)—NH₂.

As used herein, the term “carbocycle” means a 5- or 6-membered, saturated or unsaturated cyclic ring, optionally containing O, S, or N atoms as part of the ring. Examples of carbocycles include, but are not limited to, cyclopentyl, cyclohexyl, cyclopenta-1,3-diene, phenyl, and any of the heterocycles recited above.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, the term, “compound” means all stereoisomers, tautomers, and isotopes of the compounds described herein.

As used herein, the term “complex partial seizure” means one of the symptoms associated with intractable epilepsy, refers to a partial seizure with impairment of consciousness, and is similar to a seizure that has conventionally been called a psycho-motor seizure or a seizure associated with temporal lobe epilepsy. In the international classification draft (1981), the complex partial seizure is defined as a seizure with impairment of consciousness exhibiting an electroencephalogram during a seizure in which unilateral or bilateral electric discharge attributed to a focus in a diffuse or a temporal or front-temporal portion.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together of two elements in an in vitro system or an in vivo system. For example, “contacting” a S1P1 receptor compound with a S1P1 receptor in an individual, or patient, or cell, includes the administration of the compound to an individual or patient, such as a human, as well as, for example, introducing a compound into a sample containing a cellular or purified preparation containing the S1P1 receptor.

As used herein, the term “cortex epilepsy” means one type of intractable epilepsy, is an epilepsy having a focus in the cerebral cortex, and is classified as symptomatic epilepsy belonging to localization-related (focal) epilepsies and syndromes in the international classification of epilepsy. In the international classification, seizures associated with cortex epilepsy are classified as simple partial seizures, which are partial seizures without reduction of consciousness. Accordingly, an electroencephalogram taken during a seizure associated with cortex epilepsy (not always recorded on the scalp) exhibits localized contralateral electric discharge from the corresponding cortical field. The cortex epilepsy is classified as temporal lobe epilepsy, parietal lobe epilepsy, or occipital lobe epilepsy.

As used herein, the term “cyano” means —CN.

As used herein, the term “cycloalkyl” means non-aromatic cyclic hydrocarbons including cyclized alkyl, alkenyl, and alkynyl groups that contain up to 20 ring-forming carbon atoms.

Cycloalkyl groups can include mono- or polycyclic ring systems such as fused ring systems, bridged ring systems, and spiro ring systems. In some embodiments, polycyclic ring systems include 2, 3, or 4 fused rings. A cycloalkyl group can contain from 3 to 15, from 3 to 10, from 3 to 8, from 3 to 6, from 4 to 6, from 3 to 5, or 5 or 6 ring-forming carbon atoms. Ring-forming carbon atoms of a cycloalkyl group can be optionally substituted by oxo or sulfido. Examples of cycloalkyl groups include, but are not limited to, cyclopropyl, cyclobutyl, cyclopentyl, cyclohexyl, cycloheptyl, cyclooctyl, cyclononyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (having a bond in common with) to the cycloalkyl ring, for example, benzo or thienyl derivatives of pentane, pentene, hexane, and the like (e.g., 2,3-dihydro-1H-indene-1-yl, or 1H-inden-2(3H)-one-1-yl).

As used herein, the term “cycloalkylalkyl” means a C₁₋₆alkyl substituted by cycloalkyl.

As used herein, the term “dialkylamino” means an amino group substituted by two alkyl groups, each having from 1 to 6 carbon atoms.

As used herein, the term “diazamino” means —N(NH₂)₂.

As used herein, the terms “epilepsy”, “epileptic seizures” and “epileptic syndromes” are meant to include all known types of epileptic seizures and syndromes including; partial seizures, including simple, complex and partial seizures evolving to generalized tonic-clonic convulsions and generalized seizures, both convulsive and nonconvulsive and unclassified epileptic seizures.

As used herein, the term “facially amphiphilic” or “facial amphiphilicity” means compounds with polar (hydrophilic) and nonpolar (hydrophobic) side chains that adopt conformation(s) leading to segregation of polar and nonpolar side chains to opposite faces or separate regions of the structure or molecule.

As used herein, the term “guanidino” means —NH(═NH)NH₂.

As used herein, the term “halo” means halogen groups including, but not limited to fluoro, chloro, bromo, and iodo.

As used herein, the term “haloalkoxy” means an —O-haloalkyl group. An example of an haloalkoxy group is OCF₃.

As used herein, the term “haloalkyl” means a C₁₋₆alkyl group having one or more halogen substituents. Examples of haloalkyl groups include, but are not limited to, CF₃, C₂F₅, CH₂F, CHF₂, CCl₃, CHCl₂, CH₂CF₃, and the like.

As used herein, the term “heteroaryl” means an aromatic heterocycle having up to 20 ring-forming atoms (e.g., C) and having at least one heteroatom ring member (ring-forming atom) such as sulfur, oxygen, or nitrogen. In some embodiments, the heteroaryl group has at least one or more heteroatom ring-forming atoms, each of which are, independently, sulfur, oxygen, or nitrogen. In some embodiments, the heteroaryl group has from 3 to 20 ring-forming atoms, from 3 to 10 ring-forming atoms, from 3 to 6 ring-forming atoms, or from 3 to 5 ring-forming atoms. In some embodiments, the heteroaryl group contains 2 to 14 carbon atoms, from 2 to 7 carbon atoms, or 5 or 6 carbon atoms. In some embodiments, the heteroaryl group has 1 to 4 heteroatoms, 1 to 3 heteroatoms, or 1 or 2 heteroatoms. Heteroaryl groups include monocyclic and polycyclic (e.g., having 2, 3 or 4 fused rings) systems. Examples of heteroaryl groups include, but are not limited to, pyridyl, pyrimidinyl, pyrazinyl, pyridazinyl, triazinyl, furyl, quinolyl, isoquinolyl, thienyl, imidazolyl, thiazolyl, indolyl (such as indol-3-yl), pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, pyranyl, oxadiazolyl, isoxazolyl, triazolyl, thianthrenyl, pyrazolyl, indolizinyl, isoindolyl, isobenzofuranyl, benzoxazolyl, xanthenyl, 2H-pyrrolyl, pyrrolyl, 3H-indolyl, 4H-quinolizinyl, phthalazinyl, naphthyridinyl, quinazolinyl, phenanthridinyl, acridinyl, perimidinyl, phenanthrolinyl, phenazinyl, isothiazolyl, phenothiazinyl, isoxazolyl, furanyl, phenoxazinyl groups, and the like. Suitable heteroaryl groups include 1,2,3-triazole, 1,2,4-triazole, 5-amino-1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, and 2-aminopyridine.

As used herein, the term “heteroarylalkyl” means a C₁₋₆alkyl group substituted by a heteroaryl group.

As used herein, the term “heteroarylamino” means an amino group substituted by a heteroaryl group. An example of a heteroarylamino is —NH-(2-pyridyl).

As used herein, the term “heteroarylene” means a heteroaryl linking group, i.e., a heteroaryl group that links one group to another group in a molecule.

As used herein, the term “heterocycle” or “heterocyclic ring” means a 5- to 7-membered mono- or bicyclic or 7- to 10-membered bicyclic heterocyclic ring system any ring of which may be saturated or unsaturated, and which consists of carbon atoms and from one to three heteroatoms chosen from N, O and S, and wherein the N and S heteroatoms may optionally be oxidized, and the N heteroatom may optionally be quaternized, and including any bicyclic group in which any of the above-defined heterocyclic rings is fused to a benzene ring. Particularly useful are rings containing one oxygen or sulfur, one to three nitrogen atoms, or one oxygen or sulfur combined with one or two nitrogen atoms. The heterocyclic ring may be attached at any heteroatom or carbon atom which results in the creation of a stable structure. Examples of heterocyclic groups include, but are not limited to, piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

As used herein, the term “heterocycloalkyl” means non-aromatic heterocycles having up to 20 ring-forming atoms including cyclized alkyl, alkenyl, and alkynyl groups, where one or more of the ring-forming carbon atoms is replaced by a heteroatom such as an O, N, or S atom. Hetercycloalkyl groups can be mono or polycyclic (e.g., fused, bridged, or spiro systems). In some embodiments, the heterocycloalkyl group has from 1 to 20 carbon atoms, or from 3 to 20 carbon atoms. In some embodiments, the heterocycloalkyl group contains 3 to 14 ring-forming atoms, 3 to 7 ring-forming atoms, or 5 or 6 ring-forming atoms. In some embodiments, the heterocycloalkyl group has 1 to 4 heteroatoms, 1 to 3 heteroatoms, or 1 or 2 heteroatoms. In some embodiments, the heterocycloalkyl group contains 0 to 3 double bonds. In some embodiments, the heterocycloalkyl group contains 0 to 2 triple bonds. Examples of heterocycloalkyl groups include, but are not limited to, morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, oxazolidinyl, isothiazolidinyl, pyrazolidinyl, thiazolidinyl, imidazolidinyl, pyrrolidin-2-one-3-yl, and the like. In addition, ring-forming carbon atoms and heteroatoms of a heterocycloalkyl group can be optionally substituted by oxo or sulfido. For example, a ring-forming S atom can be substituted by 1 or 2 oxo (form a S(O) or S(O)₂). For another example, a ring-forming C atom can be substituted by oxo (form carbonyl). Also included in the definition of heterocycloalkyl are moieties that have one or more aromatic rings fused (having a bond in common with) to the nonaromatic heterocyclic ring including, but not limited to, pyridinyl, thiophenyl, phthalimidyl, naphthalimidyl, and benzo derivatives of heterocycles such as indolene, isoindolene, 4,5,6,7-tetrahydrothieno[2,3-c]pyridine-5-yl, 5,6-dihydrothieno[2,3-c]pyridin-7(4H)-one-5-yl, isoindolin-1-one-3-yl, and 3,4-dihydroisoquinolin-1(2H)-one-3yl groups. Ring-forming carbon atoms and heteroatoms of the heterocycloalkyl group can be optionally substituted by oxo or sulfido.

As used herein, the term “heterocycloalkylalkyl” refers to a C₁₋₆alkyl substituted by heterocycloalkyl.

As used herein, the term “hydroxy” or “hydroxyl” means an —OH group.

As used herein, the term “hydroxyalkyl” or “hydroxylalkyl” means an alkyl group substituted by a hydroxyl group. Examples of a hydroxylalkyl include, but are not limited to, —CH₂OH and —CH₂CH₂OH.

As used herein, the term “individual” or “patient,” used interchangeably, means any animal, including mammals, such as mice, rats, other rodents, rabbits, dogs, cats, swine, cattle, sheep, horses, or primates, such as humans.

As used herein, the phrase “inhibiting activity,” such as enzymatic or receptor activity means reducing by any measurable amount the activity of an enzyme or receptor, such as the S1P1 receptor.

As used herein, the phrase “activating activity,” such as enzymatic or receptor activity means increasing by any measurable amount the activity of an enzyme or receptor, such as the S1P1 receptor.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent. In some embodiments, the subject or patient that is “in need thereof” is a subject that has been diagnosed with, or is suspected of having, epilepsy or an epilepsy syndrome. In some embodiments, the subject or patient that is “in need thereof” has had a seizure. In some embodiments, the subject or patient that is “in need thereof” is currently having a seizure when the compounds or compositions provided herein are administered or used in the presently described methods.

As used herein, the phrase “in situ gellable” means embracing not only liquids of low viscosity that form gels upon contact with the eye or with lacrimal fluid in the exterior of the eye, but also more viscous liquids such as semi-fluid and thixotropic gels that exhibit substantially increased viscosity or gel stiffness upon administration to the eye.

As used herein, the phrase “integer from X to Y” means any integer that includes the endpoints. For example, the phrase “integer from X to Y” means 1, 2, 3, 4, or 5.

As used herein, the phrase “intractable epilepsy” refers to epilepsies or seizures associated therewith corresponding to the following four epilepsies or seizures associated therewith:

(1) epilepsies difficult to treat in which suppression of seizures associated therewith cannot be controlled through a conventional pharmaceutical treatment (Masako WATANABE, et al., Igaku-no Ayumi, 183(1):103-108, 1997); (2) epilepsies corresponding to the following (a) to (c): (a) localization-related epilepsies such as temporal lobe epilepsies and cortical epilepsies; (b) generalized epilepsies and myoclonic epilepsy; and (c) epilepsies and syndromes undetermined, whether focal or generalized, such as severe myoclonic epilepsy; (3) seizures associated with the above-described intractable epilepsies including tonic seizures, tonic-clonic seizures, atypical absence seizures, atonic seizures, myoclonic seizures, clonic seizures, simple partial seizures, complex partial seizures, and secondary generalized seizures; and (4) epilepsies such as epilepsies following brain surgery, traumatic epilepsies, and relapsed epilepsies following surgery for epilepsy. The characteristics of intractable epilepsy include high occurrence of partial seizure followed by a generalized seizure (particularly temporal lobe epilepsy), high occurrence of symptomatic epilepsy caused by an organic lesion in the brain, and long-term absence of treatment from the onset to consultation of a specialist and high occurrence of seizures; and high occurrence of status epilepticus in the anamnesis. The temporal lobe is likely to be a portion of the brain responsible for intractable epilepsy. It is indicated that epilepsy becomes more intractable by changing of the nature thereof and evolving as acquired seizures are repeated. Intractable epilepsy is categorized into three clinical types: (a) localization-related epilepsies and syndromes including temporal lobe epilepsies, frontal lobe epilepsies, and multi-lobe epilepsies wherein temporal lobe epilepsies and frontal lobe epilepsies are typical examples of intractable epilepsy and multi-lobe epilepsies are considered to be caused by two or more lobes; (b) generalized epilepsies and syndromes including myoclonic epilepsy; and (c) epilepsies and syndromes undetermined, whether focal or generalized, including severe myoclonic epilepsy, which exhibits a variety of seizure types including tonic-clonic seizures that frequently occur and often lead to status. Special treatment conducted by a specialist for epilepsy is strongly required (Masako WATANABE, et al., Igakuno Ayumi, 183(1):103-108, 1997). As used herein, the term “isolated” means that the compounds described herein are separated from other components of either (a) a natural source, such as a plant or cell, or (b) a synthetic organic chemical reaction mixture, such as by conventional techniques.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “N-alkyl” refers to a alkyl chain that is substituted with an amine group. Non-limiting examples, include, but are not limited to

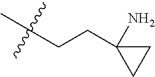 [SMILES: *CCC1(N)CC1]

and the like. The alkyl chain can be linear, branched, cyclic, or any combination thereof. In some embodiments, the alkyl comprises 1-10, 1-9, 1-8, 1-7, 1-6, 1-5, 1-4, 1-3, or 1-2 carbons.

As used herein, the term “nitro” means —NO₂.

As used herein, the term “n-membered”, where n is an integer, typically describes the number of ring-forming atoms in a moiety, where the number of ring-forming atoms is n. For example, pyridine is an example of a 6-membered heteroaryl ring and thiophene is an example of a 5-membered heteroaryl ring.

As used herein, the phrase “ophthalmically acceptable” means having no persistent detrimental effect on the treated eye or the functioning thereof, or on the general health of the subject being treated. However, it will be recognized that transient effects such as minor irritation or a “stinging” sensation are common with topical ophthalmic administration of drugs and the existence of such transient effects is not inconsistent with the composition, formulation, or ingredient (e.g., excipient) in question being “ophthalmically acceptable” as herein defined.

As used herein, the phrase “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent groups, provided that the normal valency of the designated atom or moiety is not exceeded, and that the substitution results in a stable compound. For example, if a methyl group is optionally substituted, then 3 hydrogen atoms on the carbon atom can be replaced with substituent groups.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

In some embodiments, the salt of a compound described herein is a pharmaceutically acceptable salt thereof. As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. The present embodiments also includes quaternary ammonium salts of the compounds described herein, where the compounds have one or more tertiary amine moiety.

As used herein, the term “phenyl” means —C₆H₅. A phenyl group can be unsubstituted or substituted with one, two, or three suitable substituents.

As used herein, the term “prodrug” means a derivative of a known direct acting drug, which derivative has enhanced delivery characteristics and therapeutic value as compared to the drug, and is transformed into the active drug by an enzymatic or chemical process.

As used herein, the term “purified” means that when isolated, the isolate contains at least 90%, at least 95%, at least 98%, or at least 99% of a compound described herein by weight of the isolate.

As used herein, the phrase “quaternary ammonium salts” means derivatives of the disclosed compounds with one or more tertiary amine moieties wherein at least one of the tertiary amine moieties in the parent compound is modified by converting the tertiary amine moiety to a quaternary ammonium cation via alkylation (and the cations are balanced by anions such as Cl⁻, CH₃COO⁻, and CF₃COO⁻), for example methylation or ethylation.

As used herein, the term “semicarbazone” means ═NNHC(═O)NH₂.

As used herein, the phrase “solubilizing agent” means agents that result in formation of a micellar solution or a true solution of the drug.

As used herein, the term “secondary generalized seizure” means one of the symptoms associated with intractable epilepsy, is one type of partial seizure, which exhibit a clinical syndrome and an electrocephalogram feature observed as excitation of neurons that shows initiation of a seizure in a limited portion of one cerebral hemisphere. The secondary generalized seizure is initiated as a simple partial seizure (without impairment of consciousness) or a complex partial seizure (with impairment of consciousness), and develops to general convulsion induced through secondary generalization. The main symptom thereof is convulsion such as a tonic-clonic seizure, a tonic seizure, or a clonic seizure.

As used herein, the term “solution/suspension” means a liquid composition wherein a first portion of the active agent is present in solution and a second portion of the active agent is present in particulate form, in suspension in a liquid matrix.

As used herein, the term “temporal lobe epilepsy,” which is one type of intractable epilepsy, is an epilepsy having a seizure focus in the temporal lobe, and is categorized under symptomatic and localization-related epilepsies, which also include frontal lobe epilepsies, parietal lobe epilepsies, and occipital lobe epilepsies, based on the international classification of epilepsy. The syndromes of temporal lobe epilepsy vary in accordance with a focus-localized site and type of seizure propagation, in that the temporal lobe has an anatomically complex structure including neocortex, allocortex, and paleocortex. Temporal lobe epilepsy, as previously defined as a psychomotor seizure, mostly causes complex partial seizures as clinically observed seizures, and also causes simple partial seizures, secondary generalized seizures, and combinations thereof. Simple partial seizures include autonomic and mental symptoms and sensory symptoms such as olfaction, audition, or vision, sometimes concomitant with symptoms of experiences such as deja-vu and jamais-vu. Complex partial seizures often exhibit motion stopping followed by eating-function automatism, and are divided into amygdala-hippocampus seizures and lateral temporal lobe seizures according to localization. In the case of temporal lobe epilepsy, 70-80% of the seizures are hippocampus seizures, in which aura, motion stopping, lip automatism, and clouding of consciousness are successively developed to result in amnesia. When the focus is in the amygdala, there are caused autonomic symptoms such as dysphoria in the epigastrium; phobia; and olfactory hallucination. Lateral temporal lobe seizures include auditory illusion, hallucination, and a dreamy state, and disturbance of speech when the focus is in the dominant hemisphere. Temporal lobe epilepsy exhibits a long-term psychosis-like state in addition to other symptoms and recognition-and-memory disorder more frequently than do other epilepsies (Medical Dictionary, Nanzando). Treatment of temporal lobe epilepsy is carried out through pharmacotherapy employing a maximum dose of a combination of drugs, or through surgical treatment.

As used herein, the phrase “substantially isolated” means a compound that is at least partially or substantially separated from the environment in which it is formed or detected.

As used herein, the phrase “suitable substituent” or “substituent” means a group that does not nullify the synthetic or pharmaceutical utility of the compounds described herein or the intermediates useful for preparing them. Examples of suitable substituents include, but are not limited to: C₁-C₆alkyl, C₁-C₆alkenyl, C₁-C₆alkynyl, C₅-C₆aryl, C₁-C₆alkoxy, C₃-C₅heteroaryl, C₃-C₆cycloalkyl, C₅-C₆aryloxy, —CN, —OH, oxo, halo, haloalkyl, —NO₂, —CO₂H, —NH₂, —NH(C₁-C₈alkyl), —N(C₁-C₈alkyl)₂, —NH(C₆aryl), —N(C₅-C₆aryl)₂, —CHO, —CO(C₁-C₆alkyl), —CO((C₅-C₆)aryl), —CO₂((C₁-C₆)alkyl), and —CO₂((C₅-C₆)aryl). One of skill in art can readily choose a suitable substituent based on the stability and pharmacological and synthetic activity of the compounds described herein.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

As used herein, the term “traumatic epilepsy,” which is one type of intractable epilepsy, in a broad sense, is divided into two epilepsies, i.e., “early epilepsy” and “late epilepsy.” “Early epilepsy” is caused through stimulation of the brain induced by convulsion within a week after suffering a trauma, and is not a true epilepsy. In contrast, “late epilepsy” is a true epilepsy that is caused one or more weeks after suffering a trauma. Most of the traumatic epilepsies are caused by formation of a focus at a traumatically damaged portion of the cortex, and they are considered to be typical examples of partial epilepsies.

As used herein, the terms “treat,” “treated,” or “treating” mean both therapeutic treatment and prophylactic measures wherein the object is to slow down (lessen) an undesired pathophysiological condition, disorder or disease, or obtain beneficial or desired clinical results. Beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment. Thus, “treatment of epilepsy” or “treating epilepsy” means an activity that alleviates or ameliorates any of the primary phenomena or secondary symptoms associated with the epilepsy or other condition described herein.

As used herein, the term “ureido” means —NHC(═O)—NH₂.

At various places in the present specification, substituents of compounds may be disclosed in groups or in ranges. It is specifically intended that embodiments include each and every individual subcombination of the members of such groups and ranges. For example, the term “C₁₋₆alkyl” is specifically intended to individually disclose methyl, ethyl, propyl, C₄alkyl, C₅alkyl, and C₆alkyl.

For compounds in which a variable appears more than once, each variable can be a different moiety selected from the Markush group defining the variable. For example, where a structure is described having two R groups that are simultaneously present on the same compound, the two R groups can represent different moieties selected from the Markush groups defined for R. In another example, when an optionally multiple substituent is designated in the form, for example,

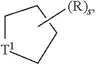

then it is understood that substituent R can occur s number of times on the ring, and R can be a different moiety at each occurrence. In the above example, where the variable T¹ is defined to include hydrogens, such as when T¹ is CH₂, NH, etc., any H can be replaced with a substituent.

It is further appreciated that certain features described herein, which are, for clarity, described in the context of separate embodiments, can also be provided in combination in a single embodiment. Conversely, various features, which are, for brevity, described in the context of a single embodiment, can also be provided separately or in any suitable subcombination.

It is understood that the present embodiments encompasses the use, where applicable, of stereoisomers, diastereomers and optical stereoisomers of the compounds, as well as mixtures thereof. Additionally, it is understood that stereoisomers, diastereomers, and optical stereoisomers of the compounds, and mixtures thereof, are within the scope of the embodiments. By way of non-limiting example, the mixture may be a racemate or the mixture may comprise unequal proportions of one particular stereoisomer over the other. Additionally, the compounds can be provided as a substantially pure stereoisomers, diastereomers and optical stereoisomers (such as epimers).

The compounds described herein can be asymmetric (e.g., having one or more stereocenters). All stereoisomers, such as enantiomers and diastereomers, are intended to be included within the scope of the embodiments unless otherwise indicated. Compounds that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are provided herein. Cis and trans geometric isomers of the compounds are also included within the present embodiments and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

In some embodiments, the composition comprises a compound, or a pharmaceutically acceptable salt thereof, that is at least 90%, at least 95%, at least 98%, or at least 99%, or 100% enantiomeric pure, which means that the ratio of one enantiomer to the other in the composition is at least 90:1 at least 95:1, at least 98:1, or at least 99:1, or is completely in the form of one enantiomer over the other.

Resolution of racemic mixtures of compounds can be carried out by any of numerous methods known in the art, including, for example, chiral HPLC, fractional recrystallization using a chiral resolving acid which is an optically active, salt-forming organic acid. Suitable resolving agents for fractional recrystallization methods include, but are not limited to, optically active acids, such as the D and L forms of tartaric acid, diacetyltartaric acid, dibenzoyltartaric acid, mandelic acid, malic acid, lactic acid, and the various optically active camphorsulfonic acids such as β-camphorsulfonic acid. Other resolving agents suitable for fractional crystallization methods include, but are not limited to, stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

Compounds may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds also include hydrates and solvates, as well as anhydrous and non-solvated forms.

Compounds can also include all isotopes of atoms occurring in the intermediates or final compounds. Isotopes include those atoms having the same atomic number but different mass numbers. For example, isotopes of hydrogen include tritium and deuterium.

In some embodiments, the compounds, or salts thereof, are substantially isolated. Partial separation can include, for example, a composition enriched in the compound. Substantial separation can include compositions containing at least about 50%, at least about 60%, at least about 70%, at least about 80%, at least about 90%, at least about 95%, at least about 97%, or at least about 99% by weight of the compound, or salt thereof. Methods for isolating compounds and their salts are routine in the art.

Although the disclosed compounds are suitable, other functional groups can be incorporated into the compound with an expectation of similar results. In particular, thioamides and thioesters are anticipated to have very similar properties. The distance between aromatic rings can impact the geometrical pattern of the compound and this distance can be altered by incorporating aliphatic chains of varying length, which can be optionally substituted or can comprise an amino acid, a dicarboxylic acid or a diamine. The distance between and the relative orientation of monomers within the compounds can also be altered by replacing the amide bond with a surrogate having additional atoms. Thus, replacing a carbonyl group with a dicarbonyl alters the distance between the monomers and the propensity of dicarbonyl unit to adopt an anti-arrangement of the two carbonyl moiety and alter the periodicity of the compound. Pyromellitic anhydride represents still another alternative to simple amide linkages, which can alter the conformation and physical properties of the compound. Modern methods of solid phase organic chemistry (E. Atherton and R. C. Sheppard, Solid Phase Peptide Synthesis A Practical Approach IRL Press Oxford 1989) now allow the synthesis of homodisperse compounds with molecular weights approaching 5,000 Daltons. Other substitution patterns are equally effective.

The compounds also include derivatives referred to as prodrugs.

Compounds containing an amine function can also form N-oxides. A reference herein to a compound that contains an amine function also includes the N-oxide. Where a compound contains several amine functions, one or more than one nitrogen atom can be oxidized to form an N-oxide. Examples of N-oxides include N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g., a peroxycarboxylic acid) (see, Advanced Organic Chemistry, by Jerry March, 4th Edition, Wiley Interscience).

Embodiments of various compounds and salts thereof for methods of treating or preventing a seizure, an epilepsy or an epilepsy-related syndrome in a subject as described herein are provided. Where a variable is not specifically recited, the variable can be any option described herein, except as otherwise noted or dictated by context.

In some embodiments, the compound is as described in the appended exemplary, non-limiting claims, or a pharmaceutically acceptable salt thereof.

In some embodiments, compounds having Formula I or Formula II, or a pharmaceutically acceptable salt thereof, are provided:

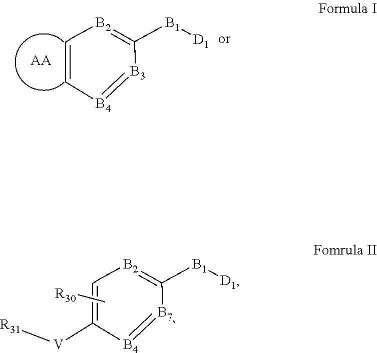

wherein:

AA is

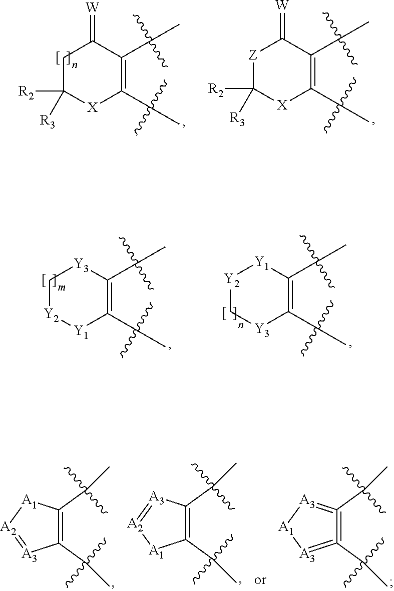

W is O, S, or NR1;

X is O, S, or NR₄;

V is O, S, or NR₃₂;

Z is CHR₄₂ or NR₄₃;

n is 0, 1, 2, 3, or 4;

Y₁ and Y₂ are independently O, S, NR₅, C═O, C═S or C═NR₆;

Y₃ is O, S, CH₂, or NR₃₄;

m is 0, 1, 2, or 3;

A₁ is O, S, NR₇, C═O, or C═S;

A₂ and A₃ are independently CR₂₉ or N;

B₁ is an optionally substituted aryl or heteroaryl group, a carbocycle, or

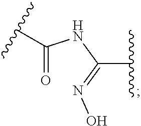 [SMILES: *C(=O)N/C(*)=N/O]

B₂, B₃, and B₄ are independently CR₃₈ or N;

D₁ is H, OH, NH₂, NO₂, cycle, optionally substituted aryl group, branched or unbranched alkyl alcohol, halo, branched or unbranched alkyl, amide, cyano, alkoxy, haloalkyl, aklylsulfonyl, nitrite, or alkylsulfanyl;

R₂ and R₃, are independently H, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₂ and R₃ are together optionally substituted cycloalkyl, or optionally substituted cycloheteroalkyl;

R₁, R₄, R₅, R₆, R₇, R₂₉, R₃₁, R₃₂, R₃₃, R₃₄, R₃₈, and R₄₃ are independently H, OH, NH₂, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl.

R₃₀ is independently H, CN, CF₃, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or optionally substituted haloalkyl;

R₄₂ is independently Br, Cl, F, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl;

In some embodiments of compounds of Formula I or Formula II, D₁ and B₁ are:

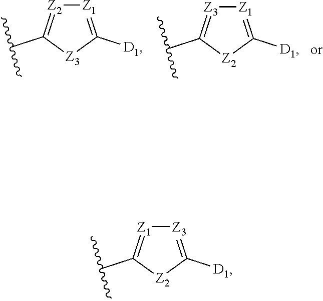

wherein:

Z₁ and Z₂ are independently N or CR₃₉;

Z₃ is O, S, or NR₂₇;

R₂₇ and R₃₉ are independently H, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl, and

D₁ is H, OH, NH₂, NO₂, cycle, optionally substituted aryl group, branched or unbranched alkyl alcohol, halo, branched or unbranched alkyl, amide, cyano, alkoxy, haloalkyl, aklylsulfonyl, nitrite, or alkylsulfanyl.

In some embodiments, one of Z₁ and Z₂ is N. In some embodiments, both Z₁ and Z₂ are N. In some embodiments, Z₃ is O.

In some embodiments of compounds, or a pharmaceutically acceptable salt thereof, of Formula I or Formula II, D₁ and B₁ have a formula of

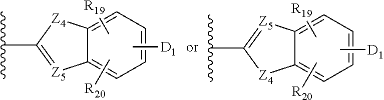

wherein:

Z₄ is O, S, or NR₂₈;

Z₅ is N or CH;

R₁₉, and R₂₀ are each independently H, OH, NH₂, NO₂, cycle, aryl, branched or unbranched alkyl alcohol, halo, branched or unbranched alkyl, amide, cyano, alkoxy, alkylthio, haloalkyl, aklylsulfonyl, nitrite, or alkylsulfanyl; or two of R₁₉, and R₂₀ together form an aryl or cycle that is attached to one or more of the atoms of B₁;

R₂₈ is H, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl, and

D₁ is H, OH, NH₂, NO₂, cycle, optionally substituted aryl group, branched or unbranched alkyl alcohol, halo, branched or unbranched alkyl, amide, cyano, alkoxy, haloalkyl, aklylsulfonyl, nitrite, or alkylsulfanyl.

In some embodiments, Z₅ is N. In some embodiments, Z₄ is O. In some embodiments, Z₅ is N and Z₄ is O.

In some embodiments of compounds of Formula I or Formula II, D₁ is

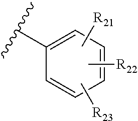

wherein R₂₁, R₂₂, and R₂₃ are each independently H, OH, NH₂, NO₂, cycle, aryl, branched or unbranched alkyl alcohol, halo, branched or unbranched alkyl, amide, cyano, alkoxy, haloalkyl, aklylsulfonyl, nitrite, or alkylsulfanyl; or two of R₂₁, R₂₂, and R₂₃ together form an aryl or cycle that is attached to one or more of the atoms of D₁.

In some embodiments, one of R₂₁, R₂₂, and R₂₃ is H. In some embodiments, two of R₂₁, R₂₂, and R₂₃ are H. In some embodiments, R₂₃ is Me, OH, NH₂, Cl, NHSO₂Me, SO₂NH₂, NH(CO)Me, or (CO)NH₂. In some embodiments, R₂₁ and R₂₂ are H and R₂₃ is Me, OH, NH₂, Cl, NHSO₂Me, SO₂NH₂, NH(CO)Me, or (CO)NH₂.

In some embodiments of compounds of Formula I or Formula II, D₁ is optionally substituted aryl or optionally substituted hetero aryl.

In some embodiments of compounds of Formula I or Formula II, D₁ is

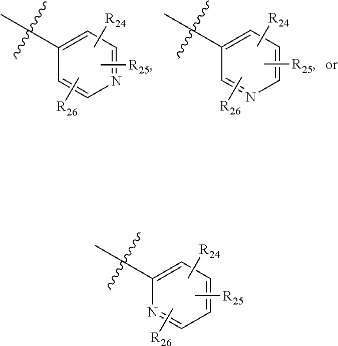

wherein R₂₄, R₂₅, and R₂₆ are each independently H, OH, NH₂, NO₂, cycle (e.g. carbocycle or heterocycle), aryl, branched or unbranched alkyl alcohol, halo, branched or unbranched alkyl, amide, cyano, alkoxy, haloalkyl, aklylsulfonyl, nitrite, or alkylsulfanyl; or two of R₂₄, R₂₅, and R₂₆ together form an aryl or cycle that is attached to one or more of the atoms of D₁.

In some embodiments, one of R₂₄, R₂₅, and R₂₆ is H. In some embodiments, two of R₂₄, R₂₅, and R₂₆ are H. In some embodiments, R₂₆ is H, Me, OH, CF₃, or OMe. In some embodiments, R₂₄ and R₂₅ are H and R₂₆ is H, Me, OH, CF₃, or OMe.

In some embodiments of compounds of Formula I or Formula II, AA is

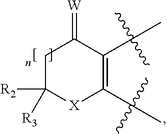

wherein the variables are as defined in the preceding embodiments.

In some embodiments, W is O. In some embodiments, X is O. In some embodiments, R₂ and R₃ are independently H, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, R₂ and R₃ are the same. In some embodiments, R₂ and R₃ are ethyl.

In some embodiments, D₁ is

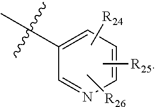

In some embodiments, one of R₂₄, R₂₅, and R₂₆ is H. In some embodiments, two of R₂₄, R₂₅, and R₂₆ are H and the other member is as defined herein. In some embodiments, D₁ is

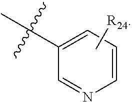

In some embodiments, D₁ is

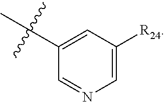

In some embodiments, R₂₄ is H. In some embodiments, R₂₄ is OH. In some embodiments, D₁ is

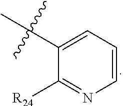

In some embodiments, R₂₄ is OMe.

In some embodiments, D₁ is

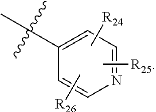

In some embodiments, one of R₂₄, R₂₅, and R₂₆ is H. In some embodiments, two of R₂₄, R₂₅, and R₂₆ are H and the other member is as defined herein.

In some embodiments, D₁ is

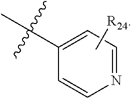

In some embodiments, D₁ is

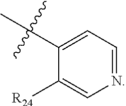

In some embodiments, D₁ is

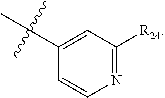

In some embodiments, R₂₄ is halide. In some embodiments, R₂₄ is F.

In some embodiments, R₂₄ is Me. In some embodiments, R₂₄ is OMe. In some embodiments, R₂₄ is OH.

In some embodiments of compounds of Formula I or Formula II, R₂ and R₃ are together

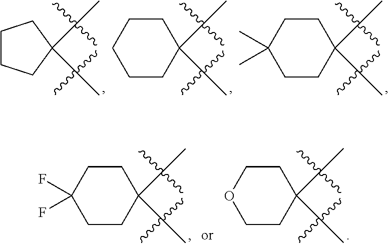

In some embodiments, n is 1.

In some embodiments of compounds of Formula I or Formula II, AA is

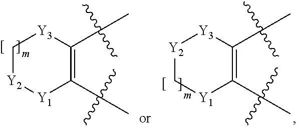

wherein the variables are as defined in the preceding embodiments.

In some embodiments, Y₁ is NR₅. In some embodiments, R₅ is H.

In some embodiments, Y₂ is C═NR₆. In some embodiments, R₆ is H.

In some embodiments, Y₂ is C═O. In some embodiments, Y₃ is O. In some embodiments, Y₃ is CH₂. In some embodiments, m is 0. In some embodiments, m is 1.

In some embodiments of compounds of Formula I or Formula II, AA is

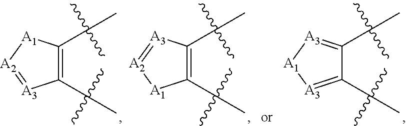

wherein the variables are as defined in the preceding embodiments.

In some embodiments, A₁ is O. In some embodiments, A₁ is S. In some embodiments, A₂ is N. In some embodiments, A₃ is N. In some embodiments, A₃ is CR₂₉. In some embodiments, R₂₉ is H.

In some embodiments, A₂ is CR₂₉. In some embodiments, R₂₉ is H.

In some embodiments, A₁ is NR₇. In some embodiments, R₇ is

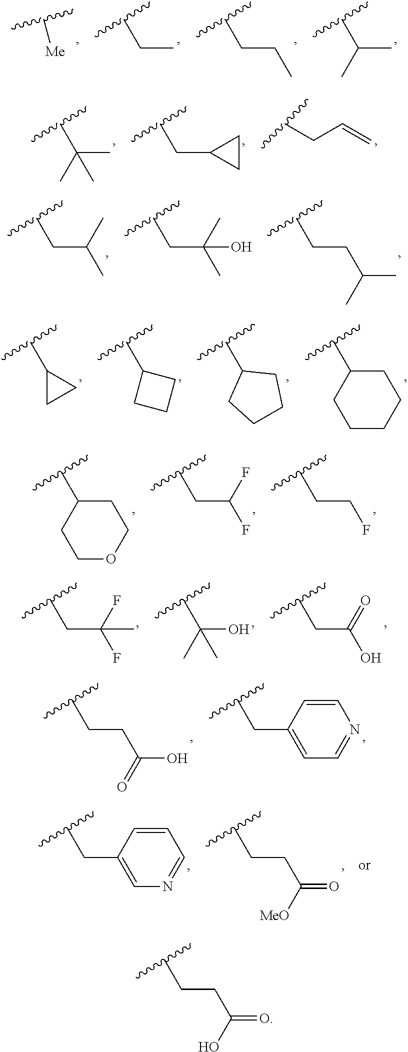

In some embodiments, D₁ is

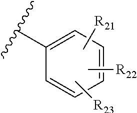

and one of R₂₁, R₂₂, and R₂₃ is H.

In some embodiments, D₁ is

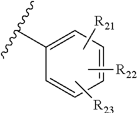

and two of R₂₁, R₂₂, and R₂₃ are H. In some embodiments, D₁ is

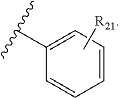

In some embodiments, R₂₁ is optionally substituted C₁-C₆ alkyl. In some embodiments, R₂₁ is ethyl or methyl. In some embodiments, D₁ is

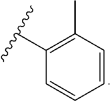 [SMILES: *c1ccccc1C]

In some embodiments of compounds of Formula I or Formula II, D₁ is

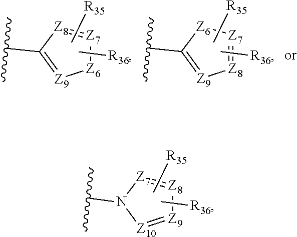

wherein:

Z₆ is O, S, NR₄₀, or CHR₃₇;

Z₇, Z₈, Z₉ and Z₁₀ are independently N or CR₄₁;

R₃₅, R₃₆, R₃₇, R₄₀, and R₄₁ are each independently H, OH, NH₂, cycle, aryl, branched or unbranched alkyl alcohol, halo, branched or unbranched alkyl, amide, cyano, alkoxy, haloalkyl, aklylsulfonyl, nitrite, or alkylsulfanyl; or R₃₅ and R₃₆ together form an aryl or cycle that is attached to one or more of the atoms of D₁.

In some embodiments, one of R₃₅ and R₃₆ is H.

In some embodiments, both R₃₅ and R₃₆ are H. In some embodiments, Z₆ is NH. In some embodiments, one of Z₇, Z₈ and Z₉ is N.

In some embodiments, Z₇ is N. In some embodiments, Z₈ is CH. In some embodiments, Z₉ is CH. In some embodiments, both Z₈ and Z₉ are CH.

In some embodiments, AA is

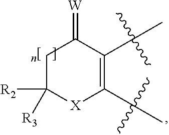

wherein the variables are as defined in the preceding embodiments.

In some embodiments, W is O. In some embodiments, X is O. In some embodiments, R₂ and R₃ are independently H, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, both R₂ and R₃ are the same. In some embodiments, both R₂ and R₃ are methyl or ethyl. In some embodiments, n is 1. In some embodiments, D₁ is pyrazolyl. In some embodiments, D₁ is

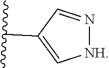 [SMILES: *c1cn[nH]c1]

In some embodiments, the compound, or a pharmaceutically acceptable salt thereof, is a compound of Formula I having a formula of

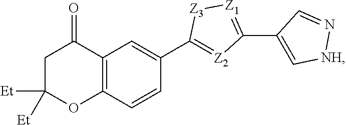

or a pharmaceutically acceptable salt thereof, wherein Z₁, Z₂, and Z₃ are as defined herein and above.

In some embodiments, Z₂ is N. In some embodiments, Z₁ is N. In some embodiments, Z₃ is O. In some embodiments, Z₂ and Z₁ are N and Z₃ is as defined herein. In some embodiments, Z₂ and Z₁ are N and Z₃ is O. In some embodiments, the compound is a compound of Formula I having a formula of

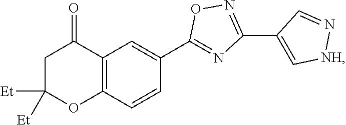 [SMILES: CCC1(CC)CC(=O)c2cc(-c3nc(-c4cn[nH]c4)no3)ccc2O1]

or a pharmaceutically acceptable salt thereof.

In some embodiments of compounds of Formula II, D₁ and B₁ is

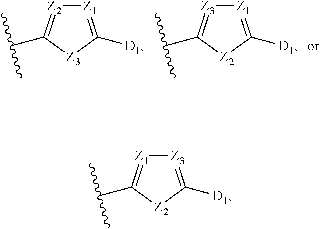

and the variables are as defined in the preceding embodiments.

In some embodiments, Z₃ is O and Z₁ and Z₂ are independently N or CR₃₉.

In some embodiments, Z₁ is N, Z₂ is N or CR₃₉ and Z₃ is O, S, or NR₂₇. In some embodiments, Z₁ and Z₂ are N and Z₃ is O.

In some embodiments, the compound is a compounds of Formula II having a formula of

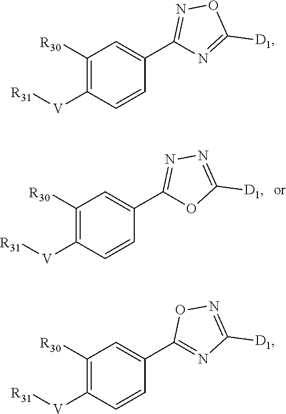

or a pharmaceutically acceptable salt thereof, wherein the variables are as defined in the preceding embodiments. In some embodiments, R₃₀ is CN. In some embodiments, V is NH. In some embodiments, R₃₁ is C₁-C₅ alkyl. In some embodiments, R₃₁ is

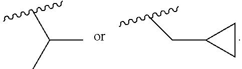

In some embodiments, R₃₁ is C₁-C₅ haloalkyl.

In some embodiments, R₃₁ is

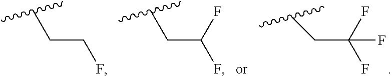

In some embodiments of compounds of Formula II, D₁, B₁, and AA together is

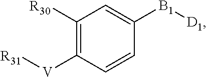

wherein the variables are as defined in the preceding embodiments.

In some embodiments, R₃₀ is CF₃. In some embodiments, V is O or NH.

In some embodiments, R₃₀ is CF₃.

In some embodiments, B₁-D₁ is

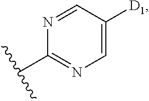

wherein D1 is as defined herein and above. In some embodiments, D₁ is

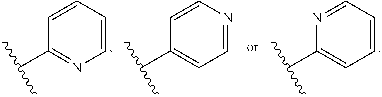

In some embodiments, R₃₁ is

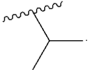 [SMILES: *C(C)C]

In the preceding embodiments, or as shown below, or as illustrated in the appending claims, if a variable (substituent) is not explicitly defined then the variable is as defined above, which would be readily apparent based upon the present embodiments.

In some embodiments, the compound has a formula of:

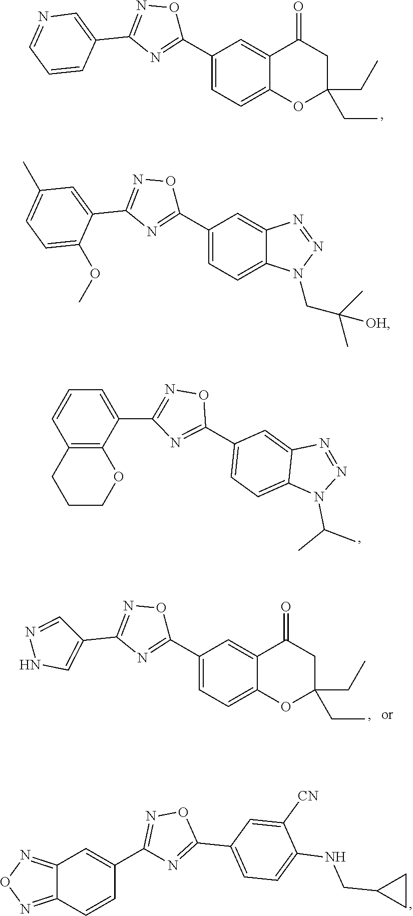

or N, or a pharmaceutically acceptable salt thereof.

In some embodiments, the present embodiments provide methods of treating or preventing a seizure, an epilepsy or an epilepsy-related syndrome, and the like as described herein in a subject, methods comprising administering to the subject a pharmaceutical composition comprising one or more compounds as provided or described herein, such as any compound of Formula I or Formula II, or a pharmaceutically acceptable salt thereof.

In some embodiments, the present embodiments provide methods of treating or preventing a seizure, an epilepsy or an epilepsy-related syndrome, and the like as described herein in a subject, methods comprising administering to the subject a pharmaceutical composition comprising one or more compounds as provided or described herein and a pharmaceutically acceptable carrier.

In some embodiments, the present embodiments provide methods of treating or preventing a seizure, an epilepsy or an epilepsy-related syndrome, and the like as described herein in a subject, methods comprising administering to the subject one or more compounds described herein, or a pharmaceutically acceptable salt thereof, or a pharmaceutical composition comprising one or more compounds described herein. In some embodiments, the present embodiments provide methods of treating a seizure, an epilepsy or an epilepsy-related syndrome, and the like as described herein in a subject, methods comprising administering to the subject one or more compounds described herein, or a pharmaceutically acceptable salt thereof, or a pharmaceutical composition comprising one or more compounds described herein. In some embodiments, the present embodiments provide methods of preventing a seizure, or a symptom related to epilepsy or an epilepsy-related syndrome, and the like as described herein in a subject, methods comprising administering to the subject one or more compounds described herein, or a pharmaceutically acceptable salt thereof, or a pharmaceutical composition comprising one or more compounds described herein.

is an intractable epilepsy.

In some embodiments, the epilepsy that is being treated is intractable epilepsy. In some embodiments, the intractable epilepsy is localization-related epilepsy, generalized epilepsy or syndromes thereof. In some embodiments, the localization-related epilepsy is cortical epilepsy or temporal lobe epilepsy. In some embodiments, the cortical epilepsy is frontal lobe epilepsy, parietal lobe epilepsy, or occipital lobe epilepsy. In some embodiments, the methods are used to treat or prevent an epileptic seizure. In some embodiments, the epileptic seizure is an intractable localization-related epilepsy seizure, an intractable secondary generalized seizure, an intractable complex partial seizure, or an intractable status epilepticus.

In some embodiments, the epilepsy is an intractable epilepsy. In some embodiments, wherein the intractable epilepsy is localization-related epilepsy, generalized epilepsy or syndromes thereof. In some embodiments, the localization-related epilepsy is cortical epilepsy or temporal lobe epilepsy. In some embodiments, the cortical epilepsy is frontal lobe epilepsy, parietal lobe epilepsy, or occipital lobe epilepsy. In some embodiments, wherein the epilepsy-related syndrome is an epileptic seizure. In some embodiments, the epileptic seizure is an intractable localization-related epilepsy, an intractable secondary generalized seizure, an intractable complex partial seizure or an intractable status epilepticus.

In some embodiments, the present embodiments provide methods of treating or preventing an epilepsy or an epilepsy-related syndrome in a subject, the method further comprises at least one (i.e, additional) anti-epilepsy drug that is not a compound of Formula I or Formula II. In some embodiments, the at least one anti-epilepsy drug is selected from the group consisting of carbamazepine, clonazepam, eslicarbazepine, felbamate, gabapentin, lacosamide, lamotrigine, levetiracetam, oxcarbazepine, phenobarbital, phenytoin, pregabalin, primidone, rufinamide, tiagabine, topiramate, vigabatrin, valproic acid, and zonisamide.

In some embodiments, wherein the subject is a subject in need thereof. In some embodiments, wherein the epilepsy therapeutic is selected from those described herein.

In some embodiments, the condition is prevented.

In some embodiments, a compound, or a pharmaceutically acceptable salt thereof, for methods of treating or preventing a seizure, an epilepsy or an epilepsy-related syndrome, and the like as described herein is chosen from a compound of as shown in the following table and/or as described herein, including in the Examples section of the present disclosure. Any of the compounds provided for herein can be prepared as pharmaceutically acceptable salts and/or as part of a pharmaceutical composition as provided for herein. Examples of such salts are provided for herein. As described herein, the compounds can be prepared according to the schemes and methods described herein.

| | Compound | Chemical |
|---|---|---|
| Structure | Number | Name |
| 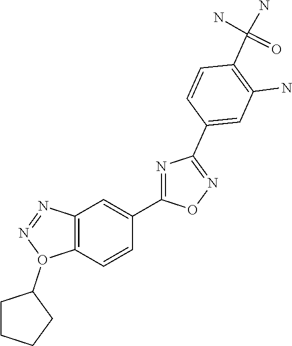 | 1 | 1-cyclopentyl-5-{3-[3-fluoro-4- (trifluoromethyl)phenyl]-1,2,4- oxadiazol-5-yl}-1H-1,2,3- benzotriazole |
| 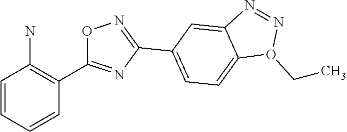 | 2 | 5-(2-bromophenyl)-3-(1-ethyl- 1H-benzo[d][1,2,3]triazol-5-yl)- 1,2,4-oxadiazole |
| 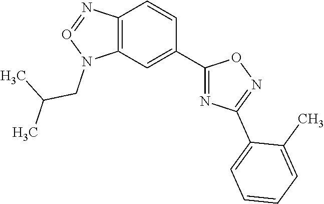 | 3 | 1-(2-methylpropyl)-6-[3-(4- methylpyridin-3-yl)-1,2,4- oxadiazol-5-yl]-1H-1,2,3- benzotriazole |
| 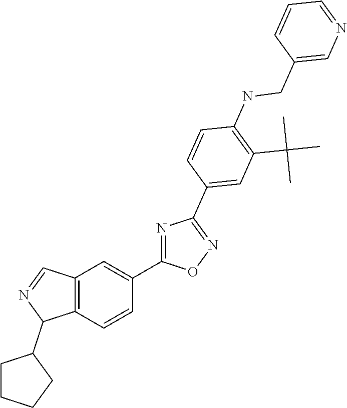 [SMILES: CC(C)(C)c1cc(-c2noc(-c3ccc4c(c3)C=NC4C3CCCC3)n2)ccc1NCc1cccnc1] | 4 | 1-cyclopentyl-5-{3-[4-(pyridin- ylmethoxy)-3- (trifluoromethyl)phenyl]-1,2,4 oxadiazol-5-yl}-1H-1,2,3- benzotriazole |
| 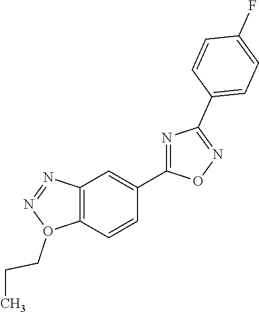 | 5 | 5-[3-(4-fluorophenyl)-1,2,4- oxadiazol-5-yl]-1-propyl-1H- 1,2,3-benzotriazole |
| 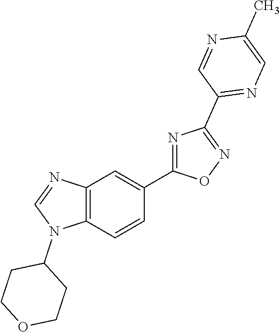 [SMILES: Cc1cnc(-c2noc(-c3ccc4c(c3)ncn4C3CCOCC3)n2)cn1] | 6 | 5-[3-(5-methylpyrazin-2-yl)- 1,2,4-oxadiazol-5-yl]-1-(oxan-4- yl)-1H-1,2,3-benzotriazole |
| 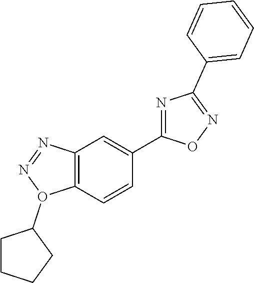 | 7 | 1-cyclopentyl-5-(3-phenyl-1,2,4- oxadiazol-5-yl)-1H-1,2,3- benzotriazole |
| 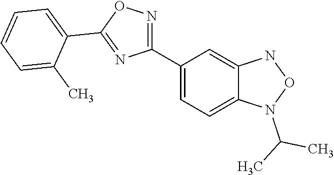 [SMILES: Cc1ccccc1-c1nc(-c2ccc3c(c2)NON3C(C)C)no1] | 8 | 3-(1-isopropyl-1H- benzo[d][1,2,3]triazol-5-yl)-5-(3- methylpyridin-4-yl)-1,2,4- oxadiazole |
| 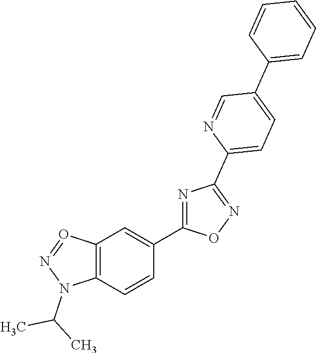 | 9 | 5-[3-(5-phenylpyridin-2-yl)-1,2,4- oxadiazol-5-yl]-1-(propan-2-yl)- 1H-1,2,3-benzotriazole |
| 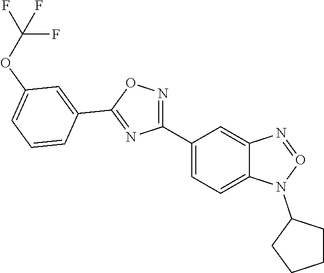 | 10 | 3-(1-cyclopentyl-1H- benzo[d][1,2,3]triazol-5-yl)-5-(3- (trifluoromethoxy)phenyl)-1,2,4- oxadiazole |
| 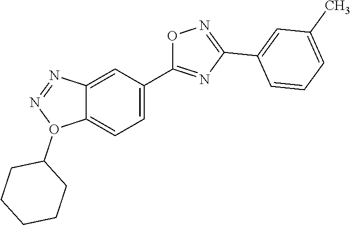 | 11 | 1-cyclohexyl-5-[3-(3- methylphenyl)-1,2,4-oxadiazol-5- yl]-1H-1,2,3-benzotriazole |
| 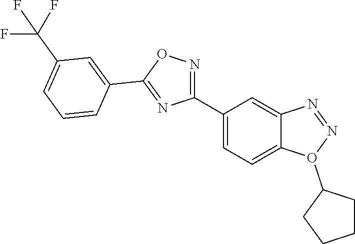 | 12 | 3-(1-cyclopentyl-1H- benzo[d][1,2,3]triazol-5-yl)-5-(3- (trifluoromethyl)phenyl)-1,2,4- oxadiazole |
| 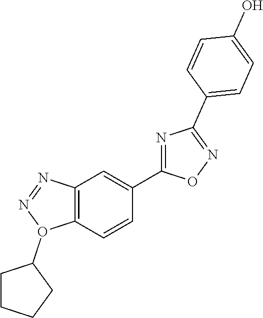 | 13 | 4-[5-(1-cyclopentyl-1H-1,2,3- benzotriazol-5-yl)-1,2,4- oxadiazol-3-yl]phenol |
| 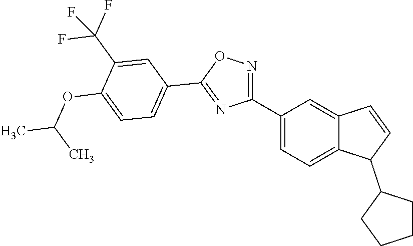 [SMILES: CC(C)Oc1ccc(-c2nc(-c3ccc4c(c3)C=CC4C3CCCC3)no2)cc1C(F)(F)F] | 14 | 3-(1-cyclopentyl-1H- benzo[d][1,2,3]triazol-5-yl)-5-(4- isopropoxy-3- (trifluoromethyl)phenyl)-1,2,4- oxadiazole |
| 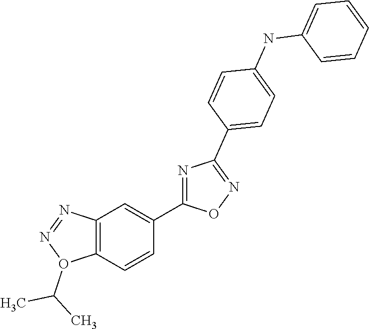 | 15 | 5-[3-(4-phenoxyphenyl)-1,2,4- oxadiazol-5-yl]-1-(propan-2-yl)- 1H-1,2,3-benzotriazole |
| 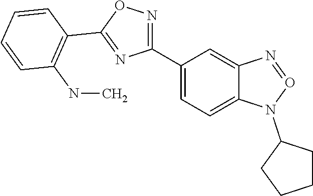 | 16 | 3-(1-cyclopentyl-1H- benzo[d][1,2,3]triazol-5-yl)-5-(2- (methylthio)phenyl)-1,2,4- oxadiazole |
| 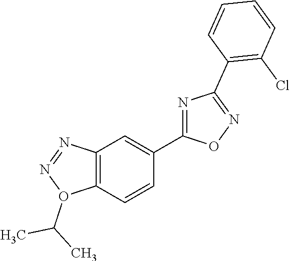 | 17 | 5-[3-(2-chlorophenyl)-1,2,4- oxadiazol-5-yl]-1-(propan-2-yl)- 1H-1,2,3-benzotriazole |
| 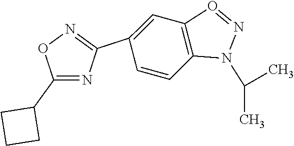 | 18 | 5-(5-cyclobutyl-1,2,4-oxadiazol- 3-yl)-1-(propan-2-yl)-1H-1,2,3- benzotriazole |
| 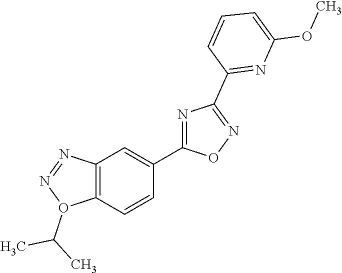 | 19 | 5-[3-(6-methoxypyridin-2-yl)- 1,2,4-oxadiazol-5-yl]-1-(propan- 2-yl)-1H-1,2,3-benzotriazole |
| 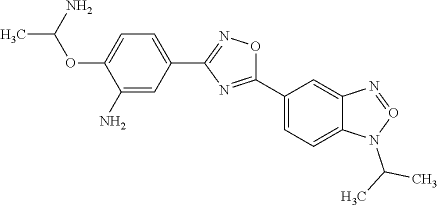 | 20 | 3-(4-isopropoxy-3- (trifluoromethyl)phenyl)-5-(1- isopropyl-1H- benzo[d][1,2,3]triazol-5-yl)-1,2,4- oxadiazole |
| 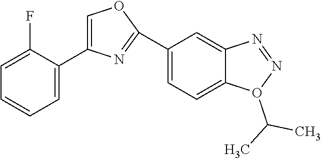 | 21 | 5-[4-(2-fluorophenyl)-1,3-oxazol- 2-yl]-1-(propan-2-yl)-1H-1,2,3- benzotriazole |
| 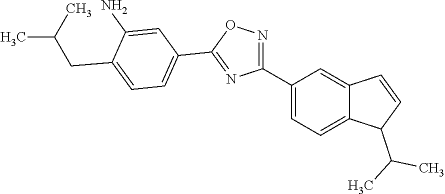 [SMILES: CC(C)Cc1ccc(-c2nc(-c3ccc4c(c3)C=CC4C(C)C)no2)cc1N] | 22 | 2-isopropoxy-5-(3-(1-isopropyl- 1H-benzo[d][1,2,3]triazol-5-yl)- 1,2,4-oxadiazol-5-yl)benzonitrile |
| 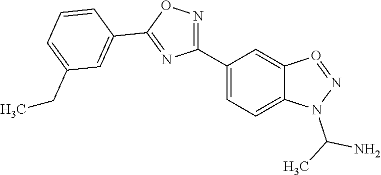 | 23 | 3-(1-isopropyl-1H- benzo[d][1,2,3]triazol-5-yl)-5-(3- (phenoxymethyl)phenyl)-1,2,4- oxadiazole |
| 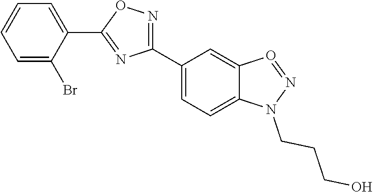 | 24 | 3-(5-(5-(2-bromophenyl)-1,2,4- oxadiazol-3-yl)-1H- benzo[d][1,2,3]triazol-1- yl)propan-1-ol |
| 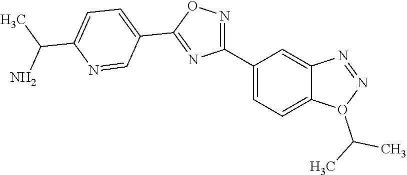 | 25 | 3-(1-isopropyl-1H- benzo[d][1,2,3]triazol-5-yl)-5-(6- isopropylpyridin-3-yl)-1,2,4- oxadiazole |
| 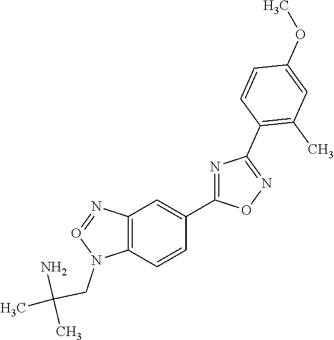 | 26 | 1-{5-[3-(4-methoxy-2- methylphenyl)-1,2,4-oxadiazol-5- yl]-1H-1,2,3-benzotriazol-1-yl}- 2-methylpropan-2-ol |
| 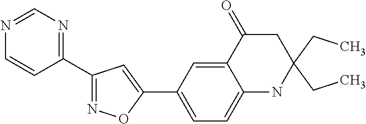 [SMILES: CCC1(CC)CC(=O)c2cc(-c3cc(-c4ccncn4)no3)ccc2N1] | 27 | 2,2-diethyl-6-[3-(pyridin-4-yl)- 1,2,4-oxadiazol-5-yl]-3,4- dihydro-2H-1-benzopyran-4-one |
| 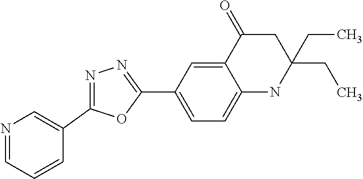 [SMILES: CCC1(CC)CC(=O)c2cc(-c3nnc(-c4cccnc4)o3)ccc2N1] | 28 | 2,2-diethyl-6-(5-(pyridin-3-yl)- 1,3,4-oxadiazol-2-yl)chroman-4- one |
| 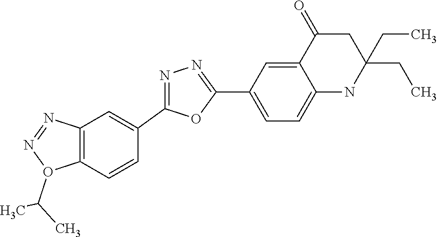 | 29 | 2,2-diethyl-6-(3-(1-isopropyl-1H- benzo[d][1,2,3]triazol-5-yl)-1,2,4- thiadiazol-5-yl)chroman-4-one |
| 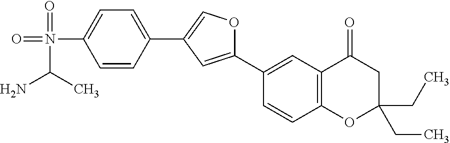 | 30 | 4-(5-(2,2-diethyl-4-oxochroman- 6-yl)-1,2,4-oxadiazol-3-yl)-N,N- dimethylbenzenesulfonamide |
| 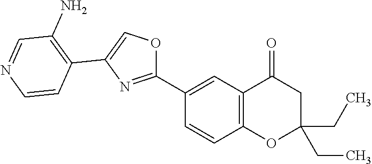 [SMILES: CCC1(CC)CC(=O)c2cc(-c3nc(-c4ccncc4N)co3)ccc2O1] | 31 | 2,2-diethyl-6-(3-(3- methylpyridin-4-yl)-1,2,4- oxadiazol-5-yl)chroman-4-one |
| 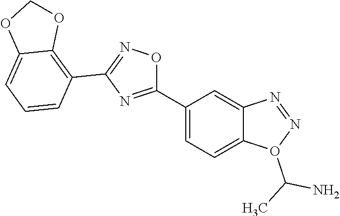 | 32 | 3-(benzo[d][1,3]dioxol-4-yl)-5-(1- isopropyl-1H- benzo[d][1,2,3]triazol-5-yl)-1,2,4- oxadiazole |
| 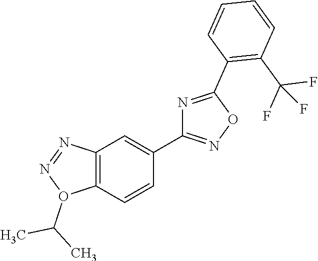 | 33 | 3-(1-isopropylbenzotriazol-5-yl)- 5-[2-(trifluoromethyl)phenyl]- 1,2,4-oxadiazole |
| 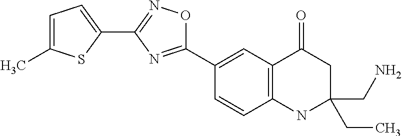 [SMILES: CCC1(CN)CC(=O)c2cc(-c3nc(-c4ccc(C)s4)no3)ccc2N1] | 34 | 2,2-diethyl-6-(3-(5- methylthiophen-2-yl)-1,2,4- oxadiazol-5-yl)chroman-4-one |
| 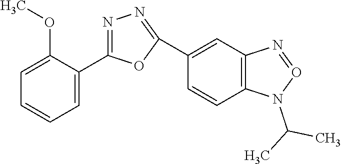 | 35 | 5-[5-(2-methoxyphenyl)-1,3,4- oxadiazol-2-yl]-1-(propan-2-yl)- 1H-1,2,3-benzotriazole |
| 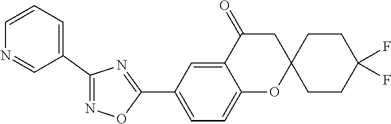 [SMILES: O=C1CC2(CCC(F)(F)CC2)Oc2ccc(-c3nc(-c4cccnc4)no3)cc21] | 36 | 4′,4′-difluoro-6-[3-(pyridin-3-yl)- 1,2,4-oxadiazol-5-yl]-3,4- dihydrospiro[1-benzopyran-2,1′- cyclohexane]-4-one |
| 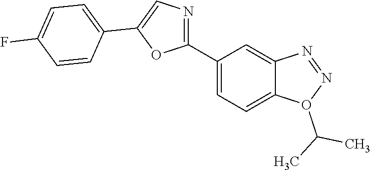 | 37 | 5-(4-fluorophenyl)-2-(1- isopropylbenzotriazol-5- yl)thiazole |
| 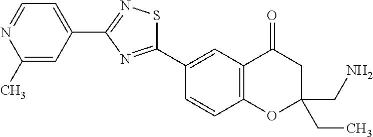 [SMILES: CCC1(CN)CC(=O)c2cc(-c3nc(-c4ccnc(C)c4)ns3)ccc2O1] | 38 | 2,2-diethyl-6-(3-(2- methoxypyridin-4-yl)-1,2,4- oxadiazol-5-yl)chroman-4-one |
| 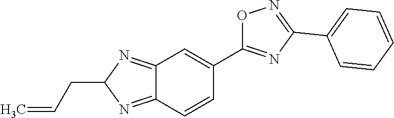 [SMILES: C=CCC1N=c2ccc(-c3nc(-c4ccccc4)no3)cc2=N1] | 39 | 5-(3-phenyl-1,2,4-oxadiazol-5- yl)-2-(prop-2-en-1-yl)-2H-1,2,3- benzotriazole |
| 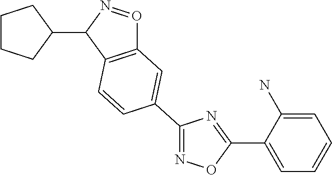 | 40 | 5-(2-bromophenyl)-3-(1- cyclopentyl-1H- benzo[d][1,2,3]triazol-5-yl)-1,2,4- oxadiazole |
| 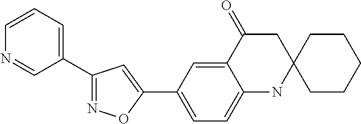 [SMILES: O=C1CC2(CCCCC2)Nc2ccc(-c3cc(-c4cccnc4)no3)cc21] | 41 | 6-[3-(pyridin-3-yl)-1,2,4- oxadiazol-5-yl]-3,4- dihydrospiro[1-benzopyran-2,1′- cyclohexane]-4-one |
| 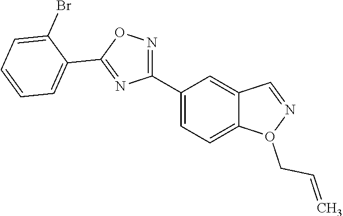 | 42 | 3-(1-allyl-1H- benzo[d][1,2,3]triazol-5-yl)-5-(2- bromophenyl)-1,2,4-oxadiazole |
| 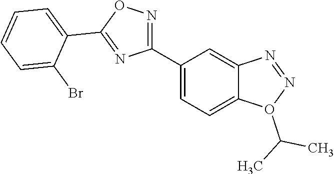 | 43 | 5-(2-bromophenyl)-3-(1- isopropyl-1H- benzo[d][1,2,3]triazol-5-yl)-1,2,4- oxadiazole |
| 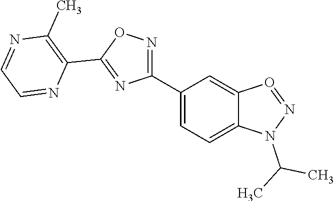 | 44 | 3-(1-isopropyl-1H- benzo[d][1,2,3]triazol-5-yl)-5-(3- methylpyrazin-2-yl)-1,2,4- oxadiazole |
| 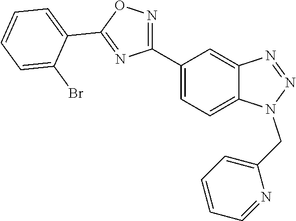 [SMILES: Brc1ccccc1-c1nc(-c2ccc3c(c2)nnn3Cc2ccccn2)no1] | 45 | 5-(2-bromophenyl)-3-(1-(pyridin- 2-ylmethyl)-1H- benzo[d][1,2,3]triazol-5-yl)-1,2,4- oxadiazole |
| 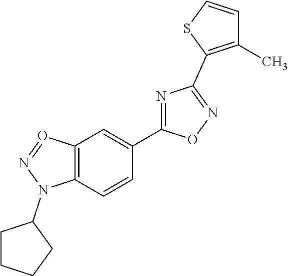 | 46 | 1-cyclopentyl-5-[3-(3- methylthiophen-2-yl)-1,2,4- oxadiazol-5-yl]-1H-1,2,3- benzotriazole |
| 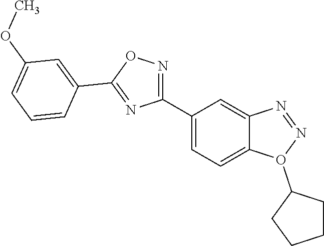 | 47 | 3-(1-cyclopentyl-1H- benzo[d][1,2,3]triazol-5-yl)-5-(3- (methylthio)phenyl)-1,2,4- oxadiazole |
| 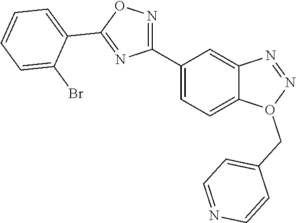 | 48 | 5-(2-bromophenyl)-3-(1-(pyridin- 4-ylmethyl)-1H- benzo[d][1,2,3]triazol-5-yl)-1,2,4- oxadiazole |
| 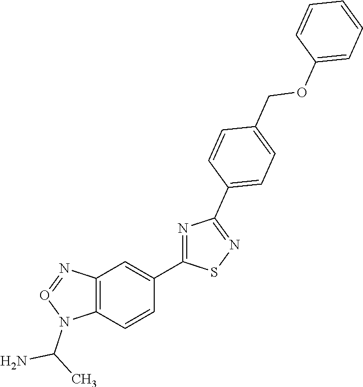 | 49 | 5-{3-[4-(phenoxymethyl)phenyl]- 1,2,4-oxadiazol-5-yl}-1-(propan- 2-yl)-1H-1,2,3-benzotriazole |
| 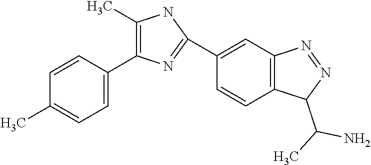 [SMILES: Cc1ccc(-c2nc(-c3ccc4c(c3)N=NC4C(C)N)[nH]c2C)cc1] | 50 | 5-[4-(4-chlorophenyl)-5-methyl- 1,3-oxazol-2-yl]-1-(propan-2-yl)- 1H-1,2,3-benzotriazole |
| 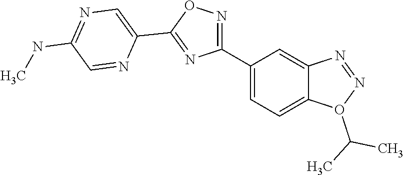 | 51 | 3-(1-isopropyl-1H- benzo[d][1,2,3]triazol-5-yl)-5-(5- methoxypyrazin-2-yl)-1,2,4- oxadiazole |
| 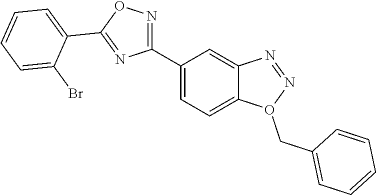 | 52 | 3-(1-benzyl-1H- benzo[d][1,2,3]triazol-5-yl)-5-(2- bromophenyl)-1,2,4-oxadiazole |
| 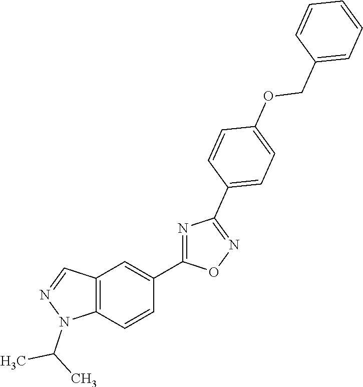 [SMILES: CC(C)n1ncc2cc(-c3nc(-c4ccc(OCc5ccccc5)cc4)no3)ccc21] | 53 | 5-{3-[4-(benzyloxy)phenyl]- 1,2,4-oxadiazol-5-yl}-1-(propan- 2-yl)-1H-1,2,3-benzotriazole |
| 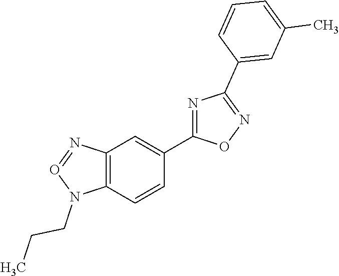 | 54 | 5-[3-(3-methylphenyl)-1,2,4- oxadiazol-5-yl]-1-propyl-1H- 1,2,3-benzotriazole |
| 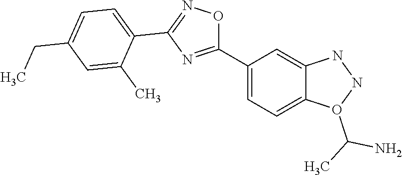 | 55 | 5-(1-isopropyl-1H- benzo[d][1,2,3]triazol-5-yl)-3-(4- methoxy-2-methylphenyl)-1,2,4- oxadiazole |
| 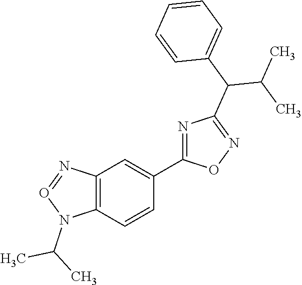 | 56 | 5-[3-(2-methyl-1-phenylpropyl)- 1,2,4-oxadiazol-5-yl]-1-(propan- 2-yl)-1H-1,2,3-benzotriazole |
| 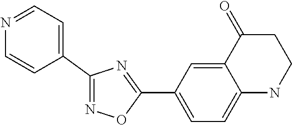 [SMILES: O=C1CCNc2ccc(-c3nc(-c4ccncc4)no3)cc21] | 57 | 6-[3-(pyridin-4-yl)-1,2,4- oxadiazol-5-yl]-3,4-dihydro-2H- 1-benzopyran-4-one |
| 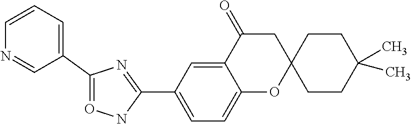 | 58 | 4′,4′-dimethyl-6-[3-(pyridin-3-yl)- 1,2,4-oxadiazol-5-yl]-3,4- dihydrospiro[1-benzopyran-2,1′- cyclohexane]-4-one |
| 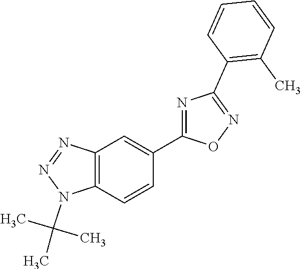 [SMILES: Cc1ccccc1-c1noc(-c2ccc3c(c2)nnn3C(C)(C)C)n1] | 59 | 1-tert-butyl-5-[3-(2- methylphenyl)-1,2,4-oxadiazol-5- yl]-1H-1,2,3-benzotriazole |
| 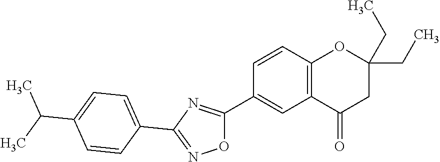 [SMILES: CCC1(CC)CC(=O)c2cc(-c3nc(-c4ccc(C(C)C)cc4)no3)ccc2O1] | 60 | 6-(3-(4-(dimethylamino)phenyl)- 1,2,4-oxadiazol-5-yl)-2,2- diethylchroman-4-one |
| 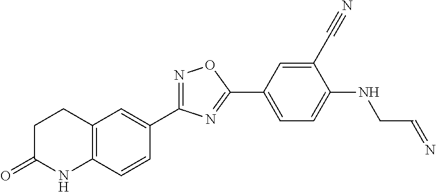 [SMILES: N#Cc1cc(-c2nc(-c3ccc4c(c3)CCC(=O)N4)no2)ccc1NCC=N] | 61 | 2-(allylamino)-5-(3-(2-oxo- 1,2,3,4-tetrahydroquinolin-6-yl)- 1,2,4-oxadiazol-5-yl)benzonitrile |
| 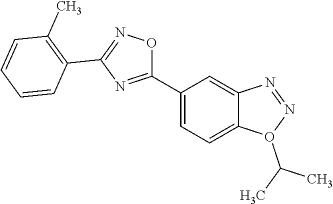 | 62 | 3-(2-isopropoxyphenyl)-5-(1- isopropyl-1H- benzo[d][1,2,3]triazol-5-yl)-1,2,4- oxadiazole |
| 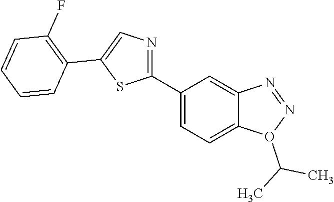 | 63 | 5-(2-fluorophenyl)-2-(1- isopropyl-1H- benzo[d][1,2,3]triazol-5- yl)thiazole |
| 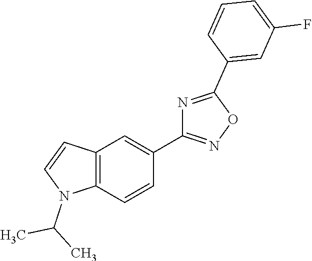 [SMILES: CC(C)n1ccc2cc(-c3noc(-c4cccc(F)c4)n3)ccc21] | 64 | 5-(3-fluorophenyl)-3-(1- isopropylbenzotriazol-5-yl)-1,2,4- oxadiazole |
| 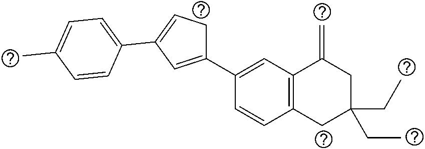 [SMILES: C=C1CC(CC)(CC)Cc2ccc(C3=CC(c4ccc(C)cc4)=CC3)cc21] | 65 | 2,2-diethyl-6-(3-(4- methoxyphenyl)-1,2,4-oxadiazol- 5-yl)chroman-4-one |
| 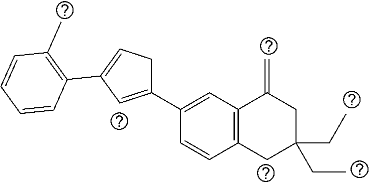 [SMILES: C=C1CC(CC)(CC)Cc2ccc(C3=CC(c4ccccc4C)=CC3)cc21] | 66 | 2,2-diethyl-6-(3-(o-tolyl)-1,2,4- oxadiazol-5-yl)chroman-4-one |
| 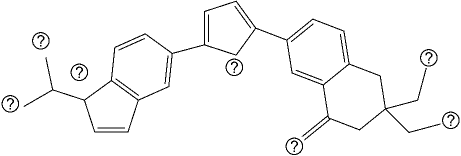 [SMILES: C=C1CC(CC)(CC)Cc2ccc(C3=CC=C(c4ccc5c(c4)C=CC5C(C)C)C3)cc21] | 67 | 2,2-diethyl-6-(5-(1-isopropyl-1H- benzo[d][1,2,3]triazol-5-yl)-1,3,4- thiadiazol-2-yl)chroman-4-one |
| 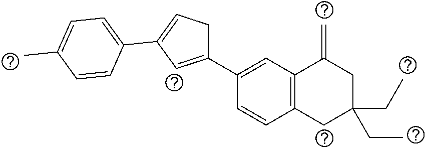 [SMILES: C=C1CC(CC)(CC)Cc2ccc(C3=CC(c4ccc(C)cc4)=CC3)cc21] | 68 | 6-[3-(4-chlorophenyl)-1,2,4- oxadiazol-5-yl]-2,2-diethyl- chroman-4-one |
| 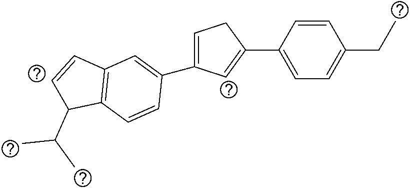 [SMILES: CCc1ccc(C2=CC(c3ccc4c(c3)C=CC4C(C)C)=CC2)cc1] | 69 | 3-(1-isopropylbenzotriazol-5-yl)- 5-(4-methoxyphenyl)-1,2,4- oxadiazole |
| 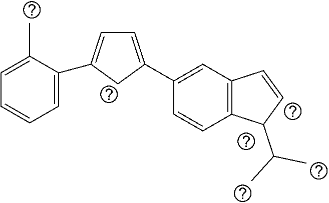 [SMILES: Cc1ccccc1C1=CC=C(c2ccc3c(c2)C=CC3C(C)C)C1] | 70 | 2-{5-[1-(propan-2-yl)-1H-1,2,3- benzotriazol-5-yl]-1,3,4- thiadiazol-2-yl}phenol |
| 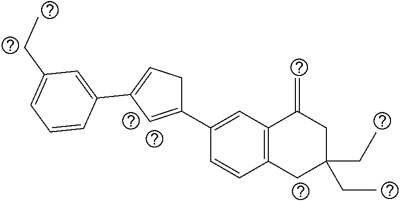 [SMILES: C=C1CC(CC)(CC)Cc2ccc(C3=CC(c4cccc(CC)c4)=CC3)cc21] | 71 | 2,2-diethyl-6-(3-(3- methoxyphenyl)-1,2,4-oxadiazol- 5-yl)chroman-4-one |
| 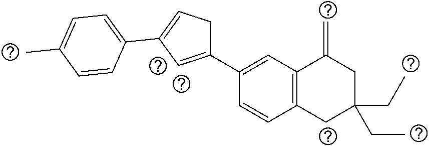 [SMILES: C=C1CC(CC)(CC)Cc2ccc(C3=CC(c4ccc(C)cc4)=CC3)cc21] | 72 | 2,2-diethyl-6-[3-(4- hydroxyphenyl)-1,2,4-oxadiazol- 5-yl]chroman-4-one |
| 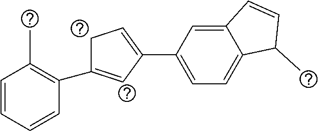 [SMILES: Cc1ccccc1C1=CC(c2ccc3c(c2)C=CC3C)=CC1] | 73 | 5-(2-bromophenyl)-3-(1-methyl- 1H-benzo[d][1,2,3]triazol-5-yl)- 1,2,4-oxadiazole |
| 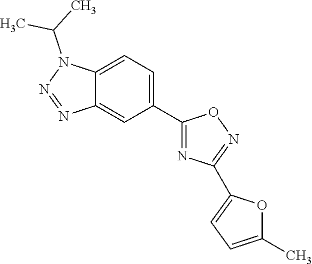 [SMILES: Cc1ccc(-c2noc(-c3ccc4c(c3)nnn4C(C)C)n2)o1] | 74 | 5-[5-(5-methylthiophen-2-yl)- 1,2,4-oxadiazol-3-yl]-1-(propan- 2-yl)-1H-1,2,3-benzotriazole |
| 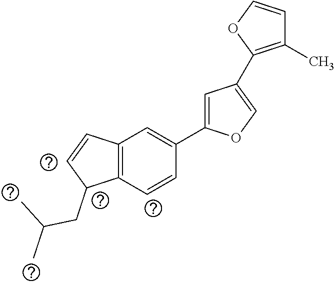 [SMILES: Cc1ccoc1-c1coc(-c2ccc3c(c2)C=CC3CC(C)C)c1] | 75 | 1-(2-methylpropyl)-5-[3-(3- methylthiophen-2-yl)-1,2,4- oxadiazol-5-yl]-1H-1,2,3- benzotriazole |
| 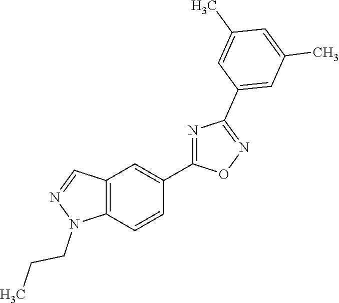 [SMILES: CCCn1ncc2cc(-c3nc(-c4cc(C)cc(C)c4)no3)ccc21] | 76 | 5-[3-(3,5-dimethylphenyl)-1,2,4- oxadiazol-5-yl]-1-propyl-1H- 1,2,3-benzotriazole |
| 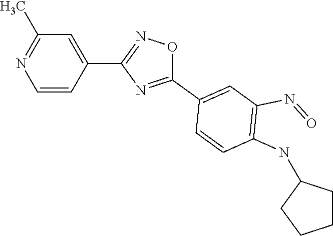 [SMILES: Cc1cc(-c2noc(-c3ccc(NC4CCCC4)c(N=O)c3)n2)ccn1] | 77 | 5-[3-(2-methoxypyridin-4-yl)- 1,2,4-oxadiazol-5-yl]-1-methyl- 2,3-dihydro-1H-1,2,3- benzotriazole; cyclopentane |
| 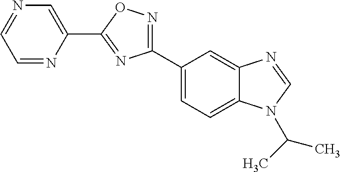 [SMILES: CC(C)n1cnc2cc(-c3noc(-c4cnccn4)n3)ccc21] | 78 | 3-(1-isopropyl-1H- benzo[d][1,2,3]triazol-5-yl)-5- (pyrazin-2-yl)-1,2,4-oxadiazole |
| 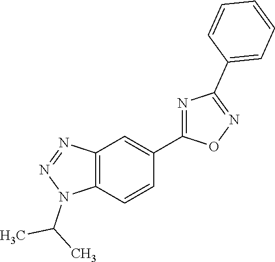 [SMILES: CC(C)n1nnc2cc(-c3nc(-c4ccccc4)no3)ccc21] | 79 | 5-(3-phenyl-1,2,4-oxadiazol-5- yl)-1-(propan-2-yl)-1H-1,2,3- benzotriazole |
| 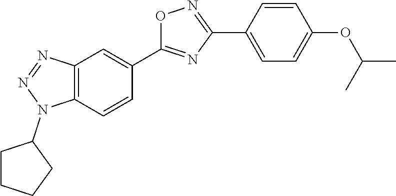 [SMILES: CC(C)Oc1ccc(-c2noc(-c3ccc4c(c3)nnn4C3CCCC3)n2)cc1] | 80 | 1-cyclopentyl-5-{3-[4- (difluoromethoxy)phenyl]-1,2,4- oxadiazol-5-yl}-1H-1,2,3- benzotriazole |
| 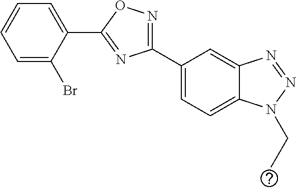 [SMILES: CCn1nnc2cc(-c3noc(-c4ccccc4Br)n3)ccc21] | 81 | methyl 2-(5-(5-(2-bromophenyl)- 1,2,4-oxadiazol-3-yl)-1H- benzo[d][1,2,3]triazol-1-yl)acetate |
| 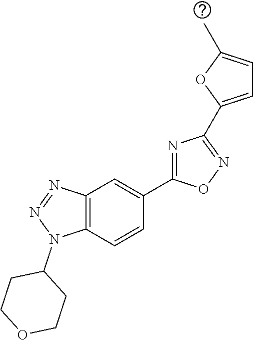 [SMILES: Cc1ccc(-c2noc(-c3ccc4c(c3)nnn4C3CCOCC3)n2)o1] | 82 | 5-[3-(5-chlorothiophen-2-yl)- 1,2,4-oxadiazol-5-yl]-1-(oxan-4- yl)-1H-1,2,3-benzotriazole |
| 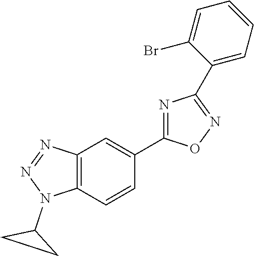 [SMILES: Brc1ccccc1-c1noc(-c2ccc3c(c2)nnn3C2CC2)n1] | 83 | 5-[3-(2-bromophenyl)-1,2,4- oxadiazol-5-yl]-1-cyclopropyl- 1H-1,2,3-benzotriazole |
| 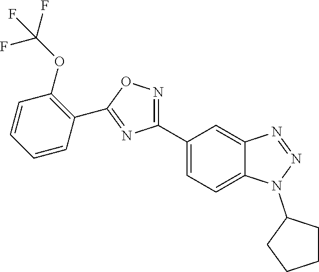 [SMILES: FC(F)(F)Oc1ccccc1-c1nc(-c2ccc3c(c2)nnn3C2CCCC2)no1] | 84 | 3-(1-cyclopentyl-1H- benzo[d][1,2,3]triazol-5-yl)-5-(2- (trifluoromethoxy)phenyl)-1,2,4- oxadiazole |
| 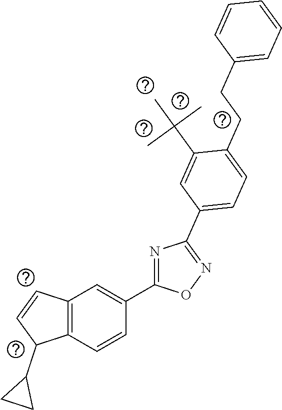 [SMILES: CC(C)(C)c1cc(-c2noc(-c3ccc4c(c3)C=CC4C3CC3)n2)ccc1CCc1ccccc1] | 85 | 5-{3-[4-(benzyloxy)-3- (trifluoromethyl)phenyl]-1,2,4- oxadiazol-5-yl}-1-cyclopropyl- 1H-1,2,3-benzotriazole |
| 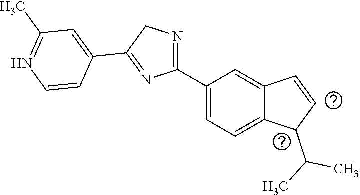 [SMILES: Cc1cc(C2=NC(c3ccc4c(c3)C=CC4C(C)C)=NC2)ccn1] | 86 | 3-(1-isopropyl-1H- benzo[d][1,2,3]triazol-5-yl)-5-(2- methylpyridin-4-yl)-1,2,4- oxadiazole |
| 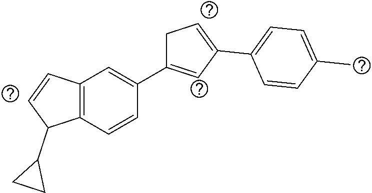 [SMILES: Cc1ccc(C2=CCC(c3ccc4c(c3)C=CC4C3CC3)=C2)cc1] | 87 | 1-cyclopropyl-5-[3-(6- methylpyridin-3-yl)-1,2,4- oxadiazol-5-yl]-1H-1,2,3- benzotriazole |
| 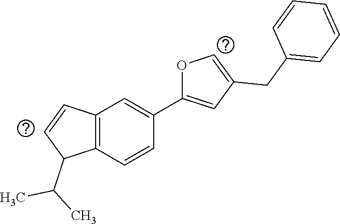 [SMILES: CC(C)C1C=Cc2cc(-c3cc(Cc4ccccc4)co3)ccc21] | 88 | 5-(3-benzyl-1,2,4-oxadiazol-5-yl)- 1-(propan-2-yl)-1H-1,2,3- benzotriazole |
| 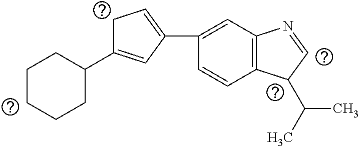 [SMILES: CC(C)C1C=Nc2cc(C3=CCC(C4CCCCC4)=C3)ccc21] | 89 | 5-[5-(oxan-4-yl)-1,2,4-oxadiazol- 3-yl]-1-(propan-2-yl)-1H-1,2,3- benzotriazole |
| 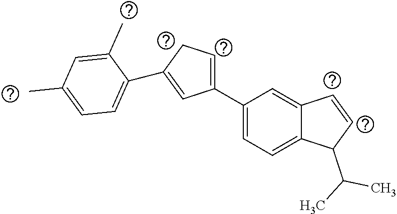 [SMILES: Cc1ccc(C2=CC(c3ccc4c(c3)C=CC4C(C)C)=CC2)c(C)c1] | 90 | 3-(1-isopropyl-1H- benzo[d][1,2,3]triazol-5-yl)-5-(4- methoxy-2-methylphenyl)-1,2,4- oxadiazole |
| 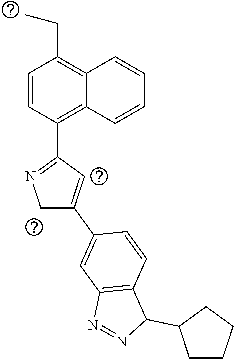 [SMILES: CCc1ccc(C2=NCC(c3ccc4c(c3)N=NC4C3CCCC3)=C2)c2ccccc12] | 91 | 1-cyclopentyl-5-[3-(4- methoxynaphthalen-1-yl)-1,2,4- oxadiazol-5-yl]-1H-1,2,3- benzotriazole |
| 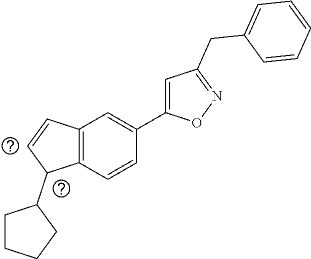 [SMILES: C1=CC(C2CCCC2)c2ccc(-c3cc(Cc4ccccc4)no3)cc21] | 92 | 5-(3-benzyl-1,2,4-oxadiazol-5-yl)- 1-cyclopentyl-1H-1,2,3- benzotriazole |
| 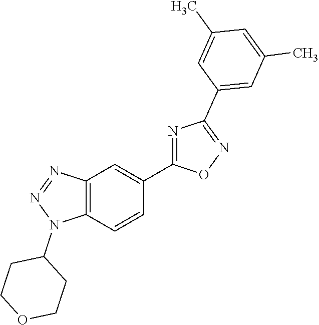 [SMILES: Cc1cc(C)cc(-c2noc(-c3ccc4c(c3)nnn4C3CCOCC3)n2)c1] | 93 | 5-[3-(3,5-dimethylphenyl)-1,2,4- oxadiazol-5-yl]-1-(oxan-4-yl)- 1H-1,2,3-benzotriazole |
| 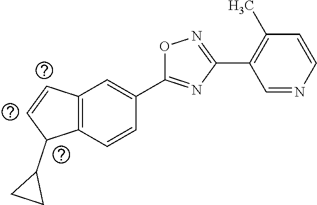 [SMILES: Cc1ccncc1-c1noc(-c2ccc3c(c2)C=CC3C2CC2)n1] | 94 | 1-cyclopropyl-5-[3-(4- methylpyridin-3-yl)-1,2,4- oxadiazol-5-yl]-1H-1,2,3- benzotriazole |
| 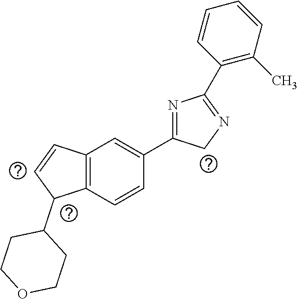 [SMILES: Cc1ccccc1C1=NCC(c2ccc3c(c2)C=CC3C2CCOCC2)=N1] | 95 | 5-[3-(2-methylphenyl)-1,2,4- oxadiazol-5-yl]-1-(oxan-4-yl)- 1H-1,2,3-benzotriazole |
| 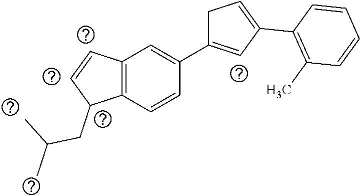 [SMILES: Cc1ccccc1C1=CCC(c2ccc3c(c2)C=CC3CC(C)C)=C1] | 96 | 5-[3-(2-methylphenyl)-1,2,4- oxadiazol-5-yl]-1-(2- methylpropyl)-1H-1,2,3- benzotriazole |
| 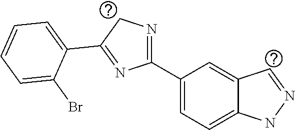 [SMILES: Brc1ccccc1C1=NC(c2ccc3[nH]ncc3c2)=NC1] | 97 | 3-(1H-benzo[d][1,2,3]triazol-5- yl)-5-(2-bromophenyl)-1,2,4- oxadiazole |
| 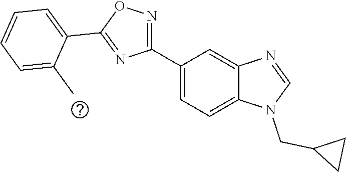 [SMILES: Cc1ccccc1-c1nc(-c2ccc3c(c2)ncn3CC2CC2)no1] | 98 | 5-(2-bromophenyl)-3-(1- (cyclopropylmethvl)-1H- benzo[d][1,2,3]triazol-5-yl)-1,2,4- oxadiazole |
| 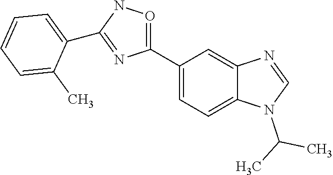 | 99 | 3-(1-isopropyl-1H- benzo[d][1,2,3]triazol-5-yl)-5-(o- tolyl)-1,2,4-oxadiazole |
| 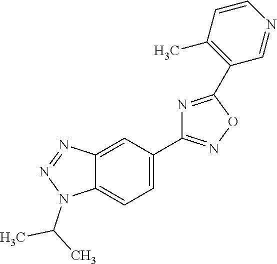 [SMILES: Cc1ccncc1-c1nc(-c2ccc3c(c2)nnn3C(C)C)no1] | 100 | 3-(1-isopropyl-1H- benzo[d][1,2,3]triazol-5-yl)-5-(4- methylpyridin-3-yl)-1,2,4- oxadiazole |
| 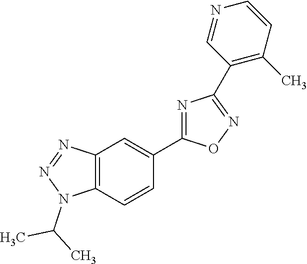 [SMILES: Cc1ccncc1-c1noc(-c2ccc3c(c2)nnn3C(C)C)n1] | 101 | 5-[3-(4-methylpyridin-3-yl)-1,2,4- oxadiazol-5-yl]-1-(propan-2-yl)- 1H-1,2,3-benzotriazole |
| 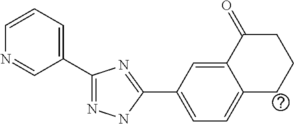 [SMILES: O=C1CCCc2ccc(-c3nc(-c4cccnc4)n[nH]3)cc21] | 102 | 6-[3-(pyridin-3-yl)-1,2,4- oxadiazol-5-yl]-3,4-dihydro-2H- 1-benzopyran-4-one |
| 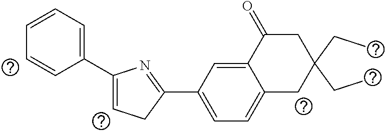 [SMILES: CCC1(CC)CC(=O)c2cc(C3=NC(c4ccccc4)=CC3)ccc2C1] | 103 | 2,2-diethyl-6-[3-(pyridin-3-yl)- 1,2,4-oxadiazol-5-yl]-3,4- dihydro-2H-1-benzopyran-4-one |
| 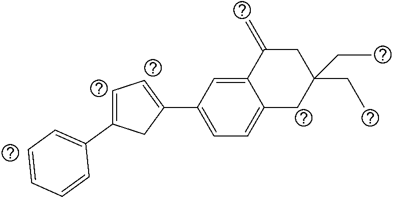 [SMILES: C=C1CC(CC)(CC)Cc2ccc(C3=CC=C(c4ccccc4)C3)cc21] | 104 | 2,2-diethyl-6-(5-(pyridin-3-yl)- 1,3,4-thiadiazol-2-yl)chroman-4- one |
| 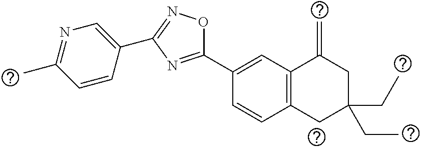 [SMILES: C=C1CC(CC)(CC)Cc2ccc(-c3nc(-c4ccc(C)nc4)no3)cc21] | 105 | 2,2-diethyl-6-[3-(6-hydroxy-3- pyridyl)-1,2,4-oxadiazol-5- yl]chroman-4-one |
| 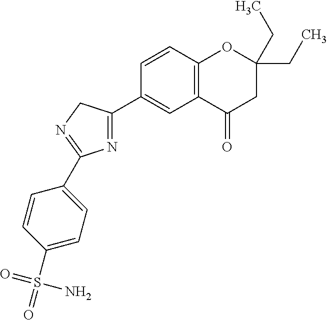 [SMILES: CCC1(CC)CC(=O)c2cc(C3=NC(c4ccc(S(N)(=O)=O)cc4)=NC3)ccc2O1] | 106 | 4-(5-(2,2-diethyl-4-oxochroman- 6-yl)-1,2,4-oxadiazol-3- yl)benzenesulfonamide |
| 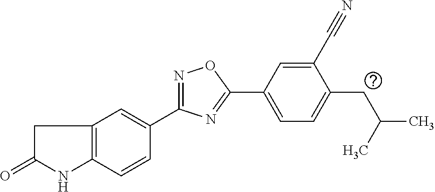 [SMILES: CC(C)Cc1ccc(-c2nc(-c3ccc4c(c3)CC(=O)N4)no2)cc1C#N] | 107 | 2-(isopropylamino)-5-(3-(2- oxoindolin-5-yl)-1,2,4-oxadiazol- 5-yl)benzonitrile |
| 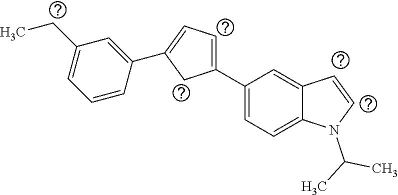 [SMILES: CCc1cccc(C2=CC=C(c3ccc4c(ccn4C(C)C)c3)C2)c1] | 108 | 2-(1-isopropyl-1H- benzo[d][1,2,3]triazol-5-yl)-5-(3- methoxyphenyl)thiazole |
| 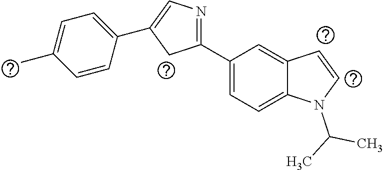 [SMILES: Cc1ccc(C2=CN=C(c3ccc4c(ccn4C(C)C)c3)C2)cc1] | 109 | 2-(1-isopropyl-1H- benzo[d][1,2,3]triazol-5-yl)-5-(4- methoxyphenyl)thiazole |
| 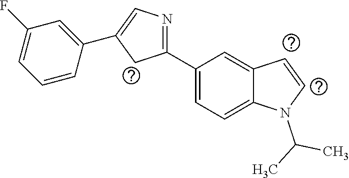 [SMILES: CC(C)n1ccc2cc(C3=NC=C(c4cccc(F)c4)C3)ccc21] | 110 | 5-(3-fluorophenyl)-2-(1- isopropyl-1H- benzo[d][1,2,3]triazol-5- yl)thiazole |
| 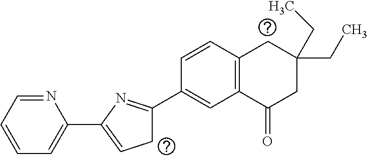 [SMILES: CCC1(CC)CC(=O)c2cc(C3=NC(c4ccccn4)=CC3)ccc2C1] | 111 | 2,2-diethyl-6-[3-(2-pyridyl)-1,2,4- oxadiazol-5-yl]chroman-4-one |
| 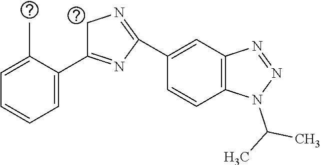 [SMILES: Cc1ccccc1C1=NC(c2ccc3c(c2)nnn3C(C)C)=NC1] | 112 | 2-{3-[1-(propan-2-yl)-1H-1,2,3- benzotriazol-5-yl]-1,2,4- thiadiazol-5-yl}phenol |
| 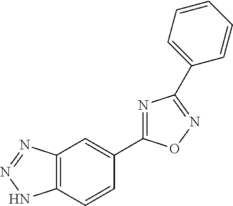 [SMILES: c1ccc(-c2noc(-c3ccc4[nH]nnc4c3)n2)cc1] | 113 | 5-(3-phenyl-1,2,4-oxadiazol-5- yl)-1H-1,2,3-benzotriazole |
| 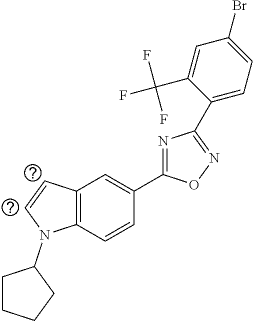 [SMILES: FC(F)(F)c1cc(Br)ccc1-c1noc(-c2ccc3c(ccn3C3CCCC3)c2)n1] | 114 | 5-{3-[4-bromo-2- (trifluoromethyl)phenyl]-1,2,4- oxadiazol-5-yl}-1-cyclopentyl- 1H-1,2,3-benzotriazole |
| 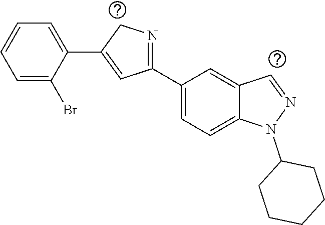 [SMILES: Brc1ccccc1C1=CC(c2ccc3c(cnn3C3CCCCC3)c2)=NC1] | 115 | 5-(2-bromophenyl)-3-(1- cyclohexyl-1H- benzo[d][1,2,3]triazol-5-yl)-1,2,4- oxadiazole |
| 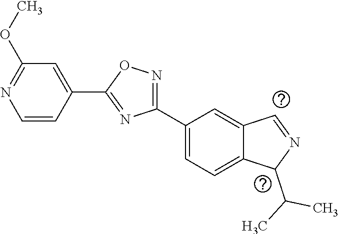 [SMILES: COc1cc(-c2nc(-c3ccc4c(c3)C=NC4C(C)C)no2)ccn1] | 116 | 3-(1-isopropyl-1H- benzo[d][1,2,3]triazol-5-yl)-5-(2- methoxypyridin-4-yl)-1,2,4- oxadiazole |
| 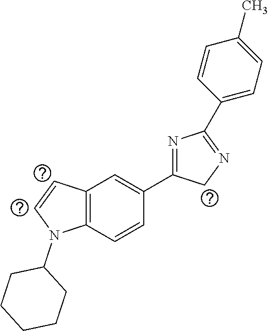 [SMILES: Cc1ccc(C2=NCC(c3ccc4c(ccn4C4CCCCC4)c3)=N2)cc1] | 117 | 1-cyclohexyl-5-[3-(4- methylphenyl)-1,2,4-oxadiazol-5- yl]-1H-1,2,3-benzotriazole |
| 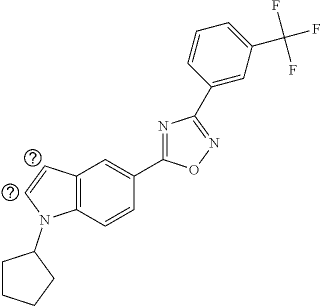 [SMILES: FC(F)(F)c1cccc(-c2noc(-c3ccc4c(ccn4C4CCCC4)c3)n2)c1] | 118 | 1-cyclopentyl-5-{3-[3- (trifluoromethyl)phenyl]-1,2,4- oxadiazol-5-yl}-1H-1,2,3- benzotriazole |
| 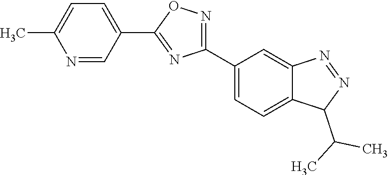 [SMILES: Cc1ccc(-c2nc(-c3ccc4c(c3)N=NC4C(C)C)no2)cn1] | 119 | 3-(1-isopropyl-1H- benzo[d][1,2,3]triazol-5-yl)-5-(6- methylpyridin-3-yl)-1,2,4- oxadiazole |
| 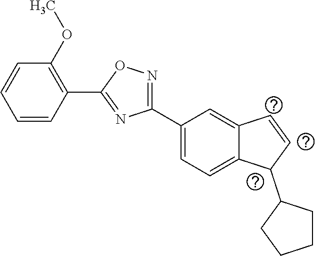 [SMILES: COc1ccccc1-c1nc(-c2ccc3c(c2)C=CC3C2CCCC2)no1] | 120 | 3-(1-cyclopentyl-1H- benzo[d][1,2,3]triazol-5-yl)-5-(2- methoxyphenyl)-1,2,4-oxadiazole |
| 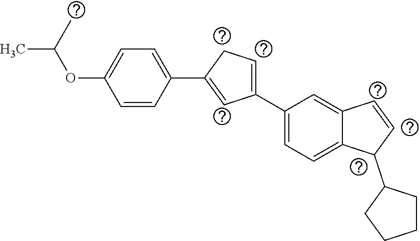 [SMILES: CC(C)Oc1ccc(C2=CC(c3ccc4c(c3)C=CC4C3CCCC3)=CC2)cc1] | 121 | 3-(1-cyclopentyl-1H- benzo[d][1,2,3]triazol-5-yl)-5-(4- isopropoxyphenyl)-1,2,4- oxadiazole |
| 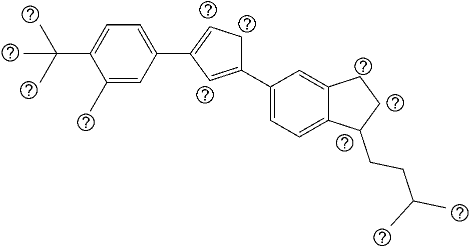 [SMILES: Cc1cc(C2=CCC(c3ccc4c(c3)CCC4CCC(C)C)=C2)ccc1C(C)(C)C] | 122 | 5-{3-[3-fluoro-4- (trifluoromethyl)phenyl]-1,2,4- oxadiazol-5-yl}-1-(3- methylbutyl)-1H-1,2,3- benzotriazole |
| 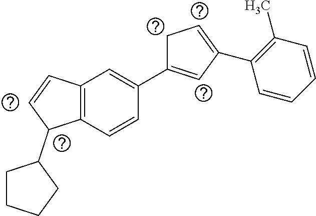 [SMILES: Cc1ccccc1C1=CCC(c2ccc3c(c2)C=CC3C2CCCC2)=C1] | 123 | 5-[3-(3,5-dimethylphenyl)-1,2,4- oxadiazol-5-yl]-1-(propan-2-yl)- 1H-1,2,3-benzotriazole |
| 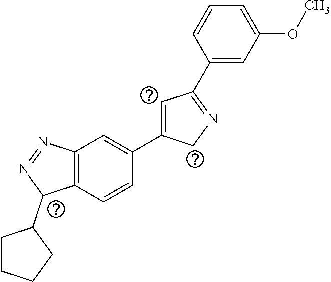 [SMILES: COc1cccc(C2=NCC(c3ccc4c(c3)N=NC4C3CCCC3)=C2)c1] | 124 | 1-cyclopentyl-5-[3-(6- methoxypyridin-2-yl)-1,2,4- oxadiazol-5-yl]-1H-1,2,3- benzotriazole |
| 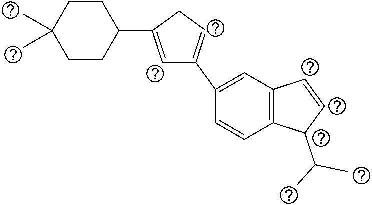 [SMILES: CC(C)C1C=Cc2cc(C3=CCC(C4CCC(C)(C)CC4)=C3)ccc21] | 125 | 5-[5-(4,4-difluorocyclohexyl)- 1,2,4-oxadiazol-3-yl]-1-(propan- 2-yl)-1H-1,2,3-benzotriazole |
| 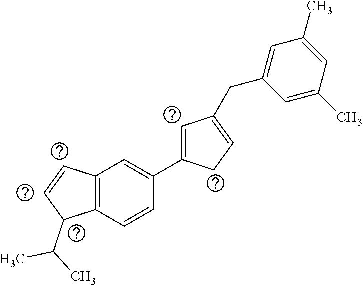 [SMILES: Cc1cc(C)cc(CC2=CCC(c3ccc4c(c3)C=CC4C(C)C)=C2)c1] | 126 | 5-{3-[(3,5- dimethylphenyl)methyl]-1,2,4- oxadiazol-5-yl}-1-(propan-2-yl)- 1H-1,2,3-benzotriazole |
| 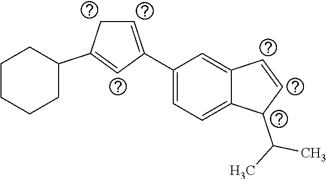 [SMILES: CC(C)C1C=Cc2cc(C3=CCC(C4CCCCC4)=C3)ccc21] | 127 | 5-(5-cyclohexyl-1,2,4-oxadiazol- 3-yl)-1-(propan-2-yl)-1H-1,2,3- benzotriazole |
| 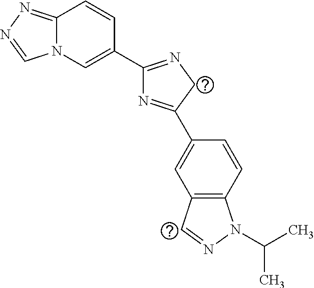 [SMILES: CC(C)n1ncc2cc(C3=NC(c4ccc5nncn5c4)=NC3)ccc21] | 128 | 1-(propan-2-yl)-5-(3- {[1,2,4]triazolo[4,3-a]pyridin-6- yl}-1,2,4-oxadiazol-5-yl)-1H- 1,2,3-benzotriazole |
| 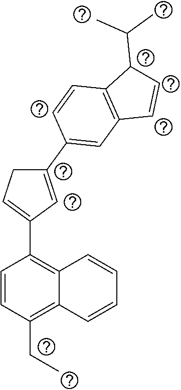 [SMILES: CCc1ccc(C2=CCC(c3ccc4c(c3)C=CC4C(C)C)=C2)c2ccccc12] | 129 | 5-[3-(4-methoxynaphthalen-1-yl)- 1,2,4-oxadiazol-5-yl]-1-(propan- 2-yl)-1H-1,2,3-benzotriazole |
| 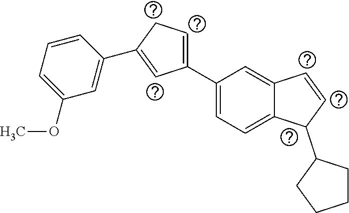 [SMILES: COc1cccc(C2=CC(c3ccc4c(c3)C=CC4C3CCCC3)=CC2)c1] | 130 | 3-(1-cyclopentyl-1H- benzo[d][1,2,3]triazol-5-yl)-5-(3- methoxyphenyl)-1,2,4-oxadiazole |
| 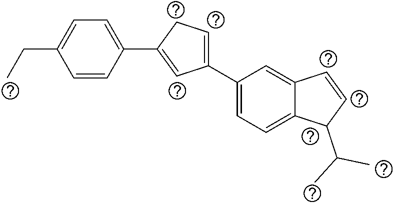 [SMILES: CCc1ccc(C2=CC(c3ccc4c(c3)C=CC4C(C)C)=CC2)cc1] | 131 | 3-(1-isopropyl-1H- benzo[d][1,2,3]triazol-5-yl)-5-(4- (phenoxymethyl)phenyl)-1,2,4- oxadiazole |
| 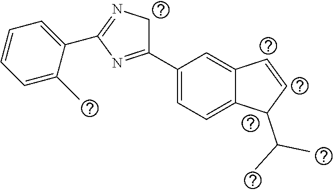 [SMILES: Cc1ccccc1C1=NCC(c2ccc3c(c2)C=CC3C(C)C)=N1] | 132 | 5-(1-isopropyl-1H- benzo[d][1,2,3]triazol-5-yl)-3-(2- (trifluoromethoxy)phenyl)-1,2,4- oxadiazole |
| 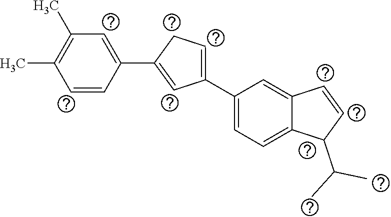 [SMILES: Cc1ccc(C2=CC(c3ccc4c(c3)C=CC4C(C)C)=CC2)cc1C] | 133 | 5-(5,6-dimethylpyrazin-2-yl)-3- (1-isopropyl-1H- benzo[d][1,2,3]triazol-5-yl)-1,2,4- oxadiazole |
| 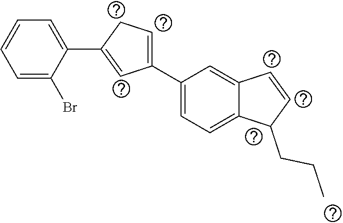 [SMILES: CCCC1C=Cc2cc(C3=CCC(c4ccccc4Br)=C3)ccc21] | 134 | 3-(5-(5-(2-bromophenyl)-1,2,4- oxadiazol-3-yl)-1H- benzo[d][1,2,3]triazol-1- yl)propanoic acid |
| 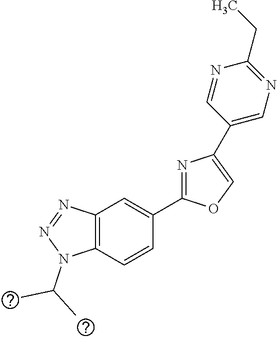 [SMILES: CCc1ncc(-c2coc(-c3ccc4c(c3)nnn4C(C)C)n2)cn1] | 135 | 5-[3-(2-ethylpyrimidin-5-yl)- 1,2,4-oxadiazol-5-yl]-1-(propan- 2-yl)-1H-1,2,3-benzotriazole |
| 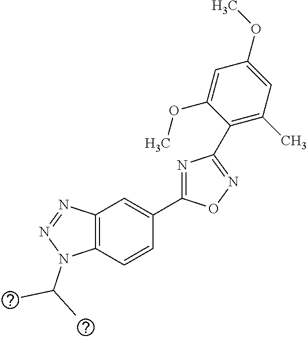 [SMILES: COc1cc(C)c(-c2noc(-c3ccc4c(c3)nnn4C(C)C)n2)c(OC)c1] | 136 | 5-[3-(2,4-dimethoxy-6- methylphenyl)-1,2,4-oxadiazol-5- yl]-1-(propan-2-yl)-1H-1,2,3- benzotriazole |
| 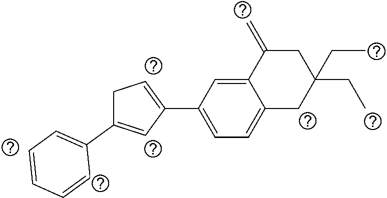 [SMILES: C=C1CC(CC)(CC)Cc2ccc(C3=CCC(c4ccccc4)=C3)cc21] | 137 | 2,2-diethyl-6-(5-(pyridin-3-yl)- 1,2,4-thiadiazol-3-yl)chroman-4- one |
| 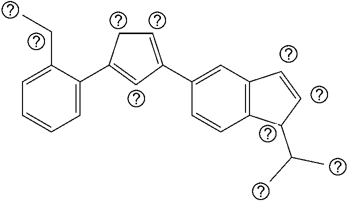 [SMILES: CCc1ccccc1C1=CC(c2ccc3c(c2)C=CC3C(C)C)=CC1] | 138 | 5-[5-(2-methoxyphenyl)-1,2,4- thiadiazol-3-yl]-1-(propan-2-yl)- 1H-1,2,3-benzotriazole |
| 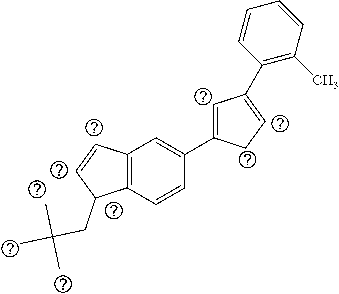 [SMILES: Cc1ccccc1C1=CCC(c2ccc3c(c2)C=CC3CC(C)(C)C)=C1] | 139 | 2-methyl-1-{5-[3-(2- methylphenyl)-1,2,4-oxadiazol-5- yl]-1H-1,2,3-benzotriazol-1- yl}propan-2-ol |
| 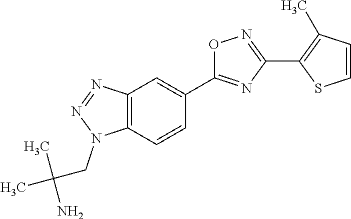 [SMILES: Cc1ccsc1-c1noc(-c2ccc3c(c2)nnn3CC(C)(C)N)n1] | 140 | 2-methyl-1-{5-[3-(3- methylthiophen-2-yl)-1,2,4- oxadiazol-5-yl]-1H-1,2,3- benzotriazol-1-yl}propan-2-ol |
| 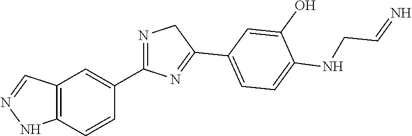 [SMILES: N=CCNc1ccc(C2=NC(c3ccc4[nH]ncc4c3)=NC2)cc1O] | 141 | 5-(3-(1H-indazol-5-yl)-1,2,4- oxadiazol-5-yl)-2- (allylamino)benzonitrile |
| 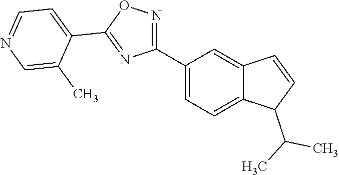 [SMILES: Cc1cnccc1-c1nc(-c2ccc3c(c2)C=CC3C(C)C)no1] | 142 | 3-(1-isopropyl-1H-indol-5-yl)-5- (3-methylpyridin-4-yl)-1,2,4- oxadiazole |
| 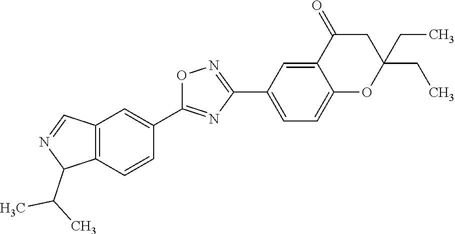 [SMILES: CCC1(CC)CC(=O)c2cc(-c3noc(-c4ccc5c(c4)C=NC5C(C)C)n3)ccc2O1] | 143 | 2,2-diethyl-6-(5-(l-isopropyl-1H- benzo[d][1,2,3]triazol-5-yl)-1,2,4- thiadiazol-3-yl)chroman-4-one |
| 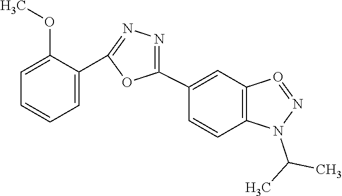 | 144 | 5-[5-(2-methoxyphenyl)-1,3,4- thiadiazol-2-yl]-1-(propan-2-yl)- 1H-1,2,3-benzotriazole |
| 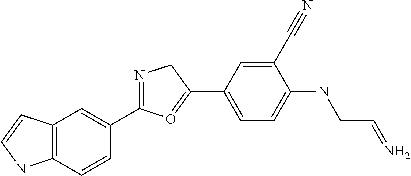 | 145 | 5-(3-(1H-benzo[d][1,2,3]triazol-5- yl)-1,2,4-oxadiazol-5-yl)-2- (allylamino)benzonitrile |
| 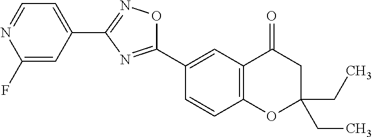 [SMILES: CCC1(CC)CC(=O)c2cc(-c3nc(-c4ccnc(F)c4)no3)ccc2O1] | 146 | 2,2-diethyl-6-(3-(2-fluoropyridin- 4-yl)-1,2,4-oxadiazol-5- yl)chroman-4-one |
| 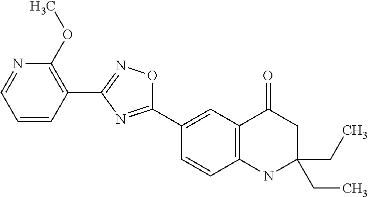 [SMILES: CCC1(CC)CC(=O)c2cc(-c3nc(-c4cccnc4OC)no3)ccc2N1] | 147 | 2,2-diethyl-6-(3-(2- methoxypyridin-3-yl)-1,2,4- oxadiazol-5-yl)chroman-4-one |
| 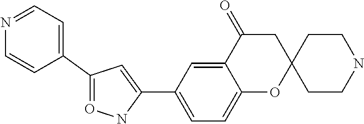 | 148 | 6-[3-(pyridin-4-yl)-1,2,4- oxadiazol-5-yl]-3,4- dihydrospiro[1-benzopyran-2,4′- oxane]-4-one |
| 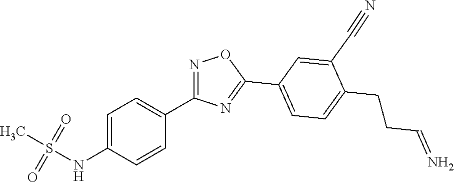 [SMILES: CS(=O)(=O)Nc1ccc(-c2noc(-c3ccc(CCC=N)c(C#N)c3)n2)cc1] | 149 | N-(4-(5-(4-(allylamino)-3- cyanophenyl)-1,2,4-oxadiazol-3- yl)phenyl)methanesulfonamide |
| 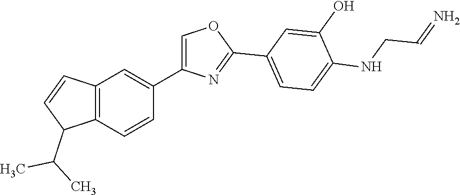 [SMILES: CC(C)C1C=Cc2cc(-c3coc(-c4ccc(NCC=N)c(O)c4)n3)ccc21] | 150 | 2-(allylamino)-5-(3-(1-isopropyl- 1H-benzo[d][1,2,3]triazol-5-yl)- 1,2,4-oxadiazol-5-yl)benzonitrile |
| 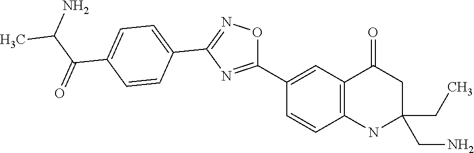 [SMILES: CCC1(CN)CC(=O)c2cc(-c3nc(-c4ccc(C(=O)C(C)N)cc4)no3)ccc2N1] | 151 | 4-(5-(2,2-diethyl-4-oxochroman- 6-yl)-1,2,4-oxadiazol-3-yl)-N,N- dimethylbenzamide |
| 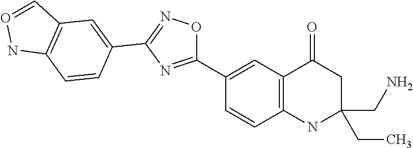 | 152 | 6-(3-(1H-indazol-5-yl)-1,2,4- oxadiazol-5-yl)-2,2- diethylchroman-4-one |
| 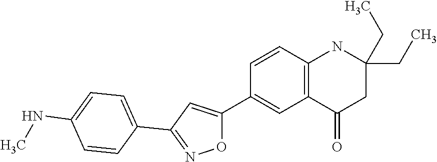 [SMILES: CCC1(CC)CC(=O)c2cc(-c3cc(-c4ccc(NC)cc4)no3)ccc2N1] | 153 | 2,2-diethyl-6-(3-(4- (methylamino)phenyl)-1,2,4- oxadiazol-5-yl)chroman-4-one |
| 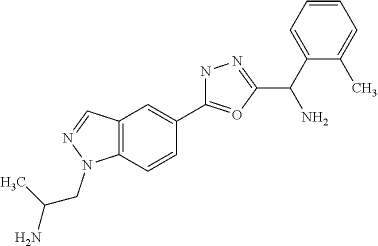 | 154 | 5-{3-[1-(2-methylphenyl)ethyl]- 1,2,4-oxadiazol-5-yl}-1-(2- methylpropyl)-1H-1,2,3- benzotriazole |
| 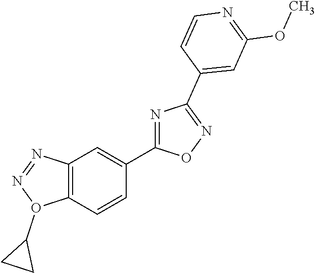 | 155 | 1-cyclopropyl-5-[3-(2- methoxypyridin-4-yl)-1,2,4- oxadiazol-5-yl]-1H-1,2,3- benzotriazole |
| 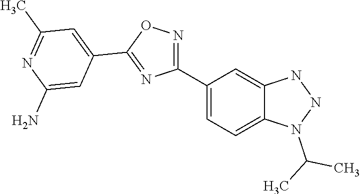 [SMILES: Cc1cc(-c2nc(-c3ccc4c(c3)NNN4C(C)C)no2)cc(N)n1] | 156 | 5-(2,6-dimethylpyridin-4-yl)-3-(1- isopropyl-1H- benzo[d][1,2,3]triazol-5-yl)-1,2,4- oxadiazole |
| 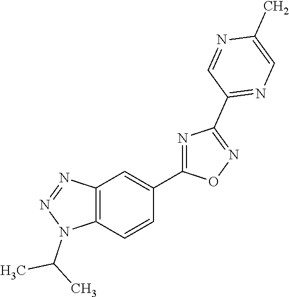 [SMILES: Cc1cnc(-c2noc(-c3ccc4c(c3)nnn4C(C)C)n2)cn1] | 157 | 5-[3-(5-methylpyrazin-2-yl)- 1,2,4-oxadiazol-5-yl]-1-(propan- 2-yl)-1H-1,2,3-benzotriazole |
| 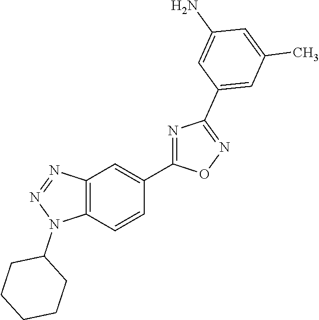 [SMILES: Cc1cc(N)cc(-c2noc(-c3ccc4c(c3)nnn4C3CCCCC3)n2)c1] | 158 | 1-cyclohexyl-5-[3-(3,5- dimethylphenyl)-1,2,4-oxadiazol- 5-yl]-1H-1,2,3-benzotriazole |
| 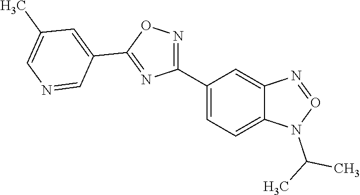 | 159 | 3-(1-isopropyl-1H- benzo[d][1,2,3]triazol-5-yl)-5-(5- methylpyridin-3-yl)-1,2,4- oxadiazole |
| 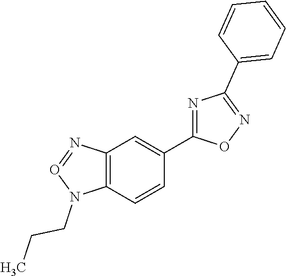 | 160 | 5-(3-phenyl-1,2,4-oxadiazol-5- yl)-1-propyl-1H-1,2,3- benzotriazole |
| 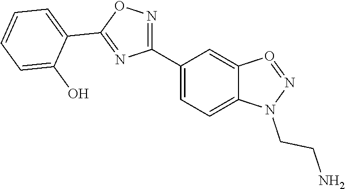 | 161 | methyl 3-(5-(5-(2-bromophenyl)- 1,2,4-oxadiazol-3-yl)-1H- benzo[d][1,2,3]triazol-1- yl)propanoate |
| 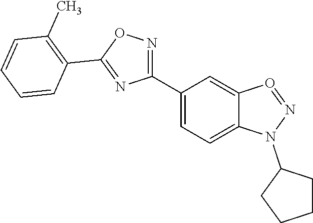 | 162 | 3-(1-cyclopentyl-1H- benzo[d][1,2,3]triazol-5-yl)-5-(o- tolyl)-1,2,4-oxadiazole |
| 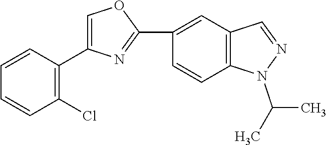 [SMILES: CC(C)n1ncc2cc(-c3nc(-c4ccccc4Cl)co3)ccc21] | 163 | 5-[4-(2-chlorophenyl)-2,3- dihydro-1,3-oxazol-2-yl]-1- (propan-2-yl)-1H-1,2,3- benzotriazole |
| 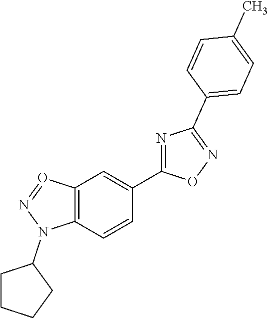 | 164 | 1-cyclopentyl-5-[3-(4- methylphenyl)-1,2,4-oxadiazol-5- yl]-1H-1,2,3-benzotriazole |
| 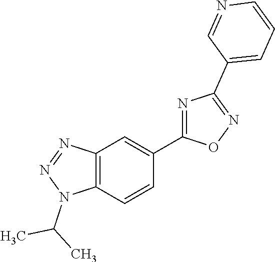 [SMILES: CC(C)n1nnc2cc(-c3nc(-c4cccnc4)no3)ccc21] | 165 | 1-(propan-2-yl)-5-[3-(pyridin-3- yl)-1,2,4-oxadiazol-5-yl]-1H- 1,2,3-benzotriazole |
| 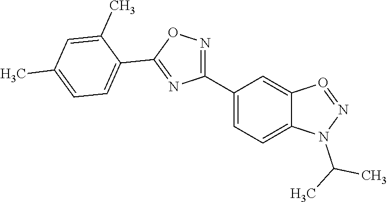 | 166 | 5-(2,4-dimethylphenyl)-3-(1- isopropyl-1H- benzo[d][1,2,3]triazol-5-yl)-1,2,4- oxadiazole |
| 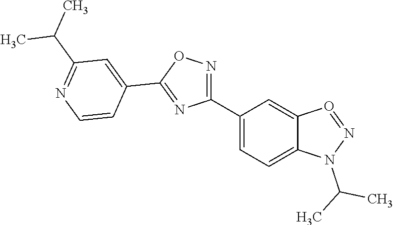 | 167 | 3-(1-isopropyl-1H- benzo[d][1,2,3]triazol-5-yl)-5-(2- isopropylpyridin-4-yl)-1,2,4- oxadiazole |
| 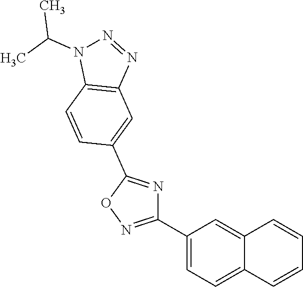 [SMILES: CC(C)n1nnc2cc(-c3nc(-c4ccc5ccccc5c4)no3)ccc21] | 168 | 2-{5-[1-(propan-2-yl)-1H-1,2,3- benzotriazol-5-yl]-1,2,4- oxadiazol-3-yl}quinoline |
| 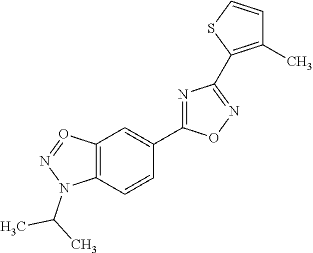 | 169 | 5-[3-(3-methylthiophen-2-yl)- 1,2,4-oxadiazol-5-yl]-1-(propan- 2-yl)-1H-1,2,3-benzotriazole |
| 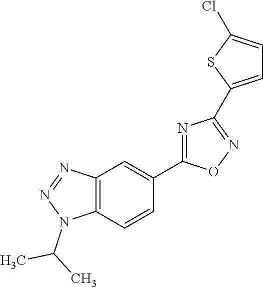 [SMILES: CC(C)n1nnc2cc(-c3nc(-c4ccc(Cl)s4)no3)ccc21] | 170 | 5-[3-(5-chlorothiophen-2-yl)- 1,2,4-oxadiazol-5-yl]-1-(propan- 2-yl)-1H-1,2,3-benzotriazole |
| 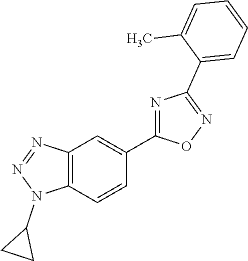 [SMILES: Cc1ccccc1-c1noc(-c2ccc3c(c2)nnn3C2CC2)n1] | 171 | 1-cyclopropyl-5-[3-(2- methylphenyl)-1,2,4-oxadiazol-5- yl]-1H-1,2,3-benzotriazole |
| 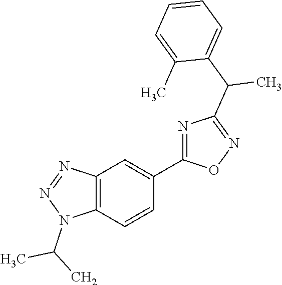 [SMILES: Cc1ccccc1C(C)c1noc(-c2ccc3c(c2)nnn3C(C)C)n1] | 172 | 5-{3-[1-(2-methylphenyl)ethyl]- 1,2,4-oxadiazol-5-yl}-1-(propan- 2-yl)-1H-1,2,3-benzotriazole |
| 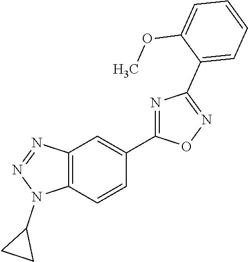 [SMILES: COc1ccccc1-c1noc(-c2ccc3c(c2)nnn3C2CC2)n1] | 173 | 1-cyclopropyl-5-[3-(2- methoxyphenyl)-1,2,4-oxadiazol- 5-yl]-1H-1,2,3-benzotriazole |
| 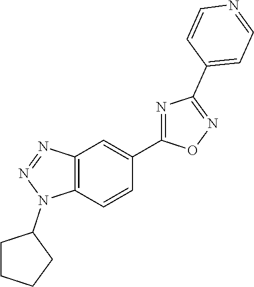 [SMILES: c1cc(-c2noc(-c3ccc4c(c3)nnn4C3CCCC3)n2)ccn1] | 174 | 1-cyclopentyl-5-[3-(pyridin-4-yl)- 1,2,4-oxadiazol-5-yl]-1H-1,2,3- benzotriazole |
| 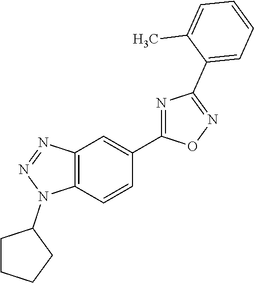 [SMILES: Cc1ccccc1-c1noc(-c2ccc3c(c2)nnn3C2CCCC2)n1] | 175 | 1-cyclopentyl-5-[3-(2- methvlphenyl)-1,2,4-oxadiazol-5- yl]-1H-1,2,3-benzotriazole |
| 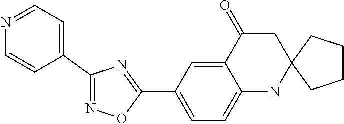 [SMILES: O=C1CC2(CCCC2)Nc2ccc(-c3nc(-c4ccncc4)no3)cc21] | 176 | 6-[3-(pyridin-4-yl)-1,2,4- oxadiazol-5-yl]-3,4- dihydrospiro[1-benzopyran-2,1′- cyclopentane]-4-one |
| 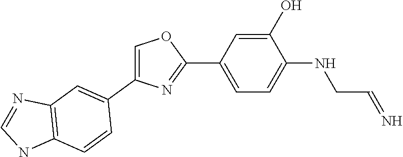 [SMILES: N=CCNc1ccc(-c2nc(-c3ccc4[nH]cnc4c3)co2)cc1O] | 177 | 5-(3-(1H-benzo[d]imidazol-5-yl)- 1,2,4-oxadiazol-5-yl)-2- (allylamino)benzonitrile |
| 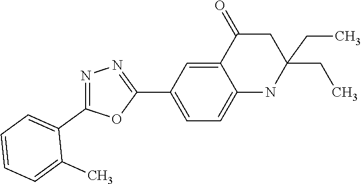 [SMILES: CCC1(CC)CC(=O)c2cc(-c3nnc(-c4ccccc4C)o3)ccc2N1] | 178 | 2,2-diethyl-6-(5-(2- methoxyphenyl)-1,3,4-thiadiazol- 2-yl)chroman-4-one |
| 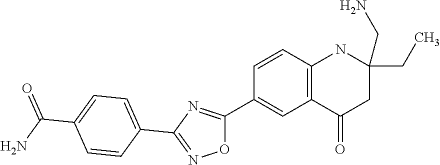 [SMILES: CCC1(CN)CC(=O)c2cc(-c3nc(-c4ccc(C(N)=O)cc4)no3)ccc2N1] | 179 | 4-(5-(2,2-diethyl-4-oxochroman- 6-yl)-1,2,4-oxadiazol-3- yl)benzamide |
| 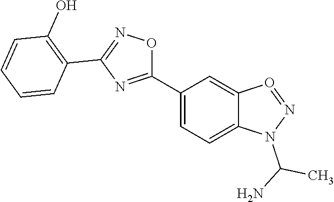 | 180 | 2-(5-(1-isopropyl-1H- benzo[d][1,2,3]triazol-5-yl)-1,2,4- oxadiazol-3-yl)phenol |
| 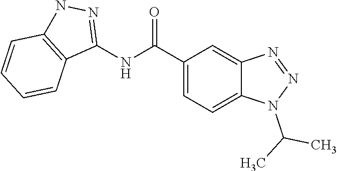 [SMILES: CC(C)n1nnc2cc(C(=O)Nc3n[nH]c4ccccc34)ccc21] | 181 | N-(benzo[d]isoxazol-3-yl)-1- isopropyl-1H- benzo[d][1,2,3]triazole-5- carboxamide |
| 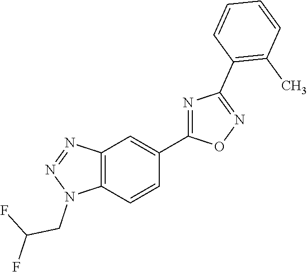 [SMILES: Cc1ccccc1-c1noc(-c2ccc3c(c2)nnn3CC(F)F)n1] | 182 | 1-(2,2-difluoroethyl)-5-[3-(2- methylphenyl)-1,2,4-oxadiazol-5- yl]-1H-1,2,3-benzotriazole |
| 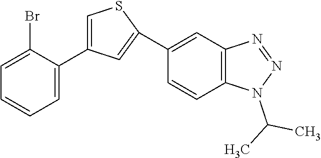 [SMILES: CC(C)n1nnc2cc(-c3cc(-c4ccccc4Br)cs3)ccc21] | 183 | 5-[4-(2-bromophenyl)-1,3-oxazol- 2-yl]-1-(propan-2-yl)-1H-1,2,3- benzotriazole |
| 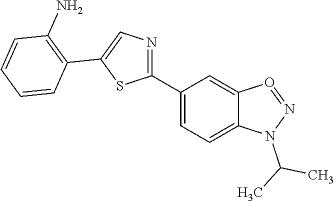 | 184 | 2-(1-isopropyl-1H- benzo[d][1,2,3]triazol-5-yl)-5-(2- methoxyphenyl)thiazole |
| 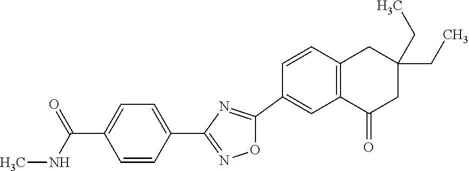 [SMILES: CCC1(CC)CC(=O)c2cc(-c3nc(-c4ccc(C(=O)NC)cc4)no3)ccc2C1] | 185 | 4-(5-(2,2-diethyl-4-oxochroman- 6-yl)-1,2,4-oxadiazol-3-yl)-N- methylbenzamide |
| 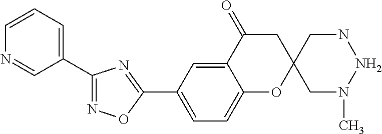 [SMILES: CN1CC2(CNN1)CC(=O)c1cc(-c3nc(-c4cccnc4)no3)ccc1O2] | 186 | 2,2-bis(methoxymethyl)-6-[3- (pyridin-3-yl)-1,2,4-oxadiazol-5- yl]-3,4-dihydro-2H-1- benzopyran-4-one |
| 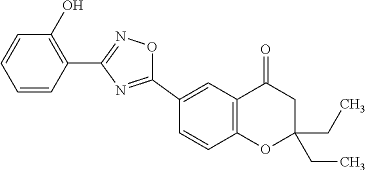 [SMILES: CCC1(CC)CC(=O)c2cc(-c3nc(-c4ccccc4O)no3)ccc2O1] | 187 | 2,2-diethyl-6-[3-(2- hydroxyphenyl)-1,2,4-oxadiazol- 5-yl]chroman-4-one |
| 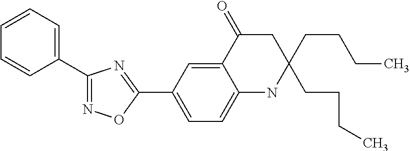 [SMILES: CCCCC1(CCCC)CC(=O)c2cc(-c3nc(-c4ccccc4)no3)ccc2N1] | 188 | 2,2-dibutyl-6-[3-(pyridin-3-yl)- 1,2,4-oxadiazol-5-yl]-3,4- dihydro-2H-1-benzopyran-4-one |
| 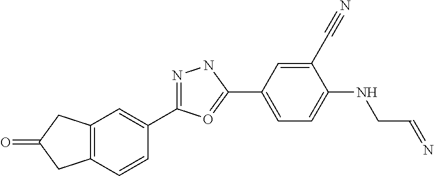 | 189 | 2-(allylamino)-5-(3-(2-oxo-2,3- dihydrobenzo[d]oxazol-6-yl)- 1,2,4-oxadiazol-5-yl)benzonitrile |
| 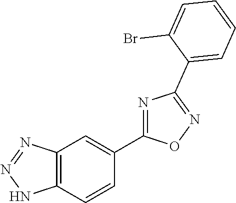 [SMILES: Brc1ccccc1-c1noc(-c2ccc3[nH]nnc3c2)n1] | 190 | 5-[3-(2-bromophenyl)-1,2,4- oxadiazol-5-yl]-1H-1,2,3- benzotriazole |
| 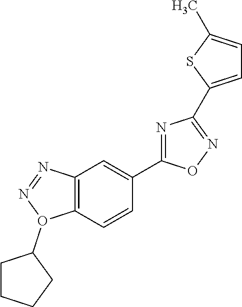 | 192 | 1-cyclopentyl-5-[3-(5- methylthiophen-2-yl)-1,2,4- oxadiazol-5-yl]-1H-1,2,3- benzotriazole |
| 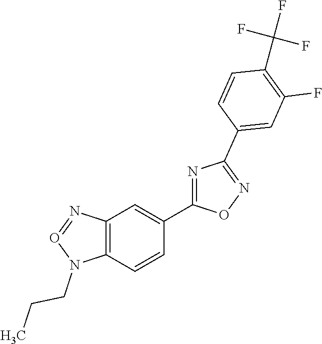 | 193 | 5-{3-[3-fluoro-4- (trifluoromethyl)phenyl]-1,2,4- oxadiazol-5-yl}-1-propyl-1H- 1,2,3-benzotriazole |
| 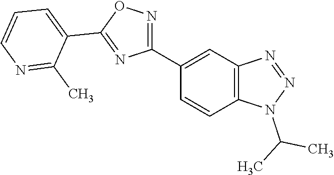 [SMILES: Cc1ncccc1-c1nc(-c2ccc3c(c2)nnn3C(C)C)no1] | 194 | 3-(1-isopropyl-1H- benzo[d][1,2,3]triazol-5-yl)-5-(2- methylpyridin-3-yl)-1,2,4- oxadiazole |
| 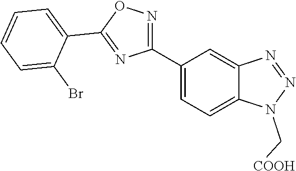 [SMILES: O=C(O)Cn1nnc2cc(-c3noc(-c4ccccc4Br)n3)ccc21] | 195 | 2-(5-(5-(2-bromophenyl)-1,2,4- oxadiazol-3-yl)-1H- benzo[d][1,2,3]triazol-1-yl)acetic acid |
| 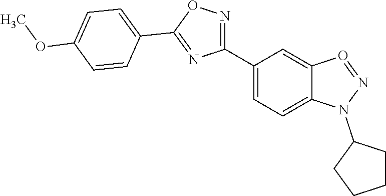 | 196 | 3-(1-cyclopentyl-1H- benzo[d][1,2,3]triazol-5-yl)-5-(4- methoxyphenyl)-1,2,4-oxadiazole |
| 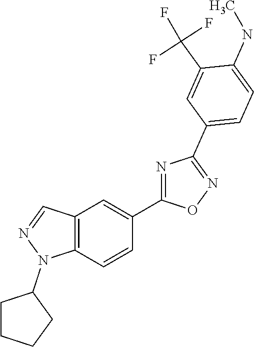 [SMILES: CNc1ccc(-c2noc(-c3ccc4c(cnn4C4CCCC4)c3)n2)cc1C(F)(F)F] | 197 | 1-cyclopentyl-5-{3-[4-methoxy-3- (trifluoromethyl)phenyl]-1,2,4- oxadiazol-5-yl}-1H-1,2,3- benzotriazole |
| 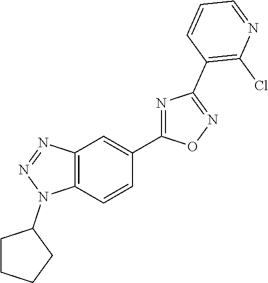 [SMILES: Clc1ncccc1-c1noc(-c2ccc3c(c2)nnn3C2CCCC2)n1] | 198 | 5-[3-(2-chloropyridin-3-yl)-1,2,4- oxadiazol-5-yl]-1-cyclopentyl- 1H-1,2,3-benzotriazole |
| 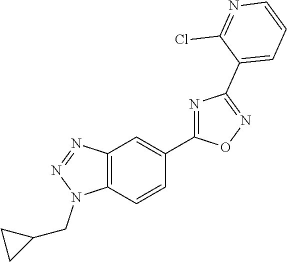 [SMILES: Clc1ncccc1-c1noc(-c2ccc3c(c2)nnn3CC2CC2)n1] | 199 | 5-[3-(2-chloropyridin-3-yl)-1,2,4- oxadiazol-5-yl]-1- (cyclopropylmethyl)-1H-1,2,3- benzotriazole |
| 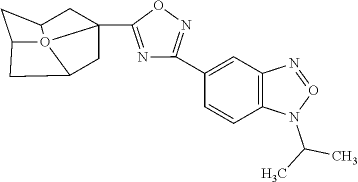 | 200 | 5-[5-(adamantan-1-yl)-1,2,4- oxadiazol-3-yl]-1-(propan-2-yl)- 1H-1,2,3-benzotriazole |
| 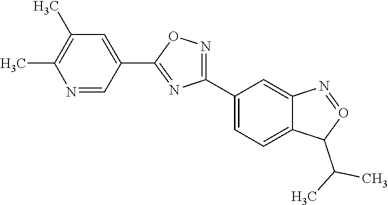 | 201 | 5-(5,6-dimethylpyridin-3-yl)-3-(1- isopropyl-1H- benzo[d][1,2,3]triazol-5-yl)-1,2,4- oxadiazole |
| 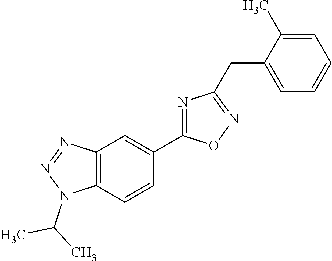 [SMILES: Cc1ccccc1Cc1noc(-c2ccc3c(c2)nnn3C(C)C)n1] | 202 | 5-{3-[(2-methylphenyl)methyl]- 1,2,4-oxadiazol-5-yl}-1-(propan- 2-yl)-1H-1,2,3-benzotriazole |
| 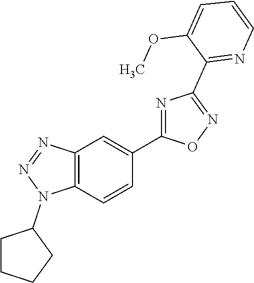 [SMILES: COc1cccnc1-c1noc(-c2ccc3c(c2)nnn3C2CCCC2)n1] | 203 | 1-cyclopentyl-5-[3-(3- methoxypyridin-2-yl)-1,2,4- oxadiazol-5-yl]-1H-1,2,3- benzotriazole |
| 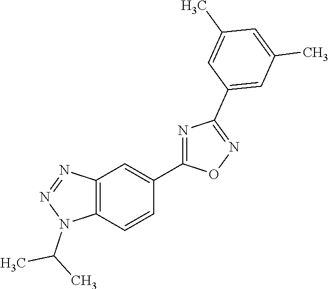 [SMILES: Cc1cc(C)cc(-c2noc(-c3ccc4c(c3)nnn4C(C)C)n2)c1] | 204 | 5-[3-(3,5-dimethylphenyl)-1,2,4- oxadiazol-5-yl]-1-(propan-2-yl)- 1H-1,2,3-benzotriazole |
| 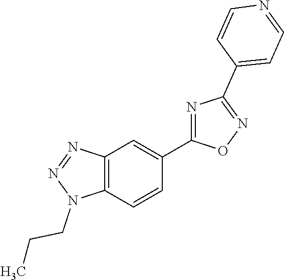 [SMILES: CCCn1nnc2cc(-c3nc(-c4ccncc4)no3)ccc21] | 205 | 1-propyl-5-[3-(pyridin-4-yl)- 1,2,4-oxadiazol-5-yl]-1H-1,2,3- benzotriazole |
| 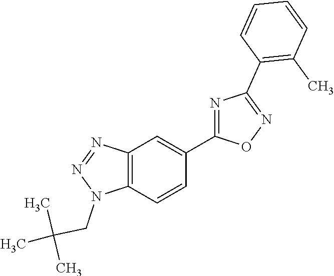 [SMILES: Cc1ccccc1-c1noc(-c2ccc3c(c2)nnn3CC(C)(C)C)n1] | 206 | 2-methyl-1-{5-[3-(4- methylpyridin-3-yl)-1,2,4- oxadiazol-5-yl]-1H-1,2,3- benzotriazol-1-yl}propan-2-ol |
| 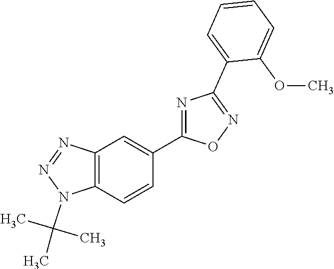 [SMILES: COc1ccccc1-c1noc(-c2ccc3c(c2)nnn3C(C)(C)C)n1] | 207 | 1-tert-butyl-5-[3-(2- methoxyphenyl)-1,2,4-oxadiazol- 5-yl]-1H-1,2,3-benzotriazole |
| 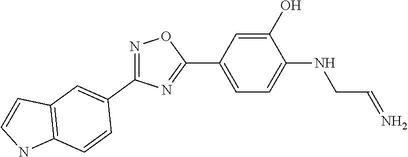 [SMILES: N=CCNc1ccc(-c2nc(-c3ccc4[nH]ccc4c3)no2)cc1O] | 208 | 5-(3-(1H-indol-5-yl)-1,2,4- oxadiazol-5-yl)-2- (allylamino)benzonitrile |
| 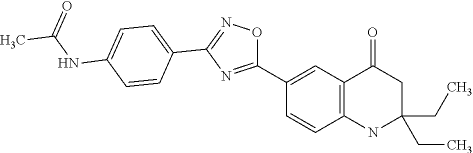 [SMILES: CCC1(CC)CC(=O)c2cc(-c3nc(-c4ccc(NC(C)=O)cc4)no3)ccc2N1] | 209 | N-(4-(5-(2,2-diethyl-4- oxochroman-6-yl)-1,2,4- oxadiazol-3-yl)phenyl)acetamide |
| 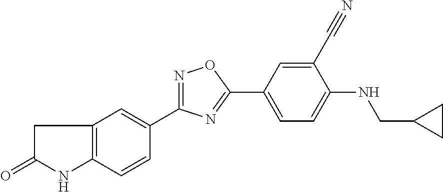 [SMILES: N#Cc1cc(-c2nc(-c3ccc4c(c3)CC(=O)N4)no2)ccc1NCC1CC1] | 210 | 2-((cyclopropylmethyl)amino)-5- (3-(2-oxoindolin-5-yl)-1,2,4- oxadiazol-5-yl)benzonitrile |
| 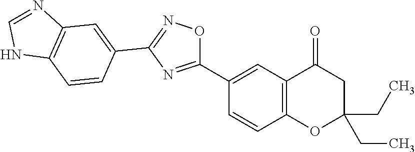 [SMILES: CCC1(CC)CC(=O)c2cc(-c3nc(-c4ccc5[nH]cnc5c4)no3)ccc2O1] | 211 | 6-(3-(1H-benzo[d]imidazol-5-yl)- 1,2,4-oxadiazol-5-yl)-2,2- diethylchroman-4-one |
| 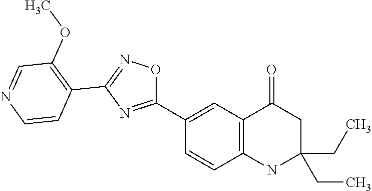 [SMILES: CCC1(CC)CC(=O)c2cc(-c3nc(-c4ccncc4OC)no3)ccc2N1] | 212 | 2,2-diethyl-6-(3-(3- methoxypyridin-4-yl)-1,2,4- oxadiazol-5-yl)chroman-4-one |
| 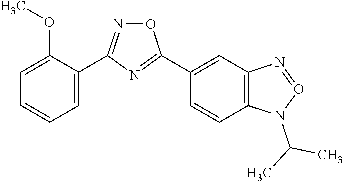 | 213 | 5-[3-(2-methoxyphenyl)-1,2,4- thiadiazol-5-yl]-1-(propan-2-yl)- 1H-1,2,3-benzotriazole |
| 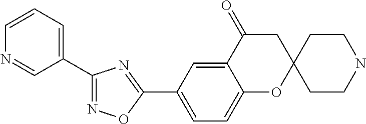 [SMILES: O=C1CC2(CCNCC2)Oc2ccc(-c3nc(-c4cccnc4)no3)cc21] | 214 | 6-[3-(pyridin-3-yl)-1,2,4- oxadiazol-5-yl]-3,4- dihydrospiro[1-benzopyran-2,4′- oxane]-4-one |
| 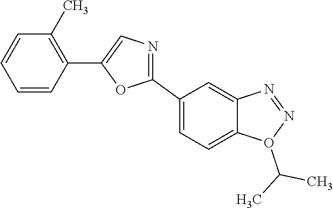 | 215 | 2-(1-isopropyl-1H- benzo[d][1,2,3]triazol-5-yl)-5-(2- (trifluoromethyl)phenyl)thiazole |
| 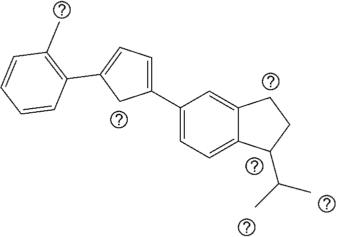 [SMILES: Cc1ccccc1C1=CC=C(c2ccc3c(c2)CCC3C(C)C)C1] | 216 | 2-(1-isopropylbenzotriazol-5-yl)- 5-(o-tolyl)thiazole |
| 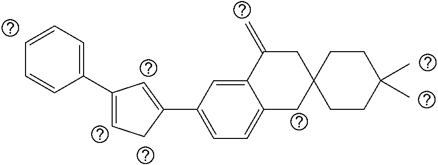 [SMILES: C=C1CC2(CCC(C)(C)CC2)Cc2ccc(C3=CC(c4ccccc4)=CC3)cc21] | 217 | 4′,4′-difluoro-6-[3-(pyridin-4-yl)- 1,2,4-oxadiazol-5-yl]-3,4- dihydrospiro[1-benzopyran-2,1′- cyclohexane]-4-one |
| 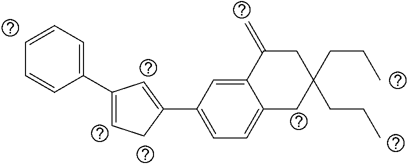 [SMILES: C=C1CC(CCC)(CCC)Cc2ccc(C3=CC(c4ccccc4)=CC3)cc21] | 218 | 2,2-dipropyl-6-[3-(pyridin-3-yl)- 1,2,4-oxadiazol-5-yl]-3,4- dihydro-2H-1-benzopyran-4-one |
| 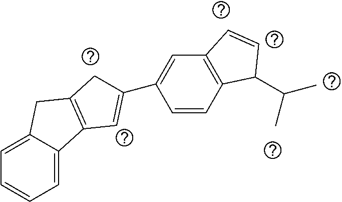 [SMILES: CC(C)C1C=Cc2cc(C3=CC4=C(C3)Cc3ccccc34)ccc21] | 219 | 5-{8H-indeno[1,2-d][1,3]oxazol- 2-yl}-1-(propan-2-yl)-1H-1,2,3- benzotriazole |
| 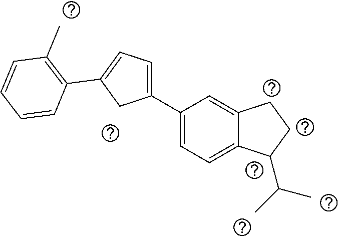 [SMILES: Cc1ccccc1C1=CC=C(c2ccc3c(c2)CCC3C(C)C)C1] | 220 | 5-(2-bromophenyl)-2-(1- isopropylbenzotriazol-5- yl)thiazole |
| 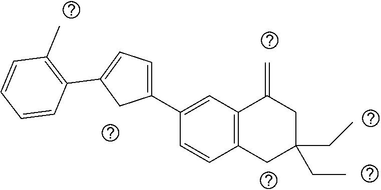 [SMILES: C=C1CC(CC)(CC)Cc2ccc(C3=CC=C(c4ccccc4C)C3)cc21] | 221 | 6-(3-(2-chlorophenyl)-1,2,4- oxadiazol-5-yl)-2,2- diethylchroman-4-one |
| 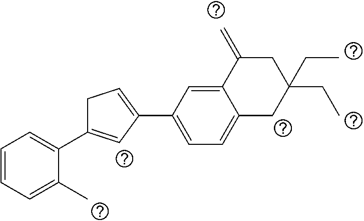 [SMILES: C=C1CC(CC)(CC)Cc2ccc(C3=CCC(c4ccccc4C)=C3)cc21] | 222 | 2,2-diethyl-6-(5-(2- methoxyphenyl)-1,2,4-thiadiazol- 3-yl)chroman-4-one |
| 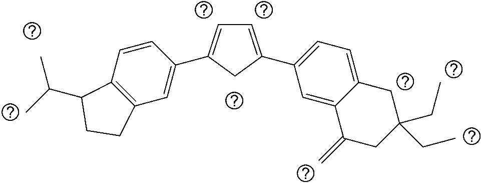 [SMILES: C=C1CC(CC)(CC)Cc2ccc(C3=CC=C(c4ccc5c(c4)CCC5C(C)C)C3)cc21] | 223 | 2,2-diethyl-6-(5-(1-isopropyl-1H- benzo[d][1,2,3]triazol-5-yl)-1,3,4- oxadiazol-2-yl)chroman-4-one |
| 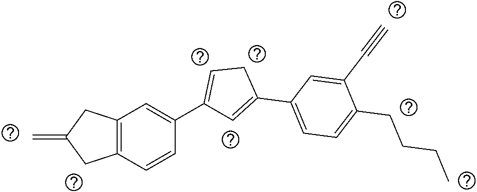 [SMILES: C#Cc1cc(C2=CC(c3ccc4c(c3)CC(=C)C4)=CC2)ccc1CCCC] | 224 | 2-(allylamino)-5-(3-(2- oxoindolin-5-yl)-1,2,4-oxadiazol- 5-yl)benzonitrile |
| 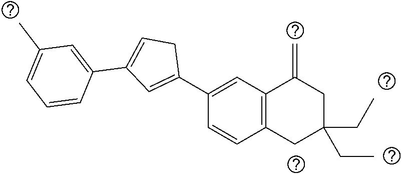 [SMILES: C=C1CC(CC)(CC)Cc2ccc(C3=CC(c4cccc(C)c4)=CC3)cc21] | 225 | 2,2-diethyl-6-(3-(m-tolyl)-1,2,4- oxadiazol-5-yl)chroman-4-one |
| 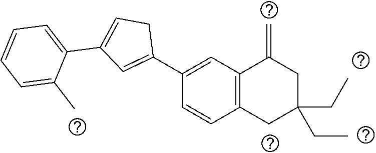 [SMILES: C=C1CC(CC)(CC)Cc2ccc(C3=CC(c4ccccc4C)=CC3)cc21] | 226 | 2,2-diethyl-6-(3-(3-fluoropyridin- 4-yl)-1,2,4-oxadiazol-5- yl)chroman-4-one |
| 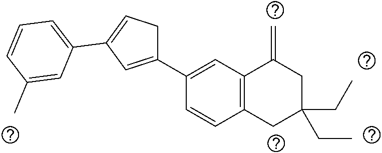 [SMILES: C=C1CC(CC)(CC)Cc2ccc(C3=CC(c4cccc(C)c4)=CC3)cc21] | 227 | 2,2-diethyl-6-[3-(3- hydroxyphenyl)-1,2,4-oxadiazol- 5-yl]chroman-4-one |
| 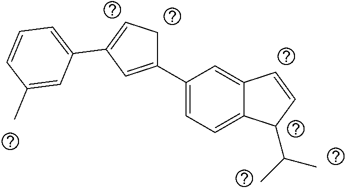 [SMILES: Cc1cccc(C2=CCC(c3ccc4c(c3)C=CC4C(C)C)=C2)c1] | 228 | 5-(1-isopropyl-1H- benzo[d][1,2,3]triazol-5-yl)-3-(3- methoxyphenyl)-1,2,4-oxadiazole |
| 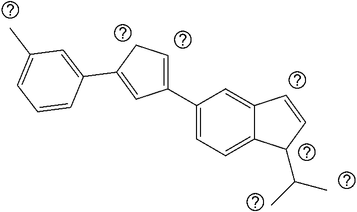 [SMILES: Cc1cccc(C2=CC(c3ccc4c(c3)C=CC4C(C)C)=CC2)c1] | 229 | 3-(1-isopropyl-1H- benzo[d][1,2,3]triazol-5-yl)-5-(6- methylpyrazin-2-yl)-1,2,4- oxadiazole |
| 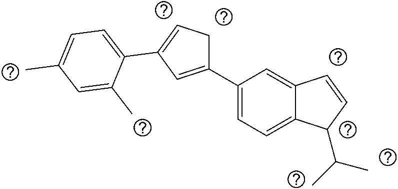 [SMILES: Cc1ccc(C2=CCC(c3ccc4c(c3)C=CC4C(C)C)=C2)c(C)c1] | 230 | 3-(3,4-dimethoxyphenyl)-5-(1- isopropyl-1H- benzo[d][1,2,3]triazol-5-yl)-1,2,4- oxadiazole |
| 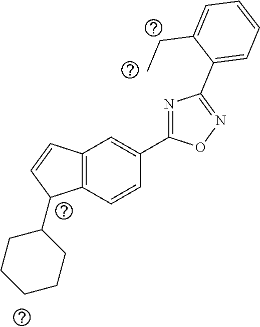 [SMILES: CCc1ccccc1-c1noc(-c2ccc3c(c2)C=CC3C2CCCCC2)n1] | 231 | 5-[3-(2-methoxyphenyl)-1,2,4- oxadiazol-5-yl]-1-(oxan-4-yl)- 1H-1,2,3-benzotriazole |
| 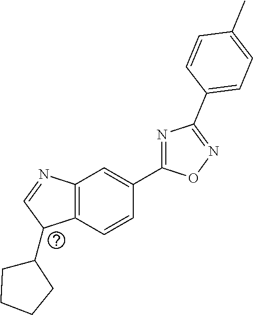 [SMILES: Cc1ccc(-c2noc(-c3ccc4c(c3)N=CC4C3CCCC3)n2)cc1] | 232 | 1-cyclopentyl-5-[3-(4- fluorophenyl)-1,2,4-oxadiazol-5- yl]-1H-1,2,3-benzotriazole |
| 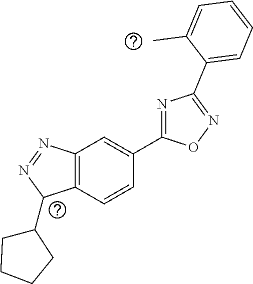 [SMILES: Cc1ccccc1-c1noc(-c2ccc3c(c2)N=NC3C2CCCC2)n1] | 233 | 5-[3-(2-bromophenyl)-1,2,4- oxadiazol-5-yl]-1-(oxan-4-yl)- 1H-1,2,3-benzotriazole |
| 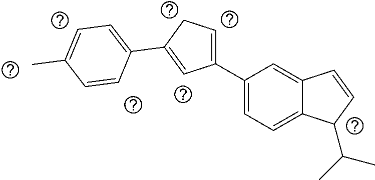 [SMILES: Cc1ccc(C2=CC(c3ccc4c(c3)C=CC4C(C)C)=CC2)cc1] | 234 | 3-(1-isopropyl-1H- benzo[d][1,2,3]triazol-5-yl)-5-(5- methylpyrazin-2-yl)-1,2,4- oxadiazole |
| 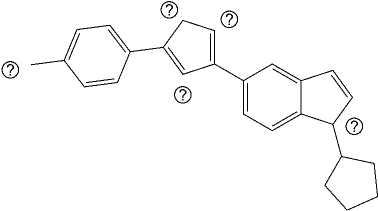 [SMILES: Cc1ccc(C2=CC(c3ccc4c(c3)C=CC4C3CCCC3)=CC2)cc1] | 235 | 3-(1-cyclopentyl-1H- benzo[d][1,2,3]triazol-5-yl)-5-(p- tolyl)-1,2,4-oxadiazole |
| 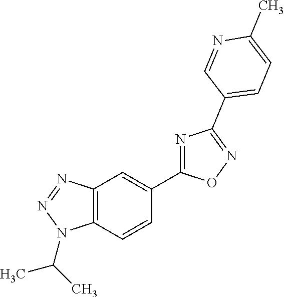 [SMILES: Cc1ccc(-c2noc(-c3ccc4c(c3)nnn4C(C)C)n2)cn1] | 236 | 5-[3-(6-methylpyridin-3-yl)-1,2,4- oxadiazol-5-yl]-1-(propan-2-yl)- 1H-1,2,3-benzotriazole |
| 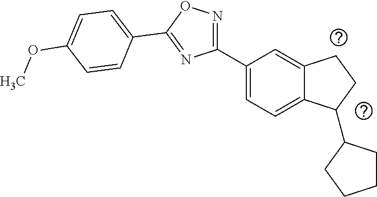 [SMILES: COc1ccc(-c2nc(-c3ccc4c(c3)CCC4C3CCCC3)no2)cc1] | 237 | 3-(1-cyclopentyl-1H- benzo[d][1,2,3]triazol-5-yl)-5-(4- (methylthio)phenyl)-1,2,4- oxadiazole |
| 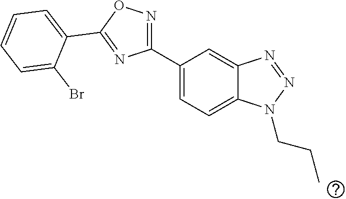 [SMILES: CCCn1nnc2cc(-c3noc(-c4ccccc4Br)n3)ccc21] | 238 | 2-(5-(5-(2-bromophenyl)-1,2,4- oxadiazol-3-yl)-1H- benzo[d][1,2,3]triazol-1- yl)ethanol |
| 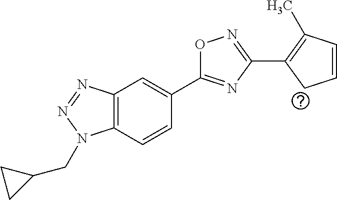 [SMILES: CC1=C(c2noc(-c3ccc4c(c3)nnn4CC3CC3)n2)CC=C1] | 239 | 1-(cyclopropylmethyl)-5-[3-(3- methylthiophen-2-yl)-1,2,4- oxadiazol-5-yl]-1H-1,2,3- benzotriazole |
| 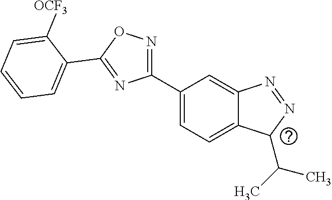 [SMILES: CC(C)C1N=Nc2cc(-c3noc(-c4ccccc4OC(F)(F)F)n3)ccc21] | 240 | 3-(1-isopropyl-1H- benzo[d][1,2,3]triazol-5-yl)-5-(2- (trifluoromethoxy)phenyl)-1,2,4- oxadiazole |
| 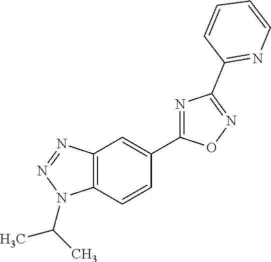 [SMILES: CC(C)n1nnc2cc(-c3nc(-c4ccccn4)no3)ccc21] | 241 | 1-(propan-2-yl)-5-[3-(pyridin-2- yl)-1,2,4-oxadiazol-5-yl]-1H- 1,2,3-benzotriazole |
| 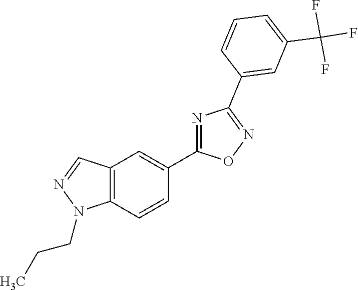 [SMILES: CCCn1ncc2cc(-c3nc(-c4cccc(C(F)(F)F)c4)no3)ccc21] | 242 | 1-propyl-5-{3-[3- (trifluoromethyl)phenyl]-1,2,4- oxadiazol-5-yl}-1H-1,2,3- benzotriazole |
| 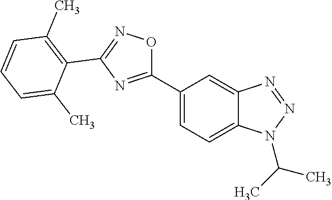 [SMILES: Cc1cccc(C)c1-c1noc(-c2ccc3c(c2)nnn3C(C)C)n1] | 243 | 3-(2,6-dimethylphenyl)-5-(1- isopropyl-1H- benzo[d][1,2,3]triazol-5-yl)-1,2,4- oxadiazole |
| 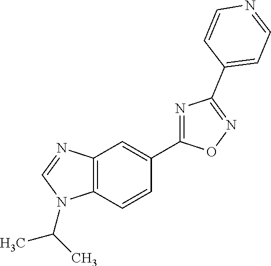 [SMILES: CC(C)n1cnc2cc(-c3nc(-c4ccncc4)no3)ccc21] | 244 | 1-(propan-2-yl)-5-[3-(pyridin-4- yl)-1,2,4-oxadiazol-5-yl]-1H- 1,2,3-benzotriazole |
| 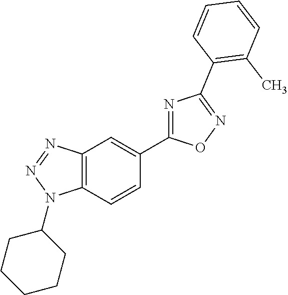 [SMILES: Cc1ccccc1-c1noc(-c2ccc3c(c2)nnn3C2CCCCC2)n1] | 245 | 1-cyclohexyl-5-[3-(2- methylphenyl)-1,2,4-oxadiazol-5- yl]-1H-1,2,3-benzotriazole |
| 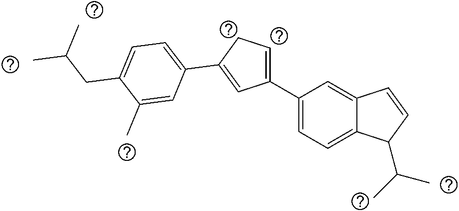 [SMILES: Cc1cc(C2=CC(c3ccc4c(c3)C=CC4C(C)C)=CC2)ccc1CC(C)C] | 246 | 5-(4-isopropoxy-3- (trifluoromethyl)phenyl)-3-(1- isopropyl-1H- benzo[d][1,2,3]triazol-5-yl)-1,2,4- oxadiazole |
| 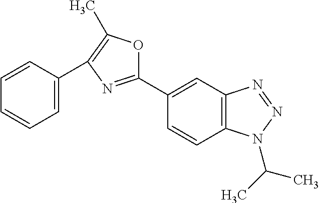 [SMILES: Cc1oc(-c2ccc3c(c2)nnn3C(C)C)nc1-c1ccccc1] | 247 | 5-(5-methyl-4-phenyl-1,3-oxazol- 2-yl)-1-(propan-2-yl)-1H-1,2,3- benzotriazole |
| 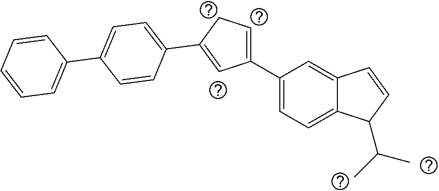 [SMILES: CC(C)C1C=Cc2cc(C3=CCC(c4ccc(-c5ccccc5)cc4)=C3)ccc21] | 248 | 5-([1,1′-biphenyl]-4-yl)-3-(1- isopropyl-1H- benzo[d][1,2,3]triazol-5-yl)-1,2,4- oxadiazole |
| 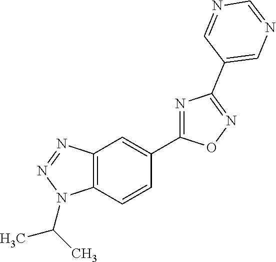 [SMILES: CC(C)n1nnc2cc(-c3nc(-c4cncnc4)no3)ccc21] | 249 | 1-(propan-2-yl)-5-[3-(pyrimidin- 5-yl)-1,2,4-oxadiazol-5-yl]-1H- 1,2,3-benzotriazole |
| 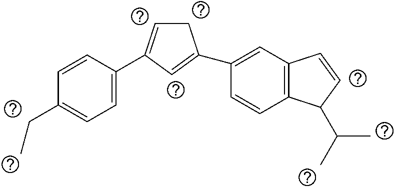 [SMILES: CCc1ccc(C2=CCC(c3ccc4c(c3)C=CC4C(C)C)=C2)cc1] | 250 | 5-[4-(4-methoxyphenyl)-1,3- oxazol-2-yl]-1-(propan-2-yl)-1H- 1,2,3-benzotriazole |
| 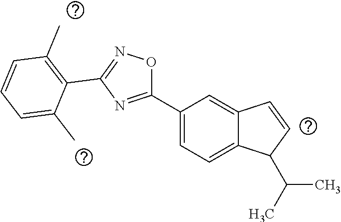 [SMILES: Cc1cccc(C)c1-c1noc(-c2ccc3c(c2)C=CC3C(C)C)n1] | 251 | 3-(2,6-dimethoxyphenyl)-5-(1- isopropylbenzotriazol-5-yl)-1,2,4- oxadiazole |
| 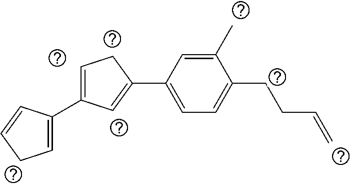 [SMILES: C=CCCc1ccc(C2=CC(C3=CCC=C3)=CC2)cc1C] | 252 | 5-(3-(1H-pyrazol-4-yl)-1,2,4- oxadiazol-5-yl)-2- (allylamino)benzonitrile |
| 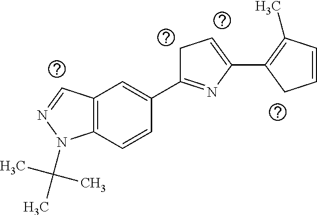 [SMILES: CC1=C(C2=CCC(c3ccc4c(cnn4C(C)(C)C)c3)=N2)CC=C1] | 253 | 1-tert-butyl-5-[3-(3- methylthiophen-2-yl)-1,2,4- oxadiazol-5-yl]-1H-1,2,3- benzotriazole |
| 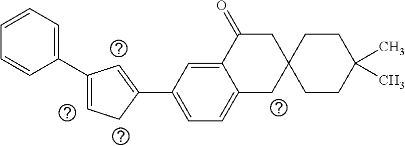 [SMILES: CC1(C)CCC2(CC1)CC(=O)c1cc(C3=CC(c4ccccc4)=CC3)ccc1C2] | 254 | 4′,4′-dimethyl-6-[3-(pyridin-4-yl)- 1,2,4-oxadiazol-5-yl]-3,4- dihydrospiro[1-benzopyran-2,1′- cyclohexane]-4-one |
| 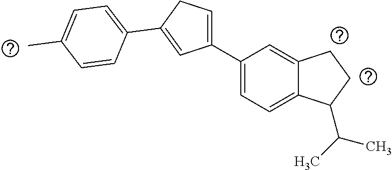 [SMILES: Cc1ccc(C2=CC(c3ccc4c(c3)CCC4C(C)C)=CC2)cc1] | 255 | 5-(4-fluorophenyl)-3-(1- isopropylbenzotriazol-5-yl)-1,2,4- oxadiazole |
| 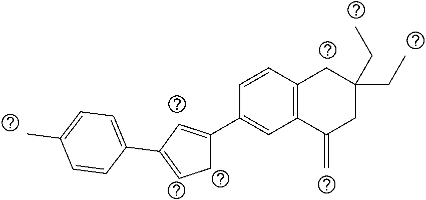 [SMILES: C=C1CC(CC)(CC)Cc2ccc(C3=CC(c4ccc(C)cc4)=CC3)cc21] | 256 | 6-(3-(4-aminophenyl)-1,2,4- oxadiazol-5-yl)-2,2- diethylchroman-4-one |
| 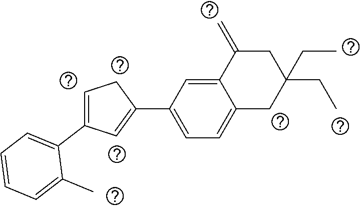 [SMILES: C=C1CC(CC)(CC)Cc2ccc(C3=CC(c4ccccc4C)=CC3)cc21] | 257 | 2,2-diethyl-6-(3-(2- methoxyphenyl)-1,2,4-thiadiazol- 5-yl)chroman-4-one |
| 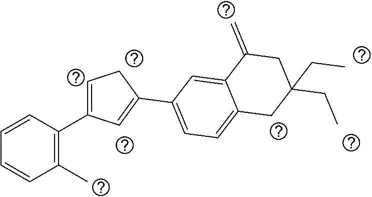 [SMILES: C=C1CC(CC)(CC)Cc2ccc(C3=CC(c4ccccc4C)=CC3)cc21] | 258 | 2,2-diethyl-6-(3-(2- hydroxypyridin-3-yl)-1,2,4- oxadiazol-5-yl)chroman-4-one |
| 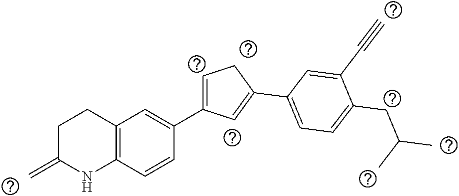 [SMILES: C#Cc1cc(C2=CC(c3ccc4c(c3)CCC(=C)N4)=CC2)ccc1CC(C)C] | 259 | 2-(isopropylamino)-5-(3-(2-oxo- 1,2,3,4-tetrahydroquinolin-6-yl)- 1,2,4-oxadiazol-5-yl)benzonitrile |
| 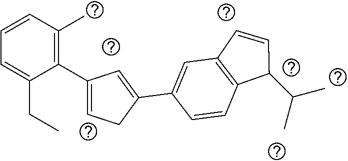 [SMILES: CCc1cccc(C)c1C1=CCC(c2ccc3c(c2)C=CC3C(C)C)=C1] | 260 | 3-(2-fluoro-6-methoxy-phenyl)-5- (1-isopropylbenzotriazol-5-yl)- 1,2,4-oxadiazole |
| 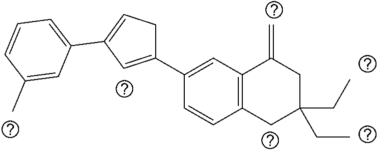 [SMILES: C=C1CC(CC)(CC)Cc2ccc(C3=CC(c4cccc(C)c4)=CC3)cc21] | 261 | 6-[3-(3-chlorophenyl)-1,2,4- oxadiazol-5-yl]-2,2-diethyl- chroman-4-one |
| 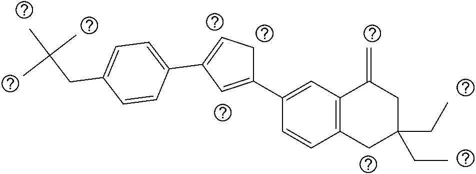 [SMILES: C=C1CC(CC)(CC)Cc2ccc(C3=CC(c4ccc(CC(C)(C)C)cc4)=CC3)cc21] | 262 | N-(4-(5-(2,2-diethyl-4- oxochroman-6-yl)-1,2,4- oxadiazol-3- yl)phenyl)methanesulfonamide |
| 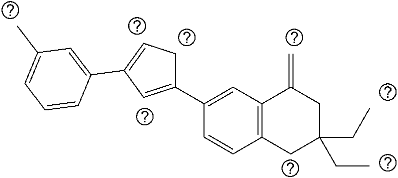 [SMILES: C=C1CC(CC)(CC)Cc2ccc(C3=CC(c4cccc(C)c4)=CC3)cc21] | 263 | 2,2-diethyl-6-[3-(2-hydroxy-4- pyridyl)-1,2,4-oxadiazol-5- yl]chroman-4-one |
| 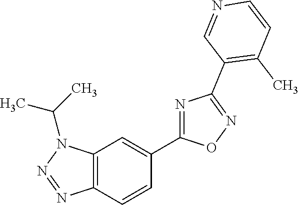 [SMILES: Cc1ccncc1-c1noc(-c2ccc3nnn(C(C)C)c3c2)n1] | 264 | 6-[3-(4-methylpyridin-3-yl)-1,2,4- oxadiazol-5-yl]-1-(propan-2-yl)- 1H-1,2,3-benzotriazole |
| 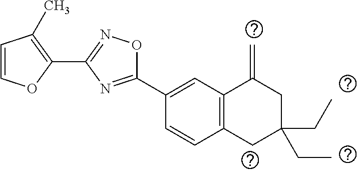 [SMILES: C=C1CC(CC)(CC)Cc2ccc(-c3nc(-c4occc4C)no3)cc21] | 265 | 2,2-diethyl-6-(3-(3- methylthiophen-2-yl)-1,2,4- oxadiazol-5-yl)chroman-4-one |
| 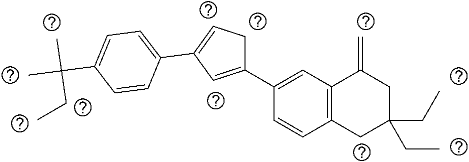 [SMILES: C=C1CC(CC)(CC)Cc2ccc(C3=CC(c4ccc(C(C)(C)CC)cc4)=CC3)cc21] | 266 | 4-(5-(2,2-diethyl-4-oxochroman- 6-yl)-1,2,4-oxadiazol-3-yl)-N- methylbenzenesulfonamide |
| 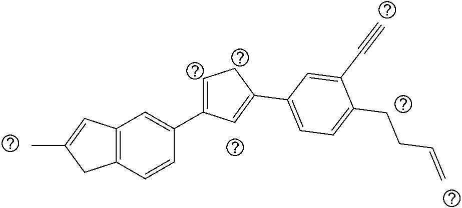 [SMILES: C#Cc1cc(C2=CC(c3ccc4c(c3)C=C(C)C4)=CC2)ccc1CCC=C] | 267 | 2-(allylamino)-5-(3-(2- aminobenzo[d]thiazol-5-yl)-1,2,4- oxadiazol-5-yl)benzonitrile |
| 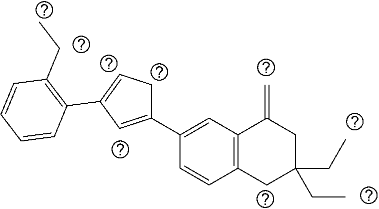 [SMILES: C=C1CC(CC)(CC)Cc2ccc(C3=CC(c4ccccc4CC)=CC3)cc21] | 268 | 2,2-diethyl-6-[3-(2- methoxyphenyl)-1,2,4-oxadiazol- 5-yl]chroman-4-one |
| 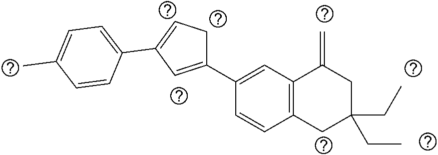 [SMILES: C=C1CC(CC)(CC)Cc2ccc(C3=CC(c4ccc(C)cc4)=CC3)cc21] | 269 | 2,2-diethyl-6-[3-(p-tolyl)-1,2,4- oxadiazol-5-yl]chroman-4-one |
| 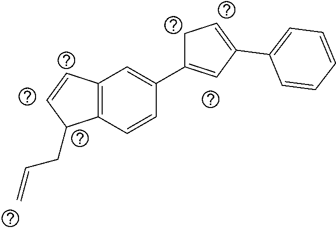 [SMILES: C=CCC1C=Cc2cc(C3=CC(c4ccccc4)=CC3)ccc21] | 270 | 5-(3-phenyl-1,2,4-oxadiazol-5- yl)-1-(prop-2-en-1-yl)-1H-1,2,3- benzotriazole |
| 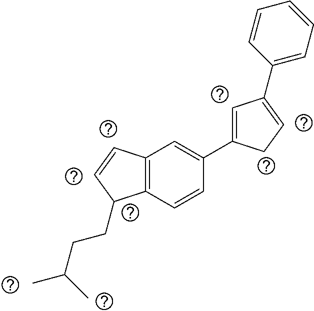 [SMILES: CC(C)CCC1C=Cc2cc(C3=CC(c4ccccc4)=CC3)ccc21] | 271 | 1-(3-methylbutyl)-5-(3-phenyl- 1,2,4-oxadiazol-5-yl)-1H-1,2,3- benzotriazole |
| 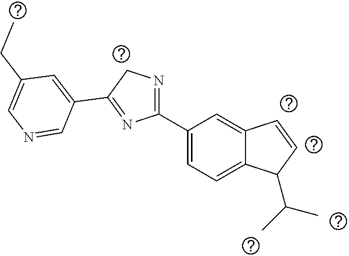 [SMILES: CCc1cncc(C2=NC(c3ccc4c(c3)C=CC4C(C)C)=NC2)c1] | 272 | 3-(1-isopropyl-1H- benzo[d][1,2,3]triazol-5-yl)-5-(5- methoxypyridin-3-yl)-1,2,4- oxadiazole |
| 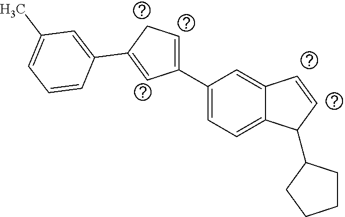 [SMILES: Cc1cccc(C2=CC(c3ccc4c(c3)C=CC4C3CCCC3)=CC2)c1] | 273 | 3-(1-cyclopentyl-1H- benzo[d][1,2,3]triazol-5-yl)-5-(m- tolyl)-1,2,4-oxadiazole |
| 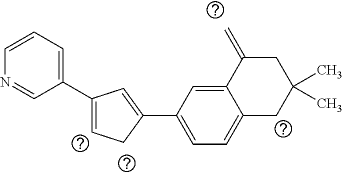 [SMILES: C=C1CC(C)(C)Cc2ccc(C3=CC(c4cccnc4)=CC3)cc21] | 274 | 2,2-dimethyl-6-[3-(pyridin-3-yl)- 1,2,4-oxadiazol-5-yl]-3,4- dihydro-2H-1-benzopyran-4-one |
| 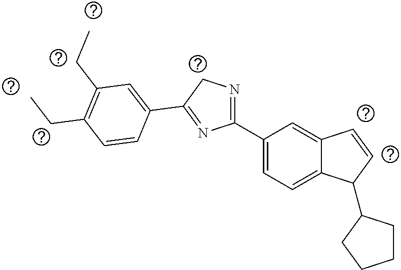 [SMILES: CCc1ccc(C2=NC(c3ccc4c(c3)C=CC4C3CCCC3)=NC2)cc1CC] | 275 | 3-(1-cyclopentyl-1H- benzo[d][1,2,3]triazol-5-yl)-5- (3,4-dimethoxyphenyl)-1,2,4- oxadiazole |
| 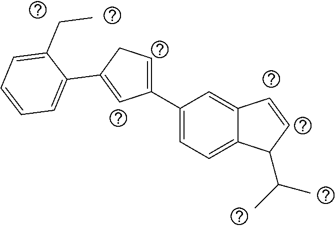 [SMILES: CCc1ccccc1C1=CC(c2ccc3c(c2)C=CC3C(C)C)=CC1] | 276 | 3-(1-isopropyl-1H- benzo[d][1,2,3]triazol-5-yl)-5-(2- (methylthio)phenyl)-1,2,4- oxadiazole |
| 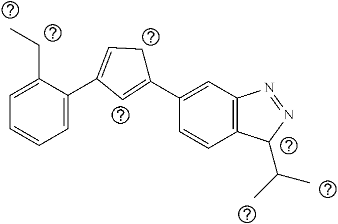 [SMILES: CCc1ccccc1C1=CCC(c2ccc3c(c2)N=NC3C(C)C)=C1] | 277 | 5-[4-(2-methoxyphenyl)-1,3- oxazol-2-yl]-1-(propan-2-yl)-1H- 1,2,3-benzotriazole |
| 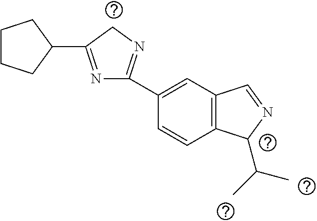 [SMILES: CC(C)C1N=Cc2cc(C3=NCC(C4CCCC4)=N3)ccc21] | 278 | 5-(5-cyclopentyl-1,2,4-oxadiazol- 3-yl)-1-(propan-2-yl)-1H-1,2,3- benzotriazole5-(5-cyclopentyl- 1,2,4-oxadiazol-3-yl)-1-(propan- 2-yl)-1H-1,2,3-benzotriazole |
| 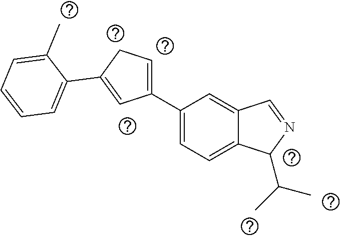 [SMILES: Cc1ccccc1C1=CC(c2ccc3c(c2)C=NC3C(C)C)=CC1] | 279 | 2-(3-(1-isopropyl-1H- benzo[d][1,2,3]triazol-5-yl)-1,2,4- oxadiazol-5-yl)benzonitrile |
| 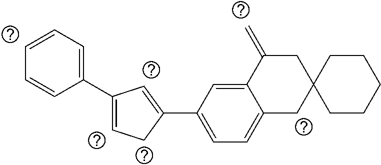 [SMILES: C=C1CC2(CCCCC2)Cc2ccc(C3=CC(c4ccccc4)=CC3)cc21] | 280 | 6-[3-(pyridin-4-yl)-1,2,4- oxadiazol-5-yl]-3,4- dihydrospiro[1-benzopyran-2,1′- cyclohexane]-4-one |
| 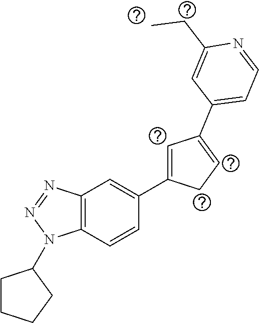 [SMILES: CCc1cc(C2=CCC(c3ccc4c(c3)nnn4C3CCCC3)=C2)ccn1] | 281 | 1-cyclopentyl-5-[3-(2- methoxypyridin-4-yl)-1,2,4- oxadiazol-5-yl]-1H-1,2,3- benzotriazole |
| 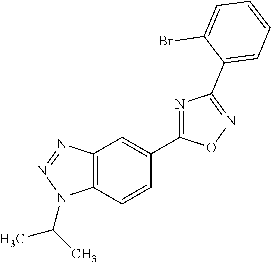 [SMILES: CC(C)n1nnc2cc(-c3nc(-c4ccccc4Br)no3)ccc21] | 282 | 5-[3-(2-bromophenyl)-1,2,4- oxadiazol-5-yl]-1-(propan-2-yl)- 1H-1,2,3-benzotriazole |
| 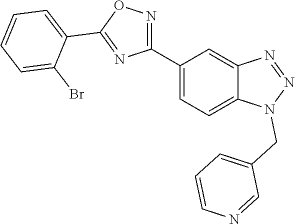 [SMILES: Brc1ccccc1-c1nc(-c2ccc3c(c2)nnn3Cc2cccnc2)no1] | 283 | 5-(2-bromophenyl)-3-(1-(pyridin- 3-ylmethyl)-1H- benzo[d][1,2,3]triazol-5-yl)-1,2,4- oxadiazole |
| 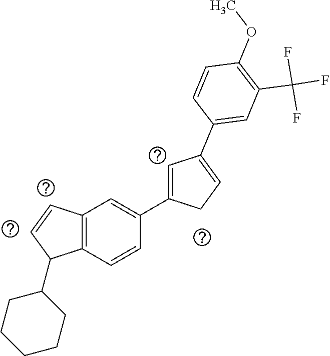 [SMILES: COc1ccc(C2=CCC(c3ccc4c(c3)C=CC4C3CCCCC3)=C2)cc1C(F)(F)F] | 284 | 1-cyclohexyl-5-{3-[4-methoxy-3- (trifluoromethyl)phenyl]-1,2,4- oxadiazol-5-yl}-1H-1,2,3- benzotriazole |
| 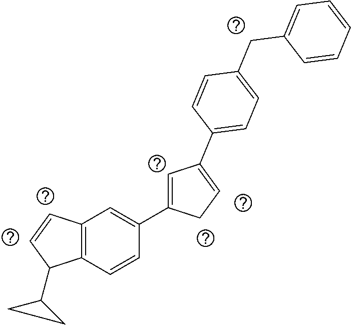 [SMILES: C1=CC(C2CC2)c2ccc(C3=CC(c4ccc(Cc5ccccc5)cc4)=CC3)cc21] | 285 | 1-cyclopropyl-5-[3-(4- phenoxyphenyl)-1,2,4-oxadiazol- 5-yl]-1H-1,2,3-benzotriazole |
| 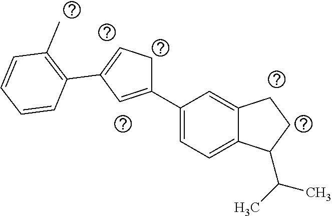 [SMILES: Cc1ccccc1C1=CCC(c2ccc3c(c2)CCC3C(C)C)=C1] | 286 | 5-(1-isopropyl-1H- benzo[d][1,2,3]triazol-5-yl)-3-(2- methoxyphenyl)-1,2,4-oxadiazole |
| 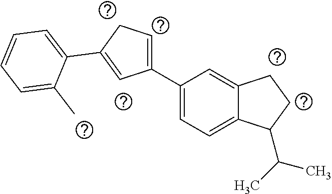 [SMILES: Cc1ccccc1C1=CC(c2ccc3c(c2)CCC3C(C)C)=CC1] | 287 | 3-(1-isopropyl-1H- benzo[d][1,2,3]triazol-5-yl)-5-(2- methoxyphenyl)-1,2,4-oxadiazole |
| 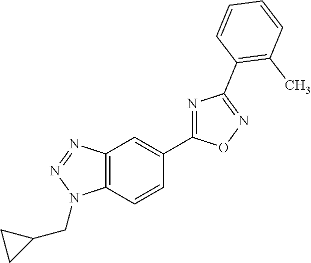 [SMILES: Cc1ccccc1-c1noc(-c2ccc3c(c2)nnn3CC2CC2)n1] | 288 | 1-(cyclopropylmethyl)-5-[3-(2- methylphenyl)-1,2,4-oxadiazol-5- yl]-1H-1,2,3-benzotriazole |
| 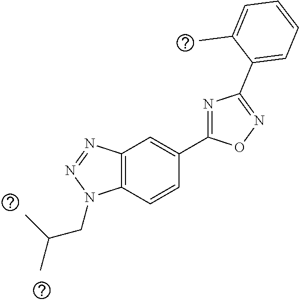 [SMILES: Cc1ccccc1-c1noc(-c2ccc3c(c2)nnn3CC(C)C)n1] | 289 | 5-[3-(2-chlorophenyl)-1,2,4- oxadiazol-5-yl]-1-(2- methylpropyl)-1H-1,2,3- benzotriazole |
| 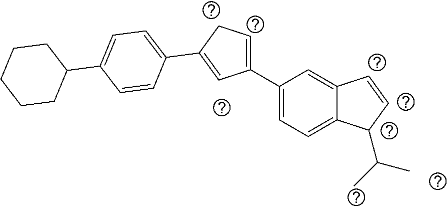 [SMILES: CC(C)C1C=Cc2cc(C3=CCC(c4ccc(C5CCCCC5)cc4)=C3)ccc21] | 290 | 5-(4-cyclohexylphenyl)-3-(1- isopropyl-1H- benzo[d][1,2,3]triazol-5-yl)-1,2,4- oxadiazole |
| 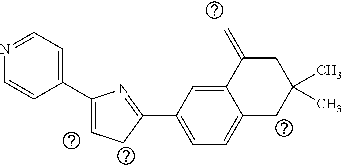 [SMILES: C=C1CC(C)(C)Cc2ccc(C3=NC(c4ccncc4)=CC3)cc21] | 291 | 2,2-dimethyl-6-[3-(pyridin-4-yl)- 1,2,4-oxadiazol-5-yl]-3,4- dihydro-2H-1-benzopyran-4-one |
| 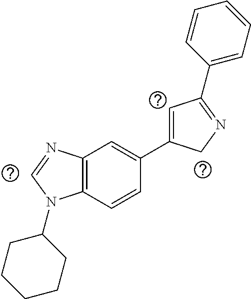 [SMILES: C1=C(c2ccc3c(c2)ncn3C2CCCCC2)CN=C1c1ccccc1] | 292 | 1-cyclohexyl-5-(3-phenyl-1,2,4- oxadiazol-5-yl)-1H-1,2,3- benzotriazole |
| 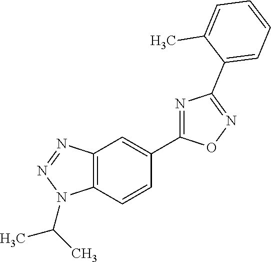 [SMILES: Cc1ccccc1-c1noc(-c2ccc3c(c2)nnn3C(C)C)n1] | 293 | 5-[3-(2-methylphenyl)-1,2,4- oxadiazol-5-yl]-1-(propan-2-yl)- 1H-1,2,3-benzotriazole |
| 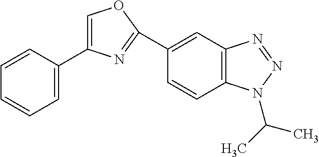 [SMILES: CC(C)n1nnc2cc(-c3nc(-c4ccccc4)co3)ccc21] | 294 | 5-(4-phenyl-1,3-oxazol-2-yl)-1- (propan-2-yl)-1H-1,2,3- benzotriazole |
| 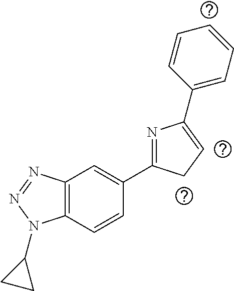 [SMILES: C1=C(c2ccccc2)N=C(c2ccc3c(c2)nnn3C2CC2)C1] | 295 | 1-cyclopropyl-5-[3-(pyridin-4-yl)- 1,2,4-oxadiazol-5-yl]-1H-1,2,3- benzotriazole |
| 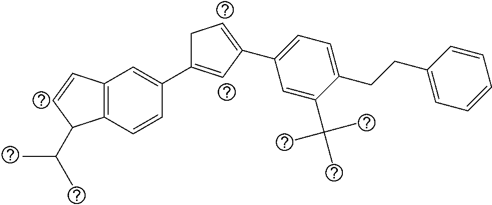 [SMILES: CC(C)C1C=Cc2cc(C3=CC(c4ccc(CCc5ccccc5)c(C(C)(C)C)c4)=CC3)ccc21] | 296 | 5-{3-[4-(benzyloxy)-3- (trifluoromethyl)phenyl]-1,2,4- oxadiazol-5-yl}-1-(propan-2-yl)- 1H-1,2,3-benzotriazole |
| 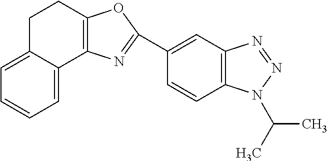 [SMILES: CC(C)n1nnc2cc(-c3nc4c(o3)CCc3ccccc3-4)ccc21] | 297 | 5-{4H,5H-naphtho[2,1- d][1,3]oxazol-2-yl}-1-(propan-2- yl)-1H-1,2,3-benzotriazole |
| 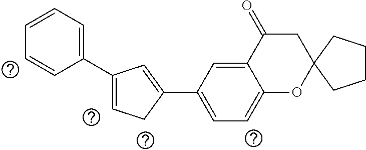 [SMILES: O=C1CC2(CCCC2)Oc2ccc(C3=CC(c4ccccc4)=CC3)cc21] | 298 | 6-[3-(pyridin-3-yl)-1,2,4- oxadiazol-5-yl]-3,4- dihydrospiro[1-benzopyran-2,1′- cyclopentane]-4-one |
| 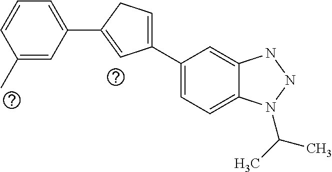 [SMILES: Cc1cccc(C2=CC(c3ccc4c(c3)NNN4C(C)C)=CC2)c1] | 299 | 3-(1-isopropylbenzotriazol-5-yl)- 5-(3-methoxyphenyl)-1,2,4- oxadiazole |
| 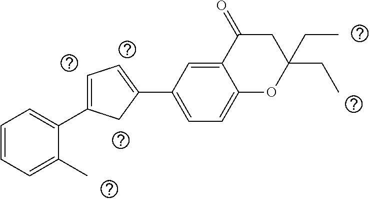 [SMILES: CCC1(CC)CC(=O)c2cc(C3=CC=C(c4ccccc4C)C3)ccc2O1] | 300 | 2,2-diethyl-6-(5-(2- methoxyphenyl)-1,3,4-oxadiazol- 2-yl)chroman-4-one |
| 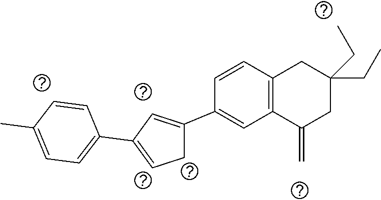 [SMILES: C=C1CC(CC)(CC)Cc2ccc(C3=CC(c4ccc(C)cc4)=CC3)cc21] | 301 | 2,2-diethyl-6-[3-(6-methoxy-3- pyridyl)-1,2,4-oxadiazol-5- yl]chroman-4-one |
| 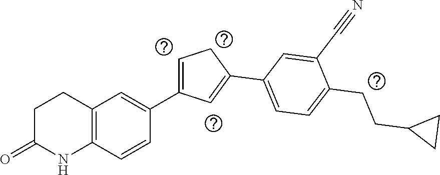 [SMILES: N#Cc1cc(C2=CC(c3ccc4c(c3)CCC(=O)N4)=CC2)ccc1CCC1CC1] | 302 | 2-((cyclopropylmethyl)amino)-5- (3-(2-oxo-1,2,3,4- tetrahydroquinolin-6-yl)-1,2,4- oxadiazol-5-yl)benzonitrile |
| 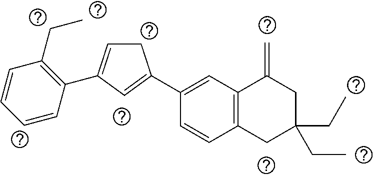 [SMILES: C=C1CC(CC)(CC)Cc2ccc(C3=CC(c4ccccc4CC)=CC3)cc21] | 303 | 2,2-diethyl-6-(3-(4- methoxypyridin-3-yl)-1,2,4- oxadiazol-5-yl)chroman-4-one |
| 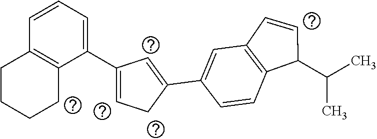 [SMILES: CC(C)C1C=Cc2cc(C3=CC(c4cccc5c4CCCC5)=CC3)ccc21] | 304 | 3-chroman-8-yl-5-(1- isopropylbenzotriazol-5-yl)-1,2,4- oxadiazole |
| 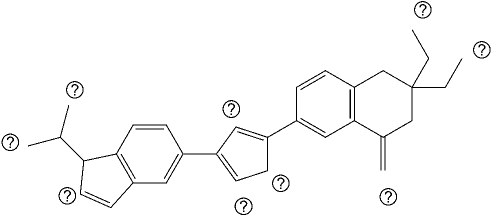 [SMILES: C=C1CC(CC)(CC)Cc2ccc(C3=CC(c4ccc5c(c4)C=CC5C(C)C)=CC3)cc21] | 305 | 2,2-diethyl-6-(3-(1-isopropyl-1H- benzo[d][1,2,3]triazol-5-yl)-1,2,4- oxadiazol-5-yl)chroman-4-one |
| 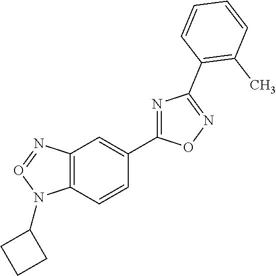 | 306 | 1-cyclobutyl-5-[3-(2- methylphenyl)-1,2,4-oxadiazol-5- yl]-1H-1,2,3-benzotriazole |
| 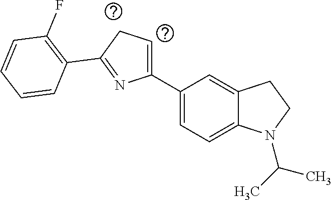 [SMILES: CC(C)N1CCc2cc(C3=CCC(c4ccccc4F)=N3)ccc21] | 307 | 5-(2-fluorophenyl)-3-(1- isopropyl-1H- benzo[d][1,2,3]triazol-5-yl)-1,2,4- oxadiazole |
| 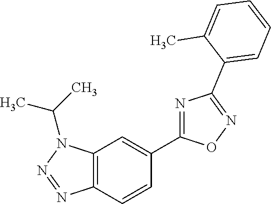 [SMILES: Cc1ccccc1-c1noc(-c2ccc3nnn(C(C)C)c3c2)n1] | 308 | 6-[3-(2-methylphenyl)-1,2,4- oxadiazol-5-yl]-1-(propan-2-yl)- 1H-1,2,3-benzotriazole |
| 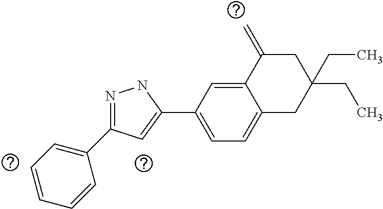 [SMILES: C=C1CC(CC)(CC)Cc2ccc(-c3cc(-c4ccccc4)n[nH]3)cc21] | 309 | 2,2-diethyl-6-(3-(pyridin-3-yl)- 1,2,4-thiadiazol-5-yl)chroman-4- one |
| 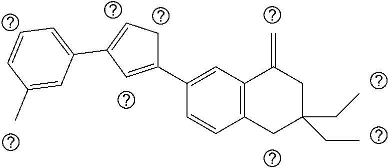 [SMILES: C=C1CC(CC)(CC)Cc2ccc(C3=CC(c4cccc(C)c4)=CC3)cc21] | 310 | 2,2-diethyl-6-[3-(5-hydroxy-3- pyridyl)-1,2,4-oxadiazol-5- yl]chroman-4-one |
| 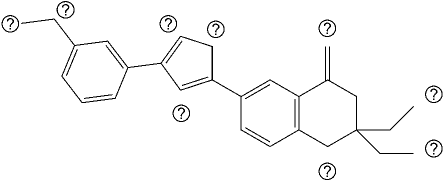 [SMILES: C=C1CC(CC)(CC)Cc2ccc(C3=CC(c4cccc(CC)c4)=CC3)cc21] | 311 | 2,2-diethyl-6-(3-(5- methoxypyridin-3-yl)-1,2,4- oxadiazol-5-yl)chroman-4-one |
| 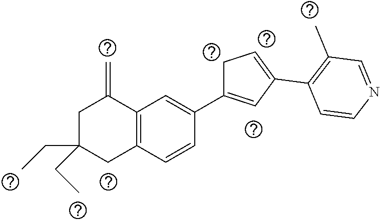 [SMILES: C=C1CC(CC)(CC)Cc2ccc(C3=CC(c4ccncc4C)=CC3)cc21] | 312 | 2,2-diethyl-6-(3-(3- hydroxypyridin-4-yl)-1,2,4- oxadiazol-5-yl)chroman-4-one |
| 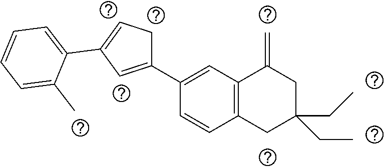 [SMILES: C=C1CC(CC)(CC)Cc2ccc(C3=CC(c4ccccc4C)=CC3)cc21] | 313 | 2,2-diethyl-6-(3-(3- (trifluoromethyl)pyridin-4-yl)- 1,2,4-oxadiazol-5-yl)chroman-4- one |
| 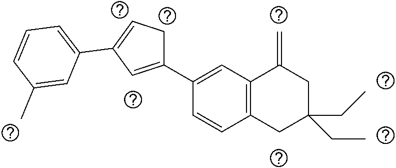 [SMILES: C=C1CC(CC)(CC)Cc2ccc(C3=CC(c4cccc(C)c4)=CC3)cc21] | 314 | 2,2-diethyl-6-(3-(2- (trifluoromethyl)pyridin-4-yl)- 1,2,4-oxadiazol-5-yl)chroman-4- one |
| 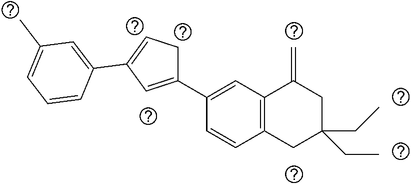 [SMILES: C=C1CC(CC)(CC)Cc2ccc(C3=CC(c4cccc(C)c4)=CC3)cc21] | 315 | 2,2-diethyl-6-(3-(2- methylpyridin-4-yl)-1,2,4- oxadiazol-5-yl)chroman-4-one |
| 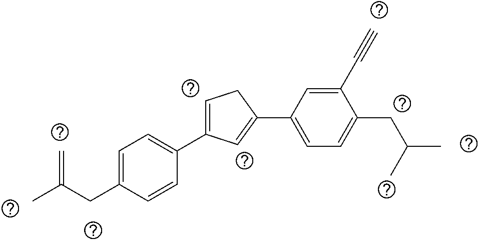 [SMILES: C#Cc1cc(C2=CC(c3ccc(CC(=C)C)cc3)=CC2)ccc1CC(C)C] | 316 | N-(4-(5-(3-cyano-4- (isopropylamino)phenyl)-1,2,4- oxadiazol-3-yl)phenyl)acetamide |
| 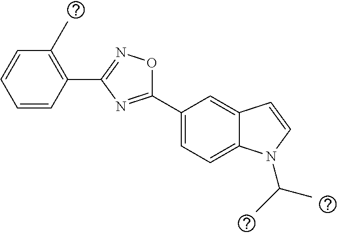 [SMILES: Cc1ccccc1-c1noc(-c2ccc3c(ccn3C(C)C)c2)n1] | 320 | 3-(2-chlorophenyl)-5-(1- isopropyl-1H-indol-5-yl)-1,2,4- oxadiazole |
| 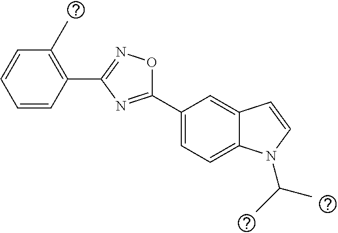 [SMILES: Cc1ccccc1-c1noc(-c2ccc3c(ccn3C(C)C)c2)n1] | 321 | 5-(1-isopropyl-1H-indol-5-yl)-3- (2-(trifluoromethoxy)phenyl)- 1,2,4-oxadiazole |
| 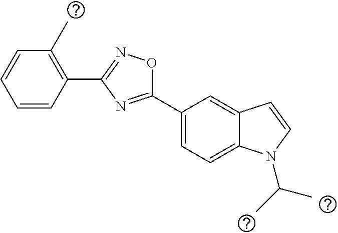 [SMILES: Cc1ccccc1-c1noc(-c2ccc3c(ccn3C(C)C)c2)n1] | 322 | 3-(2-bromophenyl)-5-(1- isopropyl-1H-indol-5-yl)-1,2,4- oxadiazole |
| 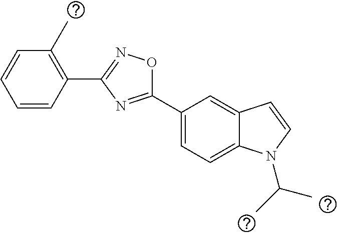 [SMILES: Cc1ccccc1-c1noc(-c2ccc3c(ccn3C(C)C)c2)n1] | 323 | 2-(5-(1-isopropyl-1H-indol-5-yl)- 1,2,4-oxadiazol-3-yl)phenol |
| 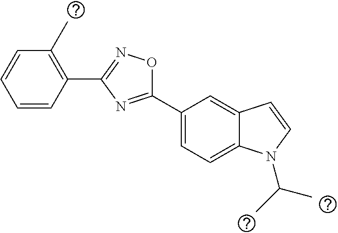 [SMILES: Cc1ccccc1-c1noc(-c2ccc3c(ccn3C(C)C)c2)n1] | 324 | 3-(2-isopropoxyphenyl)-5-(1- isopropyl-1H-indol-5-yl)-1,2,4- oxadiazole |
| 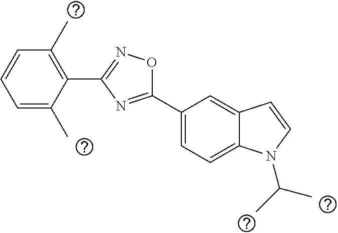 [SMILES: Cc1cccc(C)c1-c1noc(-c2ccc3c(ccn3C(C)C)c2)n1] | 325 | 3-(2,6-dimethoxyphenyl)-5-(1- isopropylindol-5-yl)-1,2,4- oxadiazole |
| 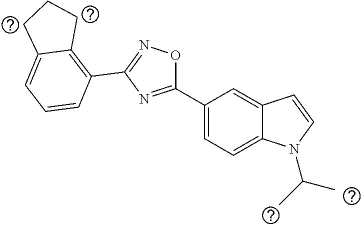 [SMILES: CC(C)n1ccc2cc(-c3nc(-c4cccc5c4CCC5)no3)ccc21] | 326 | 3-(benzo[d][1,3]dioxol-4-yl)-5-(1- isopropyl-1H-indol-5-yl)-1,2,4- oxadiazole |
| 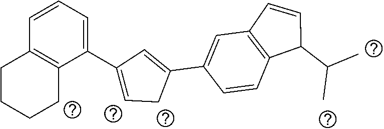 [SMILES: CC(C)C1C=Cc2cc(C3=CC(c4cccc5c4CCCC5)=CC3)ccc21] | 327 | 3-chroman-8-yl-5-(1- isopropylindol-5-yl)-1,2,4- oxadiazole |
| 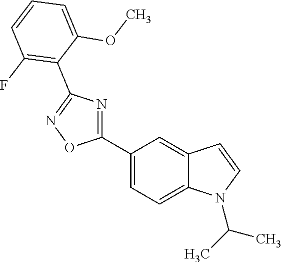 [SMILES: COc1cccc(F)c1-c1noc(-c2ccc3c(ccn3C(C)C)c2)n1] | 328 | 3-(2-fluoro-6-methoxy-phenyl)-5- (1-isopropylindol-5-yl)-1,2,4- oxadiazole |
| 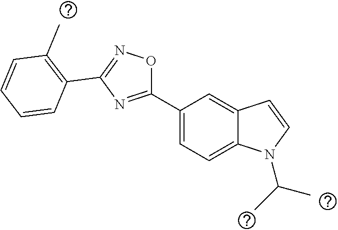 [SMILES: Cc1ccccc1-c1noc(-c2ccc3c(ccn3C(C)C)c2)n1] | 329 | 5-(1-isopropyl-1H-indazol-5-yl)- 3-(o-tolyl)-1,2,4-oxadiazole |
| 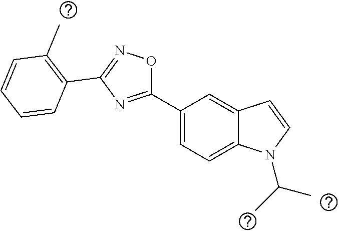 [SMILES: Cc1ccccc1-c1noc(-c2ccc3c(ccn3C(C)C)c2)n1] | 330 | 3-(2-chlorophenyl)-5-(1- isopropyl-1H-indazol-5-yl)-1,2,4- oxadiazole |
| 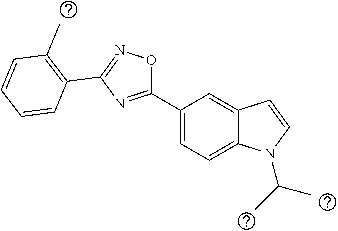 [SMILES: Cc1ccccc1-c1noc(-c2ccc3c(ccn3C(C)C)c2)n1] | 331 | 5-(1-isopropyl-1H-indazol-5-yl)- 3-(2-(trifluoromethoxy)phenyl)- 1,2,4-oxadiazole |
| 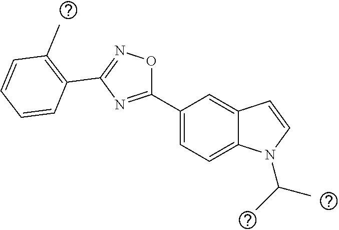 [SMILES: Cc1ccccc1-c1noc(-c2ccc3c(ccn3C(C)C)c2)n1] | 332 | 3-(2-bromophenyl)-5-(1- isopropyl-1H-indazol-5-yl)-1,2,4- oxadiazole |
| 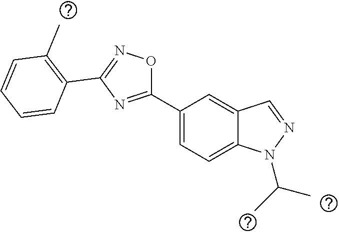 [SMILES: Cc1ccccc1-c1noc(-c2ccc3c(cnn3C(C)C)c2)n1] | 333 | 2-(5-(1-isopropyl-1H-indazol-5- yl)-1,2,4-oxadiazol-3-yl)phenol |
| 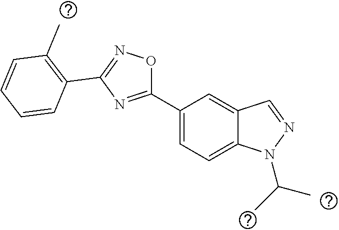 [SMILES: Cc1ccccc1-c1noc(-c2ccc3c(cnn3C(C)C)c2)n1] | 334 | 3-(2-isopropoxyphenyl)-5-(1- isopropyl-1H-indazol-5-yl)-1,2,4- oxadiazole |
| 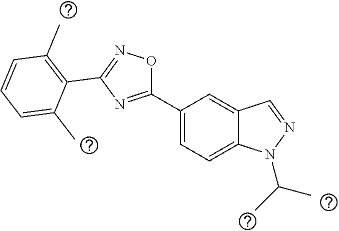 [SMILES: Cc1cccc(C)c1-c1noc(-c2ccc3c(cnn3C(C)C)c2)n1] | 335 | 3-(2,6-dimethoxyphenyl)-5-(1- isopropylindazol-5-yl)-1,2,4- oxadiazole |
| 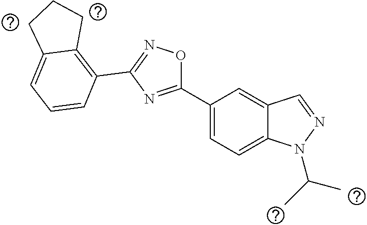 [SMILES: CC(C)n1ncc2cc(-c3nc(-c4cccc5c4CCC5)no3)ccc21] | 336 | 3-(benzo[d][1,3]dioxol-4-yl)-5-(1- isopropyl-1H-indazol-5-yl)-1,2,4- oxadiazole |
| 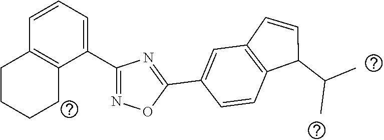 [SMILES: CC(C)C1C=Cc2cc(-c3nc(-c4cccc5c4CCCC5)no3)ccc21] | 337 | 3-chroman-8-yl-5-(1- isopropylindazol-5-yl)-1,2,4- oxadiazole |
| 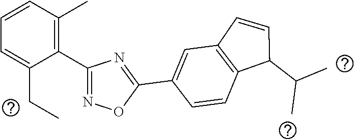 [SMILES: CCc1cccc(C)c1-c1noc(-c2ccc3c(c2)C=CC3C(C)C)n1] | 338 | 3-(2-fluoro-6-methoxy-phenyl)-5- (1-isopropylindazol-5-yl)-1,2,4- oxadiazole |
| 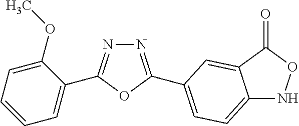 [SMILES: COc1ccccc1-c1nnc(-c2ccc3[nH]oc(=O)c3c2)o1] | 339 | 5-[5-(2-methoxyphenyl)-1,3,4- oxadiazol-2-yl]-1,3-dihydro-2,1- benzoxazol-3-one |
| 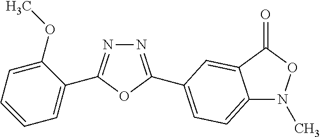 [SMILES: COc1ccccc1-c1nnc(-c2ccc3c(c2)c(=O)on3C)o1] | 340 | 5-[5-(2-methoxyphenyl)-1,3,4- oxadiazol-2-yl]-1-methyl-1,3- dihydro-2,1-benzoxazol-3-one |
| 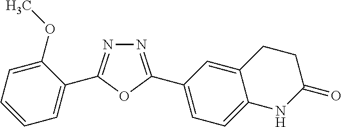 [SMILES: COc1ccccc1-c1nnc(-c2ccc3c(c2)CCC(=O)N3)o1] | 341 | 6-[5-(2-methoxyphenyl)-1,3,4- oxadiazol-2-yl]-1,2,3,4- tetrahydroquinolin-2-one |
| 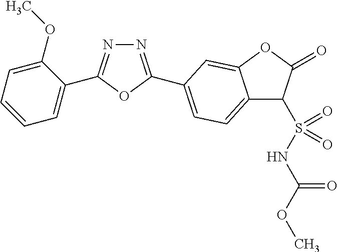 [SMILES: COC(=O)NS(=O)(=O)C1C(=O)Oc2cc(-c3nnc(-c4ccccc4OC)o3)ccc21] | 342 | methyl N-({6-[5-(2- methoxyphenyl)-1,3,4-oxadiazol- 2-yl]-2-oxo-2,3-dihydro-1,3- benzoxazol-3- yl}sulfonyl)carbamate |
| 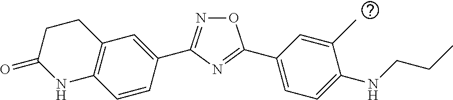 [SMILES: CCCNc1ccc(-c2nc(-c3ccc4c(c3)CCC(=O)N4)no2)cc1C] | 343 | 2-[(2-fluoroethyl)amino]-5-[3-(2- oxo-1,2,3,4-tetrahydroquinolin-6- yl)-1,2,4-oxadiazol-5- yl]benzonitrile |
| 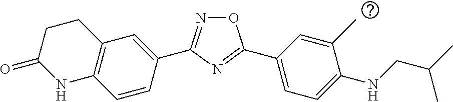 [SMILES: Cc1cc(-c2nc(-c3ccc4c(c3)CCC(=O)N4)no2)ccc1NCC(C)C] | 344 | 2-[(2,2-difluoroethyl)amino]-5-[3- (2-oxo-1,2,3,4- tetrahydroquinolin-6-yl)-1,2,4- oxadiazol-5-yl]benzonitrile |
| 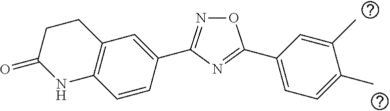 [SMILES: Cc1ccc(-c2nc(-c3ccc4c(c3)CCC(=O)N4)no2)cc1C] | 345 | 2-amino-5-[3-(2-oxo-1,2,3,4- tetrahydroquinolin-6-yl)-1,2,4- oxadiazol-5-yl]benzonitrile |
| 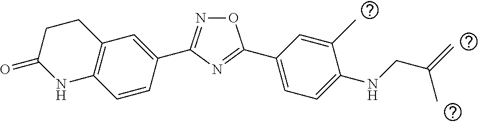 [SMILES: C=C(C)CNc1ccc(-c2nc(-c3ccc4c(c3)CCC(=O)N4)no2)cc1C] | 346 | 2-[(2-fluoroprop-2-en-1- yl)amino]-5-[3-(2-oxo-1,2,3,4- tetrahydroquinolin-6-yl)-1,2,4- oxadiazol-5-yl]benzonitrile |
| 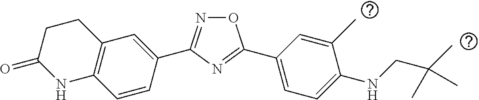 [SMILES: Cc1cc(-c2nc(-c3ccc4c(c3)CCC(=O)N4)no2)ccc1NCC(C)(C)C] | 347 | 2-[(2,2-difluoropropyl)amino]-5- [3-(2-oxo-1,2,3,4- tetrahydroquinolin-6-yl)-1,2,4- oxadiazol-5-yl]benzonitrile |
| 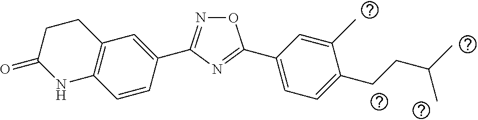 [SMILES: Cc1cc(-c2nc(-c3ccc4c(c3)CCC(=O)N4)no2)ccc1CCC(C)C] | 348 | 2-[(2-fluoropropyl)amino]-5-[3- (2-oxo-1,2,3,4- tetrahydroquinolin-6-yl)-1,2,4- oxadiazol-5-yl]benzonitrile |
| 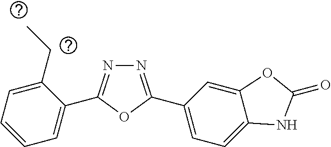 [SMILES: CCc1ccccc1-c1nnc(-c2ccc3[nH]c(=O)oc3c2)o1] | 349 | 6-[5-(2-methoxyphenyl)-1,3,4- oxadiazol-2-yl]-2,3-dihydro-1,3- benzoxazol-2-one |
| 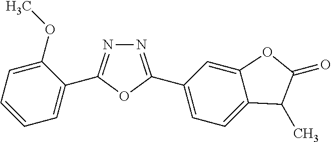 [SMILES: COc1ccccc1-c1nnc(-c2ccc3c(c2)OC(=O)C3C)o1] | 350 | 6-[5-(2-methoxyphenyl)-1,3,4- oxadiazol-2-yl]-3-methyl-2,3- dihydro-1,3-benzoxazol-2-one |
| 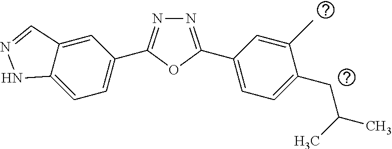 [SMILES: Cc1cc(-c2nnc(-c3ccc4[nH]ncc4c3)o2)ccc1CC(C)C] | 351 | 5-[5-(1H-indazol-5-yl)-1,3,4- oxadiazol-2-yl]-2-[(propan-2- yl)amino]benzonitrile |
| 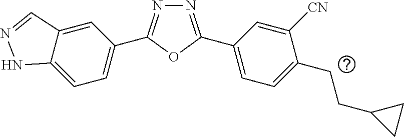 [SMILES: N#Cc1cc(-c2nnc(-c3ccc4[nH]ncc4c3)o2)ccc1CCC1CC1] | 352 | 2-[(cyclopropylmethyl)amino]-5- [5-(1H-indazol-5-yl)-1,3,4- oxadiazol-2-yl]benzonitrile |
| 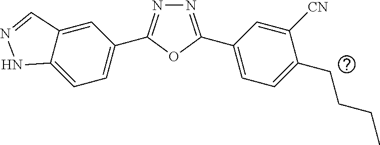 [SMILES: CCCCc1ccc(-c2nnc(-c3ccc4[nH]ncc4c3)o2)cc1C#N] | 353 | 2-[(2-fluoroethyl)amino]-5-[5- (1H-indazol-5-yl)-1,3,4- oxadiazol-2-yl]benzonitrile |
| 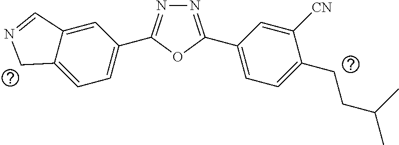 [SMILES: CC(C)CCc1ccc(-c2nnc(-c3ccc4c(c3)C=NC4)o2)cc1C#N] | 354 | 2-[(2,2-difluoroethyl)amino]-5-[5- (1H-indazol-5-yl)-1,3,4- oxadiazol-2-yl]benzonitrile |
| 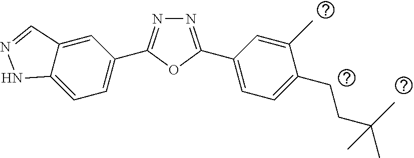 [SMILES: Cc1cc(-c2nnc(-c3ccc4[nH]ncc4c3)o2)ccc1CCC(C)(C)C] | 355 | 2-[(2,2-difluoropropyl)amino]-5- [5-(1H-indazol-5-yl)-1,3,4- oxadiazol-2-yl]benzonitrile |
| 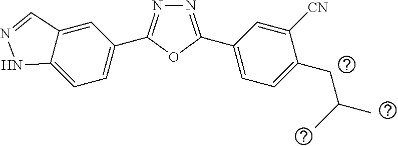 [SMILES: CC(C)Cc1ccc(-c2nnc(-c3ccc4[nH]ncc4c3)o2)cc1C#N] | 356 | 5-[5-(1H-1,3-benzodiazol-5-yl)- 1,3,4-oxadiazol-2-yl]-2-[(propan- 2-yl)amino]benzonitrile |
| 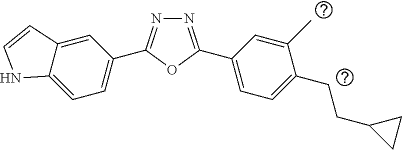 [SMILES: Cc1cc(-c2nnc(-c3ccc4[nH]ccc4c3)o2)ccc1CCC1CC1] | 357 | 5-[5-(1H-1,3-benzodiazol-5-yl)- 1,3,4-oxadiazol-2-yl]-2- [(cyclopropylmethyl)amino] benzonitrile |
| 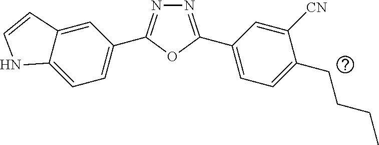 [SMILES: CCCCc1ccc(-c2nnc(-c3ccc4[nH]ccc4c3)o2)cc1C#N] | 358 | 5-[5-(1H-1,3-benzodiazol-5-yl)- 1,3,4-oxadiazol-2-yl]-2-[(2- fluoroethyl)amino]benzonitrile |
| 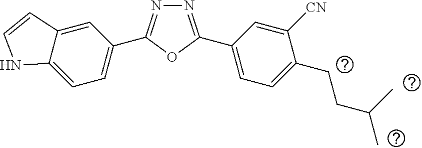 [SMILES: CC(C)CCc1ccc(-c2nnc(-c3ccc4[nH]ccc4c3)o2)cc1C#N] | 359 | 5-[5-(1H-1,3-benzodiazol-5-yl)- 1,3,4-oxadiazol-2-yl]-2-[(2,2- difluoroethyl)amino]benzonitrile |
| 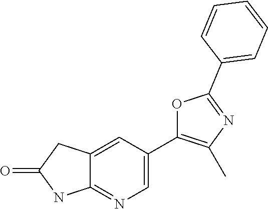 [SMILES: Cc1nc(-c2ccccc2)oc1-c1cnc2c(c1)CC(=O)N2] | 360 | 5-(4-methyl-2-phenyl-1,3-oxazol- 5-yl)-1H,2H,3H-pyrrolo[2,3- b]pyridin-2-one |
| 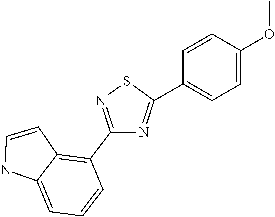 [SMILES: COc1ccc(-c2nc(-c3cccc4[nH]ccc34)ns2)cc1] | 361 | 4-[5-(4-methoxyphenyl)-1,2,4- thiadiazol-3-yl]-1H-indole |
| 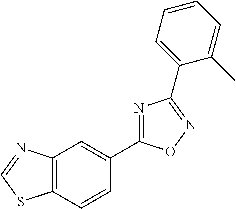 [SMILES: Cc1ccccc1-c1noc(-c2ccc3scnc3c2)n1] | 362 | 5-[3-(2-methylphenyl)-1,2,4- oxadiazol-5-yl]-1,3- benzothiazole |
| 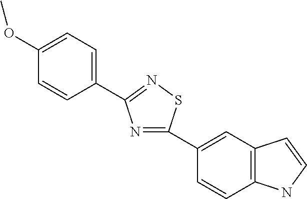 [SMILES: COc1ccc(-c2nsc(-c3ccc4[nH]ccc4c3)n2)cc1] | 363 | 5-[3-(4-methoxyphenyl)-1,2,4- thiadiazol-5-yl]-1H-indole |
| 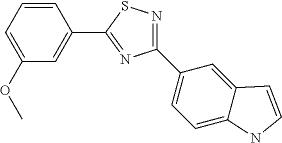 [SMILES: COc1cccc(-c2nc(-c3ccc4[nH]ccc4c3)ns2)c1] | 364 | 5-[5-(3-methoxyphenyl)-1,2,4- thiadiazol-3-yl]-1H-indole |
| 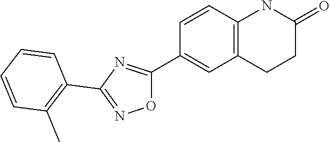 [SMILES: Cc1ccccc1-c1noc(-c2ccc3c(c2)CCC(=O)N3)n1] | 365 | 6-[3-(2-methylphenyl)-1,2,4- oxadiazol-5-yl]-1,2,3,4- tetrahydroquinolin-2-one |
| 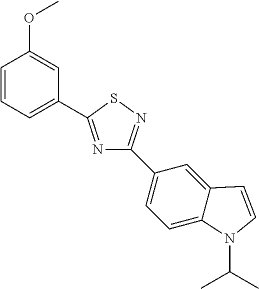 [SMILES: COc1cccc(-c2nc(-c3ccc4c(ccn4C(C)C)c3)ns2)c1] | 366 | 5-[5-(3-methoxyphenyl)-1,2,4- thiadiazol-3-yl]-1-(propan-2-yl)- 1H-indole |
| 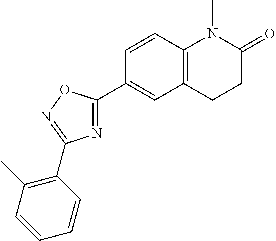 [SMILES: Cc1ccccc1-c1noc(-c2ccc3c(c2)CCC(=O)N3C)n1] | 367 | 1-methyl-6-[3-(2-methylphenyl)- 1,2,4-oxadiazol-5-yl]-1,2,3,4- tetrahydroquinolin-2-one |
| 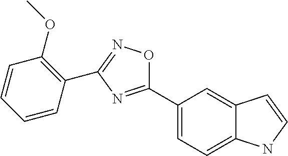 [SMILES: COc1ccccc1-c1noc(-c2ccc3[nH]ccc3c2)n1] | 368 | 5-(1H-indol-5-yl)-3-(2- methoxyphenyl)-1,2,4-oxadiazole |
| 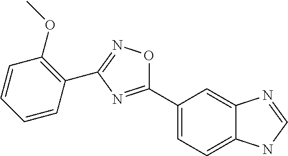 [SMILES: COc1ccccc1-c1noc(-c2ccc3[nH]cnc3c2)n1] | 369 | 5-(1H-benzo[d]imidazol-5-yl)-3- (2-methoxyphenyl)-1,2,4- oxadiazole |
| 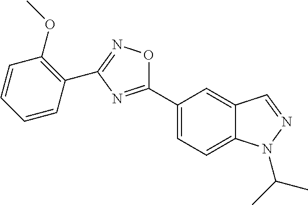 [SMILES: COc1ccccc1-c1noc(-c2ccc3c(cnn3C(C)C)c2)n1] | 370 | 5-(1-isopropyl-1H-indazol-5-yl)- 3-(2-methoxyphenyl)-1,2,4- oxadiazole |
| 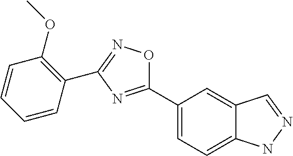 [SMILES: COc1ccccc1-c1noc(-c2ccc3[nH]ncc3c2)n1] | 371 | 5-(1H-indazol-5-yl)-3-(2- methoxyphenyl)-1,2,4-oxadiazole |
| 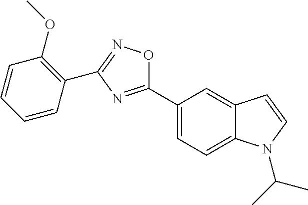 [SMILES: COc1ccccc1-c1noc(-c2ccc3c(ccn3C(C)C)c2)n1] | 372 | 5-(1-isopropyl-1H-indol-5-yl)-3- (2-methoxyphenyl)-1,2,4- oxadiazole |
| 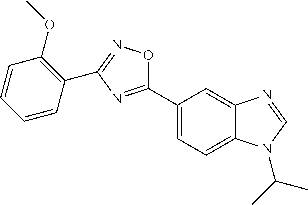 [SMILES: COc1ccccc1-c1noc(-c2ccc3c(c2)ncn3C(C)C)n1] | 373 | 5-(1-isopropyl-1H- benzo[d]imidazol-5-yl)-3-(2- methoxyphenyl)-1,2,4-oxadiazole |
| 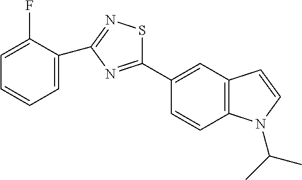 [SMILES: CC(C)n1ccc2cc(-c3nc(-c4ccccc4F)ns3)ccc21] | 374 | 5-[3-(2-fluorophenyl)-1,2,4- thiadiazol-5-yl]-1-(propan-2-yl)- 1H-indole |
| 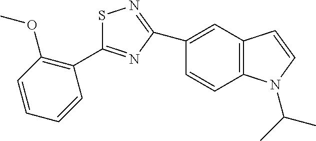 [SMILES: COc1ccccc1-c1nc(-c2ccc3c(ccn3C(C)C)c2)ns1] | 375 | 5-[5-(2-methoxyphenyl)-1,2,4- thiadiazol-3-yl]-1-(propan-2-yl)- 1H-indole |
| 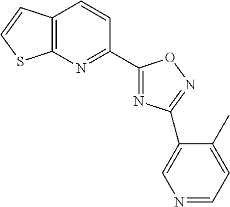 [SMILES: Cc1ccncc1-c1noc(-c2ccc3ccsc3n2)n1] | 376 | 4-methyl-3-(5-{thieno[2,3- b]pyridin-6-yl}-1,2,4-oxadiazol- 3-yl)pyridine |
| 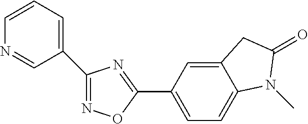 [SMILES: CN1C(=O)Cc2cc(-c3nc(-c4cccnc4)no3)ccc21] | 377 | 1-methyl-5-[3-(pyridin-3-yl)- 1,2,4-oxadiazol-5-yl]-2,3- dihydro-1H-indol-2-one |
| 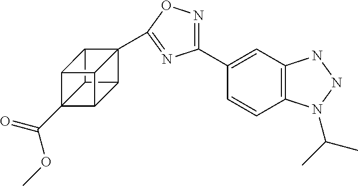 [SMILES: COC(=O)C12C3C4C1C1C2C3C41c1nc(-c2ccc3c(c2)NNN3C(C)C)no1] | 378 | 8-{3-[1-(propan-2-yl)-1H-1,2,3- benzotriazol-5-yl]-1,2,4- oxadiazol-5-y1}cubane-1- carboxylate |
| 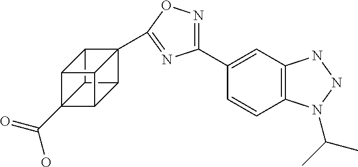 [SMILES: CC(C)N1NNc2cc(-c3noc(C45C6C7C4C4C5C6C74C(=O)O)n3)ccc21] | 379 | 8-{3-[1-(propan-2-yl)-1H-1,2,3- benzotriazol-5-yl]-1,2,4- oxadiazol-5-yl}cubane-1- carboxylic acid |
| 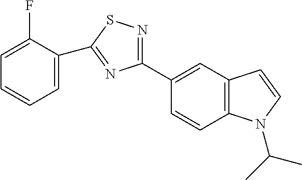 [SMILES: CC(C)n1ccc2cc(-c3nsc(-c4ccccc4F)n3)ccc21] | 380 | 5-[5-(2-fluorophenyl)-1,2,4- thiadiazol-3-yl]-1-(propan-2-yl)- 1H-indole |
| 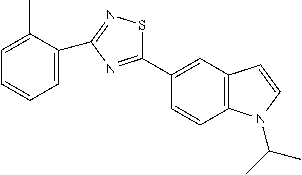 [SMILES: Cc1ccccc1-c1nsc(-c2ccc3c(ccn3C(C)C)c2)n1] | 381 | 5-[3-(2-methylphenyl)-1,2,4- thiadiazol-5-yl]-1-(propan-2-yl)- 1H-indole |
| 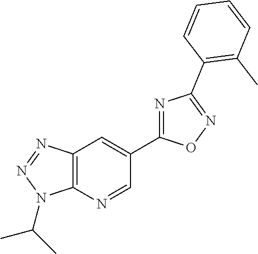 [SMILES: Cc1ccccc1-c1noc(-c2cnc3c(c2)nnn3C(C)C)n1] | 382 | 3-(2-methylphenyl)-5-[3-(propan- 2-yl)-3H-[1,2,3]triazolo[4,5- b]pyridin-6-yl]-1,2,4-oxadiazole |
| 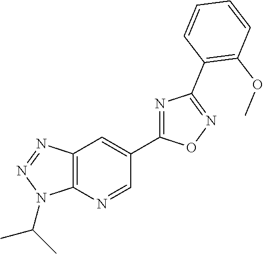 [SMILES: COc1ccccc1-c1noc(-c2cnc3c(c2)nnn3C(C)C)n1] | 383 | 3-(2-methoxyphenyl)-5-[3- (propan-2-yl)-3H- [1,2,3]triazolo[4,5-b]pyridin-6- yl]-1,2,4-oxadiazole |
| 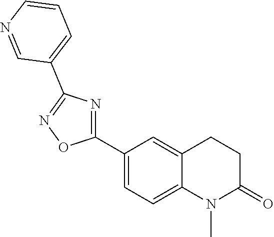 [SMILES: CN1C(=O)CCc2cc(-c3nc(-c4cccnc4)no3)ccc21] | 384 | 1-methyl-6-[3-(pyridin-3-yl)- 1,2,4-oxadiazol-5-yl]-1,2,3,4- tetrahydroquinolin-2-one |
| 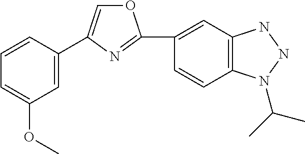 [SMILES: COc1cccc(-c2coc(-c3ccc4c(c3)NNN4C(C)C)n2)c1] | 385 | 5-[4-(3-methoxyphenyl)-1,3- oxazol-2-yl]-1-(propan-2-yl)-1H- 1,2,3-benzotriazole |
| 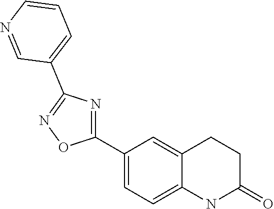 [SMILES: O=C1CCc2cc(-c3nc(-c4cccnc4)no3)ccc2N1] | 386 | 6-[3-(pyridin-3-yl)-1,2,4- oxadiazol-5-yl]-1,2,3,4- tetrahydroquinolin-2-one |
| 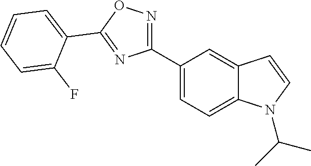 [SMILES: CC(C)n1ccc2cc(-c3noc(-c4ccccc4F)n3)ccc21] | 387 | 5-(2-fluorophenyl)-3-(1- isopropyl-1H-indol-5-yl)-1,2,4- oxadiazole |
| 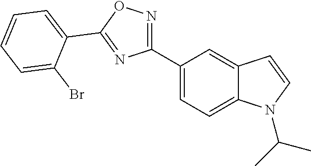 [SMILES: CC(C)n1ccc2cc(-c3noc(-c4ccccc4Br)n3)ccc21] | 388 | 5-(2-bromophenyl)-3-(1- isopropyl-1H-indol-5-yl)-1,2,4- oxadiazole |
| 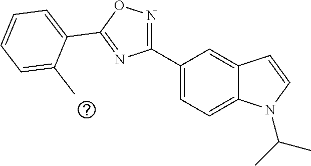 [SMILES: Cc1ccccc1-c1nc(-c2ccc3c(ccn3C(C)C)c2)no1] | 389 | 3-(1-isopropyl-1H-indol-5-yl)-5- (o-tolyl)-1,2,4-oxadiazole |
| 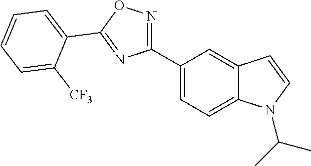 [SMILES: CC(C)n1ccc2cc(-c3noc(-c4ccccc4C(F)(F)F)n3)ccc21] | 390 | 3-(1-isopropyl-1H-indol-5-yl)-5- (2-(trifluoromethyl)phenyl)-1,2,4- oxadiazole |
| 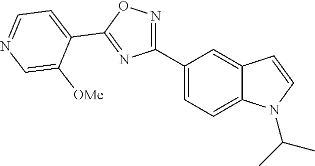 [SMILES: COc1cnccc1-c1nc(-c2ccc3c(ccn3C(C)C)c2)no1] | 391 | 3-(1-isopropyl-1H-indol-5-yl)-5- (3-methoxypyridin-4-yl)-1,2,4- oxadiazole |
| 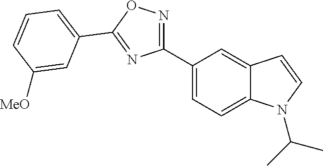 [SMILES: COc1cccc(-c2nc(-c3ccc4c(ccn4C(C)C)c3)no2)c1] | 392 | 3-(1-isopropyl-1H-indol-5-yl)-5- (3-methoxyphenyl)-1,2,4- oxadiazole |
| 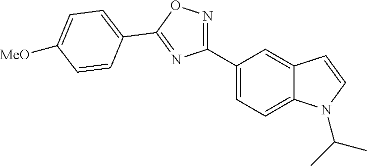 [SMILES: COc1ccc(-c2nc(-c3ccc4c(ccn4C(C)C)c3)no2)cc1] | 393 | 3-(1-isopropyl-1H-indol-5-yl)-5- (4-methoxyphenyl)-1,2,4- oxadiazole |
| 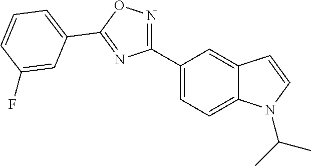 [SMILES: CC(C)n1ccc2cc(-c3noc(-c4cccc(F)c4)n3)ccc21] | 394 | 5-(3-fluorophenyl)-3-(1- isopropyl-1H-indol-5-yl)-1,2,4- oxadiazole |
| 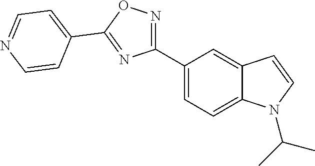 [SMILES: CC(C)n1ccc2cc(-c3noc(-c4ccncc4)n3)ccc21] | 395 | 3-(1-isopropyl-1H-indol-5-yl)-5- (pyridin-4-yl)-1,2,4-oxadiazole |
| 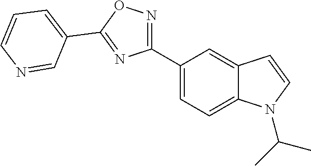 [SMILES: CC(C)n1ccc2cc(-c3noc(-c4cccnc4)n3)ccc21] | 396 | 3-(1-isopropyl-1H-indol-5-yl)-5- (pyridin-3-yl)-1,2,4-oxadiazole |
| 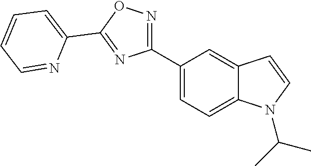 [SMILES: CC(C)n1ccc2cc(-c3noc(-c4ccccn4)n3)ccc21] | 397 | 3-(1-isopropyl-1H-indol-5-yl)-5- (pyridin-2-yl)-1,2,4-oxadiazole |
| 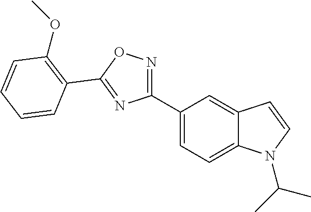 [SMILES: COc1ccccc1-c1nc(-c2ccc3c(ccn3C(C)C)c2)no1] | 398 | 3-(1-isopropyl-1H-indol-5-yl)-5- (2-methoxyphenyl)-1,2,4- oxadiazole |
| 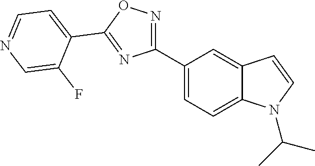 [SMILES: CC(C)n1ccc2cc(-c3noc(-c4ccncc4F)n3)ccc21] | 399 | 5-(3-fluoropyridin-4-yl)-3-(1- isopropyl-1H-indol-5-yl)-1,2,4- oxadiazole |
| 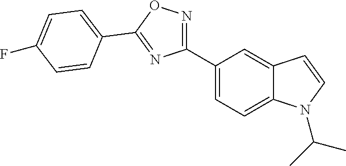 [SMILES: CC(C)n1ccc2cc(-c3noc(-c4ccc(F)cc4)n3)ccc21] | 400 | 5-(4-fluorophenyl)-3-(1- isopropyl-1H-indol-5-yl)-1,2,4- oxadiazole |
| 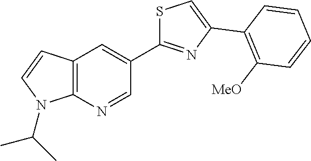 [SMILES: COc1ccccc1-c1csc(-c2cnc3c(ccn3C(C)C)c2)n1] | 401 | 2-(1-isopropyl-1H-pyrrolo[2,3- b]pyridin-5-yl)-4-(2- methoxyphenyl)thiazole |
| 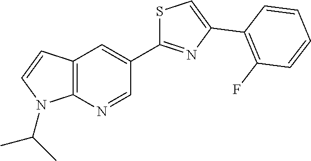 [SMILES: CC(C)n1ccc2cc(-c3nc(-c4ccccc4F)cs3)cnc21] | 402 | 4-(2-fluorophenyl)-2-(1- isopropyl-1H-pyrrolo[2,3- b]pyridin-5-yl)thiazole |
| 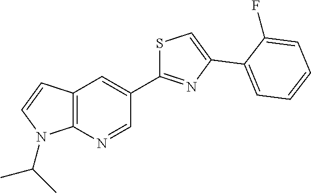 [SMILES: CC(C)n1ccc2cc(-c3nc(-c4ccccc4F)cs3)cnc21] | 403 | 4-(2-fluorophenyl)-2-(1- isopropylindol-5-yl)thiazole |
| 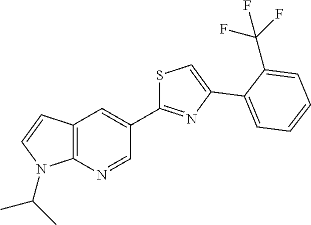 [SMILES: CC(C)n1ccc2cc(-c3nc(-c4ccccc4C(F)(F)F)cs3)cnc21] | 404 | 2-(1-isopropylindol-5-yl)-4-[2- (trifluoromethyl)phenyl]thiazole |
| 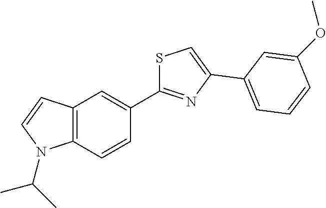 [SMILES: COc1cccc(-c2csc(-c3ccc4c(ccn4C(C)C)c3)n2)c1] | 405 | 2-(1-isopropylindol-5-yl)-4-(3- methoxyphenyl)thiazole |
| 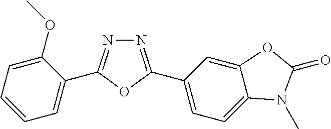 [SMILES: COc1ccccc1-c1nnc(-c2ccc3c(c2)oc(=O)n3C)o1] | 406 | 6-[5-(2-methoxyphenyl)-1,3,4- oxadiazol-2-yl]-3-methyl-2,3- dihydro-1,3-benzoxazol-2-one |
| 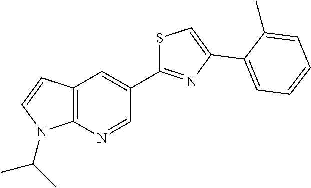 [SMILES: Cc1ccccc1-c1csc(-c2cnc3c(ccn3C(C)C)c2)n1] | 407 | 2-(1-isopropylpyrrolo[2,3- b]pyridin-5-yl)-4-(o-tolyl)thiazole |
| 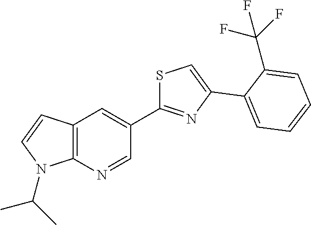 [SMILES: CC(C)n1ccc2cc(-c3nc(-c4ccccc4C(F)(F)F)cs3)cnc21] | 408 | 2-(1-isopropylpyrrolo[2,3- b]pyridin-5-yl)-4-[2- (trifluoromethyl)phenyl]thiazole |
| 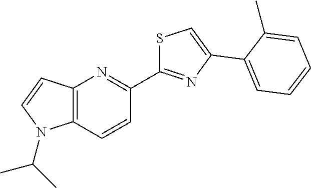 [SMILES: Cc1ccccc1-c1csc(-c2ccc3c(ccn3C(C)C)n2)n1] | 409 | 2-(1-isopropylpyrrolo[3,2- b]pyridin-5-yl)-4-(o-tolyl)lhiazole |
| 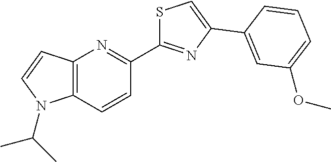 [SMILES: COc1cccc(-c2csc(-c3ccc4c(ccn4C(C)C)n3)n2)c1] | 410 | 2-(1-isopropylpyrrolo[3,2- b]pyridin-5-yl)-4-(3- methoxyphenyl)thiazole |
| 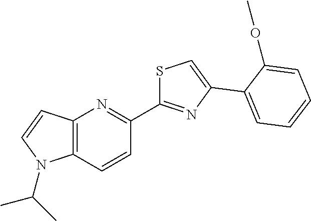 [SMILES: COc1ccccc1-c1csc(-c2ccc3c(ccn3C(C)C)n2)n1] | 411 | 2-(1-isopropyl-1H-pyrrolo[3,2- b]pyridin-5-yl)-4-(2- methoxyphenyl)thiazole |
| 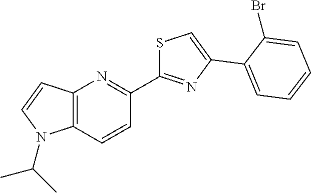 [SMILES: CC(C)n1ccc2nc(-c3nc(-c4ccccc4Br)cs3)ccc21] | 412 | 4-(2-bromophenyl)-2-(1- isopropyl-1H-pyrrolo[3,2- b]pyridin-5-yl)thiazole |
| 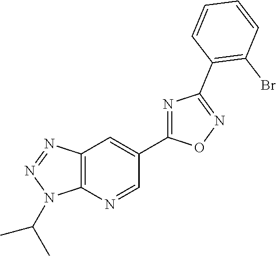 [SMILES: CC(C)n1nnc2cc(-c3nc(-c4ccccc4Br)no3)cnc21] | 413 | 3-(2-bromophenyl)-5-[3-(propan- 2-yl)-3H-[1,2,3]triazolo[4,5- b]pyridin-6-yl]-1,2,4-oxadiazole |
| 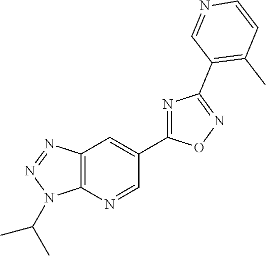 [SMILES: Cc1ccncc1-c1noc(-c2cnc3c(c2)nnn3C(C)C)n1] | 414 | 4-methyl-3-{5-[3-(propan-2-yl)- 3H-[1,2,3]triazolo[4,5-b]pyridin- 6-yl]-1,2,4-oxadiazol-3- yl}pyridine |
| 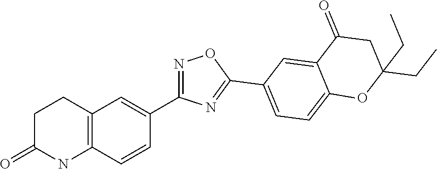 [SMILES: CCC1(CC)CC(=O)c2cc(-c3nc(-c4ccc5c(c4)CCC(=O)N5)no3)ccc2O1] | 415 | 6-(5-(2,2-diethyl-4-oxochroman- 6-yl)-1,2,4-oxadiazol-3-yl)-3,4- dihydroquinolin-2(1H)-one |
| 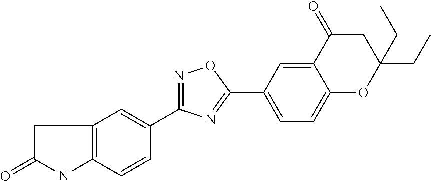 [SMILES: CCC1(CC)CC(=O)c2cc(-c3nc(-c4ccc5c(c4)CC(=O)N5)no3)ccc2O1] | 416 | 5-(5-(2,2-diethyl-4-oxochroman- 6-yl)-1,2,4-oxadiazol-3- yl)indolin-2-one |
| 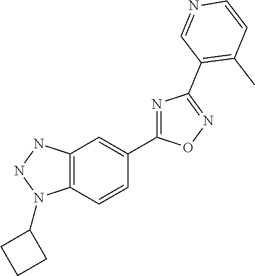 [SMILES: Cc1ccncc1-c1noc(-c2ccc3c(c2)NNN3C2CCC2)n1] | 417 | 1-cyclobutyl-5-[3-(4- methylpyridin-3-yl)-1,2,4- oxadiazol-5-yl]-1H-1,2,3- benzotriazole |
| 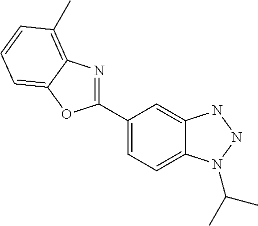 [SMILES: Cc1cccc2oc(-c3ccc4c(c3)NNN4C(C)C)nc12] | 418 | 2-(1-isopropyl-1H- benzo[d][1,2,3]triazol-5-yl)-4- methylbenzo[d]oxazole |
| 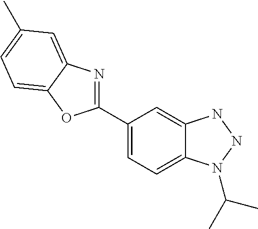 [SMILES: Cc1ccc2oc(-c3ccc4c(c3)NNN4C(C)C)nc2c1] | 419 | 2-(1-isopropyl-1H- benzo[d][1,2,3]triazol-5-yl)-5- methylbenzo[d]oxazole |
| 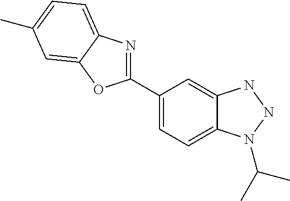 [SMILES: Cc1ccc2nc(-c3ccc4c(c3)NNN4C(C)C)oc2c1] | 420 | 2-(1-isopropyl-1H- benzo[d][1,2,3]triazol-5-yl)-6- methylbenzo[d]oxazole |
| 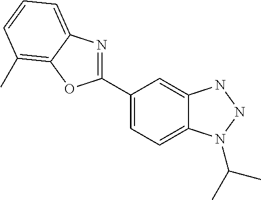 [SMILES: Cc1cccc2nc(-c3ccc4c(c3)NNN4C(C)C)oc12] | 421 | 2-(1-isopropyl-1H- benzo[d][1,2,3]triazol-5-yl)-7- methylbenzo[d]oxazole |
| 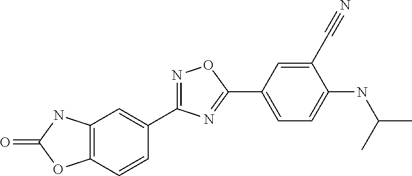 [SMILES: CC(C)Nc1ccc(-c2nc(-c3ccc4oc(=O)[nH]c4c3)no2)cc1C#N] | 422 | 2-(isopropylamino)-5-(3-(2-oxo- 2,3-dihydrobenzo[d]oxazol-5-yl)- 1,2,4-oxadiazol-5-yl)benzonitrile |
| 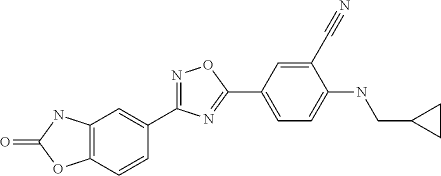 [SMILES: N#Cc1cc(-c2nc(-c3ccc4oc(=O)[nH]c4c3)no2)ccc1NCC1CC1] | 423 | 2-((cyclopropylmethyl)amino)-5- (3-(2-oxo-2,3- dihydrobenzo[d]oxazol-5-yl)- 1,2,4-oxadiazol-5-yl)benzonitrile |
| 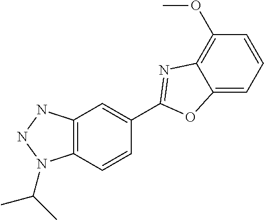 [SMILES: COc1cccc2oc(-c3ccc4c(c3)NNN4C(C)C)nc12] | 424 | 2-(1-isopropyl-1H- benzo[d][1,2,3]triazol-5-yl)-4- methoxybenzo[d]oxazole |
| 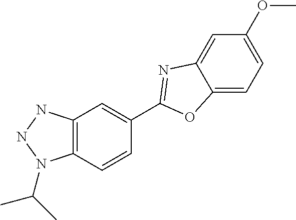 [SMILES: COc1ccc2oc(-c3ccc4c(c3)NNN4C(C)C)nc2c1] | 425 | 2-(1-isopropyl-1H- benzo[d][1,2,3]triazol-5-yl)-5- methoxybenzo[d]oxazole |
| 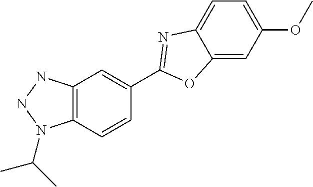 [SMILES: COc1ccc2nc(-c3ccc4c(c3)NNN4C(C)C)oc2c1] | 426 | 2-(1-isopropyl-1H- benzo[d][1,2,3]triazol-5-yl)-6- methoxybenzo[d]oxazole |
| 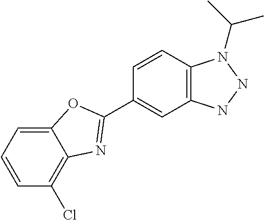 [SMILES: CC(C)N1NNc2cc(-c3nc4c(Cl)cccc4o3)ccc21] | 427 | 4-chloro-2-(1-isopropyl-1H- benzo[d][1,2,3]triazol-5- yl)benzo[d]oxazole |
| 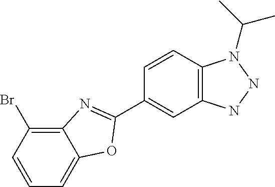 [SMILES: CC(C)N1NNc2cc(-c3nc4c(Br)cccc4o3)ccc21] | 428 | 4-bromo-2-(1- isopropylbenzotriazol-5-yl)-1,3- benzoxazole |
| 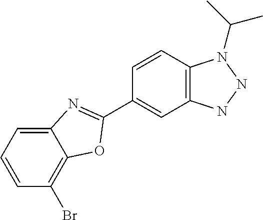 [SMILES: CC(C)N1NNc2cc(-c3nc4cccc(Br)c4o3)ccc21] | 429 | 7-bromo-2-(1-isopropyl-1H- benzo[d][1,2,3]triazol-5- yl)benzo[d]oxazole |
| 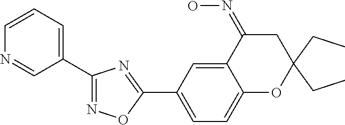 [SMILES: CCC1(CC)C/C(=N/O)c2cc(-c3nc(-c4cccnc4)no3)ccc2O1] | 430 | N-[(4Z)-2,2-diethyl-6-[3-(pyridin- 3-yl)-1,2,4-oxadiazol-5-yl]-3,4- dihydro-2H-1-benzopyran-4- ylidene]hydroxylamine |
| 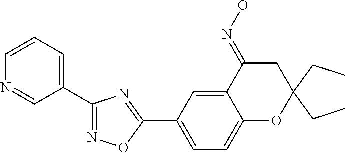 [SMILES: CCC1(CC)C/C(=N\O)c2cc(-c3nc(-c4cccnc4)no3)ccc2O1] | 431 | N-[(4E)-2,2-diethyl-6-[3-(pyridin- 3-yl)-1,2,4-oxadiazol-5-yl]-3,4- dihydro-2H-1-benzopyran-4- ylidene]hydroxylamine |
| 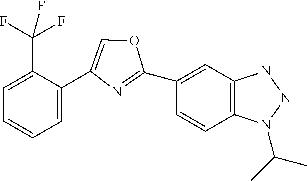 [SMILES: CC(C)N1NNc2cc(-c3nc(-c4ccccc4C(F)(F)F)co3)ccc21] | 432 | 1-(propan-2-yl)-5-{4-[2- (trifluoromethyl)phenyl]-1,3- oxazol-2-yl}-1H-1,2,3- benzotriazole |
| 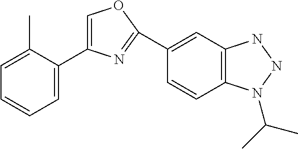 [SMILES: Cc1ccccc1-c1coc(-c2ccc3c(c2)NNN3C(C)C)n1] | 433 | 5-[4-(2-methylphenyl)-1,3- oxazol-2-yl]-1-(propan-2-yl)-1H- 1,2,3-benzotriazole |
| 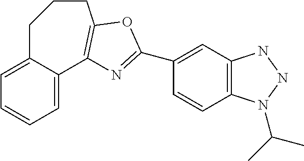 [SMILES: CC(C)N1NNc2cc(-c3nc4c(o3)CCCc3ccccc3-4)ccc21] | 434 | 4-[1-(propan-2-yl)-1H-1,2,3- benzotriazol-5-yl]-5-oxa-3- azatricyclo[8.4.0.0²,6]tetradeca- 1(14),2(6),3,10,12-pentaene |
| 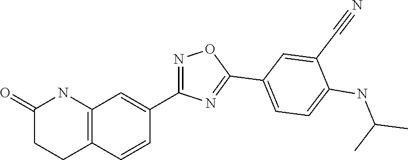 [SMILES: CC(C)Nc1ccc(-c2nc(-c3ccc4c(c3)NC(=O)CC4)no2)cc1C#N] | 435 | 2-(isopropylamino)-5-(3-(2-oxo- 1,2,3,4-tetrahydroquinolin-7-yl)- 1,2,4-oxadiazol-5-yl)benzonitrile |
| 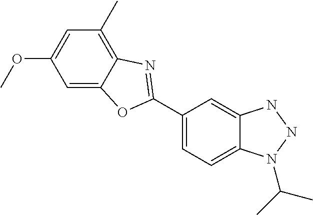 [SMILES: COc1cc(C)c2nc(-c3ccc4c(c3)NNN4C(C)C)oc2c1] | 436 | 2-(1-isopropyl-1H- benzo[d][1,2,3]triazol-5-yl)-6- methoxy-4- methylbenzo[d]oxazole |
| 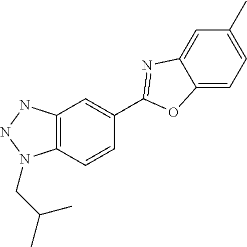 [SMILES: Cc1ccc2oc(-c3ccc4c(c3)NNN4CC(C)C)nc2c1] | 437 | 2-(1-isobutyl-1H- benzo[d][1,2,3]triazol-5-yl)-5- methylbenzo[d]oxazole |
| 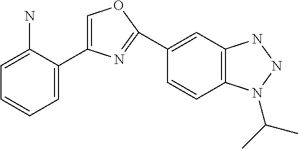 [SMILES: CC(C)N1NNc2cc(-c3nc(-c4ccccc4N)co3)ccc21] | 438 | 2-{2-[1-(propan-2-yl)-1H-1,2,3- benzotriazol-5-yl]-1,3-oxazol-4- yl}aniline |
| 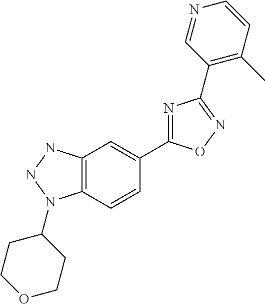 [SMILES: Cc1ccncc1-c1noc(-c2ccc3c(c2)NNN3C2CCOCC2)n1] | 439 | 5-[3-(4-methylpyridin-3-yl)-1,2,4- oxadiazol-5-yl]-1-(oxan-4-yl)- 1H-1,2,3-benzotriazole |
| 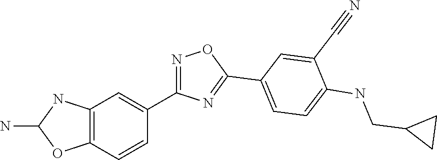 [SMILES: N#Cc1cc(-c2nc(-c3ccc4c(c3)NC(N)O4)no2)ccc1NCC1CC1] | 440 | 5-(3-(2-aminobenzo[d]thiazol-5- yl)-1,2,4-oxadiazol-5-yl)-2- ((cyclopropylmethyl)amino) benzonitrile |
| 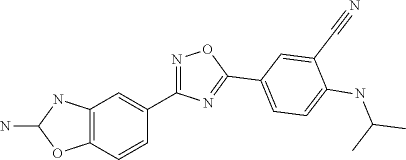 [SMILES: CC(C)Nc1ccc(-c2nc(-c3ccc4c(c3)NC(N)O4)no2)cc1C#N] | 441 | 5-(3-(2-aminobenzo[d]thiazol-5- yl)-1,2,4-oxadiazol-5-yl)-2- (isopropylamino)benzonitrile |
| 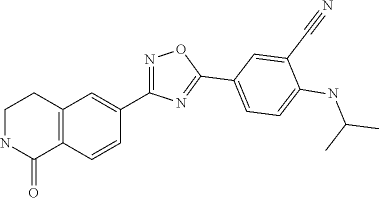 [SMILES: CC(C)Nc1ccc(-c2nc(-c3ccc4c(c3)CCNC4=O)no2)cc1C#N] | 442 | 2-(isopropylamino)-5-(3-(1-oxo- 1,2,3,4-tetrahydroisoquinolin-6- yl)-1,2,4-oxadiazol-5- yl)benzonitrile |
| 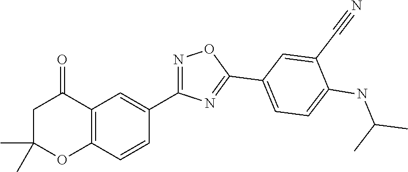 [SMILES: CC(C)Nc1ccc(-c2nc(-c3ccc4c(c3)C(=O)CC(C)(C)O4)no2)cc1C#N] | 443 | 5-(3-(2,2-diethyl-4-oxochroman- 6-yl)-1,2,4-oxadiazol-5-yl)-2- (isopropylamino)benzonitrile |
| 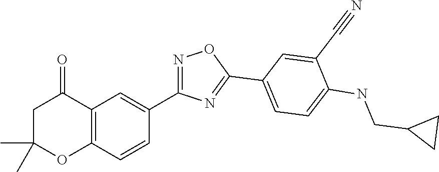 [SMILES: CC1(C)CC(=O)c2cc(-c3noc(-c4ccc(NCC5CC5)c(C#N)c4)n3)ccc2O1] | 444 | 2-((cyclopropylmethyl)amino)-5- (3-(2,2-diethyl-4-oxochroman-6- yl)-1,2,4-oxadiazol-5- yl)benzonitrile |
| 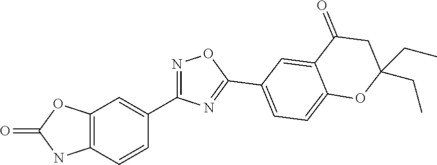 [SMILES: CCC1(CC)CC(=O)c2cc(-c3nc(-c4ccc5[nH]c(=O)oc5c4)no3)ccc2O1] | 445 | 6-(5-(2,2-diethyl-4-oxochroman- 6-yl)-1,2,4-oxadiazol-3- yl)benzo[d]oxazol-2(3H)-one |
| 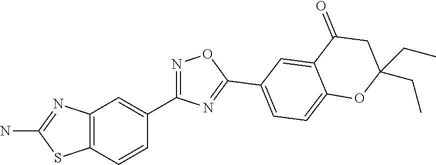 [SMILES: CCC1(CC)CC(=O)c2cc(-c3nc(-c4ccc5sc(N)nc5c4)no3)ccc2O1] | 446 | 6-(3-(2-aminobenzo[d]thiazol-5- yl)-1,2,4-oxadiazol-5-yl)-2,2- diethylchroman-4-one |
| 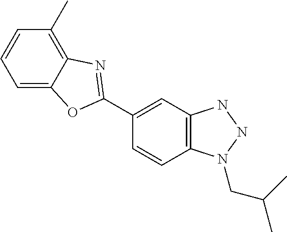 [SMILES: Cc1cccc2oc(-c3ccc4c(c3)NNN4CC(C)C)nc12] | 447 | 2-(1-isobutyl-1H- benzo[d][1,2,3]triazol-5-yl)-4- methylbenzo[d]oxazole |
| 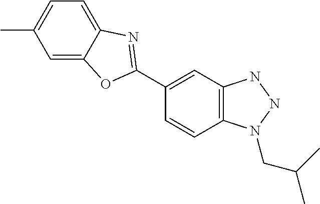 [SMILES: Cc1ccc2nc(-c3ccc4c(c3)NNN4CC(C)C)oc2c1] | 448 | 2-(1-isobutyl-1H- benzo[d][1,2,3]triazol-5-yl)-6- methylbenzo[d]oxazole |
| 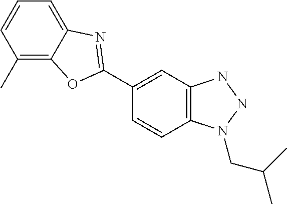 [SMILES: Cc1cccc2nc(-c3ccc4c(c3)NNN4CC(C)C)oc12] | 449 | 2-(1-isobutyl-1H- benzo[d][1,2,3]triazol-5-yl)-7- methylbenzo[d]oxazole |
| 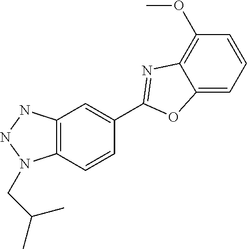 [SMILES: COc1cccc2oc(-c3ccc4c(c3)NNN4CC(C)C)nc12] | 450 | 2-(1-isobutyl-1H- benzo[d][1,2,3]triazol-5-yl)-4- methoxybenzo[d]oxazole |
| 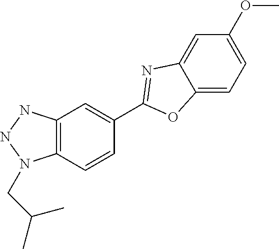 [SMILES: COc1ccc2oc(-c3ccc4c(c3)NNN4CC(C)C)nc2c1] | 451 | 2-(1-isobutyl-1H- benzo[d][1,2,3]triazol-5-yl)-5- methoxybenzo[d]oxazole |
| 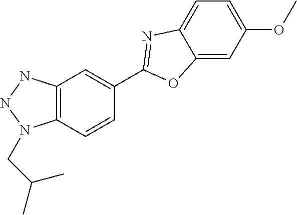 [SMILES: COc1ccc2nc(-c3ccc4c(c3)NNN4CC(C)C)oc2c1] | 452 | 2-(1-isobutyl-1H- benzo[d][1,2,3]triazol-5-yl)-6- methoxybenzo[d]oxazole |
| 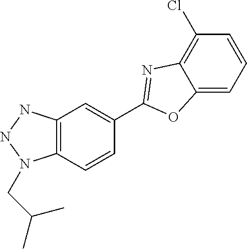 [SMILES: CC(C)CN1NNc2cc(-c3nc4c(Cl)cccc4o3)ccc21] | 453 | 4-chloro-2-(1-isobutyl-1H- benzo[d][1,2,3]triazol-5- yl)benzo[d]oxazole |
| 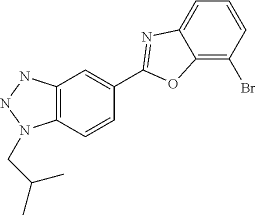 [SMILES: CC(C)CN1NNc2cc(-c3nc4cccc(Br)c4o3)ccc21] | 454 | 7-bromo-2-(1-isobutyl-1H- benzo[d][1,2,3]triazol-5- yl)benzo[d]oxazole |
| 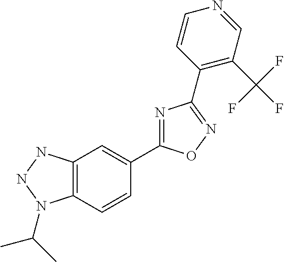 [SMILES: CC(C)N1NNc2cc(-c3nc(-c4ccncc4C(F)(F)F)no3)ccc21] | 455 | 1-(propan-2-yl)-5-{3-[3- (trifluoromethyl)pyridin-4-yl]- 1,2,4-oxadiazol-5-yl}-1H-1,2,3- benzotriazole |
| 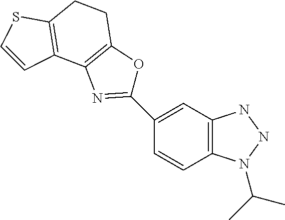 [SMILES: CC(C)N1NNc2cc(-c3nc4c(o3)CCc3sccc3-4)ccc21] | 456 | 4-[1-(propan-2-yl)-1H-1,2,3- benzotriazol-5-yl]-5-oxa-10-thia- 3-azatricyclo[7.3.0.0²,6]dodeca- 1(9),2(6),3,11-tetraene |
| 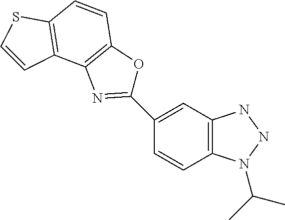 [SMILES: CC(C)N1NNc2cc(-c3nc4c(ccc5sccc54)o3)ccc21] | 457 | 4-[1-(propan-2-yl)-1H-1,2,3- benzotriazol-5-yl]-5-oxa-10-thia- 3-azatricyclo[7.3.0.0²,6]dodeca- 1(9),2(6),3,7,11-pentaene |
| 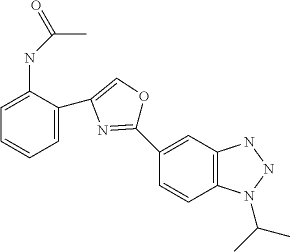 [SMILES: CC(=O)Nc1ccccc1-c1coc(-c2ccc3c(c2)NNN3C(C)C)n1] | 458 | N-(2-{2-[1-(propan-2-yl)-1H- 1,2,3-benzotriazol-5-yl]-1,3- oxazol-4-yl}phenyl)acetamide |
| 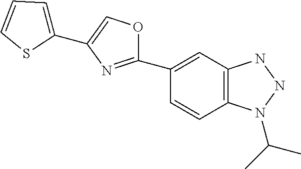 [SMILES: CC(C)N1NNc2cc(-c3nc(-c4cccs4)co3)ccc21] | 459 | 1-(propan-2-yl)-5-[4-(thiophen-2- yl)-1,3-oxazol-2-yl]-1H-1,2,3- benzotriazole |
| 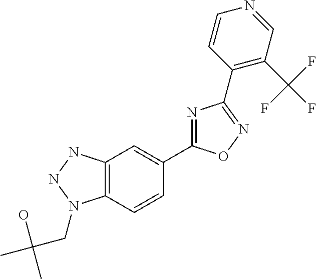 [SMILES: CC(C)(O)CN1NNc2cc(-c3nc(-c4ccncc4C(F)(F)F)no3)ccc21] | 460 | 2-methyl-1-(5-{3-[3- (trifluoromethyl)pyridin-4-yl]- 1,2,4-oxadiazol-5-yl}-1H-1,2,3- benzotriazol-1-yl)propan-2-ol |
| 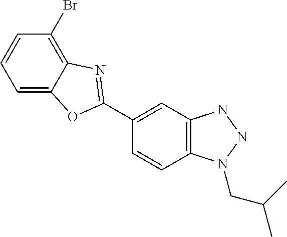 [SMILES: CC(C)CN1NNc2cc(-c3nc4c(Br)cccc4o3)ccc21] | 461 | 4-bromo-2-(1-isobutyl-1H- benzo[d][1,2,3]triazol-5- yl)benzo[d]oxazole |
| 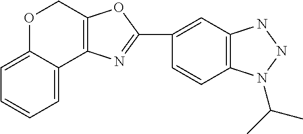 [SMILES: CC(C)N1NNc2cc(-c3nc4c(o3)COc3ccccc3-4)ccc21] | 462 | 5-{4H-chromeno[4,3- d][1,3]oxazol-2-yl}-1-(propan-2- yl)-1H-1,2,3-benzotriazole |
| 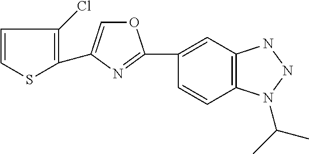 [SMILES: CC(C)N1NNc2cc(-c3nc(-c4sccc4Cl)co3)ccc21] | 463 | 5-[4-(3-chlorothiophen-2-yl)-1,3- oxazol-2-yl]-1-(propan-2-yl)-1H- 1,2,3-benzotriazole |
| 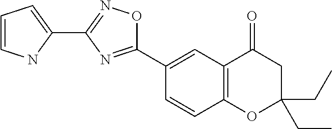 [SMILES: CCC1(CC)CC(=O)c2cc(-c3nc(-c4ccc[nH]4)no3)ccc2O1] | 464 | 6-(3-(1H-pyrrol-2-yl)-1,2,4- oxadiazol-5-yl)-2,2- diethylchroman-4-one |
| 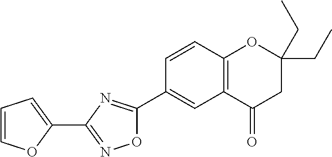 [SMILES: CCC1(CC)CC(=O)c2cc(-c3nc(-c4ccco4)no3)ccc2O1] | 465 | 2,2-diethyl-6-(3-(furan-2-yl)- 1,2,4-oxadiazol-5-yl)chroman-4- one |
| 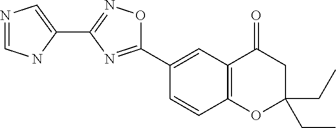 [SMILES: CCC1(CC)CC(=O)c2cc(-c3nc(-c4cnc[nH]4)no3)ccc2O1] | 466 | 6-(3-(1H-imidazol-5-yl)-1,2,4- oxadiazol-5-yl)-2,2- diethylchroman-4-one |
| 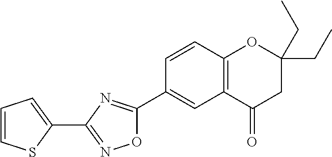 [SMILES: CCC1(CC)CC(=O)c2cc(-c3nc(-c4cccs4)no3)ccc2O1] | 467 | 2,2-diethyl-6-(3-(thiophen-2-yl)- 1,2,4-oxadiazol-5-yl)chroman-4- one |
| 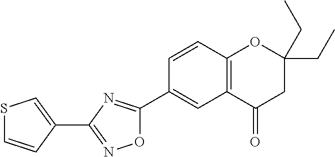 [SMILES: CCC1(CC)CC(=O)c2cc(-c3nc(-c4ccsc4)no3)ccc2O1] | 468 | 2,2-diethyl-6-(3-(thiophen-3-yl)- 1,2,4-oxadiazol-5-yl)chroman-4- one |
| 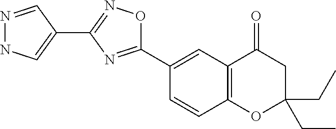 [SMILES: CCC1(CC)CC(=O)c2cc(-c3nc(-c4cn[nH]c4)no3)ccc2O1] | 469 | 6-(3-(1H-pyrazol-4-yl)-1,2,4- oxadiazol-5-yl)-2,2- diethylchroman-4-one |
| 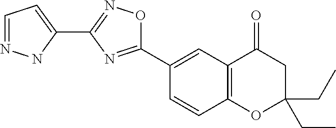 [SMILES: CCC1(CC)CC(=O)c2cc(-c3nc(-c4ccn[nH]4)no3)ccc2O1] | 470 | 6-(3-(1H-pyrazol-5-yl)-1,2,4- oxadiazol-5-yl)-2,2- diethylchroman-4-one |
| 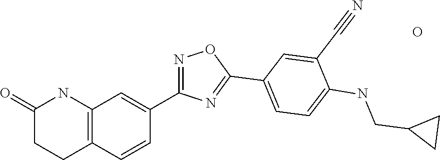 [SMILES: N#Cc1cc(-c2nc(-c3ccc4c(c3)NC(=O)CC4)no2)ccc1NCC1CC1 ; O] | 471 | 2-((cyclopropylmethyl)amino)-5- (3-(2-oxo-1,2,3,4- tetrahydroquinolin-7-yl)-1,2,4- oxadiazol-5-yl)benzonilrile |
| 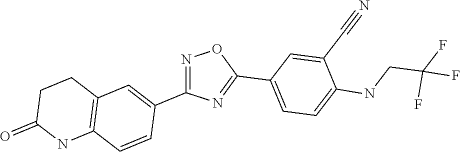 [SMILES: N#Cc1cc(-c2nc(-c3ccc4c(c3)CCC(=O)N4)no2)ccc1NCC(F)(F)F] | 472 | 5-[3-(2-oxo-1,2,3,4- tetrahydroquinolin-6-yl)-1,2,4- oxadiazol-5-yl]-2-[(2,2,2- trifluoroethyl)amino]benzonitrile |
| 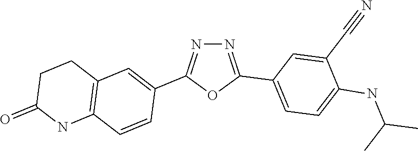 [SMILES: CC(C)Nc1ccc(-c2nnc(-c3ccc4c(c3)CCC(=O)N4)o2)cc1C#N] | 473 | 5-[5-(2-oxo-1,2,3,4- tetrahydroquinolin-6-yl)-1,3,4- oxadiazol-2-yl]-2-[(propan-2- yl)amino]benzonitrile |
| 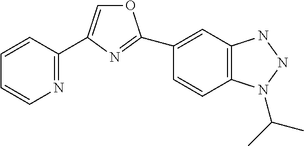 [SMILES: CC(C)N1NNc2cc(-c3nc(-c4ccccn4)co3)ccc21] | 474 | 1-(propan-2-yl)-5-[4-(pyridin-2- yl)-1,3-oxazol-2-yl]-1H-1,2,3- benzotriazole |
| 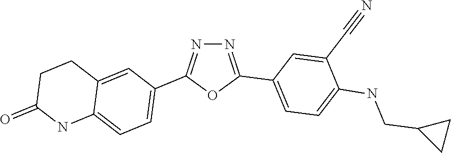 [SMILES: N#Cc1cc(-c2nnc(-c3ccc4c(c3)CCC(=O)N4)o2)ccc1NCC1CC1] | 475 | 2-[(cyclopropylmethyl)amino]-5- [5-(2-oxo-1,2,3,4- tetrahydroquinolin-6-yl)-1,3,4- oxadiazol-2-yl]benzonitrile |
| 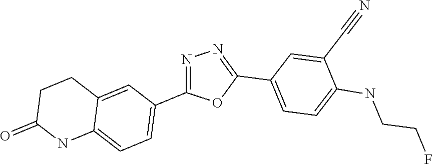 [SMILES: N#Cc1cc(-c2nnc(-c3ccc4c(c3)CCC(=O)N4)o2)ccc1NCCF] | 476 | 2-[(2-fluoroethyl)amino]-5-[5-(2- oxo-1,2,3,4-tetrahydroquinolin-6- yl)-1,3,4-oxadiazol-2- yl]benzonitrile |
| 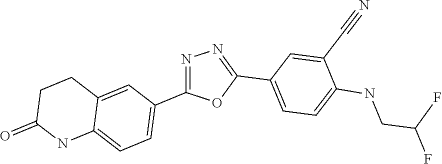 [SMILES: N#Cc1cc(-c2nnc(-c3ccc4c(c3)CCC(=O)N4)o2)ccc1NCC(F)F] | 477 | 2-[(2,2-difluoroethyl)amino]-5-[5- (2-oxo-1,2,3,4- tetrahydroquinolin-6-yl)-1,3,4- oxadiazol-2-yl]benzonitrile |
| 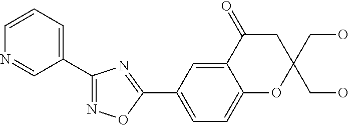 [SMILES: O=C1CC(CO)(CO)Oc2ccc(-c3nc(-c4cccnc4)no3)cc21] | 478 | 2,2-bis(hydroxymethyl)-6-[3- (pyridin-3-yl)-1,2,4-oxadiazol-5- yl]-3,4-dihydro-2H-1- benzopyran-4-one |
| 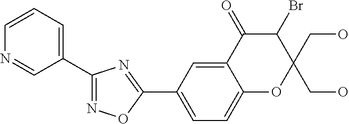 [SMILES: O=C1c2cc(-c3nc(-c4cccnc4)no3)ccc2OC(CO)(CO)C1Br] | 479 | 3-bromo-2,2-bis(hydroxymethyl)- 6-[3-(pyridin-3-yl)-1,2,4- oxadiazol-5-yl]-3,4-dihydro-2H- 1-benzopyran-4-one |
| 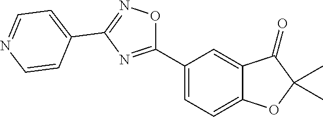 [SMILES: CC1(C)Oc2ccc(-c3nc(-c4ccncc4)no3)cc2C1=O] | 480 | 2,2-dimethyl-5-[3-(pyridin-4-yl)- 1,2,4-oxadiazol-5-yl]-2,3- dihydro-1-benzofuran-3-one |
| 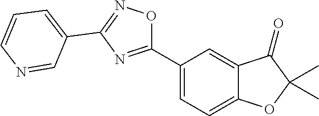 [SMILES: CC1(C)Oc2ccc(-c3nc(-c4cccnc4)no3)cc2C1=O] | 481 | 2,2-dimethyl-5-[3-(pyridin-3-yl)- 1,2,4-oxadiazol-5-yl]-2,3- dihydro-1-benzofuran-3-one |
| 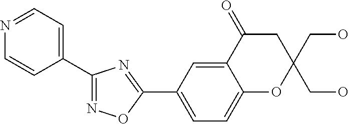 [SMILES: O=C1CC(CO)(CO)Oc2ccc(-c3nc(-c4ccncc4)no3)cc21] | 482 | 2,2-bis(hydroxymethyl)-6-[3- (pyridin-4-yl)-1,2,4-oxadiazol-5- yl]-3,4-dihydro-2H-1- benzopyran-4-one |
| 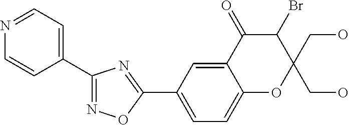 [SMILES: O=C1c2cc(-c3nc(-c4ccncc4)no3)ccc2OC(CO)(CO)C1Br] | 483 | 3-bromo-2,2-bis(hydroxymethyl)- 6-[3-(pyridin-4-yl)-1,2,4- oxadiazol-5-yl]-3,4-dihydro-2H- 1-benzopyran-4-one |
| 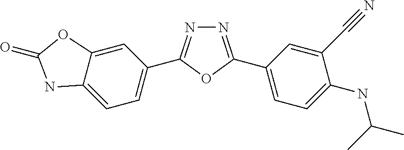 [SMILES: CC(C)Nc1ccc(-c2nnc(-c3ccc4[nH]c(=O)oc4c3)o2)cc1C#N] | 484 | 5-[5-(2-oxo-2,3-dihydro-1,3- benzoxazol-6-yl)-1,3,4- oxadiazol-2-yl]-2-[(propan-2- yl)amino]benzonitrile |
| 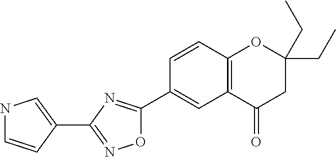 [SMILES: CCC1(CC)CC(=O)c2cc(-c3nc(-c4cc[nH]c4)no3)ccc2O1] | 485 | 6-(3-(1H-pyrrol-3-yl)-1,2,4- oxadiazol-5-yl)-2,2- diethylchroman-4-one |
| 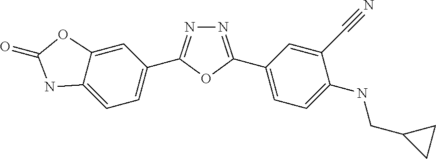 [SMILES: N#Cc1cc(-c2nnc(-c3ccc4[nH]c(=O)oc4c3)o2)ccc1NCC1CC1] | 486 | 2-[(cyclopropylmethyl)amino]-5- [5-(2-oxo-2,3-dihydro-1,3- benzoxazol-6-yl)-1,3,4-oxadiazol- 2-yl]benzonitrile |
| 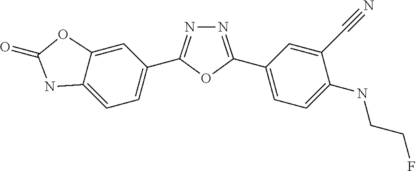 [SMILES: N#Cc1cc(-c2nnc(-c3ccc4[nH]c(=O)oc4c3)o2)ccc1NCCF] | 487 | 2-[(2-fluoroethyl)amino]-5-[5-(2- oxo-2,3-dihydro-1,3-benzoxazol- 6-yl)-1,3,4-oxadiazol-2- yl]benzonitrile |
| 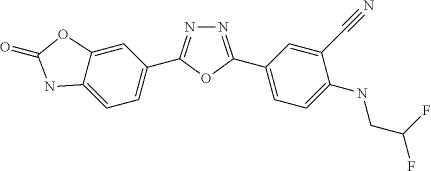 [SMILES: N#Cc1cc(-c2nnc(-c3ccc4[nH]c(=O)oc4c3)o2)ccc1NCC(F)F] | 488 | 2-[(2,2-difluoroethyl)amino]-5-[5- (2-oxo-2,3-dihydro-1,3- benzoxazol-6-yl)-1,3,4-oxadiazol- 2-yl]benzonitrile |
| 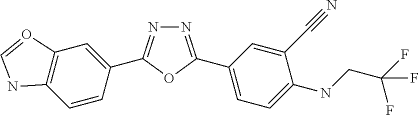 | 489 | 5-[3-(1H-1,3-benzodiazol-5-yl)- 1,2,4-oxadiazol-5-yl]-2-[(2,2,2- trifluoroethyl)amino]benzonitrile |
| 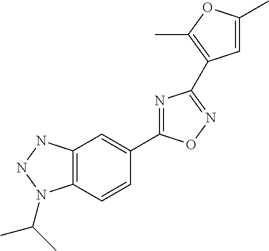 [SMILES: Cc1cc(-c2noc(-c3ccc4c(c3)NNN4C(C)C)n2)c(C)o1] | 490 | 5-[3-(2,5-dimethylfuran-3-yl)- 1,2,4-oxadiazol-5-yl]-1-(propan- 2-yl)-1H-1,2,3-benzotriazole |
| 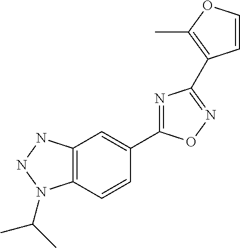 [SMILES: Cc1occc1-c1noc(-c2ccc3c(c2)NNN3C(C)C)n1] | 491 | 5-[3-(2-methylfuran-3-yl)-1,2,4- oxadiazol-5-yl]-1-(propan-2-yl)- 1H-1,2,3-benzotriazole |
| 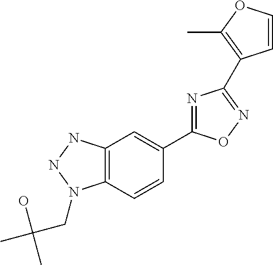 [SMILES: Cc1occc1-c1noc(-c2ccc3c(c2)NNN3CC(C)(C)O)n1] | 492 | 2-methyl-1-{5-[3-(2-methylfuran- 3-yl)-1,2,4-oxadiazol-5-yl]-1H- 1,2,3-benzotriazol-1-yl}propan-2- ol |
| 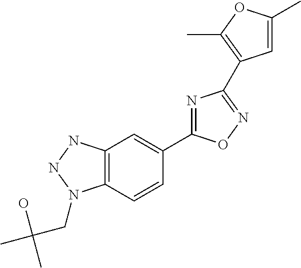 [SMILES: Cc1cc(-c2noc(-c3ccc4c(c3)NNN4CC(C)(C)O)n2)c(C)o1] | 493 | 1-{5-[3-(2,5-dimethylfuran-3-yl)- 1,2,4-oxadiazol-5-yl]-1H-1,2,3- benzotriazol-1-yl}-2- methylpropan-2-ol |
| 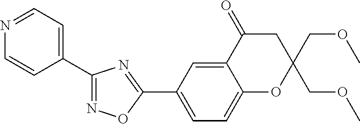 [SMILES: COCC1(COC)CC(=O)c2cc(-c3nc(-c4ccncc4)no3)ccc2O1] | 494 | 2,2-bis(methoxymethyl)-6-[3- (pyridin-4-yl)-1,2,4-oxadiazol-5- yl]-3,4-dihydro-2H-1- benzopyran-4-one |
| 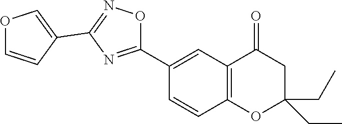 [SMILES: CCC1(CC)CC(=O)c2cc(-c3nc(-c4ccoc4)no3)ccc2O1] | 495 | 2,2-diethyl-6-(3-(furan-3-yl)- 1,2,4-oxadiazol-5-yl)chroman-4- one |
| 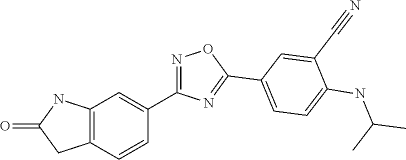 [SMILES: CC(C)Nc1ccc(-c2nc(-c3ccc4c(c3)NC(=O)C4)no2)cc1C#N] | 496 | 2-(isopropylamino)-5-(3-(2- oxoindolin-6-yl)-1,2,4-oxadiazol- 5-yl)benzonitrile |
| 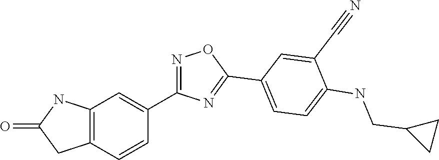 [SMILES: N#Cc1cc(-c2nc(-c3ccc4c(c3)NC(=O)C4)no2)ccc1NCC1CC1] | 497 | 2-((cyclopropylmethyl)amino)-5- (3-(2-oxoindolin-6-yl)-1,2,4- oxadiazol-5-yl)benzonitrile |
| 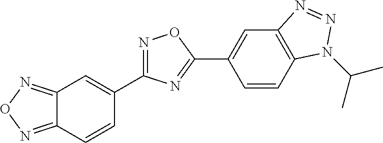 [SMILES: CC(C)n1nnc2cc(-c3nc(-c4ccc5nonc5c4)no3)ccc21] | 498 | 5-{5-[1-(propan-2-yl)-1H-1,2,3- benzotriazol-5-yl]-1,2,4- oxadiazol-3-yl}-2,1,3- benzoxadiazole |
| 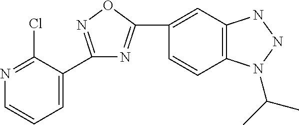 [SMILES: CC(C)N1NNc2cc(-c3nc(-c4cccnc4Cl)no3)ccc21] | 499 | 5-[3-(2-chloropyridin-3-yl)-1,2,4- oxadiazol-5-yl]-1-(propan-2-yl)- 1H-1,2,3-benzotriazole |
| 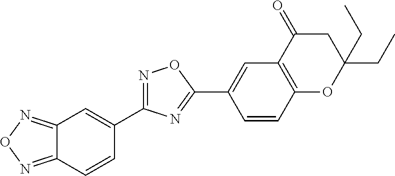 [SMILES: CCC1(CC)CC(=O)c2cc(-c3nc(-c4ccc5nonc5c4)no3)ccc2O1] | 500 | 6-[3-(2,1,3-benzoxadiazol-5-yl)- 1,2,4-oxadiazol-5-yl]-2,2-diethyl- 3,4-dihydro-2H-1-benzopyran-4- one |
| 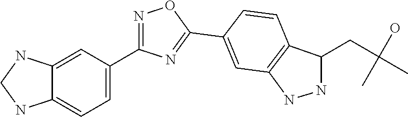 [SMILES: CC(C)(O)CC1NNc2cc(-c3nc(-c4ccc5c(c4)NCN5)no3)ccc21] | 501 | 1-{5-[3-(1H-1,3-benzodiazol-6- yl)-1,2,4-oxadiazol-5-yl]-1H- 1,2,3-benzotriazol-1-yl}-2- methylpropan-2-ol |
| 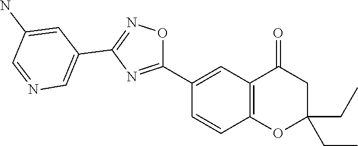 [SMILES: CCC1(CC)CC(=O)c2cc(-c3nc(-c4cncc(N)c4)no3)ccc2O1] | 502 | 6-(3-(5-aminopyridin-3-yl)-1,2,4- oxadiazol-5-yl)-2,2- diethylchroman-4-one |
| 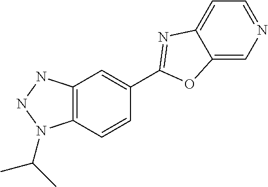 [SMILES: CC(C)N1NNc2cc(-c3nc4ccncc4o3)ccc21] | 503 | 2-(1-isopropyl-1H- benzo[d][1,2,3]triazol-5- yl)oxazolo[5,4-c]pyridine |
| 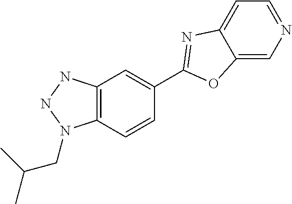 [SMILES: CC(C)CN1NNc2cc(-c3nc4ccncc4o3)ccc21] | 504 | 2-(1-isobutyl-1H- benzo[d][1,2,3]triazol-5- yl)oxazolo[5,4-c]pyridine |
| 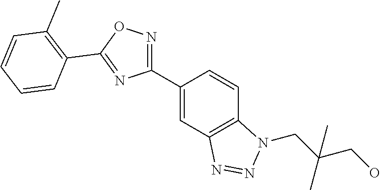 [SMILES: Cc1ccccc1-c1nc(-c2ccc3c(c2)nnn3CC(C)(C)CO)no1] | 505 | 2,2-dimethyl-3-(5-(5-(o-tolyl)- 1,2,4-oxadiazol-3-yl)-1H- benzo[d][1,2,3]triazol-1- yl)propan-1-ol |
| 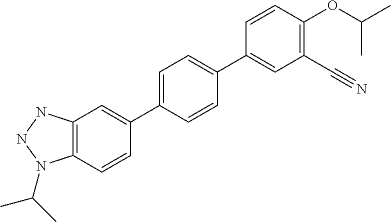 [SMILES: CC(C)Oc1ccc(-c2ccc(-c3ccc4c(c3)NNN4C(C)C)cc2)cc1C#N] | 506 | 4-isopropoxy-4′-(1-isopropyl-1H- benzo[d][1,2,3]triazol-5-yl)-[1,1′- biphenyl]-3-carbonitrile |
| 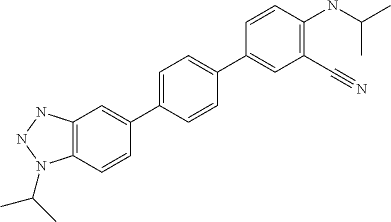 [SMILES: CC(C)Nc1ccc(-c2ccc(-c3ccc4c(c3)NNN4C(C)C)cc2)cc1C#N] | 507 | 4′-(1-isopropyl-1H- benzo[d][1,2,3]triazol-5-yl)-4- (isopropylamino)-[1,1′-biphenyl]- 3-carbonitrile |
| 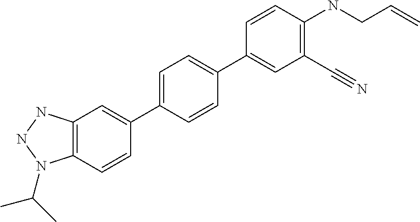 [SMILES: C=CCNc1ccc(-c2ccc(-c3ccc4c(c3)NNN4C(C)C)cc2)cc1C#N] | 508 | 4-(allylamino)-4′-(1-isopropyl- 1H-benzo[d][1,2,3]triazol-5-yl)- [1,1′-biphenyl]-3-carbonitrile |
| 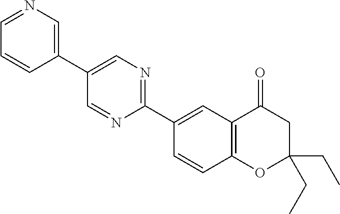 [SMILES: CCC1(CC)CC(=O)c2cc(-c3ncc(-c4cccnc4)cn3)ccc2O1] | 509 | 2,2-diethyl-6-(5-(pyridin-3- yl)pyrimidin-2-yl)chroman-4-one |
| 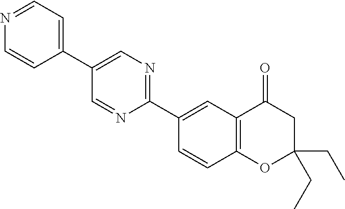 [SMILES: CCC1(CC)CC(=O)c2cc(-c3ncc(-c4ccncc4)cn3)ccc2O1] | 510 | 2,2-diethyl-6-(5-(pyridin-4- yl)pyrimidin-2-yl)chroman-4-one |
| 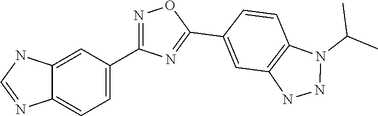 [SMILES: CC(C)N1NNc2cc(-c3nc(-c4ccc5nc[nH]c5c4)no3)ccc21] | 511 | 5-[3-(1H-1,3-benzodiazol-6-yl)- 1,2,4-oxadiazol-5-yl]-1-(propan- 2-yl)-1H-1,2,3-benzotriazole |
| 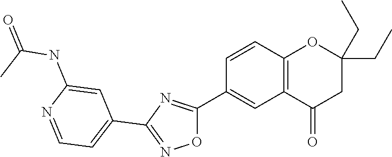 [SMILES: CCC1(CC)CC(=O)c2cc(-c3nc(-c4ccnc(NC(C)=O)c4)no3)ccc2O1] | 512 | N-(4-(5-(2,2-diethyl-4- oxochroman-6-yl)-1,2,4- oxadiazol-3-yl)pyridin-2- yl)acetamide |
| 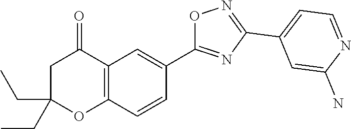 [SMILES: CCC1(CC)CC(=O)c2cc(-c3nc(-c4ccnc(N)c4)no3)ccc2O1] | 513 | 6-(3-(2-aminopyridin-4-yl)-1,2,4- oxadiazol-5-yl)-2,2- diethylchroman-4-one |
| 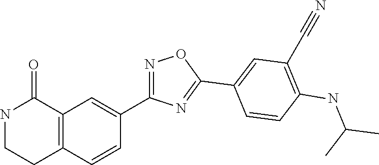 [SMILES: CC(C)Nc1ccc(-c2nc(-c3ccc4c(c3)C(=O)NCC4)no2)cc1C#N] | 514 | 2-(isopropylamino)-5-(3-(1-oxo- 1,2,3,4-tetrahydroisoquinolin-7- yl)-1,2,4-oxadiazol-5- yl)benzonitrile |
| 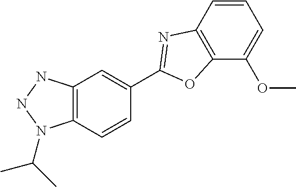 [SMILES: COc1cccc2nc(-c3ccc4c(c3)NNN4C(C)C)oc12] | 515 | 2-(1-isopropyl-1H- benzo[d][1,2,3]triazol-5-yl)-7- methoxybenzo[d]oxazole |
| 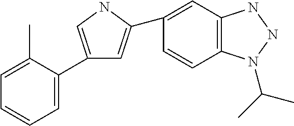 [SMILES: Cc1ccccc1-c1c[nH]c(-c2ccc3c(c2)NNN3C(C)C)c1] | 516 | 5-[4-(2-methylphenyl)-1H-pyrrol- 2-yl]-1-(propan-2-yl)-1H-1,2,3- benzotriazole |
| 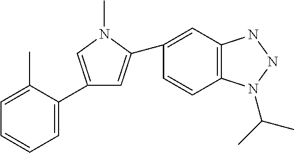 [SMILES: Cc1ccccc1-c1cc(-c2ccc3c(c2)NNN3C(C)C)n(C)c1] | 517 | 5-[1-methyl-4-(2-methylphenyl)- 1H-pyrrol-2-yl]-1-(propan-2-yl)- 1H-1,2,3-benzotriazole |
| 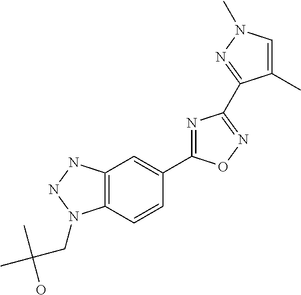 [SMILES: Cc1cn(C)nc1-c1noc(-c2ccc3c(c2)NNN3CC(C)(C)O)n1] | 518 | 1-{5-[3-(1,4-dimethyl-1H- pyrazol-3-yl)-1,2,4-oxadiazol-5- yl]-1H-1,2,3-benzotriazol-1-yl}- 2-methylpropan-2-ol |
| 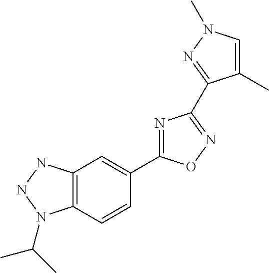 [SMILES: Cc1cn(C)nc1-c1noc(-c2ccc3c(c2)NNN3C(C)C)n1] | 519 | 5-[3-(1,4-dimethyl-1H-pyrazol-3- yl)-1,2,4-oxadiazol-5-yl]-1- (propan-2-yl)-1H-1,2,3- benzotriazole |
| 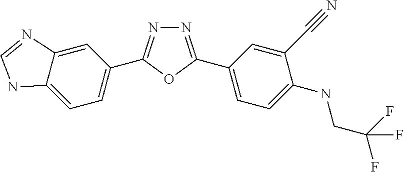 [SMILES: N#Cc1cc(-c2nnc(-c3ccc4[nH]cnc4c3)o2)ccc1NCC(F)(F)F] | 520 | 5-[5-(1H-1,3-benzodiazol-5-yl)- 1,3,4-oxadiazol-2-yl]-2-[(2,2- difluoropropyl)amino]benzonitrile |
| 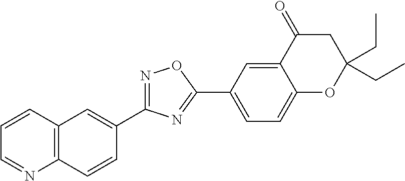 [SMILES: CCC1(CC)CC(=O)c2cc(-c3nc(-c4ccc5ncccc5c4)no3)ccc2O1] | 521 | 2,2-diethyl-6-(3-(quinolin-6-yl)- 1,2,4-oxadiazol-5-yl)chroman-4- one |
| 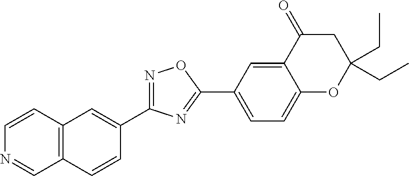 [SMILES: CCC1(CC)CC(=O)c2cc(-c3nc(-c4ccc5cnccc5c4)no3)ccc2O1] | 522 | 2,2-diethyl-6-(3-(isoquinolin-6- yl)-1,2,4-oxadiazol-5-yl)chroman- 4-one |
| 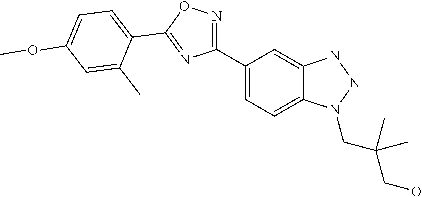 [SMILES: COc1ccc(-c2nc(-c3ccc4c(c3)NNN4CC(C)(C)CO)no2)c(C)c1] | 523 | 3-(5-(5-(4-methoxy-2- methylphenyl)-1,2,4-oxadiazol-3- yl)-1H-benzo[d][1,2,3]triazol-1- yl)-2,2-dimethylpropan-1-ol |
| 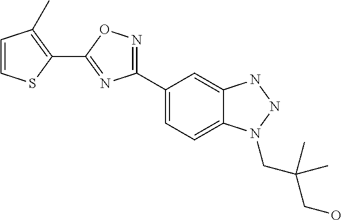 [SMILES: Cc1ccsc1-c1nc(-c2ccc3c(c2)NNN3CC(C)(C)CO)no1] | 524 | 2,2-dimethyl-3-(5-(5-(3- methylthiophen-2-yl)-1,2,4- oxadiazol-3-yl)-1H- benzo[d][1,2,3]triazol-1- yl)propan-1-ol |
| 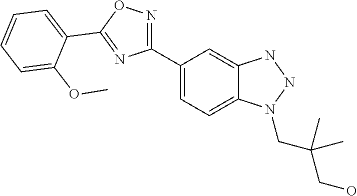 [SMILES: COc1ccccc1-c1nc(-c2ccc3c(c2)NNN3CC(C)(C)CO)no1] | 525 | 3-(5-(5-(2-methoxyphenyl)-1,2,4- oxadiazol-3-yl)-1H- benzo[d][1,2,3]triazol-1-yl)-2,2- dimethylpropan-1-ol |
| 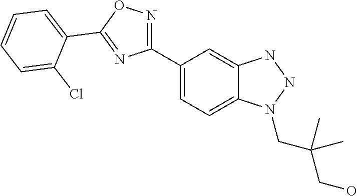 [SMILES: CC(C)(CO)CN1NNc2cc(-c3noc(-c4ccccc4Cl)n3)ccc21] | 526 | 3-(5-(5-(2-chlorophenyl)-1,2,4- oxadiazol-3-yl)-1H- benzo[d][1,2,3]triazol-1-yl)-2,2- dimethylpropan-1-ol |
| 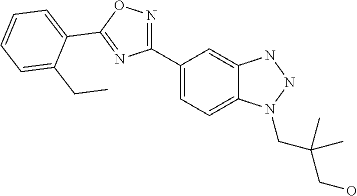 [SMILES: CCc1ccccc1-c1nc(-c2ccc3c(c2)NNN3CC(C)(C)CO)no1] | 527 | 3-(5-(5-(2-ethylphenyl)-1,2,4- oxadiazol-3-yl)-1H- benzo[d][1,2,3]triazol-1-yl)-2,2- dimethylpropan-1-ol |
| 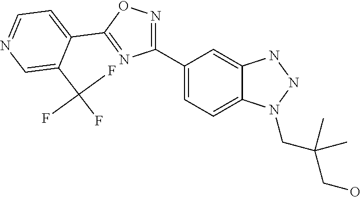 [SMILES: CC(C)(CO)CN1NNc2cc(-c3noc(-c4ccncc4C(F)(F)F)n3)ccc21] | 528 | 2,2-dimethyl-3-(5-(5-(3- (trifluoromethyl)pyridin-4-yl)- 1,2,4-oxadiazol-3-yl)-1H- benzo[d][1,2,3]triazol-1- yl)propan-1-ol |
| 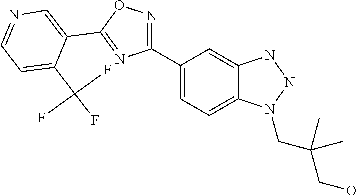 [SMILES: CC(C)(CO)CN1NNc2cc(-c3noc(-c4cnccc4C(F)(F)F)n3)ccc21] | 529 | 2,2-dimethyl-3-(5-(5-(4- (trifluoromethyl)pyridin-3-yl)- 1,2,4-oxadiazol-3-yl)-1H- benzo[d][1,2,3]triazol-1- yl)propan-1-ol |
| 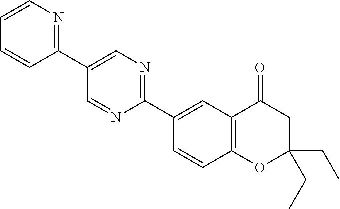 [SMILES: CCC1(CC)CC(=O)c2cc(-c3ncc(-c4ccccn4)cn3)ccc2O1] | 530 | 2,2-diethyl-6-(5-(pyridin-2- yl)pyrimidin-2-yl)chroman-4-one |
| 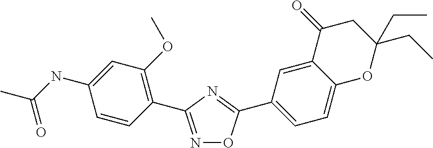 [SMILES: CCC1(CC)CC(=O)c2cc(-c3nc(-c4ccc(NC(C)=O)cc4OC)no3)ccc2O1] | 531 | N-{4-[5-(2,2-diethyl-4-oxo-3,4- dihydro-2H-1-benzopyran-6-yl)- 1,2,4-oxadiazol-3-yl]-3- methoxyphenyl}acetamide |
| 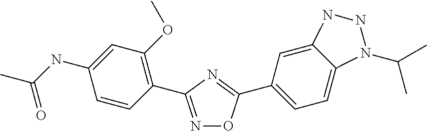 [SMILES: COc1cc(NC(C)=O)ccc1-c1noc(-c2ccc3c(c2)NNN3C(C)C)n1] | 532 | N-(3-methoxy-4-{5-[1-(propan-2- yl)-1H-1,2,3-benzotriazol-5-yl]- 1,2,4-oxadiazol-3- yl}phenyl)acetamide |
| 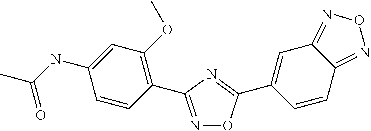 [SMILES: COc1cc(NC(C)=O)ccc1-c1noc(-c2ccc3nonc3c2)n1] | 533 | N-{4-[5-(2,1,3-benzoxadiazol-5- yl)-1,2,4-oxadiazol-3-yl]-3- methoxyphenyl}acetamide |
| 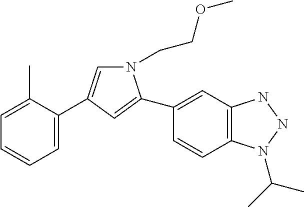 [SMILES: COCCn1cc(-c2ccccc2C)cc1-c1ccc2c(c1)NNN2C(C)C] | 534 | 5-[1-(2-methoxyethyl)-4-(2- methylphenyl)-1H-pyrrol-2-yl]- 1-(propan-2-yl)-1H-1,2,3- benzotriazole |
| 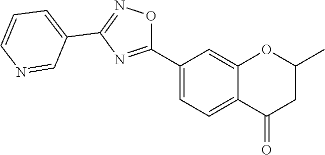 [SMILES: CC1CC(=O)c2ccc(-c3nc(-c4cccnc4)no3)cc2O1] | 535 | 2-methyl-7-[3-(pyridin-3-yl)- 1,2,4-oxadiazol-5-yl]-3,4- dihydro-2H-1-benzopyran-4-one |
| 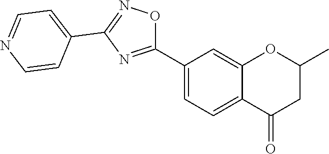 [SMILES: CC1CC(=O)c2ccc(-c3nc(-c4ccncc4)no3)cc2O1] | 536 | 2-methyl-7-[3-(pyridin-4-yl)- 1,2,4-oxadiazol-5-yl]-3,4- dihydro-2H-1-benzopyran-4-one |
| 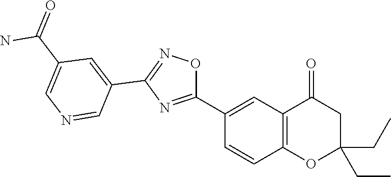 [SMILES: CCC1(CC)CC(=O)c2cc(-c3nc(-c4cncc(C(N)=O)c4)no3)ccc2O1] | 537 | 5-(5-(2,2-diethyl-4-oxochroman- 6-yl)-1,2,4-oxadiazol-3- yl)nicotinamide |
| 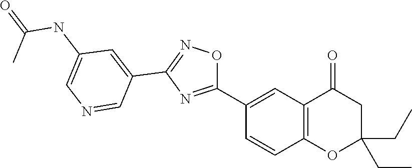 [SMILES: CCC1(CC)CC(=O)c2cc(-c3nc(-c4cncc(NC(C)=O)c4)no3)ccc2O1] | 538 | N-(5-(5-(2,2-diethyl-4- oxochroman-6-yl)-1,2,4- oxadiazol-3-yl)pyridin-3- yl)acetamide |
| 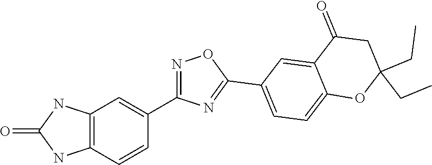 [SMILES: CCC1(CC)CC(=O)c2cc(-c3nc(-c4ccc5[nH]c(=O)[nH]c5c4)no3)ccc2O1] | 539 | 5-(5-(2,2-diethyl-4-oxochroman- 6-yl)-1,2,4-oxadiazol-3-yl)-1H- benzo[d]imidazol-2(3H)-one |
| 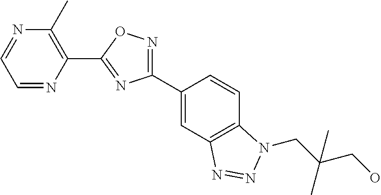 [SMILES: Cc1nccnc1-c1nc(-c2ccc3c(c2)nnn3CC(C)(C)CO)no1] | 540 | 2,2-dimethyl-3-(5-(5-(3- methylpyrazin-2-yl)-1,2,4- oxadiazol-3-yl)-1H- benzo[d][1,2,3]triazol-1- yl)propan-1-ol |
| 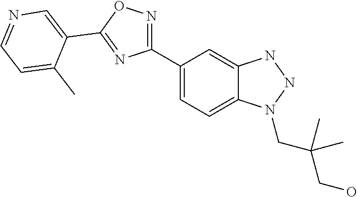 [SMILES: Cc1ccncc1-c1nc(-c2ccc3c(c2)NNN3CC(C)(C)CO)no1] | 541 | 2,2-dimethyl-3-(5-(5-(4- methylpyridin-3-yl)-1,2,4- oxadiazol-3-yl)-1H- benzo[d][1,2,3]triazol-1- yl)propan-1-ol |
| 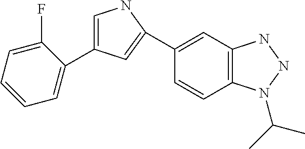 [SMILES: CC(C)N1NNc2cc(-c3cc(-c4ccccc4F)c[nH]3)ccc21] | 542 | 5-[4-(2-fluorophenyl)-1H-pyrrol- 2-yl]-1-(propan-2-yl)-1H-1,2,3- benzotriazole |
| 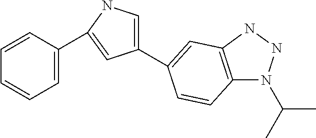 [SMILES: CC(C)N1NNc2cc(-c3c[nH]c(-c4ccccc4)c3)ccc21] | 543 | 5-(5-phenyl-1H-pyrrol-3-yl)-1- (propan-2-yl)-1H-1,2,3- benzotriazole |
| 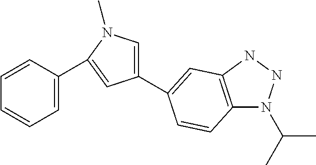 [SMILES: CC(C)N1NNc2cc(-c3cc(-c4ccccc4)n(C)c3)ccc21] | 544 | 5-(1-methyl-5-phenyl-1H-pyrrol- 3-yl)-1-(propan-2-yl)-1H-1,2,3- benzotriazole |
| 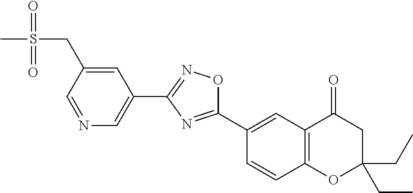 [SMILES: CCC1(CC)CC(=O)c2cc(-c3nc(-c4cncc(CS(C)(=O)=O)c4)no3)ccc2O1] | 545 | N-(5-(5-(2,2-diethyl-4- oxochroman-6-yl)-1,2,4- oxadiazol-3-yl)pyridin-3- yl)methanesulfonamide |
| 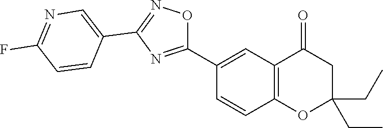 [SMILES: CCC1(CC)CC(=O)c2cc(-c3nc(-c4ccc(F)nc4)no3)ccc2O1] | 546 | 2,2-diethyl-6-(3-(6-fluoropyridin- 3-yl)-1,2,4-oxadiazol-5- yl)chroman-4-one |
| 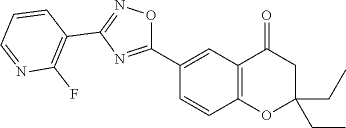 [SMILES: CCC1(CC)CC(=O)c2cc(-c3nc(-c4cccnc4F)no3)ccc2O1] | 547 | 2,2-diethyl-6-(3-(2-fluoropyridin- 3-yl)-1,2,4-oxadiazol-5- yl)chroman-4-one |
| 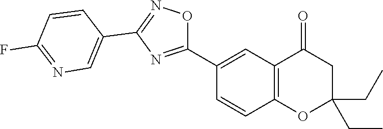 [SMILES: CCC1(CC)CC(=O)c2cc(-c3nc(-c4ccc(F)nc4)no3)ccc2O1] | 548 | 2,2-diethyl-6-(3-(6- (trifluoromethyl)pyridin-3-yl)- 1,2,4-oxadiazol-5-yl)chroman-4- one |
| 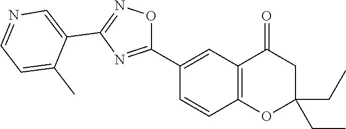 [SMILES: CCC1(CC)CC(=O)c2cc(-c3nc(-c4cnccc4C)no3)ccc2O1] | 549 | 2,2-diethyl-6-(3-(4- methylpyridin-3-yl)-1,2,4- oxadiazol-5-yl)chroman-4-one |
| 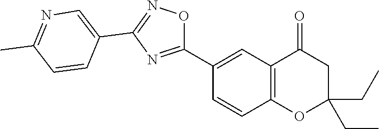 [SMILES: CCC1(CC)CC(=O)c2cc(-c3nc(-c4ccc(C)nc4)no3)ccc2O1] | 550 | 2,2-diethyl-6-(3-(6- methylpyridin-3-yl)-1,2,4- oxadiazol-5-yl)chroman-4-one |
| 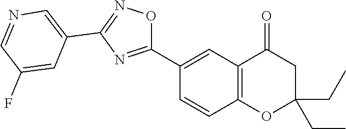 [SMILES: CCC1(CC)CC(=O)c2cc(-c3nc(-c4cncc(F)c4)no3)ccc2O1] | 551 | 2,2-diethyl-6-(3-(5-fluoropyridin- 3-yl)-1,2,4-oxadiazol-5- yl)chroman-4-one |
| 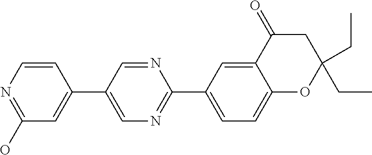 [SMILES: CCC1(CC)CC(=O)c2cc(-c3ncc(-c4ccnc(O)c4)cn3)ccc2O1] | 552 | 2,2-diethyl-6-(5-(2- hydroxypyridin-4-yl)pyrimidin-2- yl)chroman-4-one |
| 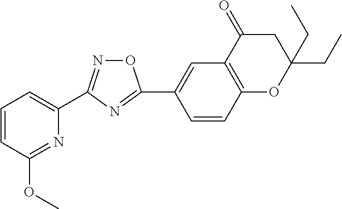 [SMILES: CCC1(CC)CC(=O)c2cc(-c3nc(-c4cccc(OC)n4)no3)ccc2O1] | 553 | 2,2-diethyl-6-[3-(6- methoxypyridin-2-yl)-1,2,4- oxadiazol-5-yl]-3,4-dihydro-2H- 1-benzopyran-4-one |
| 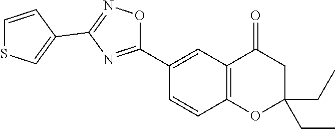 [SMILES: CCC1(CC)CC(=O)c2cc(-c3nc(-c4ccsc4)no3)ccc2O1] | 554 | 2,2-diethyl-6-(3-(thiophen-3-yl)- 1,2,4-oxadiazol-5-yl)-2,3- dihydroquinolin-4(1H)-one |
| 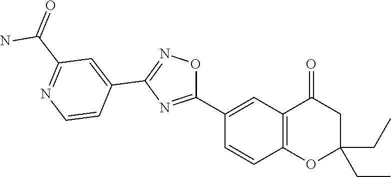 [SMILES: CCC1(CC)CC(=O)c2cc(-c3nc(-c4ccnc(C(N)=O)c4)no3)ccc2O1] | 555 | 4-(5-(2,2-diethyl-4-oxochroman- 6-yl)-1,2,4-oxadiazol-3- yl)picolinamide |
| 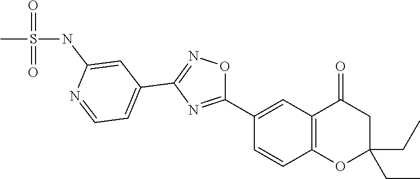 [SMILES: CCC1(CC)CC(=O)c2cc(-c3nc(-c4ccnc(NS(C)(=O)=O)c4)no3)ccc2O1] | 556 | N-(4-(5-(2,2-diethyl-4- oxochroman-6-yl)-1,2,4- oxadiazol-3-yl)pyridin-2- yl)methanesulfonamide |
| 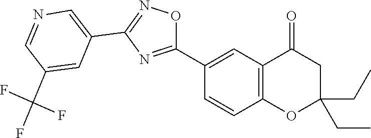 [SMILES: CCC1(CC)CC(=O)c2cc(-c3nc(-c4cncc(C(F)(F)F)c4)no3)ccc2O1] | 557 | 2,2-diethyl-6-(3-(5- (trifluoromethyl)pyridin-3-yl)- 1,2,4-oxadiazol-5-yl)chroman-4- one |
| 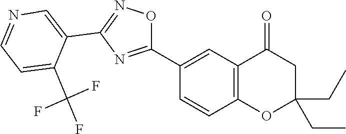 [SMILES: CCC1(CC)CC(=O)c2cc(-c3nc(-c4cnccc4C(F)(F)F)no3)ccc2O1] | 558 | 2,2-diethyl-6-(3-(4- (trifluoromethyl)pyridin-3-yl)- 1,2,4-oxadiazol-5-yl)chroman-4- one |
| 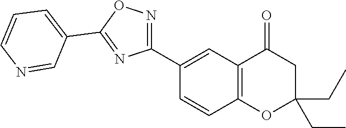 [SMILES: CCC1(CC)CC(=O)c2cc(-c3noc(-c4cccnc4)n3)ccc2O1] | 559 | 2,2-diethyl-6-(5-(pyridin-3-yl)- 1,2,4-oxadiazol-3-yl)chroman-4- one |
| 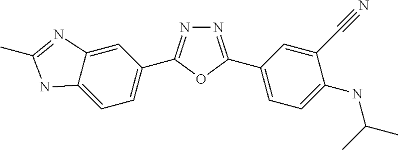 [SMILES: Cc1nc2cc(-c3nnc(-c4ccc(NC(C)C)c(C#N)c4)o3)ccc2[nH]1] | 560 | 5-[5-(2-methyl-1H-1,3- benzodiazol-5-yl)-1,3,4- oxadiazol-2-yl]-2-[(propan-2- yl)amino]benzonitrile |
| 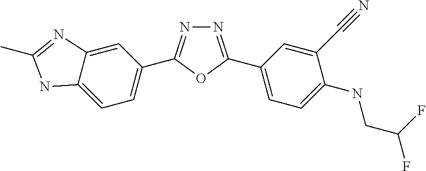 [SMILES: Cc1nc2cc(-c3nnc(-c4ccc(NCC(F)F)c(C#N)c4)o3)ccc2[nH]1] | 561 | 2-[(2,2-difluoroethyl)amino]-5-[5- (2-methyl-1H-1,3-benzodiazol-5- yl)-1,3,4-oxadiazol-2- yl]benzonitrile |
| 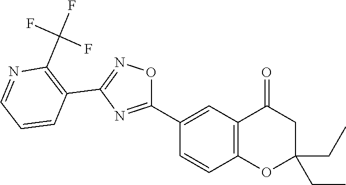 [SMILES: CCC1(CC)CC(=O)c2cc(-c3nc(-c4cccnc4C(F)(F)F)no3)ccc2O1] | 562 | 2,2-diethyl-6-(3-(2- (trifluoromethyl)pyridin-3-yl)- 1,2,4-oxadiazol-5-yl)chroman-4- one |
| 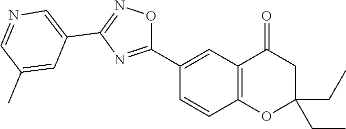 [SMILES: CCC1(CC)CC(=O)c2cc(-c3nc(-c4cncc(C)c4)no3)ccc2O1] | 563 | 2,2-diethyl-6-(3-(5- methylpyridin-3-yl)-1,2,4- oxadiazol-5-yl)chroman-4-one |
| 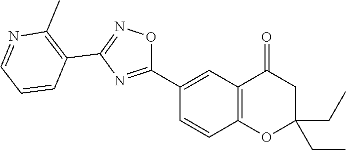 [SMILES: CCC1(CC)CC(=O)c2cc(-c3nc(-c4cccnc4C)no3)ccc2O1] | 564 | 2,2-diethyl-6-(3-(2- methylpyridin-3-yl)-1,2,4- oxadiazol-5-yl)chroman-4-one |
| 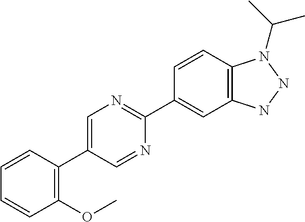 [SMILES: COc1ccccc1-c1cnc(-c2ccc3c(c2)NNN3C(C)C)nc1] | 565 | 1-isopropyl-5-(5-(2- methoxyphenyl)pyrimidin-2-yl)- 1H-benzo[d][1,2,3]triazole |
| 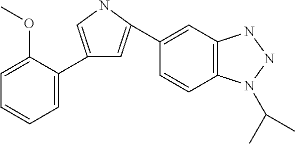 [SMILES: COc1ccccc1-c1c[nH]c(-c2ccc3c(c2)NNN3C(C)C)c1] | 566 | 5-[4-(2-methoxyphenyl)-1H- pyrrol-2-yl]-1-(propan-2-yl)-1H- 1,2,3-benzotriazole |
| 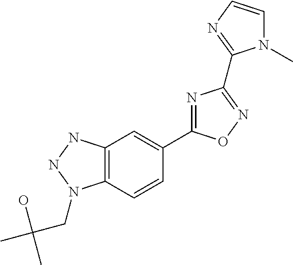 [SMILES: Cn1ccnc1-c1noc(-c2ccc3c(c2)NNN3CC(C)(C)O)n1] | 567 | 2-methyl-1-{5-[3-(1-methyl-1H- imidazol-2-yl)-1,2,4-oxadiazol-5- yl]-1H-1,2,3-benzotriazol-1- yl}propan-2-ol |
| 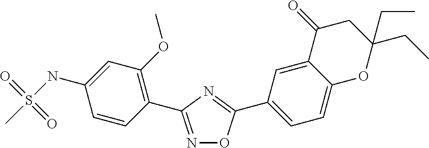 [SMILES: CCC1(CC)CC(=O)c2cc(-c3nc(-c4ccc(NS(C)(=O)=O)cc4OC)no3)ccc2O1] | 568 | N-{4-[5-(2,2-diethyl-4-oxo-3,4- dihydro-2H-1-benzopyran-6-yl)- 1,2,4-oxadiazol-3-yl]-3- methoxyphenyl} methanesulfonamide |
| 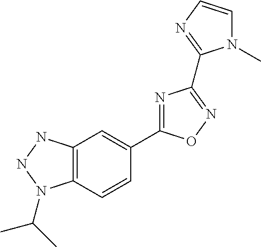 [SMILES: CC(C)N1NNc2cc(-c3nc(-c4nccn4C)no3)ccc21] | 569 | 5-[3-(1-methyl-1H-imidazol-2- yl)-1,2,4-oxadiazol-5-yl]-1- (propan-2-yl)-1H-1,2,3- benzotriazole |
| 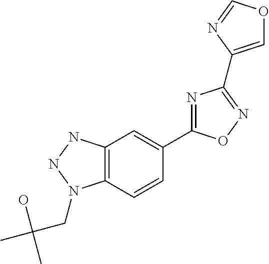 [SMILES: CC(C)(O)CN1NNc2cc(-c3nc(-c4cocn4)no3)ccc21] | 570 | 2-methyl-1-{5-[3-(1,3-oxazol-4- yl)-1,2,4-oxadiazol-5-yl]-1H- 1,2,3-benzotriazol-1-yl}propan-2- ol |
| 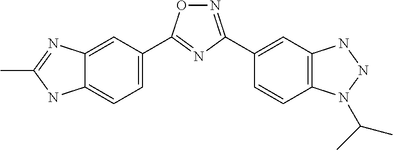 [SMILES: Cc1nc2cc(-c3nc(-c4ccc5c(c4)NNN5C(C)C)no3)ccc2[nH]1] | 571 | 5-[5-(2-methyl-1H-1,3- benzodiazol-5-yl)-1,2,4- oxadiazol-3-yl]-1-(propan-2-yl)- 1H-1,2,3-benzotriazole |
| 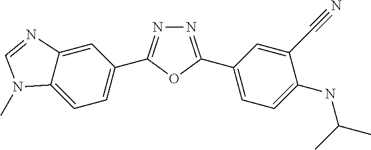 [SMILES: CC(C)Nc1ccc(-c2nnc(-c3ccc4c(c3)ncn4C)o2)cc1C#N] | 572 | 5-[5-(1-methyl-1H-1,3- benzodiazol-5-yl)-1,3,4- oxadiazol-2-yl]-2-[(propan-2- yl)amino]benzonitrile |
| 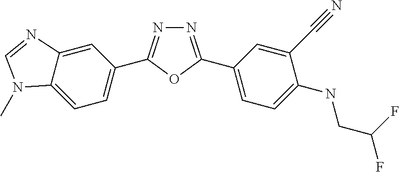 [SMILES: Cn1cnc2cc(-c3nnc(-c4ccc(NCC(F)F)c(C#N)c4)o3)ccc21] | 573 | 2-[(2,2-difluoroethyl)amino]-5-[5- (1-methyl-1H-1,3-benzodiazol-5- yl)-1,3,4-oxadiazol-2- yl]benzonitrile |
| 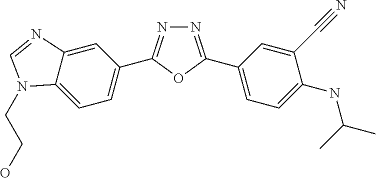 [SMILES: CC(C)Nc1ccc(-c2nnc(-c3ccc4c(c3)ncn4CCO)o2)cc1C#N] | 574 | 5-{5-[1-(2-hydroxyethyl)-1H-1,3- benzodiazol-5-yl]-1,3,4- oxadiazol-2-yl}-2-[(propan-2- yl)amino]benzonitrile |
| 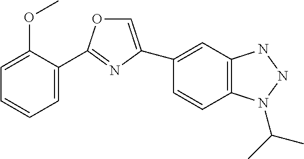 [SMILES: COc1ccccc1-c1nc(-c2ccc3c(c2)NNN3C(C)C)co1] | 575 | 5-[2-(2-methoxyphenyl)-1,3- oxazol-4-yl]-1-(propan-2-yl)-1H- 1,2,3-benzotriazole |
| 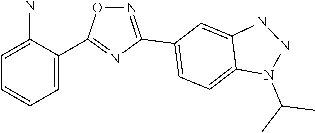 [SMILES: CC(C)N1NNc2cc(-c3noc(-c4ccccc4N)n3)ccc21] | 576 | 2-{3-[1-(propan-2-yl)-1H-1,2,3- benzotriazol-5-yl]-1,2,4- oxadiazol-5-yl}aniline |
| 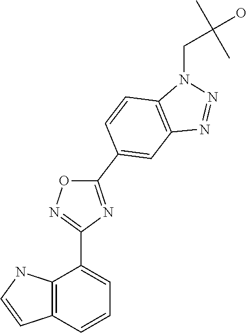 [SMILES: CC(C)(O)Cn1nnc2cc(-c3nc(-c4cccc5cc[nH]c45)no3)ccc21] | 577 | 1-{5-[3-(1H-indol-7-yl)-1,2,4- oxadiazol-5-yl]-1H-1,2,3- benzotriazol-1-yl}-2- methylpropan-2-ol |
| 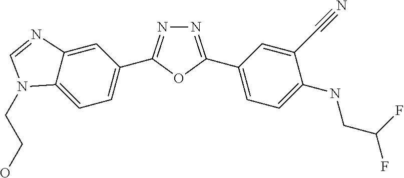 [SMILES: N#Cc1cc(-c2nnc(-c3ccc4c(c3)ncn4CCO)o2)ccc1NCC(F)F] | 578 | 2-[(2,2-difluoroethyl)amino]-5- {5-[1-(2-hydroxyethyl)-1H-1,3- benzodiazol-5-yl]-1,3,4- oxadiazol-2-yl}benzonitrile |
| 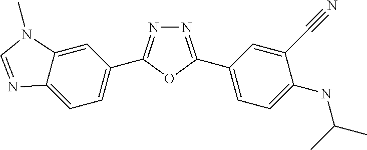 [SMILES: CC(C)Nc1ccc(-c2nnc(-c3ccc4ncn(C)c4c3)o2)cc1C#N] | 579 | 5-[5-(1-methyl-1H-1,3- benzodiazol-6-yl)-1,3,4- oxadiazol-2-yl]-2-[(propan-2- yl)amino]benzonitrile |
| 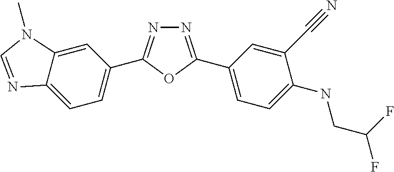 [SMILES: Cn1cnc2ccc(-c3nnc(-c4ccc(NCC(F)F)c(C#N)c4)o3)cc21] | 580 | 2-[(2,2-difluoroethyl)amino]-5-[5- (1-methyl-1H-1,3-benzodiazol-6- yl)-1,3,4-oxadiazol-2- yl]benzonitrile |
| 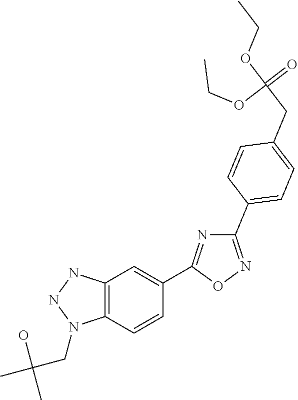 | 581 | [(4-{5-[1-(2-hydroxy-2- methylpropyl)-1H-1,2,3- benzotriazol-5-yl]-1,2,4- oxadiazol-3- yl}phenyl)methyl]phosphonate |
| 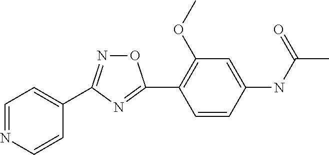 [SMILES: COc1cc(NC(C)=O)ccc1-c1nc(-c2ccncc2)no1] | 582 | N-{3-methoxy-4-[3-(pyridin-4- yl)-1,2,4-oxadiazol-5- yl]phenyl}acetamide |
| 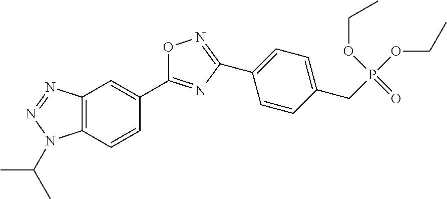 [SMILES: CCOP(=O)(Cc1ccc(-c2noc(-c3ccc4c(c3)nnn4C(C)C)n2)cc1)OCC] | 583 | diethyl [(4-{5-[1-(propan-2-yl)- 1H-1,2,3-benzotriazol-5-yl]- 1,2,4-oxadiazol-3- yl}phenyl)methyl]phosphonate |
| 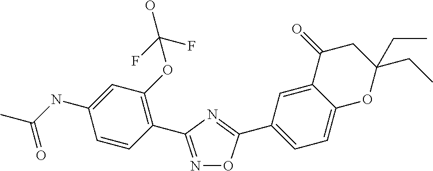 [SMILES: CCC1(CC)CC(=O)c2cc(-c3nc(-c4ccc(NC(C)=O)cc4OC(O)(F)F)no3)ccc2O1] | 584 | N-{4-[5-(2,2-diethyl-4-oxo-3,4- dihydro-2H-1-benzopyran-6-yl)- 1,2,4-oxadiazol-3-yl]-3- (trifluoromethoxy)phenyl} acetamide |
| 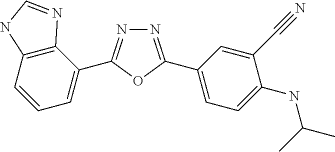 [SMILES: CC(C)Nc1ccc(-c2nnc(-c3cccc4[nH]cnc34)o2)cc1C#N] | 585 | 5-[5-(1H-1,3-benzodiazol-4-yl)- 1,3,4-oxadiazol-2-yl]-2-[(propan- 2-yl)amino]benzonitrile |
| 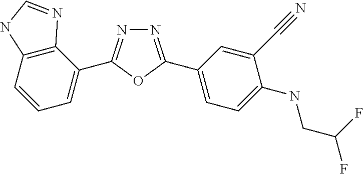 [SMILES: N#Cc1cc(-c2nnc(-c3cccc4[nH]cnc34)o2)ccc1NCC(F)F] | 586 | 5-[5-(1H-1,3-benzodiazol-4-yl)- 1,3,4-oxadiazol-2-yl]-2-[(2,2- difluoroethyl)amino]benzonitrile |
| 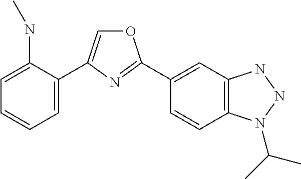 [SMILES: CNc1ccccc1-c1coc(-c2ccc3c(c2)NNN3C(C)C)n1] | 587 | N-methyl-2-{2-[1-(propan-2-yl)- 1H-1,2,3-benzotriazol-5-yl]-1,3- oxazol-4-yl}aniline |
| 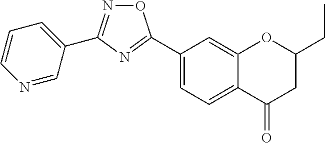 [SMILES: CCC1CC(=O)c2ccc(-c3nc(-c4cccnc4)no3)cc2O1] | 588 | 2-ethyl-7-[3-(pyridin-3-yl)-1,2,4- oxadiazol-5-yl]-3,4-dihydro-2H- 1-benzopyran-4-one |
| 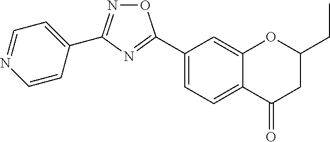 [SMILES: CCC1CC(=O)c2ccc(-c3nc(-c4ccncc4)no3)cc2O1] | 589 | 2-ethyl-7-[3-(pyridin-4-yl)-1,2,4- oxadiazol-5-yl]-3,4-dihydro-2H- 1-benzopyran-4-one |
| 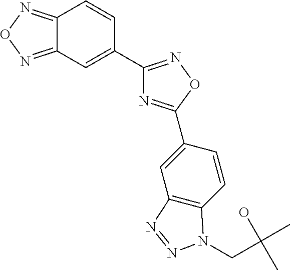 [SMILES: CC(C)(O)Cn1nnc2cc(-c3nc(-c4ccc5nonc5c4)no3)ccc21] | 590 | 1-{5-[3-(2,1,3-benzoxadiazol-5- yl)-1,2,4-oxadiazol-5-yl]-1H- 1,2,3-benzotriazol-1-yl}-2- methylpropan-2-ol |
| 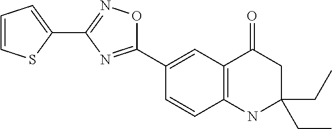 [SMILES: CCC1(CC)CC(=O)c2cc(-c3nc(-c4cccs4)no3)ccc2N1] | 591 | 2,2-diethyl-6-(3-(thiophen-2-yl)- 1,2,4-oxadiazol-5-yl)-2,3- dihydroquinolin-4(1H)-one |
| 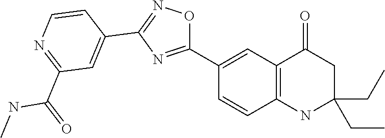 [SMILES: CCC1(CC)CC(=O)c2cc(-c3nc(-c4ccnc(C(=O)NC)c4)no3)ccc2N1] | 592 | 4-(5-(2,2-diethyl-4-oxochroman- 6-yl)-1,2,4-oxadiazol-3-yl)-N- methylpicolinamide |
| 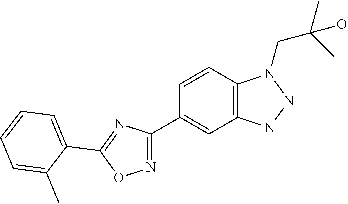 [SMILES: Cc1ccccc1-c1nc(-c2ccc3c(c2)NNN3CC(C)(C)O)no1] | 593 | 2-methyl-1-(5-(5-(o-tolyl)-1,2,4- oxadiazol-3-yl)-1H- benzo[d][1,2,3]triazol-1- yl)propan-2-ol |
| 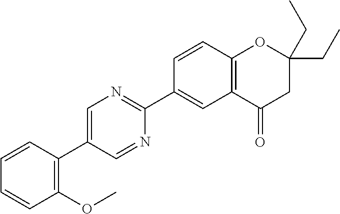 [SMILES: CCC1(CC)CC(=O)c2cc(-c3ncc(-c4ccccc4OC)cn3)ccc2O1] | 594 | 2,2-diethyl-6-(5-(2- methoxyphenyl)pyrimidin-2- yl)chroman-4-one |
| 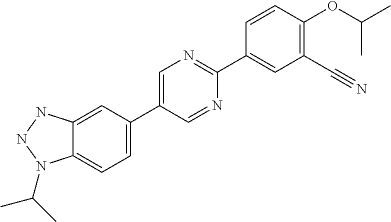 [SMILES: CC(C)Oc1ccc(-c2ncc(-c3ccc4c(c3)NNN4C(C)C)cn2)cc1C#N] | 595 | 2-isopropoxy-5-(5-(1-isopropyl- 1H-benzo[d][1,2,3]triazol-5- yl)pyrimidin-2-yl)benzonitrile |
| 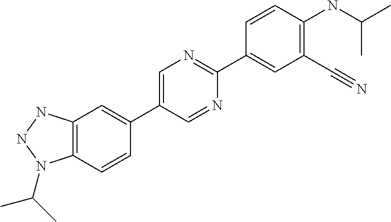 [SMILES: CC(C)Nc1ccc(-c2ncc(-c3ccc4c(c3)NNN4C(C)C)cn2)cc1C#N] | 596 | 5-(5-(1-isopropyl-1H- benzo[d][1,2,3]triazol-5- yl)pyrimidin-2-yl)-2- (isopropylamino)benzonitrile |
| 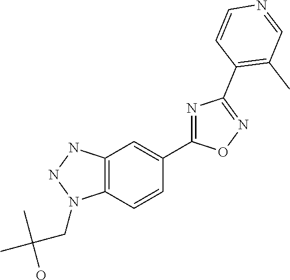 [SMILES: Cc1cnccc1-c1noc(-c2ccc3c(c2)NNN3CC(C)(C)O)n1] | 597 | 2-methyl-1-{5-[3-(3- methylpyridin-4-yl)-1,2,4- oxadiazol-5-yl]-1H-1,2,3- benzotriazol-1-yl}propan-2-ol |
| 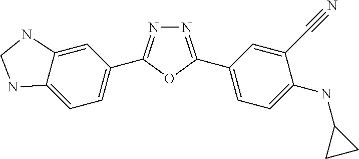 [SMILES: N#Cc1cc(-c2nnc(-c3ccc4c(c3)NCN4)o2)ccc1NC1CC1] | 598 | 5-[5-(1H-1,3-benzodiazol-5-yl)- 1,3,4-oxadiazol-2-yl]-2- (cyclopropylamino)benzonitrile |
| 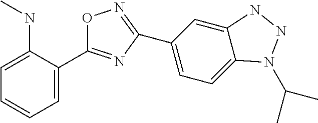 [SMILES: CNc1ccccc1-c1nc(-c2ccc3c(c2)NNN3C(C)C)no1] | 599 | N-methyl-2-{3-[1-(propan-2-yl)- 1H-1,2,3-benzotriazol-5-yl]- 1,2,4-oxadiazol-5-yl}aniline |
| 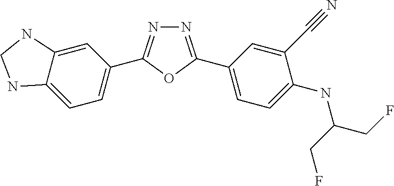 [SMILES: N#Cc1cc(-c2nnc(-c3ccc4c(c3)NCN4)o2)ccc1NC(CF)CF] | 600 | 5-[5-(1H-1,3-benzodiazol-5-yl)- 1,3,4-oxadiazol-2-yl]-2-[(1,3- difluoropropan-2- yl)amino]benzonitrile |
| 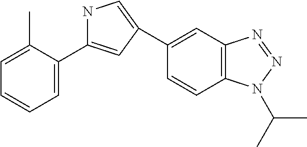 [SMILES: Cc1ccccc1-c1cc(-c2ccc3c(c2)nnn3C(C)C)c[nH]1] | 601 | 5-[5-(2-methylphenyl)-1H-pyrrol- 3-yl]-1-(propan-2-yl)-1H-1,2,3- benzotriazole |
| 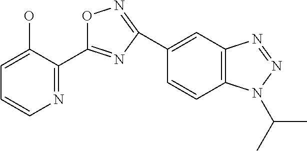 [SMILES: CC(C)n1nnc2cc(-c3noc(-c4ncccc4O)n3)ccc21] | 602 | 2-{3-[1-(propan-2-yl)-1H-1,2,3- benzotriazol-5-yl]-1,2,4- oxadiazol-5-yl}pyridin-3-ol |
| 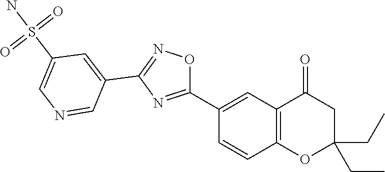 [SMILES: CCC1(CC)CC(=O)c2cc(-c3nc(-c4cncc(S(N)(=O)=O)c4)no3)ccc2O1] | 603 | 5-(5-(2,2-diethyl-4-oxochroman- 6-yl)-1,2,4-oxadiazol-3- yl)pyridine-3-sulfonamide |
| 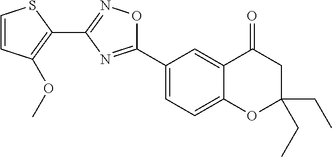 [SMILES: CCC1(CC)CC(=O)c2cc(-c3nc(-c4sccc4OC)no3)ccc2O1] | 604 | 2,2-diethyl-6-(3-(3- methoxythiophen-2-yl)-1,2,4- oxadiazol-5-yl)chroman-4-one |
| 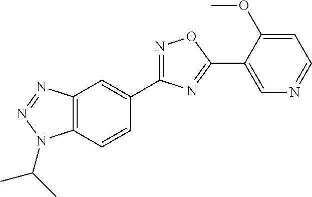 [SMILES: COc1ccncc1-c1nc(-c2ccc3c(c2)nnn3C(C)C)no1] | 605 | 3-(1-isopropyl-1H- benzo[d][1,2,3]triazol-5-yl)-5-(4- methoxypyridin-3-yl)-1,2,4- oxadiazole |
| 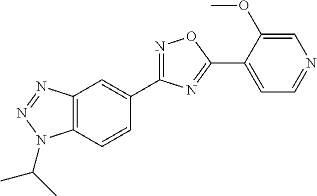 [SMILES: COc1cnccc1-c1nc(-c2ccc3c(c2)nnn3C(C)C)no1] | 606 | 3-(1-isopropyl-1H- benzo[d][1,2,3]triazol-5-yl)-5-(3- methoxypyridin-4-yl)-1,2,4- oxadiazole |
| 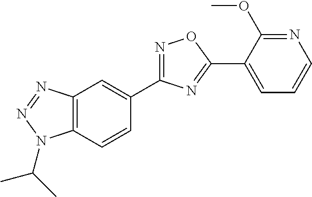 [SMILES: COc1ncccc1-c1nc(-c2ccc3c(c2)nnn3C(C)C)no1] | 607 | 3-(1-isopropyl-1H- benzo[d][1,2,3]triazol-5-yl)-5-(2- methoxypyridin-3-yl)-1,2,4- oxadiazole |
| 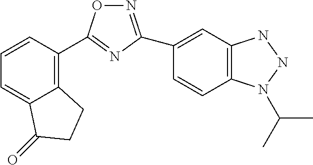 [SMILES: CC(C)N1NNc2cc(-c3noc(-c4cccc5c4CCC5=O)n3)ccc21] | 608 | 4-{3-[1-(propan-2-yl)-1H-1,2,3- benzotriazol-5-yl]-1,2,4- oxadiazol-5-yl}-2,3-dihydro-1H- inden-1-one |
| 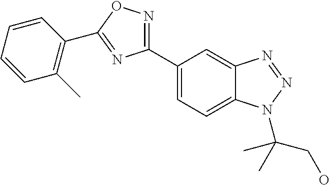 [SMILES: Cc1ccccc1-c1nc(-c2ccc3c(c2)nnn3C(C)(C)CO)no1] | 609 | 2-methyl-2-(5-(5-(o-tolyl)-1,2,4- oxadiazol-3-yl)-1H- benzo[d][1,2,3]triazol-1- yl)propan-1-ol |
| 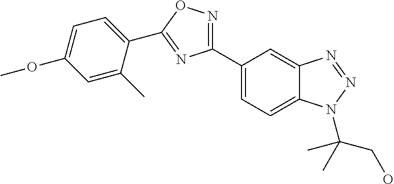 [SMILES: COc1ccc(-c2nc(-c3ccc4c(c3)nnn4C(C)(C)CO)no2)c(C)c1] | 610 | 2-(5-(5-(4-methoxy-2- methylphenyl)-1,2,4-oxadiazol-3- yl)-1H-benzo[d][1,2,3]triazol-1- yl)-2-methylpropan-1-ol |
| 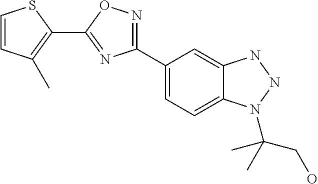 [SMILES: Cc1ccsc1-c1nc(-c2ccc3c(c2)NNN3C(C)(C)CO)no1] | 611 | 2-methyl-2-(5-(5-(3- methylthiophen-2-yl)-1,2,4- oxadiazol-3-yl)-1H- benzo[d][1,2,3]triazol-1- yl)propan-1-ol |
| 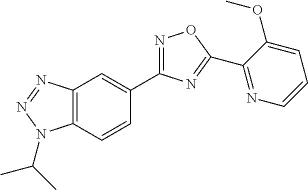 [SMILES: COc1cccnc1-c1nc(-c2ccc3c(c2)nnn3C(C)C)no1] | 612 | 3-(1-isopropyl-1H- benzo[d][1,2,3]triazol-5-yl)-5-(3- methoxypyridin-2-yl)-1,2,4- oxadiazole |
| 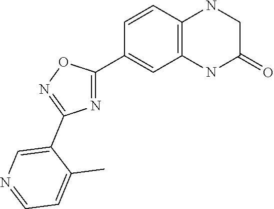 [SMILES: Cc1ccncc1-c1noc(-c2ccc3c(c2)NC(=O)CN3)n1] | 613 | 7-[3-(4-methylpyridin-3-yl)-1,2,4- oxadiazol-5-yl]-1,2,3,4- tetrahydroquinoxalin-2-one |
| 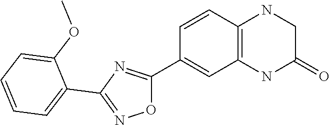 [SMILES: COc1ccccc1-c1noc(-c2ccc3c(c2)NC(=O)CN3)n1] | 614 | 7-[3-(2-methoxyphenyl)-1,2,4- oxadiazol-5-yl]-1,2,3,4- tetrahydroquinoxalin-2-one |
| 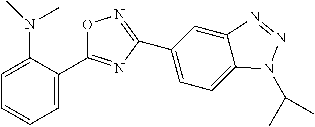 [SMILES: CC(C)n1nnc2cc(-c3noc(-c4ccccc4N(C)C)n3)ccc21] | 615 | N,N-dimethyl-2-{3-[1-(propan-2- yl)-1H-1,2,3-benzotriazol-5-yl]- 1,2,4-oxadiazol-5-yl}aniline |
| 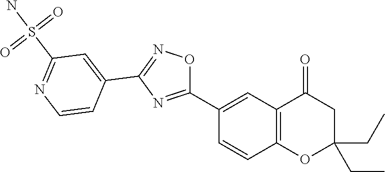 [SMILES: CCC1(CC)CC(=O)c2cc(-c3nc(-c4ccnc(S(N)(=O)=O)c4)no3)ccc2O1] | 616 | 4-(5-(2,2-diethyl-4-oxochroman- 6-yl)-1,2,4-oxadiazol-3- yl)pyridine-2-sulfonamide |
| 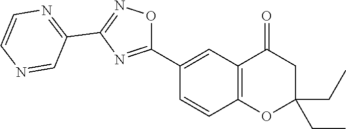 [SMILES: CCC1(CC)CC(=O)c2cc(-c3nc(-c4cnccn4)no3)ccc2O1] | 617 | 2,2-diethyl-6-(3-(pyrazin-2-yl)- 1,2,4-oxadiazol-5-yl)chroman-4- one |
| 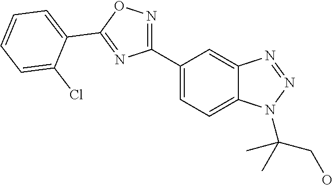 [SMILES: CC(C)(CO)n1nnc2cc(-c3noc(-c4ccccc4Cl)n3)ccc21] | 618 | 2-(5-(5-(2-chlorophenyl)-1,2,4- oxadiazol-3-yl)-1H- benzo[d][1,2,3]triazol-1-yl)-2- methylpropan-1-ol |
| 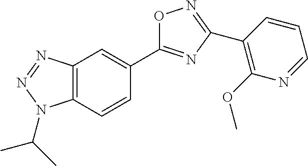 [SMILES: COc1ncccc1-c1noc(-c2ccc3c(c2)nnn3C(C)C)n1] | 619 | 5-(1-isopropyl-1H- benzo[d][1,2,3]triazol-5-yl)-3-(2- methoxypyridin-3-yl)-1,2,4- oxadiazole |
| 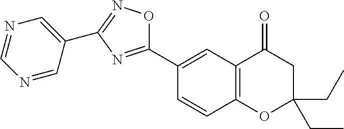 [SMILES: CCC1(CC)CC(=O)c2cc(-c3nc(-c4cncnc4)no3)ccc2O1] | 620 | 2,2-diethyl-6-(3-(pyrimidin-5-yl)- 1,2,4-oxadiazol-5-yl)chroman-4- one |
| 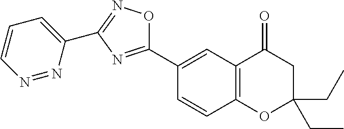 [SMILES: CCC1(CC)CC(=O)c2cc(-c3nc(-c4cccnn4)no3)ccc2O1] | 621 | 2,2-diethyl-6-(3-(pyridazin-3-yl)- 1,2,4-oxadiazol-5-yl)chroman-4- one |
| 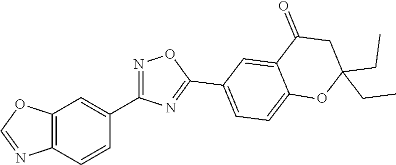 [SMILES: CCC1(CC)CC(=O)c2cc(-c3nc(-c4ccc5ncoc5c4)no3)ccc2O1] | 622 | 6-(3-(benzo[d]oxazol-6-yl)-1,2,4- oxadiazol-5-yl)-2,2- diethylchroman-4-one |
| 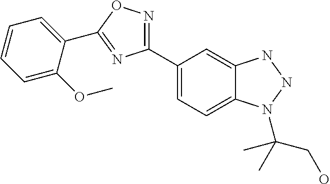 [SMILES: COc1ccccc1-c1nc(-c2ccc3c(c2)NNN3C(C)(C)CO)no1] | 623 | 2-(5-(5-(2-methoxyphenyl)-1,2,4- oxadiazol-3-yl)-1H- benzo[d][1,2,3]triazol-1-yl)-2- methylpropan-1-ol |
| 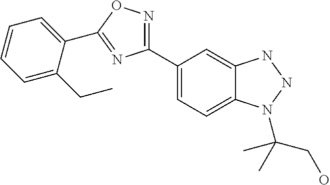 [SMILES: CCc1ccccc1-c1nc(-c2ccc3c(c2)NNN3C(C)(C)CO)no1] | 624 | 2-(5-(5-(2-ethylphenyl)-1,2,4- oxadiazol-3-yl)-1H- benzo[d][1,2,3]triazol-1-yl)-2- methylpropan-1-ol |
| 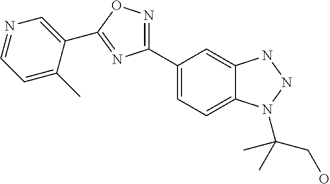 [SMILES: Cc1ccncc1-c1nc(-c2ccc3c(c2)NNN3C(C)(C)CO)no1] | 625 | 2-methyl-2-(5-(5-(4- methylpyridin-3-yl)-1,2,4- oxadiazol-3-yl)-1H- benzo[d][1,2,3]triazol-1- yl)propan-1-ol |
| 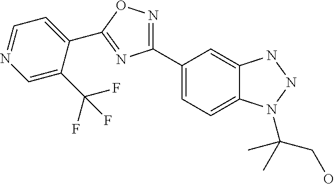 [SMILES: CC(C)(CO)N1NNc2cc(-c3noc(-c4ccncc4C(F)(F)F)n3)ccc21] | 626 | 2-methyl-2-(5-(5-(3- (trifluoromethyl)pyridin-4-yl)- 1,2,4-oxadiazol-3-yl)-1H- benzo[d][1,2,3]triazol-1- yl)propan-1-ol |
| 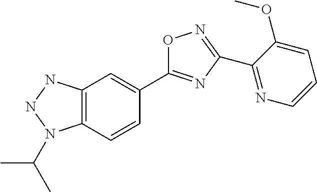 [SMILES: COc1cccnc1-c1noc(-c2ccc3c(c2)NNN3C(C)C)n1] | 627 | 5-(1-isopropyl-1H- benzo[d][1,2,3]triazol-5-yl)-3-(3- methoxypyridin-2-yl)-1,2,4- oxadiazole |
| 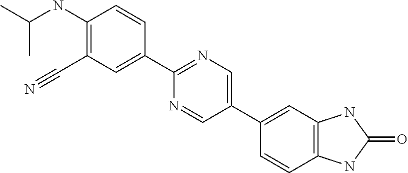 [SMILES: CC(C)Nc1ccc(-c2ncc(-c3ccc4[nH]c(=O)[nH]c4c3)cn2)cc1C#N] | 628 | 2-(isopropylamino)-5-(5-(2-oxo- 2,3-dihydro-1H- benzo[d]imidazol-5-yl)pyrimidin- 2-yl)benzonitrile |
| 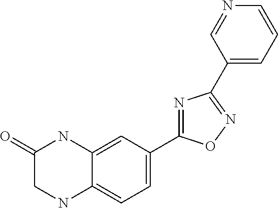 [SMILES: O=C1CNc2ccc(-c3nc(-c4cccnc4)no3)cc2N1] | 629 | 7-[3-(pyridin-3-yl)-1,2,4- oxadiazol-5-yl]-1,2,3,4- tetrahydroquinoxalin-2-one |
| 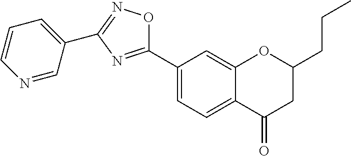 [SMILES: CCCC1CC(=O)c2ccc(-c3nc(-c4cccnc4)no3)cc2O1] | 630 | 2-propyl-7-[3-(pyridin-3-yl)- 1,2,4-oxadiazol-5-yl]-3,4- dihydro-2H-1-benzopyran-4-one |
| 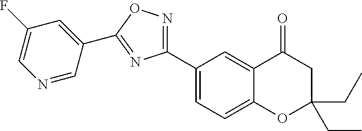 [SMILES: CCC1(CC)CC(=O)c2cc(-c3noc(-c4cncc(F)c4)n3)ccc2O1] | 631 | 2,2-diethyl-6-[5-(5-fluoropyridin- 3-yl)-1,2,4-oxadiazol-3-yl]-3,4- dihydro-2H-1-benzopyran-4-one |
| 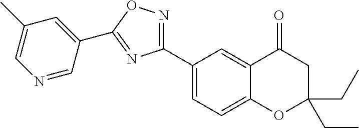 [SMILES: CCC1(CC)CC(=O)c2cc(-c3noc(-c4cncc(C)c4)n3)ccc2O1] | 632 | 2,2-diethyl-6-[5-(5- methylpyridin-3-yl)-1,2,4- oxadiazol-3-yl]-3,4-dihydro-2H- 1-benzopyran-4-one |
| 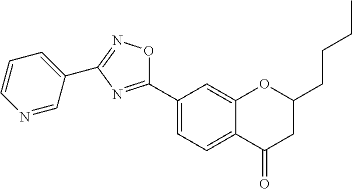 [SMILES: CCCCC1CC(=O)c2ccc(-c3nc(-c4cccnc4)no3)cc2O1] | 633 | 2-butyl-7-[3-(pyridin-3-yl)-1,2,4- oxadiazol-5-yl]-3,4-dihydro-2H- 1-benzopyran-4-one |
| 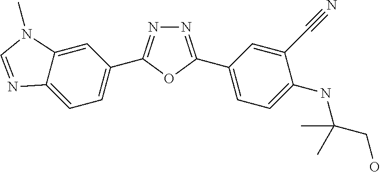 [SMILES: Cn1cnc2ccc(-c3nnc(-c4ccc(NC(C)(C)CO)c(C#N)c4)o3)cc21] | 634 | 2-[(1-hydroxy-2-methylpropan-2- yl)amino]-5-[5-(1-methyl-1H- 1,3-benzodiazol-6-yl)-1,3,4- oxadiazol-2-yl]benzonitrile |
| 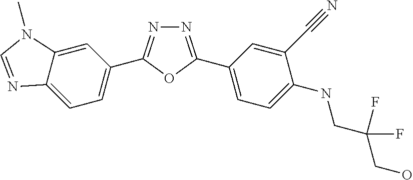 [SMILES: Cn1cnc2ccc(-c3nnc(-c4ccc(NCC(F)(F)CO)c(C#N)c4)o3)cc21] | 635 | 2-[(2,2-difluoro-3- hydroxypropyl)amino]-5-[5-(1- methyl-1H-1,3-benzodiazol-6-yl)- 1,3,4-oxadiazol-2-yl]benzonitrile |
| 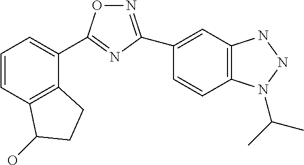 [SMILES: CC(C)N1NNc2cc(-c3noc(-c4cccc5c4CCC5O)n3)ccc21] | 636 | 4-{3-[1-(propan-2-yl)-1H-1,2,3- benzotriazol-5-yl]-1,2,4- oxadiazol-5-yl}-2,3-dihydro-1H- inden-1-ol |
| 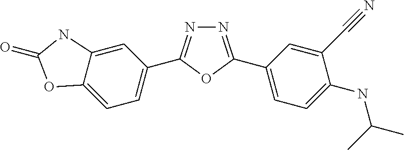 [SMILES: CC(C)Nc1ccc(-c2nnc(-c3ccc4oc(=O)[nH]c4c3)o2)cc1C#N] | 637 | 5-[5-(2-oxo-2,3-dihydro-1,3- benzoxazol-5-yl)-1,3,4- oxadiazol-2-yl]-2-[(propan-2- yl)amino]benzonitrile |
| 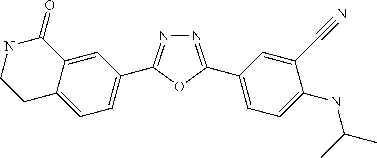 [SMILES: CC(C)Nc1ccc(-c2nnc(-c3ccc4c(c3)C(=O)NCC4)o2)cc1C#N] | 638 | 5-[5-(1-oxo-1,2,3,4- tetrahydroisoquinolin-7-yl)-1,3,4- oxadiazol-2-yl]-2-[(propan-2- yl)amino]benzonitrile |
| 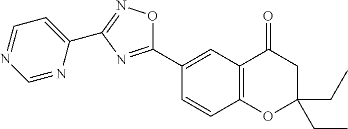 [SMILES: CCC1(CC)CC(=O)c2cc(-c3nc(-c4ccncn4)no3)ccc2O1] | 639 | 2,2-diethyl-6-(3-(pyrimidin-4-yl)- 1,2,4-oxadiazol-5-yl)chroman-4- one |
| 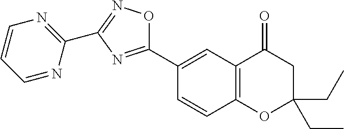 [SMILES: CCC1(CC)CC(=O)c2cc(-c3nc(-c4ncccn4)no3)ccc2O1] | 640 | 2,2-diethyl-6-(3-(pyrimidin-2-yl)- 1,2,4-oxadiazol-5-yl)chroman-4- one |
| 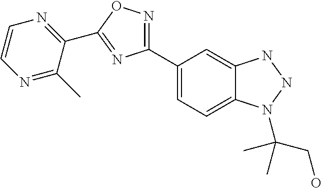 [SMILES: Cc1nccnc1-c1nc(-c2ccc3c(c2)NNN3C(C)(C)CO)no1] | 641 | 2-methyl-2-(5-(5-(3- methylpyrazin-2-yl)-1,2,4- oxadiazol-3-yl)-1H- benzo[d][1,2,3]triazol-1- yl)propan-1-ol |
| 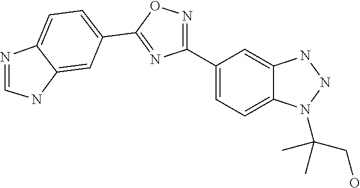 [SMILES: CC(C)(CO)N1NNc2cc(-c3noc(-c4ccc5nc[nH]c5c4)n3)ccc21] | 642 | 2-(5-(5-(1H-benzo[d]imidazol-6- yl)-1,2,4-oxadiazol-3-yl)-1H- benzo[d][1,2,3]triazol-1-yl)-2- methylpropan-1-ol |
| 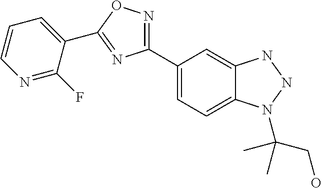 [SMILES: CC(C)(CO)N1NNc2cc(-c3noc(-c4cccnc4F)n3)ccc21] | 643 | 2-(5-(5-(2-fluoropyridin-3-yl)- 1,2,4-oxadiazol-3-yl)-1H- benzo[d][1,2,3]triazol-1-yl)-2- methylpropan-1-ol |
| 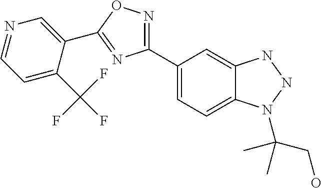 [SMILES: CC(C)(CO)N1NNc2cc(-c3noc(-c4cnccc4C(F)(F)F)n3)ccc21] | 644 | 2-methyl-2-(5-(5-(4- (trifluoromethyl)pyridin-3-yl)- 1,2,4-oxadiazol-3-yl)-1H- benzo[d][1,2,3]triazol-1- yl)propan-1-ol |
| 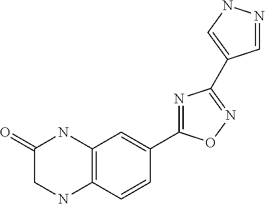 [SMILES: O=C1CNc2ccc(-c3nc(-c4cn[nH]c4)no3)cc2N1] | 645 | 7-[3-(1H-pyrazol-4-yl)-1,2,4- oxadiazol-5-yl]-1,2,3,4- tetrahydroquinoxalin-2-one |
| 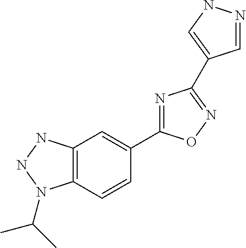 [SMILES: CC(C)N1NNc2cc(-c3nc(-c4cn[nH]c4)no3)ccc21] | 646 | 1-(propan-2-yl)-5-[3-(1H-pyrazol- 4-yl)-1,2,4-oxadiazol-5-yl]-1H- 1,2,3-benzotriazole |
| 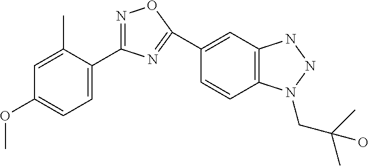 [SMILES: COc1ccc(-c2noc(-c3ccc4c(c3)NNN4CC(C)(C)O)n2)c(C)c1] | 647 | 1-{5-[3-(4-methoxy-2- methylphenyl)-1,2,4-oxadiazol-5- yl]-1H-1,2,3-benzotriazol-1-yl}- 2-methylpropan-2-ol |
| 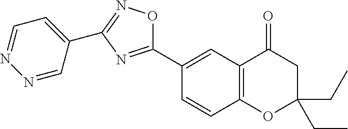 [SMILES: CCC1(CC)CC(=O)c2cc(-c3nc(-c4ccnnc4)no3)ccc2O1] | 648 | 2,2-diethyl-6-(3-(pyridazin-4-yl)- 1,2,4-oxadiazol-5-yl)chroman-4- one |
| 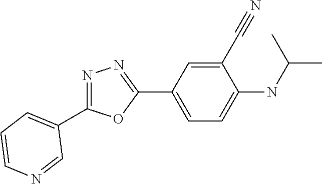 [SMILES: CC(C)Nc1ccc(-c2nnc(-c3cccnc3)o2)cc1C#N] | 649 | 2-(isopropylamino)-5-(5-(pyridin- 3-yl)-1,3,4-oxadiazol-2- yl)benzonitrile |
| 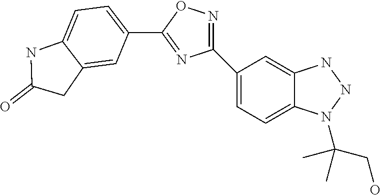 [SMILES: CC(C)(CO)N1NNc2cc(-c3noc(-c4ccc5c(c4)CC(=O)N5)n3)ccc21] | 650 | 5-(3-(1-(1-hydroxy-2- methylpropan-2-yl)-1H- benzo[d][1,2,3]triazol-5-yl)-1,2,4- oxadiazol-5-yl)indolin-2-one |
| 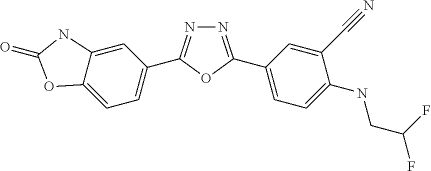 [SMILES: N#Cc1cc(-c2nnc(-c3ccc4oc(=O)[nH]c4c3)o2)ccc1NCC(F)F] | 651 | 2-[(2,2-difluoroethyl)amino]-5-[5- (2-oxo-2,3-dihydro-1,3- benzoxazol-5-yl)-1,3,4-oxadiazol- 2-yl]benzonitrile |
| 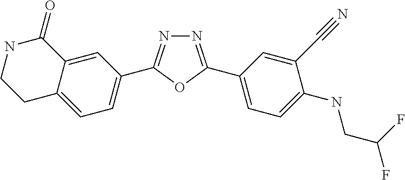 [SMILES: N#Cc1cc(-c2nnc(-c3ccc4c(c3)C(=O)NCC4)o2)ccc1NCC(F)F] | 652 | 2-[(2,2-difluoroethyl)amino]-5-[5- (1-oxo-1,2,3,4- tetrahydroisoquinolin-7-yl)-1,3,4- oxadiazol-2-yl]benzonitrile |
| 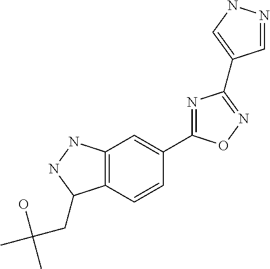 [SMILES: CC(C)(O)CC1NNc2cc(-c3nc(-c4cn[nH]c4)no3)ccc21] | 653 | 2-methyl-1-{6-[3-(1H-pyrazol-4- yl)-1,2,4-oxadiazol-5-yl]-3H- indazol-3-yl}propan-2-ol |
| 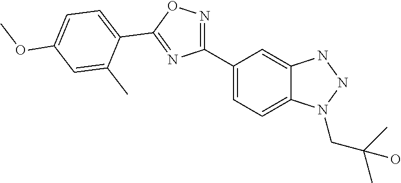 [SMILES: COc1ccc(-c2nc(-c3ccc4c(c3)NNN4CC(C)(C)O)no2)c(C)c1] | 654 | 1-(5-(5-(4-methoxy-2- methylphenyl)-1,2,4-oxadiazol-3- yl)-1H-benzo[d][1,2,3]triazol-1- yl)-2-methylpropan-2-ol |
| 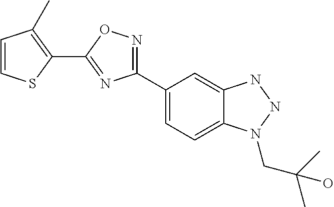 [SMILES: Cc1ccsc1-c1nc(-c2ccc3c(c2)NNN3CC(C)(C)O)no1] | 655 | 2-methyl-1-(5-(5-(3- methylthiophen-2-yl)-1,2,4- oxadiazol-3-yl)-1H- benzo[d][1,2,3]triazol-1- yl)propan-2-ol |
| 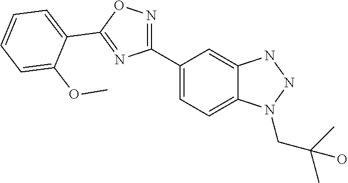 [SMILES: COc1ccccc1-c1nc(-c2ccc3c(c2)NNN3CC(C)(C)O)no1] | 656 | 1-(5-(5-(2-methoxyphenyl)-1,2,4- oxadiazol-3-yl)-1H- benzo[d][1,2,3]triazol-1-yl)-2- methylpropan-2-ol |
| 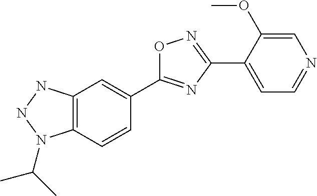 [SMILES: COc1cnccc1-c1noc(-c2ccc3c(c2)NNN3C(C)C)n1] | 657 | 5-(1-isopropyl-1H- benzo[d][1,2,3]triazol-5-yl)-3-(3- methoxypyridin-4-yl)-1,2,4- oxadiazole |
| 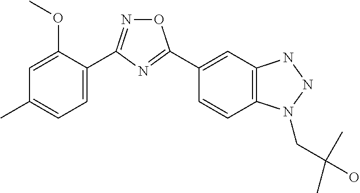 [SMILES: COc1cc(C)ccc1-c1noc(-c2ccc3c(c2)NNN3CC(C)(C)O)n1] | 662 | 1-{5-[3-(2-methoxy-4- methylphenyl)-1,2,4-oxadiazol-5- yl]-1H-1,2,3-benzotriazol-1-yl}- 2-methylpropan-2-ol |
| 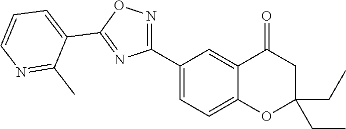 [SMILES: CCC1(CC)CC(=O)c2cc(-c3noc(-c4cccnc4C)n3)ccc2O1] | 663 | 2,2-diethyl-6-[5-(2- methylpyridin-3-yl)-1,2,4- oxadiazol-3-yl]-3,4-dihydro-2H- 1-benzopyran-4-one |
| 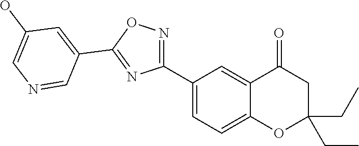 [SMILES: CCC1(CC)CC(=O)c2cc(-c3noc(-c4cncc(O)c4)n3)ccc2O1] | 664 | 2,2-diethyl-6-[5-(5- hydroxypyridin-3-yl)-1,2,4- oxadiazol-3-yl]-3,4-dihydro-2H- 1-benzopyran-4-one |
| 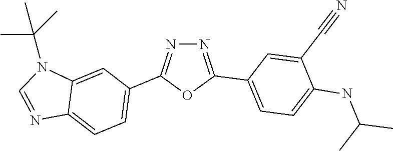 [SMILES: CC(C)Nc1ccc(-c2nnc(-c3ccc4ncn(C(C)(C)C)c4c3)o2)cc1C#N] | 665 | 5-[5-(1-tert-butyl-1H-1,3- benzodiazol-6-yl)-1,3,4- oxadiazol-2-yl]-2-[(propan-2- yl)amino]benzonitrile |
| 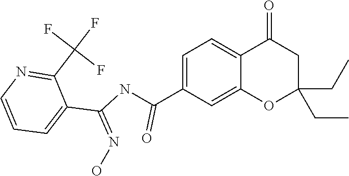 [SMILES: CCC1(CC)CC(=O)c2ccc(C(=O)N/C(=N/O)c3cccnc3C(F)(F)F)cc2O1] | 666 | 2,2-diethyl-N-[(1E)- (hydroxyimino)[2- (trifluoromethyl)pyridin-3- yl]methyl]-4-oxo-3,4-dihydro- 2H-1-benzopyran-7-carboxamide |
| 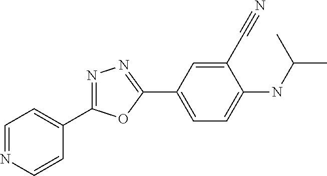 [SMILES: CC(C)Nc1ccc(-c2nnc(-c3ccncc3)o2)cc1C#N] | 667 | 2-(isopropylamino)-5-(5-(pyridin- 4-yl)-1,3,4-oxadiazol-2- yl)benzonitrile |
| 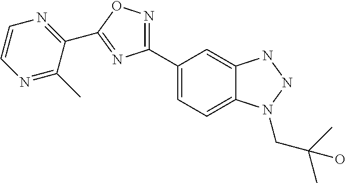 [SMILES: Cc1nccnc1-c1nc(-c2ccc3c(c2)NNN3CC(C)(C)O)no1] | 668 | 2-methyl-1-(5-(5-(3- methylpyrazin-2-yl)-1,2,4- oxadiazol-3-yl)-1H- benzo[d][1,2,3]triazol-1- yl)propan-2-ol |
| 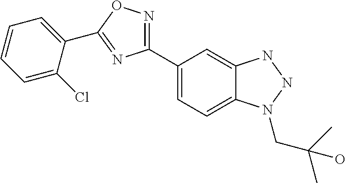 [SMILES: CC(C)(O)CN1NNc2cc(-c3noc(-c4ccccc4Cl)n3)ccc21] | 669 | 1-(5-(5-(2-chlorophenyl)-1,2,4- oxadiazol-3-yl)-1H- benzo[d][1,2,3]triazol-1-yl)-2- methylpropan-2-ol |
| 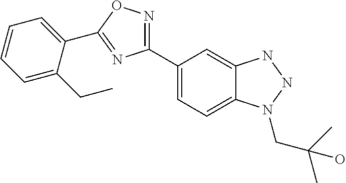 [SMILES: CCc1ccccc1-c1nc(-c2ccc3c(c2)NNN3CC(C)(C)O)no1] | 670 | 1-(5-(5-(2-ethylphenyl)-1,2,4- oxadiazol-3-yl)-1H- benzo[d][1,2,3]triazol-1-yl)-2- methylpropan-2-ol |
| 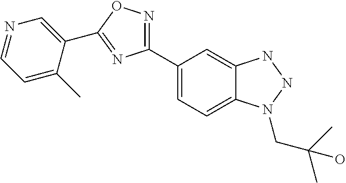 [SMILES: Cc1ccncc1-c1nc(-c2ccc3c(c2)NNN3CC(C)(C)O)no1] | 671 | 2-methyl-1-(5-(5-(4- methylpyridin-3-yl)-1,2,4- oxadiazol-3-yl)-1H- benzo[d][1,2,3]triazol-1- yl)propan-2-ol |
| 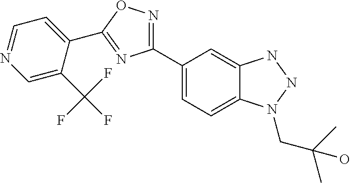 [SMILES: CC(C)(O)CN1NNc2cc(-c3noc(-c4ccncc4C(F)(F)F)n3)ccc21] | 672 | 2-methyl-1-(5-(5-(3- (trifluoromethyl)pyridin-4-yl)- 1,2,4-oxadiazol-3-yl)-1H- benzo[d][1,2,3]triazol-1- yl)propan-2-ol |
| 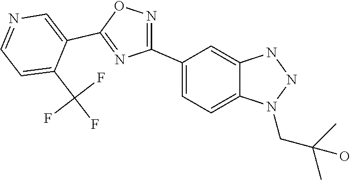 [SMILES: CC(C)(O)CN1NNc2cc(-c3noc(-c4cnccc4C(F)(F)F)n3)ccc21] | 673 | 2-methyl-1-(5-(5-(4- (trifluoromethyl)pyridin-3-yl)- 1,2,4-oxadiazol-3-yl)-1H- benzo[d][1,2,3]triazol-1- yl)propan-2-ol |
| 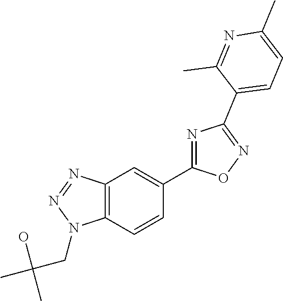 [SMILES: Cc1ccc(-c2noc(-c3ccc4c(c3)nnn4CC(C)(C)O)n2)c(C)n1] | 675 | 1-{5-[3-(2,6-dimethylpyridin-3- yl)-1,2,4-oxadiazol-5-yl]-1H- 1,2,3-benzotriazol-1-yl}-2- methylpropan-2-ol |
| 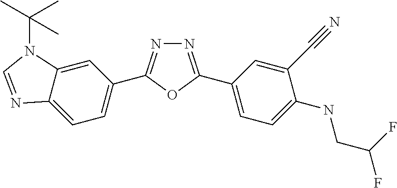 [SMILES: CC(C)(C)n1cnc2ccc(-c3nnc(-c4ccc(NCC(F)F)c(C#N)c4)o3)cc21] | 676 | 5-[5-(1-tert-butyl-1H-1,3- benzodiazol-6-yl)-1,3,4- oxadiazol-2-yl]-2-[(2,2- difluoroethyl)amino]benzonitrile |
| 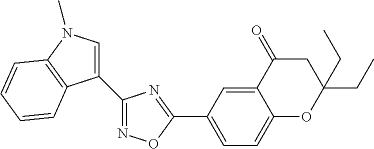 [SMILES: CCC1(CC)CC(=O)c2cc(-c3nc(-c4cn(C)c5ccccc45)no3)ccc2O1] | 677 | 2,2-diethyl-6-[3-(1-methyl-1H- indol-3-yl)-1,2,4-oxadiazol-5-yl]- 3,4-dihydro-2H-1-benzopyran-4- one |
| 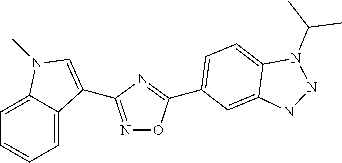 [SMILES: CC(C)N1NNc2cc(-c3nc(-c4cn(C)c5ccccc45)no3)ccc21] | 678 | 5-[3-(1-methyl-1H-indol-3-yl)- 1,2,4-oxadiazol-5-yl]-1-(propan- 2-yl)-1H-1,2,3-benzotriazole |
| 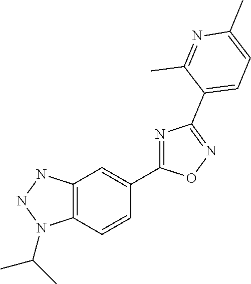 [SMILES: Cc1ccc(-c2noc(-c3ccc4c(c3)NNN4C(C)C)n2)c(C)n1] | 679 | 5-[3-(2,6-dimethylpyridin-3-yl)- 1,2,4-oxadiazol-5-yl]-1-(propan- 2-yl)-1H-1,2,3-benzotriazole |
| 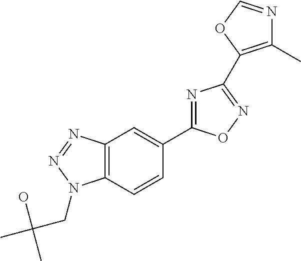 [SMILES: Cc1ncoc1-c1noc(-c2ccc3c(c2)nnn3CC(C)(C)O)n1] | 680 | 2-methyl-1-{5-[3-(4-methyl-1,3- oxazol-5-yl)-1,2,4-oxadiazol-5- yl]-1H-1,2,3-benzotriazol-1- yl}propan-2-ol |
| 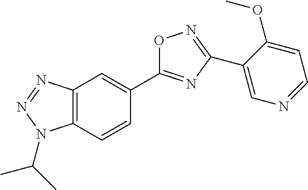 [SMILES: COc1ccncc1-c1noc(-c2ccc3c(c2)nnn3C(C)C)n1] | 681 | 5-(1-isopropyl-1H- benzo[d][1,2,3]triazol-5-yl)-3-(4- methoxypyridin-3-yl)-1,2,4- oxadiazole |
| 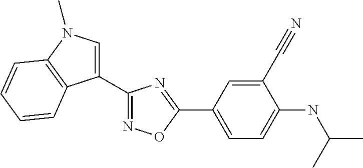 [SMILES: CC(C)Nc1ccc(-c2nc(-c3cn(C)c4ccccc34)no2)cc1C#N] | 682 | 5-[3-(1-methyl-1H-indol-3-yl)- 1,2,4-oxadiazol-5-yl]-2-[(propan- 2-yl)amino]benzonitrile |
| 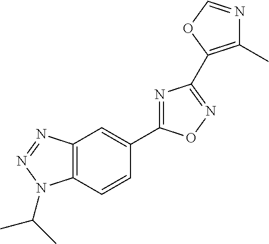 [SMILES: Cc1ncoc1-c1noc(-c2ccc3c(c2)nnn3C(C)C)n1] | 683 | 5-[3-(4-methyl-1,3-oxazol-5-yl)- 1,2,4-oxadiazol-5-yl]-1-(propan- 2-yl)-1H-1,2,3-benzotriazole |
| 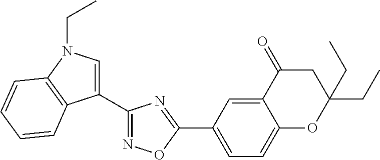 [SMILES: CCn1cc(-c2noc(-c3ccc4c(c3)C(=O)CC(CC)(CC)O4)n2)c2ccccc21] | 684 | 2,2-diethyl-6-[3-(1-ethyl-1H- indol-3-yl)-1,2,4-oxadiazol-5-yl]- 3,4-dihydro-2H-1-benzopyran-4- one |
| 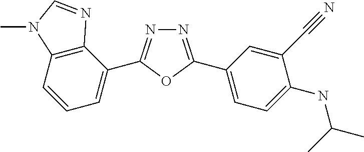 [SMILES: CC(C)Nc1ccc(-c2nnc(-c3cccc4c3ncn4C)o2)cc1C#N] | 685 | 5-[5-(1-methyl-1H-1,3- benzodiazol-4-yl)-1,3,4- oxadiazol-2-yl]-2-[(propan-2- yl)amino]benzonitrile |
| 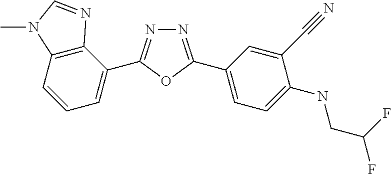 [SMILES: Cn1cnc2c(-c3nnc(-c4ccc(NCC(F)F)c(C#N)c4)o3)cccc21] | 686 | 2-[(2,2-difluoroethyl)amino]-5-[5- (1-methyl-1H-1,3-benzodiazol-4- yl)-1,3,4-oxadiazol-2- yl]benzonitrile |
| 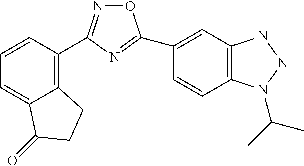 [SMILES: CC(C)N1NNc2cc(-c3nc(-c4cccc5c4CCC5=O)no3)ccc21] | 687 | 4-{5-[1-(propan-2-yl)-1H-1,2,3- benzotriazol-5-yl]-1,2,4- oxadiazol-3-yl}-2,3-dihydro-1H- inden-1-one |
| 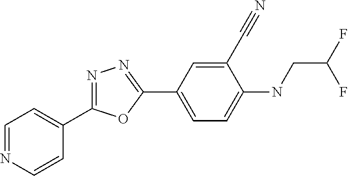 [SMILES: N#Cc1cc(-c2nnc(-c3ccncc3)o2)ccc1NCC(F)F] | 688 | 2-((2,2-difluoroethyl)amino)-5-(5- (pyridin-4-yl)-1,3,4-oxadiazol-2- yl)benzonitrile |
| 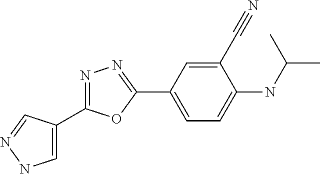 [SMILES: CC(C)Nc1ccc(-c2nnc(-c3cn[nH]c3)o2)cc1C#N] | 689 | 5-(5-(1H-pyrazol-4-yl)-1,3,4- oxadiazol-2-yl)-2- (isopropylamino)benzonitrile |
| 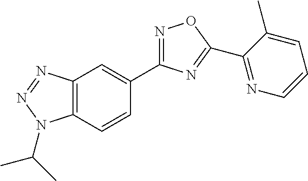 [SMILES: Cc1cccnc1-c1nc(-c2ccc3c(c2)nnn3C(C)C)no1] | 690 | 3-(1-isopropyl-1H- benzo[d][1,2,3]triazol-5-yl)-5-(3- methylpyridin-2-yl)-1,2,4- oxadiazole |
| 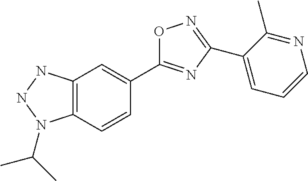 [SMILES: Cc1ncccc1-c1noc(-c2ccc3c(c2)NNN3C(C)C)n1] | 691 | 5-(1-isopropyl-1H- benzo[d][1,2,3]triazol-5-yl)-3-(2- methylpyridin-3-yl)-1,2,4- oxadiazole |
| 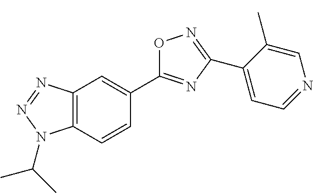 | 692 | 5-(1-isopropyl-1H- benzo[d][1,2,3]triazol-5-yl)-3-(3- methylpyridin-4-yl)-1,2,4- oxadiazole |
| 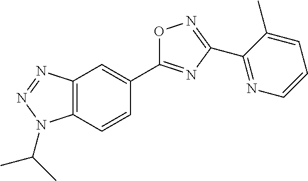 [SMILES: Cc1cccnc1-c1noc(-c2ccc3c(c2)nnn3C(C)C)n1] | 693 | 5-(1-isopropyl-1H- benzo[d][1,2,3]triazol-5-yl)-3-(3- methylpyridin-2-yl)-1,2,4- oxadiazole |
| 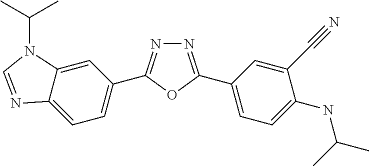 [SMILES: CC(C)Nc1ccc(-c2nnc(-c3ccc4ncn(C(C)C)c4c3)o2)cc1C#N] | 694 | 5-{5-[1-(propan-2-yl)-1H-1,3- benzodiazol-6-yl]-1,3,4- oxadiazol-2-yl}-2-[(propan-2- yl)amino]benzonitrile |
| 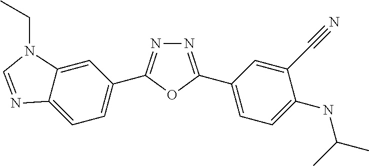 [SMILES: CCn1cnc2ccc(-c3nnc(-c4ccc(NC(C)C)c(C#N)c4)o3)cc21] | 695 | 5-[5-(1-ethyl-1H-1,3-benzodiazol- 6-yl)-1,3,4-oxadiazol-2-yl]-2- [(propan-2-yl)amino]benzonitrile |
| 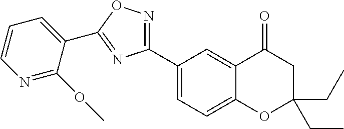 [SMILES: CCC1(CC)CC(=O)c2cc(-c3noc(-c4cccnc4OC)n3)ccc2O1] | 696 | 2,2-diethyl-6-[5-(2- methoxypyridin-3-yl)-1,2,4- oxadiazol-3-yl]-3,4-dihydro-2H- 1-benzopyran-4-one |
| 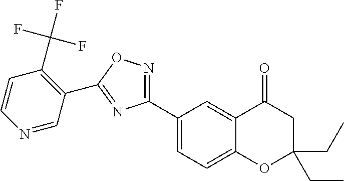 [SMILES: CCC1(CC)CC(=O)c2cc(-c3noc(-c4cnccc4C(F)(F)F)n3)ccc2O1] | 697 | 2,2-diethyl-6-{5-[4- (trifluoromethyl)pyridin-3-yl]- 1,2,4-oxadiazol-3-yl}-3,4- dihydro-2H-1-benzopyran-4-one |
| 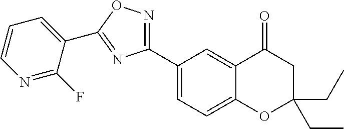 [SMILES: CCC1(CC)CC(=O)c2cc(-c3noc(-c4cccnc4F)n3)ccc2O1] | 698 | 2,2-diethyl-6-[5-(2-fluoropyridin- 3-yl)-1,2,4-oxadiazol-3-yl]-3,4- dihydro-2H-1-benzopyran-4-one |
| 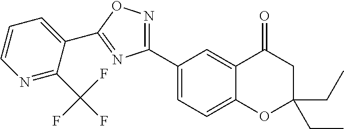 [SMILES: CCC1(CC)CC(=O)c2cc(-c3noc(-c4cccnc4C(F)(F)F)n3)ccc2O1] | 699 | 2,2-diethyl-6-{5-[2- (trifluoromethyl)pyridin-3-yl]- 1,2,4-oxadiazol-3-yl}-3,4- dihydro-2H-1-benzopyran-4-one |
| 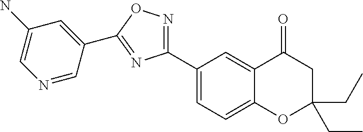 [SMILES: CCC1(CC)CC(=O)c2cc(-c3noc(-c4cncc(N)c4)n3)ccc2O1] | 700 | 6-[5-(5-aminopyridin-3-yl)-1,2,4- oxadiazol-3-yl]-2,2-diethyl-3,4- dihydro-2H-1-benzopyran-4-one |
| 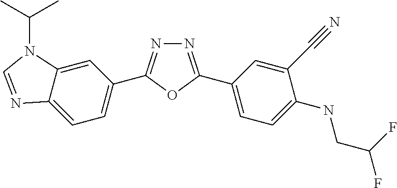 [SMILES: CC(C)n1cnc2ccc(-c3nnc(-c4ccc(NCC(F)F)c(C#N)c4)o3)cc21] | 701 | 2-[(2,2-difluoroethyl)amino]-5- {5-[1-(propan-2-yl)-1H-1,3- benzodiazol-6-yl]-1,3,4- oxadiazol-2-yl}benzonitrile |
| 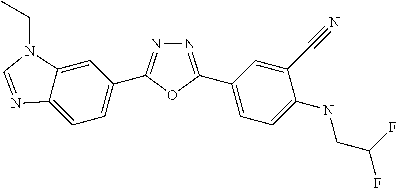 [SMILES: CCn1cnc2ccc(-c3nnc(-c4ccc(NCC(F)F)c(C#N)c4)o3)cc21] | 702 | 2-[(2,2-difluoroethyl)amino]-5-[5- (1-ethyl-1H-1,3-benzodiazol-6- yl)-1,3,4-oxadiazol-2- yl]benzonitrile |
| 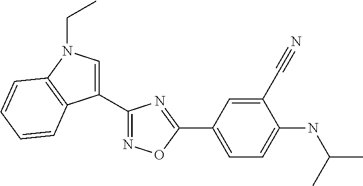 [SMILES: CCn1cc(-c2noc(-c3ccc(NC(C)C)c(C#N)c3)n2)c2ccccc21] | 703 | 5-[3-(1-ethyl-1H-indol-3-yl)- 1,2,4-oxadiazol-5-yl]-2-[(propan- 2-yl)amino]benzonitrile |
| 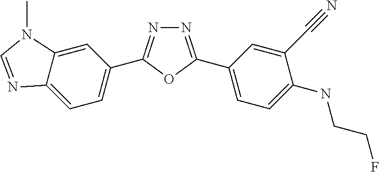 [SMILES: Cn1cnc2ccc(-c3nnc(-c4ccc(NCCF)c(C#N)c4)o3)cc21] | 704 | 2-[(2-fluoroethyl)amino]-5-[5-(1- methyl-1H-1,3-benzodiazol-6- yl)-1,3,4-oxadiazol-2- yl]benzonitrile |
| 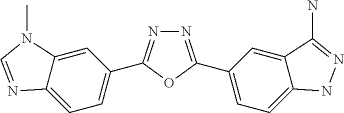 [SMILES: Cn1cnc2ccc(-c3nnc(-c4ccc5[nH]nc(N)c5c4)o3)cc21] | 705 | 5-[5-(1-methyl-1H-1,3- benzodiazol-6-yl)-1,3,4- oxadiazol-2-yl]-1H-indazol-3- amine |
| 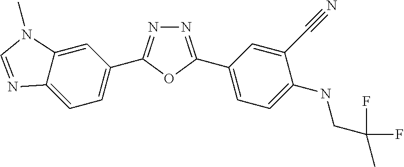 [SMILES: Cn1cnc2ccc(-c3nnc(-c4ccc(NCC(C)(F)F)c(C#N)c4)o3)cc21] | 706 | 2-[(2,2-difluoropropyl)amino]-5- [5-(1-methyl-1H-1,3- benzodiazol-6-yl)-1,3,4- oxadiazol-2-yl]benzonitrile |
| 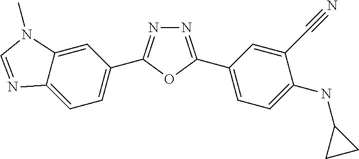 [SMILES: Cn1cnc2ccc(-c3nnc(-c4ccc(NC5CC5)c(C#N)c4)o3)cc21] | 707 | 2-(cyclopropylamino)-5-[5-(1- methyl-1H-1,3-benzodiazol-6- yl)-1,3,4-oxadiazol-2- yl]benzonitrile |
| 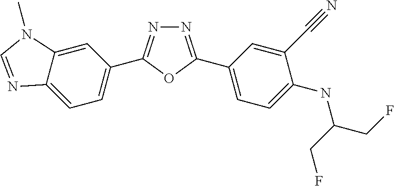 [SMILES: Cn1cnc2ccc(-c3nnc(-c4ccc(NC(CF)CF)c(C#N)c4)o3)cc21] | 708 | 2-[(1,3-difluoropropan-2- yl)amino]-5-[5-(1-methyl-1H- 1,3-benzodiazol-6-yl)-1,3,4- oxadiazol-2-yl]benzonitrile |
| 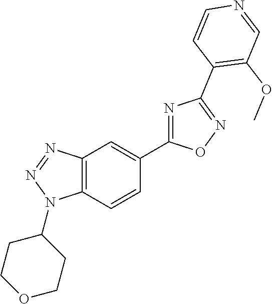 [SMILES: COc1cnccc1-c1noc(-c2ccc3c(c2)nnn3C2CCOCC2)n1] | 709 | 5-[3-(3-methoxypyridin-4-yl)- 1,2,4-oxadiazol-5-yl]-1-(oxan-4- yl)-1H-1,2,3-benzotriazole |
| 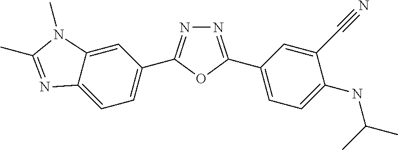 [SMILES: Cc1nc2ccc(-c3nnc(-c4ccc(NC(C)C)c(C#N)c4)o3)cc2n1C] | 710 | 5-[5-(1,2-dimethyl-1H-1,3- benzodiazol-6-yl)-1,3,4- oxadiazol-2-yl]-2-[(propan-2- yl)amino]benzonitrile |
| 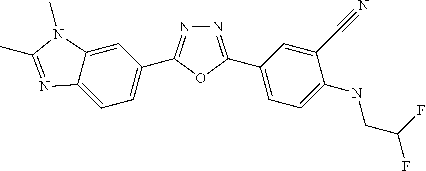 [SMILES: Cc1nc2ccc(-c3nnc(-c4ccc(NCC(F)F)c(C#N)c4)o3)cc2n1C] | 711 | 2-[(2,2-difluoroethyl)amino]-5-[5- (1,2-dimethyl-1H-1,3- benzodiazol-6-yl)-1,3,4- oxadiazol-2-yl]benzonitrile |
| 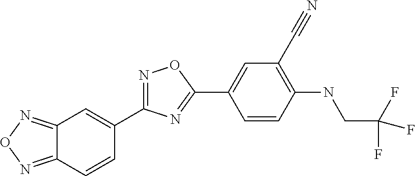 [SMILES: N#Cc1cc(-c2nc(-c3ccc4nonc4c3)no2)ccc1NCC(F)(F)F] | 712 | 5-[3-(2,1,3-benzoxadiazol-5-yl)- 1,2,4-oxadiazol-5-yl]-2-[(2,2,2- trifluoroethyl)amino]benzonitrile |
| 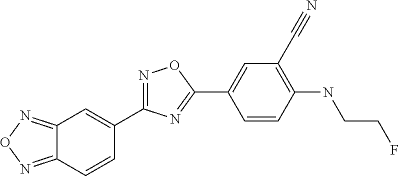 [SMILES: N#Cc1cc(-c2nc(-c3ccc4nonc4c3)no2)ccc1NCCF] | 713 | 5-[3-(2,1,3-benzoxadiazol-5-yl)- 1,2,4-oxadiazol-5-yl]-2-[(2- fluoroethyl)amino]benzonitrile |
| 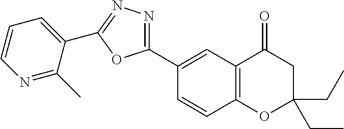 [SMILES: CCC1(CC)CC(=O)c2cc(-c3nnc(-c4cccnc4C)o3)ccc2O1] | 718 | 2,2-diethyl-6-(5-(2- methylpyridin-3-yl)-1,3,4- oxadiazol-2-yl)chroman-4-one |
| 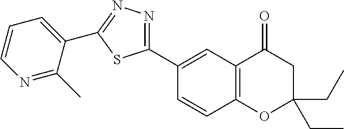 [SMILES: CCC1(CC)CC(=O)c2cc(-c3nnc(-c4cccnc4C)s3)ccc2O1] | 719 | 2,2-diethyl-6-(5-(2- methylpyridin-3-yl)-1,3,4- thiadiazol-2-yl)chroman-4-one |
| 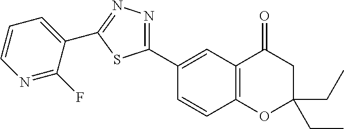 [SMILES: CCC1(CC)CC(=O)c2cc(-c3nnc(-c4cccnc4F)s3)ccc2O1] | 720 | 2,2-diethyl-6-(5-(2-fluoropyridin- 3-yl)-1,3,4-thiadiazol-2- yl)chroman-4-one |
| 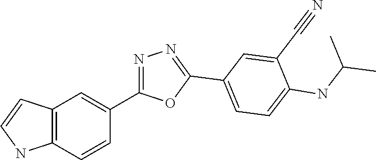 [SMILES: CC(C)Nc1ccc(-c2nnc(-c3ccc4[nH]ccc4c3)o2)cc1C#N] | 721 | 5-(5-(1H-indol-5-yl)-1,3,4- oxadiazol-2-yl)-2- (isopropylamino)benzonitrile |
| 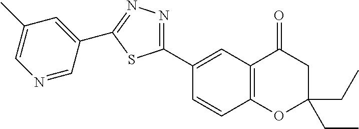 [SMILES: CCC1(CC)CC(=O)c2cc(-c3nnc(-c4cncc(C)c4)s3)ccc2O1] | 724 | 2,2-diethyl-6-(5-(5- methylpyridin-3-yl)-1,3,4- thiadiazol-2-yl)chroman-4-one |
| 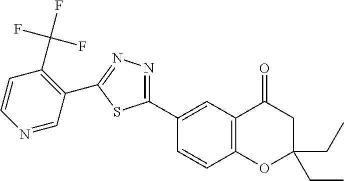 [SMILES: CCC1(CC)CC(=O)c2cc(-c3nnc(-c4cnccc4C(F)(F)F)s3)ccc2O1] | 725 | 2,2-diethyl-6-(5-(4- (trifluoromethyl)pyridin-3-yl)- 1,3,4-thiadiazol-2-yl)chroman-4- one |
| 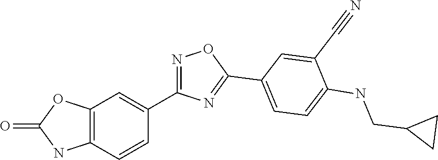 [SMILES: N#Cc1cc(-c2nc(-c3ccc4[nH]c(=O)oc4c3)no2)ccc1NCC1CC1] | 726 | 2-(cyclopropylmethylamino)-5- [3-(2-oxo-3H-1,3-benzoxazol-6- yl)-1,2,4-oxadiazol-5- yl]benzonitrile |
| 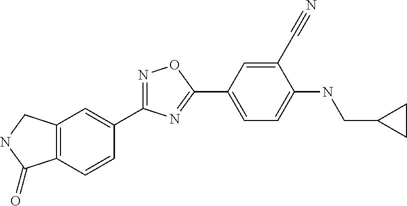 [SMILES: N#Cc1cc(-c2nc(-c3ccc4c(c3)CNC4=O)no2)ccc1NCC1CC1] | 727 | 2-((cyclopropylmethyl)amino)-5- (3-(1-oxoisoindolin-5-yl)-1,2,4- oxadiazol-5-yl)benzonitrile |
| 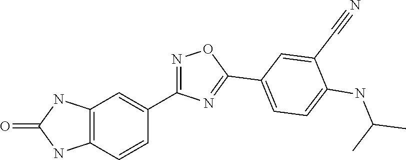 [SMILES: CC(C)Nc1ccc(-c2nc(-c3ccc4[nH]c(=O)[nH]c4c3)no2)cc1C#N] | 728 | 2-(isopropylamino)-5-(3-(2-oxo- 2,3-dihydro-1H- benzo[d]imidazol-5-yl)-1,2,4- oxadiazol-5-yl)benzonitrile |
| 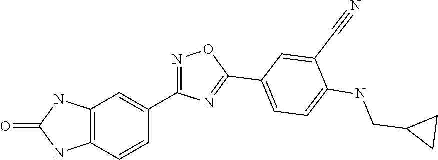 [SMILES: N#Cc1cc(-c2nc(-c3ccc4[nH]c(=O)[nH]c4c3)no2)ccc1NCC1CC1] | 729 | 2-((cyclopropylmethyl)amino)-5- (3-(2-oxo-2,3-dihydro-1H- benzo[d]imidazol-5-yl)-1,2,4- oxadiazol-5-yl)benzonitrile |
| 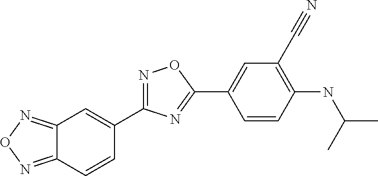 [SMILES: CC(C)Nc1ccc(-c2nc(-c3ccc4nonc4c3)no2)cc1C#N] | 730 | 5-[3-(2,1,3-benzoxadiazol-5-yl)- 1,2,4-oxadiazol-5-yl]-2-[(propan- 2-yl)amino]benzonitrile |
| 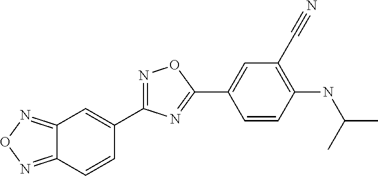 [SMILES: CC(C)Nc1ccc(-c2nc(-c3ccc4nonc4c3)no2)cc1C#N] | 730 | 5-[3-(2,1,3-benzoxadiazol-5-yl)- 1,2,4-oxadiazol-5-yl]-2-[(propan- 2-yl)amino]benzonitrile |
| 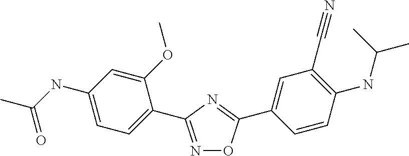 [SMILES: COc1cc(NC(C)=O)ccc1-c1noc(-c2ccc(NC(C)C)c(C#N)c2)n1] | 731 | N-[4-(5-{3-cyano-4-[(propan-2- yl)amino]phenyl}-1,2,4- oxadiazol-3-yl)-3- methoxyphenyl]acetamide |
| 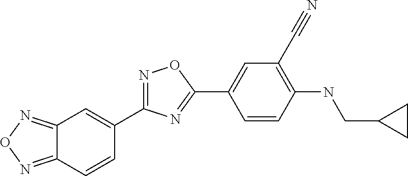 [SMILES: N#Cc1cc(-c2nc(-c3ccc4nonc4c3)no2)ccc1NCC1CC1] | 732 | 5-[3-(2,1,3-benzoxadiazol-5-yl)- 1,2,4-oxadiazol-5-yl]-2-[(2,2- difluoroethyl)amino]benzonitrile |
| 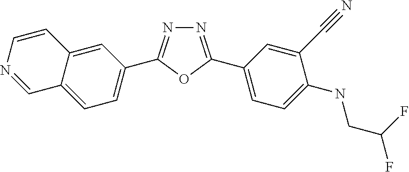 [SMILES: N#Cc1cc(-c2nnc(-c3ccc4cnccc4c3)o2)ccc1NCC(F)F] | 733 | 2-[(2,2-difluoroethyl)amino]-5-[5- (isoquinolin-6-yl)-1,3,4- oxadiazol-2-yl]benzonitrile |
| 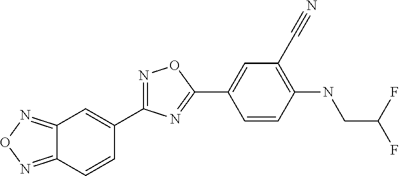 [SMILES: N#Cc1cc(-c2nc(-c3ccc4nonc4c3)no2)ccc1NCC(F)F] | 734 | 5-[3-(2,1,3-benzoxadiazol-5-yl)- 1,2,4-oxadiazol-5-yl]-2- [(cyclopropylmethyl)amino] benzonitrile |
| 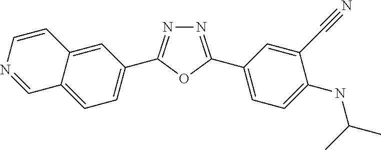 [SMILES: CC(C)Nc1ccc(-c2nnc(-c3ccc4cnccc4c3)o2)cc1C#N] | 735 | 5-[5-(isoquinolin-6-yl)-1,3,4- oxadiazol-2-yl]-2-[(propan-2- yl)amino]benzonitrile |
| 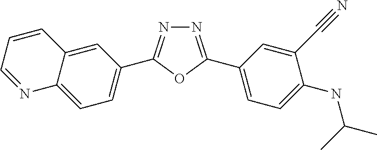 [SMILES: CC(C)Nc1ccc(-c2nnc(-c3ccc4ncccc4c3)o2)cc1C#N] | 736 | 2-[(propan-2-yl)amino]-5-[5- (quinolin-6-yl)-1,3,4-oxadiazol- 2-yl]benzonitrile |
| 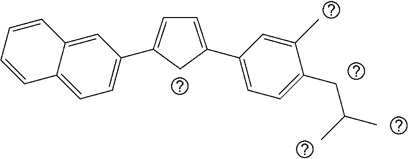 [SMILES: Cc1cc(C2=CC=C(c3ccc4ccccc4c3)C2)ccc1CC(C)C] | 736 | 2-[(propan-2-yl)amino]-5-[5- (quinolin-6-yl)-1,3,4-oxadiazol- 2-yl]benzonitrile |
| 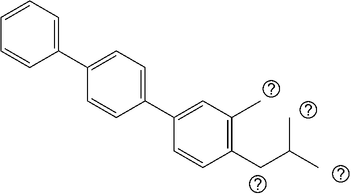 [SMILES: Cc1cc(-c2ccc(-c3ccccc3)cc2)ccc1CC(C)C] | 737 | 2-(isopropylamino)-5-(5-(pyridin- 3-yl)pyrimidin-2-yl)benzonitrile |
| 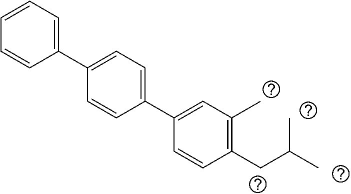 [SMILES: Cc1cc(-c2ccc(-c3ccccc3)cc2)ccc1CC(C)C] | 738 | 2-isopropoxy-5-(5-(pyridin-3- yl)pyrimidin-2-yl)benzonitrile |
| 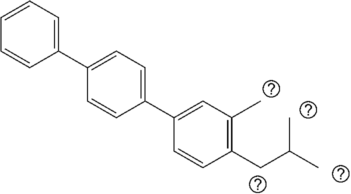 [SMILES: Cc1cc(-c2ccc(-c3ccccc3)cc2)ccc1CC(C)C] | 739 | 2-(isopropylamino)-5-(5-(pyridin- 4-yl)pyrimidin-2-yl)benzonitrile |
| 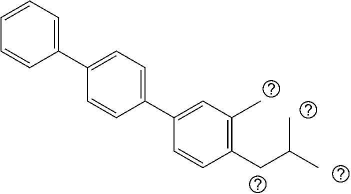 [SMILES: Cc1cc(-c2ccc(-c3ccccc3)cc2)ccc1CC(C)C] | 740 | 2-isopropoxy-5-(5-(pyridin-4- yl)pyrimidin-2-yl)benzonitrile |
| 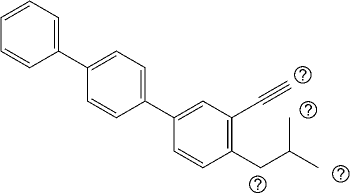 [SMILES: C#Cc1cc(-c2ccc(-c3ccccc3)cc2)ccc1CC(C)C] | 741 | 2-(isopropylamino)-5-(5-(pyridin- 2-yl)pyrimidin-2-yl)benzonitrile |
| 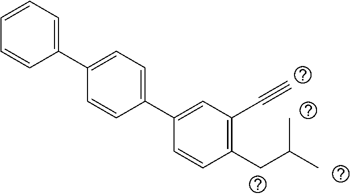 [SMILES: C#Cc1cc(-c2ccc(-c3ccccc3)cc2)ccc1CC(C)C] | 742 | 2-isopropoxy-5-(5-(pyridin-2- yl)pyrimidin-2-yl)benzonitrile |
| 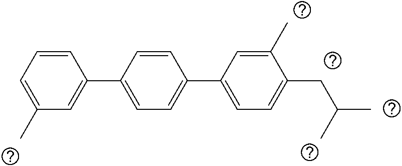 [SMILES: Cc1cccc(-c2ccc(-c3ccc(CC(C)C)c(C)c3)cc2)c1] | 743 | 5-(5-(2-hydroxypyridin-4- yl)pyrimidin-2-yl)-2- (isopropylamino)benzonitrile |
| 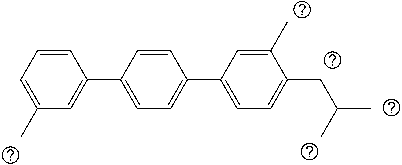 [SMILES: Cc1cccc(-c2ccc(-c3ccc(CC(C)C)c(C)c3)cc2)c1] | 744 | 5-(5-(2-hydroxypyridin-4- yl)pyrimidin-2-yl)-2- isopropoxybenzonitrile |
| 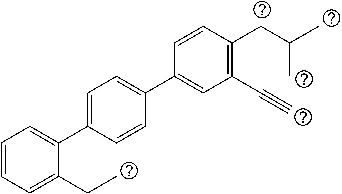 [SMILES: C#Cc1cc(-c2ccc(-c3ccccc3CC)cc2)ccc1CC(C)C] | 745 | 2-isopropoxy-5-(5-(2- methoxyphenyl)pyrimidin-2- yl)benzonitrile |
| 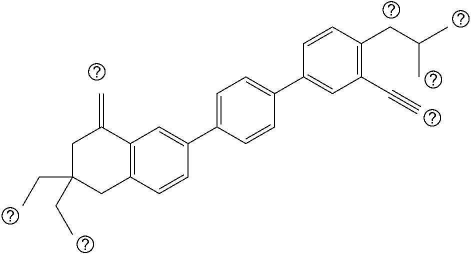 [SMILES: C#Cc1cc(-c2ccc(-c3ccc4c(c3)C(=C)CC(CC)(CC)C4)cc2)ccc1CC(C)C] | 746 | 5-(5-(2,2-diethyl-4-oxochroman- 6-yl)pyrimidin-2-yl)-2- (isopropylamino)benzonitrile |
| 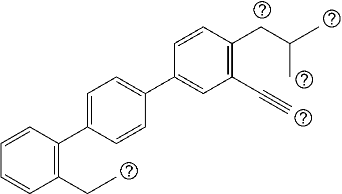 [SMILES: C#Cc1cc(-c2ccc(-c3ccccc3CC)cc2)ccc1CC(C)C] | 747 | 2-(isopropylamino)-5-(5-(2- methoxyphenyl)pyrimidin-2- yl)benzonitrile |
| 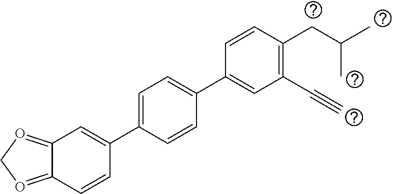 | 748 | 5-(5-(benzo[c][1,2,5]oxadiazol-5- yl)pyrimidin-2-yl)-2- isopropoxybenzonitrile |
| 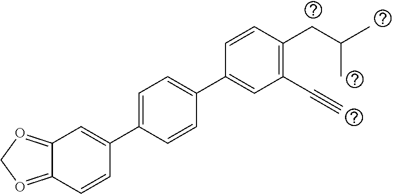 | 749 | 5-(5-(benzo[c][1,2,5]oxadiazol-5- yl)pyrimidin-2-yl)-2- (isopropylamino)benzonitrile |
| 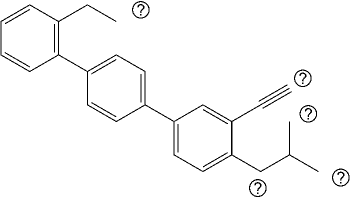 [SMILES: C#Cc1cc(-c2ccc(-c3ccccc3CC)cc2)ccc1CC(C)C] | 750 | N-isopropyl-4-(5-(2- methoxyphenyl)pyrimidin-2-yl)- 2-(trifluoromethyl)aniline |
| 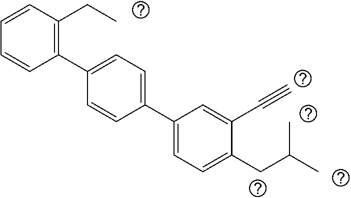 [SMILES: C#Cc1cc(-c2ccc(-c3ccccc3CC)cc2)ccc1CC(C)C] | 751 | 2-(4-isopropoxy-3- (trifluoromethyl)phenyl)-5-(2- methoxyphenyl)pyrimidine |
| 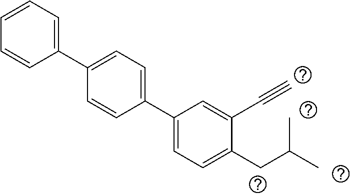 [SMILES: C#Cc1cc(-c2ccc(-c3ccccc3)cc2)ccc1CC(C)C] | 752 | 2-(4-isopropoxy-3- (trifluoromethyl)phenyl)-5- (pyridin-4-yl)pyrimidine |

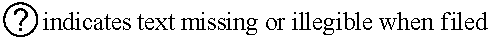

Although the compounds described herein may be shown with specific stereochemistries around certain atoms, such as cis or trans, the compounds can also be made in the opposite orientation or in a racemic mixture. Such isomers or racemic mixtures are encompassed by the present disclosure. Additionally, although the compounds are shown collectively in a table, any compounds, or a pharmaceutically acceptable salt thereof, can be chosen from the table and used in the embodiments provided for herein.

In some embodiments, pharmaceutical compositions comprising a compound or pharmaceutically salt thereof of any compound described herein for methods of treating or preventing a seizure, an epilepsy or an epilepsy-related syndrome in a subject as described herein are provided.

The compounds described herein can be made by can be made according to the methods described herein and in the examples. The methods described herein can be adapted based upon the compounds desired and described herein. In some embodiments, the method is made according to the following schemes, wherein Q and L are the substituents as shown and described herein and would be apparent to one of skill in the art based upon the present disclosure. In some embodiments, this method can be used to make one or more compounds as described herein and will be apparent to one of skill in the art which compounds can be made according to the methods described herein.

The conditions and temperatures can be varied, such as shown in the examples described herein. These schemes are non-limiting synthetic schemes and the synthetic routes can be modified as would be apparent to one of skill in the art reading the present specification. The compounds can also be prepared according to the schemes described in the Examples.

The compounds can be used to modulate the S1P1 receptor. Thus, in some embodiments, the compounds can be referred to as S1P1 receptor modulating compounds.

The compounds described herein can be administered in any conventional manner by any route where they are active. Administration can be systemic, topical, or oral. For example, administration can be, but is not limited to, parenteral, subcutaneous, intravenous, intramuscular, intraperitoneal, transdermal, oral, buccal, sublingual, or ocular routes, or intravaginal, by inhalation, by depot injections, or by implants. The mode of administration can depend on the conditions or disease to be targeted or treated. The selection of the specific route of administration can be selected or adjusted by the clinician according to methods known to the clinician to obtain the desired clinical response.

In some embodiments, it may be desirable to administer one or more compounds, or a pharmaceutically acceptable salt thereof, locally to an area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, wherein the implant is of a porous, non-porous, or gelatinous material, including membranes, such as sialastic membranes, or fibers.

The compounds described herein can be administered either alone or in combination (concurrently or serially) with other pharmaceuticals. For example, the compounds can be administered in combination with other anti-epileptic drugs and the like. Examples of other pharmaceuticals or medicaments are known to one of skill in the art and include, but are not limited to those described herein.

The means and methods for administration are known in the art and an artisan can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician). The standard dosing for protamine can be used and adjusted (i.e., increased or decreased) depending upon the factors described above. The selection of the specific dose regimen can be selected or adjusted or titrated by the clinician according to methods known to the clinician to obtain the desired clinical response.

The amount of a compound described herein that will be effective in the treatment and/or prevention of a particular disease, condition, or disorder will depend on the nature and extent of the disease, condition, or disorder, and can be determined by standard clinical techniques. In addition, in vitro or in vivo assays may optionally be employed to help identify optimal dosage ranges. The precise dose to be employed in the compositions will also depend on the route of administration, and the seriousness of the disorder, and should be decided according to the judgment of the practitioner and each patient's circumstances. However, a suitable dosage range for oral administration is, generally, from about 0.001 milligram to about 200 milligrams per kilogram body weight, from about 0.01 milligram to about 100 milligrams per kilogram body weight, from about 0.01 milligram to about 70 milligrams per kilogram body weight, from about 0.1 milligram to about 50 milligrams per kilogram body weight, from 0.5 milligram to about 20 milligrams per kilogram body weight, or from about 1 milligram to about 10 milligrams per kilogram body weight. In some embodiments, the oral dose is about 5 milligrams per kilogram body weight.

In some embodiments, suitable dosage ranges for intravenous (i.v.) administration are from about 0.01 mg to about 500 mg per kg body weight, from about 0.1 mg to about 100 mg per kg body weight, from about 1 mg to about 50 mg per kg body weight, or from about 10 mg to about 35 mg per kg body weight. Suitable dosage ranges for other modes of administration can be calculated based on the forgoing dosages as known by those skilled in the art. For example, recommended dosages for intranasal, transmucosal, intradermal, intramuscular, intraperitoneal, subcutaneous, epidural, sublingual, intracerebral, intravaginal, transdermal administration or administration by inhalation are in the range of from about 0.001 mg to about 200 mg per kg of body weight, from about 0.01 mg to about 100 mg per kg of body weight, from about 0.1 mg to about 50 mg per kg of body weight, or from about 1 mg to about 20 mg per kg of body weight. Effective doses may be extrapolated from dose-response curves derived from in vitro or animal model test systems. Such animal models and systems are well known in the art.

The compounds described herein can be formulated for parenteral administration by injection, such as by bolus injection or continuous infusion. The compounds can be administered by continuous infusion subcutaneously over a period of about 15 minutes to about 24 hours. Formulations for injection can be presented in unit dosage form, such as in ampoules or in multi-dose containers, with an optionally added preservative. The compositions can take such forms as suspensions, solutions or emulsions in oily or aqueous vehicles, and can contain formulatory agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

For oral administration, the compounds described herein can be formulated by combining the compounds with pharmaceutically acceptable carriers well known in the art. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations, which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

For buccal administration, the compositions can take the form of, such as, tablets or lozenges formulated in a conventional manner.

For administration by inhalation, the compounds described herein can be delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

The compounds described herein can also be formulated in rectal compositions such as suppositories or retention enemas, such as containing conventional suppository bases such as cocoa butter or other glycerides. The compounds described herein can also be formulated in vaginal compositions such as vaginal creams, suppositories, pessaries, vaginal rings, and intrauterine devices.

In transdermal administration, the compounds can be applied to a plaster, or can be applied by transdermal, therapeutic systems that are consequently supplied to the organism. In some embodiments, the compounds are present in creams, solutions, powders, fluid emulsions, fluid suspensions, semi-solids, ointments, pastes, gels, jellies, and foams, or in patches containing any of the same.

The compounds described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In some embodiments, the compounds can be delivered in a controlled release system. In one embodiment, a pump may be used (see Langer, supra; Sefton, CRC Crit. Ref. Biomed. Eng., 1987, 14, 201; Buchwald et al., Surgery, 1980, 88, 507 Saudek et al., N. Engl. J. Med., 1989, 321, 574). In some embodiments, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger et al., J. Macromol. Sci. Rev. Macromol. Chem., 1983, 23, 61; see, also Levy et al., Science, 1985, 228, 190; During et al., Ann. Neurol., 1989, 25, 351; Howard et al., J. Neurosurg., 1989, 71, 105). In yet another embodiment, a controlled-release system can be placed in proximity of the target of the compounds described herein, such as the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, Science, 1990, 249, 1527-1533) may be used.

It is also known in the art that the compounds can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol).

In some embodiments, the compounds described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

Suitable compositions include, but are not limited to, oral non-absorbed compositions. Suitable compositions also include, but are not limited to saline, water, cyclodextrin solutions, and buffered solutions of pH 3-9.

The compounds described herein, or pharmaceutically acceptable salts thereof, can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, and 1.2% saline, and any combination thereof. In some embodiments, excipient is chosen from propylene glycol, purified water, and glycerin.

In some embodiments, the formulation can be lyophilized to a solid and reconstituted with, for example, water prior to use.

When administered to a mammal (e.g., to an animal for veterinary use or to a human for clinical use) the compounds can be administered in isolated form.

When administered to a human, the compounds can be sterile. Water is a suitable carrier when the compound of Formula I is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions described herein can take the form of a solution, suspension, emulsion, tablet, pill, pellet, capsule, capsule containing a liquid, powder, sustained-release formulation, suppository, aerosol, spray, or any other form suitable for use. Examples of suitable pharmaceutical carriers are described in Remington's Pharmaceutical Sciences, A. R. Gennaro (Editor) Mack Publishing Co.

In some embodiments, the compounds are formulated in accordance with routine procedures as a pharmaceutical composition adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, a composition is in the form of a liquid wherein the active agent (i.e., one of the facially amphiphilic polymers or oligomers disclosed herein) is present in solution, in suspension, as an emulsion, or as a solution/suspension. In some embodiments, the liquid composition is in the form of a gel. In other embodiments, the liquid composition is aqueous. In other embodiments, the composition is in the form of an ointment.

In some embodiments, the composition is in the form of a solid article. For example, in some embodiments, the ophthalmic composition is a solid article that can be inserted in a suitable location in the eye, such as between the eye and eyelid or in the conjunctival sac, where it releases the active agent as described, for example, U.S. Pat. Nos. 3,863,633; 3,867,519; 3,868,445; 3,960,150; 3,963,025; 4,186,184; 4,303,637; 5,443,505; and 5,869,079. Release from such an article is usually to the cornea, either via the lacrimal fluid that bathes the surface of the cornea, or directly to the cornea itself, with which the solid article is generally in intimate contact. Solid articles suitable for implantation in the eye in such fashion are generally composed primarily of polymers and can be bioerodible or non-bioerodible. Bioerodible polymers that can be used in the preparation of ocular implants carrying one or more of compounds include, but are not limited to, aliphatic polyesters such as polymers and copolymers of poly(glycolide), poly(lactide), poly(epsilon-caprolactone), poly-(hydroxybutyrate) and poly(hydroxyvalerate), polyamino acids, polyorthoesters, polyanhydrides, aliphatic polycarbonates and polyether lactones. Suitable non-bioerodible polymers include silicone elastomers.

The compositions described herein can contain preservatives. Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

Optionally one or more stabilizers can be included in the compositions to enhance chemical stability where required. Suitable stabilizers include, but are not limited to, chelating agents or complexing agents, such as, for example, the calcium complexing agent ethylene diamine tetraacetic acid (EDTA). For example, an appropriate amount of EDTA or a salt thereof, e.g., the disodium salt, can be included in the composition to complex excess calcium ions and prevent gel formation during storage. EDTA or a salt thereof can suitably be included in an amount of about 0.01% to about 0.5%. In those embodiments containing a preservative other than EDTA, the EDTA or a salt thereof, more particularly disodium EDTA, can be present in an amount of about 0.025% to about 0.1% by weight.

One or more antioxidants can also be included in the compositions. Suitable antioxidants include, but are not limited to, ascorbic acid, sodium metabisulfite, sodium bisulfite, acetylcysteine, polyquaternium-1, benzalkonium chloride, thimerosal, chlorobutanol, methyl paraben, propyl paraben, phenylethyl alcohol, edetate disodium, sorbic acid, or other agents know to those of skill in the art. Such preservatives are typically employed at a level of from about 0.001% to about 1.0% by weight.

In some embodiments, the compounds are solubilized at least in part by an acceptable solubilizing agent. Certain acceptable nonionic surfactants, for example polysorbate 80, can be useful as solubilizing agents, as can ophthalmically acceptable glycols, polyglycols, e.g., polyethylene glycol 400 (PEG-400), and glycol ethers.

Suitable solubilizing agents for solution and solution/suspension compositions are cyclodextrins. Suitable cyclodextrins can be chosen from α-cyclodextrin, β-cyclodextrin, γ-cyclodextrin, alkylcyclodextrins (e.g., methyl-β-cyclodextrin, dimethyl-β-cyclodextrin, diethyl-β-cyclodextrin), hydroxyalkylcyclodextrins (e.g., hydroxyethyl-β-cyclodextrin, hydroxypropyl-β-cyclodextrin), carboxy-alkylcyclodextrins (e.g., carboxymethyl-β-cyclodextrin), sulfoalkylether cyclodextrins (e.g., sulfobutylether-β-cyclodextrin), and the like. Ophthalmic applications of cyclodextrins have been reviewed in Rajewski et al., Journal of Pharmaceutical Sciences, 1996, 85, 1155-1159.

In some embodiments, the composition optionally contains a suspending agent. For example, in those embodiments in which the composition is an aqueous suspension or solution/suspension, the composition can contain one or more polymers as suspending agents. Useful polymers include, but are not limited to, water-soluble polymers such as cellulosic polymers, for example, hydroxypropyl methylcellulose, and water-insoluble polymers such as cross-linked carboxyl-containing polymers.

One or more acceptable pH adjusting agents and/or buffering agents can be included in the compositions, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

One or more acceptable salts can be included in the compositions in an amount required to bring osmolality of the composition into an acceptable range. Such salts include, but are not limited to, those having sodium, potassium or ammonium cations and chloride, citrate, ascorbate, borate, phosphate, bicarbonate, sulfate, thiosulfate or bisulfite anions. In some embodiments, salts include sodium chloride, potassium chloride, sodium thiosulfate, sodium bisulfite and ammonium sulfate. In some embodiments, the salt is sodium chloride.

Optionally one or more acceptable surfactants, preferably nonionic surfactants, or co-solvents can be included in the compositions to enhance solubility of the components of the compositions or to impart physical stability, or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40; polysorbate 20, 60 and 80; polyoxyethylene/polyoxypropylene surfactants (e.g., Pluronic® F-68, F84 and P-103); cyclodextrin; or other agents known to those of skill in the art. Typically, such co-solvents or surfactants are employed in the compositions at a level of from about 0.01% to about 2% by weight.

In some embodiments, pharmaceutical packs or kits comprising one or more containers filled with one or more compounds described herein are provided. Optionally associated with such container(s) can be a notice in the form prescribed by a governmental agency regulating the manufacture, use or sale of pharmaceuticals or biological products, which notice reflects approval by the agency of manufacture, use or sale for human administration for treating a condition, disease, or disorder described herein. In some embodiments, the kit contains more than one compound described herein. In some embodiments, the kit comprises a compound described herein in a single injectable dosage form, such as a single dose within an injectable device such as a syringe with a needle.

Modulation of the S1P1 receptor has been found to be a target for the treatment of certain disorders.

In some embodiments, the compounds, or pharmaceutically acceptable salts thereof, are administered to the subject for any condition or indication provided for herein without causing significant lymphopenia or immunosuppression. In some embodiments, the methods are performed without causing lymphopenia or immunosuppression.

In some embodiments, the methods as described herein comprise administering to the subject one or more compounds described herein or a pharmaceutically acceptable salt thereof, or a pharmaceutical composition of the same. In some embodiments, the subject is a subject in need of such treatment. As described herein, in some embodiments, the subject is a mammal, such as, but not limited to, a human.

In some embodiments, also provided are one or more compounds described above, or a pharmaceutically acceptable salt thereof, or a pharmaceutical composition comprising one or more compounds described above, for use in the manufacture of a medicament for the treatment and/or prevention of a seizure, epilepsy and/or epilepsy related syndrome, including, but not limited to the conditions described herein, in a subject, such as those described herein. In some embodiments, the subject is a subject in need thereof.

In some embodiments, also provided are one or more compounds described above, or a pharmaceutically acceptable salt thereof, or a pharmaceutical composition comprising one or more compounds described above, for use in the manufacture of a medicament for the treatment of a seizure, epilepsy, and/or epilepsy related syndrome, including, but not limited to the conditions described herein, in a subject, such as those described herein. In some embodiments, the subject is a subject in need thereof.

In some embodiments, also provided are one or more compounds described above, or a pharmaceutically acceptable salt thereof, or a pharmaceutical composition comprising one or more compounds described above, for use in the manufacture of a medicament for the prevention of a seizure, epilepsy, and/or epilepsy related syndrome, including, but not limited to the conditions described herein, in a subject, such as those described herein. In some embodiments, the subject is a subject in need thereof.

In some embodiments, also provided are one or more compounds described above, or a pharmaceutically acceptable salt thereof, or a pharmaceutical composition comprising one or more compounds described above, for use in the treatment and/or prevention of a seizure, epilepsy, and/or epilepsy related syndrome, including, but not limited to the conditions described herein, in a subject, such as those described herein. In some embodiments, the subject is a subject in need thereof.

In some embodiments, also provided are one or more compounds described above, or a pharmaceutically acceptable salt thereof, or a pharmaceutical composition comprising one or more compounds described above, for use in the treatment of a seizure, epilepsy, and/or epilepsy related syndrome, including, but not limited to the conditions described herein, in a subject, such as those described herein. In some embodiments, the subject is a subject in need thereof.

In some embodiments, also provided are one or more compounds described above, or a pharmaceutically acceptable salt thereof, or a pharmaceutical composition comprising one or more compounds described above, for use in the prevention of a seizure, epilepsy, and/or epilepsy related syndrome, including, but not limited to the conditions described herein, in a subject, such as those described herein. In some embodiments, the subject is a subject in need thereof.

In some embodiments, also provided are one or more compounds described above, or a pharmaceutically acceptable salt thereof, or a pharmaceutical composition comprising one or more compounds described above, for use in the treatment and/or prevention of a seizure, epilepsy, and/or epilepsy related syndrome, including, but not limited to the conditions described herein, in a subject, such as those described herein. In some embodiments, the subject is a subject in need thereof.

The present embodiments also provide the use of one or more compounds described above, or a pharmaceutically acceptable salt thereof, or a pharmaceutical composition comprising one or more compounds described above, in the modulation of a S1P1 receptor activity, such as the presence on the surface of the cell. In some embodiments, the compounds, pharmaceutically acceptable salts thereof, or pharmaceutical compositions of the same modulate the internalization, trafficking, and/or degradation of the S1P1 receptor. In some embodiments, the compounds, pharmaceutically acceptable salts thereof, or pharmaceutical compositions of the same modulate the G-protein modulated pathway of the S1P1 receptor.

The present embodiments also provide the use of one or more compounds described above, or a pharmaceutically acceptable salt thereof, or a pharmaceutical composition comprising one or more compounds described above, in the modulation of a S1P1 receptor activity, such as the presence on the surface of the cell. In some embodiments, the compounds, pharmaceutically acceptable salts thereof, or pharmaceutical compositions of the same modulate the internalization, trafficking, and/or degradation of the S1P1 receptor. In some embodiments, the compounds, pharmaceutically acceptable salts thereof, or pharmaceutical compositions of the same modulate the G-protein modulated pathway of the S1P1 receptor.

As used herein, “modulation” can refer to either inhibition or enhancement of a specific activity. For example, the modulation of the S1P1 receptor can refer to the inhibition and/or activation of the G-protein mediated pathway of the S1P1 receptor. In some embodiments, the modulation refers to the inhibition or activation of the P-arrestin mediated pathway of the S1P1 receptor. In some embodiments, the modulation refers to the inhibition or activation of the internalization of the S1P1 receptor. In some embodiments, the modulation refers to the inhibition or activation of any cell signaling pathway, or intracellular and/or extracellular entity that are directly or indirectly modulated by S1P1 receptors. The activity of a S1P1 receptor can be measured by any method including but not limited to the methods described herein.

The compounds described herein can be agonists, or agonist-like, or antagonists, or antagonist-like, of the S1P1 receptor. The ability of the compounds to stimulate or inhibit S1P1 receptor signaling may be measured using any assay known in the art used to detect S1P1 receptor mediated signaling or S1P1 receptor activity, or the absence of such signaling/activity. “S1P1 receptor activity” refers to the ability of a S1P1 receptor to transduce a signal. Such activity can be measured, e.g., in a heterologous cell, by coupling an S1P1 receptor (or a chimeric S1P1 receptor) to a downstream effector such as adenylate cyclase.

A “natural ligand-induced activity” as used herein, refers to activation of the S1P1 receptor by an endogenous ligand of the S1P1 receptor. Activity can be assessed using any number of endpoints to measure S1P1 receptor activity.

Generally, assays for testing compounds that modulate S1P1 receptor-mediated signal transduction include the determination of any parameter that is indirectly or directly under the influence of a S1P1 receptor, e.g., a functional, physical, or chemical effect.

Samples or assays comprising S1P1 receptors that are treated with a potential activator, inhibitor, or modulator are compared to control samples without the inhibitor, activator, or modulator to examine the extent of inhibition. Control samples (untreated with inhibitors) are assigned a relative S1P1 receptor activity value of 100%. Inhibition of a S1P1 receptor is achieved when the S1P1 receptor activity value relative to the control is about 80%, 50%, or 25%. Activation of a S1P1 receptor is achieved when the S1P1 receptor activity value relative to the control (untreated with activators) is 110%, 150%, or 200-500% (i.e., two to five fold higher relative to the control), or 1000-3000% or higher. For example, in some embodiments, assays comprising S1P1 receptors that are treated with a potential activator, inhibitor, or modulator are utilized to measure the functional capability of test compounds to either inhibit the activity of a known S1P1 receptor agonist, or to activate cell signaling pathways measured in the assay.

Inhibition of a S1P1 receptor is achieved when the measured activity value elicited by a known S1P1 receptor agonist such as S1P (endogenous ligand), or fingolimod, is blocked by the compound tested. Activation of S1P1 receptors by test compounds is achieved when the S1P1 receptors activation by the compounds tested is 50% or higher relative to the full efficacy of a known agonist (SIP, fingolimod).

The effects of the compounds upon the function of an S1P1 receptor can be measured by examining any of the parameters described above. Any suitable physiological change that affects S1P1 receptor activity can be used to assess the influence of a compound on the S1P1 receptors and natural ligand-mediated S1P1 receptor activity. When the functional consequences are determined using intact cells or animals, one can also measure a variety of effects such as changes in intracellular second messengers such as cAMP.

Modulators of S1P1 receptor activity can be tested using S1P1 receptor polypeptides as described herein, either recombinant or naturally occurring. The protein can be isolated, expressed in a cell, expressed in a membrane derived from a cell, expressed in tissue or in an animal. For example, neuronal cells, cells of the immune system, transformed cells, or membranes can be used to test the S1P1 receptor polypeptides described herein. Modulation is tested using one of the in vitro or in vivo assays described herein. Signal transduction and cellular trafficking can also be examined in vitro with soluble or solid state reactions, using a chimeric molecule such as an extracellular domain of a receptor covalently linked to a heterologous signal transduction domain, or a heterologous extracellular domain covalently linked to the transmembrane and or cytoplasmic domain of a receptor. Furthermore, ligand-binding domains of the protein of interest can be used in vitro in soluble or solid state reactions to assay for ligand binding.

Ligand binding to an S1P1 receptor, a domain, or chimeric protein can be tested in a number of formats. Binding can be performed in solution, in a bilayer membrane, attached to a solid phase, in a lipid monolayer, or in vesicles. For example, in an assay, the binding of the natural ligand to its receptor is measured in the presence of a candidate modulator, such as the compound described herein. Alternatively, the binding of the candidate modulator may be measured in the presence of the natural ligand. Often, competitive assays that measure the ability of a compound to compete with binding of the natural ligand to the receptor are used. Binding can be tested by measuring, e.g., changes in spectroscopic characteristics (e.g., fluorescence, absorbance, refractive index), hydrodynamic (e.g., shape) changes, or changes in chromatographic or solubility properties.

Another technology that can be used to evaluate S1P1 receptor-protein interactions in living cells involves bioluminescence resonance energy transfer (BRET). A detailed discussion regarding BRET can be found in Kroeger et al., J. Biol. Chem., 276(16):12736 43 (2001).

After the receptor is expressed in a cell, the cells can be grown in appropriate media in the appropriate cell plate. The cells can be plated, for example at 5000-10000 cells per well in a 384 well plate. In some embodiments, the cells are plated at about 1000, 2000, 3000, 4000, 5000, 6000, 7000, 8000, 9000, or 10000 cells/per well. The plates can have any number of wells and the number of cells can be modified accordingly.

Any medicament having utility in an application described herein can be used in co-therapy, co-administration or co-formulation with a composition as described above. Therefore, the compounds described herein can be administered either before, concurrently with, or after such therapeutics are administered to a subject.

The additional medicament can be administered in co-therapy (including co-formulation) with the one or more of the compounds described herein.

In some embodiments, the response of the disease or disorder to the treatment is monitored and the treatment regimen is adjusted if necessary in light of such monitoring.

Frequency of administration is typically such that the dosing interval, for example, the period of time between one dose and the next, during waking hours is from about 2 to about 12 hours, from about 3 to about 8 hours, or from about 4 to about 6 hours. It will be understood by those of skill in the art that an appropriate dosing interval is dependent to some degree on the length of time for which the selected composition is capable of maintaining a concentration of the compound(s) in the subject and/or in the target tissue (e.g., above the EC₅₀ (the minimum concentration of the compound which modulates the receptor's activity by 90%). Ideally, the concentration remains above the EC₅₀ for at least 100% of the dosing interval. Where this is not achievable it is desired that the concentration should remain above the EC₅₀ for at least about 60% of the dosing interval or should remain above the EC₅₀ for at least about 40% of the dosing interval.

The present disclosure also provides the following non-limiting embodiments:

In order that the embodiments disclosed herein may be more efficiently understood, examples are provided below. It should be understood that these examples are for illustrative purposes only and are not to be construed as limiting the embodiments in any manner.

Throughout these examples, there may be molecular cloning reactions, and other standard recombinant DNA techniques described and these were carried out according to methods described in Maniatis et al., Molecular Cloning—A Laboratory Manual, 2nd ed., Cold Spring Harbor Press (1989), using commercially available reagents, except where otherwise noted.

The following examples are illustrative, but not limiting, of the methods and compositions described herein. Other suitable modifications and adaptations of the variety of conditions and parameters normally encountered in therapy, synthesis, and other embodiments disclosed herein are within the spirit and scope of the embodiments.

### EXAMPLES

### Example 1: Synthesis of Compounds

Certain synthetic schemes, both general and specific, are provided herein. The compounds disclosed herein can be made according to the methods described herein or intermediates that lead to the compounds disclosed herein can be made according to the methods described herein. The substitutions can be varied according to the compound or intermediate being made based upon the following examples and other modifications known to one of skill in the art.

The following compounds were prepared according to the following examples or the examples were varied according to one of skill in the art to prepare the compounds.

| | Com- | | | |
|---|---|---|---|---|
| | pound | Chemical | Exact | Actual |
| Structure | Number | Name | Mass | Peak |
| 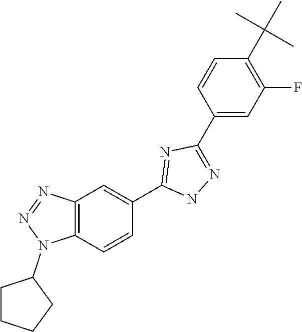 [SMILES: CC(C)(C)c1ccc(-c2n[nH]c(-c3ccc4c(c3)nnn4C3CCCC3)n2)cc1F] | 1 | 1-cyclopentyl-5-{3-[3- fluoro-4-(trifluoro- methyl)phenyl]-1,2,4- oxadiazol-5-yl}-1H-1,2,3- benzotriazole | 417.37 | 418.1 |
| 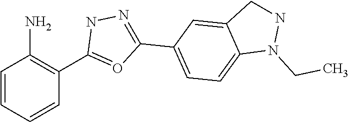 | 2 | 5-(2-bromophenyl)-3-(1- ethyl-1H-benzo[d][1,2,3] triazol-5-yl)-1,2,4- oxadiazole | 370.21 | 371 |
| 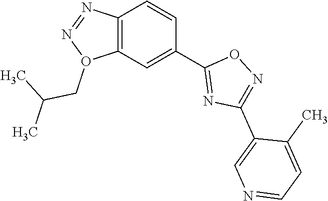 | 3 | 1-(2-methylpropyl)-6-[3-(4- methylpyridin-3-yl)-1,2,4- oxadiazol-5-yl]-1H-1,2,3- benzotriazole | 334.38 | 355.2 |
| 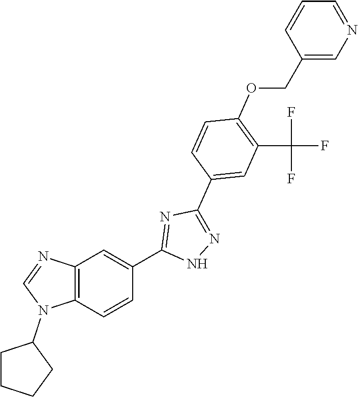 [SMILES: FC(F)(F)c1cc(-c2n[nH]c(-c3ccc4c(c3)ncn4C3CCCC3)n2)ccc1OCc1cccnc1] | 4 | 1-cyclopentyl-5-{3-[4- (pyridin-3-ylmethoxy)-3- (trifluoromethyl)phenyl]- 1,2,4-oxadiazol-5-yl}-1H- 1,2,3-benzotriazole | 506.49 | 507.2 |
| 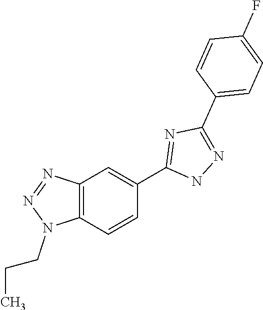 [SMILES: CCCn1nnc2cc(-c3nc(-c4ccc(F)cc4)n[nH]3)ccc21] | 5 | 5-[3-(4-fluorophenyl)- 1,2,4-oxadiazol-5-yl]-1- propyl-1H-1,2,3- benzotriazole | 323.33 | 324.2 |
| 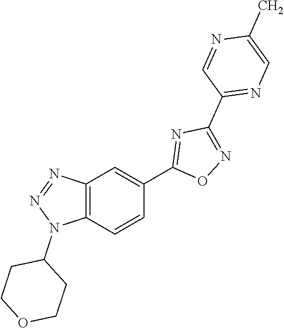 [SMILES: Cc1cnc(-c2noc(-c3ccc4c(c3)nnn4C3CCOCC3)n2)cn1] | 6 | 5-[3-(5-methylpyrazin- 2-yl)-1,2,4-oxadiazol-5- yl]-1-(oxan-4-yl)-1H- 1,2,3-benzotriazole | 363.38 | 364.1 |
| 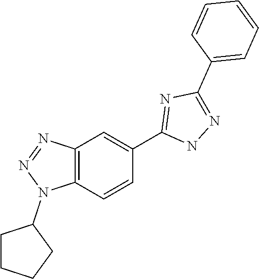 [SMILES: c1ccc(-c2n[nH]c(-c3ccc4c(c3)nnn4C3CCCC3)n2)cc1] | 7 | 1-cyclopentyl-5-(3-phenyl- 1,2,4-oxadiazol-5-yl)- 1H-1,2,3-benzotriazole | 331.38 | 332.1 |
| 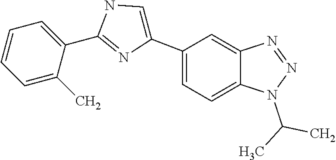 [SMILES: Cc1ccccc1-c1nc(-c2ccc3c(c2)nnn3C(C)C)c[nH]1] | 8 | 3-(1-isopropyl-1H- benzo[d][1,2,3]triazol-5- yl)-5-(3-methylpyridin- 4-yl)-1,2,4-oxadiazole | 320.36 | 321 |
| 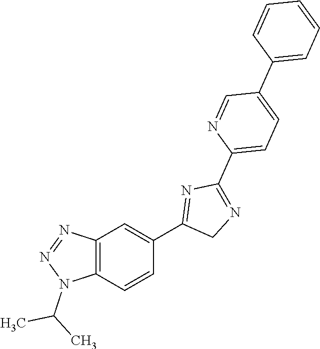 [SMILES: CC(C)n1nnc2cc(C3=NC(c4ccc(-c5ccccc5)cn4)=NC3)ccc21] | 9 | 5-[3-(5-phenylpyridin- 2-yl)-1,2,4-oxadiazol-5- yl]-1-(propan-2-yl)- 1H-1,2,3-benzotriazole | 382.43 | 383.1 |
| 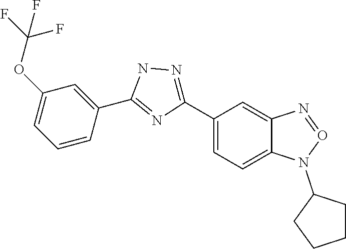 | 10 | 3-(1-cyclopentyl-1H- benzo[d][1,2,3]triazol-5- yl)-5-(3-(trifluoro- methoxy)phenyl)-1,2,4- oxadiazole | 415.38 | 416.1 |
| 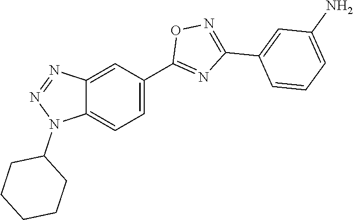 [SMILES: Nc1cccc(-c2noc(-c3ccc4c(c3)nnn4C3CCCCC3)n2)c1] | 11 | 1-cyclohexyl-5-[3-(3- methylphenyl)-1,2,4- oxadiazol-5-yl]-1H-1,2,3- benzotriazole | 359.43 | 360.2 |
| 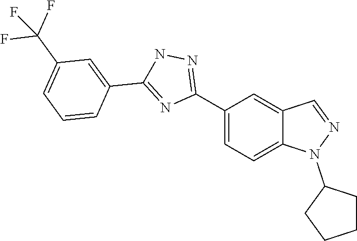 [SMILES: FC(F)(F)c1cccc(-c2nc(-c3ccc4c(cnn4C4CCCC4)c3)n[nH]2)c1] | 12 | 3-(1-cyclopentyl-1H- benzo[d][1,2,3]triazol-5- yl)-5-(3-(trifluoro- methyl)phenyl)-1,2,4- oxadiazole | 399.38 | 400.1 |
| 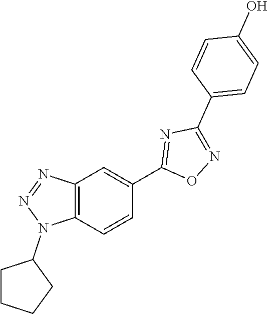 [SMILES: Oc1ccc(-c2noc(-c3ccc4c(c3)nnn4C3CCCC3)n2)cc1] | 13 | 4-[5-(1-cyclopentyl-1H- 1,2,3-benzotriazol-5-yl)- 1,2,4-oxadiazol-3- yl]phenol | 347.38 | 348.1 |
| 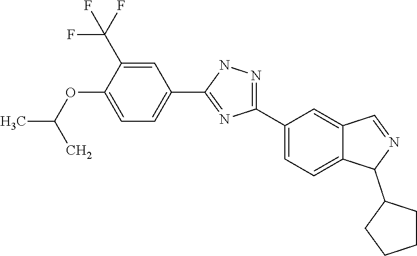 [SMILES: CC(C)Oc1ccc(-c2nc(-c3ccc4c(c3)C=NC4C3CCCC3)n[nH]2)cc1C(F)(F)F] | 14 | 3-(1-cyclopentyl-1H- benzo[d][1,2,3]triazol-5- yl)-5-(4-isopropoxy-3- (trifluoromethyl)phenyl)- 1,2,4-oxadiazole | 457.46 | 458.1 |
| 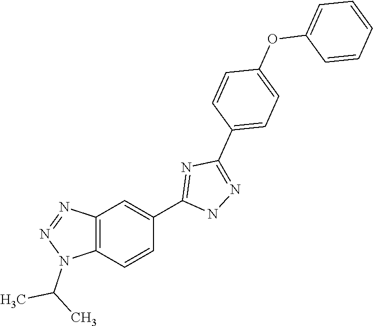 [SMILES: CC(C)n1nnc2cc(-c3nc(-c4ccc(Oc5ccccc5)cc4)n[nH]3)ccc21] | 15 | 5-[3-(4-phenoxyphenyl)- 1,2,4-oxadiazol-5-yl]- 1-(propan-2-yl)- 1H-1,2,3-benzotriazole | 397.44 | 398.2 |
| 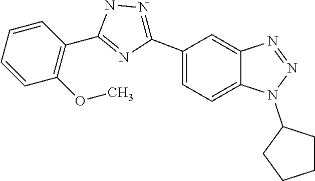 [SMILES: COc1ccccc1-c1nc(-c2ccc3c(c2)nnn3C2CCCC2)n[nH]1] | 16 | 3-(1-cyclopentyl-1H- benzo[d][1,2,3]triazol-5- yl)-5-(2-(methylthio) phenyl)-1,2,4- oxadiazole | 377.47 | 378.1 |
| 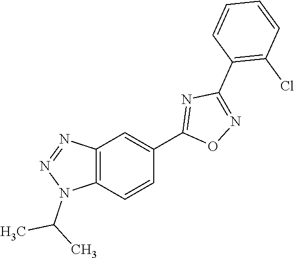 [SMILES: CC(C)n1nnc2cc(-c3nc(-c4ccccc4Cl)no3)ccc21] | 17 | 5-[3-(2-chlorophenyl)- 1,2,4-oxadiazol-5-yl]- 1- propan-2-yl)-1H- 1,2,3-benzotriazole | 339.78 | 340.1 |
| 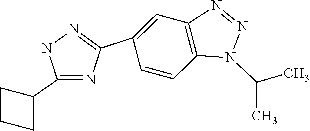 [SMILES: CC(C)n1nnc2cc(-c3n[nH]c(C4CCC4)n3)ccc21] | 18 | 5-(5-cyclobutyl-1,2,4- oxadiazol-3-yl)-1- (propan-2-yl)-1H-1,2,3- benzotriazole | 283.34 | 284 |
| 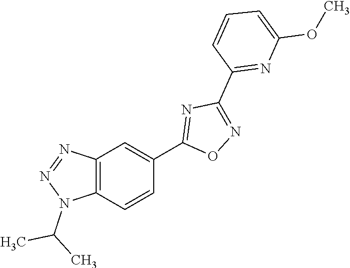 [SMILES: COc1cccc(-c2noc(-c3ccc4c(c3)nnn4C(C)C)n2)n1] | 19 | 5-[3-(6-methoxypyridin- 2-yl)-1,2,4-oxadiazol-5- yl]-1-(propan-2-yl)- 1H-1,2,3-benzotriazole | 336.36 | 337.1 |
| 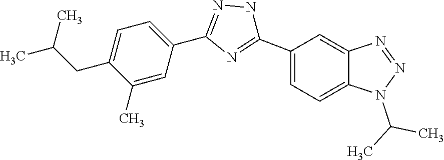 [SMILES: Cc1cc(-c2n[nH]c(-c3ccc4c(c3)nnn4C(C)C)n2)ccc1CC(C)C] | 20 | 3-(4-isopropoxy-3- (trifluoromethyl)phenyl)- 5-(1-isopropyl-1H- benzo[d][1,2,3]triazol-5- yl)-1,2,4-oxadiazole | 431.42 | 432 |
| 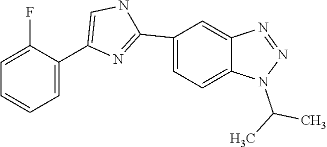 [SMILES: CC(C)n1nnc2cc(-c3nc(-c4ccccc4F)c[nH]3)ccc21] | 21 | 5-[4-(2-fluorophenyl)- 1,3-oxazol-2-yl]-1- (propan-2-yl)-1H-1,2,3- benzotriazole | 322.34 | 323 |
| 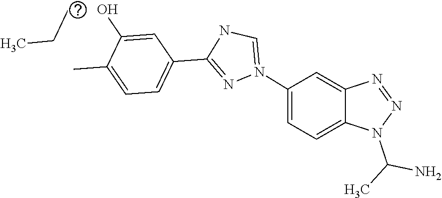 | 22 | 2-isopropoxy-5-(3-(1- isopropyl-1H-benzo[d] [1,2,3]triazol-5-yl)- 1,2,4-oxadiazol-5- yl)benzonitrile | 388.43 | 389 |
| 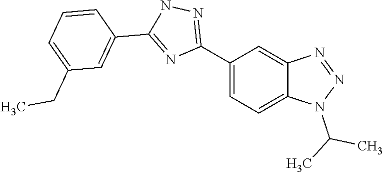 [SMILES: CCc1cccc(-c2nc(-c3ccc4c(c3)nnn4C(C)C)n[nH]2)c1] | 23 | 3-(1-isopropyl-1H- benzo[d][1,2,3]triazol- 5-yl)-5-(3-(phenoxy- methyl)phenyl)- 1,2,4-oxadiazole | 411.47 | 412 |
| 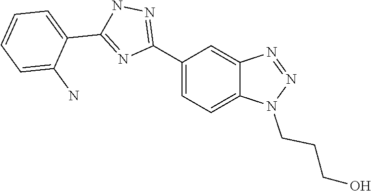 [SMILES: Nc1ccccc1-c1nc(-c2ccc3c(c2)nnn3CCCO)n[nH]1] | 24 | 3-(5-(5-(2-bromophenyl)- 1,2,4-oxadiazol-3-yl)-1H- benzo[d][1,2,3]triazol- 1-yl)propan-1-ol | 400.24 | 400 |
| 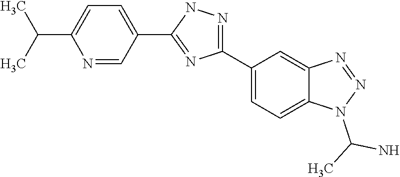 [SMILES: CC(C)c1ccc(-c2nc(-c3ccc4c(c3)nnn4C(C)N)n[nH]2)cn1] | 25 | 3-(1-isopropyl-1H- benzo[d][1,2,3]triazol- 5-yl)-5-(6-isopropyl- pyridin-3-yl)-1,2,4- oxadiazole | 348.41 | 349.3 |
| 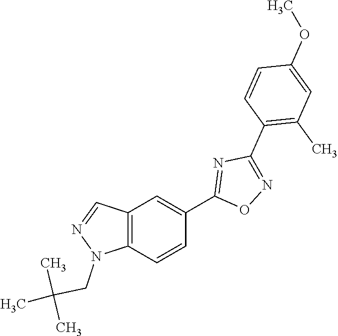 [SMILES: COc1ccc(-c2noc(-c3ccc4c(cnn4CC(C)(C)C)c3)n2)c(C)c1] | 26 | 1-{5-[3-(4-methoxy- 2-methylphenyl)- 1,2,4-oxadiazol- 5-yl]-1H-1,2,3-benzo- triazol-1-yl}-2- methylpropan-2-ol | 379.42 | 380.1 |
| 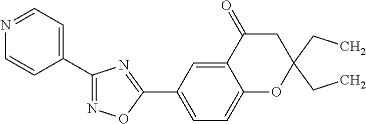 [SMILES: CCC1(CC)CC(=O)c2cc(-c3nc(-c4ccncc4)no3)ccc2O1] | 27 | 2,2-diethyl-6-[3-(pyridin- 4-yl)-1,2,4-oxadiazol- 5-yl]-3,4-dihydro- 2H-1-benzopyran- 4-one | 349.39 | 350.1 |
| 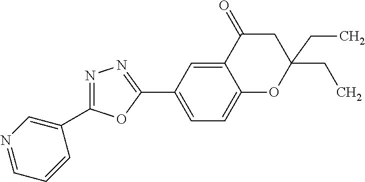 [SMILES: CCC1(CC)CC(=O)c2cc(-c3nnc(-c4cccnc4)o3)ccc2O1] | 28 | 2,2-diethyl-6-(5-(pyridin- 3-yl)-1,3,4-oxadiazol- 2-yl)chroman-4-one | 349.39 | 350.1 |
| 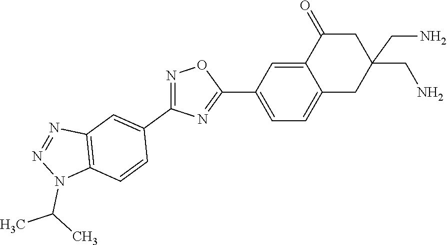 [SMILES: CC(C)n1nnc2cc(-c3noc(-c4ccc5c(c4)C(=O)CC(CN)(CN)C5)n3)ccc21] | 29 | 2,2-diethyl-6-(3-(1- isopropyl-1H- benzo[d][1,2,3]triazol- 5-yl)-1,2,4-thiadiazol- 5-yl)chroman-4-one | 447.56 | 448.2 |
| 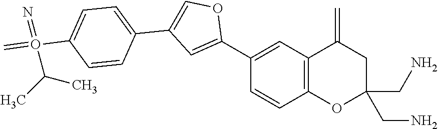 | 30 | 4-(5-(2,2-diethyl-4- oxochroman-6-yl)-1,2,4- oxadiazol-3-yl)-N,N- dimethylbenzene- sulfonamide | 455.53 | 456.1 |
| 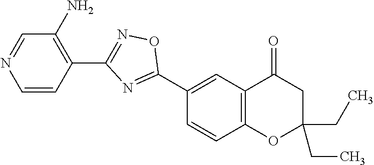 [SMILES: CCC1(CC)CC(=O)c2cc(-c3nc(-c4ccncc4N)no3)ccc2O1] | 31 | 2,2-diethyl-6-(3-(3- methylpyridin-4- yl)-1,2,4-oxadiazol-5- yl)chroman-4-one | 363.42 | 364.2 |
| 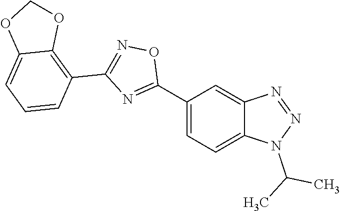 [SMILES: CC(C)n1nnc2cc(-c3nc(-c4cccc5c4OCO5)no3)ccc21] | 32 | 3-(benzo[d][1,3]dioxol- 4-yl)-5-(1-isopropyl- 1H-benzo[d][1,2,3] triazol-5-yl)-1,2,4- oxadiazole | 349.35 | 350.1 |
| 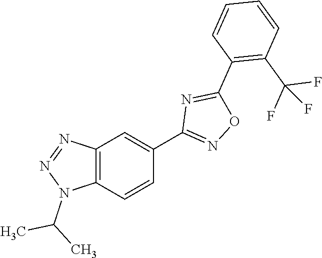 [SMILES: CC(C)n1nnc2cc(-c3noc(-c4ccccc4C(F)(F)F)n3)ccc21] | 33 | 3-(1-isopropyl- benzotriazol-5-yl)-5-[2- (trifluoromethyl)phenyl]- 1,2,4-oxadiazole | 373.34 | 374.1 |
| 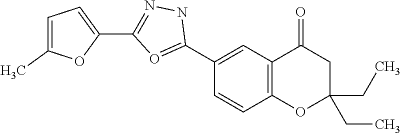 | 34 | 2,2-diethyl-6-(3-(5- methylthiophen-2-yl)- 1,2,4-oxadiazol-5- yl)chroman-4-one | 368.45 | 369.3 |
| 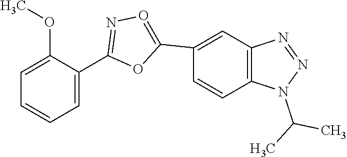 | 35 | 5-[5-(2-methoxyphenyl)- 1,3,4-oxadiazol-2-yl]- 1-(propan-2-yl)-1H- 1,2,3-benzotriazole | 335.37 | 336 |
| 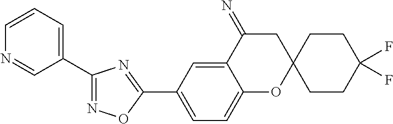 [SMILES: N=C1CC2(CCC(F)(F)CC2)Oc2ccc(-c3nc(-c4cccnc4)no3)cc21] | 36 | 4′,4′-difluoro-6-[3- (pyridin-3-yl)-1,2,4- oxadiazol-5-yl]-3,4- dihydrospiro[1-benzo- pyran-2,1′-cyclohexane]- 4-one | 397.38 | 298.1 |
| 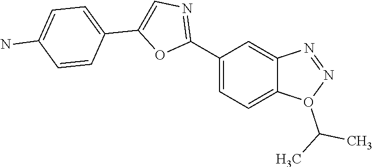 | 37 | 5-(4-fluorophenyl)-2-(1- isopropyl-benzotriazol-5- yl)thiazole | 338.40 | 339.2 |
| 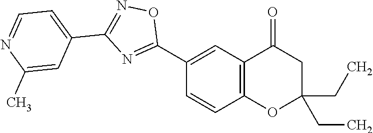 [SMILES: CCC1(CC)CC(=O)c2cc(-c3nc(-c4ccnc(C)c4)no3)ccc2O1] | 38 | 2,2-diethyl-6-(3-(2- methoxypyridin-4-yl)- 1,2,4-oxadiazol-5- yl)chroman-4-one | 379.42 | 380.1 |
| 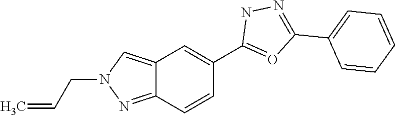 | 39 | 5-(3-phenyl-1,2,4- oxadiazol-5-yl)-2- (prop-2-en-1-yl)-2H- 1,2,3-benzotriazole | 303.33 | 304.3 |
| 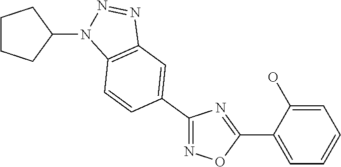 [SMILES: Oc1ccccc1-c1nc(-c2ccc3c(c2)nnn3C2CCCC2)no1] | 40 | 5-(2-bromophenyl)-3- (1-cyclopentyl-1H- benzo[d][1,2,3]triazol- 5-yl)-1,2,4-oxadiazole | 410.28 | 409.9 |
| 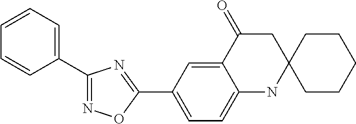 [SMILES: O=C1CC2(CCCCC2)Nc2ccc(-c3nc(-c4ccccc4)no3)cc21] | 41 | 6-[3-(pyridin-3-yl)- 1,2,4-oxadiazol-5- yl]-3,4-dihydrospiro[1- benzopyran-2,1′- cyclohexane]-4-one | 361.40 | 362.1 |
| 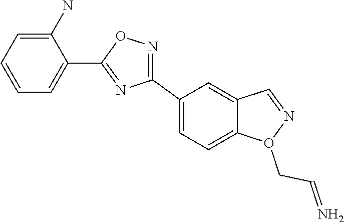 | 42 | 3-(1-allyl-1H- benzo[d][1,2,3]triazol- 5-yl)-5-(2-bromo- phenyl)-1,2,4-oxadiazole | 382.22 | 382 |
| 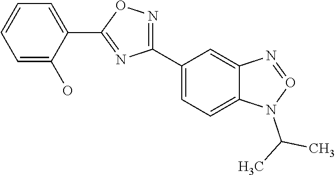 | 43 | 5-(2-bromophenyl)- 3-(1-isopropyl-1H- benzo[d][1,2,3]triazol- 5-yl)-1,2,4-oxadiazole | 384.24 | 384 |
| 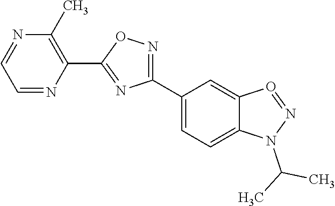 | 44 | 3-(1-isopropyl-1H- benzo[d][1,2,3]triazol-5- yl)-5-(3-methylpyrazin- 2-yl)-1,2,4-oxadiazole | 321.34 | 322.1 |
| 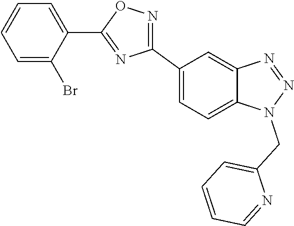 [SMILES: Brc1ccccc1-c1nc(-c2ccc3c(c2)nnn3Cc2ccccn2)no1] | 45 | 5-(2-bromophenyl)- 3-(1-(pyridin- 2-ylmethyl)-1H- benzo[d][1,2,3]triazol- 5-yl)-1,2,4-oxadiazole | 433.27 | 435.1 |
| 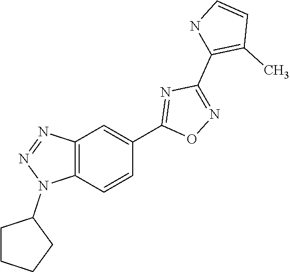 [SMILES: Cc1cc[nH]c1-c1noc(-c2ccc3c(c2)nnn3C2CCCC2)n1] | 46 | 1-cyclopentyl-5-[3-(3- methylthiophen-2-yl)- 1,2,4-oxadiazol-5-yl]- 1H-1,2,3-benzotriazole | 351.43 | 352.1 |
| 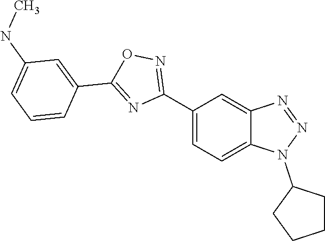 [SMILES: CNc1cccc(-c2nc(-c3ccc4c(c3)nnn4C3CCCC3)no2)c1] | 47 | 3-(1-cyclopentyl-1H- benzo[d][1,2,3]triazol-5- yl)-5-(3-(methylthio) phenyl)-1,2,4- oxadiazole | 377.47 | 378.1 |
| 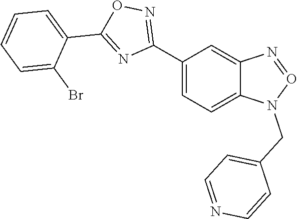 | 48 | 5-(2-bromophenyl)-3- (1-(pyridin-4- ylmethyl)-1H- benzo[d][1,2,3]triazol- 5-yl)-1,2,4-oxadiazole | 433.27 | 435 |
| 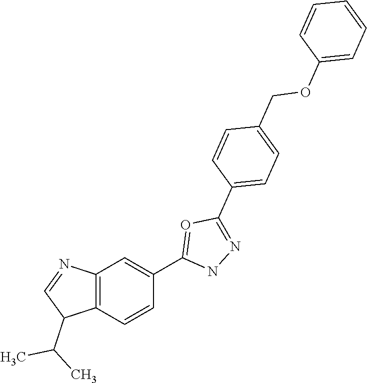 | 49 | 5-{3-[4-(phenoxy- methyl)phenyl]- 1,2,4-oxadiazol-5-yl}- 1-(propan-2-yl)-1H- 1,2,3-benzotriazole | 411.47 | 412.3 |
| 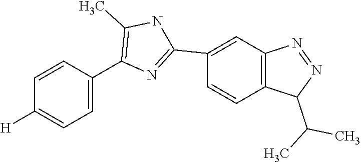 [SMILES: Cc1[nH]c(-c2ccc3c(c2)N=NC3C(C)C)nc1-c1ccccc1] | 50 | 5-[4-(4-chlorophenyl)- 5-methyl-1,3-oxazol- 2-yl]-1-(propan-2-yl)- 1H-1,2,3-benzotriazole | 352.82 | 353 |
| 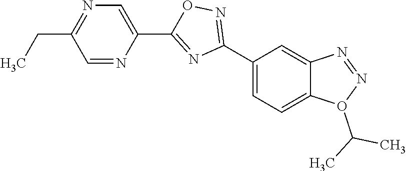 | 51 | 3-(1-isopropyl-1H- benzo[d][1,2,3]triazol-5- yl)-5-(5-methoxypyrazin- 2-yl)-1,2,4-oxadiazole | 337.34 | 338 |
| 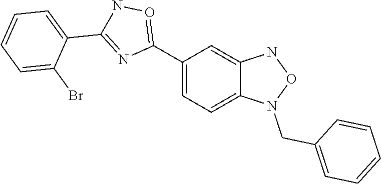 | 52 | 3-(1-benzyl-1H- benzo[d][1,2,3]triazol-5- yl)-5-(2-bromophenyl)- 1,2,4-oxadiazole | 432.28 | 432 |
| 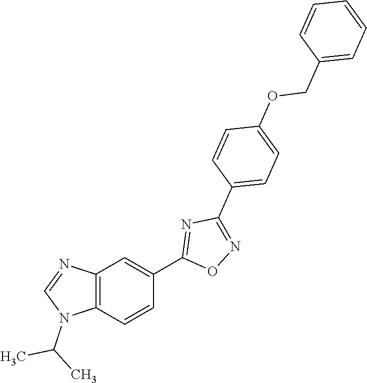 [SMILES: CC(C)n1cnc2cc(-c3nc(-c4ccc(OCc5ccccc5)cc4)no3)ccc21] | 53 | 5-{3-[4-(benzyloxy) phenyl]-1,2,4-oxadiazol- 5-yl}-1-(propan-2-yl)- 1H-1,2,3-benzotriazole | 411.47 | 412.3 |
| 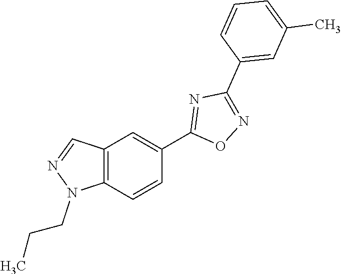 [SMILES: CCCn1ncc2cc(-c3nc(-c4cccc(C)c4)no3)ccc21] | 54 | 5-[3-(3-methylphenyl)- 1,2,4-oxadiazol-5-yl]- 1-propyl-1H-1,2,3- benzotriazole | 319.37 | 320.2 |
| 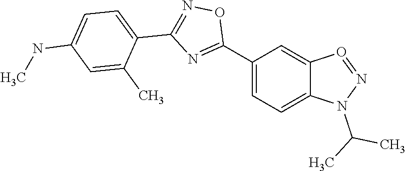 | 55 | 5-(1-isopropyl-1H- benzo[d][1,2,3]triazol-5- yl)-3-(4-methoxy-2- methylphenyl)-1,2,4- oxadiazole | 349.39 | 350 |
| 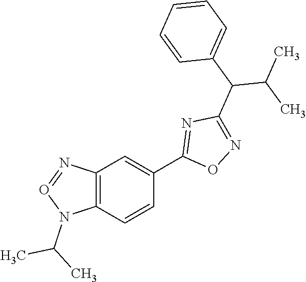 | 56 | 5-[3-(2-methyl-1-phenyl- propyl)-1,2,4-oxadiazol- 5-yl]-1-(propan-2-yl)- 1H-1,2,3-benzotriazole | 361.45 | 362.1 |
| 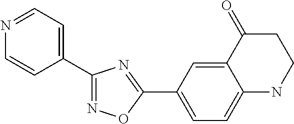 [SMILES: O=C1CCNc2ccc(-c3nc(-c4ccncc4)no3)cc21] | 57 | 6-[3-(pyridin-4-yl)-1,2,4- oxadiazol-5-yl]-3,4- dihydro-2H-1-benzopyran- 4-one | 293.28 | 294.1 |
| 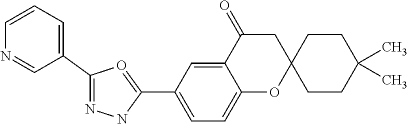 | 58 | 4′,4′-dimethyl-6-[3- (pyridin-3-yl)-1,2,4- oxadiazol-5-yl]-3,4- dihydrospiro[1- benzopyran-2,1′- cyclohexane]-4-one | 389.46 | 390.2 |
| 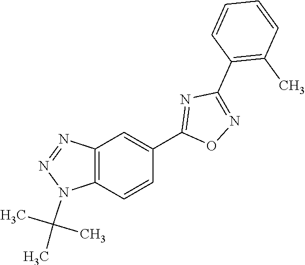 [SMILES: Cc1ccccc1-c1noc(-c2ccc3c(c2)nnn3C(C)(C)C)n1] | 59 | 1-tert-butyl-5-[3-(2- methylphenyl)-1,2,4- oxadiazol-5-yl]-1H- 1,2,3-benzotriazole | 333.40 | 334.1 |
| 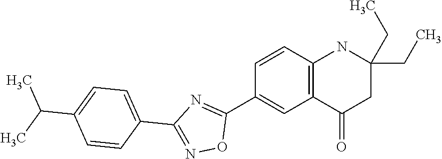 [SMILES: CCC1(CC)CC(=O)c2cc(-c3nc(-c4ccc(C(C)C)cc4)no3)ccc2N1] | 60 | 6-(3-(4-(dimethyl- amino)phenyl)-1,2,4- oxadiazol-5-yl)-2,2- diethylchroman-4-one | 391.47 | 392.3 |
| 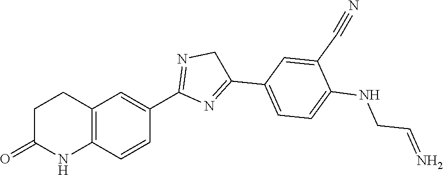 [SMILES: N#Cc1cc(C2=NC(c3ccc4c(c3)CCC(=O)N4)=NC2)ccc1NCC=N] | 61 | 2-(allylamino)-5-(3-(2- oxo-1,2,3,4- tetrahydroquinolin-6-yl)- 1,2,4-oxadiazol-5- yl)benzonitrile | 371.40 | 372.1 |
| 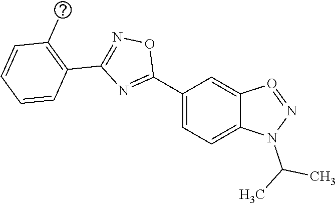 | 62 | 3-(2-isopropoxyphenyl)- 5-(1-isopropyl-1H- benzo[d][1,2,3]triazol- 5-yl)-1,2,4-oxadiazole | 363.42 | 364.1 |
| 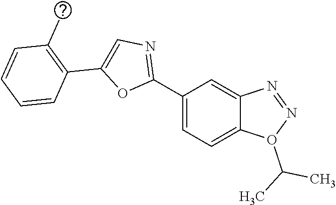 | 63 | 5-(2-fluorophenyl)-2- (1-isopropyl-1H- benzo[d][1,2,3]triazol- 5-yl)thiazole | 338.40 | 339.1 |
| 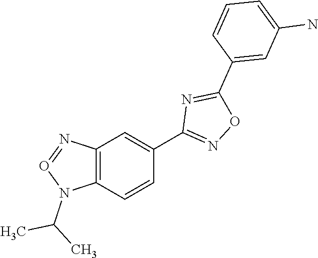 | 64 | 5-(3-fluorophenyl)-3- (1-isopropyl- benzotriazol-5-yl)- 1,2,4-oxadiazole | 323.33 | 324.1 |
| 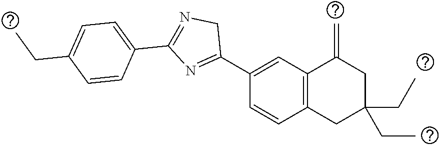 [SMILES: C=C1CC(CC)(CC)Cc2ccc(C3=NC(c4ccc(CC)cc4)=NC3)cc21] | 65 | 2,2-diethyl-6-(3-(4- methoxyphenyl)-1,2,4- oxadiazol-5- yl)chroman-4-one | 378.43 | 379.1 |
| 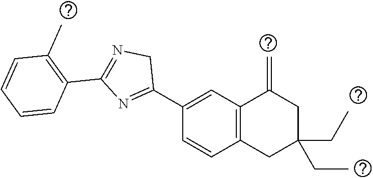 [SMILES: C=C1CC(CC)(CC)Cc2ccc(C3=NC(c4ccccc4C)=NC3)cc21] | 66 | 2,2-diethyl-6-(3-(o- tolyl)-1,2,4-oxadiazol- 5-yl)chroman-4-one | 362.43 | 363.1 |
| 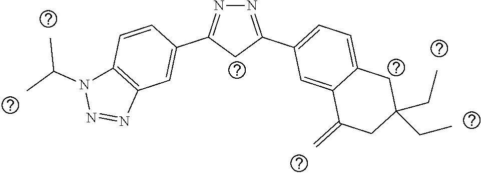 [SMILES: C=C1CC(CC)(CC)Cc2ccc(C3=NN=C(c4ccc5c(c4)nnn5C(C)C)C3)cc21] | 67 | 2,2-diethyl-6-(5-(1- isopropyl-1H- benzo[d][1,2,3]triazol- 5-yl)-1,3,4-thiadiazol- 2-yl)chroman-4-one | 447.56 | 448.1 |
| 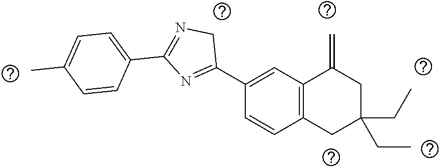 [SMILES: C=C1CC(CC)(CC)Cc2ccc(C3=NC(c4ccc(C)cc4)=NC3)cc21] | 68 | 6-[3-(4-chlorophenyl)- 1,2,4-oxadiazol-5- yl]-2,2-diethyl- chroman-4-one | 382.84 | 383.1 |
| 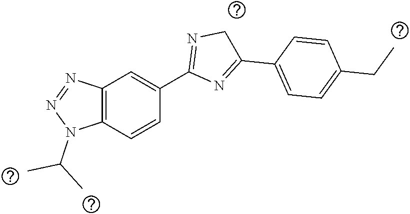 [SMILES: CCc1ccc(C2=NC(c3ccc4c(c3)nnn4C(C)C)=NC2)cc1] | 69 | 3-(1-isopropyl- benzotriazol-5-yl)- 5-(4-methoxyphenyl)- 1,2,4-oxadiazole | 335.37 | 336.1 |
| 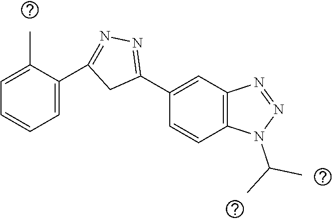 [SMILES: Cc1ccccc1C1=NN=C(c2ccc3c(c2)nnn3C(C)C)C1] | 70 | 2-{5-[1-(propan-2- yl)-1H-1,2,3- benzotriazol-5-yl]- 1,3,4-thiadiazol- 2-yl}phenol | 337.40 | 338 |
| 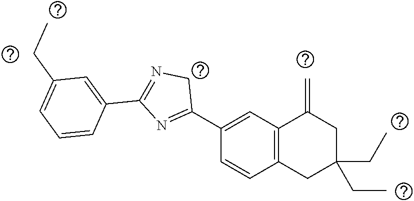 [SMILES: C=C1CC(CC)(CC)Cc2ccc(C3=NC(c4cccc(CC)c4)=NC3)cc21] | 71 | 2,2-diethyl-6-(3-(3- methoxyphenyl)- 1,2,4-oxadiazol- 5-yl)chroman-4-one | 378.43 | 379.1 |
| 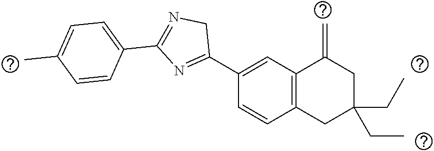 [SMILES: C=C1CC(CC)(CC)Cc2ccc(C3=NC(c4ccc(C)cc4)=NC3)cc21] | 72 | 2,2-diethyl-6-[3-(4- hydroxyphenyl)- 1,2,4-oxadiazol- 5-yl]chroman-4-one | 364.40 | 365.1 |
| 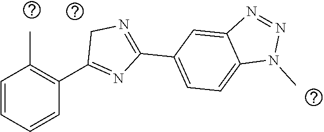 [SMILES: Cc1ccccc1C1=NC(c2ccc3c(c2)nnn3C)=NC1] | 73 | 5-(2-bromophenyl)-3- (1-methyl-1H- benzo[d][1,2,3]triazol-5- yl)-1,2,4-oxadiazole | 356.18 | 356 |
| 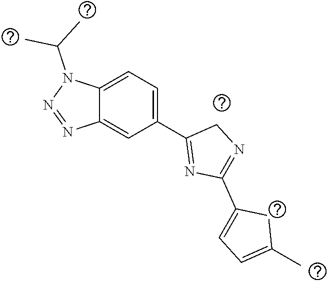 [SMILES: CC1=CC=C(C2=NCC(c3ccc4c(c3)nnn4C(C)C)=N2)C1] | 74 | 5-[5-(5-methylthiophen- 2-yl)-1,2,4-oxadiazol- 3-yl]-1-(propan-2-yl)- 1H-1,2,3-benzotriazole | 325.39 | 326.1 |
| 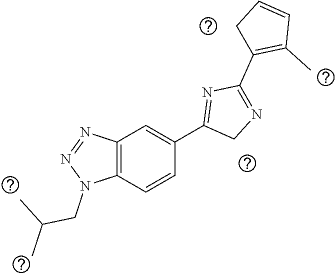 [SMILES: CC1=C(C2=NCC(c3ccc4c(c3)nnn4CC(C)C)=N2)CC=C1] | 75 | 1-(2-methylpropyl)-5-[3- (3-methylthiophen-2-yl)- 1,2,4-oxadiazol-5-yl]- 1H-1,2,3-benzotriazole | 339.42 | 340.1 |
| 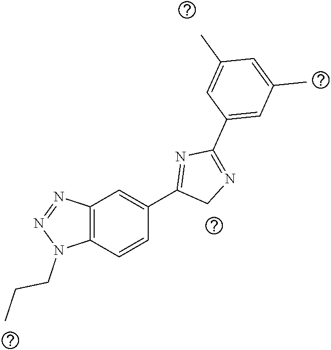 [SMILES: CCCn1nnc2cc(C3=NC(c4cc(C)cc(C)c4)=NC3)ccc21] | 76 | 5-[3-(3,5-dimethyl- phenyl)-1,2,4-oxadiazol- 5-yl]-1-propyl-1H- 1,2,3-benzotriazole | 333.40 | 334.3 |
| 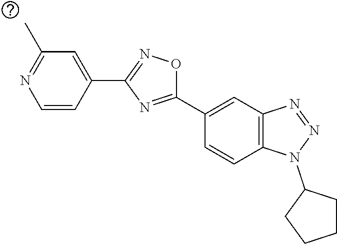 [SMILES: Cc1cc(-c2noc(-c3ccc4c(c3)nnn4C3CCCC3)n2)ccn1] | 77 | 5-[3-(2-methoxypyridin- 4-yl)-1,2,4-oxadiazol- 5-yl]-1-methyl-2,3- dihydro-1H-1,2,3- benzotriazole; cyclopentane | 346.39 | 347.1 |
| 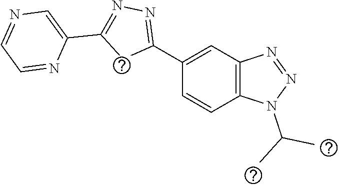 [SMILES: CC(C)n1nnc2cc(C3=NN=C(c4cnccn4)C3)ccc21] | 78 | 3-(1-isopropyl-1H- benzo[d][1,2,3]triazol-5- yl)-5-(pyrazin-2-yl)- 1,2,4-oxadiazole | 307.32 | 308 |
| 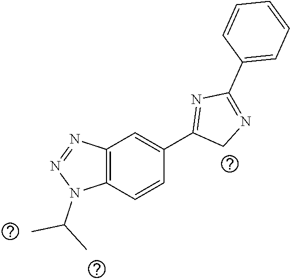 [SMILES: CC(C)n1nnc2cc(C3=NC(c4ccccc4)=NC3)ccc21] | 79 | 5-(3-phenyl-1,2,4- oxadiazol-5-yl)-1- (propan-2-yl)-1H-1,2,3- benzotriazole | 305.34 | 306.3 |
| 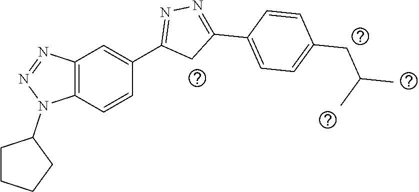 [SMILES: CC(C)Cc1ccc(C2=NN=C(c3ccc4c(c3)nnn4C3CCCC3)C2)cc1] | 80 | 1-cyclopentyl-5-{3- [4-(difluoromethoxy) phenyl]-1,2,4- oxadiazol-5-yl}-1H- 1,2,3-benzotriazole | 397.39 | 398.1 |
| 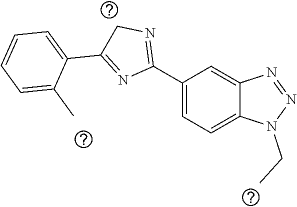 [SMILES: CCn1nnc2cc(C3=NCC(c4ccccc4C)=N3)ccc21] | 81 | methyl 2-(5-(5-(2- bromophenyl)-1,2,4- oxadiazol-3-yl)-1H- benzo[d][1,2,3]triazol- 1-yl)acetate | 414.22 | 414.1 |
| 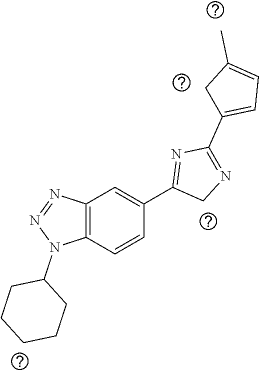 [SMILES: CC1=CC=C(C2=NCC(c3ccc4c(c3)nnn4C3CCCCC3)=N2)C1] | 82 | 5-[3-(5-chlorothiophen- 2-yl)-1,2,4-oxadiazol- 5-yl]-1-(oxan-4-yl)- 1H-1,2,3-benzotriazole | 387.84 | 388 |
| 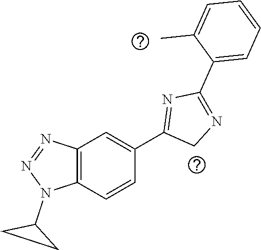 [SMILES: Cc1ccccc1C1=NCC(c2ccc3c(c2)nnn3C2CC2)=N1] | 83 | 5-[3-(2-bromophenyl)- 1,2,4-oxadiazol-5- yl]-1-cyclopropyl- 1H-1,2,3-benzotriazole | 382.22 | 382.2 |
| 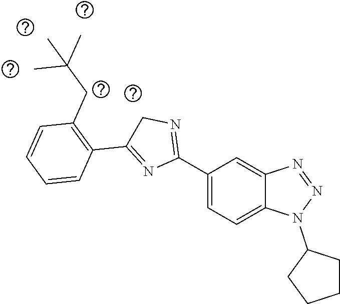 [SMILES: CC(C)(C)Cc1ccccc1C1=NC(c2ccc3c(c2)nnn3C2CCCC2)=NC1] | 84 | 3-(1-cyclopentyl-1H- benzo[d][1,2,3]triazol-5- yl)-5-(2-(trifluoro- methoxy)phenyl)-1,2,4- oxadiazole | 415.38 | 416 |
| 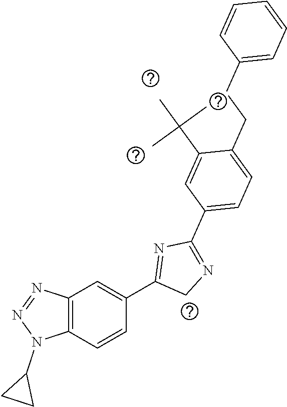 [SMILES: CC(C)(C)c1cc(C2=NCC(c3ccc4c(c3)nnn4C3CC3)=N2)ccc1CCc1ccccc1] | 85 | 5-{3-[4-(benzyloxy)-3- (trifluoromethyl)phenyl]- 1,2,4-oxadiazol-5-yl}- 1-cyclopropyl-1H- 1,2,3-benzotriazole | 477.45 | 478.2 |
| 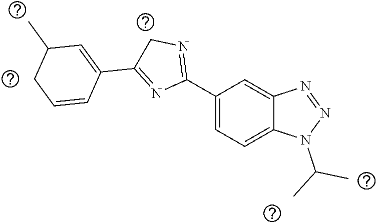 [SMILES: CC1C=C(C2=NC(c3ccc4c(c3)nnn4C(C)C)=NC2)C=CC1] | 86 | 3-(1-isopropyl-1H- benzo[d][1,2,3]triazol-5- yl)-5-(2-methyl- pyridin-4-yl)-1,2,4- oxadiazole | 322.37 | 321.1 |
| 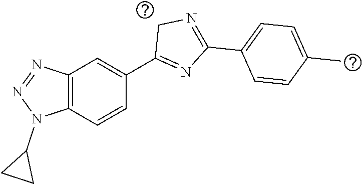 [SMILES: Cc1ccc(C2=NCC(c3ccc4c(c3)nnn4C3CC3)=N2)cc1] | 87 | 1-cyclopropyl-5-[3-(6- methylpyridin-3-yl)-1,2,4- oxadiazol-5-yl]-1H-1,2,3- benzotriazole | 318.34 | 319.1 |
| 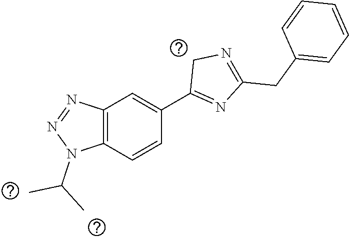 [SMILES: CC(C)n1nnc2cc(C3=NC(Cc4ccccc4)=NC3)ccc21] | 88 | 5-(3-benzyl-1,2,4- oxadiazol-5-yl)-1- (propan-2-yl)-1H-1,2,3- benzotriazole | 319.37 | 320.1 |
| 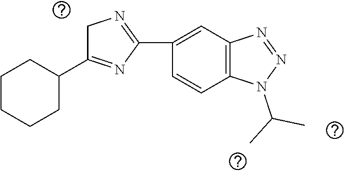 [SMILES: CC(C)n1nnc2cc(C3=NCC(C4CCCCC4)=N3)ccc21] | 89 | 5-[5-(oxan-4-yl)- 1,2,4-oxadiazol-3-yl]- 1-(propan-2-yl)-1H- 1,2,3-benzotriazole | 313.36 | 314 |
| 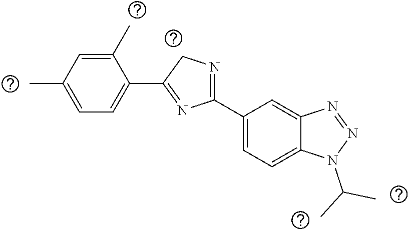 [SMILES: Cc1ccc(C2=NC(c3ccc4c(c3)nnn4C(C)C)=NC2)c(C)c1] | 90 | 3-(1-isopropyl-1H- benzo[d][1,2,3]triazol- 5-yl)-5-(4-methoxy- 2-methylphenyl)-1,2,4- oxadiazole | 349.39 | 350.1 |
| 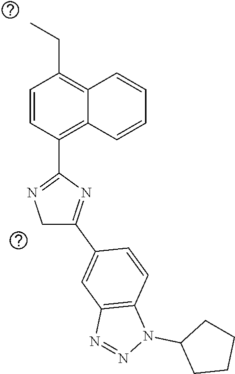 [SMILES: CCc1ccc(C2=NCC(c3ccc4c(c3)nnn4C3CCCC3)=N2)c2ccccc12] | 91 | 1-cyclopentyl-5-[3-(4- methoxynaphthalen-1- yl)-1,2,4- oxadiazol-5-yl]-1H- 1,2,3-benzotriazole | 411.47 | 412.2 |
| 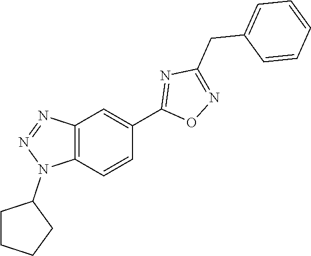 [SMILES: c1ccc(Cc2noc(-c3ccc4c(c3)nnn4C3CCCC3)n2)cc1] | 92 | 5-(3-benzyl-1,2,4- oxadiazol-5-yl)-1- cyclopentyl-1H-1,2,3- benzotriazole | 345.41 | 346.1 |
| 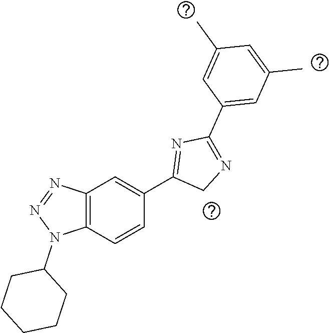 [SMILES: Cc1cc(C)cc(C2=NCC(c3ccc4c(c3)nnn4C3CCCCC3)=N2)c1] | 93 | 5-[3-(3,5-dimethyl- phenyl)-1,2,4- oxadiazol-5-yl]-1- (oxan-4-yl)-1H- 1,2,3-benzotriazole | 375.43 | 376.2 |
| 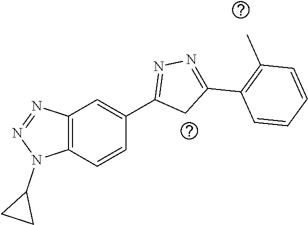 [SMILES: Cc1ccccc1C1=NN=C(c2ccc3c(c2)nnn3C2CC2)C1] | 94 | 1-cyclopropyl-5-[3- (4-methylpyridin-3-yl)- 1,2,4-oxadiazol-5- yl]-1H-1,2,3- benzotriazole | 318.34 | 319.3 |
| 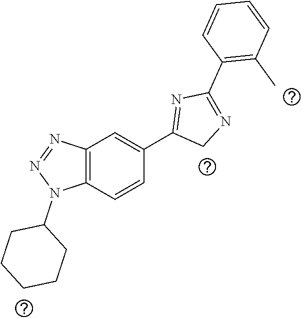 [SMILES: Cc1ccccc1C1=NCC(c2ccc3c(c2)nnn3C2CCCCC2)=N1] | 95 | 5-[3-(2-methylphenyl)- 1,2,4-oxadiazol-5-yl]- 1-(oxan-4-yl)-1H- 1,2,3-benzotriazole | 361.41 | 362.1 |
| 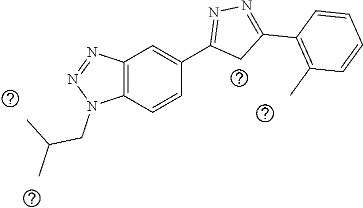 [SMILES: Cc1ccccc1C1=NN=C(c2ccc3c(c2)nnn3CC(C)C)C1] | 96 | 5-[3-(2-methylphenyl)- 1,2,4-oxadiazol-5-yl]- 1-(2-methylpropyl)-1H- 1,2,3-benzotriazole | 333.40 | 334.2 |
| 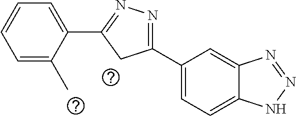 [SMILES: Cc1ccccc1C1=NN=C(c2ccc3[nH]nnc3c2)C1] | 97 | 3-(1H-benzo[d][1,2,3] triazol-5-yl)-5-(2- bromophenyl)-1,2,4- oxadiazole | 342.16 | 343.9 |
| 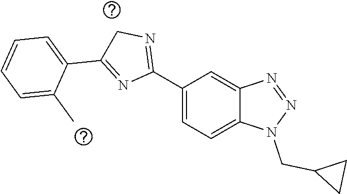 [SMILES: Cc1ccccc1C1=NC(c2ccc3c(c2)nnn3CC2CC2)=NC1] | 98 | 5-(2-bromophenyl)-3-(1- (cyclopropylmethyl)-1H- benzo[d][1,2,3]triazol-5- yl)-1,2,4-oxadiazole | 396.25 | 396 |
| 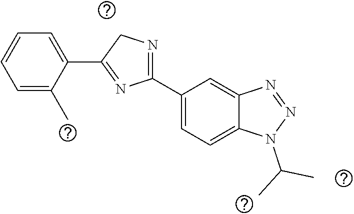 [SMILES: Cc1ccccc1C1=NC(c2ccc3c(c2)nnn3C(C)C)=NC1] | 99 | 3-(1-isopropyl-1H- benzo[d][1,2,3]triazol- 5-yl)-5-(o-tolyl)-1,2,4- oxadiazole | 319.37 | 320 |
| 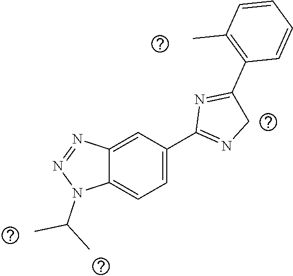 [SMILES: Cc1ccccc1C1=NC(c2ccc3c(c2)nnn3C(C)C)=NC1] | 100 | 3-(1-isopropyl-1H- benzo[d][1,2,3]triazol- 5-yl)-5-(4-methyl- pyridin-3-yl)-1,2,4- oxadiazole | 320.36 | 321.1 |
| 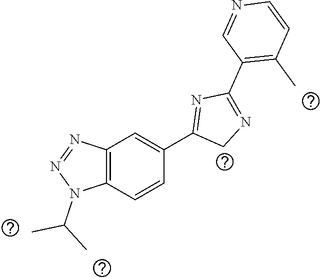 [SMILES: Cc1ccncc1C1=NCC(c2ccc3c(c2)nnn3C(C)C)=N1] | 101 | 5-[3-(4-methylpyridin- 3-yl)-1,2,4-oxadiazol- 5-yl]-1-(propan-2-yl)- 1H-1,2,3-benzotriazole | 320.36 | 321.1 |
| 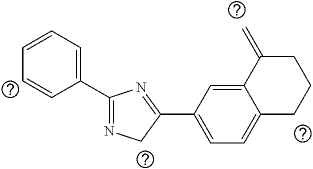 [SMILES: C=C1CCCc2ccc(C3=NC(c4ccccc4)=NC3)cc21] | 102 | 6-[3-(pyridin-3-yl)-1,2,4- oxadiazol-5-yl]-3,4- dihydro-2H-1- benzopyran-4-one | 293.28 | 294.1 |
| 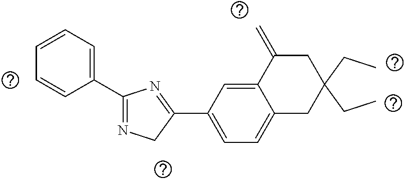 [SMILES: C=C1CC(CC)(CC)Cc2ccc(C3=NC(c4ccccc4)=NC3)cc21] | 103 | 2,2-diethyl-6-[3-(pyridin- 3-yl)-1,2,4-oxadiazol- 5-yl]-3,4-dihydro-2H- 1-benzopyran-4-one | 349.39 | 350.2 |
| 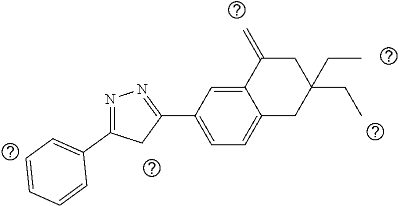 [SMILES: C=C1CC(CC)(CC)Cc2ccc(C3=NN=C(c4ccccc4)C3)cc21] | 104 | 2,2-diethyl-6-(5- (pyridin-3-yl)-1,3,4- thiadiazol-2-yl)chroman- 4-one | 365.45 | 366.1 |
| 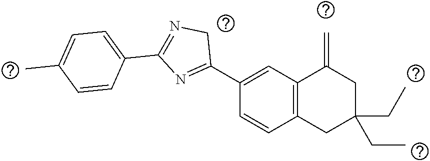 [SMILES: C=C1CC(CC)(CC)Cc2ccc(C3=NC(c4ccc(C)cc4)=NC3)cc21] | 105 | 2,2-diethyl-6-[3-(6- hydroxy-3-pyridyl)- 1,2,4-oxadiazol-5- yl]chroman-4-one | 365.39 | 366.1 |
| 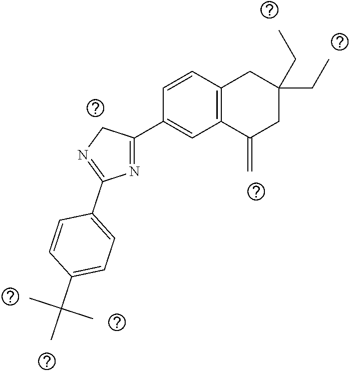 [SMILES: C=C1CC(CC)(CC)Cc2ccc(C3=NC(c4ccc(C(C)(C)C)cc4)=NC3)cc21] | 106 | 4-(5-(2,2-diethyl-4- oxochroman-6-yl)- 1,2,4-oxadiazol-3- yl)benzenesulfonamide | 427.48 | 428.1 |
| 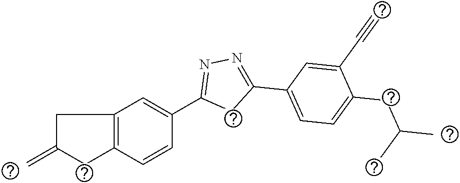 [SMILES: C#Cc1cc(C2=NN=C(c3ccc4c(c3)CC(=C)C4)C2)ccc1CC(C)C] | 107 | 2-(isopropylamino)-5- (3-(2-oxoindolin-5-yl)- 1,2,4-oxadiazol- 5-yl)benzonitrile | 359.39 | 360.1 |
| 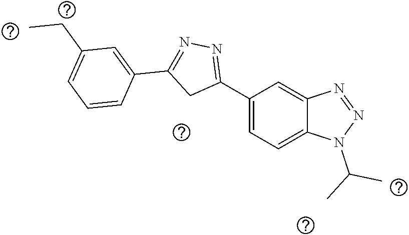 [SMILES: CCc1cccc(C2=NN=C(c3ccc4c(c3)nnn4C(C)C)C2)c1] | 108 | 2-(1-isopropyl-1H- benzo[d][1,2,3]triazol- 5-yl)-5-(3-methoxy- phenyl)thiazole | 350.44 | 351.2 |
| 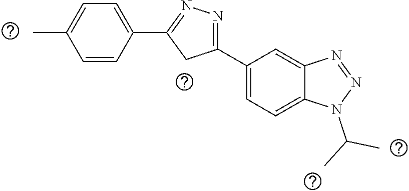 [SMILES: Cc1ccc(C2=NN=C(c3ccc4c(c3)nnn4C(C)C)C2)cc1] | 109 | 2-(1-isopropyl-1H- benzo[d][1,2,3]triazol- 5-yl)-5-(4-methoxy- phenyl)thiazole | 350.44 | 351.1 |
| 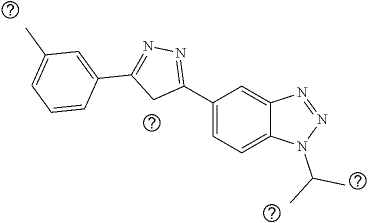 [SMILES: Cc1cccc(C2=NN=C(c3ccc4c(c3)nnn4C(C)C)C2)c1] | 110 | 5-(3-fluorophenyl)-2- (1-isopropyl-1H- benzo[d][1,2,3]triazol- 5-yl)thiazole | 338.40 | 339.1 |
| 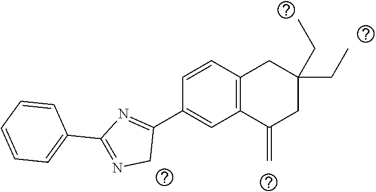 [SMILES: C=C1CC(CC)(CC)Cc2ccc(C3=NC(c4ccccc4)=NC3)cc21] | 111 | 2,2-diethyl-6-[3-(2- pyridyl)-1,2,4- oxadiazol-5-yl]chroman- 4-one | 349.39 | 350.1 |
| 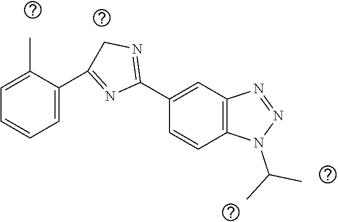 [SMILES: Cc1ccccc1C1=NC(c2ccc3c(c2)nnn3C(C)C)=NC1] | 112 | 2-{3-[1-(propan-2-yl)- 1H-1,2,3-benzotriazol- 5-yl]-1,2,4-thiadiazol- 5-yl}phenol | 337.40 | 338 |
| 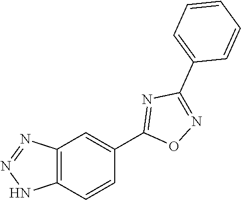 [SMILES: c1ccc(-c2noc(-c3ccc4[nH]nnc4c3)n2)cc1] | 113 | 5-(3-phenyl-1,2,4- oxadiazol-5-yl)-1H- 1,2,3-benzotriazole | 263.26 | 264.1 |
| 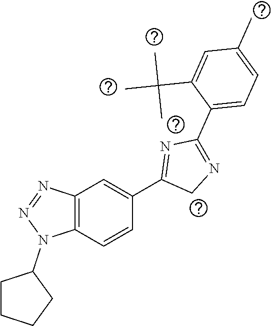 [SMILES: Cc1ccc(C2=NCC(c3ccc4c(c3)nnn4C3CCCC3)=N2)c(C(C)(C)C)c1] | 114 | 5-{3-[4-bromo-2- (trifluoromethyl)phenyl]- 1,2,4-oxadiazol-5- yl}-1-cyclopentyl- 1H-1,2,3-benzotriazole | 478.27 | 478.1 |
| 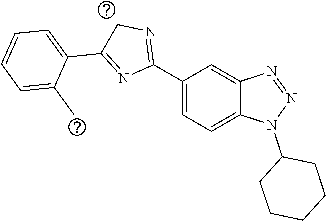 [SMILES: Cc1ccccc1C1=NC(c2ccc3c(c2)nnn3C2CCCCC2)=NC1] | 115 | 5-(2-bromophenyl)-3-(1- cyclohexyl-1H- benzo[d][1,2,3]triazol-5- yl)-1,2,4-oxadiazole | 424.30 | 426 |
| 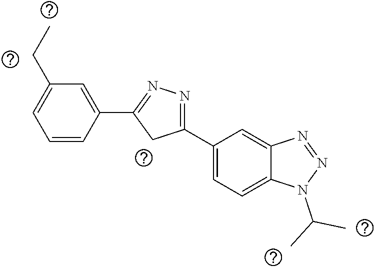 [SMILES: CCc1cccc(C2=NN=C(c3ccc4c(c3)nnn4C(C)C)C2)c1] | 116 | 3-(1-isopropyl-1H- benzo[d][1,2,3]triazol-5- yl)-5-(2-methoxypyridin- 4-yl)-1,2,4-oxadiazole | 336.36 | 337 |
| 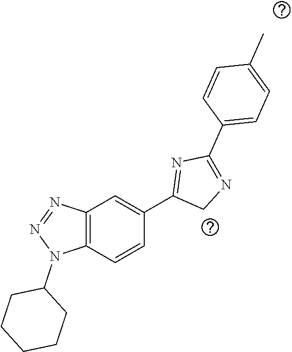 [SMILES: Cc1ccc(C2=NCC(c3ccc4c(c3)nnn4C3CCCCC3)=N2)cc1] | 117 | 1-cyclohexyl-5-[3-(4- methylphenyl)-1,2,4- oxadiazol-5-yl]-1H- 1,2,3-benzotriazole | 359.43 | 360.1 |
| 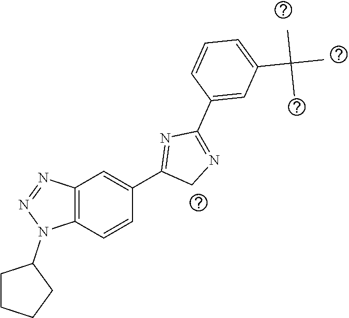 [SMILES: CC(C)(C)c1cccc(C2=NCC(c3ccc4c(c3)nnn4C3CCCC3)=N2)c1] | 118 | 1-cyclopentyl-5-{3- [3-(trifluoromethyl) phenyl]-1,2,4-oxadiazol- 5-yl}-1H-1,2,3- benzotriazole | 399.38 | 400.1 |
| 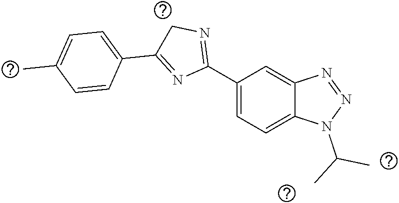 [SMILES: Cc1ccc(C2=NC(c3ccc4c(c3)nnn4C(C)C)=NC2)cc1] | 119 | 3-(1-isopropyl-1H- benzo[d][1,2,3]triazol- 5-yl)-5-(6-methyl- pyridin-3-yl)-1,2,4- oxadiazole | 320.36 | 321.1 |
| 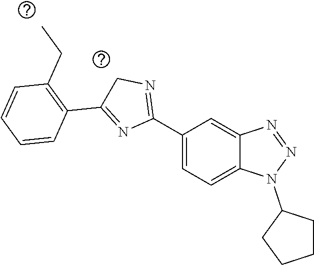 [SMILES: CCc1ccccc1C1=NC(c2ccc3c(c2)nnn3C2CCCC2)=NC1] | 120 | 3-(1-cyclopentyl- 1H-benzo[d][1,2,3] triazol-5-yl)-5-(2- methoxyphenyl)- 1,2,4-oxadiazole | 361.41 | 362 |
| 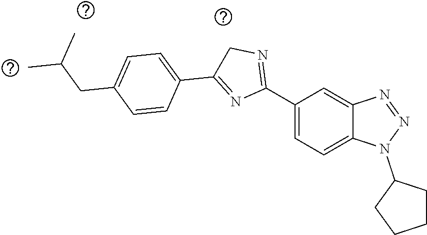 [SMILES: CC(C)Cc1ccc(C2=NC(c3ccc4c(c3)nnn4C3CCCC3)=NC2)cc1] | 121 | 3-(1-cyclopentyl- 1H-benzo[d][1,2,3] triazol-5-yl)-5-(4- isopropoxyphenyl)- 1,2,4-oxadiazole | 389.46 | 390.3 |
| 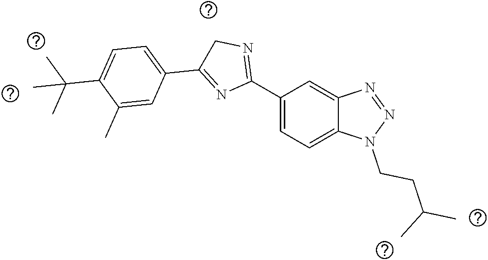 [SMILES: Cc1cc(C2=NC(c3ccc4c(c3)nnn4CCC(C)C)=NC2)ccc1C(C)(C)C] | 122 | 5-{3-[3-fluoro-4- (trifluoromethyl)phenyl]- 1,2,4-oxadiazol-5-yl}- 1-(3-methyl-butyl)- 1H-1,2,3-benzotriazole | 419.38 | 420.2 |
| 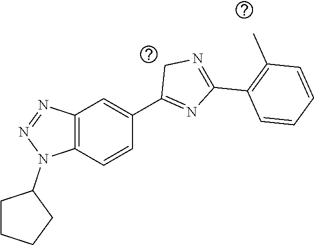 [SMILES: Cc1ccccc1C1=NCC(c2ccc3c(c2)nnn3C2CCCC2)=N1] | 123 | 5-[3-(3,5-dimethyl- phenyl)-1,2,4-oxadiazol- 5-yl]-1-(propan-2-yl)- 1H-1,2,3-benzotriazole | 345.41 | 346.2 |
| 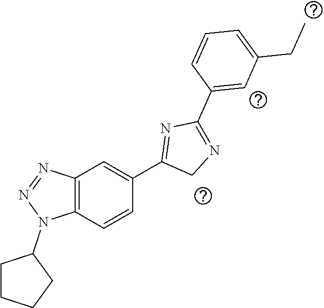 [SMILES: CCc1cccc(C2=NCC(c3ccc4c(c3)nnn4C3CCCC3)=N2)c1] | 124 | 1-cyclopentyl-5-[3-(6- methoxypyridin-2-yl)- 1,2,4-oxadiazol-5-yl]- 1H-1,2,3-benzotriazole | 362.39 | 363.1 |
| 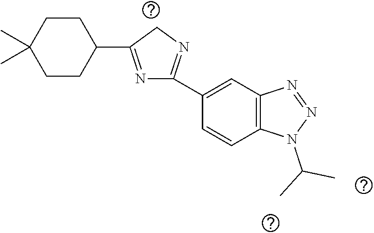 [SMILES: CC(C)n1nnc2cc(C3=NCC(C4CCC(C)(C)CC4)=N3)ccc21] | 125 | 5-[5-(4,4-difluoro- cyclohexyl)-1,2,4- oxadiazol-3-yl]-1- (propan-2-yl)-1H- 1,2,3-benzotriazole | 347.37 | 348 |
| 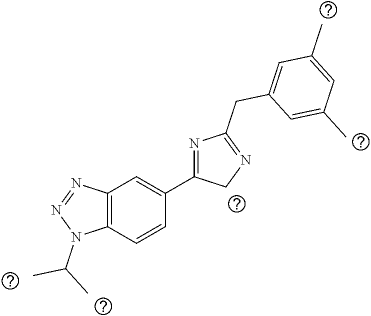 [SMILES: Cc1cc(C)cc(CC2=NCC(c3ccc4c(c3)nnn4C(C)C)=N2)c1] | 126 | 5-{3-[(3,5-dimethyl- phenyl)methyl]- 1,2,4-oxadiazol-5- yl}-1-(propan-2-yl)- 1H-1,2,3-benzotriazole | 347.42 | 348.3 |
| 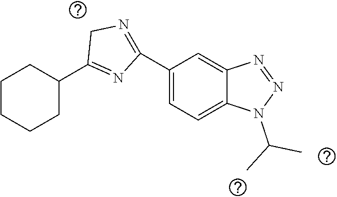 [SMILES: CC(C)n1nnc2cc(C3=NCC(C4CCCCC4)=N3)ccc21] | 127 | 5-(5-cyclohexyl- 1,2,4-oxadiazol-3- yl)-1-(propan-2-yl)-1H- 1,2,3-benzotriazole | 311.39 | 312 |
| 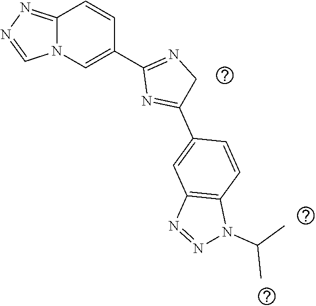 [SMILES: CC(C)n1nnc2cc(C3=NC(c4ccc5nncn5c4)=NC3)ccc21] | 128 | 1-(propan-2-yl)-5-(3- {[1,2,4]triazolo[4,3- a]pyridin-6-yl}-1,2,4- oxadiazol-5-yl)-1H- 1,2,3-benzotriazole | 346.35 | 347.1 |
| 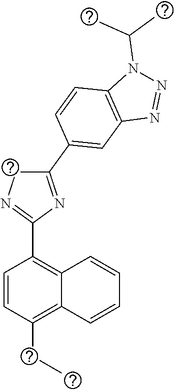 [SMILES: CCc1ccc(C2=NCC(c3ccc4c(c3)nnn4C(C)C)=N2)c2ccccc12] | 129 | 5-[3-(4-methoxy- naphthalen-1-yl)- 1,2,4-oxadiazol-5-yl]- 1-(propan-2-yl)- 1H-1,2,3-benzotriazole | 385.43 | 386.2 |
| 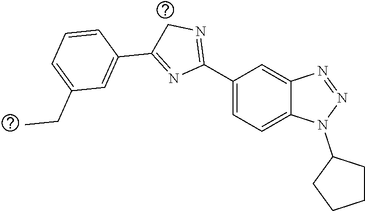 [SMILES: CCc1cccc(C2=NC(c3ccc4c(c3)nnn4C3CCCC3)=NC2)c1] | 130 | 3-(1-cyclopentyl-1H- benzo[d][1,2,3]triazol- 5-yl)-5-(3-methoxy- phenyl)-1,2,4-oxadiazole | 361.41 | 362 |
| 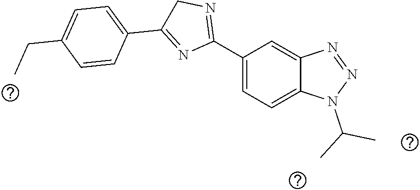 [SMILES: CCc1ccc(C2=NC(c3ccc4c(c3)nnn4C(C)C)=NC2)cc1] | 131 | 3-(1-isopropyl-1H- benzo[d][1,2,3]triazol- 5-yl)-5-(4-(phenoxy- methyl)phenyl)-1,2,4- oxadiazole | 411.47 | 412 |
| 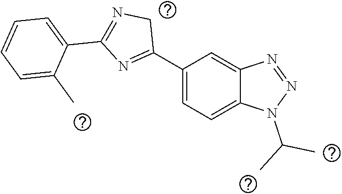 [SMILES: Cc1ccccc1C1=NCC(c2ccc3c(c2)nnn3C(C)C)=N1] | 132 | 5-(1-isopropyl-1H- benzo[d][1,2,3]triazol-5- yl)-3-(2-(trifluoro- methoxy)phenyl)-1,2,4- oxadiazole | 389.34 | 390 |
| 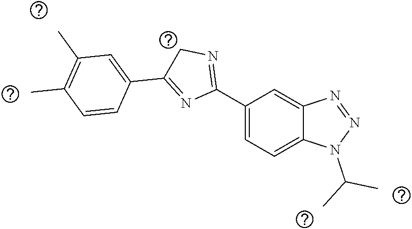 [SMILES: Cc1ccc(C2=NC(c3ccc4c(c3)nnn4C(C)C)=NC2)cc1C] | 133 | 5-(5,6-dimethylpyrazin- 2-yl)-3-(1-isopropyl- 1H-benzo[d][1,2,3] triazol-5-yl)-1,2,4- oxadiazole | 335.37 | 356.1 |
| 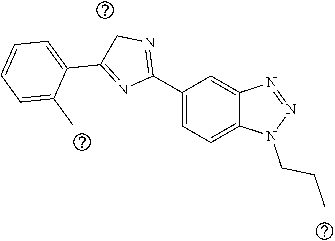 [SMILES: CCCn1nnc2cc(C3=NCC(c4ccccc4C)=N3)ccc21] | 134 | 3-(5-(5-(2-bromophenyl)- 1,2,4-oxadiazol-3-yl)-1H- benzo[d][1,2,3]triazol-1- yl)propanoic acid | 414.22 | 413.9 |
| 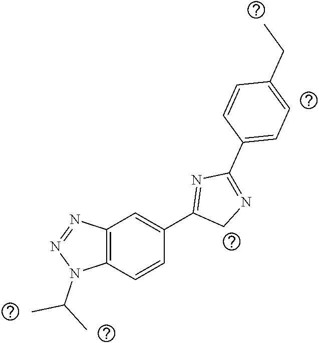 [SMILES: CCc1ccc(C2=NCC(c3ccc4c(c3)nnn4C(C)C)=N2)cc1] | 135 | 5-[3-(2-ethylpyrimidin- 5-yl)-1,2,4-oxadiazol-5- yl]-1-(propan-2-yl)- 1H-1,2,3-benzotriazole | 335.37 | 336.1 |
| 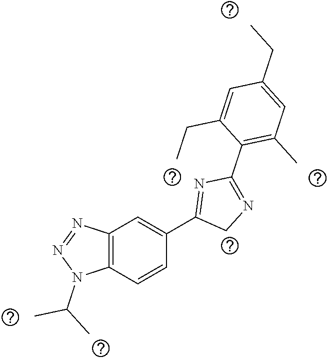 [SMILES: CCc1cc(C)c(C2=NCC(c3ccc4c(c3)nnn4C(C)C)=N2)c(CC)c1] | 136 | 5-[3-(2,4-dimethoxy-6- methylphenyl)-1,2,4- oxadiazol-5-yl]-1- (propan-2-yl)-1H-1,2,3- benzotriazole | 379.42 | 380.3 |
| 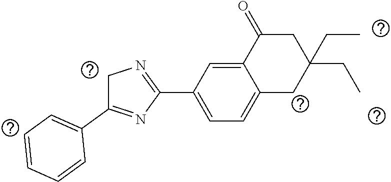 [SMILES: CCC1(CC)CC(=O)c2cc(C3=NCC(c4ccccc4)=N3)ccc2C1] | 137 | 2,2-diethyl-6-(5-(pyridin- 3-yl)-1,2,4-thiadiazol- 3-yl)chroman-4-one | 365.45 | 366 |
| 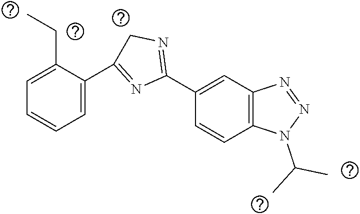 [SMILES: CCc1ccccc1C1=NC(c2ccc3c(c2)nnn3C(C)C)=NC1] | 138 | 5-[5-(2-methoxyphenyl)- 1,2,4-thiadiazol-3-yl]- 1-(propan-2-yl)-1H- 1,2,3-benzotriazole | 351.43 | 302 |
| 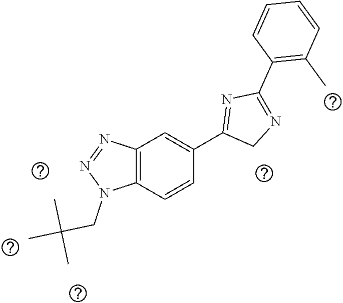 [SMILES: Cc1ccccc1C1=NCC(c2ccc3c(c2)nnn3CC(C)(C)C)=N1] | 139 | 2-methyl-1-{5-[3-(2- methylphenyl)-1,2,4- oxadiazol-5-yl]-1H- 1,2,3-benzotriazol-1- yl}propan-2-ol | 349.39 | 350.1 |
| 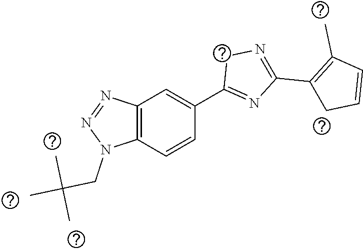 [SMILES: CC1=C(C2=NCC(c3ccc4c(c3)nnn4CC(C)(C)C)=N2)CC=C1] | 140 | 2-methyl-1-{5-[3-(3- methylthiophen-2-yl)- 1,2,4-oxadiazol-5-yl]- 1H-1,2,3-benzotriazol- 1-yl propan-2-ol | 355.42 | 356.1 |
| 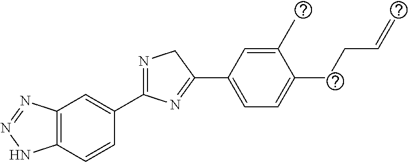 [SMILES: C=CCCc1ccc(C2=NC(c3ccc4[nH]nnc4c3)=NC2)cc1C] | 141 | 5-(3-(1H-indazol-5-yl)- 1,2,4-oxadiazol-5- yl)-2-(allyl- amino)benzonitrile | 342.36 | 343.1 |
| 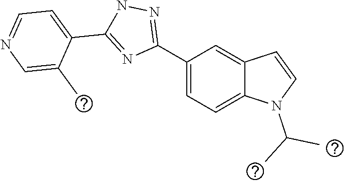 [SMILES: Cc1cnccc1-c1nc(-c2ccc3c(ccn3C(C)C)c2)n[nH]1] | 142 | 3-(1-isopropyl-1H- indol-5-yl)-5-(3- methylpyridin-4-yl)- 1,2,4-oxadiazole | 318.38 | 318.15 |
| 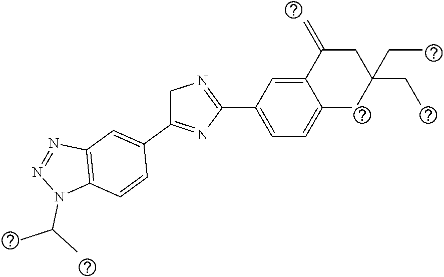 [SMILES: C=C1CC(CC)(CC)Cc2ccc(C3=NCC(c4ccc5c(c4)nnn5C(C)C)=N3)cc21] | 143 | 2,2-diethyl-6-(5-(1- isopropyl-1H- benzo[d][1,2,3]triazol- 5-yl)-1,2,4-thiadiazol- 3-yl)chroman-4-one | 447.56 | 448.1 |
| 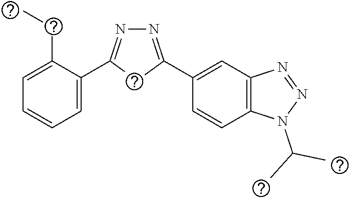 [SMILES: CCc1ccccc1C1=NN=C(c2ccc3c(c2)nnn3C(C)C)C1] | 144 | 5-[5-(2-methoxyphenyl)- 1,3,4-thiadiazol-2- yl]-1-(propan-2-yl)- 1H-1,2,3-benzotriazole | 351.43 | 352 |
| 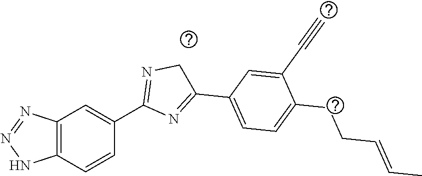 [SMILES: C#Cc1cc(C2=NC(c3ccc4[nH]nnc4c3)=NC2)ccc1CC/C=C/C] | 145 | 5-(3-(1H-benzo[d] [1,2,3]triazol-5-yl)- 1,2,4-oxadiazol-5-yl)-2- (allylamino)benzonitrile | 343.35 | 344.1 |
| 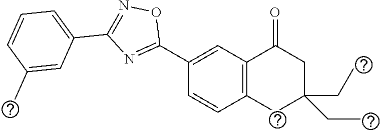 [SMILES: CCC1(CC)CC(=O)c2cc(-c3nc(-c4cccc(C)c4)no3)ccc2C1] | 146 | 2,2-diethyl-6-(3- (2-fluoropyridin-4- yl)-1,2,4-oxadiazol- 5-yl)chroman-4-one | 367.38 | 368.1 |
| 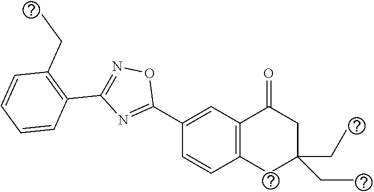 [SMILES: CCc1ccccc1-c1noc(-c2ccc3c(c2)C(=O)CC(CC)(CC)C3)n1] | 147 | 2,2-diethyl-6-(3-(2- methoxypyridin-3-yl)- 1,2,4-oxadiazol-5- yl)chroman-4-one | 379.42 | 380.2 |
| 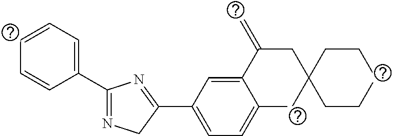 [SMILES: C=C1CC2(CCCCC2)Cc2ccc(C3=NC(c4ccccc4)=NC3)cc21] | 148 | 6-[3-(pyridin-4-yl)- 1,2,4-oxadiazol-5- yl]-3,4-dihydrospiro [1-benzopyran-2,4′- oxane]-4-one | 363.37 | 364.1 |
| 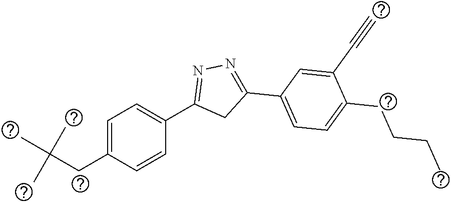 [SMILES: C#Cc1cc(C2=NN=C(c3ccc(CC(C)(C)C)cc3)C2)ccc1CCCC] | 149 | N-(4-(5-(4-(allylamino)- 3-cyanophenyl)-1,2,4- oxadiazol-3-yl)phenyl) methanesulfonamide | 395.44 | 396.1 |
| 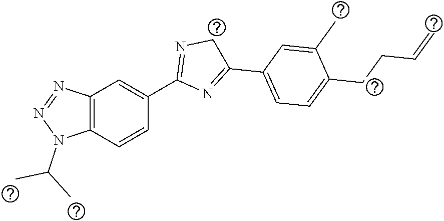 [SMILES: C=CCCc1ccc(C2=NC(c3ccc4c(c3)nnn4C(C)C)=NC2)cc1C] | 150 | 2-(allylamino)-5-(3- (1-isopropyl-1H- benzo[d][1,2,3]triazol- 5-yl)-1,2,4-oxadiazol- 5-yl)benzonitrile | 385.43 | 386.3 |
| 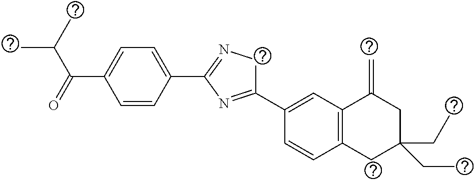 [SMILES: C=C1CC(CC)(CC)Cc2ccc(C3=NC(c4ccc(C(=O)C(C)C)cc4)=NC3)cc21] | 151 | 4-(5-(2,2-diethyl-4- oxochroman-6-yl)- 1,2,4-oxadiazol-3-yl)- N,N-dimethylbenzamide | 419.48 | 420.2 |
| 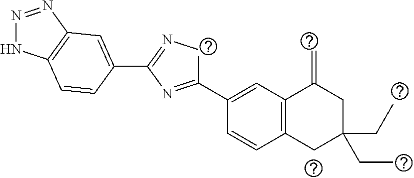 [SMILES: C=C1CC(CC)(CC)Cc2ccc(C3=NC(c4ccc5[nH]nnc5c4)=NC3)cc21] | 152 | 6-(3-(1H-indazol-5-yl)- 1,2,4-oxadiazol- 5-yl)-2,2-diethyl- chroman-4-one | 388.43 | 389.2 |
| 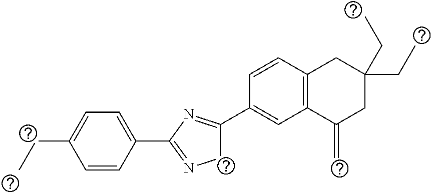 [SMILES: C=C1CC(CC)(CC)Cc2ccc(C3=NC(c4ccc(CC)cc4)=NC3)cc21] | 153 | 2,2-diethyl-6-(3-(4- (methylamino)phenyl)- 1,2,4-oxadiazol-5- yl)chroman-4-one | 377.44 | 378.2 |
| 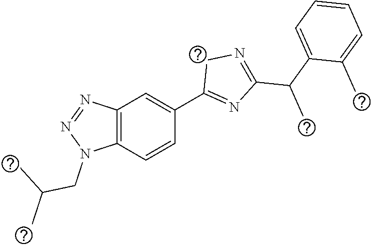 [SMILES: Cc1ccccc1C(C)C1=NCC(c2ccc3c(c2)nnn3CC(C)C)=N1] | 154 | 5-{3-[1-(2-methyl- phenyl)ethyl]-1,2,4- oxadiazol-5-yl}-1-(2- methylpropyl)-1H- 1,2,3-benzotriazole | 361.45 | 362.2 |
| 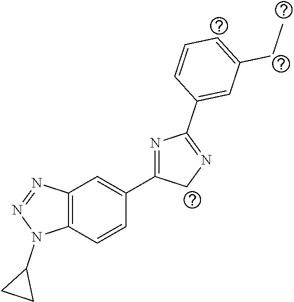 [SMILES: CCc1cccc(C2=NCC(c3ccc4c(c3)nnn4C3CC3)=N2)c1] | 155 | 1-cyclopropyl-5-[3- (2-methoxypyridin- 4-yl)-1,2,4-oxadiazol- 5-yl]-1H-1,2,3- benzotriazole | 334.34 | 335.1 |
| 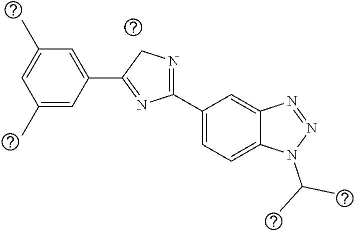 [SMILES: Cc1cc(C)cc(C2=NC(c3ccc4c(c3)nnn4C(C)C)=NC2)c1] | 156 | 5-(2,6-dimethyl- pyridin-4-yl)-3-(1- isopropyl-1H- benzo[d][1,2,3]triazol- 5-yl)-1,2,4- oxadiazole | 334.38 | 335.2 |
| 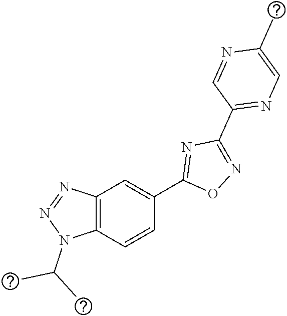 [SMILES: Cc1cnc(-c2noc(-c3ccc4c(c3)nnn4C(C)C)n2)cn1] | 157 | 5-[3-(5-methylpyrazin- 2-yl)-1,2,4-oxadiazol- 5-yl]-1-(propan-2-yl)- 1H-1,2,3-benzotriazole | 321.34 | 322.1 |
| 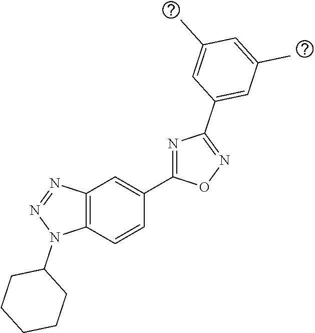 [SMILES: Cc1cc(C)cc(-c2noc(-c3ccc4c(c3)nnn4C3CCCCC3)n2)c1] | 158 | 1-cyclohexyl-5-[3- (3,5-dimethylphenyl)- 1,2,4-oxadiazol-5-yl]- 1H-1,2,3-benzotriazole | 373.46 | 374.2 |
| 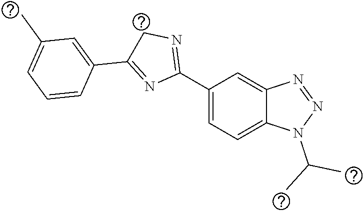 [SMILES: Cc1cccc(C2=NC(c3ccc4c(c3)nnn4C(C)C)=NC2)c1] | 159 | 3-(1-isopropyl-1H- benzo[d][1,2,3]triazol- 5-yl)-5-(5-methylpyridin- 3-yl)-1,2,4-oxadiazole | 320.36 | 321.1 |
| 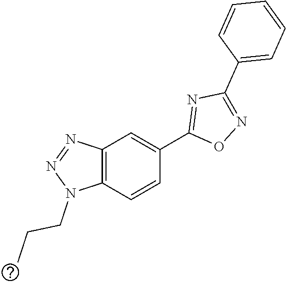 [SMILES: CCCn1nnc2cc(-c3nc(-c4ccccc4)no3)ccc21] | 160 | 5-(3-phenyl-1,2,4- oxadiazol-5-yl)- 1-propyl-1H-1,2,3- benzotriazole | 305.34 | 306.1 |
| 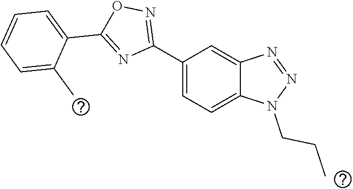 [SMILES: CCCn1nnc2cc(-c3noc(-c4ccccc4C)n3)ccc21] | 161 | methyl 3-(5-(5-(2- bromophenyl)-1,2,4- oxadiazol-3-yl)-1H- benzo[d][1,2,3]triazol- 1-yl)propanoate | 428.25 | 429.9 |
| 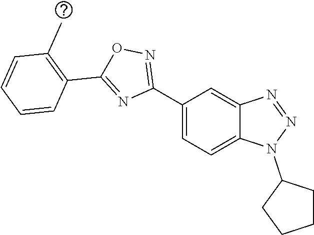 [SMILES: Cc1ccccc1-c1nc(-c2ccc3c(c2)nnn3C2CCCC2)no1] | 162 | 3-(1-cyclopentyl-1H- benzo[d][1,2,3]triazol- 5-yl)-5-(o-tolyl)- 1,2,4-oxadiazole | 345.41 | 346.1 |
| 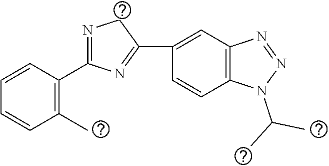 [SMILES: Cc1ccccc1C1=NCC(c2ccc3c(c2)nnn3C(C)C)=N1] | 163 | 5-[4-(2-chlorophenyl)- 2,3-dihydro-1,3- oxazol-2-yl]-1- (propan-2-yl)-1H- 1,2,3-benzotriazole | 338.80 | 339 |
| 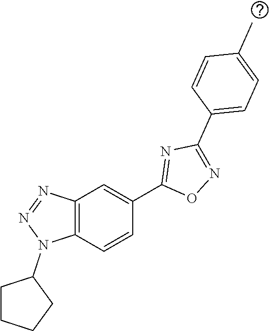 [SMILES: Cc1ccc(-c2noc(-c3ccc4c(c3)nnn4C3CCCC3)n2)cc1] | 164 | 1-cyclopentyl-5-[3- (4-methylphenyl)- 1,2,4-oxadiazol-5-yl]- 1H-1,2,3-benzotriazole | 345.41 | 346.3 |
| 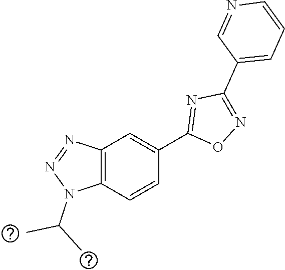 [SMILES: CC(C)n1nnc2cc(-c3nc(-c4cccnc4)no3)ccc21] | 165 | 1-(propan-2-yl)-5-[3- (pyridin-3-yl)-1,2,4- oxadiazol-5-yl]-1H- 1,2,3-benzotriazole | 306.33 | 307.3 |
| 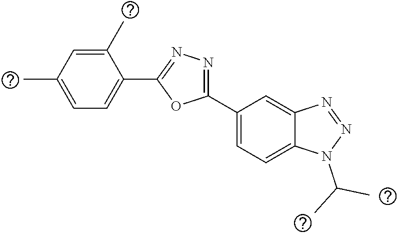 [SMILES: Cc1ccc(-c2nnc(-c3ccc4c(c3)nnn4C(C)C)o2)c(C)c1] | 166 | 5-(2,4-dimethylphenyl)- 3-(1-isopropyl-1H- benzo[d][1,2,3]triazol- 5-yl)-1,2,4-oxadiazole | 333.40 | 334.2 |
| 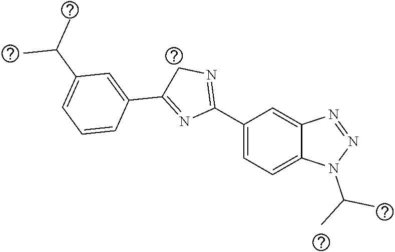 [SMILES: CC(C)c1cccc(C2=NC(c3ccc4c(c3)nnn4C(C)C)=NC2)c1] | 167 | 3-(1-isopropyl-1H- benzo[d][1,2,3]triazol- 5-yl)-5-(2-isopropyl- pyridin-4-yl)-1,2,4- oxadiazole | 348.41 | 349.1 |
| 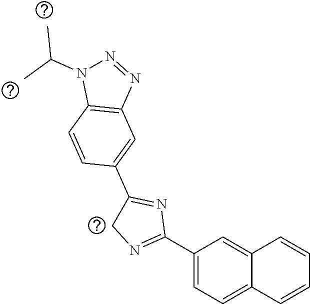 [SMILES: CC(C)n1nnc2cc(C3=NC(c4ccc5ccccc5c4)=NC3)ccc21] | 168 | 2-{5-[1-(propan-2- yl)-1H-1,2,3- benzotriazol-5-yl]- 1,2,4-oxadiazol-3- yl}quinoline | 356.39 | 357.1 |
| 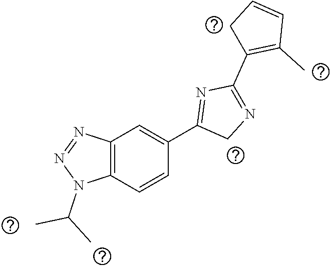 [SMILES: CC1=C(C2=NCC(c3ccc4c(c3)nnn4C(C)C)=N2)CC=C1] | 169 | 5-[3-(3-methyl- thiophen-2-yl)-1,2,4- oxadiazol-5-yl]-1- (propan-2-yl)-1H- 1,2,3-benzotriazole | 325.39 | 326.1 |
| 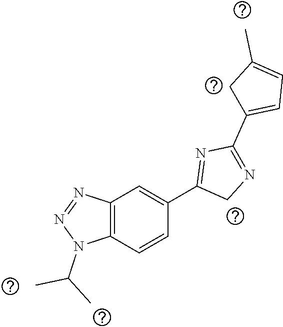 [SMILES: CC1=CC=C(C2=NCC(c3ccc4c(c3)nnn4C(C)C)=N2)C1] | 170 | 5-[3-(5-chlorothiophen- 2-yl)-1,2,4-oxadiazol- 5-yl]-1-(propan-2-yl)- 1H-1,2,3-benzotriazole | 345.81 | 346.1 |
| 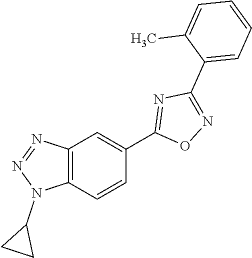 [SMILES: Cc1ccccc1-c1noc(-c2ccc3c(c2)nnn3C2CC2)n1] | 171 | 1-cyclopropyl-5-[3- (2-methylphenyl)- 1,2,4-oxadiazol-5-yl]- 1H-1,2,3-benzotriazole | 317.35 | 318.3 |
| 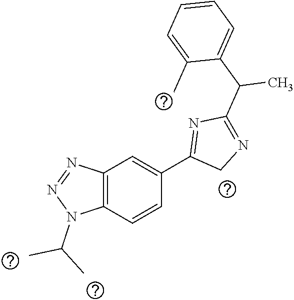 [SMILES: Cc1ccccc1C(C)C1=NCC(c2ccc3c(c2)nnn3C(C)C)=N1] | 172 | 5-{3-[1-(2-methyl- phenyl)ethyl]- 1,2,4-oxadiazol-5-yl}- 1-(propan-2-yl)-1H- 1,2,3-benzotriazole | 347.42 | 348.1 |
| 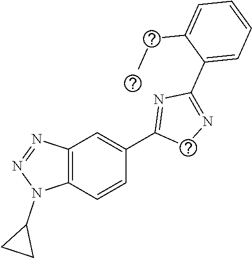 [SMILES: CCc1ccccc1C1=NCC(c2ccc3c(c2)nnn3C2CC2)=N1] | 173 | 1-cyclopropyl-5-[3- (2-methoxyphenyl)- 1,2,4-oxadiazol- 5-yl]-1H-1,2,3- benzotriazole | 333.35 | 334.2 |
| 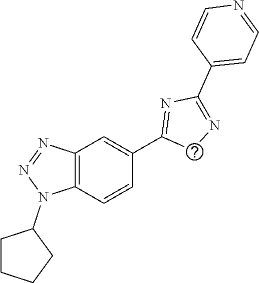 [SMILES: c1cc(C2=NCC(c3ccc4c(c3)nnn4C3CCCC3)=N2)ccn1] | 174 | 1-cyclopentyl-5-[3- (pyridin-4-yl)-1,2,4- oxadiazol-5-yl]-1H- 1,2,3-benzotriazole | 332.37 | 333.1 |
| 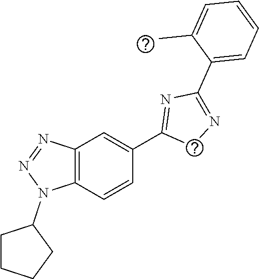 [SMILES: Cc1ccccc1C1=NCC(c2ccc3c(c2)nnn3C2CCCC2)=N1] | 175 | 1-cyclopentyl-5- [3-(2-methylphenyl)- 1,2,4-oxadiazol-5- yl]-1H-1,2,3- benzotriazole | 345.41 | 346.2 |
| 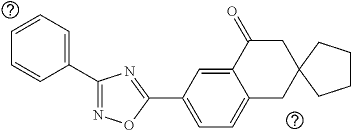 [SMILES: O=C1CC2(CCCC2)Cc2ccc(-c3nc(-c4ccccc4)no3)cc21] | 176 | 6-[3-(pyridin-4-yl)- 1,2,4-oxadiazol-5- yl]-3,4-dihydrospiro[1- benzopyran-2,1′- cyclopentane]-4-one | 347.37 | 348.1 |
| 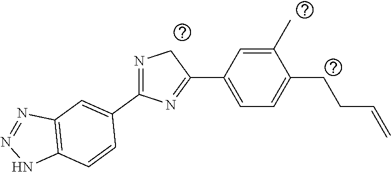 [SMILES: C=CCCc1ccc(C2=NC(c3ccc4[nH]nnc4c3)=NC2)cc1C] | 177 | 5-(3-(1H- benzo[d]imidazol-5-yl)- 1,2,4-oxadiazol-5-yl)-2- (allylamino)benzonitrile | 342.36 | 343.1 |
| 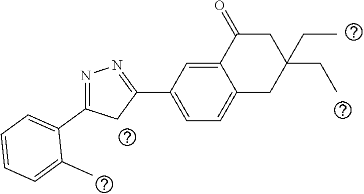 [SMILES: CCC1(CC)CC(=O)c2cc(C3=NN=C(c4ccccc4C)C3)ccc2C1] | 178 | 2,2-diethyl-6-(5-(2- methoxyphenyl)- 1,3,4-thiadiazol-2- yl)chroman-4-one | 394.49 | 395.2 |
| 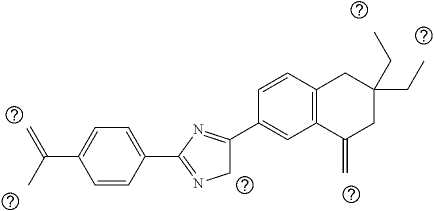 [SMILES: C=C(C)c1ccc(C2=NCC(c3ccc4c(c3)C(=C)CC(CC)(CC)C4)=N2)cc1] | 179 | 4-(5-(2,2-diethyl-4- oxochroman-6- yl)-1,2,4-oxadiazol- 3-yl)benzamide | 391.43 | 392.1 |
| 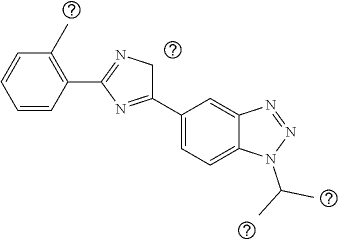 [SMILES: Cc1ccccc1C1=NCC(c2ccc3c(c2)nnn3C(C)C)=N1] | 180 | 2-(5-(1-isopropyl-1H- benzo[d][1,2,3]triazol- 5-yl)-1,2,4-oxadiazol- 3-yl)phenol | 321.34 | 322.1 |
| 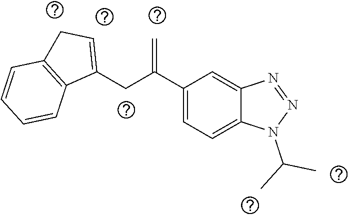 [SMILES: C=C(CC1=CCc2ccccc21)c1ccc2c(c1)nnn2C(C)C] | 181 | N-(benzo[d]isoxazol-3- yl)-1-isopropyl-1H- benzo[d][1,2,3]triazole- 5-carboxamide | 321.34 | 322.1 |
| 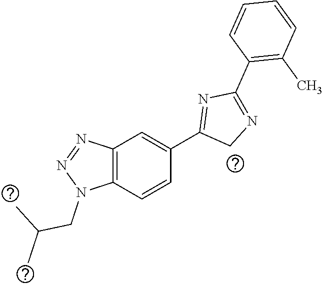 [SMILES: Cc1ccccc1C1=NCC(c2ccc3c(c2)nnn3CC(C)C)=N1] | 182 | 1-(2,2-difluoroethyl)-5- [3-(2-methylphenyl)- 1,2,4-oxadiazol-5- yl]-1H-1,2,3- benzotriazole | 341.32 | 342.1 |
| 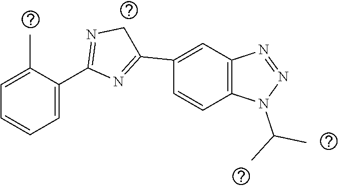 [SMILES: Cc1ccccc1C1=NCC(c2ccc3c(c2)nnn3C(C)C)=N1] | 183 | 5-[4-(2-bromophenyl)- 1,3-oxazol-2-yl]-1- (propan-2-yl)-1H-1,2,3- benzotriazole | 383.25 | 383 |
| 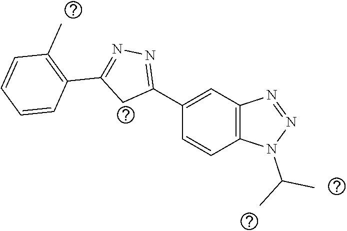 [SMILES: Cc1ccccc1C1=NN=C(c2ccc3c(c2)nnn3C(C)C)C1] | 184 | 2-(1-isopropyl-1H- benzo[d][1,2,3]triazol- 5-yl)-5-(2-methoxy- phenyl)thiazole | 350.44 | 351 |
| 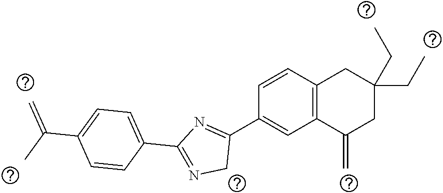 [SMILES: C=C(C)c1ccc(C2=NCC(c3ccc4c(c3)C(=C)CC(CC)(CC)C4)=N2)cc1] | 185 | 4-(5-(2,2-diethyl-4- oxochroman-6-yl)- 1,2,4-oxadiazol-3-yl)- N-methylbenzamide | 405.45 | 406.1 |
| 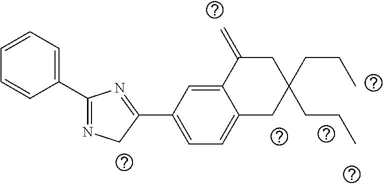 [SMILES: C=C1CC(CCC)(CCC)Cc2ccc(C3=NC(c4ccccc4)=NC3)cc21] | 186 | 2,2-bis(methoxy- methyl)-6-[3-(pyridin- 3-yl)-1,2,4-oxadiazol- 5-yl]-3,4-dihydro-2H- 1-benzopyran-4-one | 381.39 | 282.2 |
| 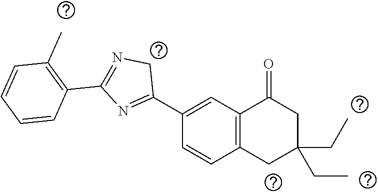 [SMILES: CCC1(CC)CC(=O)c2cc(C3=NC(c4ccccc4C)=NC3)ccc2C1] | 187 | 2,2-diethyl-6-[3- (2-hydroxyphenyl)- 1,2,4-oxadiazol-5- yl]chroman-4-one | 364.40 | 365.1 |
| 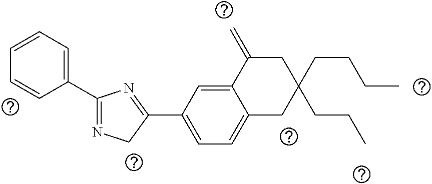 [SMILES: C=C1CC(CCC)(CCCC)Cc2ccc(C3=NC(c4ccccc4)=NC3)cc21] | 188 | 2,2-dibutyl-6-[3- (pyridin-3-yl)-1,2,4- oxadiazol-5-yl]-3,4- dihydro-2H-1-benzo- pyran-4-one | 405.50 | 406.2 |
| 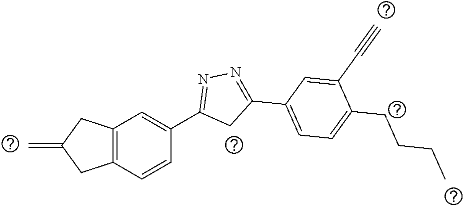 [SMILES: C#Cc1cc(C2=NN=C(c3ccc4c(c3)CC(=C)C4)C2)ccc1CCCC] | 189 | 2-(allylamino)-5-(3- (2-oxo-2,3-dihydro- benzo[d]oxazol-6-yl)- 1,2,4-oxadiazol-5- yl)benzonitrile | 359.35 | 360.1 |
| 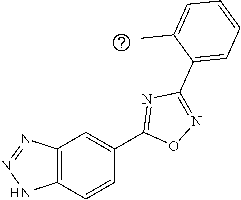 [SMILES: Cc1ccccc1-c1noc(-c2ccc3[nH]nnc3c2)n1] | 190 | 5-[3-(2-bromophenyl)- 1,2,4-oxadiazol-5-yl]- 1H-1,2,3-benzotriazole | 342.16 | 342 |
| 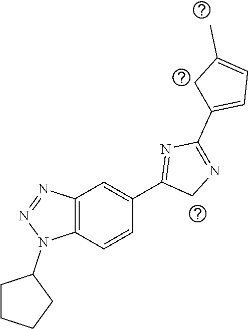 [SMILES: CC1=CC=C(C2=NCC(c3ccc4c(c3)nnn4C3CCCC3)=N2)C1] | 192 | 1-cyclopentyl-5-[3-(5- methylthiophen-2-yl)- 1,2,4-oxadiazol-5-yl]- 1H-1,2,3-benzotriazole | 351.43 | 352.1 |
| 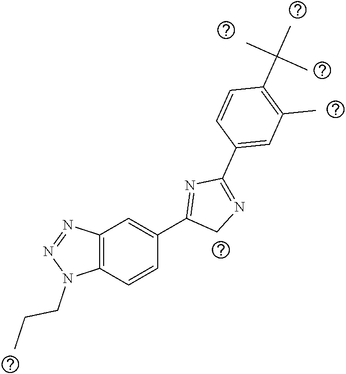 [SMILES: CCCn1nnc2cc(C3=NC(c4ccc(C(C)(C)C)c(C)c4)=NC3)ccc21] | 193 | 5-{3-[3-fluoro-4- (trifluoromethyl) phenyl]-1,2,4-oxadiazol- 5-yl}-1-propyl-1H- 1,2,3-benzotriazole | 391.33 | 392.1 |
| 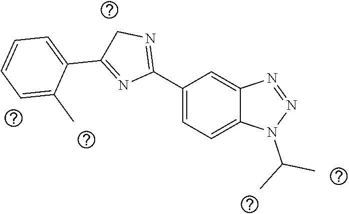 [SMILES: Cc1ccccc1C1=NC(c2ccc3c(c2)nnn3C(C)C)=NC1] | 194 | 3-(1-isopropyl-1H- benzo[d][1,2,3]triazol- 5-yl)-5-(2-methyl- pyridin-3-yl)-1,2,4- oxadiazole | 320.36 | 321 |
| 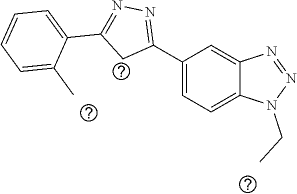 [SMILES: CCn1nnc2cc(C3=NN=C(c4ccccc4C)C3)ccc21] | 195 | 2-(5-(5-(2-bromo- phenyl)-1,2,4- oxadiazol-3-yl)-1H- benzo[d][1,2,3]triazol- 1-yl)acetic acid | 400.19 | 400 |
| 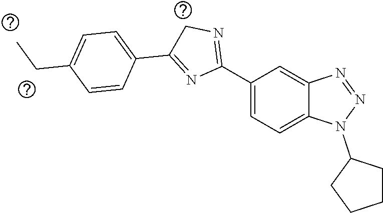 [SMILES: CCc1ccc(C2=NC(c3ccc4c(c3)nnn4C3CCCC3)=NC2)cc1] | 196 | 3-(1-cyclopentyl- 1H-benzo[d][1,2,3] triazol-5-yl)-5-(4- methoxyphenyl)- 1,2,4-oxadiazole | 361.41 | 362 |
| 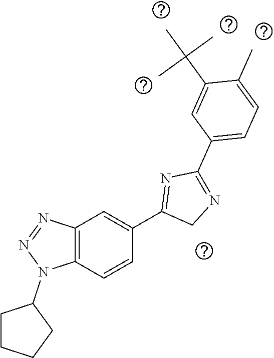 [SMILES: Cc1ccc(C2=NCC(c3ccc4c(c3)nnn4C3CCCC3)=N2)cc1C(C)(C)C] | 197 | 1-cyclopentyl-5-{3- [4-methoxy-3- (trifluoromethyl) phenyl]-1,2,4- oxadiazol-5-yl}-1H- 1,2,3-benzotriazole | 429.40 | 430.1 |
| 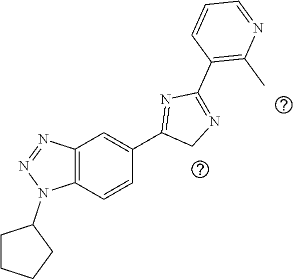 [SMILES: Cc1ncccc1C1=NCC(c2ccc3c(c2)nnn3C2CCCC2)=N1] | 198 | 5-[3-(2-chloro- pyridin-3-yl)-1,2,4- oxadiazol-5-yl]-1- cyclopenty1-1H- 1,2,3-benzotriazole | 366.81 | 367.1 |
| 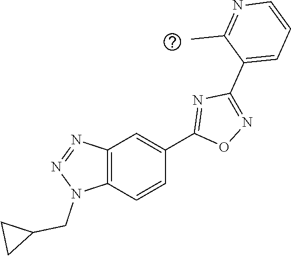 [SMILES: Cc1ncccc1-c1noc(-c2ccc3c(c2)nnn3CC2CC2)n1] | 199 | 5-[3-(2-chloropyridin- 3-yl)-1,2,4-oxadiazol- 5-yl]-1-(cyclopropyl- methyl)-1H-1,2,3- benzotriazole | 352.78 | 353.1 |
| 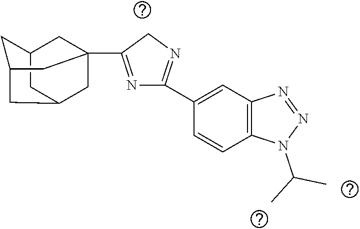 [SMILES: CC(C)n1nnc2cc(C3=NCC(C45CC6CC(CC(C6)C4)C5)=N3)ccc21] | 200 | 5-[5-(adamantan-1- yl)-1,2,4-oxadiazol- 3-yl]-1-(propan-2- yl)-1H-1,2,3- benzotriazole | 363.47 | 364 |
| 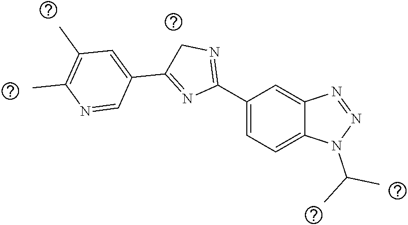 [SMILES: Cc1cc(C2=NC(c3ccc4c(c3)nnn4C(C)C)=NC2)cnc1C] | 201 | 5-(5,6-dimethyl- pyridin-3-yl)-3-(1- isopropyl-1H- benzo[d][1,2,3]triazol- 5-yl)-1,2,4- oxadiazole | 334.38 | 335.1 |
| 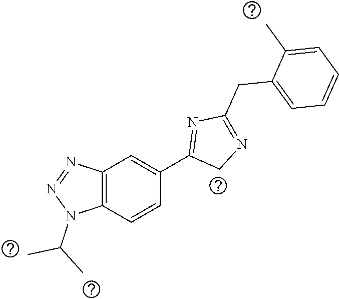 [SMILES: Cc1ccccc1CC1=NCC(c2ccc3c(c2)nnn3C(C)C)=N1] | 202 | 5-{3-[(2-methyl- phenyl)methyl]- 1,2,4-oxadiazol-5- yl}-1-(propan-2- yl)-1H-1,2,3- benzotriazole | 333.40 | 334.1 |
| 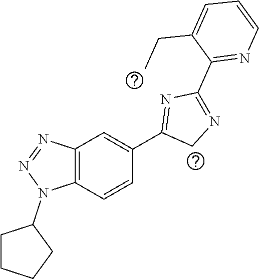 [SMILES: CCc1cccnc1C1=NCC(c2ccc3c(c2)nnn3C2CCCC2)=N1] | 203 | 1-cyclopentyl-5- [3-(3-methoxy- pyridin-2-yl)-1,2,4- oxadiazol-5-yl]-1H- 1,2,3-benzotriazole | 362.39 | 363.1 |
| 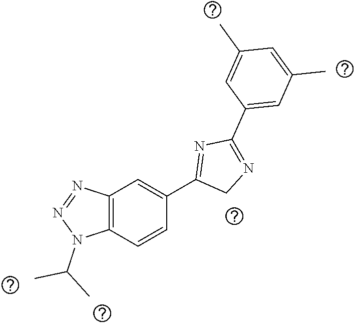 [SMILES: Cc1cc(C)cc(C2=NCC(c3ccc4c(c3)nnn4C(C)C)=N2)c1] | 204 | 5-[3-(3,5-dimethy- lphenyl)-1,2,4- oxadiazol-5-yl]-1- (propan-2-yl)- 1H-1,2,3- benzotriazole | 333.40 | 334.1 |
| 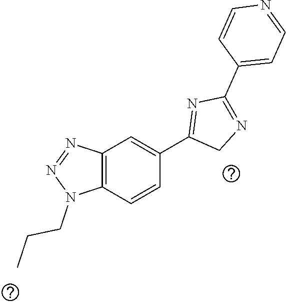 [SMILES: CCCn1nnc2cc(C3=NC(c4ccncc4)=NC3)ccc21] | 205 | 1-propyl-5-[3- (pyridin-4-yl)-1,2,4- oxadiazol-5-yl]-1H- 1,2,3- benzotriazole | 306.33 | 307.1 |
| 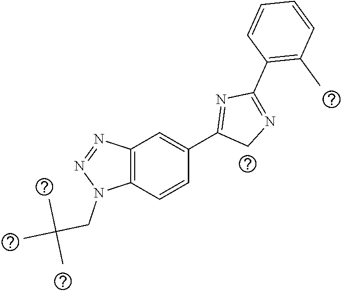 [SMILES: Cc1ccccc1C1=NCC(c2ccc3c(c2)nnn3CC(C)(C)C)=N1] | 206 | 2-methyl-1-{5-[3- (4-methylpyridin- 3-yl)-1,2,4-oxadiazol- 5-yl]-1H-1,2,3- benzotriazol-1- yl}propan-2-ol | 350.38 | 351.1 |
| 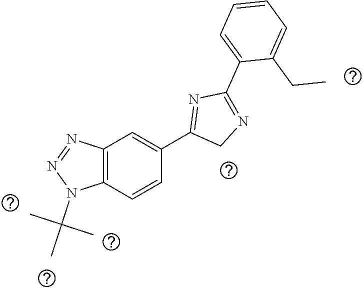 [SMILES: CCc1ccccc1C1=NCC(c2ccc3c(c2)nnn3C(C)(C)C)=N1] | 207 | 1-tert-butyl-5-[3- (2-methoxyphenyl)- 1,2,4-oxadiazol- 5-yl]-1H-1,2,3- benzotriazole | 349.39 | 350.2 |
| 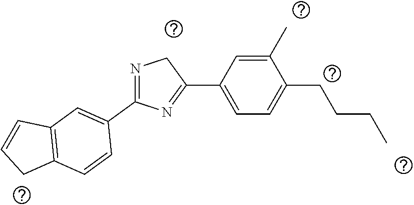 [SMILES: CCCCc1ccc(C2=NC(c3ccc4c(c3)C=CC4)=NC2)cc1C] | 208 | 5-(3-(1H-indol-5-yl)- 1,2,4-oxadiazol-5-yl)- 2-(allylamino) benzonitrile | 341.37 | 342.2 |
| 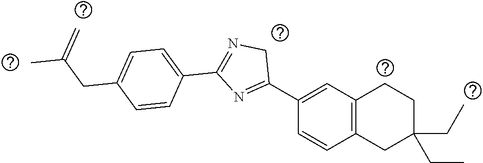 [SMILES: C=C(C)Cc1ccc(C2=NCC(c3ccc4c(c3)CCC(CC)(CC)C4)=N2)cc1] | 209 | N-(4-(5-(2,2- diethyl-4-oxochroman- 6-yl)-1,2,4-oxadiazol- 3-yl)phenyl)acetamide | 405.45 | 406.2 |
| 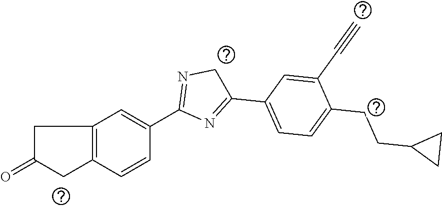 [SMILES: C#Cc1cc(C2=NC(c3ccc4c(c3)CC(=O)C4)=NC2)ccc1CCC1CC1] | 210 | 2-((cyclopropyl- methyl)amino)-5- (3-(2-oxoindolin- 5-yl)-1,2,4- oxadiazol-5- yl)benzonitrile | 371.40 | 372.1 |
| 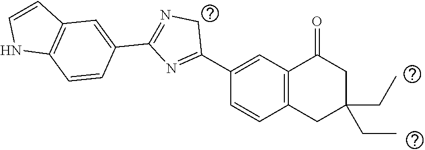 [SMILES: CCC1(CC)CC(=O)c2cc(C3=NC(c4ccc5[nH]ccc5c4)=NC3)ccc2C1] | 211 | 6-(3-(1H-benzo[d] imidazol-5-yl)- 1,2,4-oxadiazol-5-yl)- 2,2-diethylchroman- 4-one | 388.43 | 389.1 |
| 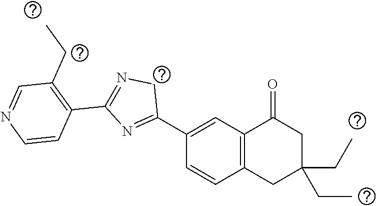 [SMILES: CCc1cnccc1C1=NCC(c2ccc3c(c2)C(=O)CC(CC)(CC)C3)=N1] | 212 | 2,2-diethyl-6-(3-(3- methoxypyridin-4-yl)- 1,2,4-oxadiazol-5- yl)chroman-4-one | 379.42 | 380.2 |
| 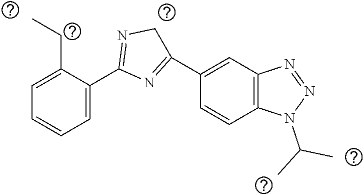 [SMILES: CCc1ccccc1C1=NCC(c2ccc3c(c2)nnn3C(C)C)=N1] | 213 | 5-[3-(2-methoxy- phenyl)-1,2,4- thiadiazol-5-yl]-1- (propan-2-yl)- 1H-1,2,3- benzotriazole | 351.43 | 352 |
| 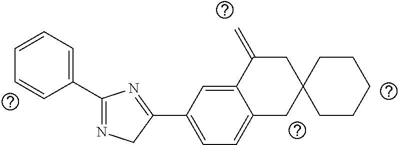 [SMILES: C=C1CC2(CCCCC2)Cc2ccc(C3=NC(c4ccccc4)=NC3)cc21] | 214 | 6-[3-(pyridin-3-yl)- 1,2,4-oxadiazol-5- yl]-3,4-dihydrospiro[1- benzopyran-2,4′- oxane]-4-one | 363.37 | 364.1 |
| 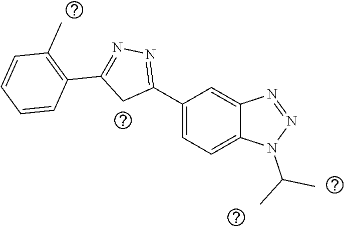 [SMILES: Cc1ccccc1C1=NN=C(c2ccc3c(c2)nnn3C(C)C)C1] | 215 | 2-(1-isopropyl-1H- benzo[d][1,2,3]triazol- 5-yl)-5-(2-(trifluoro- methyl)phenyl)thiazole | 388.41 | 389.2 |
| 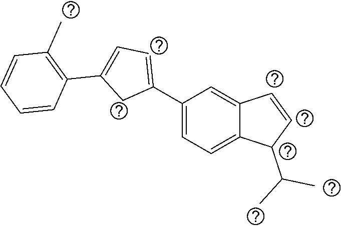 [SMILES: Cc1ccccc1C1=CC=C(c2ccc3c(c2)C=CC3C(C)C)C1] | 216 | 2-(1-isopropyl- benzotriazol-5-yl)-5- (o-tolyl)thiazole | 334.44 | 335.1 |
| 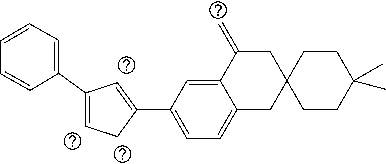 [SMILES: C=C1CC2(CCC(C)(C)CC2)Cc2ccc(C3=CC(c4ccccc4)=CC3)cc21] | 217 | 4′,4′-difluoro-6-[3- (pyridin-4-yl)- 1,2,4-oxadiazol-5-yl]- 3,4-dihydrospiro[1- benzopyran-2,1′- cyclohexane]-4-one | 397.38 | 398.2 |
| 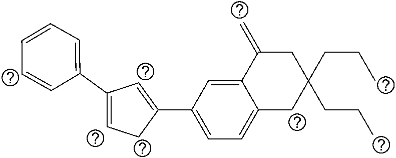 [SMILES: C=C1CC(CCC)(CCC)Cc2ccc(C3=CC(c4ccccc4)=CC3)cc21] | 218 | 2,2-dipropyl-6-[3- (pyridin-3-yl)- 1,2,4-oxadiazol-5- yl]-3,4-dihydro- 2H-1-benzopyran- 4-one | 377.44 | 378.2 |
| 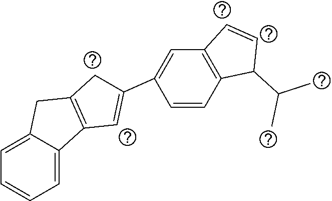 [SMILES: CC(C)C1C=Cc2cc(C3=CC4=C(C3)Cc3ccccc34)ccc21] | 219 | 5-{8H-indeno[1,2- d][1,3]oxazol-2- yl}-1-(propan-2-yl)- 1H-1,2,3-benzotriazole | 316.36 | 317 |
| 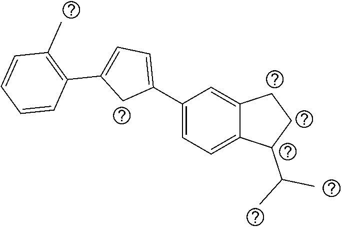 [SMILES: Cc1ccccc1C1=CC=C(c2ccc3c(c2)CCC3C(C)C)C1] | 220 | 5-(2-bromophenyl)- 2-(1-isopropylbenzo- triazol-5-yl)thiazole | 399.31 | 401 |
| 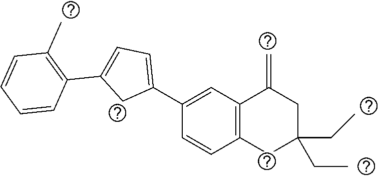 [SMILES: C=C1CC(CC)(CC)Cc2ccc(C3=CC=C(c4ccccc4C)C3)cc21] | 221 | 6-(3-(2-chlorophenyl)- 1,2,4-oxadiazol-5-yl)- 2,2-diethylchroman- 4-one | 382.84 | 383.1 |
| 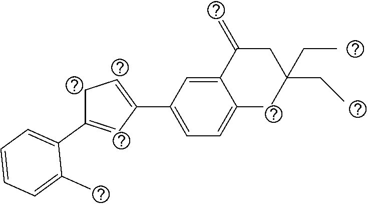 [SMILES: C=C1CC(CC)(CC)Cc2ccc(C3=CCC(c4ccccc4C)=C3)cc21] | 222 | 2,2-diethyl-6-(5-(2- methoxyphenyl)-1,2,4- thiadiazol-3- yl)chroman-4-one | 394.49 | 395.1 |
| 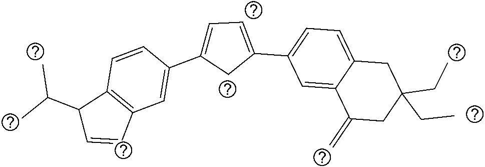 [SMILES: C=C1CC(CC)(CC)Cc2ccc(C3=CC=C(c4ccc5c(c4)C=CC5C(C)C)C3)cc21] | 223 | 2,2-diethyl-6-(5-(1- isopropyl-1H- benzo[d][1,2,3]triazol- 5-yl)-1,3,4-oxadiazol- 2-yl)chroman-4-one | 431.50 | 432.2 |
| 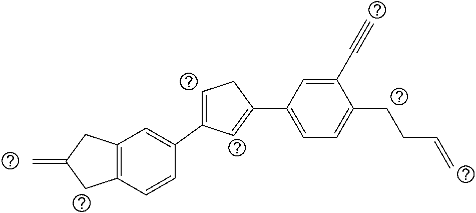 [SMILES: C#Cc1cc(C2=CC(c3ccc4c(c3)CC(=C)C4)=CC2)ccc1CCC=C] | 224 | 2-(allylamino)-5-(3- (2-oxoindolin-5- yl)-1,2,4-oxadiazol-5- yl)benzonitrile | 357.37 | 358.1 |
| 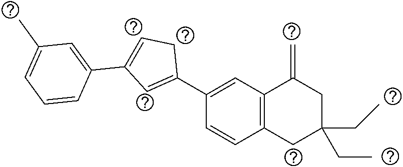 [SMILES: C=C1CC(CC)(CC)Cc2ccc(C3=CC(c4cccc(C)c4)=CC3)cc21] | 225 | 2,2-diethyl-6-(3- (m-tolyl)-1,2,4- oxadiazol-5- yl)chroman-4-one | 362.43 | 363.1 |
| 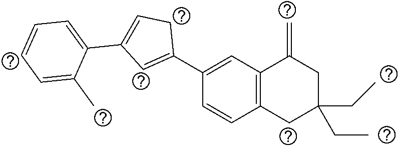 [SMILES: C=C1CC(CC)(CC)Cc2ccc(C3=CC(c4ccccc4C)=CC3)cc21] | 226 | 2,2-diethyl-6-(3- (3-fluoropyridin-4- yl)-1,2,4-oxadiazol- 5-yl)chroman-4-one | 367.38 | 368.1 |
| 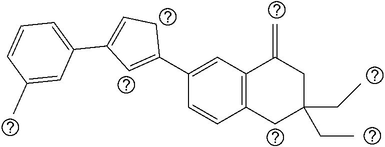 [SMILES: C=C1CC(CC)(CC)Cc2ccc(C3=CC(c4cccc(C)c4)=CC3)cc21] | 227 | 2,2-diethyl-6-[3- (3-hydroxyphenyl)- 1,2,4-oxadiazol-5- yl]chroman-4-one | 364.40 | 365.1 |
| 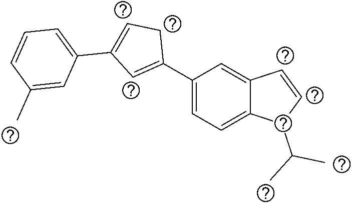 [SMILES: Cc1cccc(C2=CCC(c3ccc4c(c3)C=CC4C(C)C)=C2)c1] | 228 | 5-(1-isopropyl-1H- benzo[d][1,2,3]triazol- 5-yl)-3-(3-methoxy- phenyl)-1,2,4-oxadiazole | 335.37 | 336 |
| 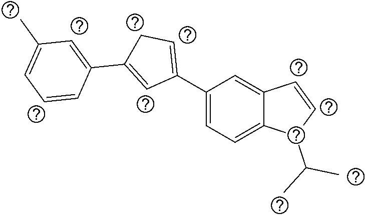 [SMILES: Cc1cccc(C2=CC(c3ccc4c(c3)C=CC4C(C)C)=CC2)c1] | 229 | 3-(1-isopropyl-1H- benzo[d][1,2,3]triazol- 5-yl)-5-(6-methyl- pyrazin-2-yl)-1,2,4- oxadiazole | 321.34 | 322.1 |
| 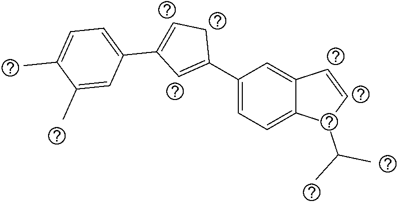 [SMILES: Cc1ccc(C2=CCC(c3ccc4c(c3)C=CC4C(C)C)=C2)cc1C] | 230 | 3-(3,4-dimethoxy- phenyl)-5-(1- isopropyl-1H- benzo[d][1,2,3]triazol- 5-yl)-1,2,4-oxadiazole | 365.39 | 366 |
| 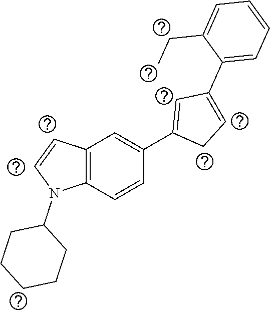 [SMILES: CCc1ccccc1C1=CCC(c2ccc3c(ccn3C3CCCCC3)c2)=C1] | 231 | 5-[3-(2-methoxy- phenyl)-1,2,4- oxadiazol-5-yl]-1- (oxan-4-yl)-1H- 1,2,3-benzotriazole | 377.40 | 378.2 |
| 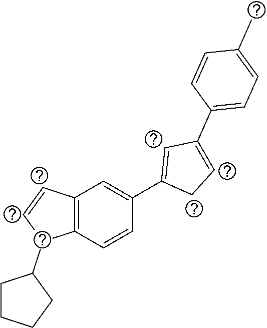 [SMILES: Cc1ccc(C2=CCC(c3ccc4c(c3)C=CC4C3CCCC3)=C2)cc1] | 232 | 1-cyclopentyl-5-[3- (4-fluorophenyl)- 1,2,4-oxadiazol-5 yl]-1H-1,2,3- benzotriazole | 349.37 | 350.2 |
| 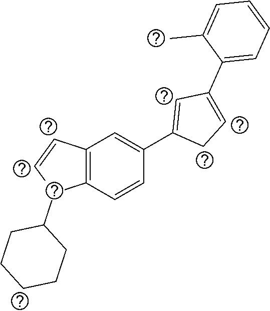 [SMILES: Cc1ccccc1C1=CCC(c2ccc3c(c2)C=CC3C2CCCCC2)=C1] | 233 | 5-[3-(2-bromophenyl)- 1,2,4-oxadiazol-5- yl]-1-(oxan-4-yl)-1H- 1,2,3-benzotriazole | 426.27 | 426.1 |
| 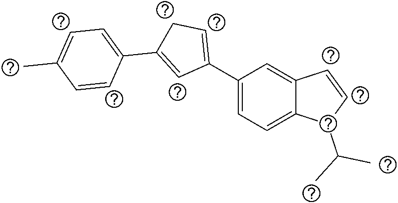 [SMILES: Cc1ccc(C2=CC(c3ccc4c(c3)C=CC4C(C)C)=CC2)cc1] | 234 | 3-(1-isopropyl-1H- benzo[d][1,2,3]triazol- 5-yl)-5-(5-methyl- pyrazin-2-yl)-1,2,4- oxadiazole | 321.34 | 322.1 |
| 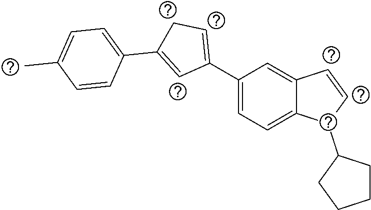 [SMILES: Cc1ccc(C2=CC(c3ccc4c(c3)C=CC4C3CCCC3)=CC2)cc1] | 235 | 3-(1-cyclopentyl-1H- benzo[d][1,2,3]triazol- 5-yl)-5-(p-tolyl)- 1,2,4-oxadiazole | 345.41 | 346 |
| 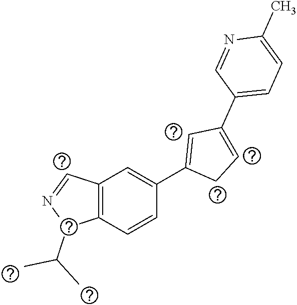 [SMILES: Cc1ccc(C2=CCC(c3ccc4c(c3)C=NC4C(C)C)=C2)cn1] | 236 | 5-[3-(6-methylpyridin- 3-yl)-1,2,4-oxadiazol- 5-yl]-1-(propan-2-yl)- 1H-1,2,3-benzotriazole | 320.36 | 321.1 |
| 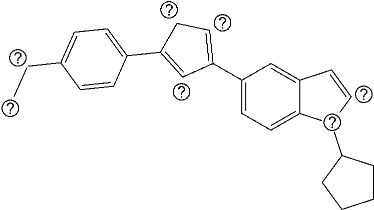 [SMILES: CCc1ccc(C2=CC(c3ccc4c(c3)C=CC4C3CCCC3)=CC2)cc1] | 237 | 3-(1-cyclopentyl-1H- benzo[d][1,2,3]triazol- 5-yl)-5-(4-(methyl- thio)phenyl)-1,2,4- oxadiazole | 377.47 | 378.1 |
| 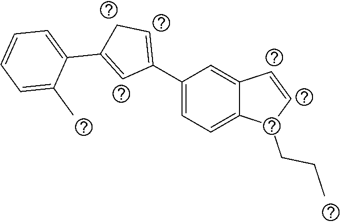 [SMILES: CCCC1C=Cc2cc(C3=CCC(c4ccccc4C)=C3)ccc21] | 238 | 2-(5-(5-(2-bromo- phenyl)-1,2,4- oxadiazol-3-yl)-1H- benzo[d][1,2,3]triazol- 1-yl)ethanol | 386.21 | 386 |
| 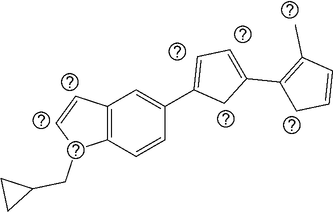 [SMILES: CC1=C(C2=CC=C(c3ccc4c(c3)C=CC4CC3CC3)C2)CC=C1] | 239 | 1-(cyclopropyl- methyl)-5-[3-(3- methylthiophen-2- yl)-1,2,4-oxadiazol- 5-yl]-1H-1,2,3- benzotriazole | 337.40 | 338.2 |
| 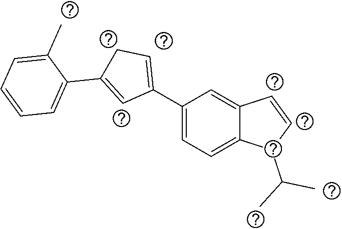 [SMILES: Cc1ccccc1C1=CC(c2ccc3c(c2)C=CC3C(C)C)=CC1] | 240 | 3-(1-isopropyl-1H- benzo[d][1,2,3]triazol- 5-yl)-5-(2-(trifluoro- methoxy)phenyl)- 1,2,4-oxadiazole | 389.34 | 390 |
| 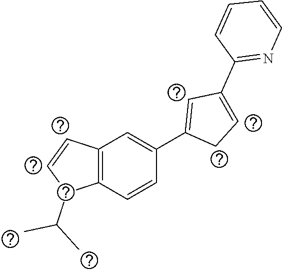 [SMILES: CC(C)C1C=Cc2cc(C3=CC(c4ccccn4)=CC3)ccc21] | 241 | 1-(propan-2-yl)-5- [3-(pyridin-2-yl)- 1,2,4-oxadiazol-5-yl]- 1H-1,2,3- benzotriazole | 306.33 | 307.2 |
| 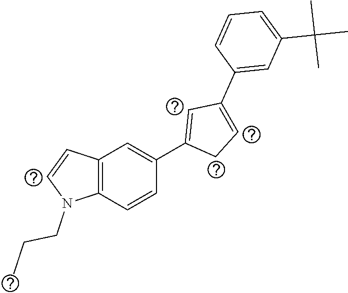 [SMILES: CCCn1ccc2cc(C3=CC(c4cccc(C(C)(C)C)c4)=CC3)ccc21] | 242 | 1-propyl-5-{3-[3- (trifluoromethyl) phenyl]-1,2,4- oxadiazol-5- yl}-1H-1,2,3- benzotriazole | 373.34 | 374.3 |
| 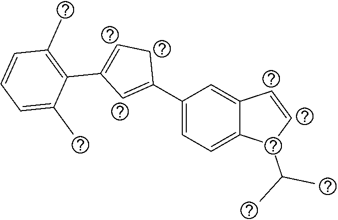 [SMILES: Cc1cccc(C)c1C1=CCC(c2ccc3c(c2)C=CC3C(C)C)=C1] | 243 | 3-(2,6-dimethyl- phenyl)-5-(1- isopropyl-1H- benzo[d][1,2,3]triazol- 5-yl)-1,2,4-oxadiazole | 333.40 | 334 |
| 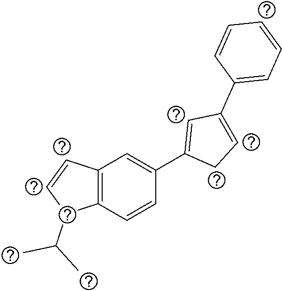 [SMILES: CC(C)C1C=Cc2cc(C3=CC(c4ccccc4)=CC3)ccc21] | 244 | 1-(propan-2-yl)-5- [3-(pyridin-4-yl)- 1,2,4-oxadiazol-5- yl]-1H-1,2,3- benzotriazole | 306.33 | 307.1 |
| 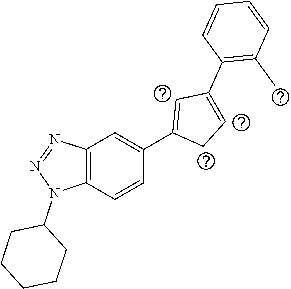 [SMILES: Cc1ccccc1C1=CCC(c2ccc3c(c2)nnn3C2CCCCC2)=C1] | 245 | 1-cyclohexyl-5-[3- (2-methylphenyl)- 1,2,4-oxadiazol-5- yl]-1H-1,2,3- benzotriazole | 359.43 | 360.2 |
| 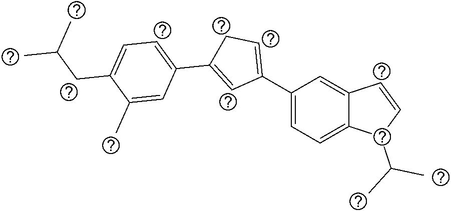 [SMILES: Cc1cc(C2=CC(c3ccc4c(c3)C=CC4C(C)C)=CC2)ccc1CC(C)C] | 246 | 5-(4-isopropoxy-3- (trifluoromethyl)phenyl)- 3-(1-isopropyl-1H- benzo[d][1,2,3]triazol- 5-yl)-1,2,4-oxadiazole | 431.42 | 432 |
| 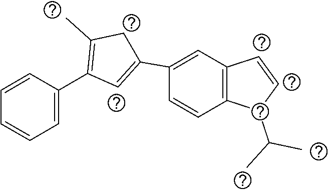 [SMILES: CC1=C(c2ccccc2)C=C(c2ccc3c(c2)C=CC3C(C)C)C1] | 247 | 5-(5-methyl-4- phenyl-1,3-oxazol- 2-yl)-1-(propan-2- yl)-1H-1,2,3- benzotriazole | 318.38 | 319 |
| 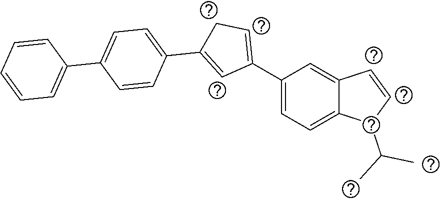 [SMILES: CC(C)C1C=Cc2cc(C3=CCC(c4ccc(-c5ccccc5)cc4)=C3)ccc21] | 248 | 5-([1,1′-biphenyl]-4- yl)-3-(1-isopropyl- 1H-benzo[d][1,2,3] triazol-5-yl)-1,2,4- oxadiazole | 381.44 | 382.1 |
| 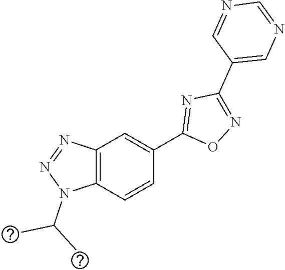 [SMILES: CC(C)n1nnc2cc(-c3nc(-c4cncnc4)no3)ccc21] | 249 | 1-(propan-2-yl)-5- [3-(pyrimidin-5- yl)-1,2,4-oxadiazol- 5-yl]-1H-1,2,3- benzotriazole | 307.32 | 308.1 |
| 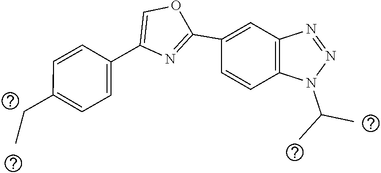 [SMILES: CCc1ccc(-c2coc(-c3ccc4c(c3)nnn4C(C)C)n2)cc1] | 250 | 5-[4-(4-methoxy- phenyl)-1,3- oxazol-2-yl]-1- (propan-2-yl)-1H- 1,2,3-benzotriazole | 334.38 | 335 |
| 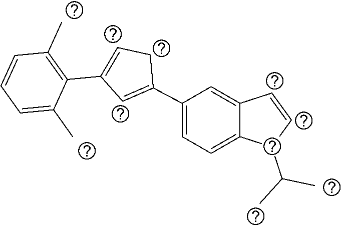 [SMILES: Cc1cccc(C)c1C1=CCC(c2ccc3c(c2)C=CC3C(C)C)=C1] | 251 | 3-(2,6-dimethoxy- phenyl)-5-(1-isopropyl- benzotriazol-5-yl)-1,2,4- oxadiazole | 365.39 | 366.1 |
| 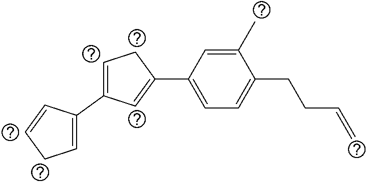 [SMILES: C=CCCc1ccc(C2=CC(C3=CCC=C3)=CC2)cc1C] | 252 | 5-(3-(1H-pyrazol-4- yl)-1,2,4-oxadiazol- 5-yl)-2-(allyl- amino)benzonitrile | 292.30 | 293.1 |
| 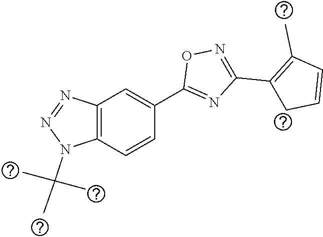 [SMILES: CC1=C(c2noc(-c3ccc4c(c3)nnn4C(C)(C)C)n2)CC=C1] | 253 | 1-tert-butyl-5-[3- (3-methylthiophen- 2-yl)-1,2,4-oxadiazol- 5-yl]-1H-1,2,3- benzotriazole | 339.42 | 340.1 |
| 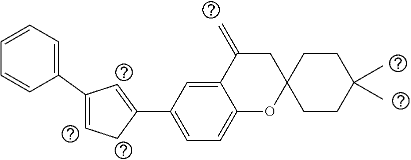 [SMILES: C=C1CC2(CCC(C)(C)CC2)Oc2ccc(C3=CC(c4ccccc4)=CC3)cc21] | 254 | 4′,4′-dimethyl-6-[3- (pyridin-4-yl)-1,2,4- oxadiazol-5-yl]-3,4- dihydrospiro[1- benzopyran-2,1′- cyclohexane]-4-one | 389.46 | 390.2 |
| 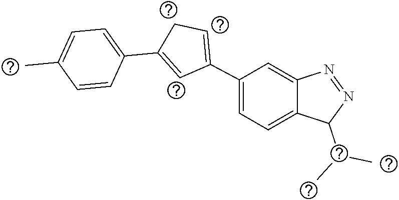 [SMILES: Cc1ccc(C2=CC(c3ccc4c(c3)N=NC4C(C)C)=CC2)cc1] | 255 | 5-(4-fluorophenyl)- 3-(1-isopropyl- benzotriazol-5-yl)- 1,2,4-oxadiazole | 323.33 | 324.1 |
| 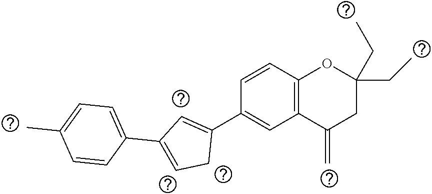 [SMILES: C=C1CC(CC)(CC)Oc2ccc(C3=CC(c4ccc(C)cc4)=CC3)cc21] | 256 | 6-(3-(4-amino- phenyl)-1,2,4- oxadiazol-5-yl)-2,2- diethylchroman-4-one | 363.42 | 364.2 |
| 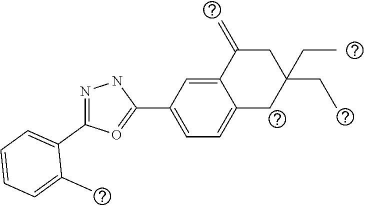 | 257 | 2,2-diethyl-6-(3-(2- methoxyphenyl)-1,2,4- thiadiazol-5- yl)chroman-4-one | 394.49 | 395.1 |
| 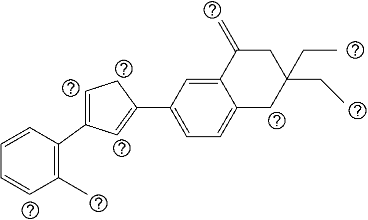 [SMILES: C=C1CC(CC)(CC)Cc2ccc(C3=CC(c4ccccc4C)=CC3)cc21] | 258 | 2,2-diethyl-6-(3-(2- hydroxypyridin- 3-yl)-1,2,4-oxadiazol-5- yl)chroman-4-one | 365.39 | 366.3 |
| 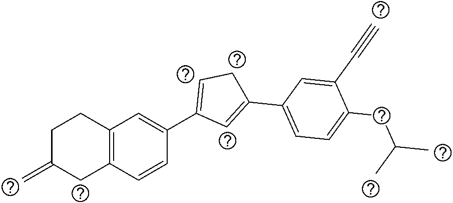 [SMILES: C#Cc1cc(C2=CC(c3ccc4c(c3)CCC(=C)C4)=CC2)ccc1CC(C)C] | 259 | 2-(isopropylamino)-5- (3-(2-oxo-1,2,3,4- tetrahydroquinolin-6- yl)-1,2,4-oxadiazol- 5-yl)benzonitrile | 373.42 | 374.1 |
| 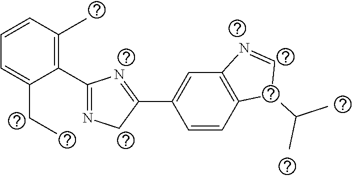 [SMILES: CCc1cccc(C)c1C1=NCC(c2ccc3c(c2)N=CC3C(C)C)=N1] | 260 | 3-(2-fluoro-6- methoxy-phenyl)-5- (1-isopropylbenzo- triazol-5-yl)- 1,2,4-oxadiazole | 353.36 | 354.1 |
| 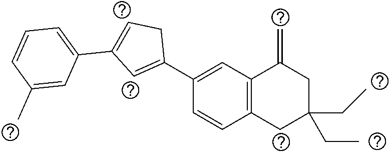 [SMILES: C=C1CC(CC)(CC)Cc2ccc(C3=CC(c4cccc(C)c4)=CC3)cc21] | 261 | 6-[3-(3-chlorophenyl)- 1,2,4-oxadiazol-5-yl]- 2,2-diethyl- chroman-4-one | 382.84 | 383.1 |
| 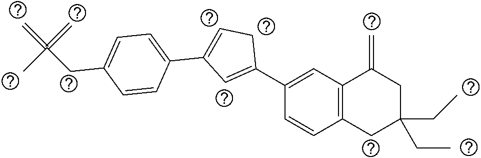 | 262 | N-(4-(5-(2,2- diethyl-4-oxochroman- 6-yl)-l,2,4-oxadiazol- 3-yl)phenyl)methane- sulfonamide | 441.50 | 442 |
| 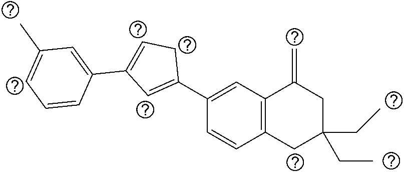 [SMILES: C=C1CC(CC)(CC)Cc2ccc(C3=CC(c4cccc(C)c4)=CC3)cc21] | 263 | 2,2-diethyl-6-[3- (2-hydroxy-4- pyridyl)-1,2,4-oxadiazol- 5-yl]chroman-4-one | 365.39 | 366.1 |
| 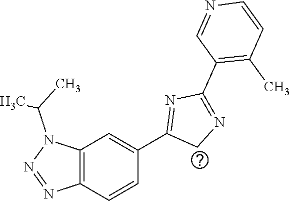 [SMILES: Cc1ccncc1C1=NCC(c2ccc3nnn(C(C)C)c3c2)=N1] | 264 | 6-[3-(4-methylpyridin- 3-yl)-1,2,4-oxadiazol- 5-yl]-1-(propan-2-yl)- 1H-1,2,3-benzotriazole | 320.36 | 321.1 |
| 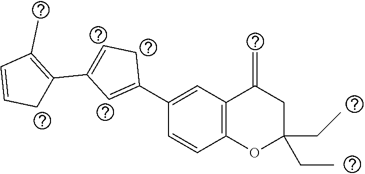 [SMILES: C=C1CC(CC)(CC)Oc2ccc(C3=CC(C4=C(C)C=CC4)=CC3)cc21] | 265 | 2,2-diethyl-6-(3-(3- methylthiophen-2- yl)-1,2,4-oxadiazol-5- yl)chroman-4-one | 368.45 | 369.1 |
| 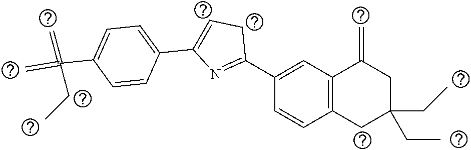 | 266 | 4-(5-(2,2-diethyl-4- oxochroman-6- yl)-1,2,4-oxadiazol-3- yl)-N-methylbenzene- sulfonamide | 441.50 | 442.1 |
| 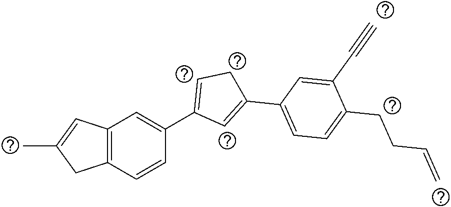 [SMILES: C#Cc1cc(C2=CC(c3ccc4c(c3)C=C(C)C4)=CC2)ccc1CCC=C] | 267 | 2-(allylamino)-5-(3- (2-aminobenzo[d] thiazol-5-yl)-1,2,4- oxadiazol-5- yl)benzonitrile | 374.42 | 375.3 |
| 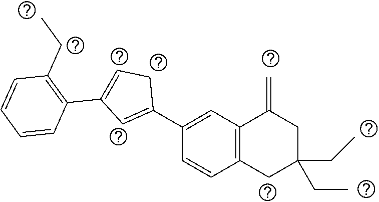 [SMILES: C=C1CC(CC)(CC)Cc2ccc(C3=CC(c4ccccc4CC)=CC3)cc21] | 268 | 2,2-diethyl-6-[3-(2- methoxyphenyl)-1,2,4- oxadiazol-5- yl]chroman-4-one | 378.43 | 379.1 |
| 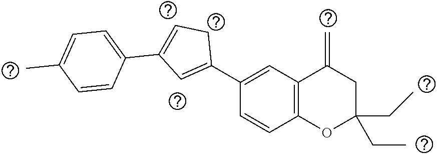 [SMILES: C=C1CC(CC)(CC)Oc2ccc(C3=CC(c4ccc(C)cc4)=CC3)cc21] | 269 | 2,2-diethyl-6-[3-(p- tolyl)-1,2,4-oxadiazol- 5-yl]chroman-4-one | 362.43 | 363.1 |
| 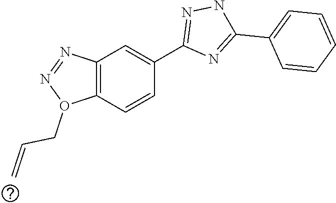 | 270 | 5-(3-phenyl-1,2,4- oxadiazol-5-yl)-1- (prop-2-en-1-yl)-1H- 1,2,3-benzotriazole | 303.33 | |
| 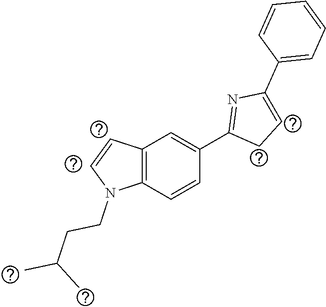 [SMILES: CC(C)CCn1ccc2cc(C3=NC(c4ccccc4)=CC3)ccc21] | 271 | 1-(3-methylbutyl)-5- (3-phenyl-1,2,4- oxadiazol-5-yl)-1H- 1,2,3-benzotriazole | 333.40 | 334.2 |
| 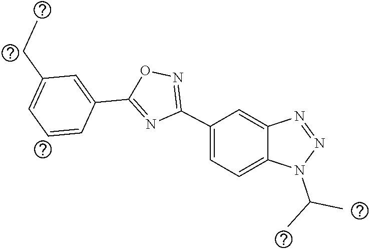 [SMILES: CCc1cccc(-c2nc(-c3ccc4c(c3)nnn4C(C)C)no2)c1] | 272 | 3-(1-isopropyl-1H- benzo[d][1,2,3]triazol- 5-yl)-5-(5-methoxy- pyridin-3-yl)-1,2,4- oxadiazole | 336.36 | 337.1 |
| 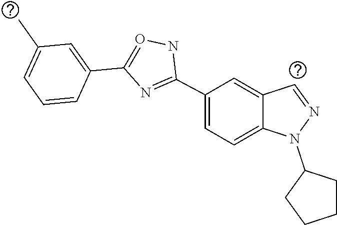 | 273 | 3-(1-cyclopentyl-1H- benzo[d][1,2,3]triazol- 5-yl)-5-(m-tolyl)- 1,2,4-oxadiazole | 345.41 | 346 |
| 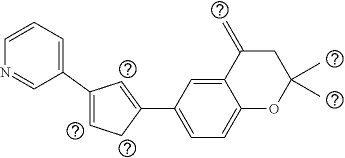 [SMILES: C=C1CC(C)(C)Oc2ccc(C3=CC(c4cccnc4)=CC3)cc21] | 274 | 2,2-dimethyl-6-[3- (pyridin-3-yl)-1,2,4- oxadiazol-5-yl]-3,4- dihydro-2H-1- benzopyran-4-one | 321.34 | 322.1 |
| 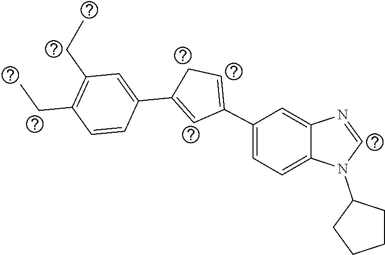 [SMILES: CCc1ccc(C2=CC(c3ccc4c(c3)ncn4C3CCCC3)=CC2)cc1CC] | 275 | 3-(1-cyclopentyl- 1H-benzo[d][1,2,3] triazol-5-yl)-5-(3,4- dimethoxyphenyl)- 1,2,4-oxadiazole | 391.43 | 392.1 |
| 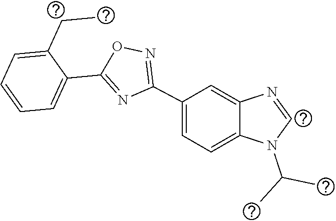 [SMILES: CCc1ccccc1-c1nc(-c2ccc3c(c2)ncn3C(C)C)no1] | 276 | 3-(1-isopropyl-1H- benzo[d][1,2,3]triazol- 5-yl)-5-(2- (methylthio)phenyl)- 1,2,4-oxadiazole | 351.43 | 352.1 |
| 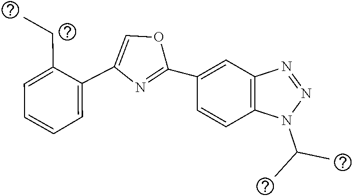 [SMILES: CCc1ccccc1-c1coc(-c2ccc3c(c2)nnn3C(C)C)n1] | 277 | 5-[4-(2-methoxy- phenyl)-1,3-oxazol- 2-yl]-1-(propan-2-yl)- 1H-1,2,3- benzotriazole | 334.38 | 335 |
| 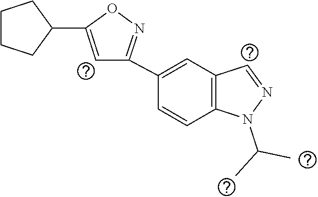 [SMILES: CC(C)n1ncc2cc(-c3cc(C4CCCC4)on3)ccc21] | 278 | 5-(5-cyclopentyl- 1,2,4-oxadiazol-3-yl)- 1-(propan-2-yl)-1H- 1,2,3-benzotriazole5- (5-cyclopentyl-1,2,4- oxadiazol-3-yl)-1- (propan-2-yl)-1H- 1,2,3-benzotriazole | 297.36 | 298 |
| 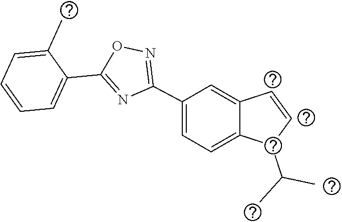 [SMILES: Cc1ccccc1-c1nc(-c2ccc3c(c2)C=CC3C(C)C)no1] | 279 | 2-(3-(1-isopropyl-1H- benzo[d][1,2,3]triazol- 5-yl)-1,2,4-oxadiazol- 5-yl)benzonitrile | 330.35 | 331 |
| 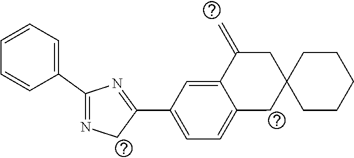 [SMILES: C=C1CC2(CCCCC2)Cc2ccc(C3=NC(c4ccccc4)=NC3)cc21] | 280 | 6-[3-(pyridin-4-yl)- 1,2,4-oxadiazol-5- yl]-3,4-dihydrospiro[1- benzopyran-2,1′- cyclohexane]-4-one | 361.40 | 362.1 |
| 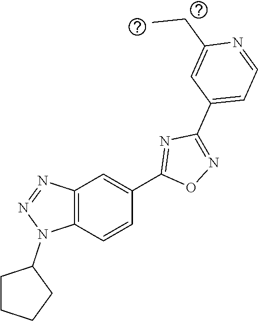 [SMILES: CCc1cc(-c2noc(-c3ccc4c(c3)nnn4C3CCCC3)n2)ccn1] | 281 | 1-cyclopentyl-5-[3-(2- methoxypyridin-4-yl)- 1,2,4-oxadiazol-5-yl]- 1H-1,2,3-benzotriazole | 362.39 | 363.1 |
| 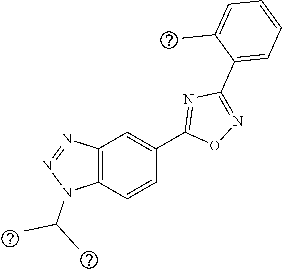 [SMILES: Cc1ccccc1-c1noc(-c2ccc3c(c2)nnn3C(C)C)n1] | 282 | 5-[3-(2-bromophenyl)- 1,2,4-oxadiazol-5-yl]- 1-(propan-2-yl)-1H- 1,2,3-benzotriazole | 384.24 | 384.2 |
| 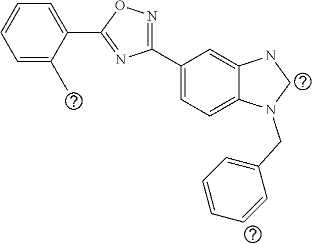 [SMILES: Cc1ccccc1-c1nc(-c2ccc3c(c2)NCN3Cc2ccccc2)no1] | 283 | 5-(2-bromophenyl)-3- (1-(pyridin-3- ylmethyl)-1H- benzo[d][1,2,3]triazol- 5-yl)-1,2,4-oxadiazole | 433.27 | 434.9 |
| 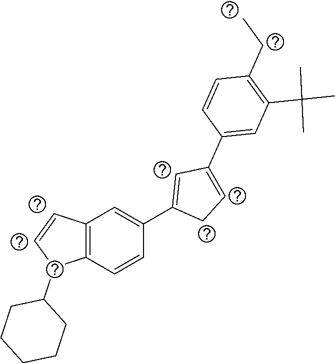 [SMILES: CCc1ccc(C2=CCC(c3ccc4c(c3)C=CC4C3CCCCC3)=C2)cc1C(C)(C)C] | 284 | 1-cyclohexyl-5-{3- [4-methoxy-3-(trifluoro- methyl)phenyl]-1,2,4- oxadiazol-5-yl}-1H- 1,2,3-benzotriazole | 443.43 | 444.2 |
| 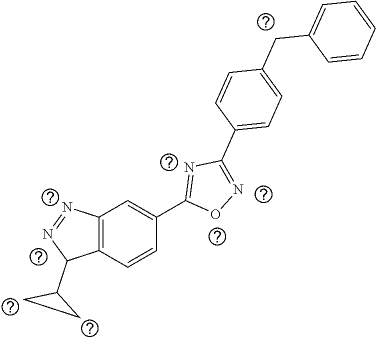 [SMILES: c1ccc(Cc2ccc(-c3noc(-c4ccc5c(c4)N=NC5C4CC4)n3)cc2)cc1] | 285 | 1-cyclopropyl-5- [3-(4-phenoxyphenyl)- 1,2,4-oxadiazol-5- yl]-1H-1,2,3- benzotriazole | 395.42 | 396.2 |
| 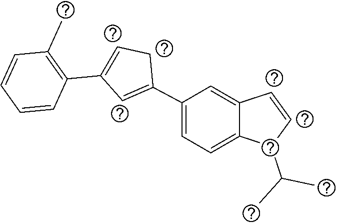 [SMILES: Cc1ccccc1C1=CCC(c2ccc3c(c2)C=CC3C(C)C)=C1] | 286 | 5-(1-isopropyl-1H- benzo[d][1,2,3]triazol- 5-yl)-3-(2-methoxy- phenyl)-1,2,4- oxadiazole | 335.37 | 356 |
| 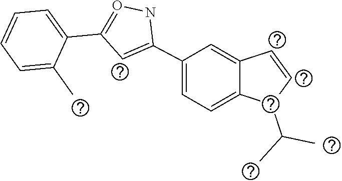 | 287 | 3-(1-isopropyl-1H- benzo[d][1,2,3]triazol- 5-yl)-5-(2-methoxy- phenyl)-1,2,4- oxadiazole | 335.37 | 356 |
| 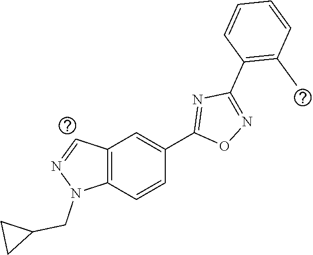 [SMILES: Cc1ccccc1-c1noc(-c2ccc3c(cnn3CC3CC3)c2)n1] | 288 | 1-(cyclopropylmethyl)- 5-[3-(2-methylphenyl)- 1,2,4-oxadiazol-5-yl]- 1H-1,2,3-benzotriazole | 331.38 | 332.2 |
| 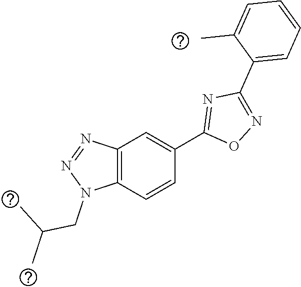 [SMILES: Cc1ccccc1-c1noc(-c2ccc3c(c2)nnn3CC(C)C)n1] | 289 | 5-[3-(2-chlorophenyl)- 1,2,4-oxadiazol-5-yl]- 1-(2-methylpropyl)-1H- 1,2,3-benzotriazole | 353.81 | 354.2 |
| 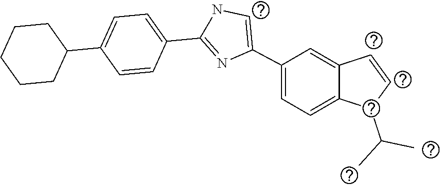 [SMILES: CC(C)C1C=Cc2cc(-c3c[nH]c(-c4ccc(C5CCCCC5)cc4)n3)ccc21] | 290 | 5-(4-cyclohexylphenyl)- 3-(1-isopropyl-1H- benzo[d][1,2,3]triazol-5- yl)-1,2,4-oxadiazole | 387.49 | 388.1 |
| 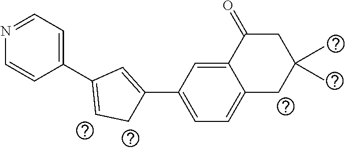 [SMILES: CC1(C)CC(=O)c2cc(C3=CC(c4ccncc4)=CC3)ccc2C1] | 291 | 2,2-dimethyl-6-[3- (pyridin-4-yl)- 1,2,4-oxadiazol-5-yl]- 3,4-dihydro-2H-1- benzopy ran-4-one | 321.34 | 322.1 |
| 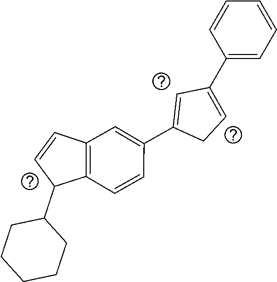 [SMILES: C1=CC(C2CCCCC2)c2ccc(C3=CC(c4ccccc4)=CC3)cc21] | 292 | 1-cyclohexyl-5-(3-phenyl- 1,2,4-oxadiazol-5-yl)- 1H-1,2,3-benzotriazole | 345.41 | 346.3 |
| 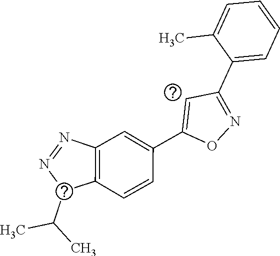 [SMILES: Cc1ccccc1-c1cc(-c2ccc3c(c2)N=NC3C(C)C)on1] | 293 | 5-[3-(2-methylphenyl)- 1,2,4-oxadiazol-5-yl]- 1-(propan-2-yl)-1H- 1,2,3-benzotriazole | 319.37 | 320.3 |
| 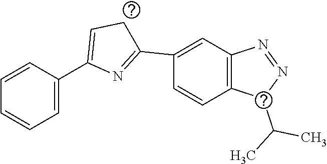 [SMILES: CC(C)C1N=Nc2cc(C3=NC(c4ccccc4)=CC3)ccc21] | 294 | 5-(4-phenyl-1,3-oxazol-2- yl)-1-(propan-2-yl)- 1H-1,2,3-benzotriazole | 304.35 | 305 |
| 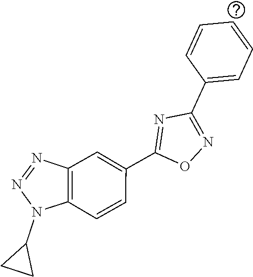 [SMILES: c1ccc(-c2noc(-c3ccc4c(c3)nnn4C3CC3)n2)cc1] | 295 | 1-cyclopropyl-5-[3- (pyridin-4-yl)-1,2,4- oxadiazol-5-yl]-1H- 1,2,3-benzotriazole | 304.31 | 305.2 |
| 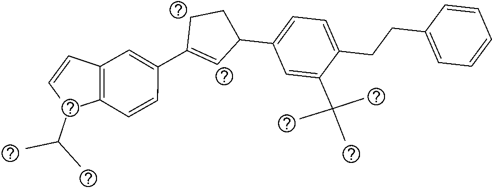 [SMILES: CC(C)C1C=Cc2cc(C3=CC(c4ccc(CCc5ccccc5)c(C(C)(C)C)c4)CC3)ccc21] | 296 | 5-{3-[4-(benzyloxy)- 3-(trifluoromethyl) phenyl]-1,2,4- oxadiazol-5-yl}-1- (propan-2-yl)- 1H-1,2,3-benzotriazole | 479.46 | 480.2 |
| 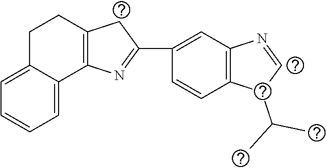 [SMILES: CC(C)C1C=Nc2cc(C3=NC4=C(CCc5ccccc54)C3)ccc21] | 297 | 5-{4H,5H-naphtho[2,1- d][1,3]oxazol-2-yl}- 1-(propan-2-yl)- 1H-1,2,3-benzotriazole | 330.39 | 331 |
| 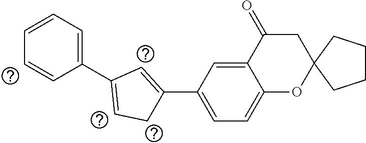 [SMILES: O=C1CC2(CCCC2)Oc2ccc(C3=CC(c4ccccc4)=CC3)cc21] | 298 | 6-[3-(pyridin-3-yl)- 1,2,4-oxadiazol-5- yl]-3,4-dihydrospiro[1- benzopyran-2,1′- cyclopentane]-4-one | 347.37 | 348.1 |
| 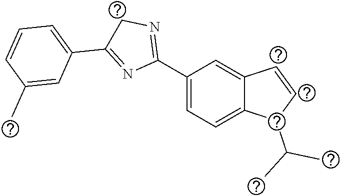 [SMILES: Cc1cccc(C2=NC(c3ccc4c(c3)C=CC4C(C)C)=NC2)c1] | 299 | 3-(1-isopropylbenzo- triazol-5-yl)-5-(3- methoxyphenyl)-1,2,4- oxadiazole | 335.37 | 336.1 |
| 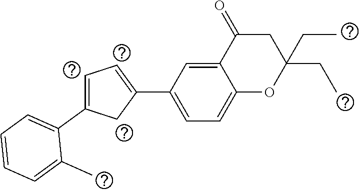 [SMILES: CCC1(CC)CC(=O)c2cc(C3=CC=C(c4ccccc4C)C3)ccc2O1] | 300 | 2,2-diethyl-6-(5-(2- methoxyphenyl)-1,3,4- oxadiazol-2- yl)chroman-4-one | 378.43 | 379.3 |
| 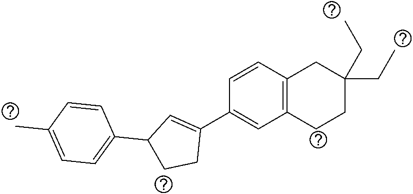 [SMILES: CCC1(CC)CCc2cc(C3=CC(c4ccc(C)cc4)CC3)ccc2C1] | 301 | 2,2-diethyl-6-[3-(6- methoxy-3-pyridyl)- 1,2,4-oxadiazol-5- yl]chroman-4-one | 379.42 | 380.3 |
| 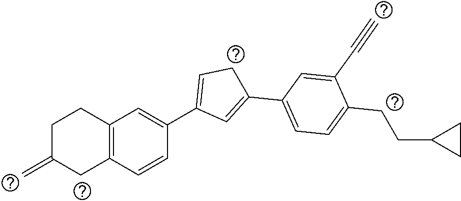 [SMILES: C#Cc1cc(C2=CC(c3ccc4c(c3)CCC(=C)C4)=CC2)ccc1CCC1CC1] | 302 | 2-((cyclopropyl- methyl)amino)-5- (3-(2-oxo-1,2,3,4- tetrahydroquinolin-6- yl)-1,2,4-oxadiazol- 5-yl)benzonitrile | 385.43 | 386.2 |
| 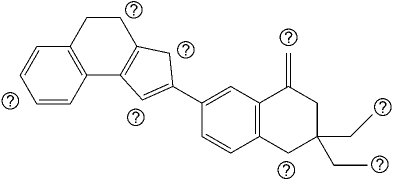 [SMILES: C=C1CC(CC)(CC)Cc2ccc(C3=CC4=C(CCc5ccccc54)C3)cc21] | 303 | 2,2-diethyl-6-(3-(4- methoxypyridin-3-yl)- 1,2,4-oxadiazol-5- yl)chroman-4-one | 379.42 | 380.3 |
| 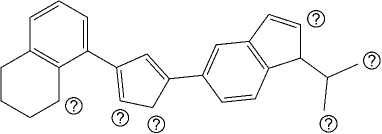 [SMILES: CC(C)C1C=Cc2cc(C3=CC(c4cccc5c4CCCC5)=CC3)ccc21] | 304 | 3-chroman-8-yl-5-(1- isopropylbenzotriazol-5- yl)-1,2,4-oxadiazole | 361.41 | 362.1 |
| 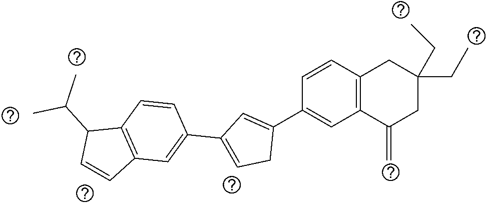 [SMILES: C=C1CC(CC)(CC)Cc2ccc(C3=CC(c4ccc5c(c4)C=CC5C(C)C)=CC3)cc21] | 305 | 2,2-diethyl-6-(3-(1- isopropyl-1H- benzo[d][1,2,3]triazol- 5-yl)-1,2,4-oxadiazol- 5-yl)chroman-4-one | 431.50 | 432.2 |
| 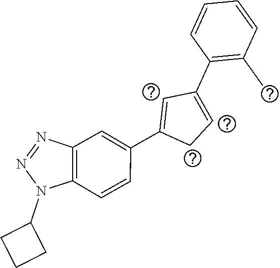 [SMILES: Cc1ccccc1C1=CCC(c2ccc3c(c2)nnn3C2CCC2)=C1] | 306 | 1-cyclobutyl-5-[3-(2- methylphenyl)-1,2,4- oxadiazol-5-yl]-1H- 1,2,3-benzotriazole | 331.38 | 332.1 |
| 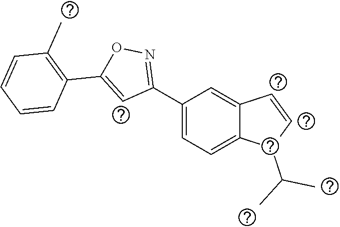 [SMILES: Cc1ccccc1-c1cc(-c2ccc3c(c2)C=CC3C(C)C)no1] | 307 | 5-(2-fluorophenyl)- 3-(1-isopropyl-1H- benzo[d][1,2,3]triazol- 5-yl)-1,2,4-oxadiazole | 323.33 | 324 |
| 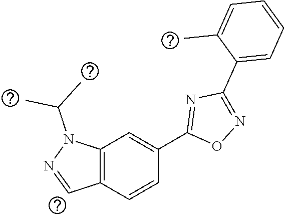 [SMILES: Cc1ccccc1-c1noc(-c2ccc3cnn(C(C)C)c3c2)n1] | 308 | 6-[3-(2-methylphenyl)- 1,2,4-oxadiazol-5- yl]-1-(propan-2-yl)- 1H-1,2,3-benzotriazole | 319.37 | 320.1 |
| 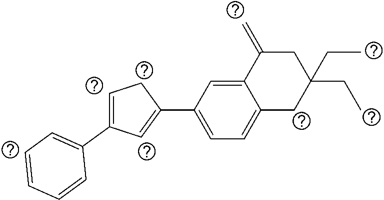 [SMILES: C=C1CC(CC)(CC)Cc2ccc(C3=CC(c4ccccc4)=CC3)cc21] | 309 | 2,2-diethyl-6-(3-(pyridin- 3-yl)-1,2,4-thiadiazol- 5-yl)chroman-4-one | 365.45 | 366 |
| 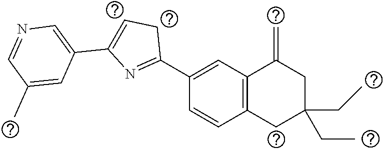 [SMILES: C=C1CC(CC)(CC)Cc2ccc(C3=NC(c4cncc(C)c4)=CC3)cc21] | 310 | 2,2-diethyl-6-[3-(5- hydroxy-3-pyridyl)-1,2,4- oxadiazol-5-yl]chroman- 4-one | 365.39 | 366.1 |
| 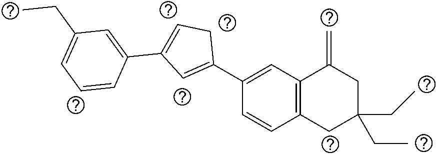 [SMILES: C=C1CC(CC)(CC)Cc2ccc(C3=CC(c4cccc(CC)c4)=CC3)cc21] | 311 | 2,2-diethyl-6-(3-(5- methoxypyridin-3-yl)- 1,2,4-oxadiazol-5- yl)chroman-4-one | 379.42 | 380.1 |
| 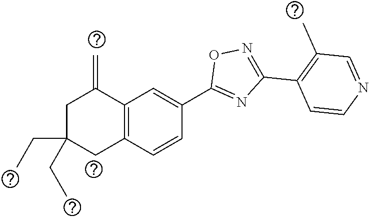 [SMILES: C=C1CC(CC)(CC)Cc2ccc(-c3nc(-c4ccncc4C)no3)cc21] | 312 | 2,2-diethyl-6-(3-(3- hydroxypyridin-4-yl)- 1,2,4-oxadiazol-5- yl)chroman-4-one | 365.39 | 366 |
| 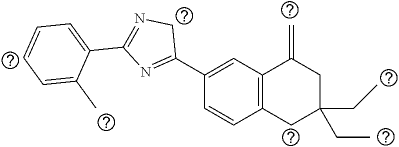 [SMILES: C=C1CC(CC)(CC)Cc2ccc(C3=NC(c4ccccc4C)=NC3)cc21] | 313 | 2,2-diethyl-6-(3-(3- (trifluoromethyl)pyridin- 4-yl)-1,2,4-oxadiazol- 5-yl)chroman-4-one | 417.39 | 418 |
| 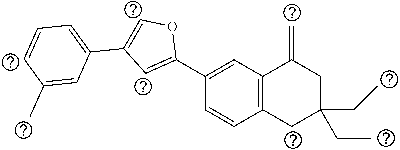 [SMILES: C=C1CC(CC)(CC)Cc2ccc(-c3cc(-c4cccc(C)c4)co3)cc21] | 314 | 2,2-diethyl-6-(3-(2- (trifluoromethyl)pyridin- 4-yl)-1,2,4-oxadiazol- 5-yl)chroman-4-one | 417.39 | 418 |
| 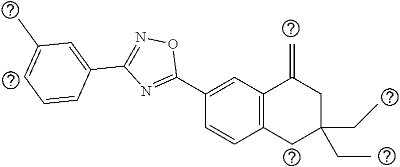 [SMILES: C=C1CC(CC)(CC)Cc2ccc(-c3nc(-c4cccc(C)c4)no3)cc21] | 315 | 2,2-diethyl-6-(3-(2- methylpyridin-4-yl)- 1,2,4-oxadiazol-5- yl)chroman-4-one | 363.42 | 364.1 |
| 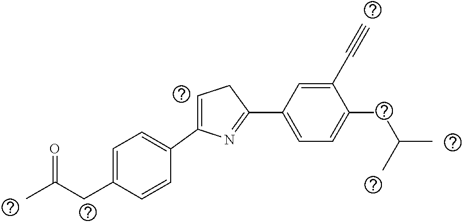 [SMILES: C#Cc1cc(C2=NC(c3ccc(CC(C)=O)cc3)=CC2)ccc1CC(C)C] | 316 | N-(4-(5-(3-cyano-4- (isopropylamino)phenyl)- 1,2,4-oxadiazol-3- yl)phenyl)acetamide | 361.41 | 362.1 |
| 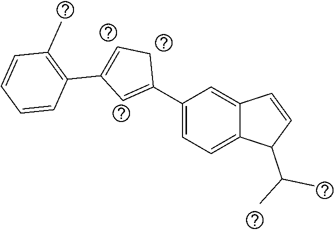 [SMILES: Cc1ccccc1C1=CCC(c2ccc3c(c2)C=CC3C(C)C)=C1] | 320 | 3-(2-chlorophenyl)-5- (1-isopropyl-1H- indol-5-yl)-1,2,4- oxadiazole | 337.81 | 338 |
| 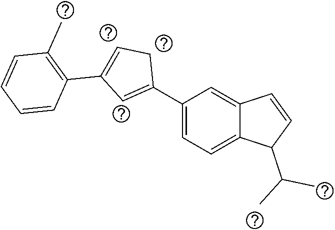 [SMILES: Cc1ccccc1C1=CCC(c2ccc3c(c2)C=CC3C(C)C)=C1] | 321 | 5-(1-isopropyl-1H-indol- 5-yl)-3-(2-(trifluoro- methoxy)phenyl)-1,2,4- oxadiazole | 387.36 | 388.1 |
| 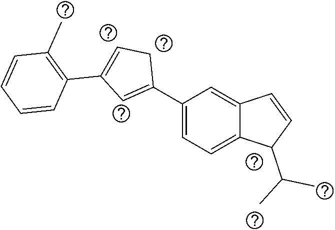 [SMILES: Cc1ccccc1C1=CCC(c2ccc3c(c2)C=CC3C(C)C)=C1] | 322 | 3-(2-bromophenyl)-5- (1-isopropyl-1H-indol- 5-yl)-1,2,4-oxadiazole | 382.26 | 384 |
| 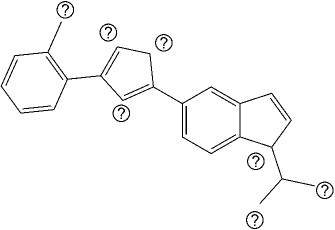 [SMILES: Cc1ccccc1C1=CCC(c2ccc3c(c2)C=CC3C(C)C)=C1] | 323 | 2-(5-(1-isopropyl-1H- indol-5-yl)-1,2,4- oxadiazol-3-yl)phenol | 319.36 | 320.1 |
| 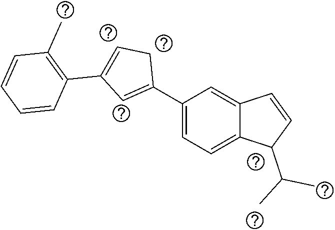 [SMILES: Cc1ccccc1C1=CCC(c2ccc3c(c2)C=CC3C(C)C)=C1] | 324 | 3-(2-isopropoxyphenyl)- 5-(1-isopropyl-1H- indol-5-yl)-1,2,4- oxadiazole | 361.45 | 362.1 |
| 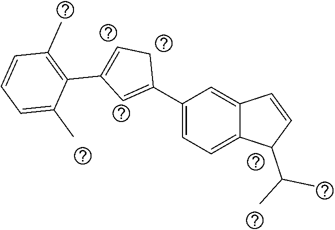 [SMILES: Cc1cccc(C)c1C1=CCC(c2ccc3c(c2)C=CC3C(C)C)=C1] | 325 | 3-(2,6-dimethoxy- phenyl)-5-(1-isopropyl- indol-5-yl)-1,2,4- oxadiazole | 363.42 | 364.1 |
| 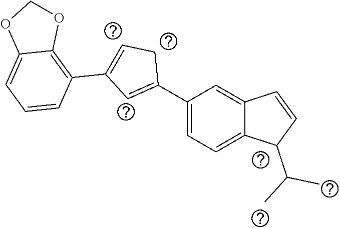 [SMILES: CC(C)C1C=Cc2cc(C3=CC(c4cccc5c4OCO5)=CC3)ccc21] | 326 | 3-(benzo[d][1,3]dioxol- 4-yl)-5-(1-isopropyl- 1H-indol-5-yl)-1,2,4- oxadiazole | 347.37 | 348.1 |
| 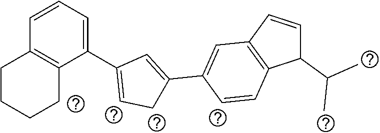 [SMILES: CC(C)C1C=Cc2cc(C3=CC(c4cccc5c4CCCC5)=CC3)ccc21] | 327 | 3-chroman-8-yl-5-(1- isopropylindol-5-yl)- 1,2,4-oxadiazole | 359.43 | 360.1 |
| 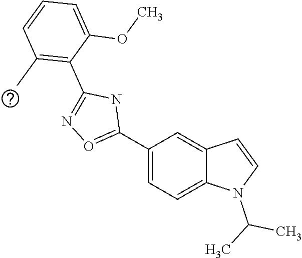 | 328 | 3-(2-fluoro-6-methoxy- phenyl)-5-(1-isopropyl- indol-5-yl)-1,2,4- oxadiazole | 351.38 | 352.1 |
| 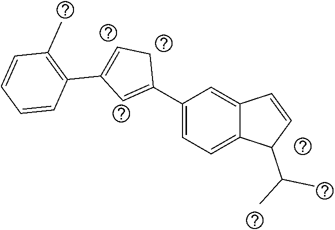 [SMILES: Cc1ccccc1C1=CCC(c2ccc3c(c2)C=CC3C(C)C)=C1] | 329 | 5-(1-isopropyl-1H- indazol-5-yl)-3-(o- tolyl)-1,2,4-oxadiazole | 318.38 | 319.1 |
| 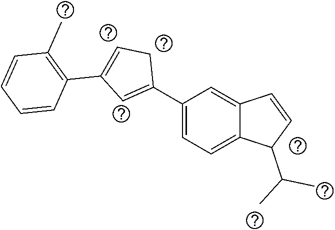 [SMILES: Cc1ccccc1C1=CCC(c2ccc3c(c2)C=CC3C(C)C)=C1] | 330 | 3-(2-chlorophenyl)- 5-(1-isopropyl- 1H-indazol-5-yl)- 1,2,4-oxadiazole | 338.80 | 339 |
| 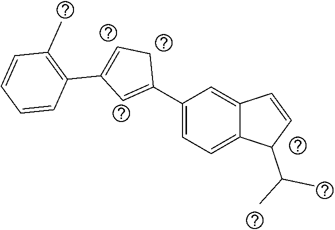 [SMILES: Cc1ccccc1C1=CCC(c2ccc3c(c2)C=CC3C(C)C)=C1] | 331 | 5-(1-isopropyl-1H- indazol-5-yl)-3- (2-(trifluoro- methoxy)phenyl)- 1,2,4-oxadiazole | 388.35 | 389.1 |
| 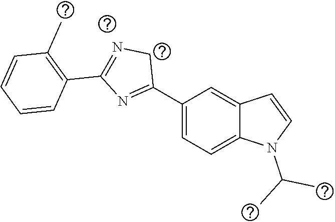 [SMILES: Cc1ccccc1C1=NCC(c2ccc3c(ccn3C(C)C)c2)=N1] | 332 | 3-(2-bromophenyl)- 5-(1-isopropyl- 1H-indazol-5-yl)- 1,2,4-oxadiazole | 383.25 | 385.1 |
| 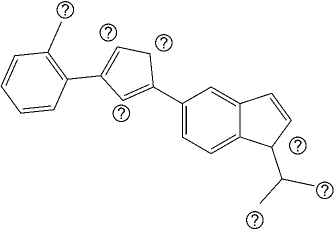 [SMILES: Cc1ccccc1C1=CCC(c2ccc3c(c2)C=CC3C(C)C)=C1] | 333 | 2-(5-(1-isopropyl- 1H-indazol-5-yl)- 1,2,4-oxadiazol-3- yl)phenol | 320.35 | 321.1 |
| 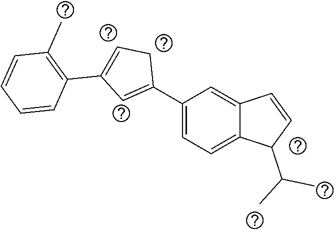 [SMILES: Cc1ccccc1C1=CCC(c2ccc3c(c2)C=CC3C(C)C)=C1] | 334 | 3-(2-isopropoxy- phenyl)-5-(1- isopropyl-1H-indazol- 5-yl)-1,2,4-oxadiazole | 362.43 | 363.1 |
| 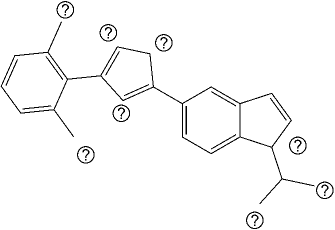 [SMILES: Cc1cccc(C)c1C1=CCC(c2ccc3c(c2)C=CC3C(C)C)=C1] | 335 | 3-(2,6-dimethoxy- phenyl)-5-(1- isopropylindazol- 5-yl)-1,2,4-oxadiazole | 364.41 | 365.1 |
| 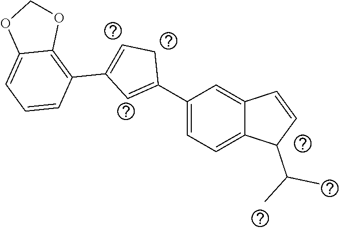 [SMILES: CC(C)C1C=Cc2cc(C3=CC(c4cccc5c4OCO5)=CC3)ccc21] | 336 | 3-(benzo[d][1,3]dioxol- 4-yl)-5-(1-isopropyl- 1H-indazol-5-yl)-1,2,4- oxadiazole | 348.36 | 349.1 |
| 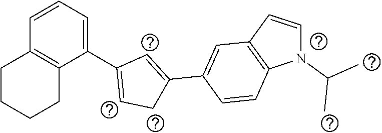 [SMILES: CC(C)n1ccc2cc(C3=CC(c4cccc5c4CCCC5)=CC3)ccc21] | 337 | 3-chroman-8-yl-5-(1- isopropylindazol-5-yl)- 1,2,4-oxadiazole | 360.42 | 361.1 |
| 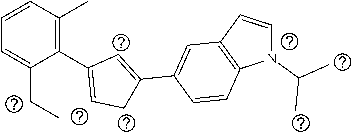 [SMILES: CCc1cccc(C)c1C1=CCC(c2ccc3c(ccn3C(C)C)c2)=C1] | 338 | 3-(2-fluoro-6-methoxy- phenyl)-5-(1-isopropyl- indazol-5-yl)-1,2,4- oxadiazole | 352.37 | 353.1 |
| 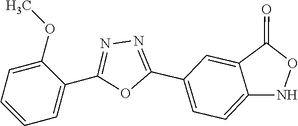 [SMILES: COc1ccccc1-c1nnc(-c2ccc3[nH]oc(=O)c3c2)o1] | 339 | 5-[5-(2-methoxyphenyl)- 1,3,4-oxadiazol-2- yl]-1,3-dihydro-2,1- benzoxazol-3-one | 309.28 | 310.2 |
| 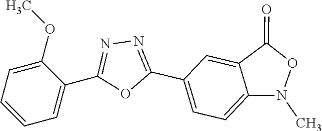 [SMILES: COc1ccccc1-c1nnc(-c2ccc3c(c2)c(=O)on3C)o1] | 340 | 5-[5-(2-methoxyphenyl)- 1,3,4-oxadiazol-2-yl]- 1-methyl-1,3-dihydro- 2,1-benzoxazol-3-one | 323.31 | 324.1 |
| 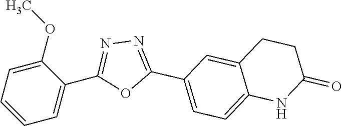 [SMILES: COc1ccccc1-c1nnc(-c2ccc3c(c2)CCC(=O)N3)o1] | 341 | 6-[5-(2-methoxyphenyl)- 1,3,4-oxadiazol-2- yl]-1,2,3,4-tetrahydro- quinolin-2-one | 321.34 | 322.1 |
| 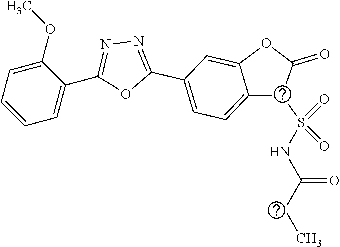 [SMILES: CCC(=O)NS(=O)(=O)C1C(=O)Oc2cc(-c3nnc(-c4ccccc4OC)o3)ccc21] | 342 | methyl N-({6-[5-(2- methoxyphenyl)-1,3,4- oxadiazol-2-yl]- 2-oxo-2,3-dihydro-1,3- benzoxazol-3- yl}sulfonyl)carbamate | 446.39 | 447 |
| 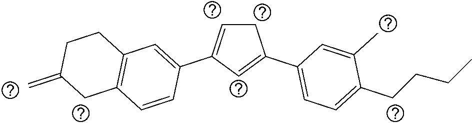 [SMILES: C=C1CCc2cc(C3=CCC(c4ccc(CCCC)c(C)c4)=C3)ccc2C1] | 343 | 2-[(2-fluoroethyl)amino]- 5-[3-(2-oxo-1,2,3,4- tetrahydroquinolin-6- yl)-1,2,4-oxadiazol-5- yl]benzonitrile | 377.38 | 378.1 |
| 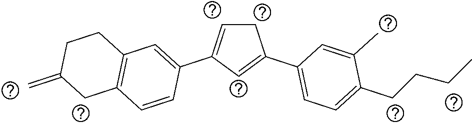 [SMILES: C=C1CCc2cc(C3=CCC(c4ccc(CCCC)c(C)c4)=C3)ccc2C1] | 344 | 2-[(2,2-difluoroethyl) amino]-5-[3-(2-oxo- 1,2,3,4-tetrahydroquinolin- 6-yl)-1,2,4-oxadiazol-5- yl]benzonitrile | 395.37 | 396.1 |
| 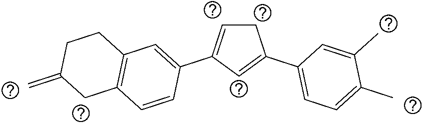 | 345 | 2-amino-5-[3-(2-oxo- 1,2,3,4-tetrahydro- quinolin-6-yl)-1,2,4- oxadiazol-5- yl]benzonitrile | 331.34 | 332.1 |
| 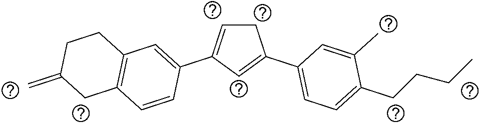 [SMILES: C=C1CCc2cc(C3=CCC(c4ccc(CCCC)c(C)c4)=C3)ccc2C1] | 346 | 2-[(2-fluoroprop-2-en- 1-yl)amino]-5-[3-(2- oxo-1,2,3,4-tetrahydro- quinolin-6-yl)-1,2,4- oxadiazol-5- yl]benzonilrile | 389.39 | 390.1 |
| 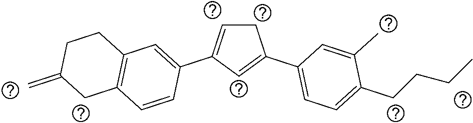 [SMILES: C=C1CCc2cc(C3=CCC(c4ccc(CCCC)c(C)c4)=C3)ccc2C1] | 347 | 2-[(2,2-difluoropropyl) amino]-5-[3-(2-oxo- 1,2,3,4-tetrahydro- quinolin-6-yl)-1,2,4- oxadiazol-5- yl]benzonitrile | 409.40 | 410.1 |
| 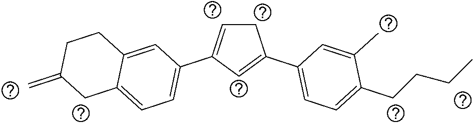 [SMILES: C=C1CCc2cc(C3=CCC(c4ccc(CCCC)c(C)c4)=C3)ccc2C1] | 348 | 2-[(2-fluoropropyl)amino]- 5-[3-(2-oxo-1,2,3,4- tetrahydroquinolin-6- yl)-1,2,4-oxadiazol-5- yl]benzonitrile | 391.41 | 392.1 |
| 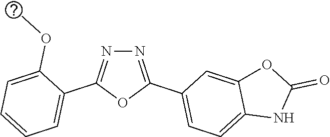 [SMILES: COc1ccccc1-c1nnc(-c2ccc3[nH]c(=O)oc3c2)o1] | 349 | 6-[5-(2-methoxyphenyl)- 1,3,4-oxadiazol-2- yl]-2,3-dihydro-1,3- benzoxazol-2-one | 309.28 | 310 |
| 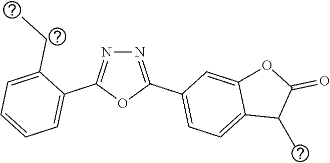 [SMILES: CCc1ccccc1-c1nnc(-c2ccc3c(c2)OC(=O)C3C)o1] | 350 | 6-[5-(2-methoxyphenyl)- 1,3,4-oxadiazol-2-yl]- 3-methyl-2,3-dihydro- 1,3-benzoxazol-2-one | 323.31 | 324.1 |
| 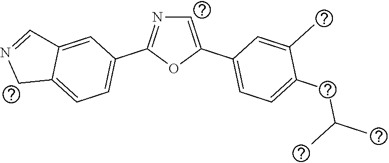 [SMILES: Cc1cc(-c2cnc(-c3ccc4c(c3)C=NC4)o2)ccc1CC(C)C] | 351 | 5-[5-(1H-indazol-5- yl)-1,3,4-oxadiazol- 2-yl]-2-[(propan-2- yl)amino]benzonitrile | 344.38 | 345.2 |
| 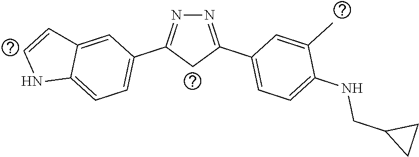 [SMILES: Cc1cc(C2=NN=C(c3ccc4[nH]ccc4c3)C2)ccc1NCC1CC1] | 352 | 2-[(cyclopropyl- methyl)aminol-5-[5- (1H-indazol-5-yl)-1,3,4- oxadiazol-2- yl]benzonitrile | 356.39 | 357.1 |
| 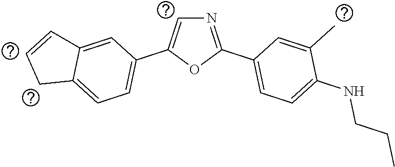 [SMILES: CCCNc1ccc(-c2ncc(-c3ccc4c(c3)C=CC4)o2)cc1C] | 353 | 2-[(2-fluoroethyl)amino]- 5-[5-(1H-indazol-5- yl)-1,3,4-oxadiazol-2- yl]benzonitrile | 348.34 | 349.1 |
| 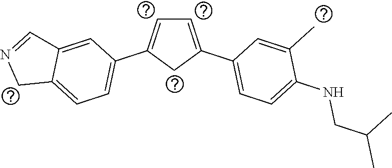 [SMILES: Cc1cc(C2=CC=C(c3ccc4c(c3)C=NC4)C2)ccc1NCC(C)C] | 354 | 2-[(2,2-difluoro- ethyl)amino]-5-[5-(1H- indazol-5-yl)-l,3,4- oxadiazol-2- yl]benzonitrile | 366.33 | 367.1 |
| 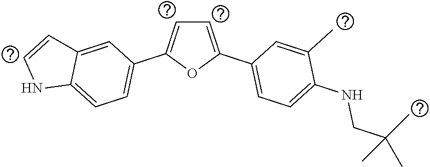 [SMILES: Cc1cc(-c2ccc(-c3ccc4[nH]ccc4c3)o2)ccc1NCC(C)(C)C] | 355 | 2-[(2,2-difluoro- propyl)amino]-5-[5- (1H-indazol-5-yl)- 1,3,4-oxadiazol- 2-yl]benzonitrile | 380.36 | 381.1 |
| 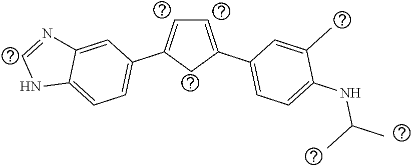 [SMILES: Cc1cc(C2=CC=C(c3ccc4[nH]cnc4c3)C2)ccc1NC(C)C] | 356 | 5-[5-(1H-1,3- benzodiazol-5-yl)- 1,3,4-oxadiazol-2- yl]-2-[(propan-2- yl)amino]benzonitrile | 344.38 | 345.1 |
| 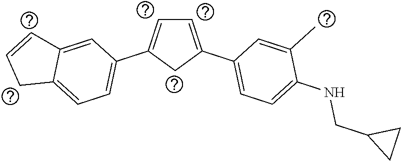 [SMILES: Cc1cc(C2=CC=C(c3ccc4c(c3)C=CC4)C2)ccc1NCC1CC1] | 357 | 5-[5-(1H-1,3-benzo- diazol-5-yl)-1,3,4- oxadiazol-2-yl]-2- [(cyclopropylmethyl) amino]benzonitrile | 356.39 | 357.1 |
| 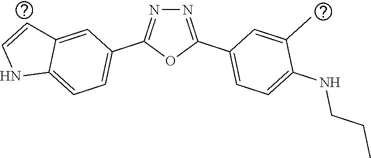 [SMILES: CCCNc1ccc(-c2nnc(-c3ccc4[nH]ccc4c3)o2)cc1C] | 358 | 5-[5-(1H-1,3-benzo- diazol-5-yl)-1,3,4- oxadiazol-2-yl]-2-[(2- fluoroethyl)amino] benzonitrile | 348.34 | 349.1 |
| 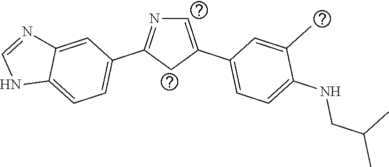 [SMILES: Cc1cc(C2=CN=C(c3ccc4[nH]cnc4c3)C2)ccc1NCC(C)C] | 359 | 5-[5-(1H-1,3-benzo- diazol-5-yl)-1,3,4- oxadiazol-2-yl]-2-[(2,2- difluoroethyl)amino] benzonitrile | 366.33 | 367.1 |
| 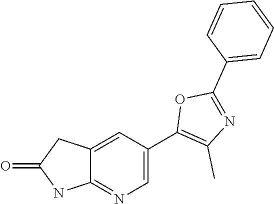 [SMILES: Cc1nc(-c2ccccc2)oc1-c1cnc2c(c1)CC(=O)N2] | 360 | 5-(4-methyl-2-phenyl- 1,3-oxazol-5-yl)- 1H,2H,3H-pyrrolo[2,3- b]pyridin-2-one | 291.1 | 292.1 |
| 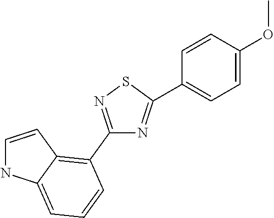 [SMILES: COc1ccc(-c2nc(-c3cccc4[nH]ccc34)ns2)cc1] | 361 | 4-[5-(4-methoxyphenyl)- 1,2,4-thiadiazol-3-yl]- 1H-indole | 307.08 | 308 |
| 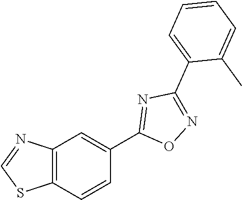 [SMILES: Cc1ccccc1-c1noc(-c2ccc3scnc3c2)n1] | 362 | 5-[3-(2-methylphenyl)- 1,2,4-oxadiazol-5-yl]- 1,3-benzothiazole | 293.06 | 294.2 |
| 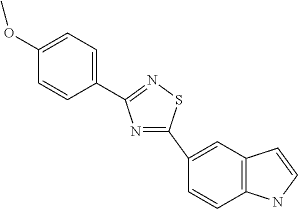 [SMILES: COc1ccc(-c2nsc(-c3ccc4[nH]ccc4c3)n2)cc1] | 363 | 5-[3-(4-methoxyphenyl)- 1,2,4-thiadiazol-5-yl]- 1H-indole | 307.08 | 308 |
| 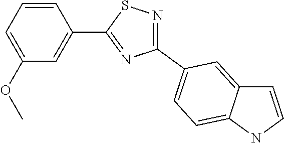 [SMILES: COc1cccc(-c2nc(-c3ccc4[nH]ccc4c3)ns2)c1] | 364 | 5-[5-(3-methoxyphenyl)- 1,2,4-thiadiazol-3-yl]- 1H-indole | 307.08 | 308 |
| 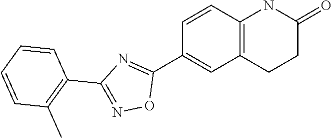 [SMILES: Cc1ccccc1-c1noc(-c2ccc3c(c2)CCC(=O)N3)n1] | 365 | 6-[3-(2-methylphenyl)-1,2,4- oxadiazol-5-yl]-1,2,3,4- tetrahydroquinolin-2-one | 305.12 | 306.1 |
| 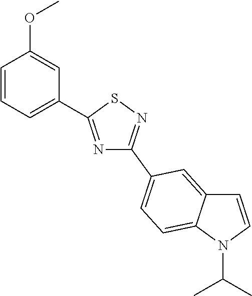 [SMILES: COc1cccc(-c2nc(-c3ccc4c(ccn4C(C)C)c3)ns2)c1] | 366 | 5-[5-(3-methoxyphenyl)- 1,2,4-thiadiazol- 3-yl]-1-(propan-2-yl)- 1H-indole | 349.12 | 350 |
| 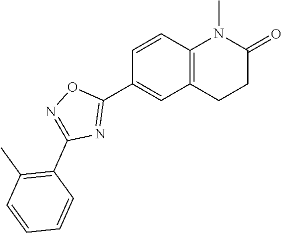 [SMILES: Cc1ccccc1-c1noc(-c2ccc3c(c2)CCC(=O)N3C)n1] | 367 | 1-methyl-6-[3-(2-methyl- phenyl)-1,2,4- oxadiazol-5-yl]-1,2,3,4- tetrahydroquinolin-2-one | 319.13 | 320.2 |
| 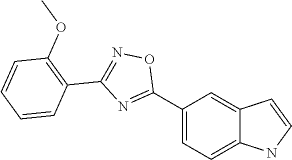 [SMILES: COc1ccccc1-c1noc(-c2ccc3[nH]ccc3c2)n1] | 368 | 5-(1H-indol-5-yl)-3- (2-methoxyphenyl)- 1,2,4-oxadiazole | 291.1 | 292 |
| 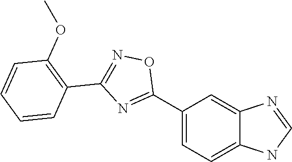 [SMILES: COc1ccccc1-c1noc(-c2ccc3[nH]cnc3c2)n1] | 369 | 5-(1H-benzo[d]imidazol- 5-yl)-3-(2-methoxy- phenyl)-1,2,4-oxadiazole | 292.1 | 293.2 |
| 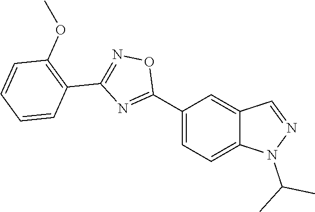 [SMILES: COc1ccccc1-c1noc(-c2ccc3c(cnn3C(C)C)c2)n1] | 370 | 5-(1-isopropyl-1H- indazol-5-yl)-3-(2- methoxyphenyl)-1,2,4- oxadiazole | 334.14 | 335.3 |
| 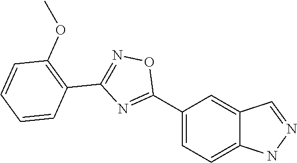 [SMILES: COc1ccccc1-c1noc(-c2ccc3[nH]ncc3c2)n1] | 371 | 5-(1H-indazol-5-yl)- 3-(2-methoxyphenyl)- 1,2,4-oxadiazole | 292.1 | 293.2 |
| 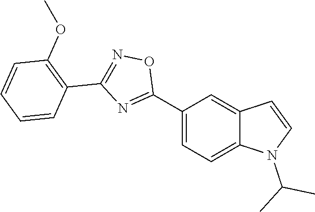 [SMILES: COc1ccccc1-c1noc(-c2ccc3c(ccn3C(C)C)c2)n1] | 372 | 5-(1-isopropyl-1H-indol- 5-yl)-3-(2-methoxy- phenyl)-1,2,4-oxadiazole | 333.15 | 334.1 |
| 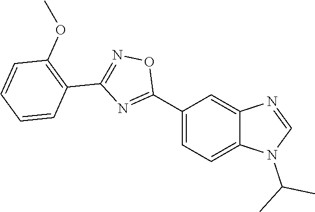 [SMILES: COc1ccccc1-c1noc(-c2ccc3c(c2)ncn3C(C)C)n1] | 373 | 5-(1-isopropyl-1H- benzo[d]imidazol-5- yl)-3-(2-methoxy- phenyl)-1,2,4-oxadiazole | 334.14 | 335.2 |
| 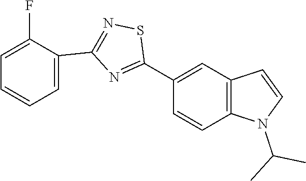 [SMILES: CC(C)n1ccc2cc(-c3nc(-c4ccccc4F)ns3)ccc21] | 374 | 5-[3-(2-fluorophenyl)- 1,2,4-thiadiazol-5- yl]-1-(propan-2-yl)- 1H-indole | 337.1 | 338 |
| 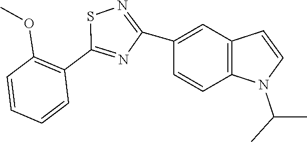 [SMILES: COc1ccccc1-c1nc(-c2ccc3c(ccn3C(C)C)c2)ns1] | 375 | 5-[5-(2-methoxy- phenyl)-1,2,4- thiadiazol-3-yl]-1- (propan-2-yl)- 1H-indole | 349.12 | 350 |
| 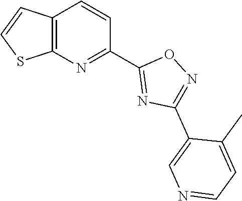 [SMILES: Cc1ccncc1-c1noc(-c2ccc3ccsc3n2)n1] | 376 | 4-methyl-3-(5- {thieno[2,3-b]pyridin- 6-yl}-1,2,4-oxadiazol-3- yl)pyridine | 294.06 | 295 |
| 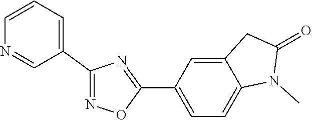 [SMILES: CN1C(=O)Cc2cc(-c3nc(-c4cccnc4)no3)ccc21] | 377 | 1-methyl-5-[3-(pyridin- 3-yl)-1,2,4-oxadiazol- 5-yl]-2,3-dihydro-1H- indol-2-one | 292.1 | 293.1 |
| 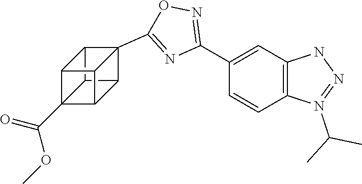 | 378 | 8-{3-[1-(propan-2-yl)- 1H-1,2,3-benzotriazol- 5-yl]-1,2,4-oxadiazol- 5-yl}cubane-1-carboxylate | 389.15 | 390 |
| 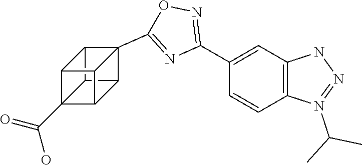 | 379 | 8-{3-[1-(propan-2-yl)- 1H-1,2,3-benzotriazol- 5-yl]-1,2,4-oxadiazol- 5-yl}cubane-1-carboxylic acid | 375.13 | 376 |
| 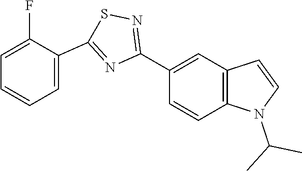 [SMILES: CC(C)n1ccc2cc(-c3nsc(-c4ccccc4F)n3)ccc21] | 380 | 5-[5-(2-fluorophenyl)- 1,2,4-thiadiazol-3- yl]-1-(propan-2-yl)- 1H-indole | 337.1 | 338 |
| 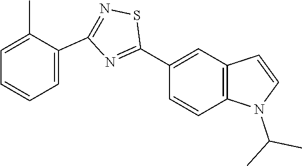 [SMILES: Cc1ccccc1-c1nsc(-c2ccc3c(ccn3C(C)C)c2)n1] | 381 | 5-[3-(2-methylphenyl)- 1,2,4-thiadiazol-5- yl]-1-(propan-2-yl)- 1H-indole | 333.13 | 334 |
| 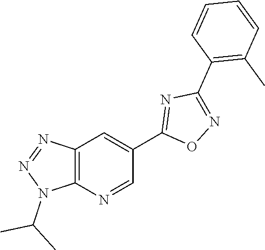 [SMILES: Cc1ccccc1-c1noc(-c2cnc3c(c2)nnn3C(C)C)n1] | 382 | 3-(2-methylphenyl)-5- [3-(propan-2-yl)-3H- [1,2,3]triazolo[4,5- b]pyridin-6-yl]- 1,2,4-oxadiazole | 320.14 | 321.2 |
| 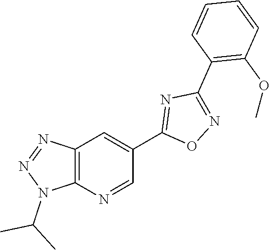 [SMILES: COc1ccccc1-c1noc(-c2cnc3c(c2)nnn3C(C)C)n1] | 383 | 3-(2-methoxyphenyl)- 5-[3-(propan-2-yl)- 3H-[1,2,3]triazolo[4,5- b]pyridin-6-yl]- 1,2,4-oxadiazole | 336.13 | 337.1 |
| 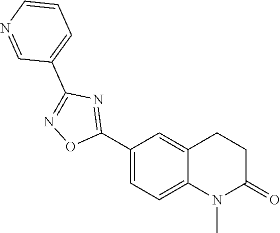 [SMILES: CN1C(=O)CCc2cc(-c3nc(-c4cccnc4)no3)ccc21] | 384 | 1-methyl-6-[3-(pyridin- 3-yl)-1,2,4-oxadiazol- 5-yl]-1,2,3,4- tetrahydroquinolin- 2-one | 306.11 | 307.1 |
| 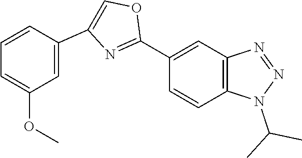 [SMILES: COc1cccc(-c2coc(-c3ccc4c(c3)nnn4C(C)C)n2)c1] | 385 | 5-[4-(3-methoxyphenyl)- 1,3-oxazol-2-yl]-1- (propan-2-yl)-H- 1,2,3-benzotriazole | 334.14 | 335 |
| 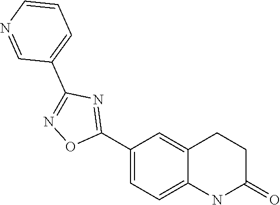 [SMILES: O=C1CCc2cc(-c3nc(-c4cccnc4)no3)ccc2N1] | 386 | 6-[3-(pyridin-3-yl)- 1,2,4-oxadiazol-5-yl]- 1,2,3,4- tetrahydro- quinolin-2-one | 292.1 | 293.1 |
| 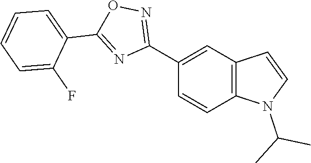 [SMILES: CC(C)n1ccc2cc(-c3noc(-c4ccccc4F)n3)ccc21] | 387 | 5-(2-fluorophenyl)- 3-(1-isopropyl-1H- indol-5-yl)-1,2,4- oxadiazole | 321.13 | 322.3 |
| 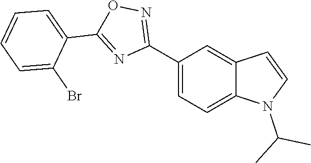 [SMILES: CC(C)n1ccc2cc(-c3noc(-c4ccccc4Br)n3)ccc21] | 388 | 5-(2-bromophenyl)- 3-(1-isopropyl-1H- indol-5-yl)-1,2,4- oxadiazole | 381.05 | 382.2 |
| 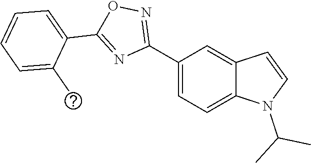 [SMILES: Cc1ccccc1-c1nc(-c2ccc3c(ccn3C(C)C)c2)no1] | 389 | 3-(1-isopropyl-1H- indol-5-yl)-5-(o- tolyl)-1,2,4-oxadiazole | 317.15 | 318.3 |
| 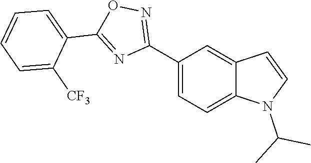 [SMILES: CC(C)n1ccc2cc(-c3noc(-c4ccccc4C(F)(F)F)n3)ccc21] | 390 | 3-(1-isopropyl-1H- indol-5-yl)-5-(2- (trifluoromethyl)phenyl)- 1,2,4-oxadiazole | 371.12 | 372.3 |
| 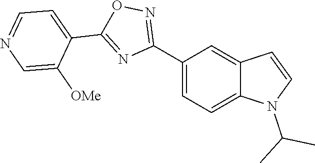 [SMILES: COc1cnccc1-c1nc(-c2ccc3c(ccn3C(C)C)c2)no1] | 391 | 3-(1-isopropyl-1H- indol-5-yl)-5-(3- methoxypyridin-4- yl)-1,2,4-oxadiazole | 334.14 | 335.3 |
| 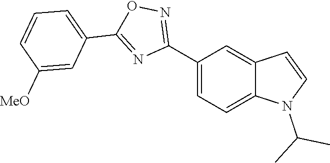 [SMILES: COc1cccc(-c2nc(-c3ccc4c(ccn4C(C)C)c3)no2)c1] | 392 | 3-(1-isopropyl-1H- indol-5-yl)-5-(3- methoxyphenyl)- 1,2,4-oxadiazole | 333.15 | 334.3 |
| 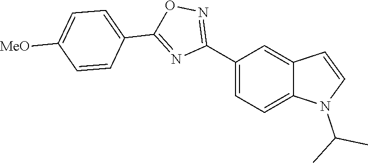 [SMILES: COc1ccc(-c2nc(-c3ccc4c(ccn4C(C)C)c3)no2)cc1] | 393 | 3-(1-isopropyl-1H- indol-5-yl)-5-(4- methoxyphenyl)- 1,2,4-oxadiazole | 333.15 | 334.2 |
| 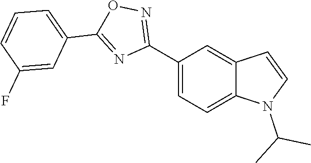 [SMILES: CC(C)n1ccc2cc(-c3noc(-c4cccc(F)c4)n3)ccc21] | 394 | 5-(3-fluorophenyl)- 3-(1-isopropyl- 1H-indol-5-yl)- 1,2,4-oxadiazole | 321.13 | 322.1 |
| 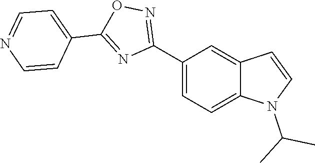 [SMILES: CC(C)n1ccc2cc(-c3noc(-c4ccncc4)n3)ccc21] | 395 | 3-(1-isopropyl-1H- indol-5-yl)-5- (pyridin-4-yl)- 1,2,4-oxadiazole | 304.13 | 305.1 |
| 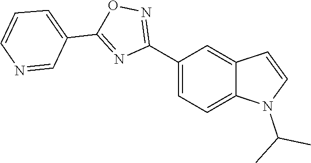 [SMILES: CC(C)n1ccc2cc(-c3noc(-c4cccnc4)n3)ccc21] | 396 | 3-(1-isopropyl-1H- indol-5-yl)-5- (pyridin-3-yl)- 1,2,4-oxadiazole | 304.13 | 305.1 |
| 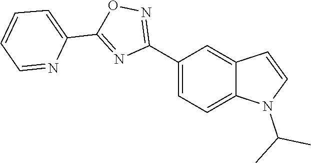 [SMILES: CC(C)n1ccc2cc(-c3noc(-c4ccccn4)n3)ccc21] | 397 | 3-(1-isopropyl-1H- indol-5-yl)-5- (pyridin-2-yl)- 1,2,4-oxadiazole | 304.13 | 305.1 |
| 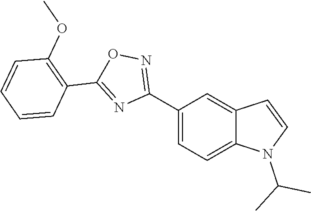 [SMILES: COc1ccccc1-c1nc(-c2ccc3c(ccn3C(C)C)c2)no1] | 398 | 3-(1-isopropyl-1H- indol-5-yl)-5-(2- methoxyphenyl)- 1,2,4-oxadiazole | 333.15 | 334.2 |
| 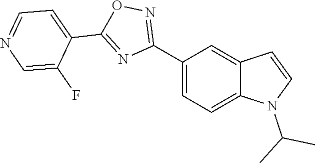 [SMILES: CC(C)n1ccc2cc(-c3noc(-c4ccncc4F)n3)ccc21] | 399 | 5-(3-fluoropyridin-4- yl)-3-(1-isopropyl-1H- indol-5-yl)-1,2,4- oxadiazole | 322.12 | 323 |
| 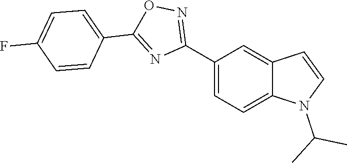 [SMILES: CC(C)n1ccc2cc(-c3noc(-c4ccc(F)cc4)n3)ccc21] | 400 | 5-(4-fluorophenyl)- 3-(1-isopropyl- 1H-indol-5-yl)- 1,2,4-oxadiazole | 321.13 | 322.1 |
| 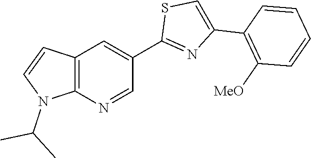 [SMILES: COc1ccccc1-c1csc(-c2cnc3c(ccn3C(C)C)c2)n1] | 401 | 2-(1-isopropyl-1H- pyrrolo[2,3-b]pyridin- 5-yl)-4-(2-methoxy- phenyl)thiazole | 349.12 | 350 |
| 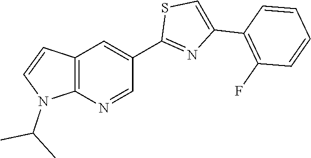 [SMILES: CC(C)n1ccc2cc(-c3nc(-c4ccccc4F)cs3)cnc21] | 402 | 4-(2-fluorophenyl)- 2-(1-isopropyl-1H- pyrrolo[2,3-b]pyridin- 5-yl)thiazole | 337.1 | 338.1 |
| 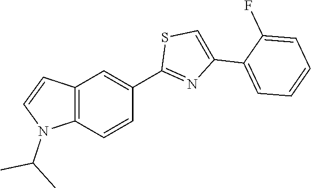 [SMILES: CC(C)n1ccc2cc(-c3nc(-c4ccccc4F)cs3)ccc21] | 403 | 4-(2-fluorophenyl)- 2-(1-isopropylindol- 5-yl)thiazole | 336.11 | 337.1 |
| 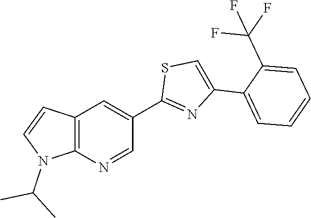 [SMILES: CC(C)n1ccc2cc(-c3nc(-c4ccccc4C(F)(F)F)cs3)cnc21] | 404 | 2-(1-isopropylindol- 5-yl)-4-[2-(trifluoro- methyl)phenyl]thiazole | 386.11 | 387.1 |
| 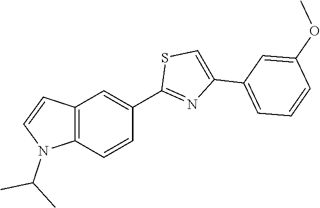 [SMILES: COc1cccc(-c2csc(-c3ccc4c(ccn4C(C)C)c3)n2)c1] | 405 | 2-(1-isopropylindol- 5-yl)-4-(3-methoxy- phenyl)thiazole | 348.13 | 349.1 |
| 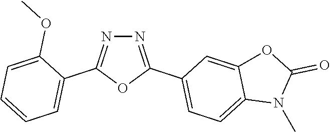 [SMILES: COc1ccccc1-c1nnc(-c2ccc3c(c2)oc(=O)n3C)o1] | 406 | 6-[5-(2-methoxy- phenyl)-1,3,4-oxadiazol- 2-yl]-3-methyl-2,3- dihydro-1,3- benzoxazol-2-one | 323.09 | 324.1 |
| 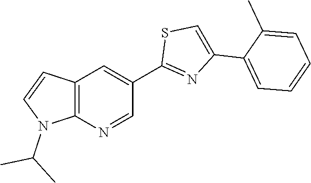 [SMILES: Cc1ccccc1-c1csc(-c2cnc3c(ccn3C(C)C)c2)n1] | 407 | 2-(1-isopropyl- pyrrolo[2,3-b]pyridin- 5-yl)-4-(o-tolyl)thiazole | 333.13 | 334.1 |
| 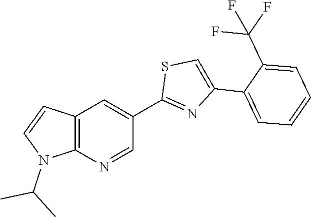 [SMILES: CC(C)n1ccc2cc(-c3nc(-c4ccccc4C(F)(F)F)cs3)cnc21] | 408 | 2-(1-isopropylpyrrolo[2,3- b]pyridin-5-yl)-4-[2- (trifluoromethyl)phenyl] thiazole | 387.1 | 388.1 |
| 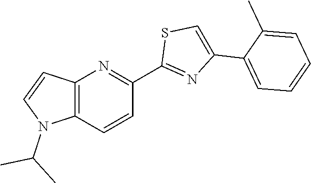 [SMILES: Cc1ccccc1-c1csc(-c2ccc3c(ccn3C(C)C)n2)n1] | 409 | 2-(1-isopropyl- pyrrolo [3,2-b]pyridin- 5-yl)-4-(o-tolyl)thiazole | 333.13 | 334.1 |
| 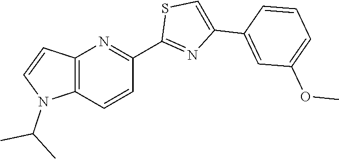 [SMILES: COc1cccc(-c2csc(-c3ccc4c(ccn4C(C)C)n3)n2)c1] | 410 | 2-(1-isoproplpyrrolo[3,2- b]pyridin-5-yl)-4-(3- methoxyphenyl)thiazole | 349.12 | 350.1 |
| 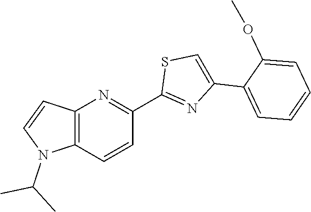 [SMILES: COc1ccccc1-c1csc(-c2ccc3c(ccn3C(C)C)n2)n1] | 411 | 2-(1-isopropyl-1H- pyrrolo[3,2- b]pyridin-5-yl)-4-(2- methoxyphenyl)thiazole | 349.12 | 350.1 |
| 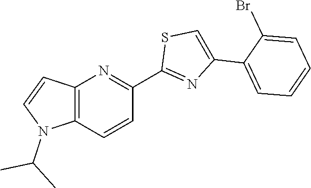 [SMILES: CC(C)n1ccc2nc(-c3nc(-c4ccccc4Br)cs3)ccc21] | 412 | 4-(2-bromophenyl)- 2-(1-isopropyl-1H- pyrrolo[3,2-b]pyridin- 5-yl)thiazole | 397.02 | 400 |
| 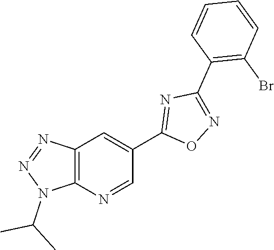 [SMILES: CC(C)n1nnc2cc(-c3nc(-c4ccccc4Br)no3)cnc21] | 413 | 3-(2-bromophenyl)- 5-[3-(propan-2-yl)- 3H-[1,2,3]triazolo[4,5- b]pyridin-6-yl]- 1,2,4-oxadiazole | 384.03 | 385.1 |
| 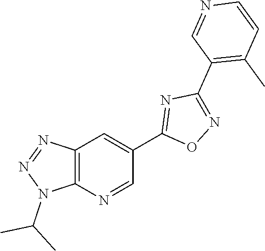 [SMILES: Cc1ccncc1-c1noc(-c2cnc3c(c2)nnn3C(C)C)n1] | 414 | 4-methyl-3-{5-[3- (propan-2-yl)- 3H-[1,2,3]triazolo[4,5- b]pyridin-6-yl]-1,2,4- oxadiazol-3-yl}pyridine | 321.13 | 322.1 |
| 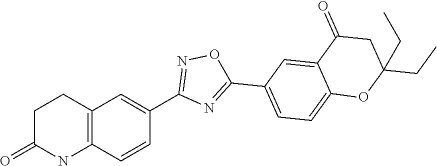 [SMILES: CCC1(CC)CC(=O)c2cc(-c3nc(-c4ccc5c(c4)CCC(=O)N5)no3)ccc2O1] | 415 | 6-(5-(2,2-diethyl-4- oxochroman-6-yl)- 1,2,4-oxadiazol-3-yl)- 3,4-dihydroquinolin- 2(1H)-one | 417.17 | 418.2 |
| 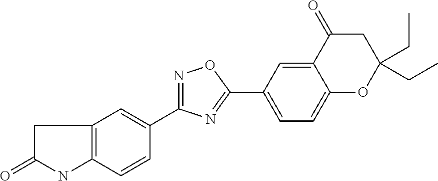 [SMILES: CCC1(CC)CC(=O)c2cc(-c3nc(-c4ccc5c(c4)CC(=O)N5)no3)ccc2O1] | 416 | 5-(5-(2,2-diethyl-4- oxochroman-6-yl)-1,2,4- oxadiazol-3-yl)indolin- 2-one | 403.15 | 404.1 |
| 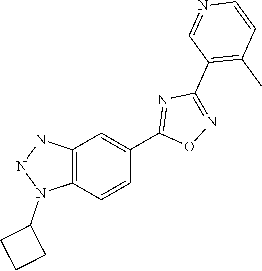 [SMILES: Cc1ccncc1-c1noc(-c2ccc3c(c2)NNN3C2CCC2)n1] | 417 | 1-cyclobutyl-5-[3-(4- methylpyridin-3-yl)-1,2,4- oxadiazol-5-yl]-1H-1,2,3- benzotriazole | 332.14 | 333.1 |
| 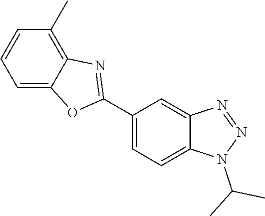 [SMILES: Cc1cccc2oc(-c3ccc4c(c3)nnn4C(C)C)nc12] | 418 | 2-(1-isopropyl-1H- benzo[d][1,2,3]triazol- 5-yl)-4-methyl- benzo[d]oxazole | 292.13 | 293.1 |
| 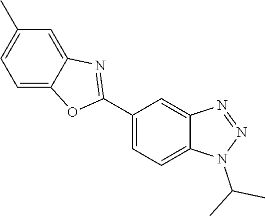 [SMILES: Cc1ccc2oc(-c3ccc4c(c3)nnn4C(C)C)nc2c1] | 419 | 2-(1-isopropyl-1H- benzo[d][1,2,3]triazol- 5-yl)-5-methyl- benzo[d]oxazole | 292.13 | 293.1 |
| 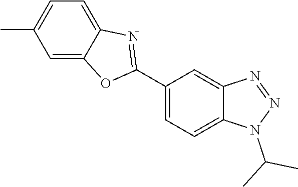 [SMILES: Cc1ccc2nc(-c3ccc4c(c3)nnn4C(C)C)oc2c1] | 420 | 2-(1-isopropyl-1H- benzo[d][1,2,3]triazol- 5-y l)-6-methyl- benzo[d]oxazole | 292.13 | 293.1 |
| 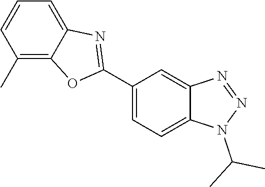 [SMILES: Cc1cccc2nc(-c3ccc4c(c3)nnn4C(C)C)oc12] | 421 | 2-(1-isopropyl-1H- benzo[d][1,2,3]triazol- 5-yl)-7-methyl- benzo[d]oxazole | 292.13 | 293.1 |
| 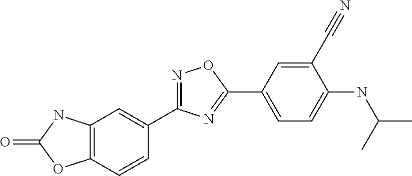 [SMILES: CC(C)Nc1ccc(-c2nc(-c3ccc4oc(=O)[nH]c4c3)no2)cc1C#N] | 422 | 2-(isopropylamino)- 5-(3-(2-oxo-2,3- dihydrobenzo[d]oxazol- 5-yl)-1,2,4-oxadiazol- 5-yl)benzonitrile | 361.12 | 362.1 |
| 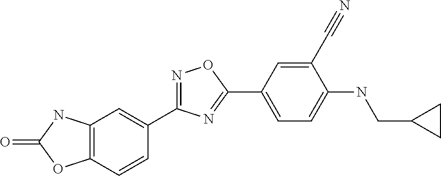 [SMILES: N#Cc1cc(-c2nc(-c3ccc4oc(=O)[nH]c4c3)no2)ccc1NCC1CC1] | 423 | 2-((cyclopropyl- methyl)amino)-5- (3-(2-oxo-2,3- dihydrobenzo[d]oxazol- 5-yl)-1,2,4-oxadiazol- 5-yl)benzonitrile | 373.12 | 374.2 |
| 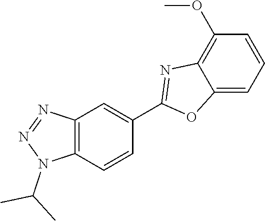 [SMILES: COc1cccc2oc(-c3ccc4c(c3)nnn4C(C)C)nc12] | 424 | 2-(1-isopropyl-1H- benzo[d][1,2,3]triazol- 5-yl)-4-methoxy- benzo[d]oxazole | 308.13 | 309 |
| 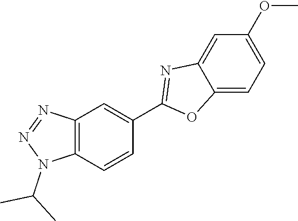 [SMILES: COc1ccc2oc(-c3ccc4c(c3)nnn4C(C)C)nc2c1] | 425 | 2-(1-isopropyl-1H- benzo[d][1,2,3]triazol- 5-yl)-5-methoxy- benzo[d]oxazole | 308.13 | 309.1 |
| 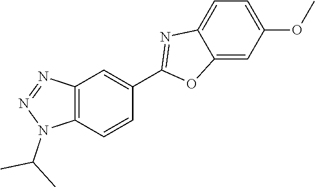 [SMILES: COc1ccc2nc(-c3ccc4c(c3)nnn4C(C)C)oc2c1] | 426 | 2-(1-isopropyl-1H- benzo[d][1,2,3]triazol- 5-yl)-6-methoxy- benzo[d]oxazole | 308.13 | 309 |
| 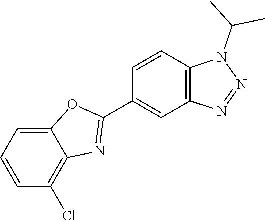 [SMILES: CC(C)n1nnc2cc(-c3nc4c(Cl)cccc4o3)ccc21] | 427 | 4-chloro-2-(1-isopropyl- 1H-benzo[d][1,2,3]triazol- 5-yl)benzo[d]oxazole | 312.08 | 313 |
| 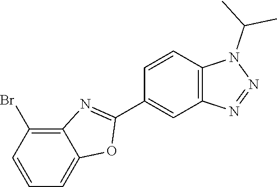 [SMILES: CC(C)n1nnc2cc(-c3nc4c(Br)cccc4o3)ccc21] | 428 | 4-bromo-2-(1- isopropylbenzotriazol- 5-yl)-1,3-benzoxazole | 356.03 | 357 |
| 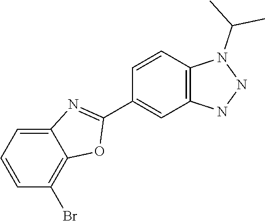 [SMILES: CC(C)N1NNc2cc(-c3nc4cccc(Br)c4o3)ccc21] | 429 | 7-bromo-2-(1-isopropyl- 1H-benzo[d][1,2,3]triazol- 5-yl)benzo[d]oxazole | 356.03 | 359 |
| 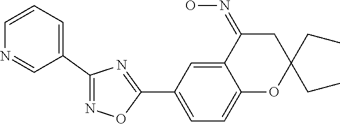 [SMILES: CCC1(CC)C/C(=N/O)c2cc(-c3nc(-c4cccnc4)no3)ccc2O1] | 430 | N-[(4Z)-2,2-diethyl-6- [3-(pyridin-3-yl)- 1,2,4-oxadiazol-5-yl]- 3,4-dihydro-2H-1- benzopyran-4- ylidene]hydroxylamine | 364.15 | 365.1 |
| 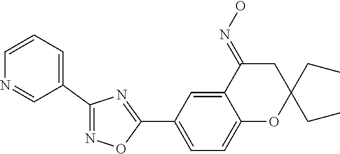 [SMILES: CCC1(CC)C/C(=N\O)c2cc(-c3nc(-c4cccnc4)no3)ccc2O1] | 431 | N-[(4E)-2,2-diethyl-6- [3-(pyridin-3-yl)- 1,2,4-oxadiazol-5-yl]- 3,4-dihydro-2H-1- benzopyran-4- ylidene]hydroxylamine | 364.15 | 365.1 |
| 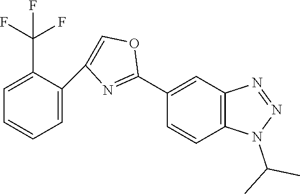 [SMILES: CC(C)n1nnc2cc(-c3nc(-c4ccccc4C(F)(F)F)co3)ccc21] | 432 | 1-(propan-2-yl)-5-{4-[2- (trifluoromethyl)phenyl]- 1,3-oxazol-2-yl}- 1H-1,2,3-benzotriazole | 372.12 | 373 |
| 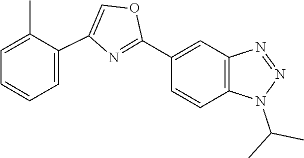 [SMILES: Cc1ccccc1-c1coc(-c2ccc3c(c2)nnn3C(C)C)n1] | 433 | 5-[4-(2-methylphenyl)- 1,3-oxazol-2-yl]-1- (propan-2-yl)-1H- 1,2,3-benzotriazole | 318.15 | 319 |
| 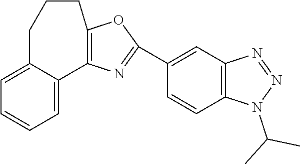 [SMILES: CC(C)n1nnc2cc(-c3nc4c(o3)CCCc3ccccc3-4)ccc21] | 434 | 4-[1-(propan-2-yl)-1H- 1,2,3-benzotriazol-5-yl]- 5-oxa-3- azatricyclo[8.4.0.0²,6] tetradeca- 1(14),2(6),3,10,12-pentaene | 344.16 | 345 |
| 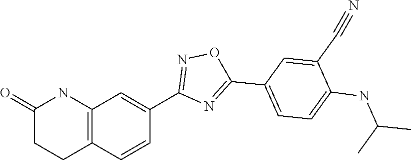 [SMILES: CC(C)Nc1ccc(-c2nc(-c3ccc4c(c3)NC(=O)CC4)no2)cc1C#N] | 435 | 2-(isopropylamino)- 5-(3-(2-oxo-1,2,3,4- tetrahydroquinolin- 7-yl)-1,2,4-oxadiazol- 5-yl)benzonitrile | 373.15 | 374 |
| 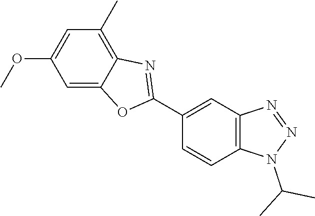 [SMILES: COc1cc(C)c2nc(-c3ccc4c(c3)nnn4C(C)C)oc2c1] | 436 | 2-(1-isopropyl-1H- benzo[d][1,2,3]triazol- 5-yl)-6-methoxy-4- methylbenzo[d]oxazole | 322.14 | 323 |
| 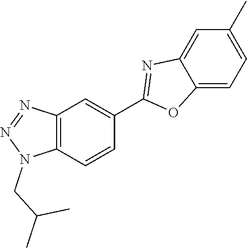 [SMILES: Cc1ccc2oc(-c3ccc4c(c3)nnn4CC(C)C)nc2c1] | 437 | 2-(1-isobutyl-1H- benzo[d][1,2,3]triazol- 5-yl)-5-methyl- benzo[d]oxazole | 306.15 | 307.2 |
| 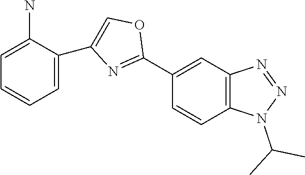 [SMILES: CC(C)n1nnc2cc(-c3nc(-c4ccccc4N)co3)ccc21] | 438 | 2-{2-[1-(propan-2-yl)- 1H-1,2,3-benzotriazol- 5-yl]-1,3-oxazol-4- yl}aniline | 319.14 | 320 |
| 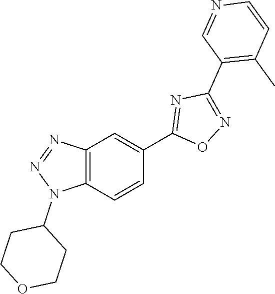 [SMILES: Cc1ccncc1-c1noc(-c2ccc3c(c2)nnn3C2CCOCC2)n1] | 439 | 5-[3-(4-methylpyridin- 3-yl)-1,2,4- oxadiazol-5-yl]-1- (oxan-4-yl)-1H- 1,2,3-benzotriazole | 362.15 | 363.1 |
| 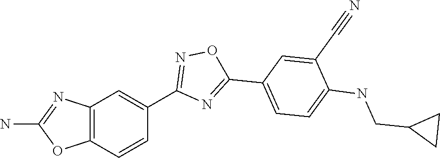 [SMILES: N#Cc1cc(-c2nc(-c3ccc4oc(N)nc4c3)no2)ccc1NCC1CC1] | 440 | 5-(3-(2-aminobenzo[d] thiazol-5-yl)-1,2,4- oxadiazol-5-yl)-2- ((cyclopropylmethyl) amino)benzonitrile | 388.11 | 389 |
| 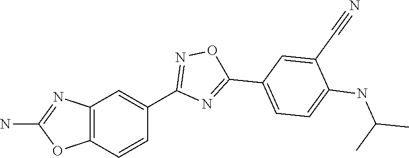 [SMILES: CC(C)Nc1ccc(-c2nc(-c3ccc4oc(N)nc4c3)no2)cc1C#N] | 441 | 5-(3-(2-aminobenzo[d] thiazol-5-yl)-1,2,4- oxadiazol-5-yl)-2- (isopropylamino) benzonitrile | 376.11 | 377 |
| 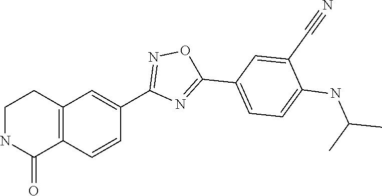 [SMILES: CC(C)Nc1ccc(-c2nc(-c3ccc4c(c3)CCNC4=O)no2)cc1C#N] | 442 | 2-(isopropylamino)-5- (3-(1-oxo-1,2,3,4- tetrahydroisoquinolin-6- yl)-1,2,4-oxadiazol- 5-yl)benzonitrile | 373.15 | 374 |
| 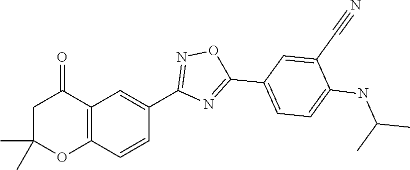 [SMILES: CC(C)Nc1ccc(-c2nc(-c3ccc4c(c3)C(=O)CC(C)(C)O4)no2)cc1C#N] | 443 | 5-(3-(2,2-diethyl-4- oxochroman-6- y)-1,2,4-oxadiazol- 5-yl)-2-(isopropyl- amino)benzonitrile | 430.2 | 431 |
| 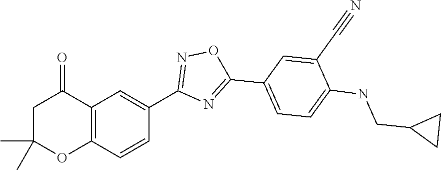 [SMILES: CC1(C)CC(=O)c2cc(-c3noc(-c4ccc(NCC5CC5)c(C#N)c4)n3)ccc2O1] | 444 | 2-((cyclopropyl- methyl)amino)-5- (3-(2,2-diethyl-4- oxochroman-6- yl)-1,2,4-oxadiazol-5- yl)benzonitrile | 442.2 | 443.3 |
| 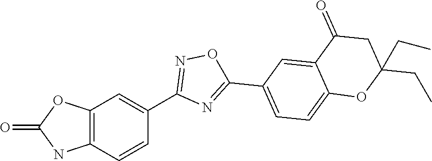 [SMILES: CCC1(CC)CC(=O)c2cc(-c3nc(-c4ccc5[nH]c(=O)oc5c4)no3)ccc2O1] | 445 | 6-(5-(2,2-diethyl-4- oxochroman-6- yl)-1,2,4-oxadiazol- 3-yl)benzo[d]oxazol- 2(3H)-one | 405.13 | 406 |
| 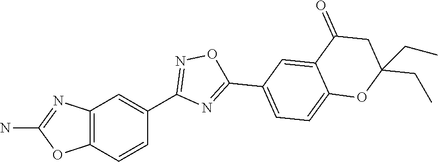 [SMILES: CCC1(CC)CC(=O)c2cc(-c3nc(-c4ccc5oc(N)nc5c4)no3)ccc2O1] | 446 | 6-(3-(2-aminobenzo [d]thiazol-5-yl)-1,2,4- oxadiazol-5-yl)-2,2- diethylchroman-4-one | 420.13 | 421 |
| 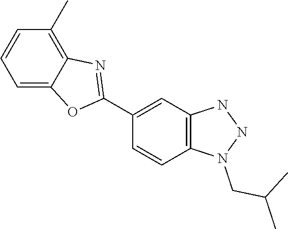 [SMILES: Cc1cccc2oc(-c3ccc4c(c3)NNN4CC(C)C)nc12] | 447 | 2-(1-isobutyl-1H- benzo[d][1,2,3]triazol- 5-yl)-4-methyl- benzo[d]oxazole | 306.15 | 307 |
| 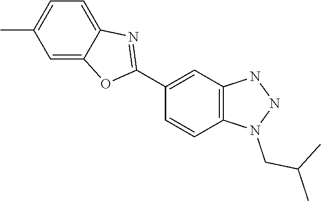 [SMILES: Cc1ccc2nc(-c3ccc4c(c3)NNN4CC(C)C)oc2c1] | 448 | 2-(1-isobutyl-1H- benzo[d][1,2,3]triazol- 5-yl)-6-methyl- benzo[d]oxazole | 306.15 | 307 |
| 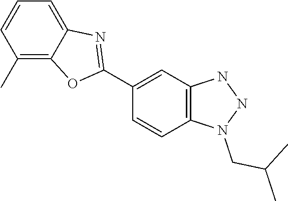 [SMILES: Cc1cccc2nc(-c3ccc4c(c3)NNN4CC(C)C)oc12] | 449 | 2-(1-isobutyl-1H- benzo[d][1,2,3]triazol- 5-yl)-7-methyl- benzo[d]oxazole | 306.15 | 307 |
| 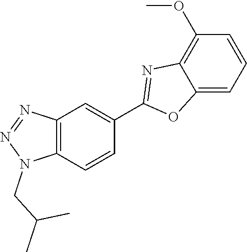 [SMILES: COc1cccc2oc(-c3ccc4c(c3)nnn4CC(C)C)nc12] | 450 | 2-(1-isobutyl-1H- benzo[d][1,2,3]triazol- 5-y l)-4-methoxy- benzo[d]oxazole | 322.14 | 323 |
| 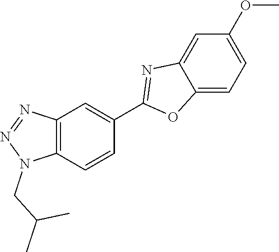 [SMILES: COc1ccc2oc(-c3ccc4c(c3)nnn4CC(C)C)nc2c1] | 451 | 2-(1-isobutyl-1H- benzo[d][1,2,3]triazol- 5-yl)-5-methoxy- benzo[d]oxazole | 322.14 | 323 |
| 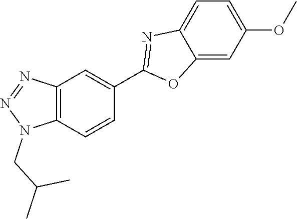 [SMILES: COc1ccc2nc(-c3ccc4c(c3)nnn4CC(C)C)oc2c1] | 452 | 2-(1-isobutyl-1H- benzo[d][1,2,3]triazol- 5-yl)-6-methoxy- benzo[d]oxazole | 322.14 | 323 |
| 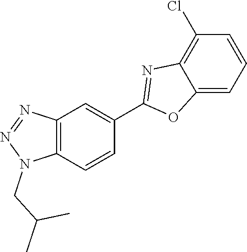 [SMILES: CC(C)Cn1nnc2cc(-c3nc4c(Cl)cccc4o3)ccc21] | 453 | 4-chloro-2-(1-isobutyl-1H- benzo[d][1,2,3]triazol-5- yl)benzo[d]oxazole | 326.09 | 326.9 |
| 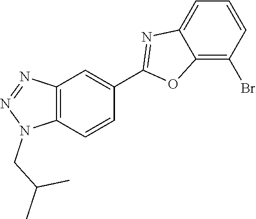 [SMILES: CC(C)Cn1nnc2cc(-c3nc4cccc(Br)c4o3)ccc21] | 454 | 7-bromo-2-(1-isobutyl-1H- benzo[d][1,2,3]triazol-5- yl)benzo[d]oxazole | 370.04 | 371.1 |
| 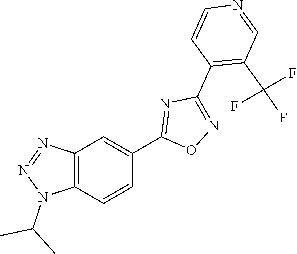 [SMILES: CC(C)n1nnc2cc(-c3nc(-c4ccncc4C(F)(F)F)no3)ccc21] | 455 | 1-(propan-2-yl)-5-{3-[3- (trifluoromethyl)pyridin- 4-yl]-1,2,4-oxadiazol- 5-yl}-1H-1,2,3- benzotriazole | 374.11 | 375.1 |
| 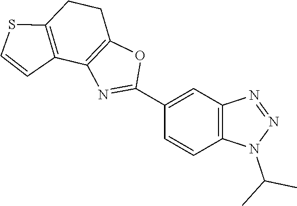 [SMILES: CC(C)n1nnc2cc(-c3nc4c(o3)CCc3sccc3-4)ccc21] | 456 | 4-[1-(propan-2-yl)- 1H-1,2,3-benzotriazol- 5-yl]-5-oxa-10-thia-3- azatricyclo[7.3.0.0²,6] dodeca-1(9),2(6),3,11- tetraene | 336.1 | 337 |
| 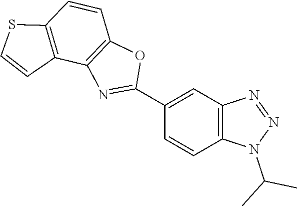 [SMILES: CC(C)n1nnc2cc(-c3nc4c(ccc5sccc54)o3)ccc21] | 457 | 4-[1-(propan-2-yl)- 1H-1,2,3-benzotriazol- 5-yl]-5-oxa-10-thia-3- azatricyclo[7.3.0.0²,6] dodeca-1(9),2(6),3,7,11- pentaene | 334.09 | 335 |
| 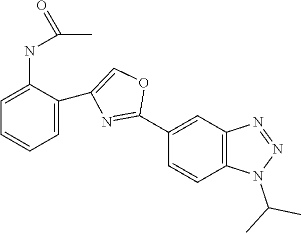 [SMILES: CC(=O)Nc1ccccc1-c1coc(-c2ccc3c(c2)nnn3C(C)C)n1] | 458 | N-(2-{2-[1-(propan-2-yl)- 1H-1,2,3-benzotriazol- 5-yl]-1,3-oxazol-4- yl}phenyl)acetamide | 361.15 | 362 |
| 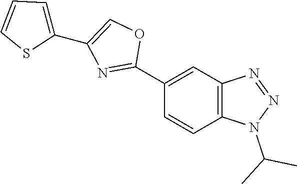 [SMILES: CC(C)n1nnc2cc(-c3nc(-c4cccs4)co3)ccc21] | 459 | 1-(propan-2-yl)-5-[4- (thiophen-2-yl)-1,3- oxazol-2-yl]-1H-1,2,3- benzotriazole | 310.09 | 311 |
| 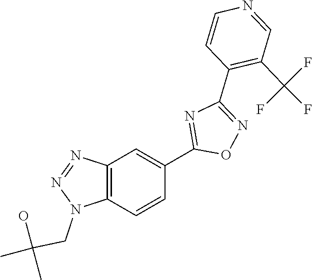 [SMILES: CC(C)(O)Cn1nnc2cc(-c3nc(-c4ccncc4C(F)(F)F)no3)ccc21] | 460 | 2-methyl-1-(5-{3-[3- (trifluoromethyl)pyridin- 4-yl]-1,2,4-oxadiazol- 5-yl}-1H-1,2,3- benzotriazol-1-yl)propan- 2-ol | 404.12 | 405.1 |
| 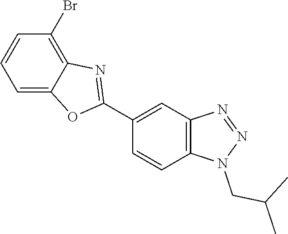 [SMILES: CC(C)Cn1nnc2cc(-c3nc4c(Br)cccc4o3)ccc21] | 461 | 4-bromo-2-(1-isobutyl-1H- benzo[d][1,2,3]triazol-5- yl)benzo[d]oxazole | 370.04 | 372.9 |
| 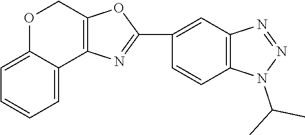 [SMILES: CC(C)n1nnc2cc(-c3nc4c(o3)COc3ccccc3-4)ccc21] | 462 | 5-{4H-chromeno[4,3- d][1,3]oxazol-2-yl}- 1-(propan-2-yl)- 1H-1,2,3-benzotriazole | 332.13 | 333 |
| 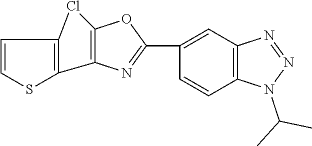 | 463 | 5-[4-(3-chlorothiophen- 2-yl)-1,3-oxazol-2- yl]-1-(propan-2-yl)-1H- 1,2,3-benzotriazole | 344.05 | 345 |
| 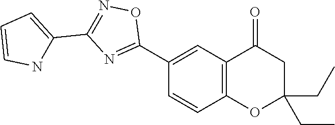 [SMILES: CCC1(CC)CC(=O)c2cc(-c3nc(-c4ccc[nH]4)no3)ccc2O1] | 464 | 6-(3-(1H-pyrrol-2-yl)- 1,2,4-oxadiazol-5-yl)- 2,2-diethylchroman- 4-one | 337.14 | 338.2 |
| 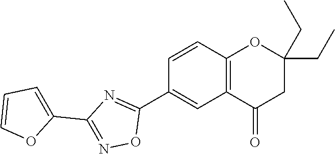 [SMILES: CCC1(CC)CC(=O)c2cc(-c3nc(-c4ccco4)no3)ccc2O1] | 465 | 2,2-diethyl-6-(3-(furan- 2-yl)-1,2,4-oxadiazol- 5-yl)chroman-4-one | 338.13 | 339.2 |
| 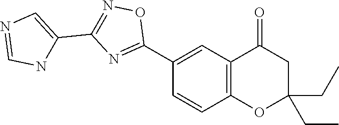 [SMILES: CCC1(CC)CC(=O)c2cc(-c3nc(-c4cnc[nH]4)no3)ccc2O1] | 466 | 6-(3-(1H-imidazol-5- yl)-1,2,4- oxadiazol-5-yl)-2,2- diethylchroman-4-one | 338.14 | 339.2 |
| 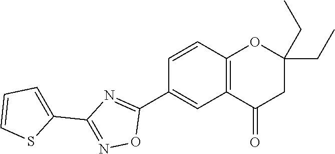 [SMILES: CCC1(CC)CC(=O)c2cc(-c3nc(-c4cccs4)no3)ccc2O1] | 467 | 2,2-diethyl-6-(3- (thiophen-2-yl)-1,2,4- oxadiazol-5-yl)chroman- 4-one | 354.1 | 355.2 |
| 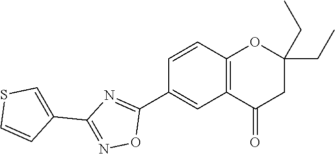 [SMILES: CCC1(CC)CC(=O)c2cc(-c3nc(-c4ccsc4)no3)ccc2O1] | 468 | 2,2-diethyl-6-(3- (thiophen-3-yl)-1,2,4- oxadiazol-5-yl)chroman- 4-one | 354.1 | 354.9 |
| 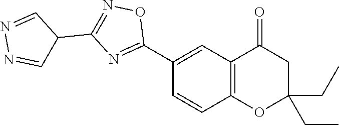 [SMILES: CCC1(CC)CC(=O)c2cc(-c3nc(C4C=NN=C4)no3)ccc2O1] | 469 | 6-(3-(1H-pyrazol-4-yl)- 1,2,4-oxadiazol-5- yl)-2,2-diethyl- chroman-4-one | 338.14 | 339.1 |
| 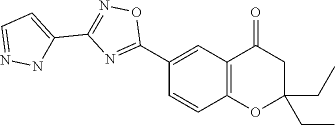 [SMILES: CCC1(CC)CC(=O)c2cc(-c3nc(-c4ccn[nH]4)no3)ccc2O1] | 470 | 6-(3-(1H-pyrazol-5-yl)- 1,2,4-oxadiazol-5- yl)-2,2-diethyl- chroman-4-one | 338.14 | 339.1 |
| 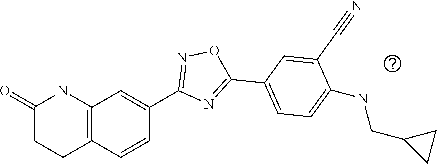 [SMILES: N#Cc1cc(-c2nc(-c3ccc4c(c3)NC(=O)CC4)no2)ccc1NCC1CC1] | 471 | 2-((cyclopropyl- methyl)amino)-5- (3-(2-oxo-1,2,3,4- tetrahydroquinolin- 7-yl)-1,2,4-oxadiazol- 5-yl)benzonitrile | | 385.43 |
| 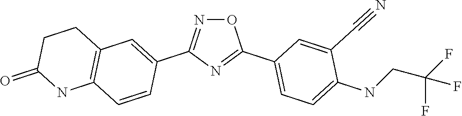 [SMILES: N#Cc1cc(-c2nc(-c3ccc4c(c3)CCC(=O)N4)no2)ccc1NCC(F)(F)F] | 472 | 5-[3-(2-oxo-1,2,3,4- tetrahydroquinolin-6- yl)-1,2,4-oxadiazol- 5-yl]-2-[(2,2,2-trifluoro- ethyl)amino]benzonitrile | 413.11 | 414.1 |
| 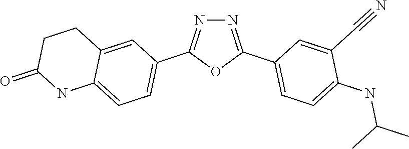 [SMILES: CC(C)Nc1ccc(-c2nnc(-c3ccc4c(c3)CCC(=O)N4)o2)cc1C#N] | 473 | 5-[5-(2-oxo-1,2,3,4- tetrahydroquinolin-6-yl)- 1,3,4-oxadiazol-2- yl]-2-[(propan-2- yl)amino]benzonitrile | 373.15 | 374.1 |
| 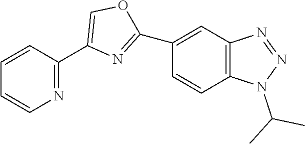 [SMILES: CC(C)n1nnc2cc(-c3nc(-c4ccccn4)co3)ccc21] | 474 | 1-(propan-2-yl)-5-[4- (pyridin-2-yl)-1,3- oxazol-2-yl]-1H-1,2,3- benzotriazole | 305.13 | 306 |
| 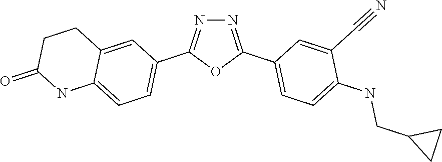 [SMILES: N#Cc1cc(-c2nnc(-c3ccc4c(c3)CCC(=O)N4)o2)ccc1NCC1CC1] | 475 | 2-[(cyclopropyl- methyl)amino]-5- [5-(2-oxo-1,2,3,4- tetrahydroquinolin-6-yl)- 1,3,4-oxadiazol-2- yl]benzonitrile | 385.15 | 386.1 |
| 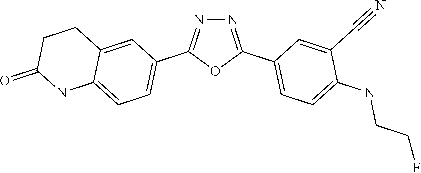 [SMILES: N#Cc1cc(-c2nnc(-c3ccc4c(c3)CCC(=O)N4)o2)ccc1NCCF] | 476 | 2-[(2-fluoroethyl)amino]- 5-[5-(2-oxo-1,2,3,4- tetrahydroquinolin-6- yl)-1,3,4-oxadiazol-2- yl]benzonitrile | 377.13 | 378.1 |
| 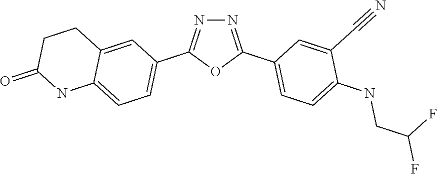 [SMILES: N#Cc1cc(-c2nnc(-c3ccc4c(c3)CCC(=O)N4)o2)ccc1NCC(F)F] | 477 | 2-[(2,2-difluoroethyl) amino]-5-[5-(2-oxo- 1,2,3,4-tetrahydro- quinolin-6-yl)-1,3,4- oxadiazol-2- yl]benzonitrile | 395.12 | 396.1 |
| 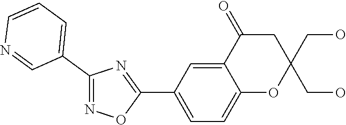 [SMILES: O=C1CC(CO)(CO)Oc2ccc(-c3nc(-c4cccnc4)no3)cc21] | 478 | 2,2-bis(hydroxymethyl)- 6-[3-(pyridin-3-yl)- 1,2,4-oxadiazol-5- yl]-3,4-dihydro-2H- 1-benzopyran-4-one | 353.1 | 354.1 |
| 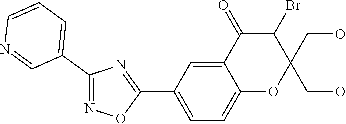 [SMILES: O=C1c2cc(-c3nc(-c4cccnc4)no3)ccc2OC(CO)(CO)C1Br] | 479 | 3 -bromo-2,2-bis(hydroxy- methyl)-6-[3-(pyridin-3- yl)-1,2,4-oxadiazol- 5-yl]-3,4-dihydro-2H-1- benzopyran-4-one | 431.01 | 432 |
| 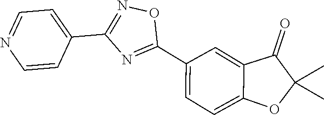 [SMILES: CC1(C)Oc2ccc(-c3nc(-c4ccncc4)no3)cc2C1=O] | 480 | 2,2-dimethyl-5-[3- (pyridin-4-yl)-1,2,4- oxadiazol-5-yl]-2,3- dihydro-1-benzofuran- 3-one | 307.1 | 308.1 |
| 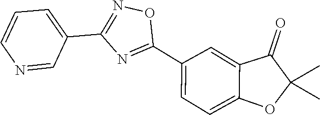 [SMILES: CC1(C)Oc2ccc(-c3nc(-c4cccnc4)no3)cc2C1=O] | 481 | 2,2-dimethyl-5-[3- (pyridin-3-yl)-1,2,4- oxadiazol-5-yl]-2,3- dihydro-1-benzofuran- 3-one | 307.1 | 308.1 |
| 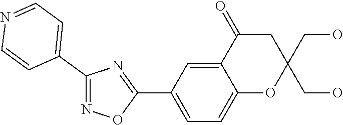 [SMILES: O=C1CC(CO)(CO)Oc2ccc(-c3nc(-c4ccncc4)no3)cc21] | 482 | 2,2-bis(hydroxymethyl)- 6-[3-(pyridin-4-yl)- 1,2,4-oxadiazol-5-yl]- 3,4-dihydro-2H-1- benzopyran-4-one | 353.1 | 354.1 |
| 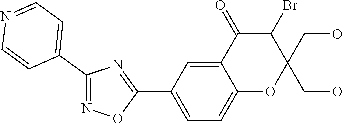 [SMILES: O=C1c2cc(-c3nc(-c4ccncc4)no3)ccc2OC(CO)(CO)C1Br] | 483 | 3-bromo-2,2-bis(hydroxy- methyl)-6-[3-(pyridin-4- yl)-1,2,4-oxadiazol- 5-yl]-3,4-dihydro-2H-1- benzopyran-4-one | 431.01 | 432 |
| 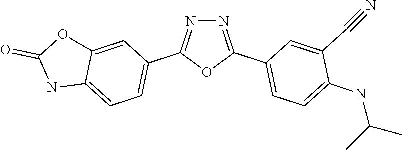 [SMILES: CC(C)Nc1ccc(-c2nnc(-c3ccc4[nH]c(=O)oc4c3)o2)cc1C#N] | 484 | 5-[5-(2-oxo-2,3-dihydro- 1,3-benzoxazol-6-yl)- 1,3,4-oxadiazol- 2-yl]-2-[(propan-2- yl)amino]benzonitrile | 361.12 | 362.1 |
| 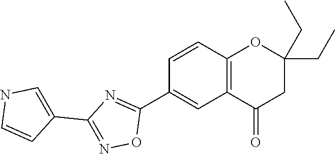 [SMILES: CCC1(CC)CC(=O)c2cc(-c3nc(-c4cc[nH]c4)no3)ccc2O1] | 485 | 6-(3-(1H-pyrrol-3-yl)- 1,2,4-oxadiazol-5-yl)- 2,2-diethylchroman- 4-one | 337.14 | 338 |
| 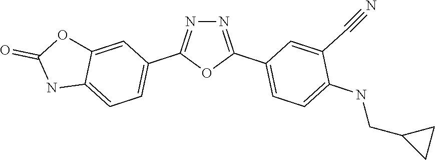 [SMILES: N#Cc1cc(-c2nnc(-c3ccc4[nH]c(=O)oc4c3)o2)ccc1NCC1CC1] | 486 | 2-[(cyclopropyl- methyl)amino]-5-[5- (2-oxo-2,3-dihydro-1,3- benzoxazol-6-yl)-1,3,4- oxadiazol-2- yl]benzonitrile | 373.12 | 374.1 |
| 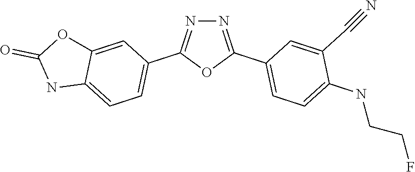 [SMILES: N#Cc1cc(-c2nnc(-c3ccc4[nH]c(=O)oc4c3)o2)ccc1NCCF] | 487 | 2-[(2-fluoroethyl)amino]- 5-[5-(2-oxo-2,3- dihydro-1,3-benzoxazol- 6-yl)-1,3,4-oxadiazol- 2-yl]benzonitrile | 365.09 | 366.1 |
| 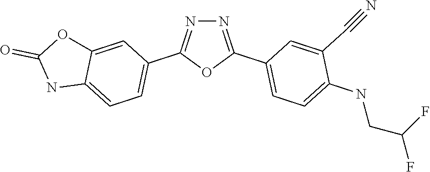 [SMILES: N#Cc1cc(-c2nnc(-c3ccc4[nH]c(=O)oc4c3)o2)ccc1NCC(F)F] | 488 | 2-[(2,2-difluoro- ethyl)amino]-5-[5- (2-oxo-2,3-dihydro-1,3- benzoxazol-6-yl)-1,3,4- oxadiazol-2- yl]benzonitrile | 383.08 | 384 |
| 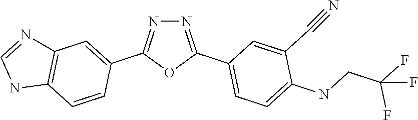 [SMILES: N#Cc1cc(-c2nnc(-c3ccc4[nH]cnc4c3)o2)ccc1NCC(F)(F)F] | 489 | 5-[3-(1H-1,3-benzo- diazol-5-yl)-1,2,4- oxadiazol-5-yl]-2- [(2,2,2-trifluoroethyl) amino]benzonitrile | 384.09 | 385.2 |
| 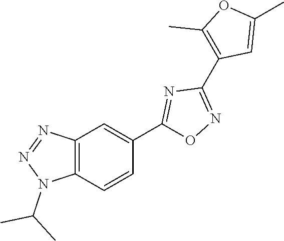 [SMILES: Cc1cc(-c2noc(-c3ccc4c(c3)nnn4C(C)C)n2)c(C)o1] | 490 | 5-[3-(2,5-dimethyl- furan-3-yl)-1,2,4- oxadiazol-5-yl]-1- (propan-2-yl)-1H- 1,2,3-benzotriazole | 323.14 | 324.1 |
| 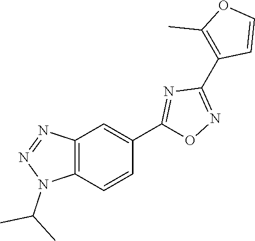 [SMILES: Cc1occc1-c1noc(-c2ccc3c(c2)nnn3C(C)C)n1] | 491 | 5-[3-(2-methylfuran-3- yl)-1,2,4-oxadiazol-5- yl]-1-(propan-2-yl)- 1H-1,2,3-benzotriazole | 309.12 | 310.1 |
| 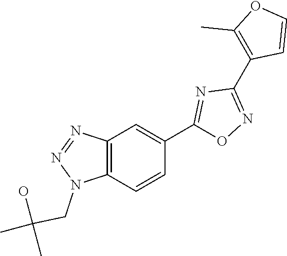 [SMILES: Cc1occc1-c1noc(-c2ccc3c(c2)nnn3CC(C)(C)O)n1] | 492 | 2-methyl-1-{5-[3-(2- methylfuran-3-yl)-1,2,4- oxadiazol-5-yl]-1H-1,2,3- benzotriazol-1-yl}propan- 2-ol | 339.13 | 340.1 |
| 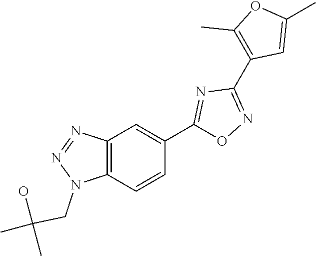 [SMILES: Cc1cc(-c2noc(-c3ccc4c(c3)nnn4CC(C)(C)O)n2)c(C)o1] | 493 | 1-{5-[3-(2,5-dimethyl- furan-3-yl)-1,2,4- oxadiazol-5-yl]-1H- 1,2,3-benzotriazol-1- yl}-2-methylpropan- 2-ol | 353.15 | 354.1 |
| 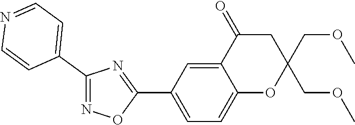 [SMILES: COCC1(COC)CC(=O)c2cc(-c3nc(-c4ccncc4)no3)ccc2O1] | 494 | 2,2-bis(methoxy- methyl)-6-[3-(pyridin-4- yl)-1,2,4-oxadiazol-5- yl]-3,4-dihydro-2H- 1-benzopyran-4-one | 381.13 | 382.1 |
| 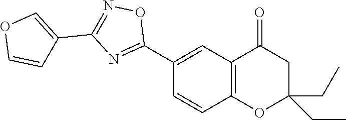 [SMILES: CCC1(CC)CC(=O)c2cc(-c3nc(-c4ccoc4)no3)ccc2O1] | 495 | 2,2-diethyl-6-(3-(furan- 3-yl)-1,2,4-oxadiazol- 5-yl)chroman-4-one | 338.13 | 339 |
| 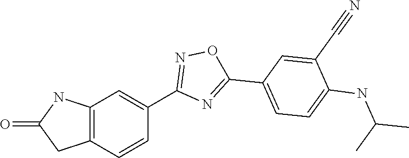 [SMILES: CC(C)Nc1ccc(-c2nc(-c3ccc4c(c3)NC(=O)C4)no2)cc1C#N] | 496 | 2-(isopropylamino)-5- (3-(2-oxoindolin-6- yl)-1,2,4-oxadiazol-5- yl)benzonitrile | 359.14 | 360.2 |
| 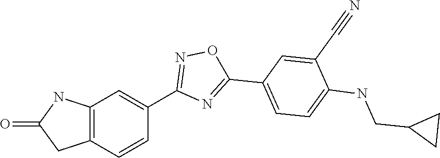 [SMILES: N#Cc1cc(-c2nc(-c3ccc4c(c3)NC(=O)C4)no2)ccc1NCC1CC1] | 497 | 2-((cyclopropyl- methyl)amino)-5-(3- (2-oxoindolin-6-yl)- 1,2,4-oxadiazol-5- yl)benzonitrile | 371.14 | 372.3 |
| 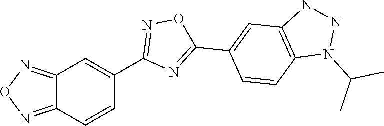 [SMILES: CC(C)N1NNc2cc(-c3nc(-c4ccc5nonc5c4)no3)ccc21] | 498 | 5-{5-[1-(propan-2-yl)- 1H-1,2,3-benzotriazol- 5-yl]-1,2,4-oxadiazol- 3-yl}-2,1,3- benzoxadiazole | 347.11 | 348.1 |
| 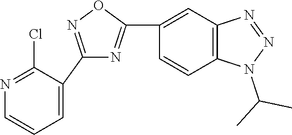 [SMILES: CC(C)n1nnc2cc(-c3nc(-c4cccnc4Cl)no3)ccc21] | 499 | 5-[3-(2-chloropyridin-3- yl)-1,2,4-oxadiazol- 5-yl]-1-(propan-2-yl)- 1H-1,2,3-benzotriazole | 340.08 | 341 |
| 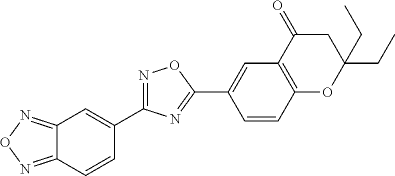 [SMILES: CCC1(CC)CC(=O)c2cc(-c3nc(-c4ccc5nonc5c4)no3)ccc2O1] | 500 | 6-[3-(2,1,3- benzoxadiazol-5- yl)-1,2,4-oxadiazol-5- yl]-2,2-diethyl-3,4- dihydro-2H-1- benzopyran-4-one | 390.13 | 391.1 |
| 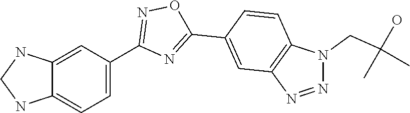 [SMILES: CC(C)(O)Cn1nnc2cc(-c3nc(-c4ccc5c(c4)NCN5)no3)ccc21] | 501 | 1-{5-[3-(1H-1,3-benzo- diazol-6-yl)-1,2,4- oxadiazol-5-yl]-1H- 1,2,3-benzotriazol-1- yl}-2-methylpropan- 2-ol | 375.14 | 376.1 |
| 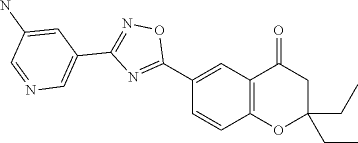 [SMILES: CCC1(CC)CC(=O)c2cc(-c3nc(-c4cncc(N)c4)no3)ccc2O1] | 502 | 6-(3-(5-aminopyridin- 3-yl)-1,2,4- oxadiazol-5-yl)-2,2- diethylchroman-4-one | 364.15 | 365.2 |
| 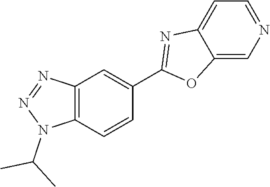 [SMILES: CC(C)n1nnc2cc(-c3nc4ccncc4o3)ccc21] | 503 | 2-(1-isopropyl-1H- benzo[d][1,2,3]triazol-5- yl)oxazolo[5,4-c]pyridine | 279.11 | 280.2 |
| 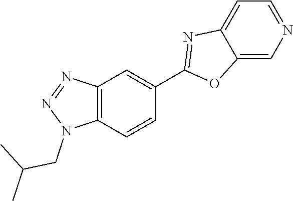 [SMILES: CC(C)Cn1nnc2cc(-c3nc4ccncc4o3)ccc21] | 504 | 2-(1-isobutyl-1H- benzo[d][1,2,3]triazol-5- yl)oxazolo[5,4-c]pyridine | 293.13 | 294.3 |
| 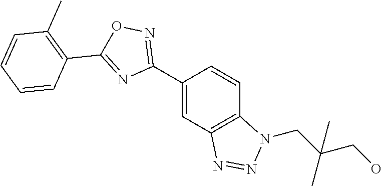 [SMILES: Cc1ccccc1-c1nc(-c2ccc3c(c2)nnn3CC(C)(C)CO)no1] | 505 | 2,2-dimethyl-3-(5- (5-(o-tolyl)-1,2,4- oxadiazol-3-yl)-1H- benzo[d][1,2,3]triazol- 1-yl)propan-1-ol | 363.17 | 364.1 |
| 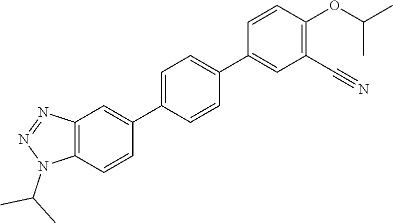 [SMILES: CC(C)Oc1ccc(-c2ccc(-c3ccc4c(c3)nnn4C(C)C)cc2)cc1C#N] | 506 | 4-isopropoxy-4′-(1- isopropyl-1H- benzo[d][1,2,3]triazol- 5-yl)-[1,1′-biphenyl]- 3-carbonitrile | 396.2 | 397.1 |
| 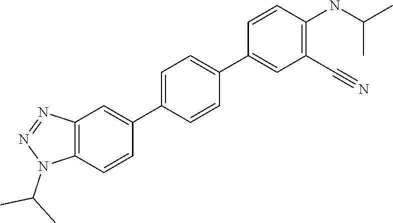 [SMILES: CC(C)Nc1ccc(-c2ccc(-c3ccc4c(c3)nnn4C(C)C)cc2)cc1C#N] | 507 | 4′-(1-isopropyl-1H- benzo[d][1,2,3]triazol- 5-yl)-4-(isopropylamino)- [1,1′-biphenyl]-3- carbonitrile | 395.21 | 396.1 |
| 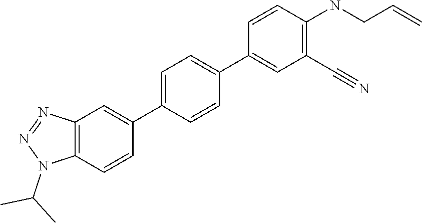 [SMILES: C=CCNc1ccc(-c2ccc(-c3ccc4c(c3)nnn4C(C)C)cc2)cc1C#N] | 508 | 4-(allylamino)-4′-(1- isopropyl-1H- benzo[d][1,2,3]triazol- 5-yl)-[1,1′-biphenyl]- 3-carbonitrile | 393.2 | 394.1 |
| 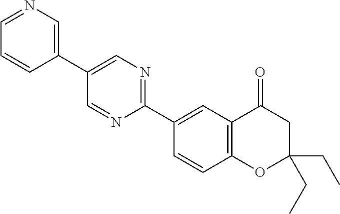 [SMILES: CCC1(CC)CC(=O)c2cc(-c3ncc(-c4cccnc4)cn3)ccc2O1] | 509 | 2,2-diethyl-6-(5-(pyridin- 3-yl)pyrimidin-2- yl)chroman-4-one | 359.16 | 360.3 |
| 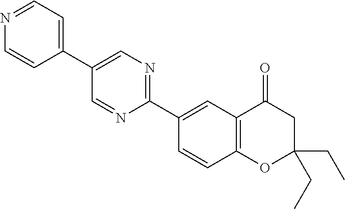 [SMILES: CCC1(CC)CC(=O)c2cc(-c3ncc(-c4ccncc4)cn3)ccc2O1] | 510 | 2,2-diethyl-6-(5-(pyridin- 4-yl)pyrimidin-2- yl)chroman-4-one | 359.16 | 360.3 |
| 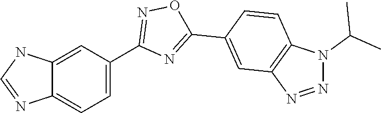 [SMILES: CC(C)n1nnc2cc(-c3nc(-c4ccc5nc[nH]c5c4)no3)ccc21] | 511 | 5-[3-(1H-1,3-benzodiazol- 6-yl)-1,2,4-oxadiazol- 5-yl]-1-(propan-2-yl)- 1H-1,2,3-benzotriazole | 345.13 | 346.1 |
| 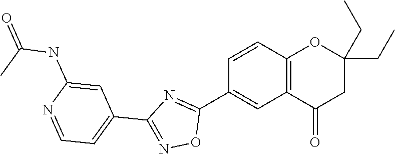 [SMILES: CCC1(CC)CC(=O)c2cc(-c3nc(-c4ccnc(NC(C)=O)c4)no3)ccc2O1] | 512 | N-(4-(5-(2,2-diethyl- 4-oxochroman-6-yl)- 1,2,4-oxadiazol-3- yl)pyridin-2-yl)acetamide | 406.16 | 407.3 |
| 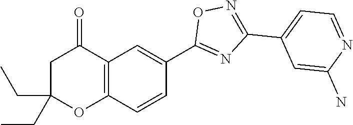 [SMILES: CCC1(CC)CC(=O)c2cc(-c3nc(-c4ccnc(N)c4)no3)ccc2O1] | 513 | 6-(3-(2-aminopyridin-4- yl)-1,2,4-oxadiazol- 5-yl)-2,2-diethyl- chroman-4-one | 364.15 | 365.4 |
| 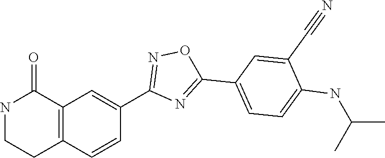 [SMILES: CC(C)Nc1ccc(-c2nc(-c3ccc4c(c3)C(=O)NCC4)no2)cc1C#N] | 514 | 2-(isopropylamino)-5- (3-(l-oxo-1,2,3,4-tetra- hydroisoquinolin-7-yl)- 1,2,4-oxadiazol-5- yl)benzonitrile | 373.15 | 374.3 |
| 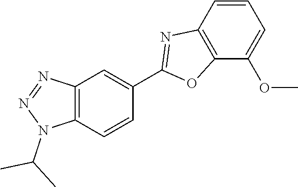 [SMILES: COc1cccc2nc(-c3ccc4c(c3)nnn4C(C)C)oc12] | 515 | 2-(1-isopropyl-1H- benzo[d][1,2,3]triazol- 5-yl)-7-methoxy- benzo[d]oxazole | 308.13 | 309.2 |
| 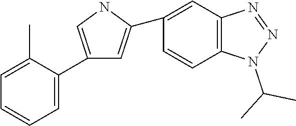 [SMILES: Cc1ccccc1-c1c[nH]c(-c2ccc3c(c2)nnn3C(C)C)c1] | 516 | 5-[4-(2-methylphenyl)- 1H-pyrrol-2-yl]-1- (propan-2-yl)-1H-1,2,3- benzotriazole | 316.17 | 317 |
| 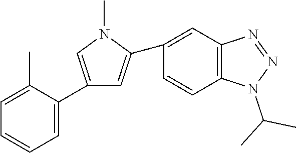 [SMILES: Cc1ccccc1-c1cc(-c2ccc3c(c2)nnn3C(C)C)n(C)c1] | 517 | 5-[1-methyl-4-(2- methylphenyl)- 1H-pyrrol-2-yl]-1- (propan-2-yl)-1H- 1,2,3-benzotriazole | 330.18 | 331 |
| 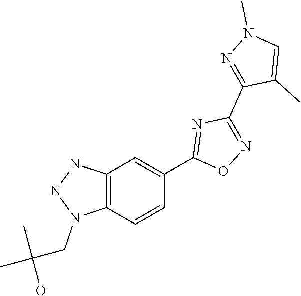 [SMILES: Cc1cn(C)nc1-c1noc(-c2ccc3c(c2)NNN3CC(C)(C)O)n1] | 518 | 1-{5-[3-(1,4-dimethyl- 1H-pyrazol-3-yl)- 1,2,4-oxadiazol-5-yl]-1H- 1,2,3-benzotriazol-1-yl}- 2-methylpropan-2-ol | 353.16 | 354.1 |
| 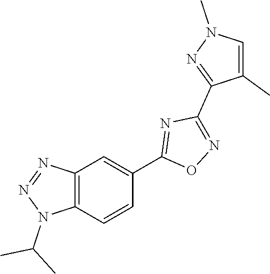 [SMILES: Cc1cn(C)nc1-c1noc(-c2ccc3c(c2)nnn3C(C)C)n1] | 519 | 5-[3-(1,4-dimethyl- 1H-pyrazol-3-yl)- 1,2,4-oxadiazol-5-yl]- 1-(propan-2-yl)-1H- 1,2,3-benzotriazole | 323.15 | 324.1 |
| 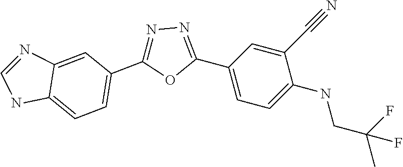 [SMILES: CC(F)(F)CNc1ccc(-c2nnc(-c3ccc4[nH]cnc4c3)o2)cc1C#N] | 520 | 5-[5-(1H-1,3-benzodiazol- 5-yl)-1,3,4-oxadiazol- 2-yl]-2-[(2,2-difluoro- propyl)amino]benzonitrile | 380.12 | 381.2 |
| 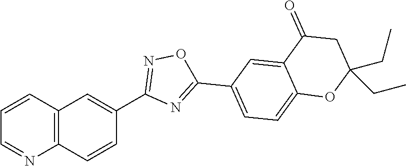 [SMILES: CCC1(CC)CC(=O)c2cc(-c3nc(-c4ccc5ncccc5c4)no3)ccc2O1] | 521 | 2,2-diethyl-6-(3-(quinolin- 6-yl)-1,2,4-oxadiazol- 5-yl)chroman-4-one | 399.16 | 400.4 |
| 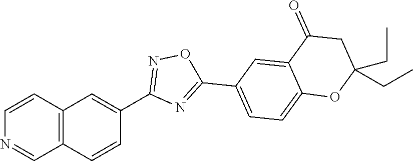 [SMILES: CCC1(CC)CC(=O)c2cc(-c3nc(-c4ccc5cnccc5c4)no3)ccc2O1] | 522 | 2,2-diethyl-6-(3- (isoquinolin-6-yl)- 1,2,4-oxadiazol-5- yl)chroman-4-one | 399.16 | 400.3 |
| 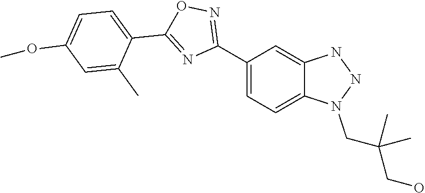 [SMILES: COc1ccc(-c2nc(-c3ccc4c(c3)NNN4CC(C)(C)CO)no2)c(C)c1] | 523 | 3-(5-(5-(4-methoxy-2- methylphenyl)-1,2,4- oxadiazol-3-yl)-1H- benzo[d][1,2,3]triazol- 1-yl)-2,2-dimethyl- propan-1-ol | 393.18 | 394.3 |
| 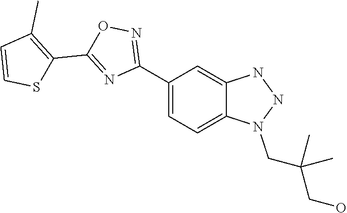 [SMILES: Cc1ccsc1-c1nc(-c2ccc3c(c2)NNN3CC(C)(C)CO)no1] | 524 | 2,2-dimethyl-3-(5-(5- (3-methylthiophen-2- yl)-1,2,4-oxadiazol- 3-yl)-1H-benzo[d] [1,2,3]triazol-1- yl)propan-1-ol | 369.13 | 370.2 |
| 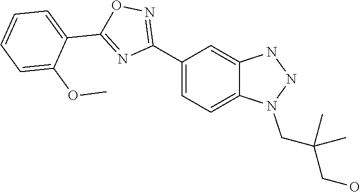 [SMILES: COc1ccccc1-c1nc(-c2ccc3c(c2)NNN3CC(C)(C)CO)no1] | 525 | 3-(5-(5-(2-methoxy- phenyl)-1,2,4- oxadiazol-3-yl)-1H- benzo[d][1,2,3]triazol- 1-yl)-2,2-dimethyl- propan-1-ol | 379.16 | 380 |
| 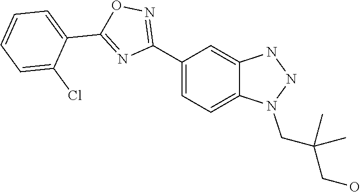 [SMILES: CC(C)(CO)CN1NNc2cc(-c3noc(-c4ccccc4Cl)n3)ccc21] | 526 | 3-(5-(5-(2-chloro- phenyl)-1,2,4- oxadiazol-3-yl)-1H- benzo[d][1,2,3]triazol- 1-yl)-2,2- dimethylpropan-1-ol | 383.11 | 384 |
| 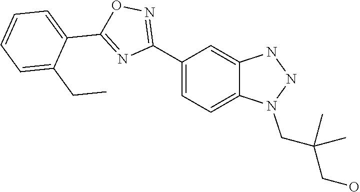 [SMILES: CCc1ccccc1-c1nc(-c2ccc3c(c2)NNN3CC(C)(C)CO)no1] | 527 | 3-(5-(5-(2-ethylphenyl)- 1,2,4-oxadiazol-3-yl)-1H- benzo[d][1,2,3]triazol- 1-yl)-2,2- dimethylpropan-1-ol | 377.19 | 378.1 |
| 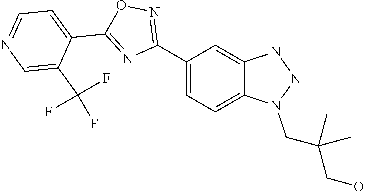 [SMILES: CC(C)(CO)CN1NNc2cc(-c3noc(-c4ccncc4C(F)(F)F)n3)ccc21] | 528 | 2,2-dimethyl-3-(5- (5-(3-(trifluoro- methyl)pyridin-4-yl)- 1,2,4-oxadiazol-3- yl)-1H-benzo[d][1,2,3] triazol-1-yl)propan- 1-ol | 418.14 | 419 |
| 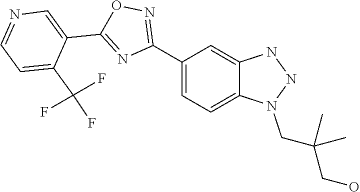 [SMILES: CC(C)(CO)CN1NNc2cc(-c3noc(-c4cnccc4C(F)(F)F)n3)ccc21] | 529 | 2,2-dimethyl-3-(5-(5- (4-(trifluoro- methyl)pyridin-3-yl)- 1,2,4-oxadiazol-3-yl)- 1H-benzo[d][1,2,3] triazol-1-yl)propan- 1-ol | 418.14 | 419 |
| 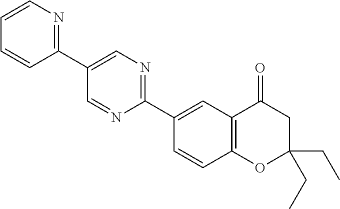 [SMILES: CCC1(CC)CC(=O)c2cc(-c3ncc(-c4ccccn4)cn3)ccc2O1] | 530 | 2,2-diethyl-6-(5- (pyridin-2-yl)pyrimidin- 2-yl)chroman-4-one | 359.16 | 360.3 |
| 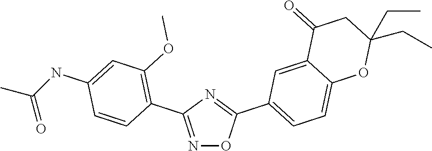 [SMILES: CCC1(CC)CC(=O)c2cc(-c3nc(-c4ccc(NC(C)=O)cc4OC)no3)ccc2O1] | 531 | N-{4-[5-(2,2-diethyl- 4-oxo-3,4-dihydro-2H- 1-benzopyran-6-yl)- 1,2,4-oxadiazol-3-yl]- 3-methoxyphenyl} acetamide | 435.18 | 436.2 |
| 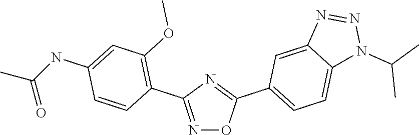 [SMILES: COc1cc(NC(C)=O)ccc1-c1noc(-c2ccc3c(c2)nnn3C(C)C)n1] | 532 | N-(3-methoxy-4-{5- [1-(propan-2-yl)-1H- 1,2,3-benzotriazol-5- yl]-1,2,4-oxadiazol-3- yl}phenyl)acetamide | 392.16 | 393.1 |
| 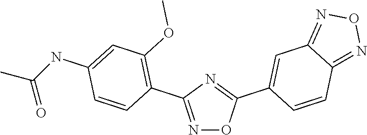 [SMILES: COc1cc(NC(C)=O)ccc1-c1noc(-c2ccc3nonc3c2)n1] | 533 | N-{4-[5-(2,1,3- benzoxadiazol-5- yl)-1,2,4-oxadiazol- 3-yl]-3-methoxy- phenyl}acetamide | 351.1 | 352 |
| 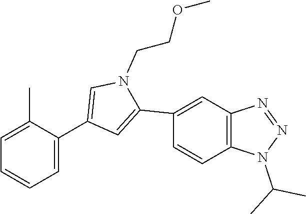 [SMILES: COCCn1cc(-c2ccccc2C)cc1-c1ccc2c(c1)nnn2C(C)C] | 534 | 5-[1-(2-methoxyethyl)- 4-(2-methylphenyl)- 1H-pyrrol-2-yl]-1- (propan-2-yl)-1H-1,2,3- benzotriazole | 374.21 | 375 |
| 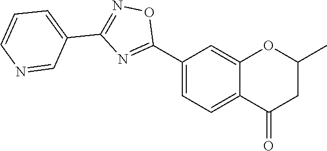 [SMILES: CC1CC(=O)c2ccc(-c3nc(-c4cccnc4)no3)cc2O1] | 535 | 2-methyl-7-[3-(pyridin- 3-yl)-1,2,4-oxadiazol- 5-yl]-3,4-dihydro-2H-1- benzopyran-4-one | 307.1 | 308 |
| 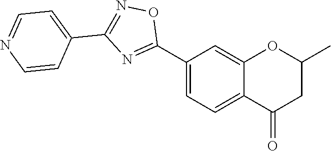 [SMILES: CC1CC(=O)c2ccc(-c3nc(-c4ccncc4)no3)cc2O1] | 536 | 2-methyl-7-[3-(pyridin- 4-yl)-1,2,4-oxadiazol- 5-yl]-3,4-dihydro-2H-1- benzopyran-4-one | 307.1 | 308.1 |
| 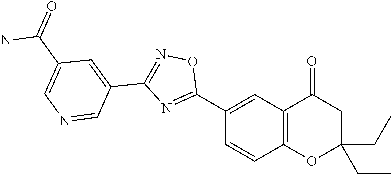 [SMILES: CCC1(CC)CC(=O)c2cc(-c3nc(-c4cncc(C(N)=O)c4)no3)ccc2O1] | 537 | 5-(5-(2,2-diethyl-4- oxochroman-6- yl)-1,2,4-oxadiazol-3- yl)nicotinamide | 392.15 | 393.2 |
| 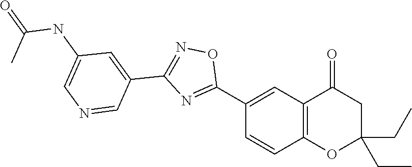 [SMILES: CCC1(CC)CC(=O)c2cc(-c3nc(-c4cncc(NC(C)=O)c4)no3)ccc2O1] | 538 | N-(5-(5-(2,2-diethyl-4- oxochroman-6-yl)- 1,2,4-oxadiazol- 3-yl)pyridin-3- yl)acetamide | 406.16 | 407.3 |
| 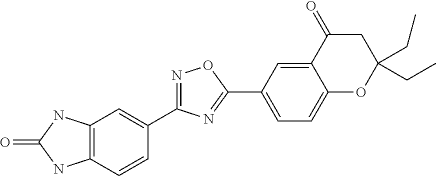 [SMILES: CCC1(CC)CC(=O)c2cc(-c3nc(-c4ccc5[nH]c(=O)[nH]c5c4)no3)ccc2O1] | 539 | 5-(5-(2,2-diethyl-4- oxochroman-6-yl)-1,2,4- oxadiazol-3-yl)-1H- benzo[d]imidazol- 2(3H)-one | 404.15 | 405 |
| 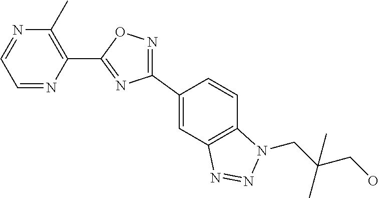 [SMILES: Cc1nccnc1-c1nc(-c2ccc3c(c2)nnn3CC(C)(C)CO)no1] | 540 | 2,2-dimethyl-3-(5-(5- (3-methylpyrazin-2- yl)-1,2,4-oxadiazol- 3-yl)-1H-benzo[d] [1,2,3]triazol-1- yl)propan-1-ol | 365.16 | 366.3 |
| 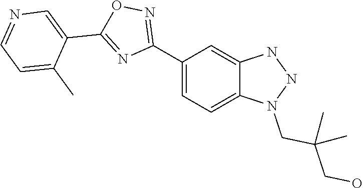 [SMILES: Cc1ccncc1-c1nc(-c2ccc3c(c2)NNN3CC(C)(C)CO)no1] | 541 | 2,2-dimethyl-3-(5-(5- (4-methylpyridin-3- yl)-1,2,4-oxadiazol- 3-yl)-1H-benzo[d] [1,2,3]triazol-1- yl)propan-1-ol | 364.16 | 365 |
| 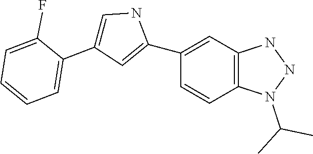 [SMILES: CC(C)N1NNc2cc(-c3cc(-c4ccccc4F)c[nH]3)ccc21] | 542 | 5-[4-(2-fluorophenyl)- 1H-pyrrol-2-yl]-1- (propan-2-yl)-1H-1,2,3- benzotriazole | 320.14 | 321.2 |
| 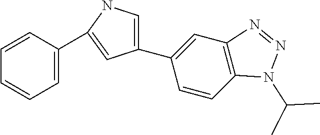 [SMILES: CC(C)n1nnc2cc(-c3c[nH]c(-c4ccccc4)c3)ccc21] | 543 | 5-(5-phenyl-1H-pyrrol-3- yl)-1-(propan-2-yl)-1H- 1,2,3-benzotriazole | 302.15 | 303 |
| 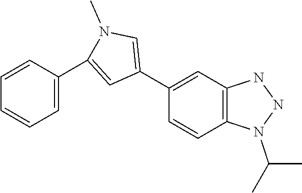 [SMILES: CC(C)N1NNc2cc(-c3cc(-c4ccccc4)n(C)c3)ccc21] | 544 | 5-(1-methyl-5-phenyl- 1H-pyrrol-3-yl)-1- (propan-2-yl)-1H-1,2,3- benzotriazole | 316.17 | 317 |
| 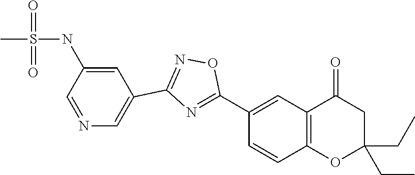 [SMILES: CCC1(CC)CC(=O)c2cc(-c3nc(-c4cncc(NS(C)(=O)=O)c4)no3)ccc2O1] | 545 | N-(5-(5-(2,2-diethyl- 4-oxochroman- 6-yl)-1,2,4-oxadiazol- 3-yl)pyridin-3- yl)methanesulfonamide | 442.13 | 443.3 |
| 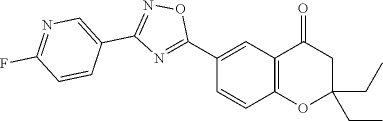 [SMILES: CCC1(CC)CC(=O)c2cc(-c3nc(-c4ccc(F)nc4)no3)ccc2O1] | 546 | 2,2-diethyl-6-(3-(6- fluoropyridin-3-yl)- 1,2,4-oxadiazol-5- yl)chroman-4-one | 367.13 | 368.3 |
| 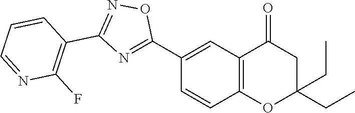 [SMILES: CCC1(CC)CC(=O)c2cc(-c3nc(-c4cccnc4F)no3)ccc2O1] | 547 | 2,2-diethyl-6-(3-(2- fluoropyridin-3- yl)-1,2,4-oxadiazol- 5-yl)chroman-4-one | 367.13 | 368.2 |
| 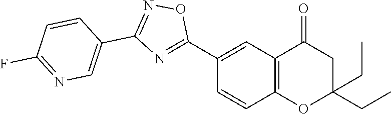 [SMILES: CCC1(CC)CC(=O)c2cc(-c3nc(-c4ccc(F)nc4)no3)ccc2O1] | 548 | 2,2-diethyl-6-(3-(6- (trifluoromethyl)pyridin- 3-yl)-1,2,4- oxadiazol-5-yl)chroman- 4-one | 417.13 | 418.2 |
| 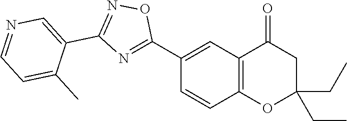 [SMILES: CCC1(CC)CC(=O)c2cc(-c3nc(-c4cnccc4C)no3)ccc2O1] | 549 | 2,2-diethyl-6-(3-(4- methylpyridin-3-yl)- 1,2,4-oxadiazol-5- yl)chroman-4-one | 363.16 | 364.3 |
| 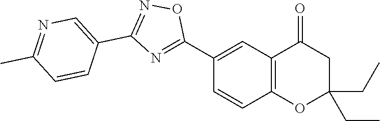 [SMILES: CCC1(CC)CC(=O)c2cc(-c3nc(-c4ccc(C)nc4)no3)ccc2O1] | 550 | 2,2-diethyl-6-(3-(6- methylpyridin-3-yl)- 1,2,4-oxadiazol-5- yl)chroman-4-one | 363.16 | 364.3 |
| 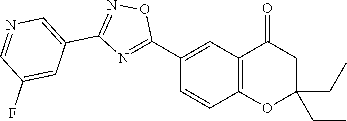 [SMILES: CCC1(CC)CC(=O)c2cc(-c3nc(-c4cncc(F)c4)no3)ccc2O1] | 551 | 2,2-diethyl-6-(3-(5- fluoropyridin-3-yl)- 1,2,4-oxadiazol-5- yl)chroman-4-one | 367.13 | 368.2 |
| 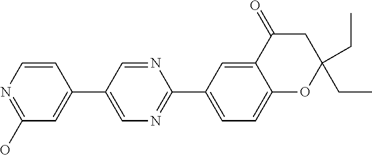 [SMILES: CCC1(CC)CC(=O)c2cc(-c3ncc(-c4ccnc(O)c4)cn3)ccc2O1] | 552 | 2,2-diethyl-6-(5-(2- hydroxypyridin-4- yl)pyrimidin-2- yl)chroman-4-one | 375.16 | 376.3 |
| 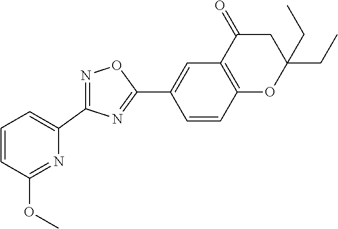 [SMILES: CCC1(CC)CC(=O)c2cc(-c3nc(-c4cccc(OC)n4)no3)ccc2O1] | 553 | 2,2-diethyl-6-[3-(6- methoxypyridin-2-yl)- 1,2,4-oxadiazol-5-yl]- 3,4-dihydro-2H-1- benzopyran-4-one | 379.15 | 380.1 |
| 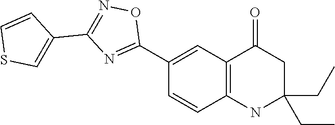 [SMILES: CCC1(CC)CC(=O)c2cc(-c3nc(-c4ccsc4)no3)ccc2N1] | 554 | 2,2-diethyl-6-(3- (thiophen-3-yl)-1,2,4- oxadiazol-5-yl)-2,3- dihydroquinolin-4(1H)- one | 353.12 | 354.2 |
| 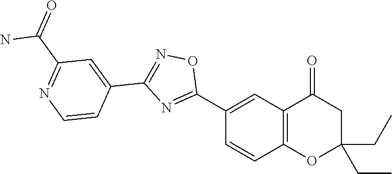 [SMILES: CCC1(CC)CC(=O)c2cc(-c3nc(-c4ccnc(C(N)=O)c4)no3)ccc2O1] | 555 | 4-(5-(2,2-diethyl-4- oxochroman-6-yl)- 1,2,4-oxadiazol-3- yl)picolinamide | 392.15 | 393 |
| 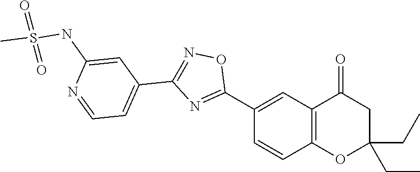 [SMILES: CCC1(CC)CC(=O)c2cc(-c3nc(-c4ccnc(NS(C)(=O)=O)c4)no3)ccc2O1] | 556 | N-(4-(5-(2,2-diethyl- 4-oxochroman-6-yl)- 1,2,4-oxadiazol- 3-yl)pyridin-2- yl)methanesulfonamide | 442.13 | 443.3 |
| 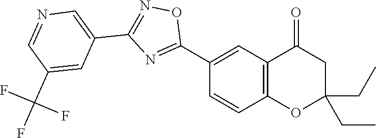 [SMILES: CCC1(CC)CC(=O)c2cc(-c3nc(-c4cncc(C(F)(F)F)c4)no3)ccc2O1] | 557 | 2,2-diethyl-6-(3-(5- (trifluoromethyl)pyridin- 3-yl)-1,2,4-oxadiazol- 5-yl)chroman-4-one | 417.13 | 418 |
| 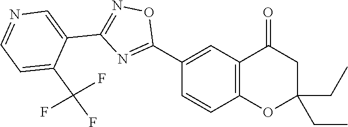 [SMILES: CCC1(CC)CC(=O)c2cc(-c3nc(-c4cnccc4C(F)(F)F)no3)ccc2O1] | 558 | 2,2-diethyl-6-(3-(4- (trifluoromethyl)pyridin- 3-yl)-1,2,4-oxadiazol- 5-yl)chroman-4-one | 417.13 | 418 |
| 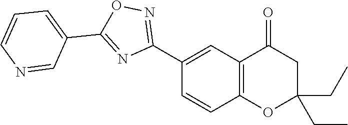 [SMILES: CCC1(CC)CC(=O)c2cc(-c3noc(-c4cccnc4)n3)ccc2O1] | 559 | 2,2-diethyl-6-(5- (pyridin-3-yl)-1,2,4- oxadiazol-3- yl)chroman-4-one | 349.14 | 350 |
| 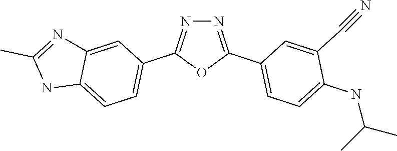 [SMILES: Cc1nc2cc(-c3nnc(-c4ccc(NC(C)C)c(C#N)c4)o3)ccc2[nH]1] | 560 | 5-[5-(2-methy1-1H-1,3- benzodiazol-5-yl)- 1,3,4-oxadiazol- 2-yl]-2-[(propan-2- yl)amino]benzonitrile | 358.15 | 359.2 |
| 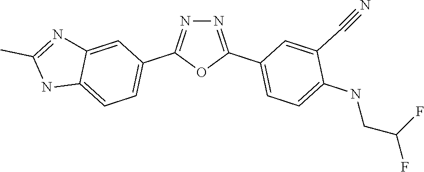 [SMILES: Cc1nc2cc(-c3nnc(-c4ccc(NCC(F)F)c(C#N)c4)o3)ccc2[nH]1] | 561 | 2-[(2,2-difluoro- ethyl)amino]-5-[5- (2-methyl-1H-1,3- benzodiazol-5- yl)-1,3,4-oxadiazol- 2-yl]benzonitrile | 380.12 | 381.2 |
| 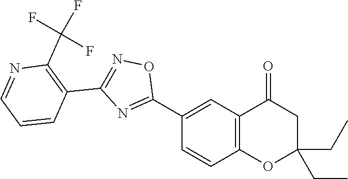 [SMILES: CCC1(CC)CC(=O)c2cc(-c3nc(-c4cccnc4C(F)(F)F)no3)ccc2O1] | 562 | 2,2-diethyl-6-(3-(2- (trifluoromethyl)pyridin- 3-yl)-1,2,4-oxadiazol- 5-yl)chroman-4-one | 417.13 | 418 |
| 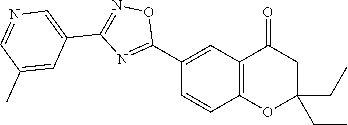 [SMILES: CCC1(CC)CC(=O)c2cc(-c3nc(-c4cncc(C)c4)no3)ccc2O1] | 563 | 2,2-diethyl-6-(3-(5- methylpyridin-3-yl)- 1,2,4-oxadiazol-5- yl)chroman-4-one | 363.16 | 364 |
| 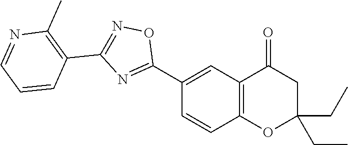 [SMILES: CCC1(CC)CC(=O)c2cc(-c3nc(-c4cccnc4C)no3)ccc2O1] | 564 | 2,2-diethyl-6-(3-(2- methylpyridin-3-yl)- 1,2,4-oxadiazol-5- yl)chroman-4-one | 363.16 | 364 |
| 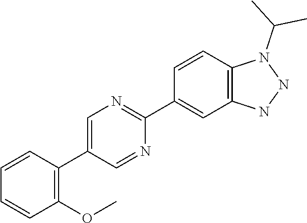 [SMILES: COc1ccccc1-c1cnc(-c2ccc3c(c2)NNN3C(C)C)nc1] | 565 | 1-isopropyl-5-(5-(2- methoxyphenyl)pyrimidin- 2-yl)-1H- benzo[d][1,2,3]triazole | 345.16 | 346.1 |
| 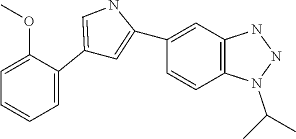 [SMILES: COc1ccccc1-c1c[nH]c(-c2ccc3c(c2)NNN3C(C)C)c1] | 566 | 5-[4-(2-methoxyphenyl)- 1H-pyrrol-2-yl]-1- (propan-2-yl)-1H-1,2,3- benzotriazole | 332.16 | 333.2 |
| 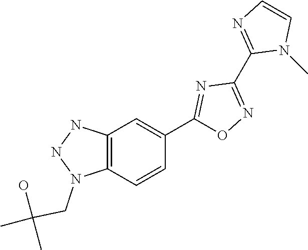 [SMILES: Cn1ccnc1-c1noc(-c2ccc3c(c2)NNN3CC(C)(C)O)n1] | 567 | 2-methyl-1-(5-[3-(1- methyl-1H-imidazol-2- yl)-1,2,4-oxadiazol-5-yl]- 1H-1,2,3-benzotriazol-1- yl}propan-2-ol | 339.14 | 340.1 |
| 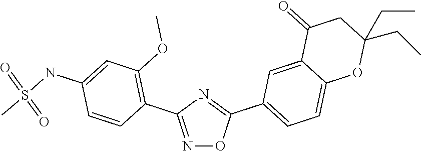 [SMILES: CCC1(CC)CC(=O)c2cc(-c3nc(-c4ccc(NS(C)(=O)=O)cc4OC)no3)ccc2O1] | 568 | N-{4-[5-(2,2-diethyl- 4-oxo-3,4-dihydro- 2H-1-benzopyran-6-yl)- 1,2,4-oxadiazol-3-yl]-3- methoxyphenyl}methane- sulfonamide | 471.15 | 472.1 |
| 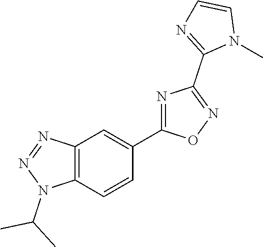 [SMILES: CC(C)n1nnc2cc(-c3nc(-c4nccn4C)no3)ccc21] | 569 | 5-[3-(1-methyl-1H- imidazol-2-yl)-1,2,4- oxadiazol-5-yl]-1- (propan-2-y1)-1H-1,2,3- benzotriazole | 309.13 | 310.1 |
| 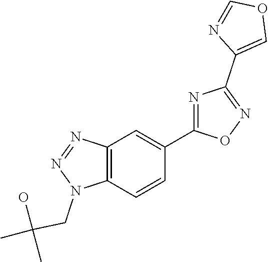 [SMILES: CC(C)(O)Cn1nnc2cc(-c3nc(-c4cocn4)no3)ccc21] | 570 | 2-methyl-1-{5-[3-(1,3- oxazol-4-yl)-1,2,4- oxadiazol-5-yl]-1H- 1,2,3-benzotriazol- 1-yl}propan-2-ol | 326.11 | 327 |
| 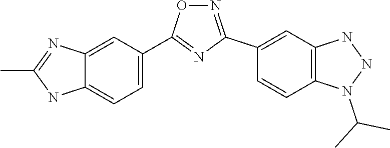 [SMILES: Cc1nc2cc(-c3nc(-c4ccc5c(c4)NNN5C(C)C)no3)ccc2[nH]1] | 571 | 5-[5-(2-methyl-1H-1,3- benzodiazol-5-yl)- 1,2,4-oxadiazol-3-yl]- 1-(propan-2-yl)-1H- 1,2,3-benzotriazole | 359.15 | 360 |
| 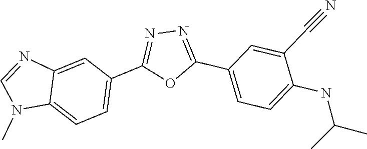 [SMILES: CC(C)Nc1ccc(-c2nnc(-c3ccc4c(c3)ncn4C)o2)cc1C#N] | 572 | 5-[5-(1-methyl-1H- 1,3-benzodiazol-5- yl)-1,3,4-oxadiazol- 2-yl]-2-[(propan-2- yl)amino]benzonitrile | 358.15 | 359.1 |
| 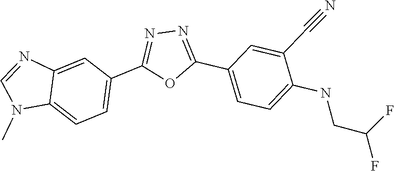 [SMILES: Cn1cnc2cc(-c3nnc(-c4ccc(NCC(F)F)c(C#N)c4)o3)ccc21] | 573 | 2-[(2,2-difluoro- ethyl)amino]-5-[5- (1-methyl-1H-1,3- benzodiazol-5-yl)- 1,3,4-oxadiazol-2- yl]benzonitrile | 380.12 | 381.1 |
| 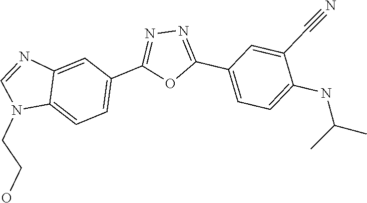 [SMILES: CC(C)Nc1ccc(-c2nnc(-c3ccc4c(c3)ncn4CCO)o2)cc1C#N] | 574 | 5-{5-[1-(2-hydroxy- ethyl)-1H-1,3- benzodiazol-5-yl]- 1,3,4-oxadiazol- 2-yl}-2-[(propan-2- yl)amino]benzonitrile | 388.16 | 389.1 |
| 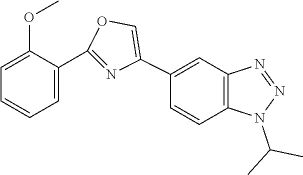 [SMILES: COc1ccccc1-c1nc(-c2ccc3c(c2)nnn3C(C)C)co1] | 575 | 5-[2-(2-methoxy- phenyl)-1,3-oxazol- 4-yl]-1-(propan-2-yl)- 1H-1,2,3-benzotriazole | 334.14 | 335 |
| 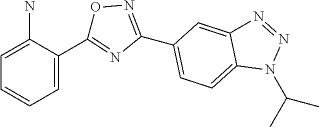 [SMILES: CC(C)n1nnc2cc(-c3noc(-c4ccccc4N)n3)ccc21] | 576 | 2-{3-[1-(propan-2-yl)- 1H-1,2,3-benzotriazol- 5-yl]-1,2,4-oxadiazol- 5-yl}aniline | 320.14 | 321.2 |
| 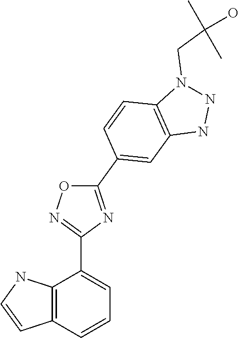 [SMILES: CC(C)(O)CN1NNc2cc(-c3nc(-c4cccc5cc[nH]c45)no3)ccc21] | 577 | 1-{5-[3-(1H-indol-7-yl)- 1,2,4-oxadiazol-5-yl]- 1H-1,2,3-benzotriazol- 1-yl}-2-methylpropan- 2-ol | 374.15 | 375.2 |
| 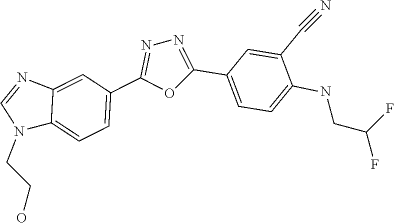 [SMILES: N#Cc1cc(-c2nnc(-c3ccc4c(c3)ncn4CCO)o2)ccc1NCC(F)F] | 578 | 2-[(2,2-difluoro- ethyl)amino]-5-{5-[1- (2-hydroxyethyl)-1H- 1,3-benzodiazol-5- yl]-1,3,4-oxadiazol- 2-yl}benzonitrile | 410.13 | 411.1 |
| 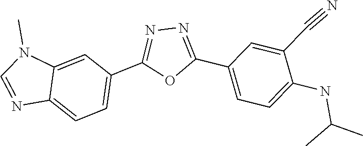 [SMILES: CC(C)Nc1ccc(-c2nnc(-c3ccc4ncn(C)c4c3)o2)cc1C#N] | 579 | 5-[5-(1-methyl-1H-1,3- benzodiazol-6-yl)-1,3,4- oxadiazol-2-yl]- 2-[(propan-2- yl)amino]benzonitrile | 358.15 | 359.2 |
| 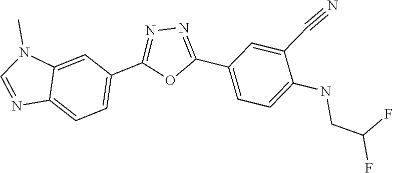 [SMILES: Cn1cnc2ccc(-c3nnc(-c4ccc(NCC(F)F)c(C#N)c4)o3)cc21] | 580 | 2-[(2,2-difluoro- ethyl)amino]-5-[5- (1-methyl-1H-1,3- benzodiazol-6-yl)- 1,3,4-oxadiazol-2- yl]benzonitrile | 380.12 | 381.1 |
| 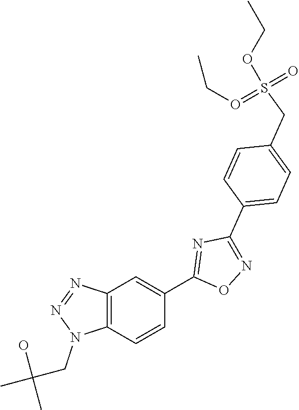 | 581 | [(4-{5-[1-(2-hydroxy-2- methylpropyl)-1H-1,2,3- benzotriazol-5-yl]- 1,2,4-oxadiazol-3-yl} phenyl)methyl] phosphonate | 485.18 | 486.2 |
| 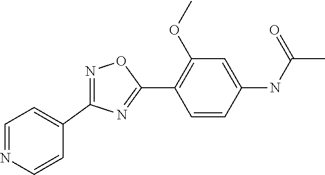 [SMILES: COc1cc(NC(C)=O)ccc1-c1nc(-c2ccncc2)no1] | 582 | N-{3-methoxy-4-[3- (pyridin-4-yl)- 1,2,4-oxadiazol-5- yl]phenyl}acetamide | 310.11 | 311.2 |
| 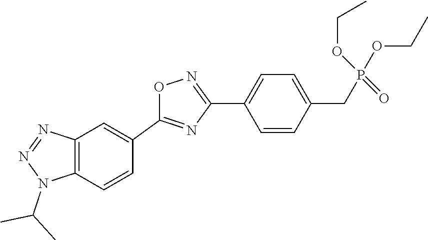 [SMILES: CCOP(=O)(Cc1ccc(-c2noc(-c3ccc4c(c3)nnn4C(C)C)n2)cc1)OCC] | 583 | diethyl[(4-{5-[1-(propan- 2-yl)-1H-1,2,3- benzotriazol-5-yl]-1,2,4- oxadiazol-3-yl}phenyl) methyl]phosphonate | 455.17 | 456.1 |
| 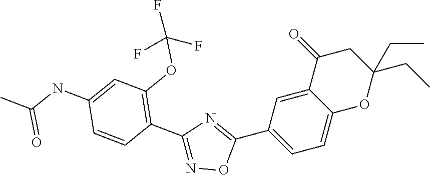 [SMILES: CCC1(CC)CC(=O)c2cc(-c3nc(-c4ccc(NC(C)=O)cc4OC(F)(F)F)no3)ccc2O1] | 584 | N-{4-[5-(2,2-diethyl- 4-oxo-3,4-dihydro-2H- 1-benzopyran-6-yl)- 1,2,4-oxadiazol-3-yl]-3- (trifluoromethoxy) phenyl}acetamide | 489.15 | 490.1 |
| 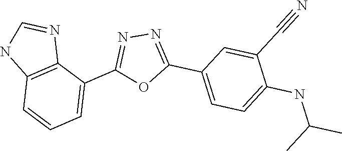 [SMILES: CC(C)Nc1ccc(-c2nnc(-c3cccc4[nH]cnc34)o2)cc1C#N] | 585 | 5-[5-(1H-1,3-benzo- diazol-4-yl)-1,3,4- oxadiazol-2-yl]-2- [(propan-2- yl)amino]benzonitrile | 344.14 | 345.1 |
| 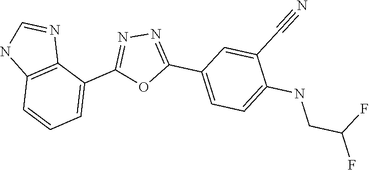 [SMILES: N#Cc1cc(-c2nnc(-c3cccc4[nH]cnc34)o2)ccc1NCC(F)F] | 586 | 5-[5-(1H-1,3- benzodiazol-4-yl)- 1,3,4-oxadiazol-2- yl]-2-[(2,2-difluoro- ethyl)amino]benzonitrile | 366.1 | 367.1 |
| 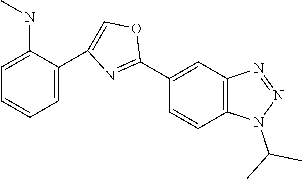 [SMILES: CNc1ccccc1-c1coc(-c2ccc3c(c2)nnn3C(C)C)n1] | 587 | N-methyl-2-{2-[1- (propan-2-yl)-1H- 1,2,3-benzotriazol-5- yl]-1,3-oxazol-4- yl}aniline | 333.16 | 334 |
| 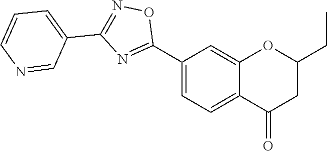 [SMILES: CCC1CC(=O)c2ccc(-c3nc(-c4cccnc4)no3)cc2O1] | 588 | 2-ethyl-7-[3-(pyridin- 3-yl)-1,2,4-oxadiazol- 5-yl]-3,4-dihydro-2H-1- benzopyran-4-one | 321.11 | 322.1 |
| 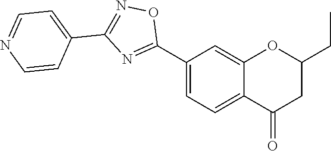 [SMILES: CCC1CC(=O)c2ccc(-c3nc(-c4ccncc4)no3)cc2O1] | 589 | 2-ethyl-7-[3-(pyridin-4- yl)-1,2,4-oxadiazol- 5-yl]-3,4-dihydro-2H- 1-benzopyran-4-one | 321.11 | 322.1 |
| 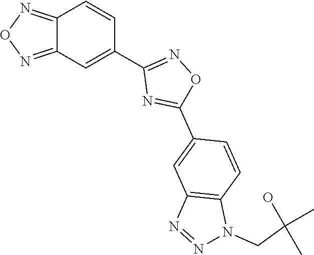 [SMILES: CC(C)(O)Cn1nnc2cc(-c3nc(-c4ccc5nonc5c4)no3)ccc21] | 590 | 1-{5-[3-(2,1,3-benzoxa- diazol-5-yl)-1,2,4- oxadiazol-5-yl]-1H- 1,2,3-benzotriazol-1- yl}-2-methylpropan- 2-ol | 377.12 | 378.1 |
| 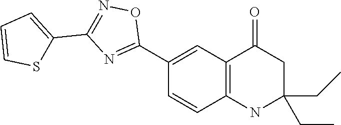 [SMILES: CCC1(CC)CC(=O)c2cc(-c3nc(-c4cccs4)no3)ccc2N1] | 591 | 2,2-diethyl-6-(3- (thiophen-2-yl)-1,2,4- oxadiazol-5-yl)-2,3- dihydroquinolin- 4(1H)-one | 353.12 | 353.9 |
| 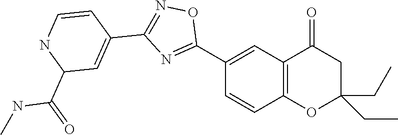 [SMILES: CCC1(CC)CC(=O)c2cc(-c3nc(C4=CC(C(=O)NC)NC=C4)no3)ccc2O1] | 592 | 4-(5-(2,2-diethyl-4- oxochroman-6-yl)- 1,2,4-oxadiazol-3-yl)- N-methylpicolinamide | 406.16 | 407 |
| 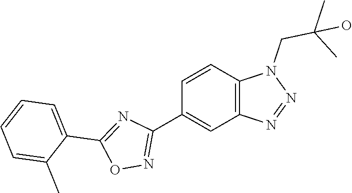 [SMILES: Cc1ccccc1-c1nc(-c2ccc3c(c2)nnn3CC(C)(C)O)no1] | 593 | 2-methyl-1-(5-(5-(o- tolyl)-1,2,4- oxadiazol-3-yl)-1H- benzo[d][1,2,3]triazol- 1-yl)propan-2-ol | 349.15 | 350 |
| 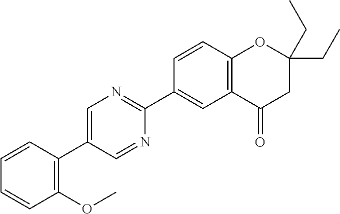 [SMILES: CCC1(CC)CC(=O)c2cc(-c3ncc(-c4ccccc4OC)cn3)ccc2O1] | 594 | 2,2-diethyl-6-(5-(2- methoxyphenyl)pyrimidin- 2-yl)chroman-4-one | 388.18 | 389 |
| 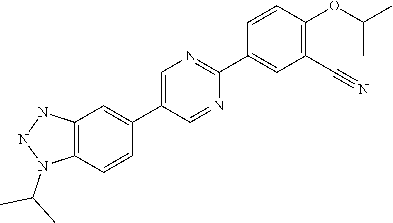 [SMILES: CC(C)Oc1ccc(-c2ncc(-c3ccc4c(c3)NNN4C(C)C)cn2)cc1C#N] | 595 | 2-isopropoxy-5-(5-(1- isopropyl-1H- benzo[d][1,2,3]triazol- 5-yl)pyrimidin- 2-yl)benzonilrile | 398.19 | 399.2 |
| 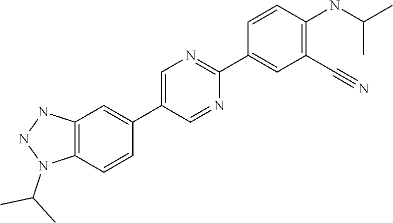 [SMILES: CC(C)Nc1ccc(-c2ncc(-c3ccc4c(c3)NNN4C(C)C)cn2)cc1C#N] | 596 | 5-(5-(1-isopropyl-1H- benzo[d][1,2,3]triazol- 5-yl)pyrimidin-2-yl)- 2-(isopropyl- amino)benzonitrile | 397.2 | 398.1 |
| 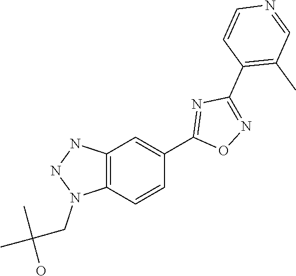 [SMILES: Cc1cnccc1-c1noc(-c2ccc3c(c2)NNN3CC(C)(C)O)n1] | 597 | 2-methyl-1-{5-[3- (3-methylpyridin- 4-yl)-1,2,4-oxadiazol- 5-yl]-1H-1,2,3- benzotriazol-1- yl}propan-2-ol | 350.15 | 351.1 |
| 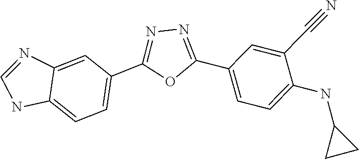 [SMILES: N#Cc1cc(-c2nnc(-c3ccc4[nH]cnc4c3)o2)ccc1NC1CC1] | 598 | 5-[5-(1H-1,3-benzo- diazol-5-yl)-1,3,4- oxadiazol-2-yl]-2- (cyclopropyl- amino)benzonitrile | 342.12 | 343.1 |
| 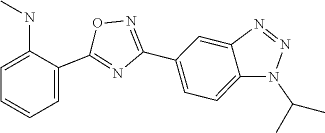 [SMILES: CNc1ccccc1-c1nc(-c2ccc3c(c2)nnn3C(C)C)no1] | 599 | N-methyl-2-{3-[1- (propan-2-yl)-1H- l,2,3-benzotriazol-5- yl]-1,2,4-oxadiazol- 5-yl}aniline | 334.15 | 335.2 |
| 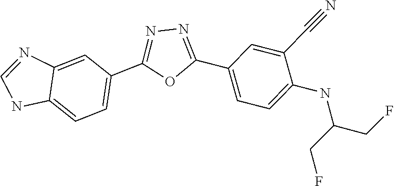 [SMILES: N#Cc1cc(-c2nnc(-c3ccc4[nH]cnc4c3)o2)ccc1NC(CF)CF] | 600 | 5-[5-(1H-1,3-benzo- diazol-5-yl)-1,3,4- oxadiazol-2-yl]-2-[(1,3- difluoropropan-2- yl)amino]benzonitrile | 380.12 | 381.1 |
| 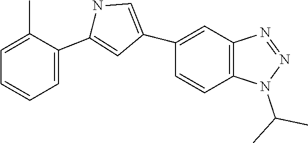 [SMILES: Cc1ccccc1-c1cc(-c2ccc3c(c2)nnn3C(C)C)c[nH]1] | 601 | 5-[5-(2-methylphenyl)- 1H-pyrrol-3-yl]-1- (propan-2-yl)-1H-1,2,3- benzotriazole | 316.17 | 317 |
| 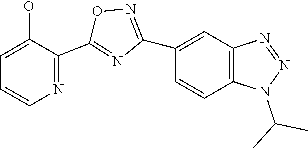 [SMILES: CC(C)n1nnc2cc(-c3noc(-c4ncccc4O)n3)ccc21] | 602 | 2-{3-[1-(propan-2- yl)-1H-1,2,3-benzo- triazol-5-yl]-1,2,4- oxadiazol-5- yl}pyridin-3-ol | 322.12 | 323 |
| 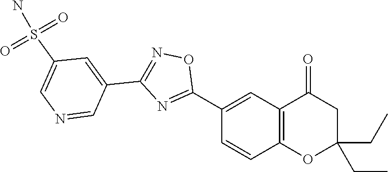 [SMILES: CCC1(CC)CC(=O)c2cc(-c3nc(-c4cncc(S(N)(=O)=O)c4)no3)ccc2O1] | 603 | 5-(5-(2,2-diethyl-4- oxochroman-6-yl)-1,2,4- oxadiazol-3-yl)pyridine- 3-sulfonamide | 428.12 | 428.9 |
| 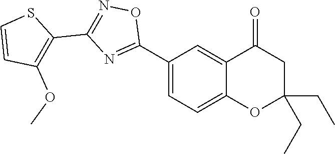 [SMILES: CCC1(CC)CC(=O)c2cc(-c3nc(-c4sccc4OC)no3)ccc2O1] | 604 | 2,2-diethyl-6-(3-(3- methoxythiophen-2-yl)- 1,2,4-oxadiazol-5- yl)chroman-4-one | 384.11 | 385.2 |
| 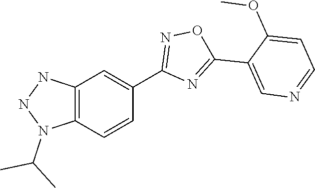 [SMILES: COc1ccncc1-c1nc(-c2ccc3c(c2)NNN3C(C)C)no1] | 605 | 3-(1-isopropyl-1H- benzo[d][1,2,3]triazol- 5-yl)-5-(4-methoxy- pyridin-3-yl)-1,2,4- oxadiazole | 336.13 | 337.2 |
| 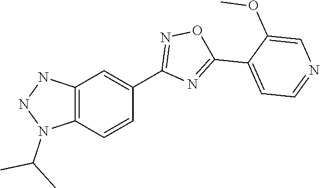 [SMILES: COc1cnccc1-c1nc(-c2ccc3c(c2)NNN3C(C)C)no1] | 606 | 3-(1-isopropyl-1H- benzo[d][1,2,3]triazol- 5-yl)-5-(3-methoxy- pyridin-4-yl)-1,2,4- oxadiazole | 336.13 | 337.2 |
| 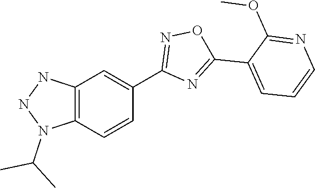 [SMILES: COc1ncccc1-c1nc(-c2ccc3c(c2)NNN3C(C)C)no1] | 607 | 3-(1-isopropyl-1H- benzo[d][1,2,3]triazol- 5-yl)-5-(2-methoxy- pyridin-3-yl)-1,2,4- oxadiazole | 336.13 | 337.2 |
| 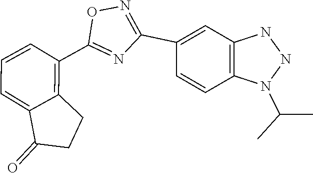 [SMILES: CC(C)N1NNc2cc(-c3noc(-c4cccc5c4CCC5=O)n3)ccc21] | 608 | 4-{3-[1-(propan-2- yl)-1H-1,2,3-benzo- triazol-5-yl]-1,2,4- oxadiazol-5-yl}-2,3- dihydro-1H-inden- 1-one | 359.14 | 360 |
| 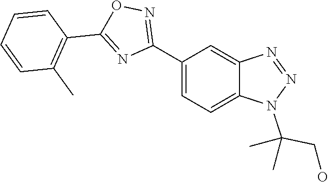 [SMILES: Cc1ccccc1-c1nc(-c2ccc3c(c2)nnn3C(C)(C)CO)no1] | 609 | 2-methyl-2-(5-(5-(o- tolyl)-1,2,4-oxadiazol- 3-yl)-1H-benzo[d] [1,2,3]triazol-1- yl)propan-1-ol | 349.15 | 350.1 |
| 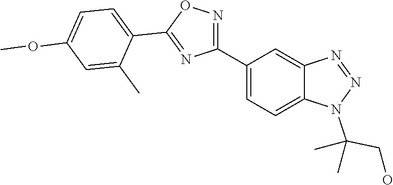 [SMILES: COc1ccc(-c2nc(-c3ccc4c(c3)nnn4C(C)(C)CO)no2)c(C)c1] | 610 | 2-(5-(5-(4-methoxy- 2-methylphenyl)- 1,2,4-oxadiazol-3- yl)-1H-benzo[d] [1,2,3]triazol-1-yl)- 2-methylpropan-1-ol | 379.16 | 380 |
| 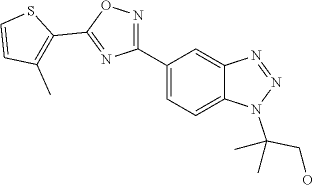 [SMILES: Cc1ccsc1-c1nc(-c2ccc3c(c2)nnn3C(C)(C)CO)no1] | 611 | 2-methyl-2-(5-(5-(3- methylthiophen-2-yl)- 1,2,4-oxadiazol-3- yl)-1H-benzo[d] [1,2,3]triazol-1- yl)propan-1-ol | 355.11 | 355.9 |
| 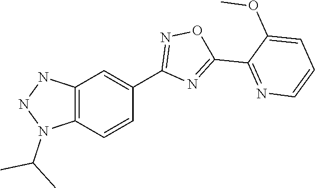 [SMILES: COc1cccnc1-c1nc(-c2ccc3c(c2)NNN3C(C)C)no1] | 612 | 3-(1-isopropyl-1H- benzo[d][1,2,3]triazol- 5-yl)-5-(3-methoxy- pyridin-2-yl)-1,2,4- oxadiazole | 336.13 | 337.2 |
| 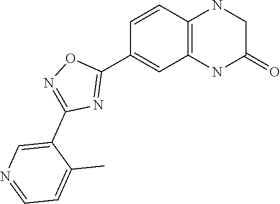 [SMILES: Cc1ccncc1-c1noc(-c2ccc3c(c2)NC(=O)CN3)n1] | 613 | 7-[3-(4-methylpyridin- 3-yl)-1,2,4-oxadiazol- 5-yl]-1,2,3,4-tetra- hydroquinoxalin-2-one | 307.11 | 307.1 |
| 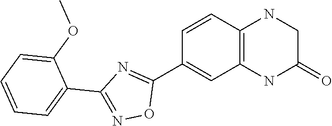 [SMILES: COc1ccccc1-c1noc(-c2ccc3c(c2)NC(=O)CN3)n1] | 614 | 7-[3-(2-methoxyphenyl)- 1,2,4-oxadiazol-5-yl]- 1,2,3,4-tetrahydro- quinoxalin-2-one | 322.11 | 322.1 |
| 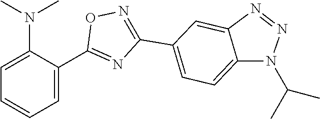 [SMILES: CC(C)n1nnc2cc(-c3noc(-c4ccccc4N(C)C)n3)ccc21] | 615 | N,N-dimethy1-2-{3- [1-(propan-2-yl)-1H- 1,2,3-benzotriazol-5- yl]-1,2,4-oxadiazol- 5-yl}aniline | 348.17 | 349.1 |
| 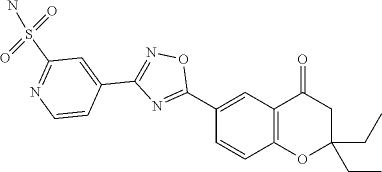 [SMILES: CCC1(CC)CC(=O)c2cc(-c3nc(-c4ccnc(S(N)(=O)=O)c4)no3)ccc2O1] | 616 | 4-(5-(2,2-diethyl-4- oxochroman-6-yl)-1,2,4- oxadiazol-3-yl)pyridine- 2-sulfonamide | 428.12 | 428.9 |
| 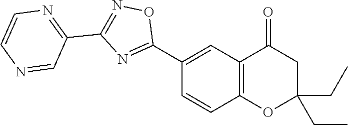 [SMILES: CCC1(CC)CC(=O)c2cc(-c3nc(-c4cnccn4)no3)ccc2O1] | 617 | 2,2-diethyl-6-(3- (pyrazin-2-yl)- 1,2,4-oxadiazol-5- yl)chroman-4-one | 350.14 | 351.1 |
| 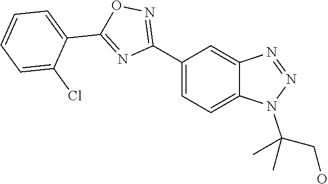 [SMILES: CC(C)(CO)n1nnc2cc(-c3noc(-c4ccccc4Cl)n3)ccc21] | 618 | 2-(5-(5-(2-chloro- phenyl)-1,2,4- oxadiazol-3-yl)-1H- benzo[d][1,2,3]triazol- 1-yl)-2- methylpropan-1-ol | 369.1 | 369.9 |
| 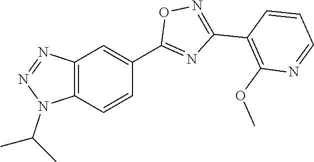 [SMILES: COc1ncccc1-c1noc(-c2ccc3c(c2)nnn3C(C)C)n1] | 619 | 5-(1-isopropyl-1H- benzo[d][1,2,3]triazol- 5-yl)-3-(2-methoxy- pyridin-3-yl)-1,2,4- oxadiazole | 336.13 | 337.2 |
| 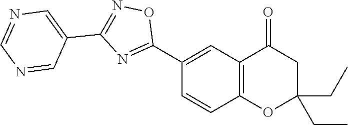 [SMILES: CCC1(CC)CC(=O)c2cc(-c3nc(-c4cncnc4)no3)ccc2O1] | 620 | 2,2-diethyl-6-(3- (pyrimidin-5-yl)- 1,2,4-oxadiazol-5- yl)chroman-4-one | 350.14 | 351.1 |
| 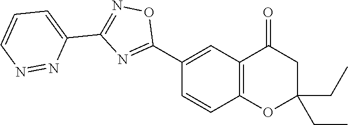 [SMILES: CCC1(CC)CC(=O)c2cc(-c3nc(-c4cccnn4)no3)ccc2O1] | 621 | 2,2-diethyl-6-(3- (pyridazin-3-yl)- 1,2,4-oxadiazol-5- yl)chroman-4-one | 350.14 | 351.1 |
| 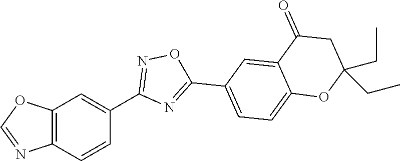 [SMILES: CCC1(CC)CC(=O)c2cc(-c3nc(-c4ccc5ncoc5c4)no3)ccc2O1] | 622 | 6-(3-(benzo[d]oxazol- 6-yl)-1,2,4-oxadiazol- 5-yl)-2,2-diethyl- chroman-4-one | 389.14 | 390 |
| 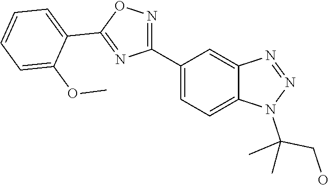 [SMILES: COc1ccccc1-c1nc(-c2ccc3c(c2)nnn3C(C)(C)CO)no1] | 623 | 2-(5-(5-(2-methoxy- phenyl)-1,2,4- oxadiazol-3-yl)-1H- benzo[d][1,2,3]triazol- 1-yl)-2-methyl- propan-1-ol | 365.15 | 365.9 |
| 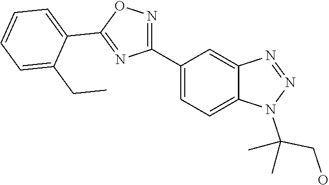 [SMILES: CCc1ccccc1-c1nc(-c2ccc3c(c2)nnn3C(C)(C)CO)no1] | 624 | 2-(5-(5-(2-ethyl- phenyl)-1,2,4- oxadiazol-3-yl)-1H- benzo[d][1,2,3]triazol- 1-yl)-2-methyl- propan-1-ol | 363.17 | 364.1 |
| 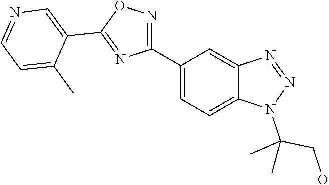 [SMILES: Cc1ccncc1-c1nc(-c2ccc3c(c2)nnn3C(C)(C)CO)no1] | 625 | 2-methyl-2-(5-(5- (4-methylpyridin- 3-yl)-1,2,4-oxadiazol- 3-yl)-1H-benzo[d] [1,2,3]triazol-1- yl)propan-1-ol | 350.15 | 351.1 |
| 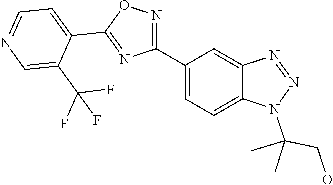 [SMILES: CC(C)(CO)n1nnc2cc(-c3noc(-c4ccncc4C(F)(F)F)n3)ccc21] | 626 | 2-methyl-2-(5-(5- (3-(trifluoro- methyl)pyridin-4- yl)-1,2,4-oxadiazol- 3-yl)-1H-benzo[d] [1,2,3]triazol-1- yl)propan-1-ol | 404.12 | 405.1 |
| 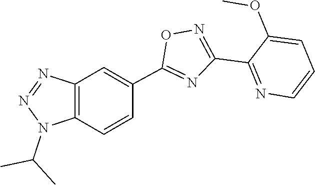 [SMILES: COc1cccnc1-c1noc(-c2ccc3c(c2)nnn3C(C)C)n1] | 627 | 5-(1-isopropyl-1H- benzo[d][1,2,3]triazol- 5-yl)-3-(3- methoxypyridin-2- yl)-1,2,4-oxadiazole | 336.13 | 337.1 |
| 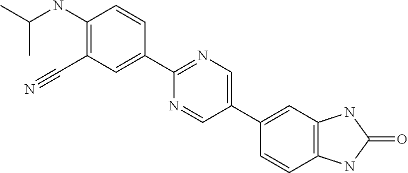 [SMILES: CC(C)Nc1ccc(-c2ncc(-c3ccc4[nH]c(=O)[nH]c4c3)cn2)cc1C#N] | 628 | 2-(isopropylamino)-5- (5-(2-oxo-2,3-dihydro- 1H-benzo[d]imidazol- 5-yl)pyrimidin-2- yl)benzonitrile | 370.15 | 371.1 |
| 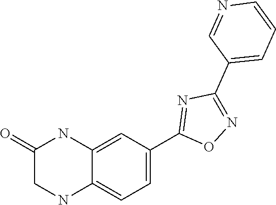 [SMILES: O=C1CNc2ccc(-c3nc(-c4cccnc4)no3)cc2N1] | 629 | 7-[3-(pyridin-3-yl)- 1,2,4-oxadiazol- 5-yl]-1,2,3,4-tetra- hydroquinoxalin-2-one | 293.09 | 292.1 |
| 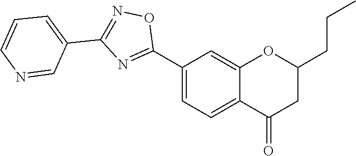 [SMILES: CCCC1CC(=O)c2ccc(-c3nc(-c4cccnc4)no3)cc2O1] | 630 | 2-propyl-7-[3-(pyridin-3- yl)-1,2,4-oxadiazol-5- yl]-3,4-dihydro-2H-1- benzopyran-4-one | 335.13 | 336.1 |
| 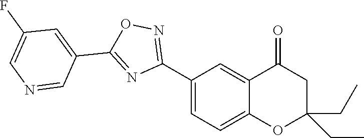 [SMILES: CCC1(CC)CC(=O)c2cc(-c3noc(-c4cncc(F)c4)n3)ccc2O1] | 631 | 2,2-diethyl-6-[5-(5- fluoropyridin-3-yl)- 1,2,4-oxadiazol-3- yl]-3,4-dihydro-2H- 1-benzopyran-4-one | 367.13 | 368.1 |
| 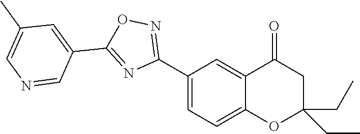 [SMILES: CCC1(CC)CC(=O)c2cc(-c3noc(-c4cncc(C)c4)n3)ccc2O1] | 632 | 2,2-diethyl-6-[5- (5-methylpyridin-3- yl)-1,2,4-oxadiazol- 3-yl]-3,4-dihydro-2H- 1-benzopyran-4-one | 363.16 | 364.1 |
| 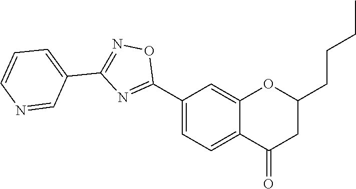 [SMILES: CCCCC1CC(=O)c2ccc(-c3nc(-c4cccnc4)no3)cc2O1] | 633 | 2-butyl-7-[3-(pyridin- 3-yl)-1,2,4-oxadiazol-5- yl]-3,4-dihydro-2H-1- benzopyran-4-one | 349.14 | 350.1 |
| 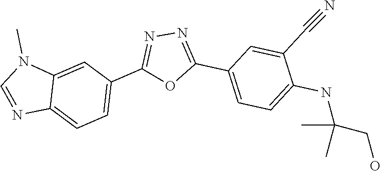 [SMILES: Cn1cnc2ccc(-c3nnc(-c4ccc(NC(C)(C)CO)c(C#N)c4)o3)cc21] | 634 | 2-[(1-hydroxy-2- methylpropan-2- yl)amino]-5-[5-(1- methyl-1H-1,3- benzodiazol-6-yl)- 1,3,4-oxadiazol-2- yl]benzonitrile | 388.16 | 389.2 |
| 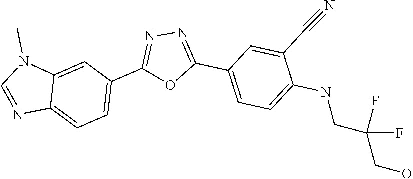 [SMILES: Cn1cnc2ccc(-c3nnc(-c4ccc(NCC(F)(F)CO)c(C#N)c4)o3)cc21] | 635 | 2-[(2,2-difluoro-3- hydroxypropyl)amino]- 5-[5-(1-methyl-1H- 1,3-benzodiazol-6-yl)- 1,3,4-oxadiazol-2- yl]benzonitrile | 410.13 | 411.2 |
| 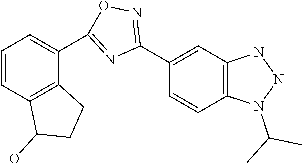 [SMILES: CC(C)N1NNc2cc(-c3noc(-c4cccc5c4CCC5O)n3)ccc21] | 636 | 4-{3-[1-(propan-2-yl)- 1H-1,2,3-benzotriazol- 5-yl]-1,2,4-oxadiazol- 5-yl}-2,3-dihydro-1H- inden-1-ol | 361.15 | 362 |
| 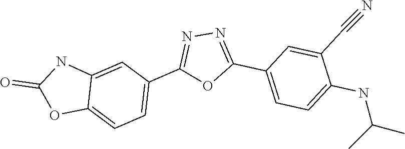 [SMILES: CC(C)Nc1ccc(-c2nnc(-c3ccc4oc(=O)[nH]c4c3)o2)cc1C#N] | 637 | 5-[5-(2-oxo-2,3-dihydro- 1,3-benzoxazol-5-yl)- 1,3,4-oxadiazol- 2-yl]-2-[(propan-2- yl)amino]benzonitrile | 361.12 | 362.2 |
| 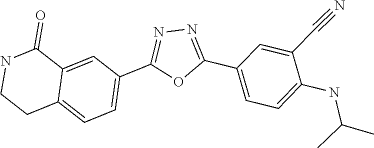 [SMILES: CC(C)Nc1ccc(-c2nnc(-c3ccc4c(c3)C(=O)NCC4)o2)cc1C#N] | 638 | 5-[5-(1-oxo-1,2,3,4- tetrahydroisoquinolin- 7-yl)-1,3,4-oxadiazol- 2-yl]-2-[(propan-2- yl)amino]benzonitrile | 373.15 | 374.2 |
| 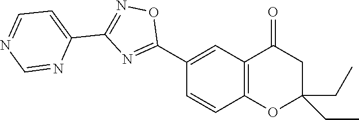 [SMILES: CCC1(CC)CC(=O)c2cc(-c3nc(-c4ccncn4)no3)ccc2O1] | 639 | 2,2-diethyl-6-(3- (pyrimidin-4-yl)-1,2,4- oxadiazol-5- yl)chroman-4-one | 350.14 | 351.1 |
| 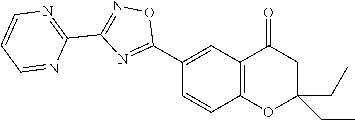 [SMILES: CCC1(CC)CC(=O)c2cc(-c3nc(-c4ncccn4)no3)ccc2O1] | 640 | 2,2-diethyl-6-(3- (pyrimidin-2-yl)-1,2,4- oxadiazol-5-yl)chroman- 4-one | 350.14 | 351.1 |
| 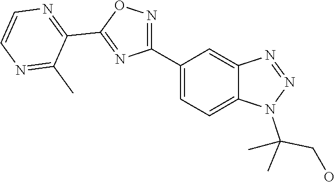 [SMILES: Cc1nccnc1-c1nc(-c2ccc3c(c2)nnn3C(C)(C)CO)no1] | 641 | 2-methyl-2-(5-(5-(3- methyl-pyrazin-2-yl)- 1,2,4-oxadiazol-3-yl)-1H- benzo[d][1,2,3]triazol- 1-yl)propan-1-ol | 351.14 | 352.2 |
| 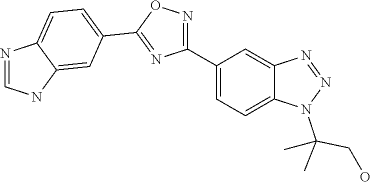 [SMILES: CC(C)(CO)n1nnc2cc(-c3noc(-c4ccc5nc[nH]c5c4)n3)ccc21] | 642 | 2-(5-(5-(1H- benzo[d]imidazol-6- yl)-1,2,4-oxadiazol-3- yl)-1H-benzo[d][1,2,3] triazol-1-yl)-2- methylpropan-1-ol | 375.14 | 376.1 |
| 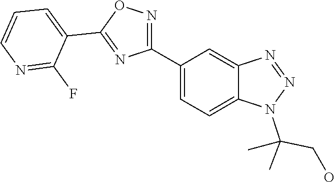 [SMILES: CC(C)(CO)n1nnc2cc(-c3noc(-c4cccnc4F)n3)ccc21] | 643 | 2-(5-(5-(2-fluoro- pyridin-3-yl)-1,2,4- oxadiazol-3-yl)-1H- benzo[d][1,2,3]triazol- 1-yl)-2-methylpropan- 1-ol | 354.12 | 355.1 |
| 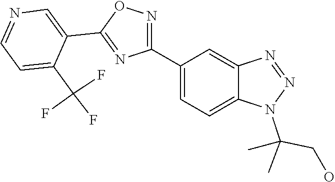 [SMILES: CC(C)(CO)n1nnc2cc(-c3noc(-c4cnccc4C(F)(F)F)n3)ccc21] | 644 | 2-methyl-2-(5-(5- (4-(trifluoro- methyl)pyridin-3-yl)- 1,2,4-oxadiazol-3- yl)-1H-benzo[d][1,2,3] triazol-1-yl)propan-1-ol | 404.12 | 405.2 |
| 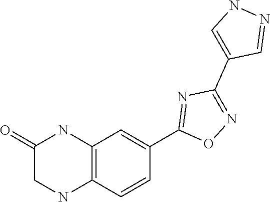 [SMILES: O=C1CNc2ccc(-c3nc(-c4cn[nH]c4)no3)cc2N1] | 645 | 7-[3-(1H-pyrazol-4-yl)- 1,2,4-oxadiazol-5- yl]-1,2,3,4-tetrahydro- quinoxalin-2-one | 282.09 | 281.1 |
| 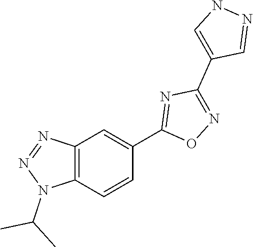 [SMILES: CC(C)n1nnc2cc(-c3nc(-c4cn[nH]c4)no3)ccc21] | 646 | 1-(propan-2-yl)-5-[3- (1H-pyrazol-4-yl)- 1,2,4-oxadiazol-5-yl]- 1H-1,2,3-benzotriazole | 295.12 | 296.2 |
| 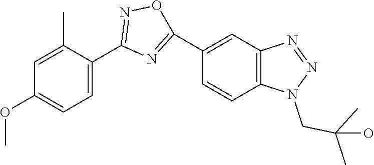 [SMILES: COc1ccc(-c2noc(-c3ccc4c(c3)nnn4CC(C)(C)O)n2)c(C)c1] | 647 | 1-{5-[3-(4-methoxy-2- methylphenyl)-1,2,4- oxadiazol-5-yl]-1H- 1,2,3-benzotriazol-1- yl}-2-methylpropan- 2-ol | 379.16 | 380.1 |
| 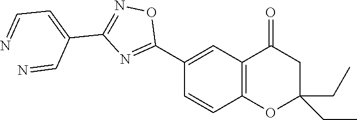 [SMILES: CCC1(CC)CC(=O)c2cc(-c3nc(/C(C=N)=C/C=N)no3)ccc2O1] | 648 | 2,2-diethyl-6-(3- (pyridazin-4-yl)-1,2,4- oxadiazol-5-yl)chroman- 4-one | 350.14 | 351 |
| 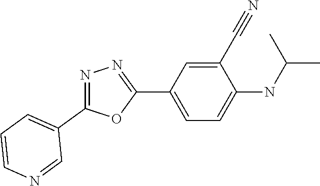 [SMILES: CC(C)Nc1ccc(-c2nnc(-c3cccnc3)o2)cc1C#N] | 649 | 2-(isopropylamino)-5- (5-(pyridin-3-yl)- 1,3,4-oxadiazol-2- yl)benzonitrile | 305.13 | 306.3 |
| 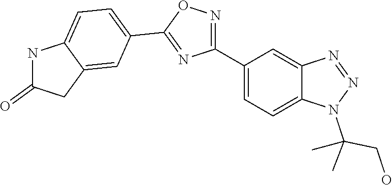 [SMILES: CC(C)(CO)n1nnc2cc(-c3noc(-c4ccc5c(c4)CC(=O)N5)n3)ccc21] | 650 | 5-(3-(1-(1-hydroxy-2- methylpropan-2-yl)-1H- benzo[d][1,2,3]triazol- 5-yl)-1,2,4-oxadiazol- 5-yl)indolin-2-one | 390.14 | 391.1 |
| 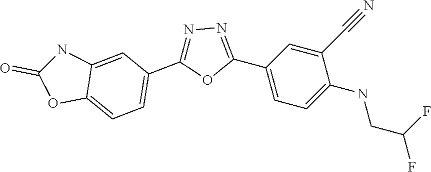 [SMILES: N#Cc1cc(-c2nnc(-c3ccc4oc(=O)[nH]c4c3)o2)ccc1NCC(F)F] | 651 | 2-[(2,2-difluoro- ethyl)amino]-5-[5- (2-oxo-2,3-dihydro- 1,3-benzoxazol-5- yl)-1,3,4-oxadiazol- 2-yl]benzonitrile | 383.08 | 384 |
| 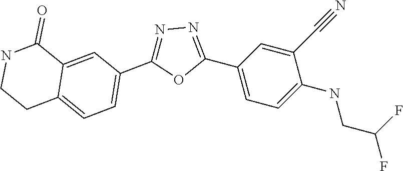 [SMILES: N#Cc1cc(-c2nnc(-c3ccc4c(c3)C(=O)NCC4)o2)ccc1NCC(F)F] | 652 | 2-[(2,2-difluoro- ethyl)amino]-5-[5- (1-oxo-1,2,3,4- tetrahydroisoquinolin- 7-yl)-1,3,4-oxadiazol- 2-yl]benzonitrile | 395.12 | 396.1 |
| 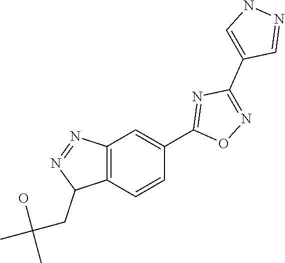 [SMILES: CC(C)(O)CC1N=Nc2cc(-c3nc(-c4cn[nH]c4)no3)ccc21] | 653 | 2-methyl-1-{6-[3-(1H- pyrazol-4-yl)-1,2,4- oxadiazol-5-yl]-3H- indazol-3-yl}propan- 2-ol | 324.13 | 326.2 |
| 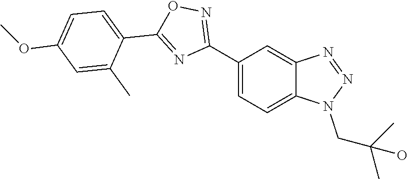 [SMILES: COc1ccc(-c2nc(-c3ccc4c(c3)nnn4CC(C)(C)O)no2)c(C)c1] | 654 | 1-(5-(5-(4-methoxy-2- methylphenyl)-1,2,4- oxadiazol-3-yl)-1H- benzo[d][1,2,3]triazol- 1-yl)-2-methylpropan- 2-ol | 379.16 | 380.3 |
| 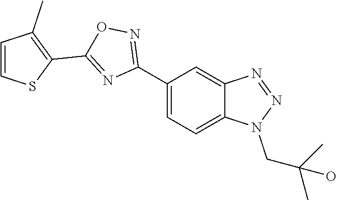 [SMILES: Cc1ccsc1-c1nc(-c2ccc3c(c2)nnn3CC(C)(C)O)no1] | 655 | 2-methyl-1-(5-(5-(3- methylthiophen-2- yl)-1,2,4-oxadiazol- 3-yl)-1H-benzo[d] [1,2,3]triazol-1- yl)propan-2-ol | 355.11 | 356.2 |
| 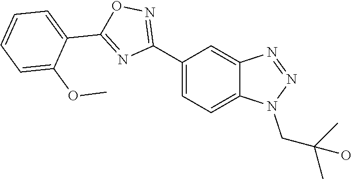 [SMILES: COc1ccccc1-c1nc(-c2ccc3c(c2)nnn3CC(C)(C)O)no1] | 656 | 1-(5-(5-(2-methoxy- phenyl)-1,2,4- oxadiazol-3-yl)-1H- benzo[d][1,2,3]triazol- 1-yl)-2-methyl- propan-2-ol | 365.15 | 366.2 |
| 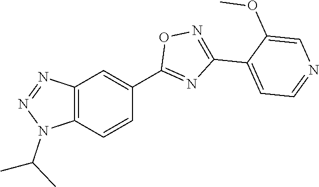 [SMILES: COc1cnccc1-c1noc(-c2ccc3c(c2)nnn3C(C)C)n1] | 657 | 5-(1-isopropyl-1H- benzo[d][1,2,3]triazol- 5-yl)-3-(3-methoxy- pyridin-4-yl)-1,2,4- oxadiazole | 336.13 | 337.1 |
| 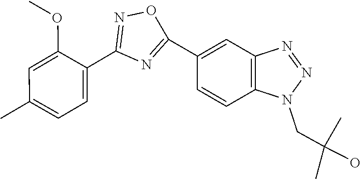 [SMILES: COc1cc(C)ccc1-c1noc(-c2ccc3c(c2)nnn3CC(C)(C)O)n1] | 662 | 1-{5-[3-(2-methoxy-4- methylphenyl)-1,2,4- oxadiazol-5-yl]-1H- 1,2,3-benzotriazol- 1-yl}-2-methyl- propan-2-ol | 379.16 | 380 |
| 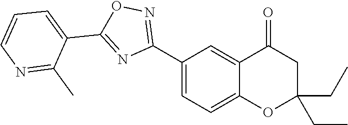 [SMILES: CCC1(CC)CC(=O)c2cc(-c3noc(-c4cccnc4C)n3)ccc2O1] | 663 | 2,2-diethyl-6-[5-(2- methylpyridin-3-yl)- 1,2,4-oxadiazol-3- yl]-3,4-dihydro-2H- 1-benzopyran-4-one | 363.16 | 364.1 |
| 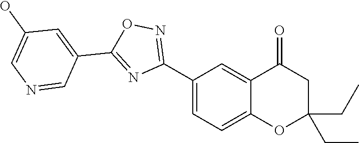 [SMILES: CCC1(CC)CC(=O)c2cc(-c3noc(-c4cncc(O)c4)n3)ccc2O1] | 664 | 2,2-diethyl-6-[5-(5- hydroxypyridin-3- yl)-1,2,4-oxadiazol-3- yl]-3,4-dihydro-2H-1- benzopyran-4-one | 365.14 | 366.1 |
| 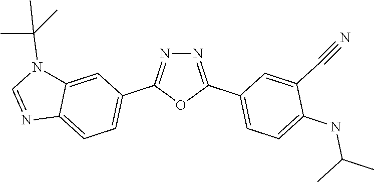 [SMILES: CC(C)Nc1ccc(-c2nnc(-c3ccc4ncn(C(C)(C)C)c4c3)o2)cc1C#N] | 665 | 5-[5-(1-tert-buty1- 1H-1,3-benzodiazol-6- yl)-1,3,4-oxadiazol- 2-yl]-2-[(propan-2- yl)amino]benzonitrile | 400.2 | 401.3 |
| 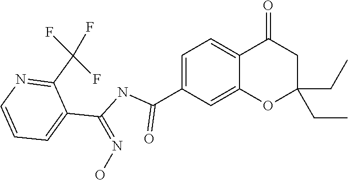 [SMILES: CCC1(CC)CC(=O)c2ccc(C(=O)N/C(=N/O)c3cccnc3C(F)(F)F)cc2O1] | 666 | 2,2-diethyl-N-[(1E)- (hydroxyimino)[2- (trifluoromethyl)pyridin- 3-yl]methyl]-4-oxo- 3,4-dihydro-2H- 1-benzopyran-7- carboxamide | 435.14 | 436.2 |
| 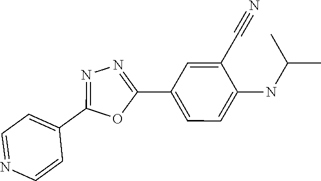 [SMILES: CC(C)Nc1ccc(-c2nnc(-c3ccncc3)o2)cc1C#N] | 667 | 2-(isopropylamino)- 5-(5-(pyridin-4- yl)-l,3,4-oxadiazol-2- yl)benzonitrile | 305.13 | 306.3 |
| 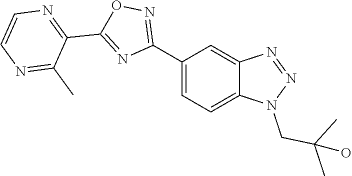 [SMILES: Cc1nccnc1-c1nc(-c2ccc3c(c2)nnn3CC(C)(C)O)no1] | 668 | 2-methyl-1-(5-(5-(3- methylpyrazin-2-yl)- 1,2,4-oxadiazol-3-yl)- 1H-benzo[d][1,2,3] triazol-1-yl)propan-2-ol | 351.14 | 352.2 |
| 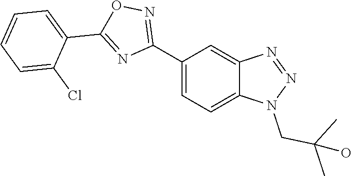 [SMILES: CC(C)(O)Cn1nnc2cc(-c3noc(-c4ccccc4Cl)n3)ccc21] | 669 | 1-(5-(5-(2-chlorophenyl)- 1,2,4-oxadiazol-3-yl)- 1H-benzo[d][1,2,3] triazol-1-yl)-2- methylpropan-2-ol | 369.1 | 369.9 |
| 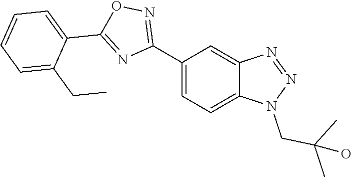 [SMILES: CCc1ccccc1-c1nc(-c2ccc3c(c2)nnn3CC(C)(C)O)no1] | 670 | 1-(5-(5-(2-ethylphenyl)- 1,2,4-oxadiazol-3-yl)- 1H-benzo[d][1,2,3] triazol-1-yl)-2- methylpropan-2-ol | 363.17 | 364 |
| 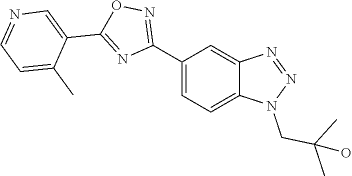 [SMILES: Cc1ccncc1-c1nc(-c2ccc3c(c2)nnn3CC(C)(C)O)no1] | 671 | 2-methyl-1-(5-(5-(4- methylpyridin-3- yl)-1,2,4-oxadiazol- 3-yl)-1H-benzo[d] [1,2,3]triazol-1- yl)propan-2-ol | 350.15 | 351.2 |
| 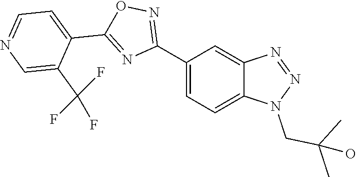 [SMILES: CC(C)(O)Cn1nnc2cc(-c3noc(-c4ccncc4C(F)(F)F)n3)ccc21] | 672 | 2-methyl-1-(5-(5-(3- (trifluoromethyl)pyridin- 4-yl)-1,2,4-oxadiazol- 3-yl)-1H-benzo[d] [1,2,3]triazol-1- yl)propan-2-ol | 404.12 | 405.2 |
| 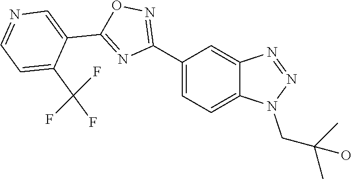 [SMILES: CC(C)(O)Cn1nnc2cc(-c3noc(-c4cnccc4C(F)(F)F)n3)ccc21] | 673 | 2-methyl-1-(5-(5-(4- (trifluoromethyl)pyridin- 3-yl)-1,2,4-oxadiazol- 3-yl)-1H-benzo[d] [1,2,3]triazol-1- yl)propan-2-ol | 404.12 | 405.2 |
| 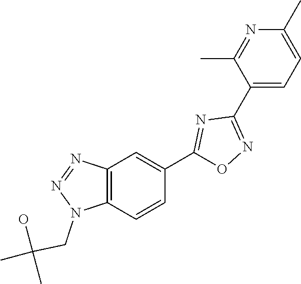 [SMILES: Cc1ccc(-c2noc(-c3ccc4c(c3)nnn4CC(C)(C)O)n2)c(C)n1] | 675 | 1-{5-[3-(2,6-dimethyl- pyridin-3-yl)-1,2,4- oxadiazol-5-yl]-1H- 1,2,3-benzotriazol-1- yl}-2-methylpropan-2-ol | 364.16 | 365.1 |
| 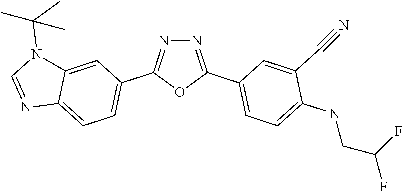 [SMILES: CC(C)(C)n1cnc2ccc(-c3nnc(-c4ccc(NCC(F)F)c(C#N)c4)o3)cc21] | 676 | 5-[5-(1-tert-butyl-1H- 1,3-benzodiazol-6- yl)-1,3,4-oxadiazol- 2-yl]-2-[(2,2- difluoroethyl)amino] benzonitrile | 422.17 | 423.1 |
| 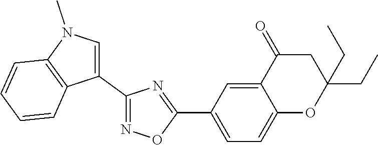 [SMILES: CCC1(CC)CC(=O)c2cc(-c3nc(-c4cn(C)c5ccccc45)no3)ccc2O1] | 677 | 2,2-diethyl-6-[3-(1- methyl-1H-indol-3- yl)-1,2,4-oxadiazol-5- yl]-3,4-dihydro-2H- 1-benzopyran-4-one | 401.17 | 402.1 |
| 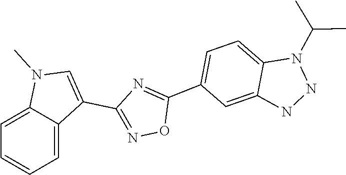 [SMILES: CC(C)N1NNc2cc(-c3nc(-c4cn(C)c5ccccc45)no3)ccc21] | 678 | 5-[3-(1-methyl-1H- indol-3-yl)-1,2,4- oxadiazol-5-yl]-1- (propan-2-yl)-1H- 1,2,3-benzotriazole | 358.15 | 359.1 |
| 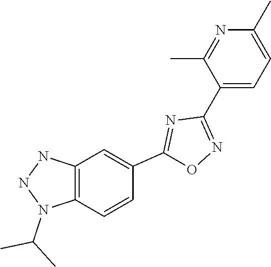 [SMILES: Cc1ccc(-c2noc(-c3ccc4c(c3)NNN4C(C)C)n2)c(C)n1] | 679 | 5-[3-(2,6-dimethyl- pyridin-3-yl)-1,2,4- oxadiazol-5-yl]-1- (propan-2-yl)-1H- 1,2,3-benzotriazole | 334.15 | 335.1 |
| 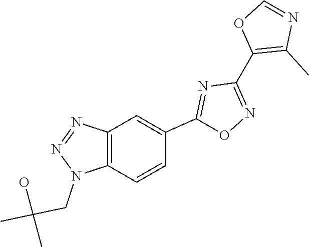 [SMILES: Cc1ncoc1-c1noc(-c2ccc3c(c2)nnn3CC(C)(C)O)n1] | 680 | 2-methyl-1-{5-[3-(4- methyl-1,3-oxazol- 5-yl)-1,2,4-oxadiazol- 5-yl]-1H-1,2,3-benzo- triazol-1-yl}propan- 2-ol | 340.13 | 341.1 |
| 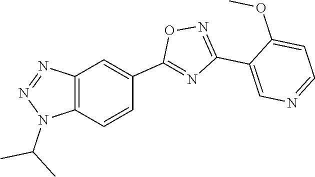 [SMILES: COc1ccncc1-c1noc(-c2ccc3c(c2)nnn3C(C)C)n1] | 681 | 5-(1-isopropyl-1H- benzo[d][1,2,3]triazol- 5-yl)-3-(4-methoxy- pyridin-3-yl)-1,2,4- oxadiazole | 336.13 | 337.3 |
| 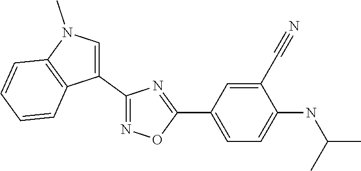 [SMILES: CC(C)Nc1ccc(-c2nc(-c3cn(C)c4ccccc34)no2)cc1C#N] | 682 | 5-[3-(1-methyl-1H- indol-3-yl)-1,2,4- oxadiazol-5-yl]-2- [(propan-2- yl)amino]benzonitrile | 357.16 | 358.2 |
| 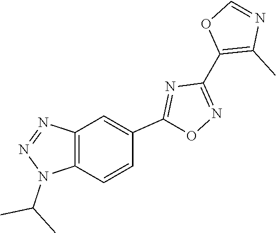 [SMILES: Cc1ncoc1-c1noc(-c2ccc3c(c2)nnn3C(C)C)n1] | 683 | 5-[3-(4-methyl-1,3- oxazol-5-yl)-1,2,4- oxadiazol-5-yl]-1- (propan-2-yl)-1H- 1,2,3-benzotriazole | 310.12 | 311.1 |
| 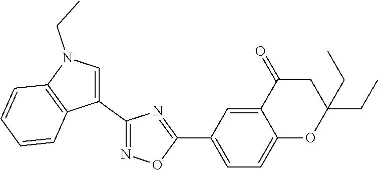 [SMILES: CCn1cc(-c2noc(-c3ccc4c(c3)C(=O)CC(CC)(CC)O4)n2)c2ccccc21] | 684 | 2,2-diethyl-6-[3-(1- ethyl-1H-indol- 3-yl)-1,2,4-oxadiazol- 5-yl]-3,4-dihydro-2H-1- benzopyran-4-one | 415.19 | 416.2 |
| 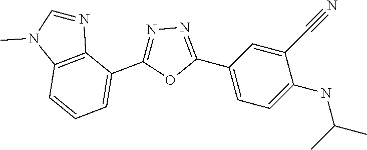 [SMILES: CC(C)Nc1ccc(-c2nnc(-c3cccc4c3ncn4C)o2)cc1C#N] | 685 | 5-[5-(1-methyl-1H- 1,3-benzodiazol-4-yl)- 1,3,4-oxadiazol- 2-yl]-2-[(propan-2- yl)amino]benzonitrile | 358.15 | 359.2 |
| 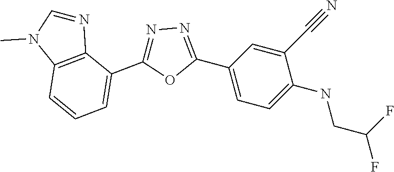 [SMILES: Cn1cnc2c(-c3nnc(-c4ccc(NCC(F)F)c(C#N)c4)o3)cccc21] | 686 | 2-[(2,2-difluoro- ethyl)aminol-5-[5-(1- methyl-1H-1,3- benzodiazol-4-yl)- 1,3,4-oxadiazol-2- yl]benzonitrile | 380.12 | 381.2 |
| 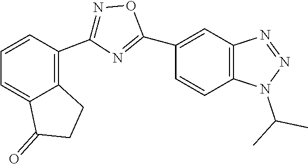 [SMILES: CC(C)n1nnc2cc(-c3nc(-c4cccc5c4CCC5=O)no3)ccc21] | 687 | 4-{5-[1-(propan-2-yl)- 1H-1,2,3-benzotriazol-5- yl]-1,2,4-oxadiazol-3-yl}- 2,3-dihydro-1H-inden- 1-one | 359.14 | 360 |
| 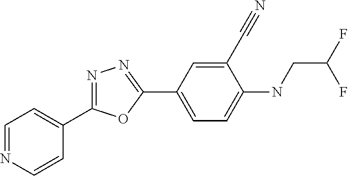 [SMILES: N#Cc1cc(-c2nnc(-c3ccncc3)o2)ccc1NCC(F)F] | 688 | 2-((2,2-difluoro- ethyl)amino)-5-(5- (pyridin-4-yl)-1,3,4- oxadiazol-2- yl)benzonitrile | 327.09 | 328.3 |
| 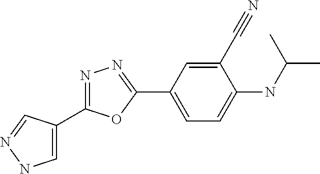 [SMILES: CC(C)Nc1ccc(-c2nnc(-c3cn[nH]c3)o2)cc1C#N] | 689 | 5-(5-(1H-pyrazol-4- yl)-1,3,4-oxadiazol- 2-yl)-2-(isopropyl- amino)benzonitrile | 294.12 | 295.3 |
| 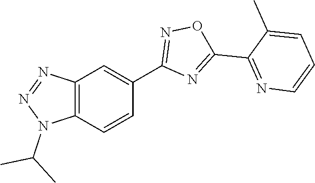 [SMILES: Cc1cccnc1-c1nc(-c2ccc3c(c2)nnn3C(C)C)no1] | 690 | 3-(1-isopropyl-1H- benzo[d][1,2,3]triazol- 5-yl)-5-(3-methyl- pyridin-2-yl)-1,2,4- oxadiazole | 320.14 | 321.3 |
| 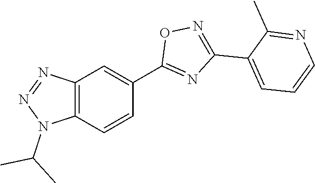 [SMILES: Cc1ncccc1-c1noc(-c2ccc3c(c2)nnn3C(C)C)n1] | 691 | 5-(1-isopropyl-1H- benzo[d][1,2,3]triazol- 5-yl)-3-(2-methyl- pyridin-3-yl)-1,2,4- oxadiazole | 320.14 | 321.2 321.2 |
| 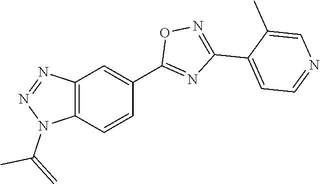 [SMILES: C=C(C)n1nnc2cc(-c3nc(-c4ccncc4C)no3)ccc21] | 692 | 5-(1-isopropyl-1H- benzo[d][1,2,3]triazol- 5-yl)-3-(3-methyl- pyridin-4-yl)-1,2,4- oxadiazole | 320.14 | 321.2 |
| 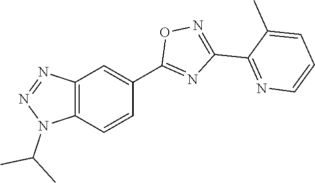 [SMILES: Cc1cccnc1-c1noc(-c2ccc3c(c2)nnn3C(C)C)n1] | 693 | 5-(1-isopropyl-1H- benzo[d][1,2,3]triazol-5- yl)-3-(3-methylpyridin- 2-yl)-1,2,4-oxadiazole | 320.14 | 321.1 |
| 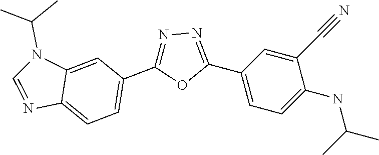 [SMILES: CC(C)Nc1ccc(-c2nnc(-c3ccc4ncn(C(C)C)c4c3)o2)cc1C#N] | 694 | 5-{5-[1-(propan-2-yl)- 1H-1,3-benzodiazol-6- yl]-1,3,4-oxadiazol- 2-yl}-2-[(propan-2- yl)amino]benzonitrile | 386.19 | 387.2 |
| 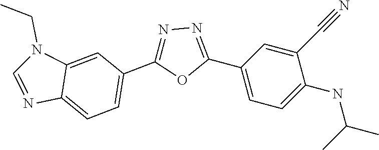 [SMILES: CCn1cnc2ccc(-c3nnc(-c4ccc(NC(C)C)c(C#N)c4)o3)cc21] | 695 | 5-[5-(1-ethyl-1H-1,3- benzodiazol-6-yl)-1,3,4- oxadiazol-2-yl]-2- [(propan-2- yl)amino]benzonitrile | 372.17 | 373.1 |
| 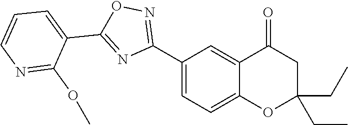 [SMILES: CCC1(CC)CC(=O)c2cc(-c3noc(-c4cccnc4OC)n3)ccc2O1] | 696 | 2,2-diethyl-6-[5- (2-methoxypyridin- 3-yl)-1,2,4-oxadiazol- 3-yl]-3,4-dihydro-2H- 1-benzopyran-4-one | 379.15 | 380.2 |
| 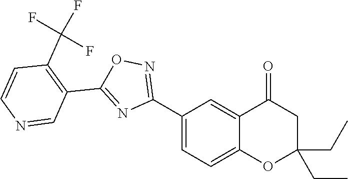 [SMILES: CCC1(CC)CC(=O)c2cc(-c3noc(-c4cnccc4C(F)(F)F)n3)ccc2O1] | 697 | 2,2-diethyl-6-{5-[4- (trifluoromethyl)pyridin- 3-yl]-1,2,4-oxadiazol- 3-yl}-3,4-dihydro- 2H-1-benzopyran- 4-one | 417.13 | 418.2 |
| 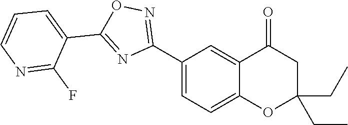 [SMILES: CCC1(CC)CC(=O)c2cc(-c3noc(-c4cccnc4F)n3)ccc2O1] | 698 | 2,2-diethyl-6-[5-(2- fluoropyridin-3- yl)-1,2,4-oxadiazol- 3-yl]-3,4-dihydro- 2H-1-benzopyran- 4-one | 367.13 | 368.1 |
| 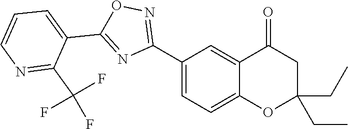 [SMILES: CCC1(CC)CC(=O)c2cc(-c3noc(-c4cccnc4C(F)(F)F)n3)ccc2O1] | 699 | 2,2-diethyl-6-{5-[2- (trifluoromethyl)pyridin- 3-yl]-1,2,4-oxadiazol- 3-yl}-3,4-dihydro-2H- 1-benzopyran-4-one | 417.13 | 418.1 |
| 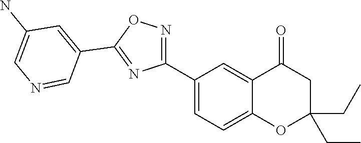 [SMILES: CCC1(CC)CC(=O)c2cc(-c3noc(-c4cncc(N)c4)n3)ccc2O1] | 700 | 6-[5-(5-aminopyridin-3- yl)-1,2,4-oxadiazol- 3-yl]-2,2-diethyl-3,4- dihydro-2H-1- benzopyran-4-one | 364.15 | 365.1 |
| 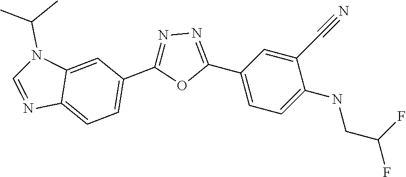 [SMILES: CC(C)n1cnc2ccc(-c3nnc(-c4ccc(NCC(F)F)c(C#N)c4)o3)cc21] | 701 | 2-[(2,2-difluoro- ethyl)amino]-5-{5- [1-(propan-2-yl)-1H- 1,3-benzodiazol-6- yl]-1,3,4-oxadiazol- 2-yl}benzonitrile | 408.15 | 409.1 |
| 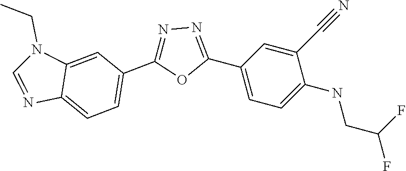 [SMILES: CCn1cnc2ccc(-c3nnc(-c4ccc(NCC(F)F)c(C#N)c4)o3)cc21] | 702 | 2-[(2,2-difluoro- ethyl)amino]-5-[5- (1-ethyl-1H-1,3- benzodiazol-6-yl)- 1,3,4-oxadiazol-2- yl]benzonitrile | 394.14 | 395.1 |
| 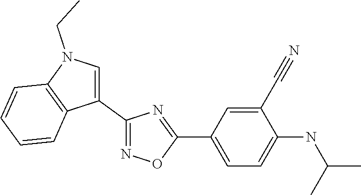 [SMILES: CCn1cc(-c2noc(-c3ccc(NC(C)C)c(C#N)c3)n2)c2ccccc21] | 703 | 5-[3-(1-ethyl-1H-indol- 3-yl)-1,2,4-oxadiazol- 5-yl]-2-[(propan-2- yl)amino]benzonitrile | 371.17 | 372.1 |
| 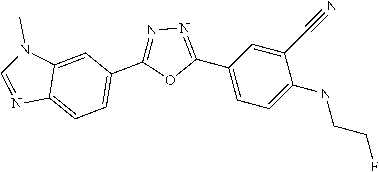 [SMILES: Cn1cnc2ccc(-c3nnc(-c4ccc(NCCF)c(C#N)c4)o3)cc21] | 704 | 2-[(2-fluoroethyl)amino]- 5-[5-(1-methyl-1H- 1,3-benzodiazol-6-yl)- 1,3,4-oxadiazol-2- yl]benzonitrile | 362.13 | 363.1 |
| 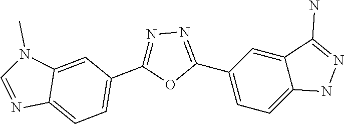 [SMILES: Cn1cnc2ccc(-c3nnc(-c4ccc5[nH]nc(N)c5c4)o3)cc21] | 705 | 5-[5-(1-methyl-1H-1,3- benzodiazol-6-yl)- 1,3,4-oxadiazol-2-yl]- 1H-indazol-3-amine | 331.12 | 332 |
| 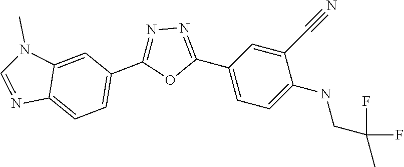 [SMILES: Cn1cnc2ccc(-c3nnc(-c4ccc(NCC(C)(F)F)c(C#N)c4)o3)cc21] | 706 | 2-[(2,2-difluoro- propyl)amino]-5-[5- (1-methyl-1H-1,3- benzodiazol-6-yl)- 1,3,4-oxadiazol-2- yl]benzonitrile | 394.14 | 395.1 |
| 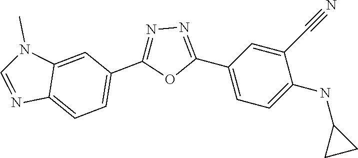 [SMILES: Cn1cnc2ccc(-c3nnc(-c4ccc(NC5CC5)c(C#N)c4)o3)cc21] | 707 | 2-(cyclopropylamino)- 5-[5-(1-methyl-1H- 1,3-benzodiazol-6-yl)- 1,3,4-oxadiazol-2- yl]benzonitrile | 356.14 | 357.1 |
| 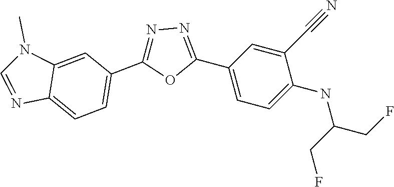 [SMILES: Cn1cnc2ccc(-c3nnc(-c4ccc(NC(CF)CF)c(C#N)c4)o3)cc21] | 708 | 2-[(1,3-difluoro- propan-2-yl)amino]- 5-[5-(1-methyl-1H-1,3- benzodiazol-6-yl)- 1,3,4-oxadiazol- 2- yl]benzonitrile | 394.14 | 395.1 |
| 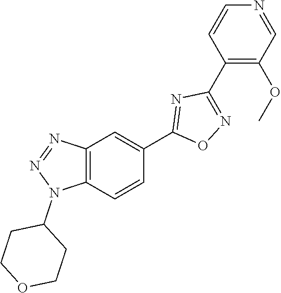 [SMILES: COc1cnccc1-c1noc(-c2ccc3c(c2)nnn3C2CCOCC2)n1] | 709 | 5-[3-(3-methoxy- pyridin-4-yl)-1,2,4- oxadiazol-5-yl]- 1-(oxan-4-yl)-1H- 1,2,3-benzotriazole | 378.14 | 379.1 |
| 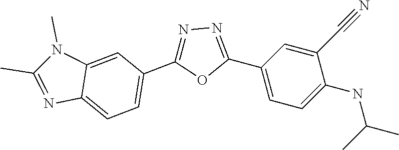 [SMILES: Cc1nc2ccc(-c3nnc(-c4ccc(NC(C)C)c(C#N)c4)o3)cc2n1C] | 710 | 5-[5-(1,2-dimethyl-1H- 1,3-benzodiazol-6- yl)-1,3,4-oxadiazol- 2-yl]-2-[(propan-2- yl)amino]benzonitrile | 372.17 | 373.1 |
| 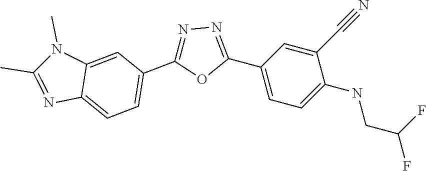 [SMILES: Cc1nc2ccc(-c3nnc(-c4ccc(NCC(F)F)c(C#N)c4)o3)cc2n1C] | 711 | 2-[(2,2-difluoro- ethyl)amino]-5-[5- (1,2-dimethyl-1H- 1,3-benzodiazol-6- yl)-1,3,4-oxadiazol- 2-yl]benzonitrile | 394.14 | 395.1 |
| 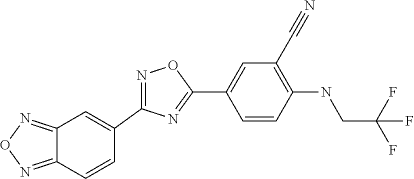 [SMILES: N#Cc1cc(-c2nc(-c3ccc4nonc4c3)no2)ccc1NCC(F)(F)F] | 712 | 5-[3-(2,1,3-benzoxa- diazol-5-yl)-1,2,4- oxadiazol-5-yl]-2- [(2,2,2-trifluoroethyl) amino]benzonitrile | 386.07 | 387 |
| 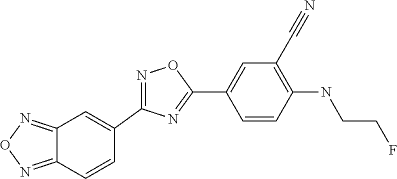 [SMILES: N#Cc1cc(-c2nc(-c3ccc4nonc4c3)no2)ccc1NCCF] | 713 | 5-[3-(2,1,3-benzoxa- diazol-5-yl)-1,2,4- oxadiazol-5-yl]-2- [(2-fluoroethyl) amino]benzonitrile | 350.09 | 351 |
| 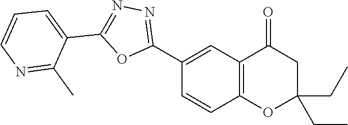 [SMILES: CCC1(CC)CC(=O)c2cc(-c3nnc(-c4cccnc4C)o3)ccc2O1] | 718 | 2,2-diethyl-6-(5-(2- methylpyridin- 3-yl)-1,3,4-oxadiazol-2- yl)chroman-4-one | 363.16 | 364.4 |
| 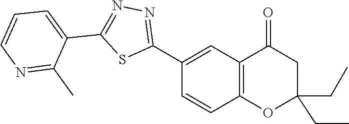 [SMILES: CCC1(CC)CC(=O)c2cc(-c3nnc(-c4cccnc4C)s3)ccc2O1] | 719 | 2,2-diethyl-6-(5-(2- methylpyridin-3- yl)-1,3,4-thiadiazol-2- yl)chroman-4-one | 379.14 | 380.2 |
| 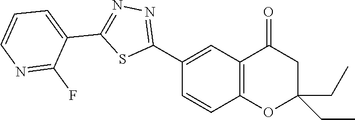 [SMILES: CCC1(CC)CC(=O)c2cc(-c3nnc(-c4cccnc4F)s3)ccc2O1] | 720 | 2,2-diethyl-6-(5-(2- fluoropyridin-3-yl)- 1,3,4-thiadiazol-2- yl)chroman-4-one | 383.11 | 384.2 |
| 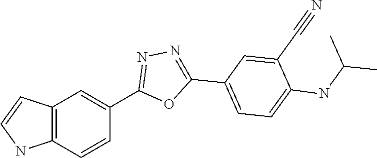 [SMILES: CC(C)Nc1ccc(-c2nnc(-c3ccc4[nH]ccc4c3)o2)cc1C#N] | 721 | 5-(5-(1H-indol-5-yl)- 1,3,4-oxadiazol-2- yl)-2-(isopropyl- amino)benzonitrile | 343.14 | 344.4 |
| 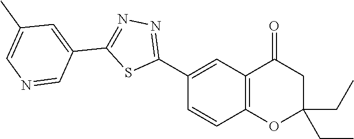 [SMILES: CCC1(CC)CC(=O)c2cc(-c3nnc(-c4cncc(C)c4)s3)ccc2O1] | 724 | 2,2-diethyl-6-(5-(5- methylpyridin-3- yl)-1,3,4-thiadiazol-2- yl)chroman-4-one | 379.14 | 380.3 |
| 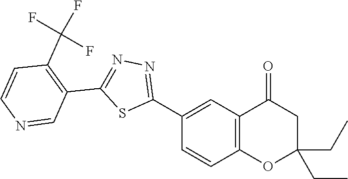 [SMILES: CCC1(CC)CC(=O)c2cc(-c3nnc(-c4cnccc4C(F)(F)F)s3)ccc2O1] | 725 | 2,2-diethyl-6-(5-(4- (trifluoromethyl)pyridin- 3-yl)-1,3,4- thiadiazol-2-yl)chroman- 4-one | 433.11 | 434.3 |
| 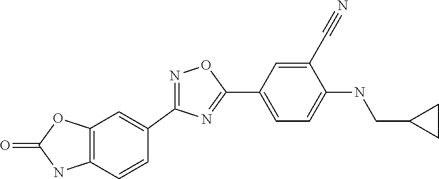 [SMILES: N#Cc1cc(-c2nc(-c3ccc4[nH]c(=O)oc4c3)no2)ccc1NCC1CC1] | 726 | 2-(cyclopropylmethyl- amino)-5-[3-(2-oxo- 3H-1,3-benzoxazol-6- yl)-1,2,4-oxadiazol- 5-yl]benzonitrile | 373.12 | 374.1 |
| 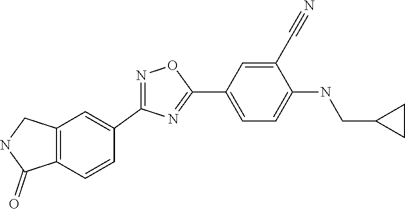 [SMILES: N#Cc1cc(-c2nc(-c3ccc4c(c3)CNC4=O)no2)ccc1NCC1CC1] | 727 | 2-((cyclopropyl- methyl)amino)-5- (3-(1-oxoisoindolin- 5-yl)-1,2,4-oxadiazol- 5-yl)benzonitrile | 371.14 | 372.1 |
| 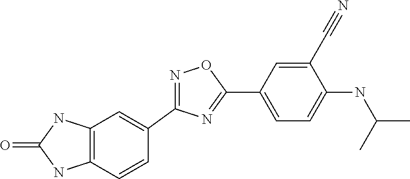 [SMILES: CC(C)Nc1ccc(-c2nc(-c3ccc4[nH]c(=O)[nH]c4c3)no2)cc1C#N] | 728 | 2-(isopropylamino)-5- (3-(2-oxo-2,3-dihydro- 1H-benzo[d]imidazol- 5-yl)-1,2,4-oxadiazol-5- yl)benzonitrile | 360.13 | 361.1 |
| 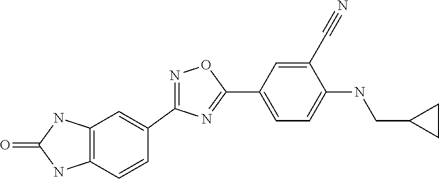 [SMILES: N#Cc1cc(-c2nc(-c3ccc4[nH]c(=O)[nH]c4c3)no2)ccc1NCC1CC1] | 729 | 2-((cyclopropyl- methyl)amino)-5-(3- (2-oxo-2,3-dihydro-1H- benzo[d]imidazol-5- yl)-1,2,4-oxadiazol- 5-yl)benzonitrile | 372.13 | 373.1 |
| 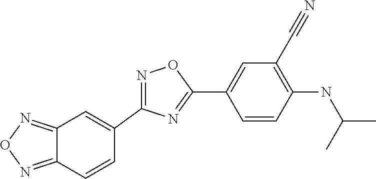 [SMILES: CC(C)Nc1ccc(-c2nc(-c3ccc4nonc4c3)no2)cc1C#N] | 730 | 5-[3-(2,1,3-benzoxa- diazol-5-yl)-1,2,4- oxadiazol-5-yl]-2- [(propan-2- yl)amino]benzonitrile | 346.12 | 347.1 |
| 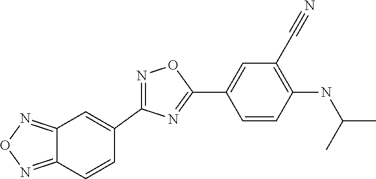 [SMILES: CC(C)Nc1ccc(-c2nc(-c3ccc4nonc4c3)no2)cc1C#N] | 730 | 5-[3-(2,1,3-benzoxa- diazol-5-yl)-1,2,4- oxadiazol-5-yl]-2- [(propan-2- yl)amino]benzonitrile | 346.12 | 347.1 |
| 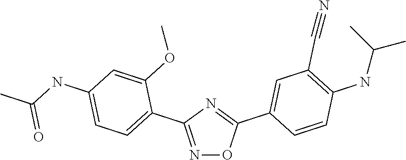 [SMILES: COc1cc(NC(C)=O)ccc1-c1noc(-c2ccc(NC(C)C)c(C#N)c2)n1] | 731 | N-[4-(5-{3-cyano-4- [(propan-2-yl)amino] phenyl}-1,2,4-oxadiazol- 3-yl)-3-methoxy phenyl]acetamide | 391.16 | 392.1 |
| 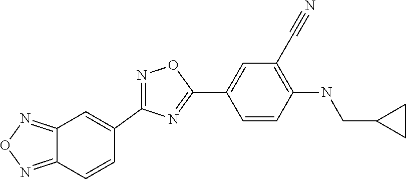 [SMILES: N#Cc1cc(-c2nc(-c3ccc4nonc4c3)no2)ccc1NCC1CC1] | 732 | 5-[3-(2,1,3-benzoxa- diazol-5-yl)- 1,2,4-oxadiazol-5- yl]-2-[(2,2-difluoro- ethyl)amino]benzonitrile | 358.12 | 369 |
| 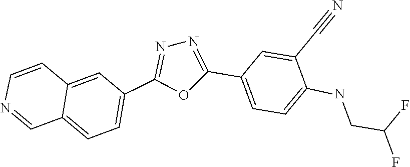 [SMILES: N#Cc1cc(-c2nnc(-c3ccc4cnccc4c3)o2)ccc1NCC(F)F] | 733 | 2-[(2,2-difluoro- ethyl)amino]-5-[5- (isoquinolin-6-yl)- 1,3,4-oxadiazol- 2-yl]benzonitrile | 377.11 | 378.1 |
| 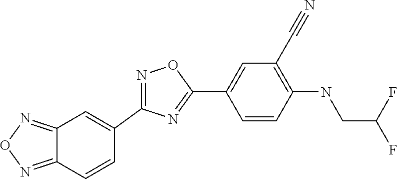 [SMILES: N#Cc1cc(-c2nc(-c3ccc4nonc4c3)no2)ccc1NCC(F)F] | 734 | 5-[3-(2,1,3-benzoxa- diazol-5-yl)-1,2,4- oxadiazol-5-yl]-2- [(cyclopropylmethyl) amino]benzonitrile | 368.08 | 359 |
| 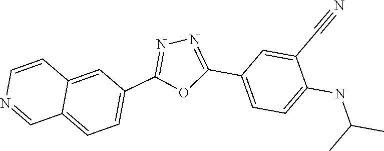 [SMILES: CC(C)Nc1ccc(-c2nnc(-c3ccc4cnccc4c3)o2)cc1C#N] | 735 | 5-[5-(isoquinolin-6-yl)- 1,3,4-oxadiazol-2-yl]- 2-[(propan-2- yl)amino]benzonitrile | 355.14 | 356.1 |
| 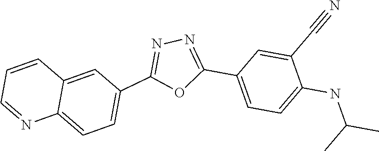 [SMILES: CC(C)Nc1ccc(-c2nnc(-c3ccc4ncccc4c3)o2)cc1C#N] | 736 | 2-[(propan-2-yl)amino]- 5-[5-(quinolin-6-yl)- 1,3,4-oxadiazol-2- yl]benzonitrile | 355.14 | 356.1 |
| 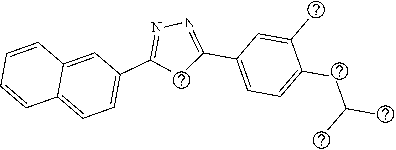 [SMILES: Cc1cc(C2=NN=C(c3ccc4ccccc4c3)C2)ccc1CC(C)C] | 736 | 2-[(propan-2-yl)amino]- 5-[5-(quinolin-6-yl)- 1,3,4-oxadiazol-2- yl]benzonitrile | 355.14 | 356.1 |
| 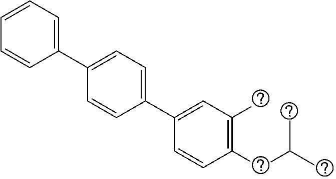 [SMILES: Cc1cc(-c2ccc(-c3ccccc3)cc2)ccc1CC(C)C] | 737 | 2-(isopropylamino)-5- (5-(pyridin-3-yl)pyrimidin- 2-yl)benzonitrile | 315.15 | 316 |
| 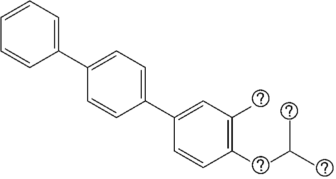 [SMILES: Cc1cc(-c2ccc(-c3ccccc3)cc2)ccc1CC(C)C] | 738 | 2-isopropoxy-5-(5- (pyridin-3-yl)pyrimidin- 2-yl)benzonitrile | 316.13 | 317 |
| 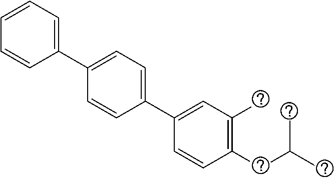 [SMILES: Cc1cc(-c2ccc(-c3ccccc3)cc2)ccc1CC(C)C] | 739 | 2-(isopropylamino)-5- (5-(pyridin-4-yl) pyrimidin-2- yl)benzonitrile | 315.15 | 316 |
| 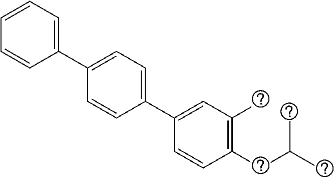 [SMILES: Cc1cc(-c2ccc(-c3ccccc3)cc2)ccc1CC(C)C] | 740 | 2-isopropoxy-5-(5- (pyridin-4-yl)pyrimidin- 2-yl)benzonitrile | 316.13 | 317 |
| 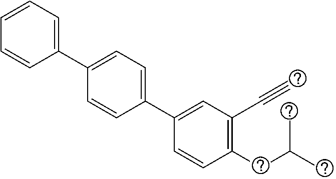 [SMILES: C#Cc1cc(-c2ccc(-c3ccccc3)cc2)ccc1CC(C)C] | 741 | 2-(isopropylamino)-5- (5-(pyridin- 2-yl)pyrimidin-2- yl)benzonitrile | 315.15 | 316 |
| 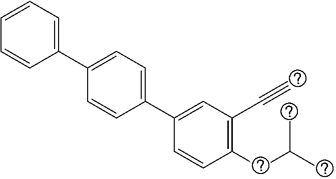 [SMILES: C#Cc1cc(-c2ccc(-c3ccccc3)cc2)ccc1CC(C)C] | 742 | 2-isopropoxy-5-(5- (pyridin-2- yl)pyrimidin-2- yl)benzonitrile | 316.15 | 317 |
| 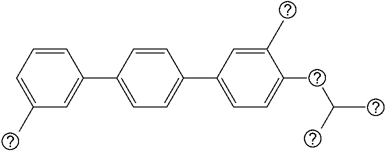 [SMILES: Cc1cccc(-c2ccc(-c3ccc(CC(C)C)c(C)c3)cc2)c1] | 743 | 5-(5-(2-hydroxy- pyridin-4- yl)pyrimidin-2-yl)- 2-(isopropyl- amino)benzonitrile | 331.14 | 332 |
| 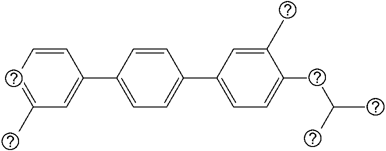 [SMILES: Cc1cccc(-c2ccc(-c3ccc(CC(C)C)c(C)c3)cc2)c1] | 744 | 5-(5-(2-hydroxy- pyridin-4- yl)pyrimidin-2-yl)- 2-isopropoxy- benzonitrile | 332.13 | 333 |
| 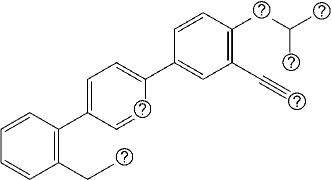 [SMILES: C#Cc1cc(-c2ccc(-c3ccccc3CC)cc2)ccc1CC(C)C] | 745 | 2-isopropoxy-5-(5- (2-methoxy- phenyl)pyrimidin-2- yl)benzonitrile | 345.15 | 346 |
| 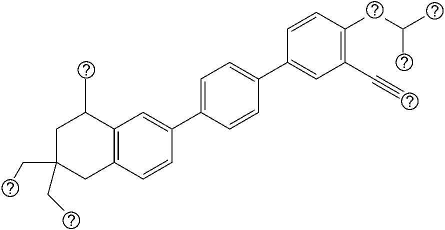 [SMILES: C#Cc1cc(-c2ccc(-c3ccc4c(c3)C(C)CC(CC)(CC)C4)cc2)ccc1CC(C)C] | 746 | 5-(5-(2,2-diethyl-4- oxochroman-6- yl)pyrimidin-2-yl)- 2-(isopropyl- amino)benzonitrile | 440.22 | 441.4 |
| 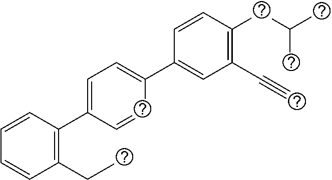 [SMILES: C#Cc1cc(-c2ccc(-c3ccccc3CC)cc2)ccc1CC(C)C] | 747 | 2-(isopropylamino)-5- (5-(2-methoxy- phenyl)pyrimidin-2- yl)benzonitrile | 344.16 | 345.3 |
| 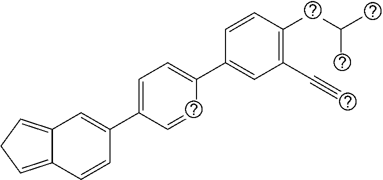 [SMILES: C#Cc1cc(-c2ccc(-c3ccc4c(c3)=CCC=4)cc2)ccc1CC(C)C] | 748 | 5-(5-(benzo[c][1,2,5] oxadiazol-5- yl)pyrimidin-2-yl)-2- isopropoxybenzonitrile | 357.12 | 357.9 |
| 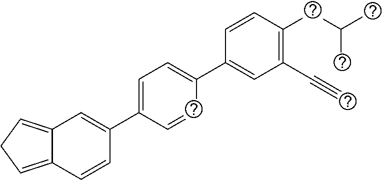 [SMILES: C#Cc1cc(-c2ccc(-c3ccc4c(c3)=CCC=4)cc2)ccc1CC(C)C] | 749 | 5-(5-(benzo[c][1,2,5] oxadiazol-5- yl)pyrimidin-2-yl)- 2-(isopropyl- amino)benzonitrile | 356.14 | 356.9 |
| 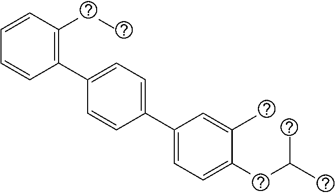 [SMILES: CCc1ccccc1-c1ccc(-c2ccc(CC(C)C)c(C)c2)cc1] | 750 | N-isopropyl-4-(5-(2- methoxyphenyl)pyrimidin- 2-yl)-2-(trifluoro- methyl)aniline | 387.16 | 388 |
| 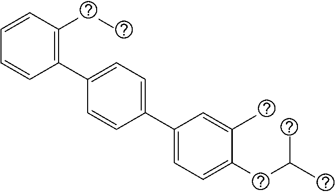 [SMILES: CCc1ccccc1-c1ccc(-c2ccc(CC(C)C)c(C)c2)cc1] | 751 | 2-(4-isopropoxy-3- (trifluoromethyl)phenyl)- 5-(2-methoxy- phenyl)pyrimidine | 388.14 | 389 |
| 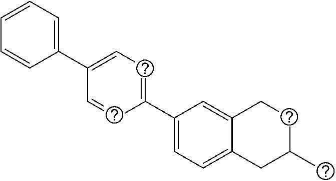 [SMILES: CC1CCc2cc(-c3ccc(-c4ccccc4)cc3)ccc2C1] | 752 | 2-(4-isopropoxy-3- (trifluoromethyl)phenyl)- 5-(pyridin-4- yl)pyrimidine | 359.13 | 360 |

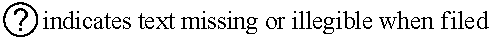

### General Procedure A:

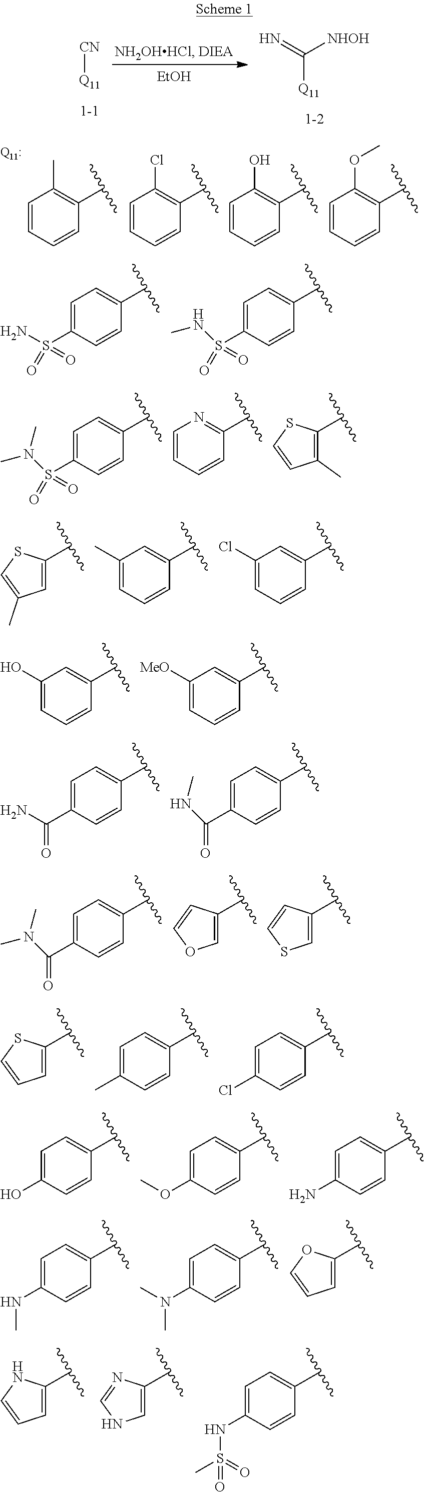

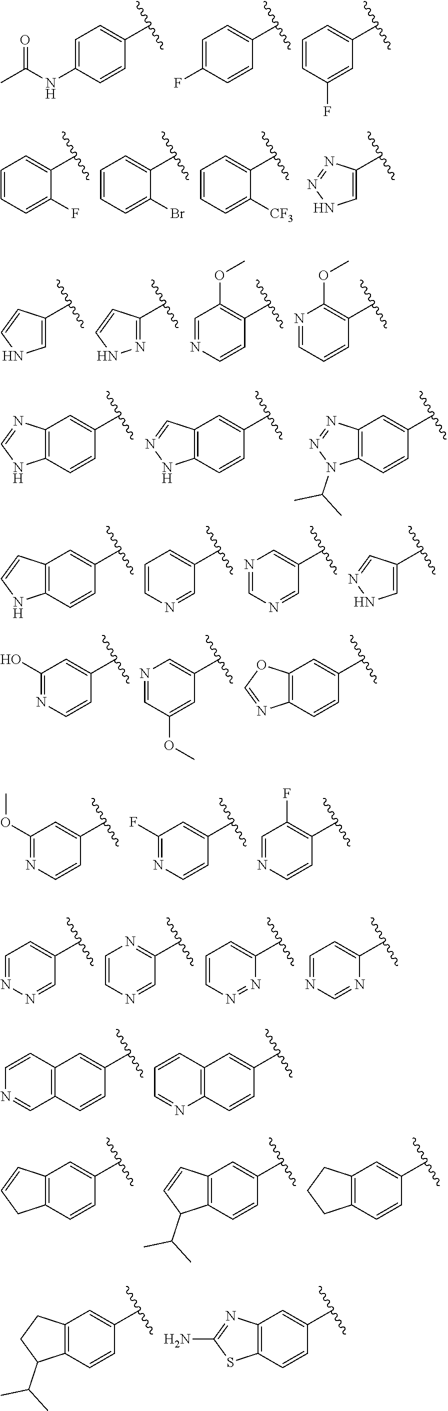

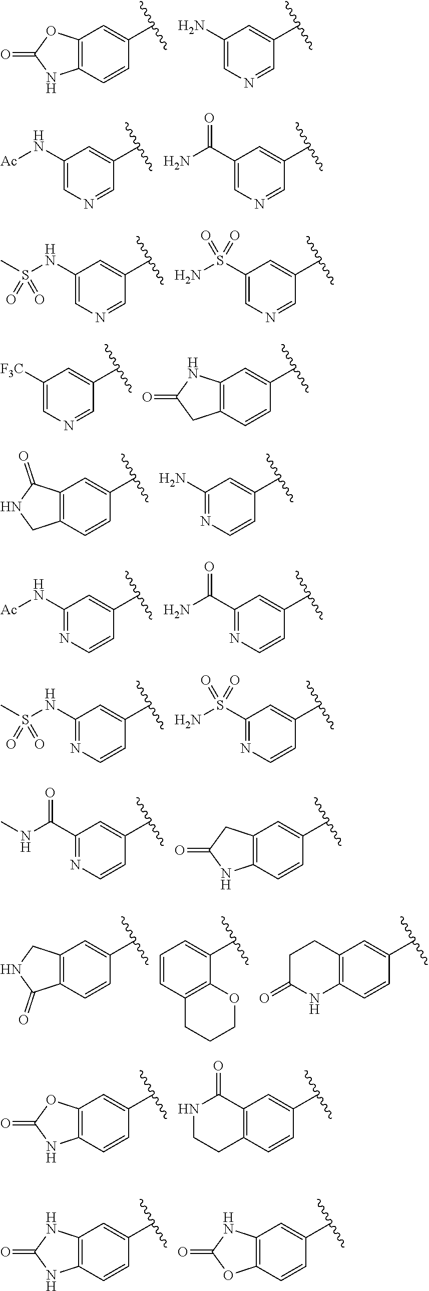

### Example 2

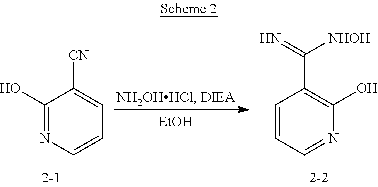

### Synthesis of Intermediate 2-2: N,2-dihydroxypyridine-3-carboximidamide

To a mixture of 2-1 (2-hydroxypyridine-3-carbonitrile) (200 mg, 1.67 mmol) in ethanol (10 mL) was added hydrochloride salt of hydroxylamine (174 mg, 2.50 mmol), diisopropylethylamine (430 mg, 3.33 mmol) at 20° C. The mixture was then heated to 90° C. and stirred for 16 hrs. The mixture was concentrated in vacuum to remove part of ethanol, the resulting mixture was filtered, and the solid was dried in vacuum which was used as the product in next step without further purification (185 mg, 69% yield).

¹H NMR (400 MHz, DMSO-d₆) δ=12.06 (br, s, 1H), 9.50 (br, s, 1H), 7.95 (dd, J=7.2, 2.4 Hz, 1H), 7.51 (dd, J=6.0, 2.0 Hz, 1H), 6.3-6.30 (m, 3H).

### General Procedure B

### Synthesis of Intermediate 3-2: 2,2-diethyl-4-oxo-3,4-dihydro-2H-1-benzopyran-6-carboxylic acid

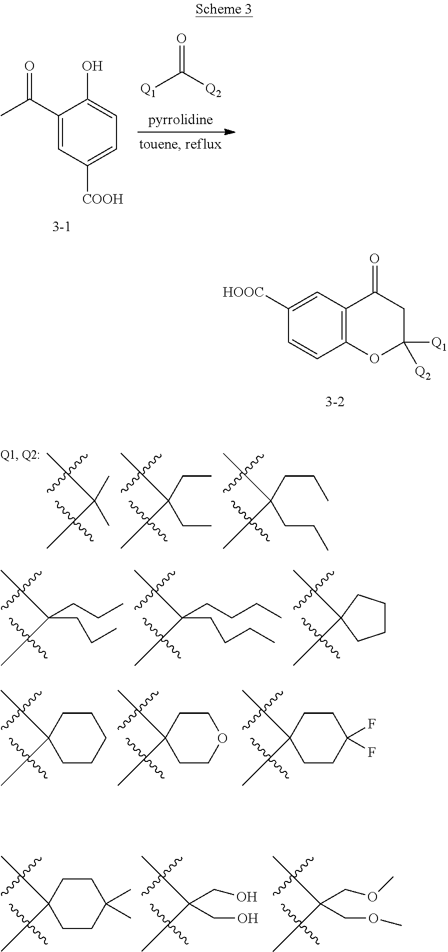

### Alternative Synthesis of Intermediate 4-4: 2,2-diethyl-4-oxo-3,4-dihydro-2H-1-benzopyran-6-carboxylic acid

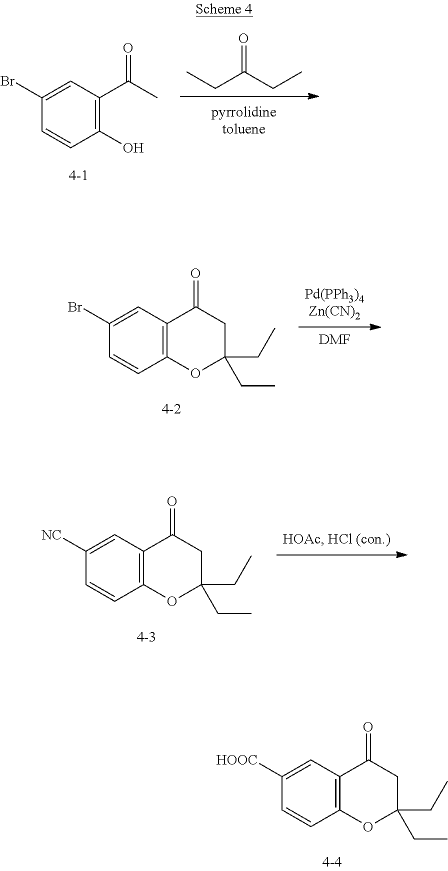

### Synthesis of 4-2: 6-bromo-2,2-diethyl-3,4-dihydro-2H-1-benzopyran-4-one

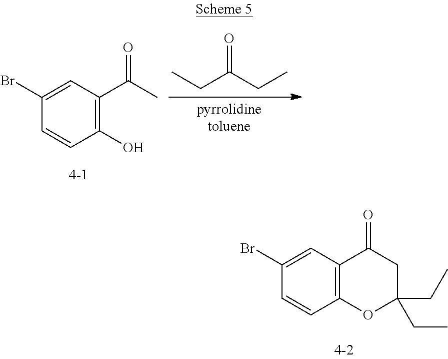

To a solution of 1-(5-bromo-2-hydroxy-phenyl)ethanone (20 g, 93.0 mmol, 1 eq) in methanol (400 mL) was added pyrrolidine (7.94 g, 112 mmol, 1.2 eq) and pentan-3-one (9.61 g, 112 mmol, 1.2 eq). The mixture was stirred at 80° C. for 16 hr. The reaction mixture diluted with water (200 mL) and extracted with ethyl acetate (200 mL×2), the combined organic layers were washed with brine (500 mL), dried over sodium sulfate, filtered and concentrated under reduced pressure to give a residue which was purified by column chromatography to give the desired product 4-2 (12 g, 46% yield) as a yellow oil.

¹H NMR (400 MHz, CDCl₃) δ=7.93 (d, J=2.5 Hz, 1H), 7.52 (dd, J=8.8, 2.6 Hz, 1H), 6.84 (d, J=8.8 Hz, 1H), 2.70 (s, 2H), 1.86-1.62 (m, 4H), 0.92 (t, J=7.5 Hz, 6H).

### Synthesis of Intermediate 4-3: 2,2-diethyl-4-oxo-3,4-dihydro-2H-1-benzopyran-6-carbonitrile

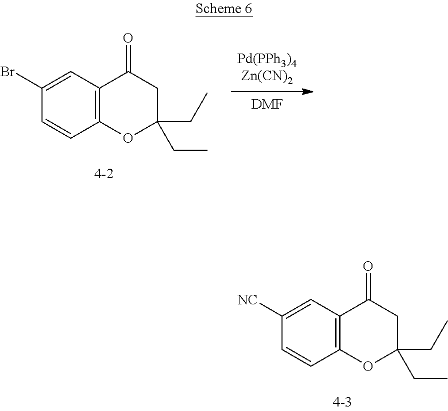

To a solution of 6-bromo-2,2-diethyl-chroman-4-one (10 g, 35.3 mmol, 1 eq) in DMF (100 mL) was added zinc cyanide (6.22 g, 53.0 mmol, 1.5 eq) and tetratriphenylphosphine palladium (4.08 g, 3.53 mmol, 0.1 eq). The mixture was stirred at 130° C. for 2 hr. The reaction mixture was diluted with water (100 mL) and extracted with ethyl acetate (100 mL×3). The combined organic layers were washed with brine (200 mL), dried over sodium sulfate, filtered and concentrated under reduced pressure to give a residue which was purified by flash silica gel chromatography (petroleum ether/ethyl acetate=100/1 to 1/1) to give the desired product 4-3 (8 g, 99% yield).

¹H NMR (400 MHz, DMSO-d₆) δ=8.08 (d, J=1.9 Hz, 1H), 7.96 (dd, J=8.7, 2.0 Hz, 1H), 7.20 (d, J=8.7 Hz, 1H), 2.88 (s, 2H), 1.86-1.59 (m, 4H), 0.86 (br, t, J=7.4 Hz, 6H).

### Scheme 7

### Synthesis of Intermediate 4-4: 2,2-diethyl-4-oxo-3,4-dihydro-2H-1-benzopyran-6-carboxylic acid

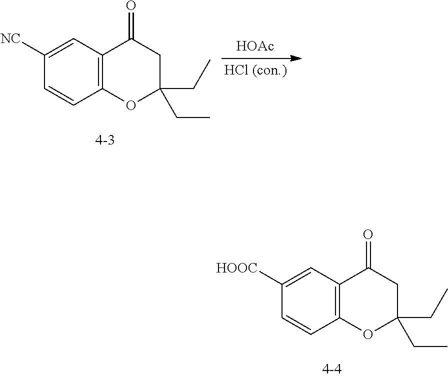 [SMILES: CCC1(CC)CC(=O)c2cc(C#N)ccc2O1 ; CCC1(CC)CC(=O)c2cc(C(=O)O)ccc2O1 ; "HOAc"]

A suspension of 2,2-diethyl-4-oxo-chromane-6-carbonitrile (6.05 g, 26.4 mmol, 1 eq) in acetic acid (60 mL) and concentrated hydrochloride solution (60 mL) was stirred at 120° C. for 16 hr, The residue was triturated with water (500 mL), filtered and dried under vacuum to give the titled product 4-4 (5.8 g, 89% yield).

¹H NMR (400 MHz, DMSO-d₆) δ=8.27 (d, J=1.9 Hz, 1H), 8.06 (dd, J=8.7, 2.0 Hz, 1H), 7.11 (d, J=8.7 Hz, 1H), 2.85 (s, 2H), 1.77-1.68 (m, 4H), 0.87 (t, J=7.4 Hz, 6H).

### General Procedure C:

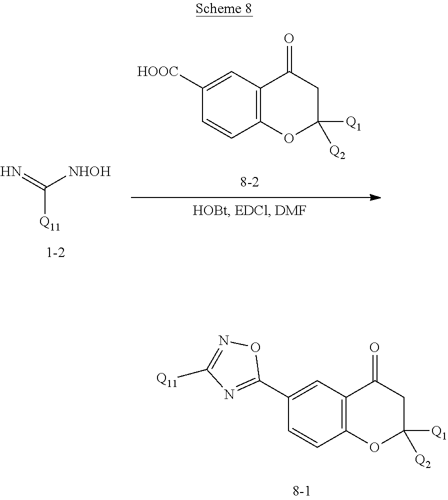

### Synthesis of Compound 9a-1: 2,2-diethyl-6-(3-(2-hydroxypyridin-3-yl)-1,2,4-oxadiazol-5-yl)chroman-4-one

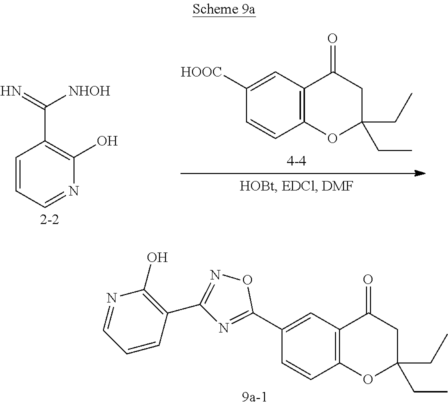

To a mixture of 4-4 (268 mg, 1.08 mmol) in N,N-dimethylformamide (6 mL) was added HOBt (159 mg, 1.18 mmol, 1.2 eq), EDCI (225 mg, 1.18 mmol, 1.2 eq) at 20° C. under nitrogen atmosphere. The mixture was stirred for 30 min, then, 2-2 (150 mg, 980 umol, 1 eq) was added, and the resultant mixture was then heated to 120° C. and stirred for 2 hrs. The mixture was diluted with water (20 mL), extracted with ethyl acetate (20 mL×3). The combined organic phase was washed by brine (50 mL), dried over sodium sulfate, concentrated in vacuum. The residue was purified by prep-HPLC (column: Phenomenex Gemini 150×25 mm×10 um; mobile phase: [water (0.05% ammonia hydroxide v/v)-ACN]; B %: 35%-65%, 12 min) to give the product 9-1 as a white solid (20 mg, 6% yield).

¹H NMR (400 MHz, DMSO-d₆) δ=12.23 (br, s, 1H), 8.44 (d, J=2.0 Hz, 1H), 8.33 (dd, J=7.2, 2.0 Hz, 1H), 8.28 (dd, J=8.8, 2.4 Hz, 1H), 7.68 (dd, J=6.0, 2.0 Hz, 1H), 7.29 (d, J=8.8 Hz, 1H), 6.42 (t, J=6.8 Hz, 1H), 2.93 (s, 2H), 1.81-1.70 (m, 4H), 0.90 (t, J=7.2 Hz, 6H).

### Synthesis of Compound 2,2-diethyl-6-[3-(1H-pyrazol-4-yl)-1,2,4-oxadiazol-5-yl]-3H-1-benzopyran-4-one

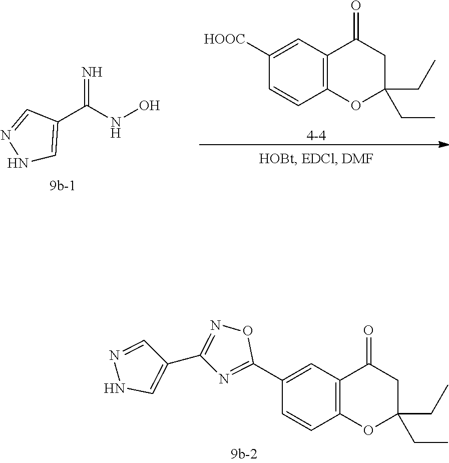 [SMILES: N=C(NO)c1cn[nH]c1 ; CCC1(CC)CC(=O)c2cc(C(=O)O)ccc2O1 ; CCC1(CC)CC(=O)c2cc(-c3nc(-c4cn[nH]c4)no3)ccc2O1]

To a solution of compound 4-4 (16.41 g, 66.08 mmol, 1 eq.) in DMF (50 mL) was added EDCI (15.20 g, 79.29 mmol, 1.2 eq.) and HOBt (8.93 g, 66.08 mmol, 1.0 eq.), stirred at 20° C. for 0.5 hour. Then compound 9b-1 (10 g, 79.29 mmol, 1.2 eq.) was added. The mixture was stirred at 20° C. for 0.5 hour, then heated to 120° C. and stirred for 2 hours. The mixture was diluted with water (100 mL), extracted with EtOAc (150 mL*3), dried with sodium sulfate, filtered and concentrated. The residue was purified by silica gel column chromatography (PE:EA=3:1) to give 9b-2 (7.2 g, yield: 30%) as white solid.

¹H NMR (400 MHz, DMSO-d₆) δ=13.49 (br. s, 1H), 8.47 (s, 1H), 8.42 (d, J=2.3 Hz, 1H), 8.26 (dd, J=2.3, 8.8 Hz, 1H), 8.06 (s, 1H), 7.26 (d, J=8.7 Hz, 1H), 2.91 (s, 2H), 1.79-1.69 (m, 4H), 0.89 (t, J=7.4 Hz, 6H).

### General Procedure D:

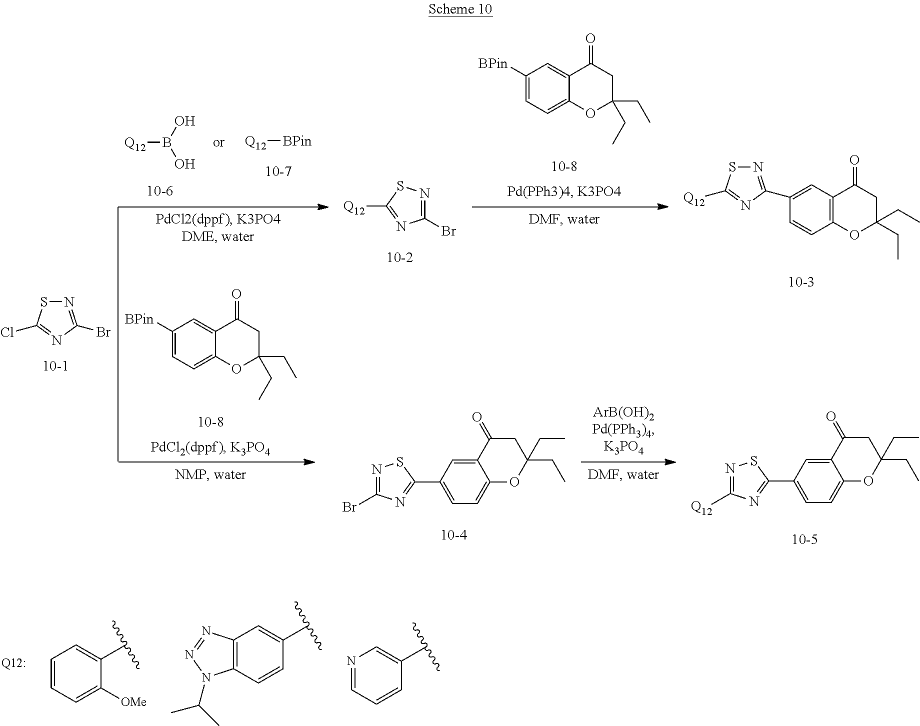

### Synthesis of 11-1: 3-bromo-5-(3-methoxyphenyl)-1,2,4-thiadiazole

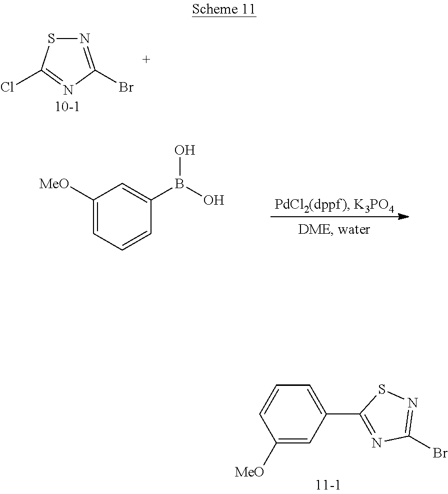 [SMILES: Clc1nc(Br)ns1 ; COc1cccc(B(O)O)c1 ; COc1cccc(-c2nc(Br)ns2)c1]

To a solution of (3-methoxyphenyl)boronic acid (247.25 mg, 1.63 mmol, 1 eq) in DME (5 mL) was added Pd(dppf)Cl₂ (119.06 mg, 162.71 umol, 0.1 eq), K₃PO₄ (1.04 g, 4.88 mmol, 3 eq) and 10-1, 3-bromo-5-chloro-1,2,4-thiadiazole (649.07 mg, 3.25 mmol, 2 eq). The mixture was stirred at 80° C. for 0.5 h. The reaction mixture was diluted with water (50 mL) and extracted with EA (50 mL×3). The combined organic layers were dried over Na₂SO₄, filtered and concentrated under reduced pressure to give a residue. The residue was purified by flash silica gel chromatography (PE/EA=10/1) to give 11-1 (3-bromo-5-(3-methoxyphenyl)-1,2,4-thiadiazole) (200 mg, 737.64 umol, 45% yield) as a white solid.

¹H NMR (400 MHz, DMSO-d₆) δ=8.23 (dd, J=1.6, 7.8 Hz, 1H), 7.78-7.57 (m, 1H), 7.38 (d, J=8.4 Hz, 1H), 7.21 (t, J=7.6 Hz, 1H), 4.13 (s, 3H).

### Synthesis of 12-1 2,2-diethyl-6-[5-(2-methoxyphenyl)-1,2,4-thiadiazol-3-yl]-3,4-dihydro-2H-1-benzopyran-4-one

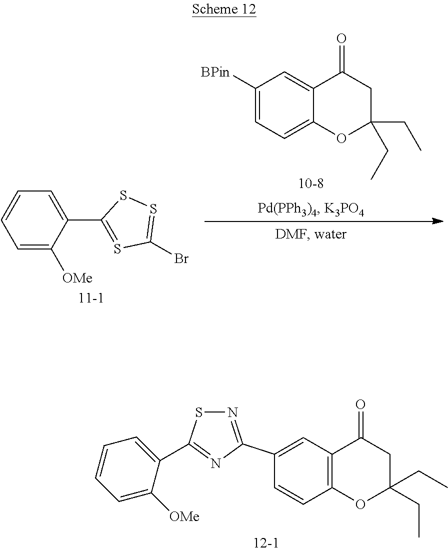

To a solution of 3-bromo-5-(2-methoxyphenyl)-1,2,4-thiadiazole (100 mg, 368.82 umol, 1 eq) and 2,2-diethyl-6-(4,4,5,5-tetramethyl-1,3,2-dioxaborolan-2-yl)chroman-4-one (146.15 mg, 442.59 umol, 1.2 eq) in DMF (1 mL) and H₂O (0.5 mL) was added K3P04 (234.87 mg, 1.11 mmol, 3 eq) and Pd(PPh₃)₄ (42.62 mg, 36.88 umol, 0.1 eq) and stirred at 120° C. for 0.25 h. The residue was purified by prep-HPLC (column: Phenomenex Synergi C18 150×25×0 um; mobile phase: [water (0.225% FA)-ACN]; B %: 70%-100%, 10 min) to give 2,2-diethyl-6-[5-(2-methoxyphenyl)-1,2,4-thiadiazol-3-yl]chroman-4-one (34.5 mg, 87.46 umol, 23.71% yield, 100% purity) as a yellow solid.

¹H NMR (400 MHz, DMSO-d₆) δ=8.66 (d, J=2.1 Hz, 1H), 8.52-8.41 (m, 2H), 7.71-7.62 (m, 1H), 7.39 (d, J=8.3 Hz, 1H), 7.26 (t, J=7.5 Hz, 1H), 7.19 (d, J=8.7 Hz, 1H), 4.14 (s, 3H), 2.88 (s, 2H), 1.76 (quint, J=7.2, 14.4 Hz, 4H), 0.90 (t, J=7.4 Hz, 6H).

### Synthesis of 10-8 2,2-diethyl-6-(tetramethyl-1,3,2-dioxaborolan-2-yl)-3,4-dihydro-2H-1-benzopyran-4-one

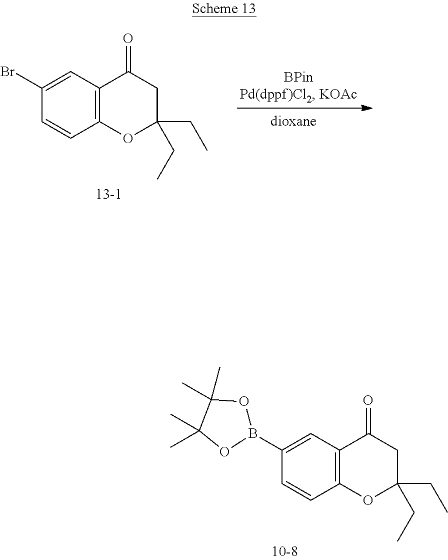 [SMILES: CCC1(CC)CC(=O)c2cc(Br)ccc2O1 ; CCC1(CC)CC(=O)c2cc(B3OC(C)(C)C(C)(C)O3)ccc2O1]

To a solution of 6-bromo-2,2-diethyl-chroman-4-one (1 g, 3.53 mmol, 1 eq) in dioxane (10 mL) was added 4,4,5,5-tetramethyl-2-(4,4,5,5-tetramethyl-1,3,2-dioxaborolan-2-yl)-1,3,2-dioxaborolane (986.48 mg, 3.88 mmol, 1.1 eq), DPPF (195.78 mg, 353.16 umol, 0.1 eq), Pd(dppf)Cl₂ (258.41 mg, 353.16 umol, 0.1 eq) and KOAc (415.92 mg, 4.24 mmol, 1.2 eq), the mixture was stirred at 100° C. for 4 h, The reaction mixture was diluted with water (100 mL) and extracted with (EA 100 mL×2). The combined organic layers were dried over Na₂SO₄, filtered and concentrated under reduced pressure to give a residue. The residue was purified by flash silica gel chromatography (PE/EA=1/1) to give 2,2-diethyl-6-(4,4,5,5-tetramethyl-1,3,2-dioxaborolan-2-yl)chroman-4-one (1 g, 3.03 mmol, 85.75% yield) as a white solid.

¹H NMR (400 MHz, CHLOROFORM-d) δ=8.26 (d, J=1.6 Hz, 1H), 7.86-7.75 (m, 1H), 6.85 (d, J=8.3 Hz, 1H), 2.64 (s, 2H), 1.82-1.58 (m, 4H), 1.32-1.21 (m, 12H), 0.85 (t, J=7.5 Hz, 6H).

### Synthesis of 14-5: 1-(propan-2-yl)-5-(tetramethyl-1,3,2-dioxaborolan-2-yl)-1H-1,2,3-benzotriazole

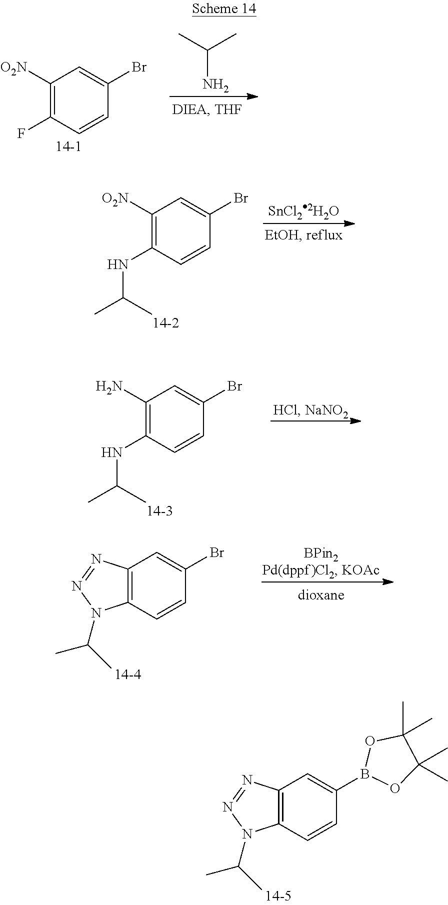

### Synthesis of 14-2 4-bromo-2-nitro-N-(propan-2-yl)aniline

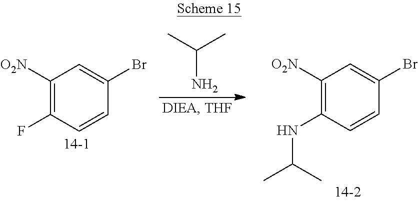

To a solution of 4-bromo-1-fluoro-2-nitro-benzene (5 g, 22.73 mmol, 2.79 mL, 1.00 eq) and propan-2-amine (2.02 g, 34.09 mmol, 2.92 mL, 1.50 eq) in THF (250.00 mL) was added DIEA (7.34 g, 56.82 mmol, 9.90 mL, 2.50 eq) at 10° C. and stirred for 1 h, The reaction mixture was diluted with water (500 mL) and extracted with EA (500 mL×2). The combined organic layers were washed with NaHCO₃ (500 mL), dried over NaSO₄, filtered and concentrated under reduced pressure to give a residue. The residue was purified by flash silica gel chromatography to give 4-bromo-N-isopropyl-2-nitro-aniline (3.2 g, 12.35 mmol, 54.34% yield) as yellow oil.

¹H NMR (400 MHz, DMSO-d₆) δ=8.14 (d, J=2.4 Hz, 1H), 7.88 (br d, J=7.5 Hz, 1H), 7.63 (dd, J=2.3, 9.3 Hz, 1H), 7.07 (d, J=9.4 Hz, 1H), 3.92 (sxtd, J=6.5, 13.2 Hz, 1H), 1.25 (d, J=6.4 Hz, 6H).

### Synthesis of 14-3 4-bromo-1-N-(propan-2-yl)benzene-1,2-diamine

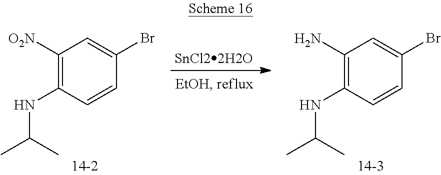

To a solution of 4-bromo-N-isopropyl-2-nitro-aniline (2 g, 7.72 mmol, 1 eq) in EtOH (20 mL) was added SnCl₂.2H₂O (5.23 g, 23.16 mmol, 1.93 mL, 3 eq) and stirred at 80° C. for 16 h. The reaction mixture was quenched with aqueous NaOH (4 M, 50 mL), and then diluted with water (50 mL) and extracted with EA (100 mL×2). The combined organic layers were dried over Na₂SO₄, filtered and concentrated under reduced pressure to give a residue. The residue was purified by flash silica gel chromatography (PE/EA=100/1 to 1/1) to give 4-bromo-N-isopropyl-benzene-1,2-diamine (850 mg, 3.71 mmol, 48.06% yield) as a black-brown solid.

¹H NMR (400 MHz, CHLOROFORM-d) δ=6.82 (dd, J=2.1, 8.4 Hz, 1H), 6.76 (d, J=2.2 Hz, 1H), 6.44 (d, J=8.3 Hz, 1H), 3.56-3.40 (m, 1H), 1.14 (d, J=6.4 Hz, 6H).

### Synthesis of 14-4 5-bromo-1-(propan-2-yl)-1H-1,2,3-benzotriazole

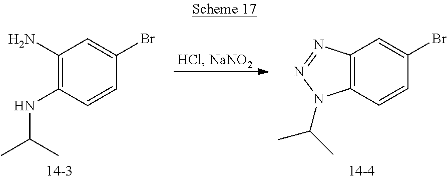

To a solution of 4-bromo-N-isopropyl-benzene-1,2-diamine (750 mg, 3.27 mmol, 1.00 eq) in HCl (5 mL, 6 M) was added NaNO₂ (271.04 mg, 3.93 mmol, 213.42 uL, 1.20 eq) in H₂O (2 mL) dropwise at 5° C. and stirred for 0.5 h. The reaction mixture was diluted with water (200 mL) and extracted with EA (200 mL×2). The combined organic layers were dried over N₄SO₄, filtered and concentrated under reduced pressure to give a residue. The residue was purified by flash silica gel chromatography (1/1) to give 5-bromo-1-isopropyl-benzotriazole (700 mg, 2.92 mmol, 89.06% yield) black-brown solid.

¹H NMR (400 MHz, DMSO-d₆) δ=8.32 (d, J=1.7 Hz, 1H), 7.94 (d, J=8.8 Hz, 1H), 7.60-7.60 (m, 1H), 7.67 (dd, J=1.8, 8.9 Hz, 1H), 5.32-5.16 (m, 1H), 1.62 (d, J=6.7 Hz, 6H).

### Synthesis of 14-5 1-(propan-2-yl)-5-(tetramethyl-1,3,2-dioxaborolan-2-yl)-1H-1,2,3-benzotriazole

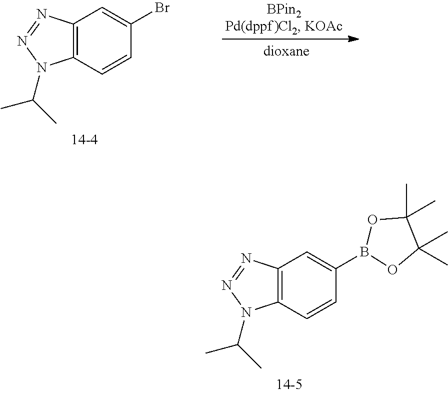 [SMILES: CC(C)n1nnc2cc(Br)ccc21 ; CC(C)n1nnc2cc(B3OC(C)(C)C(C)(C)O3)ccc21]

To a solution of 5-bromo-1-isopropyl-benzotriazole (700 mg, 2.92 mmol, 1 eq) in dioxane (10 mL) was added 4,4,5,5-tetramethyl-2-(4,4,5,5-tetramethyl-1,3,2-dioxaborolan-2-yl)-1,3,2-dioxaborolane (814.38 mg, 3.21 mmol, 1.1 eq), DPPF (161.63 mg, 291.55 umol, 0.1 eq), Pd(dppf)Cl₂ (213.33 mg, 291.55 umol, 0.1 eq) and KOAc (343.35 mg, 3.50 mmol, 1.2 eq), The mixture was stirred at 100° C. for 1 h, the mixture was diluted with water (100 mL) and extracted with (EA 100 mL×2). The combined organic layers were dried over Na₂SO₄, filtered and concentrated under reduced pressure to give a residue. The residue was purified by flash silica gel chromatography (PE/EA=100/1 to 1/1) to give 1-isopropyl-5-(4,4,5,5-tetramethyl-1,3,2-dioxaborolan-2-yl)benzotriazole (300 mg, 1.04 mmol, 35.83% yield) as a yellow solid.

¹H NMR (400 MHz, CHLOROFORM-d) δ=8.57 (s, 1H), 7.89 (dd, J=0.7, 8.3 Hz, 1H), 7.55 (dd, J=0.8, 8.4 Hz, 1H), 5.11 (spt, J=6.8 Hz, 1H), 1.75 (d, J=6.8 Hz, 6H), 1.47-1.34 (m, 12H).

### General Procedure E:

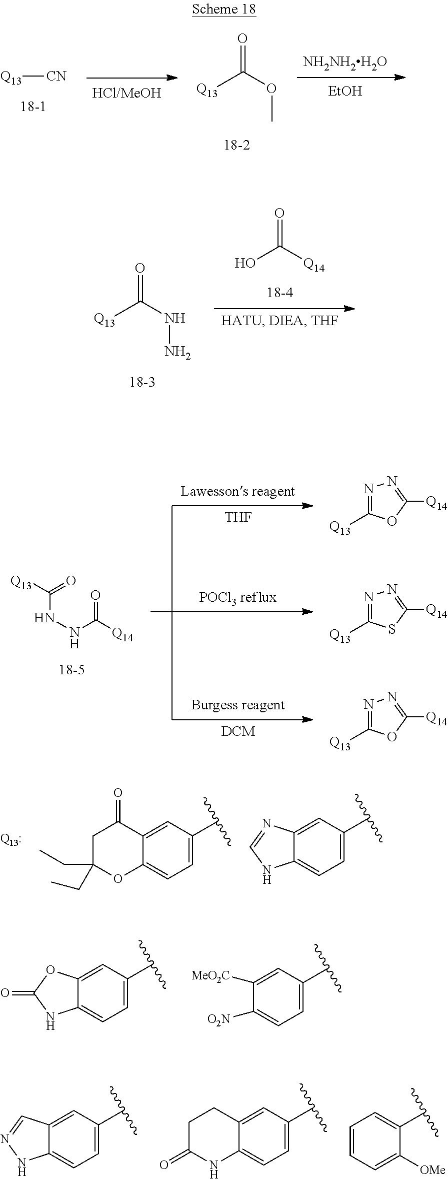

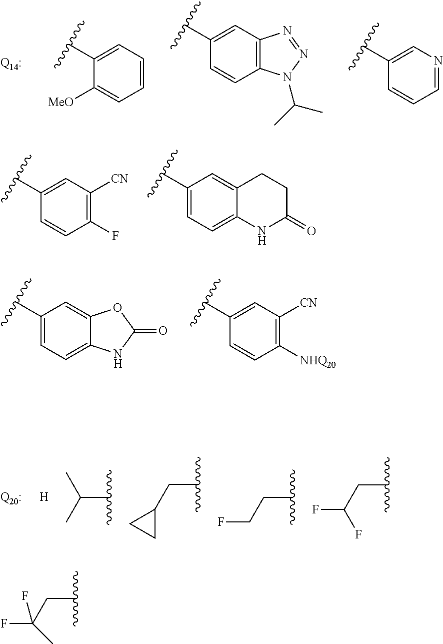

### Synthesis of 19-6 and 19-7 2,2-diethyl-6-{5-[1-(propan-2-yl)-1H-1,2,3-benzotriazol-5-yl]-1,3,4-thiadiazol-2-yl}-3,4-dihydro-2H-1-benzopyran-4-one; 2,2-diethyl-6-{5-[1-(propan-2-yl)-1H-1,2,3-benzotriazol-5-yl]-1,3,4-oxadiazol-2-yl}-3,4-dihydro-2H-1-benzopyran-4-one

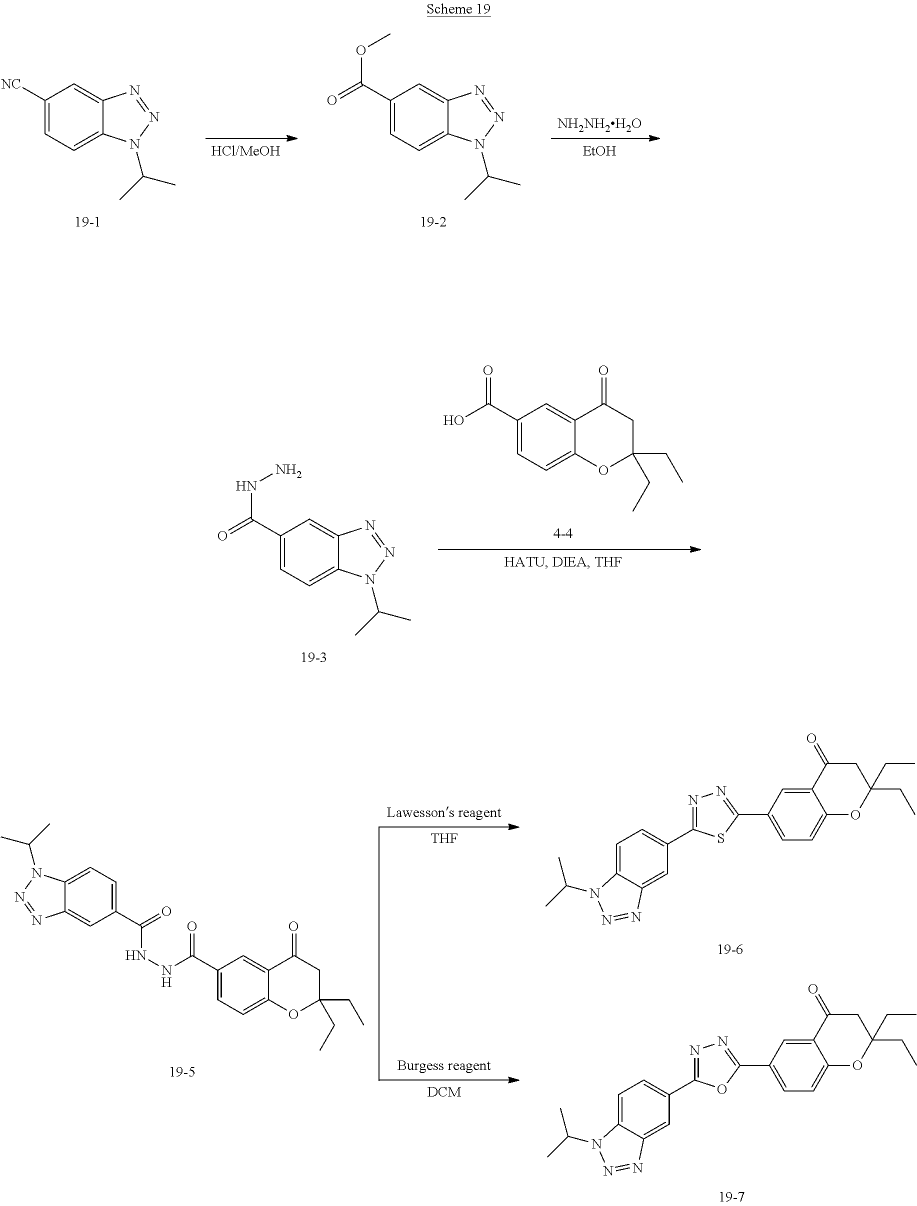

### Synthesis of 19-2: methyl 1-(propan-2-yl)-1H-1,2,3-benzotriazole-5-carboxylate

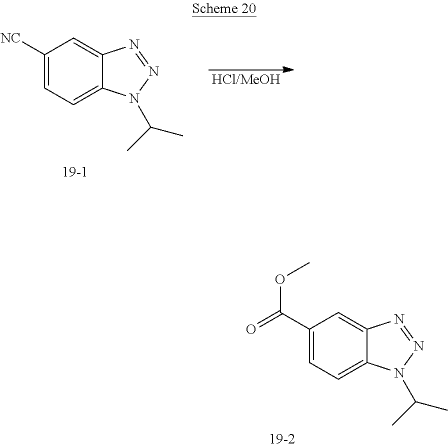 [SMILES: CC(C)n1nnc2cc(C#N)ccc21 ; COC(=O)c1ccc2c(c1)nnn2C(C)C]

A mixture of 1-isopropylbenzotriazole-5-carbonitrile (1 g, 5.37 mmol, 1 eq.) in HCl/MeOH (20 mL, 4 M) was stirred at 80° C. for 2 h. The mixture was concentrated, diluted with water (20 mL), extracted with EA (20 mL×2), dried over Na₂SO₄ and concentrated to dry. The crude product methyl 1-isopropylbenzotriazole-5-carboxylate (0.9 g, 4.11 mmol, 76.44% yield) was used into the next step without further purification.

### Synthesis of 19-3 1-(propan-2-yl)-1H-1,2,3-benzotriazole-5-carbohydrazide

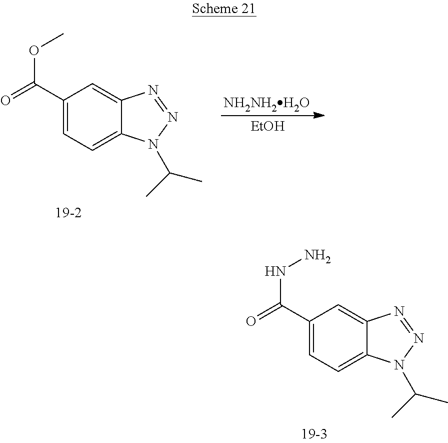

A mixture of methyl 1-isopropylbenzotriazole-5-carboxylate (0.5 g, 2.28 mmol, 1 eq.) and NH₂NH₂.H₂O (1.14 g, 22.81 mmol, 1.11 mL, 10 eq.) in EtOH (10 mL) was stirred at 80° C. for 2 h. The mixture was concentrated to dry. The residue was purified by column chromatography (SiO₂, Petroleum ether/Ethyl acetate=1:1) to give 1-isopropyl Benzotriazole-5-carbohydrazide (0.38 g, 1.73 mmol, 76.00% yield) as a white solid.

### Synthesis of 19-5 2,2-diethyl-4-oxo-N′-[1-(propan-2-yl)-1H-1,2,3-benzotriazole-5-carbonyl]-3,4-dihydro-2H-1-benzopyran-6-carbohydrazide

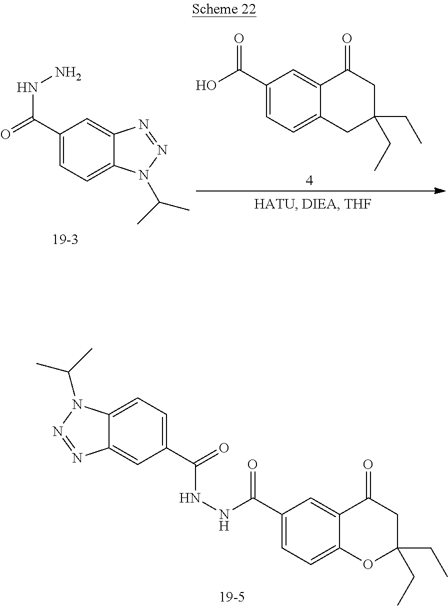 [SMILES: CC(C)n1nnc2cc(C(=O)NN)ccc21 ; CCC1(CC)CC(=O)c2cc(C(=O)O)ccc2C1 ; CCC1(CC)CC(=O)c2cc(C(=O)NNC(=O)c3ccc4c(c3)nnn4C(C)C)ccc2O1]

To a mixture of 2,2-diethyl-4-oxo-chromane-6-carboxylic acid (274.04 mg, 1.10 mmol, 1.1 eq.) and 1-isopropylbenzotriazole-5-carbohydrazide (220 mg, 1.00 mmol, 1 eq.) in THF (10 mL) was added HATU (419.70 mg, 1.10 mmol, 1.1 eq.) and DIEA (142.66 mg, 1.10 mmol, 192.26 uL, 1.1 eq.), the mixture was stirred at 15° C. for 2 hr. The mixture was diluted with water (50 mL), extracted with EA (50 mL×2), dried over Na₂SO₄ and concentrated in vacuum. N′-(2,2-diethyl-4-oxo-chromane-6-carbonyl)-1-isopropyl-benzotriazole-5-carbohydrazide (420 mg, 934.37 umol, 93.12% yield) was obtained as yellow solid without further purification.

### Synthesis of 19-7: 2,2-diethyl-6-{5-[1-(propan-2-yl)-1H-1,2,3-benzotriazol-5-yl]-1,3,4-thiadiazol-2-yl}-3,4-dihydro-2H-1-benzopyran-4-one

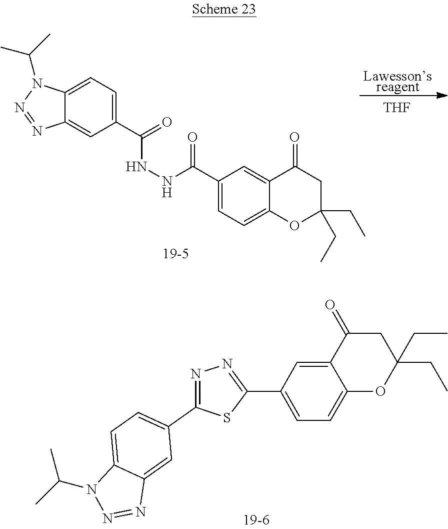

A mixture of N′-(2,2-diethyl-4-oxo-chromane-6-carbonyl)-1-isopropyl-benzotriazole-5-carbohydrazide (200 mg, 444.94 umol, 1 eq.) and Lawesson's reagent (359.93 mg, 889.88 umol, 2 eq.) in THF (2 mL) was stirred at 80° C. for 2 h. The mixture was diluted with water (20 mL), extracted with EA (20 mL×2), dried over Na₂SO₄ and concentrated to dry. The residue was purified by prep-HPLC (column: Phenomenex Synergi C18 150×25×10 um; mobile phase: [water (0.1% TFA)-ACN]; B %: 62%-92%, 13 min) to give 2,2-diethyl-6-[5-(1-isopropylbenzotriazol-5-yl)-1,3,4-thiadiazol-2-yl]chroman-4-one (61 mg, 29.99 umol, 6.74% yield, 22% purity) as a white solid.

¹H NMR (400 MHz, CHLOROFORM-d) δ=8.62 (d, J=0.6 Hz, 1H), 8.38-8.36 (m, 1H), 8.36-8.33 (m, 1H), 8.33-8.30 (m, 1H), 7.72 (d, J=8.8 Hz, 1H), 7.13 (d, J=8.7 Hz, 1H), 5.22-5.11 (m, 1H), 2.82 (s, 2H), 1.95-1.83 (m, 4H), 1.82-1.80 (m, 6H), 0.99 (t, J=7.5 Hz, 6H).

### Synthesis of 19-7: 2,2-diethyl-6-{5-[1-(propan-2-yl)-1H-1,2,3-benzotriazol-5-yl]-1,3,4-oxadiazol-2-yl}-3,4-dihydro-2H-1-benzopyran-4-one

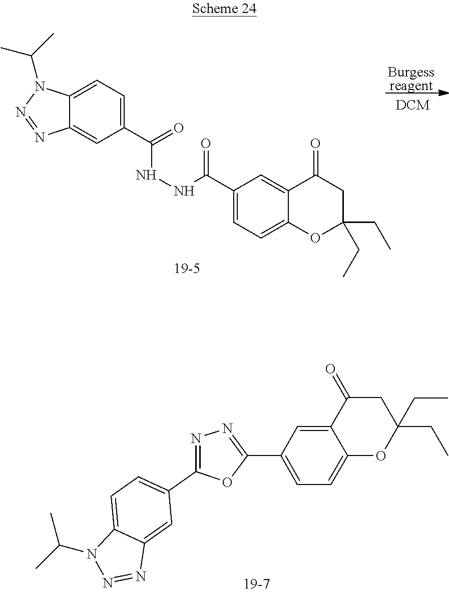

A mixture of N′-(2,2-diethyl-4-oxo-chromane-6-carbonyl)-1-isopropyl-benzotriazole-5-carbohydrazide (200 mg, 444.94 umol, 1 eq.) and Burgess reagent (530.17 mg, 2.22 mmol, 5 eq.) in DCM (2 mL) was stirred at 15° C. for 2 h. The mixture was diluted with water (20 mL), extracted with EA (20 mL×2), dried over Na₂SO₄ and concentrated to dry. The residue was purified by prep-HPLC (column: Phenomenex Synergi C18 150×25×10 um; mobile phase: [water (0.1% TFA)-ACN]; B %: 55%-85%, 12 min) to give 2,2-diethyl-6-[5-(1-isopropylbenzotriazol-5-yl)-1,3,4-oxadiazol-2-yl]chroman-4-one (56 mg, 129.78 umol, 29.17% yield, 100% purity) as a white solid.

¹H NMR (400 MHz, CHLOROFORM-d) δ=8.85 (s, 1H), 8.61 (d, J=2.3 Hz, 1H), 8.35 (t, J=2.1 Hz, 1H), 8.33 (t, J=2.1 Hz, 1H), 7.75 (d, J=8.7 Hz, 1H), 7.16 (d, J=8.8 Hz, 1H), 5.24-5.11 (m, 1H), 2.84 (s, 2H), 1.94-1.85 (m, 2H), 1.82 (d, J=6.8 Hz, 5H), 1.80-1.75 (m, 2H), 1.00 (t, J=7.5 Hz, 6H)

### Synthesis of 25-2: 5-[5-(1H-1,3-benzodiazol-5-yl)-1,3,4-oxadiazol-2-yl]-2-fluorobenzonitrile

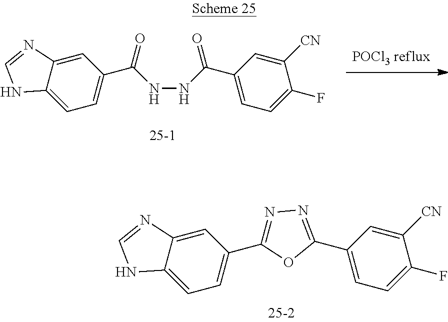 [SMILES: N#Cc1cc(C(=O)NNC(=O)c2ccc3[nH]cnc3c2)ccc1F ; N#Cc1cc(-c2nnc(-c3ccc4[nH]cnc4c3)o2)ccc1F]

A suspension of 1 (N′-(1H-1,3-benzodiazole-5-carbonyl)-3-cyano-4-fluorobenzohydrazide, 541 mg, 1.67 mmol) in phosphorus oxychloride (10 mL, excess) was heated at 105° C. for 3 hours. The mixture was concentrated, and the residue was suspended in water with sonication/stirring. The resulting solid was collected, washed with sat'd NaHCO₃and water, then dried under N2/vac in the filter funnel. The tan solid was suspended in MCCN and concentrated twice to remove residual water and dried under high vacuum to yield 25-2 (0.55 g, 107%). MH+=306.1.

### Synthesis of 26-2 6-[5-(2-methoxyphenyl)-1,3,4-oxadiazol-2-yl]-3-methyl-2,3-dihydro-1,3-benzoxazol-2-one

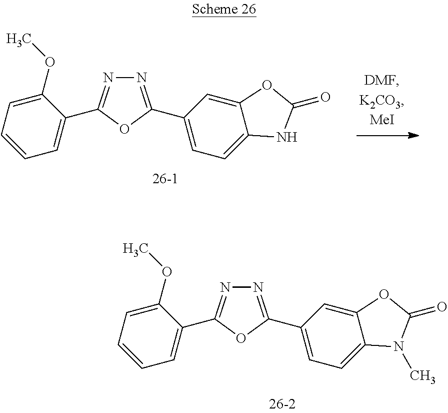 [SMILES: COc1ccccc1-c1nnc(-c2ccc3[nH]c(=O)oc3c2)o1 ; COc1ccccc1-c1nnc(-c2ccc3c(c2)oc(=O)n3C)o1]

To a suspension of 3 (6-[5-(2-methoxyphenyl)-1,3,4-oxadiazol-2-yl]-2,3-dihydro-1,3-benzoxazol-2-one trifluoroacetate, 13 mg, 0.031 mmol) in DMF (1 mL) was added potassium carbonate (8.6 mg, 0.062 mmol) followed by methyl iodide (5.2 mg, 0.037 mmol) and the resulting white suspension heated to 100° C. for 45 min. The reaction was cooled to room temperature, filtered, and purified by reverse phase chromatography, 25%-75% MeCN/water/0.1% TFA. The product fractions lyophilized to yield 26-2 (1.6 mg, 12%). MH+=324.1. ¹H NMR (400 MHz, DMSO) 7.93-7.91 (3H, m), 7.59-7.54 (1H, m), 7.44-7.41 (1H, m), 7.25-7.22 (1H, m), 7.11-7.07 (1H, m), 3.88 (3H, s); 3.45 (3H, s)

### Synthesis of 27-3 methyl N-({6-[5-(2-methoxyphenyl)-1,3,4-oxadiazol-2-yl]-2-oxo-2,3-dihydro-1,3-benzoxazol-3-yl}sulfonyl)carbamate

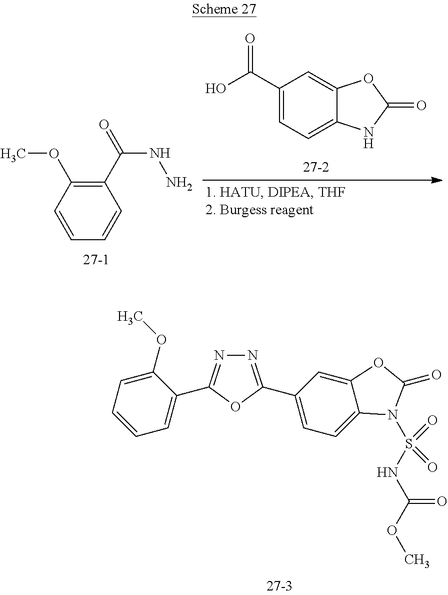 [SMILES: COc1ccccc1C(=O)NN ; O=C(O)c1ccc2[nH]c(=O)oc2c1 ; COC(=O)NS(=O)(=O)n1c(=O)oc2cc(-c3nnc(-c4ccccc4OC)o3)ccc21]

To a dry mixture of 27-1 (2-methoxyphenylhydrazide, 139 mg, 0.837 mmol), 27-2 (benzoxazol-2-one-6-carboxylic acid, 150 mg, 0.837 mmol), and HATU (318 mg, 0.837 mmol) was added THF (10 mL) to yield a hazy reddish solution. DIPEA (0.29 mL, 1.67 mmol) was added and the reaction was stirred at rt for 2 hr. Burgess reagent (499 mg, 2.09 mmol) was added one portion, and the reaction was heated to 60° C. overnight. An additional 499 mg Burgess reagent was added and continued heating. After 4 hr, 2N KHSO₄ (10 mL) was added and the resulting oily mixture was extracted 3× EtOAc. The combined organics were washed once with water, once with brine, filtered through cotton and concentrated to an orange solid which was purified by reverse phase chromatography, 20%-60% MeCN/water/0.1% TFA to yield 67 mg 27-3(18%) MH+=447.0. ¹H NMR (400 MHz, DMSO) 8.02-7.97 (3H, m), 7.74 (1H, d, J=8.4 Hz), 7.64 (1H, t, J=8.2 Hz), 7.30 (1H, d, J=8.4 Hz), 7.18 (1H, t, J=7.4 Hz), 3.95 (3H, s), 3.39 (3H, s).

### Synthesis of 28-2: 2-[(2-fluoropropyl)amino]-5-[5-(2-oxo-1,2,3,4-tetrahydroquinolin-6-yl)-1,3,4-oxadiazol-2-yl]benzonitrile

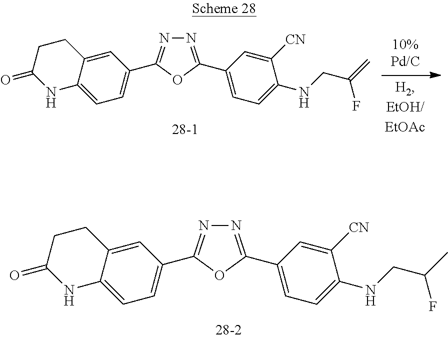 [SMILES: C=C(F)CNc1ccc(-c2nnc(-c3ccc4c(c3)CCC(=O)N4)o2)cc1C#N ; CC(F)CNc1ccc(-c2nnc(-c3ccc4c(c3)CCC(=O)N4)o2)cc1C#N]

To a nitrogen purged small Parr hydrogenation bottle was added 10% Pd/C (14 mg) and moistened with a small volume of EtOH. To 28-1 (2-[(2-fluoroprop-2-en-1-yl)amino]-5-[5-(2-oxo-1,2,3,4-tetrahydroquinolin-6-yl)-1,3,4-oxadiazol-2-yl]benzonitrile trifluoroacetate, 70 mg, 0.139 mmol) was added EtOH (10 mL) and EtOAc (90 mL) to yield a milky mixture which was added to the hydrogenation bottle. The mixture was hydrogenated under 50 psi H2 for 24 hr. MCCN was added until the milky mixture clears, then filtered through Celite and concentrated. The residue was heated in a small volume of DMF, cooled, filtered, and purified by reverse phase chromatography, 30%-75% MeCN/water/0.1% TFA. The product fractions were lyophilized to yield 28-2 as a fluffy white solid (12.8 mg, 18%). MH+=392.1. ¹H NMR (400 MHz, DMSO) 10.40 (1H, s), 8.25 (1H, d, J=2.1 Hz), 8.11 (1H, dd, J=2.0, 9.2 Hz), 7.92-7.81 (2H, m), 7.22 (1H, t, J=6.3 Hz), 7.11 (1H, d, J=9.2 Hz), 7.11 (1H, d, J=9.4 Hz), 7.02 (1H, d, J=8.7 Hz), 7.04-7.00 (1H, m), 5.00-4.80 (1H, m), 3.60-3.51 (2H, m), 3.01 (2H, t, J=7.4 Hz), 1.35 (3H, dd, J=6.2, 24.0 Hz).

### Synthesis of 29-3: 5-[5-(2-methoxyphenyl)-1,3,4-oxadiazol-2-yl]-1,3-dihydro-2,1-benzoxazol-3-one

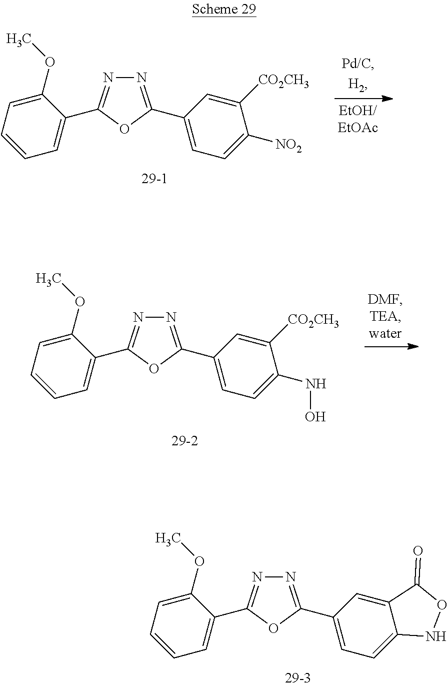 [SMILES: COC(=O)c1cc(-c2nnc(-c3ccccc3OC)o2)ccc1[N+](=O)[O-] ; COC(=O)c1cc(-c2nnc(-c3ccccc3OC)o2)ccc1NO ; COc1ccccc1-c1nnc(-c2ccc3[nH]oc(=O)c3c2)o1]

To a nitrogen purged hydrogenation bottle was added 10% Pd/C (12 mg), which was moistened with EtOH. A suspension of 29-1 (methyl 5-[5-(2-methoxyphenyl)-1,3,4-oxadiazol-2-yl]-2-nitrobenzoate, prepared according to Wuxi 1,3,4-oxadiazole experimental, 112 mg, 0.281) in EtOH (25 mL) and EtOAc (20 mL) was hydrogenated at 48 psi H2. After 30 minutes the reaction was filtered and concentrated to solid 29-2 (113 mg, 105%), which was used without further purification. MH+=342.1.

To a solution of 29-2 (47 mg, 0.132 mmol) in DMF (2 mL) was added triethylamine (0.1 mL) then water (0.2 mL) and the resulting solution stirred at rt for 60 hr. The reaction mixture was purified directly by reverse phase chromatography, 20%-65% MeCN/water/0.1% TFA to yield 8 mg 29-3 (14%). MH+=310.1. ¹H NMR (400 MHz, DMSO) 12.52 (1H, s), 8.42-8.38 (2H, m), 8.03 (1H, d, J=6.9 Hz), 7.65 (1H, t, J=8.3 Hz), 7.57 (1H, d, J=7.6 Hz), 7.32 (1H, d, J=8.3 Hz), 7.17 (1H, t, J=6.9 Hz), 3.96 (3H, s).

### Synthesis of 30-1: 5-[5-(2-methoxyphenyl)-1,3,4-oxadiazol-2-yl]-1-methyl-1,3-dihydro-2,1-benzoxazol-3-one

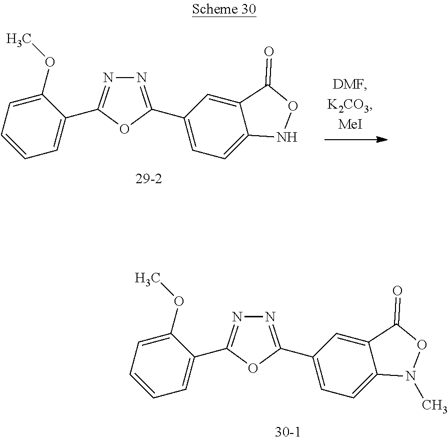 [SMILES: COc1ccccc1-c1nnc(-c2ccc3[nH]oc(=O)c3c2)o1 ; COc1ccccc1-c1nnc(-c2ccc3c(c2)c(=O)on3C)o1]

To a solution of 29-2 (8 mg, 0.019 mmol) in DMF (0.5 mL) was added potassium carbonate (2.6 mg, 0.019 mmol) followed by methyl iodide (4.0 mg, 0.028 mmol) and the resulting solution heated to 100° C. for 15 minutes. The reaction was cooled to rt, filtered, and purified directly by reverse phase chromatography, 25%-75% MeCN/water/0.1% TFA to yield 5.3 mg 30-1 (64%) as a white solid.

MH+=324.1. ¹H NMR (400 MHz, DMSO) 8.40 (1H, d, J=8.7 Hz), 8.32 (1H, s), 7.97 (1H, d, J=8.0 Hz), 7.73 (1H, d, J=8.7 Hz), 7.58 (1H, t, J=8.3 Hz), 7.25 (1H, d, J=8.0 Hz), 7.10 (1H, t, J=7.7 Hz), 3.89 (3H, s), 3.49 (3H, s).

### General Procedure F:

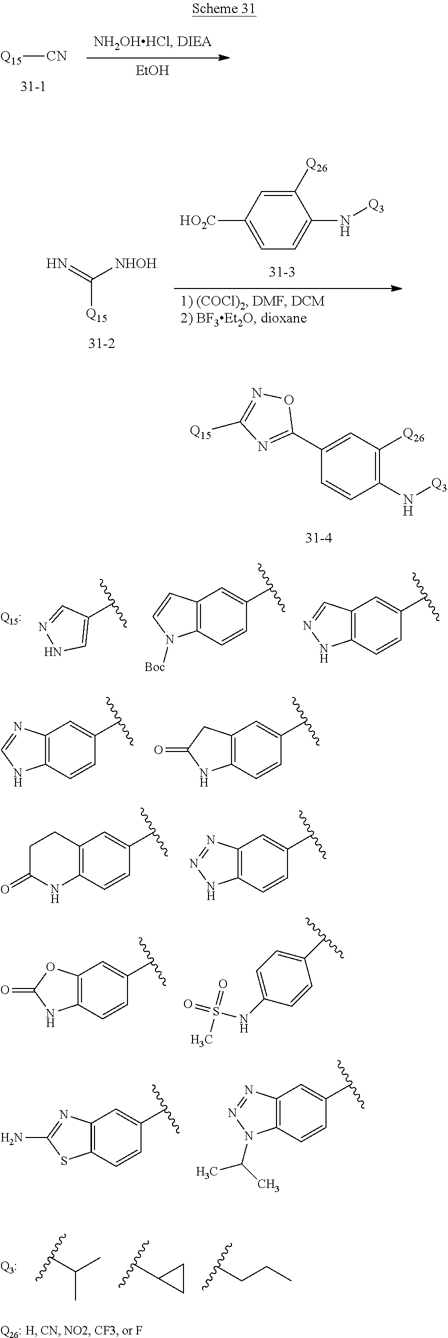

### Synthesis of 32-2 N-hydroxy-2-oxo-2,3-dihydro-1H-indole-5-carboximidamide

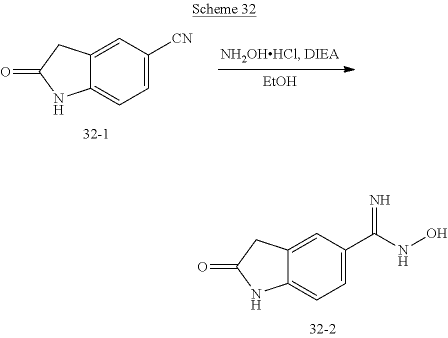 [SMILES: "EtOH" ; N#Cc1ccc2c(c1)CC(=O)N2 ; N=C(NO)c1ccc2c(c1)CC(=O)N2]

To a mixture of 2-oxoindoline-5-carbonitrile (200 mg, 1.26 mmol, 1 eq) in ethanol (5 mL) was added hydrochloride salt of hydroxylamine (176 mg, 2.53 mmol, 2.0 eq), diisopropylethylamine (327 mg, 2.53 mmol, 2.0 eq) at 20° C. under nitrogen atmosphere. The mixture was then heated to 90° C. and stirred for 16 hrs. The mixture was concentrated in vacuum, and a white solid was precipitated out. The suspension was filtered, and the white solid was dried in vacuum to give the product 32-2 (210 mg, 83% yield).

¹H NMR (400 MHz, DMSO-d₆) δ=10.46 (br, s, 1H), 9.45 (br, s, 1H), 7.51 (s, 1H), 7.50 (d, J=10.8 Hz, 1H), 6.79 (d, J=8.0 Hz, 1H), 5.70 (br, s, 2H), 3.49 (s, 2H).

### Synthesis of 33-1: 2-[(cyclopropylmethyl)amino]-5-[3-(2-oxo-2,3-dihydro-1H-indol-5-yl)-1,2,4-oxadiazol-5-yl]benzonitrile

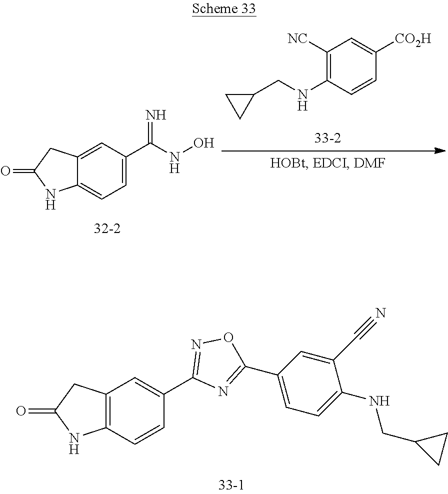 [SMILES: N=C(NO)c1ccc2c(c1)CC(=O)N2 ; N#Cc1cc(C(=O)O)ccc1NCC1CC1 ; "HOBt, EDCI, DMF" ; N#Cc1cc(-c2nc(-c3ccc4c(c3)CC(=O)N4)no2)ccc1NCC1CC1]

To a mixture of 3-cyano-4-(cyclopropylmethylamino)benzoic acid (107 mg, 496 umol, 1.2 eq) in N,N-dimethylformamide (1 mL) was added HOBt (67.0 mg, 496 umol, 1.2 eq), EDCI (95.1 mg, 496 umol, 1.2 eq) at 20° C. The mixture was stirred for 30 min, then N-hydroxy-2-oxo-indoline-5-carboxamidine (79 mg, 413 umol, 1 eq) was added, and the resultant mixture then heated to 150° C. and stirred for 1 hour. The cooled reaction mixture was directly purified by prep-HPLC (column: Boston Green ODS 150×30 5u; mobile phase: [water (0.225% FA)-ACN]; B %: 47%-77%, 10 min) to give the product 33-1 (20 mg, 12% yield).

¹H NMR (400 MHz, DMSO-d₆) δ=10.73 (br, s, 1H), 8.23 (d, J=2.4 Hz, 1H), 8.11 (dd, J=9.2, 2.0 Hz, 1H), 7.93 (d, J=8.0 Hz, 1H), 7.89 (s, 1H), 7.17 (t, J=6.4 Hz, 1H), 7.07 (d, J=9.2 Hz, 1H), 7.00 (d, J=8.0 Hz, 1H), 3.61 (s, 2H), 3.20 (t, J=6.4 Hz, 2H), 1.16-1.13 (m, 1H), 0.52-0.47 (m, 2H), 0.33-0.29 (m, 2H).

### General Procedure G:

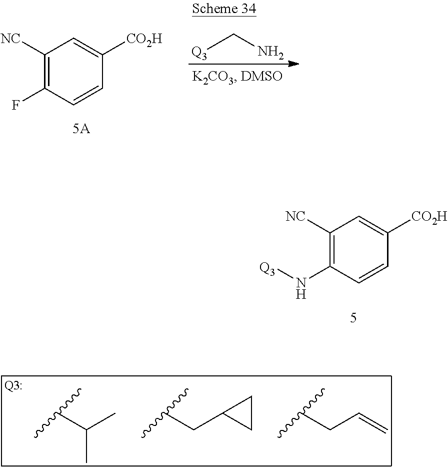

### Synthesis of 35-2 3-cyano-4-[(cyclopropylmethyl)amino]benzoic acid

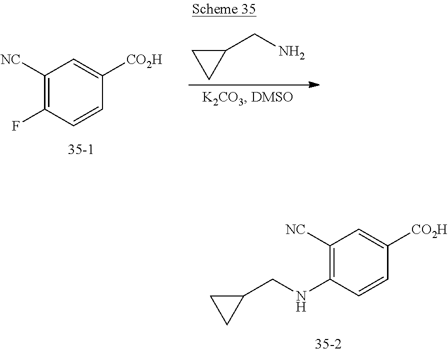

To a mixture of 3-cyano-4-fluoro-benzoic acid (5 g, 30.3 mmol, 1 eq) and cyclopropylmethanamine (5.38 g, 75.7 mmol, 2.5 eq) in dimethylsulfoxide (30 mL) was added potassium carbonate (12.6 g, 90.8 mmol, 3 eq) at 20° C. The mixture was then heated to 100° C. and stirred for 16 hours. The mixture was filtered, and the filtrate was diluted with water (50 mL), acidified by hydrochloride solution (2N) to pH=4-5. A yellow solid was precipitated out, the suspension was filtered, and the solid was washed by water (50 mL×3). The solid was dried in vacuum to give the desired product 35-2 (5 g, 73% yield). 1H NMR (400 MHz, DMSO-d₆) δ=7.95 (d, J=1.2 Hz, 1H), 7.90 (d, J=9.2 Hz, 1H), 6.88 (d, J=9.2 Hz, 1H), 6.85-6.82 (m, 1H), 3.13 (t, J=6.0, 2H), 1.16-1.06 (m, 1H), 0.49-0.44 (m, 2H), 0.29-0.25 (m, 2H).

### Synthesis of 36-2 5-[3-(1H-indol-5-yl)-1,2,4-oxadiazol-5-yl]-2-[(prop-2-en-1-yl)amino]benzonitrile

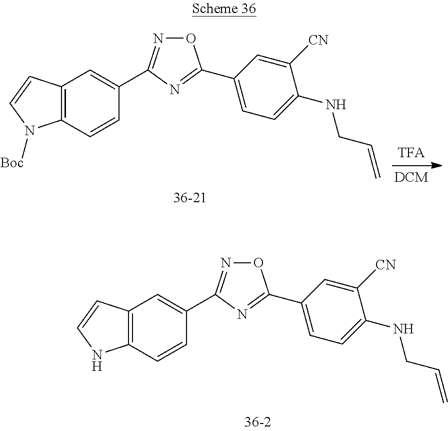 [SMILES: C=CCNc1ccc(-c2nc(-c3ccc4c(ccn4C(=O)OC(C)(C)C)c3)no2)cc1C#N ; C=CCNc1ccc(-c2nc(-c3ccc4[nH]ccc4c3)no2)cc1C#N]

To a mixture of tert-butyl 5-[5-[4-(allylamino)-3-cyano-phenyl]-1,2,4-oxadiazol-3-yl]indole-1-carboxylate (60.0 mg, 136 umol, 1.00 eq) in dichloromethane (10.0 mL) was added trifluoroacetic acid (770 mg, 6.75 mmol, 50 eq) at 20° C. under nitrogen atmosphere. The mixture was stirred at 20° C. for 16 h. The mixture was concentrated in vacuum and the residue was purified by prep-HPLC (column: Gemini 150×25 5u; mobile phase: [water (0.05% ammonia hydroxide v/v)-ACN]; B %: 47%-77%, 12 min) to give the product 36-2 (15 mg, 30% yield) as a white solid.

¹H NMR (400 MHz, DMSO-d) δ=8.33 (s, 1H), 8.27 (d, J=2.0 Hz, 1H), 8.14 (dd, J=8.8, 2.0 Hz, 1H), 7.80 (dd, J=8.4, 1.2 Hz, 1H), 7.56 (d, J=8.8 Hz, 1H), 7.48 (t, J=2.4 Hz, 1H), 7.39 (t, J=5.6 Hz, 1H), 6.91 (d, J=8.8 Hz, 1H), 6.61 (s, 1H), 5.94-5.85 (m, 1H), 5.24-5.16 (m, 2H), 3.97 (s, 1H).

### General Procedure H:

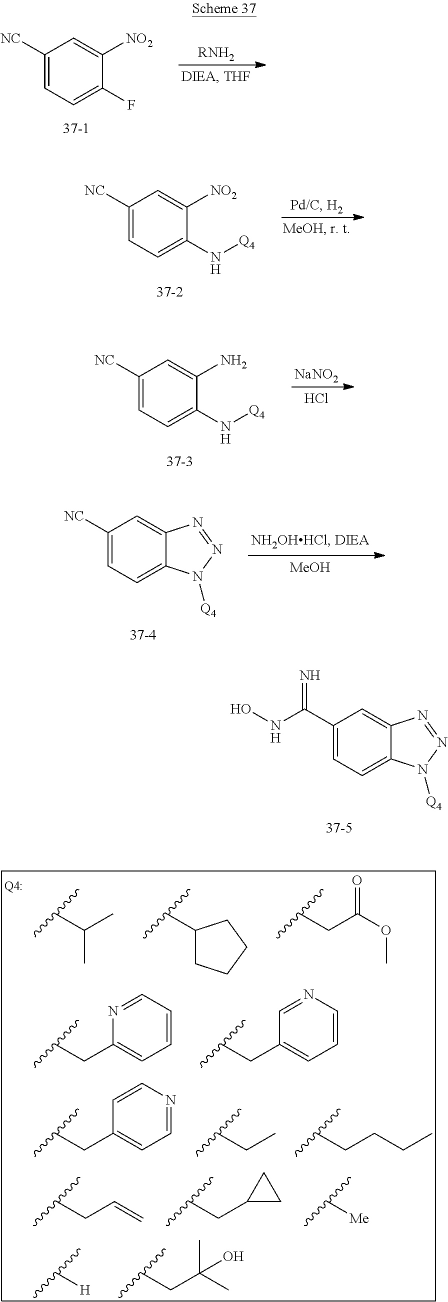

### Synthesis of 38-4 1-cyclopentyl-N-hydroxy-1H-1,2,3-benzotriazole-5-carboximidamide

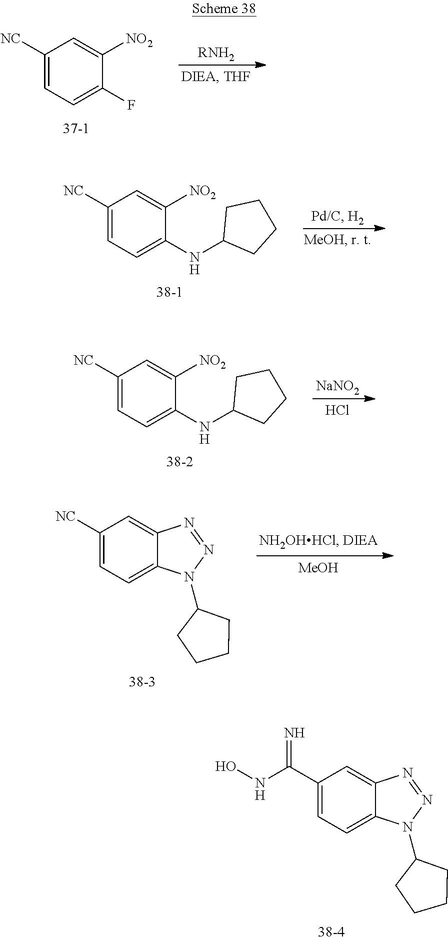

### Synthesis of 38-1 4-(cyclopentylamino)-3-nitrobenzonitrile

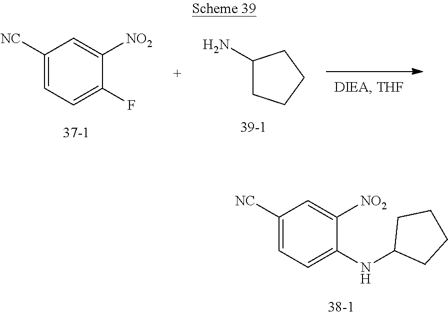 [SMILES: N#Cc1ccc(F)c([N+](=O)[O-])c1 ; NC1CCCC1 ; N#Cc1ccc(NC2CCCC2)c([N+](=O)[O-])c1]

Preparation of 1: To a solution of 4-fluoro-3-nitro-benzonitrile (1.00 g, 6.02 mmol, 1.00 eq) and cyclopentanamine (767 mg, 9.03 mmol, 1.50 eq) in THF (20.00 mL) was added DIEA (1.95 g, 15.05 mmol, 2.63 mL, 2.50 eq) and the mixture was stirred at 10° C. for 16 hour. The mixture was evaporated to dry and diluted with H₂O (50 mL), extracted with DCM (50 mL×2), dried over Na₂SO₄, filtered and concentrated to dry. Compound 4-(cyclopentylamino)-3-nitrobenzonitrile (1.36 g, 5.91 mmol, 98.10% yield) was obtained as a yellow solid which was used directly in next step.

### Synthesis of 38-2 3-amino-4-(cyclopentylamino)benzonitrile

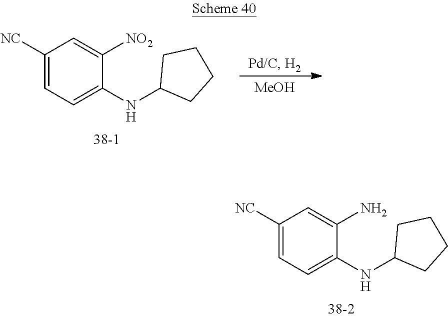 [SMILES: "MeOH" ; N#Cc1ccc(NC2CCCC2)c([N+](=O)[O-])c1 ; "Pd/C, H2" ; N#Cc1ccc(NC2CCCC2)c(N)c1]

Preparation of 2: To a solution of 4-(cyclopentylamino)-3-nitro-benzonitrile (5.00 g, 21.62 mmol, 1.00 eq) in MeOH (150.00 mL) was added Pd/C (1.00 g, 4.32 mmol, 10% purity, 0.20 eq). Then the mixture was stirred for 12 hours at 20° C. under H2 (50 psi). The mixture was filtered and the filtrate was concentrated to dry to get 3-amino-4-(cyclopentylamino)benzonitrile (4.20 g, 20.87 mmol, 96.52% yield) as black solid, which was used directly in the next step. ¹H NMR (400 MHz, DMSO-d6) δ=8.77 (s, 1H), 8.12 (d, J=8.7 Hz, 1H), 7.91 (d, J=8.7 Hz, 1H), 5.43 (m, 1H), 2.37-2.21 (m, 2H), 2.18-2.06 (m, 2H), 1.99-1.85 (m, 2H), 1.81-1.67 (m, 2H).

### Synthesis of 38-4: 1-cyclopentyl-N-hydroxy-1H-1,2,3-benzotriazole-5-carboximidamide

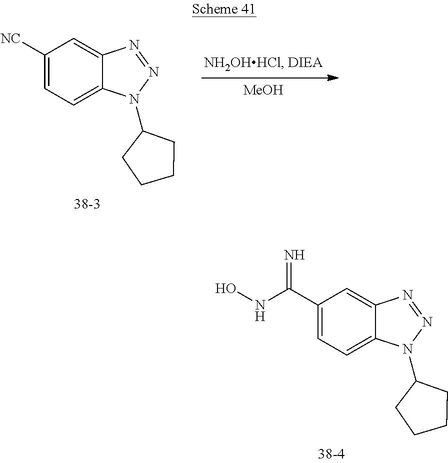 [SMILES: "MeOH" ; N#Cc1ccc2c(c1)nnn2C1CCCC1 ; N=C(NO)c1ccc2c(c1)nnn2C1CCCC1]

Preparation of 3: To a solution of 1-cyclopentylbenzotriazole-5-carbonitrile (1.50 g, 7.07 mmol) in ethanol (15.00 mL) was added hydroxylamine hydrochloride (736.64 mg, 10.60 mmol) and DIPEA (2.01 g, 15.55 mmol, 2.72 mL). Then the mixture was stirred at 70° C. for 4 hours. The mixture was filtered to get 1-cyclopentyl-N-hydroxy-benzotriazole-5-carboxamidine (1.20 g, 4.89 mmol, 69.20% yield) as a white solid.

¹H NMR (400 MHz, DMSO-d6) δ=9.77 (s, 1H), 8.31 (s, 1H), 7.97-7.89 (m, 1H), 7.87-7.79 (m, 1H), 5.98 (s, 2H), 5.34 (q, J=7.0 Hz, 1H), 2.36-2.21 (m, 2H), 2.19-2.07 (m, 2H), 1.99-1.84 (m, 2H), 1.81-1.67 (m, 2H).

### General Procedure I:

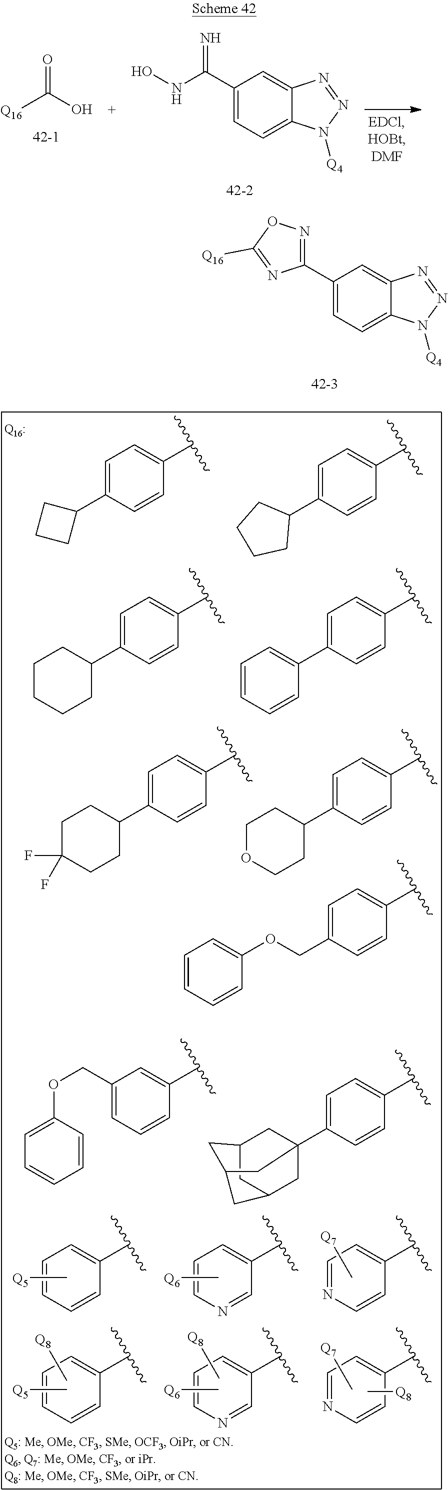

### Synthesis of 42-2 5-[5-(2-bromophenyl)-1,2,4-oxadiazol-3-yl]-1-cyclopentyl-1H-1,2,3-benzotriazole

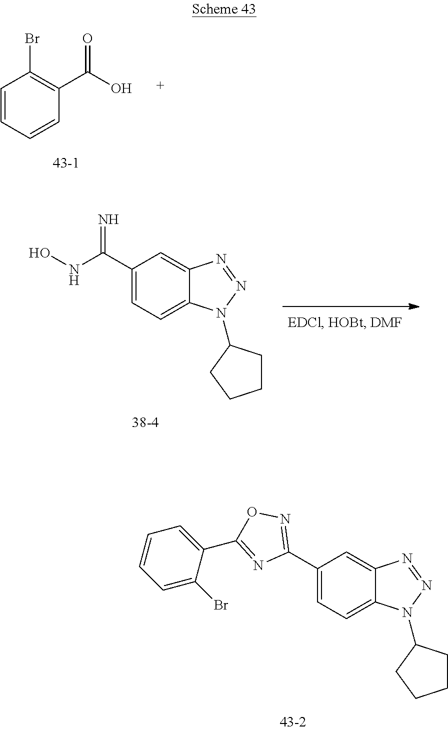 [SMILES: O=C(O)c1ccccc1Br ; N=C(NO)c1ccc2c(c1)nnn2C1CCCC1 ; Brc1ccccc1-c1nc(-c2ccc3c(c2)nnn3C2CCCC2)no1]

To a solution of 3-methylbenzoic acid (66.61 mg, 489.24 umol) in DMF (4.00 mL) was added EDCI (93.79 mg, 489.24 umol) and HOBt (66.11 mg, 489.24 umol). The mixture was stirred at 20° C. for 1 hour and 1-cyclopentyl-N-hydroxy-benzotriazole-5-carboxamidine (100.00 mg, 407.70 umol) was added to the mixture. Then the mixture was stirred at 120° C. for 12 hours under N2. The mixture was quenched with water (30 mL) and extracted with ethyl acetate (30 mL×2). The organic layers were washed with brine (40 mL), dried over Na₂SO₄ and concentrated to dryness. The residue was purified by prep-HPLC (TFA) to get t 3-(1-cyclopentylbenzotriazol-5-yl)-5-(m-tolyl)-1,2,4-oxadiazole (96.00 mg, 277.94 umol, 68.17% yield) as a white solid. ¹H NMR (400 MHz, CDCl₃) δ=8.93 (s, 1H), 8.30 (dd, J=1.3, 8.7 Hz, 1H), 8.08 (s, 1H), 8.06 (br d, J=6.8 Hz, 1H), 7.68 (d, J=8.8 Hz, 1H), 7.51-7.42 (m, 2H), 5.22 (quin, J=7.0 Hz, 1H), 2.50 (s, 3H), 2.43-2.32 (m, 4H), 2.15-2.01 (m, 2H), 1.92-1.81 (m, 2H).

### Synthesis of 44-2 2-{5-[5-(2-bromophenyl)-1,2,4-oxadiazol-3-yl]-1H-1,2,3-benzotriazol-1-yl}acetic acid

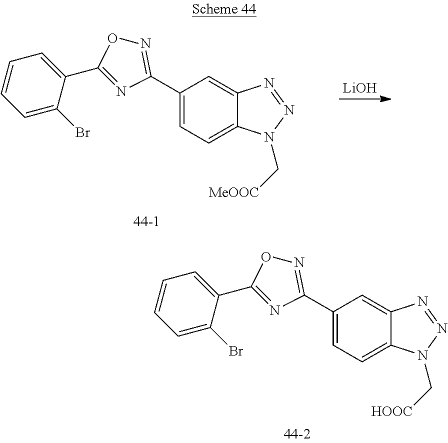 [SMILES: COC(=O)Cn1nnc2cc(-c3noc(-c4ccccc4Br)n3)ccc21 ; "LiOH" ; O=C(O)Cn1nnc2cc(-c3noc(-c4ccccc4Br)n3)ccc21]

To a solution of methyl 2-[5-[5-(2-bromophenyl)-1,2,4-oxadiazol-3-yl]benzotriazol-1-yl] acetate (40.00 mg, 96.57 umol, 1.00 eq) in dioxane (2.00 mL) and H₂O (2.00 mL) was added NaOH (15.45 mg, 386.28 umol, 4.00 eq). Then the mixture was stirred at 20° C. for 12 hours. The mixture was adjust to pH=2˜3 with HCl (1N) and extracted with ethyl acetate (15 mL×2). The organic layers were dried over Na₂SO₄ and concentrated to give 2-[5-[5-(2-bromophenyl)-1,2, 4-oxadiazol-3-yl]benzotriazol-1-yl]acetic acid (17.30 mg, 43.23 umol, 44.76% yield) as a white solid.

¹H NMR (400 MHz, DMSO-d₆) δ=8.70 (s, 1H), 8.26 (m, 3H), 7.93 (m, 2H), 7.66 (m, 2H), 5.24 (br s, 2H)

### Synthesis of 45-1 2-{5-[5-(2-bromophenyl)-1,2,4-oxadiazol-3-yl]-1H-1,2,3-benzotriazol-1-yl}ethan-1-ol

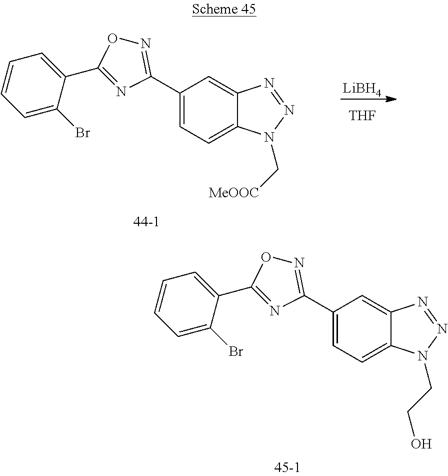 [SMILES: COC(=O)Cn1nnc2cc(-c3noc(-c4ccccc4Br)n3)ccc21 ; "LiBH4" ; OCCn1nnc2cc(-c3noc(-c4ccccc4Br)n3)ccc21 ; "THF"]

To a solution of methyl 2-[5-[5-(2-bromophenyl)-1,2,4-oxadiazol-3-yl]benzotriazol-1-yl] acetate (50.00 mg, 120.71 umol, 1.00 eq) in THF (1.00 mL) was added LiBH₄ (5.26 mg, 241.42 umol, 2.00 eq) and stirred at 10° C. for 16 h. The mixture was diluted with H₂O (20 mL) and extracted with EA (30 mL×2), the combined organic layers were dried over Na₂SO₄, filtered and concentrated to dry. The residue was purified by prep-HPLC (TFA condition) to give 2-[5-[5-(2-bromophenyl)-1,2,4-oxadiazol-3-yl]benzotriazol-1-yl]ethanol (10.00 mg, 25.89 umol, 21.45% yield) as a white solid.

¹H NMR (400 MHz, DMSO-d₆) δ=8.74 (s, 1H), 8.43 (br s, 1H), 8.25 (dd, J=1.1, 8.8 Hz, 1H), 8.17 (dd, J=1.9, 7.4 Hz, 1H), 8.12 (d, J=8.7 Hz, 1H), 8.01-7.92 (m, 1H), 7.74-7.60 (m, 2H), 4.84 (t, J=5.1 Hz, 2H), 3.93 (t, J=5.1 Hz, 2H).

### General Procedure J:

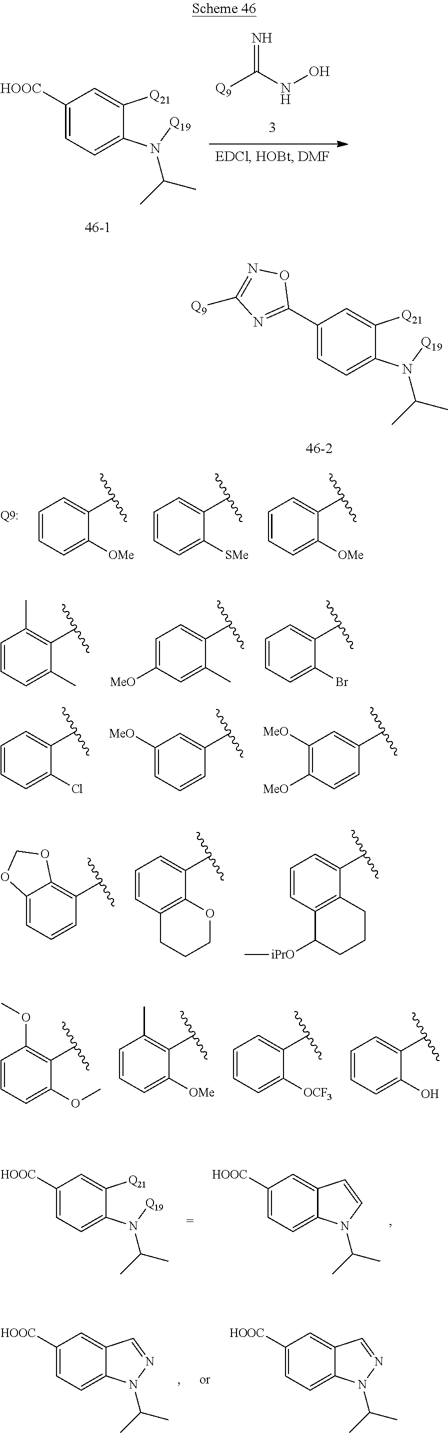

### Synthesis of 46-1 3-(2-isopropoxyphenyl)-5-(1-isopropylindol-5-yl)-1,2,4-oxadiazole

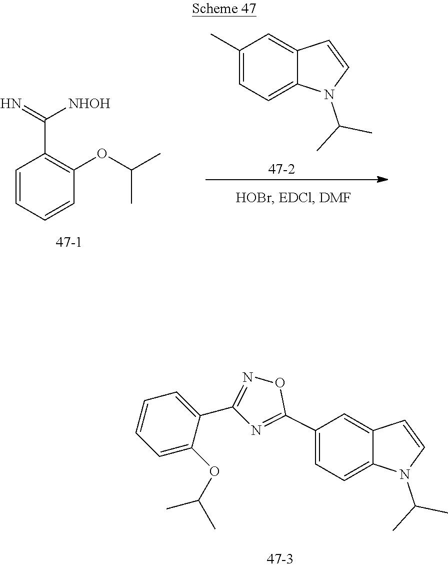

To a solution of N-hydroxy-2-isopropoxy-benzamidine (90.00 mg, 393.86 umol, 1.00 eq) in DMF (2.00 mL) was added 1-isopropylindole-5-carboxylic acid (80.05 mg, 393.86 umol, 1.00 eq), HOBt (63.86 mg, 472.63 umol, 1.20 eg) and EDCI (90.60 mg, 472.63 umol, 1.20 eq), the reaction was stirred at 120° C. for 12h. The mixture was filtered and concentrated. The residue was purified by prep-HPLC to give 3-(2-isopropoxyphenyl)-5-(1-isopropylindol-5-yl)-1,2,4-oxadiazole (26.00 mg, 71.93 umol, 18.26% yield, 98.9% purity) as a yellow oil.

¹H NMR (400 MHz, CHLOROFORM-d) δ=8.56 (d, J=1.1 Hz, 1H), 8.10 (dt, J=1.7, 8.6 Hz, 2H), 7.53-7.43 (m, 2H), 7.35 (d, J=3.3 Hz, 1H), 7.14-7.07 (m, 2H), 6.69 (d, J=3.3 Hz, 1H), 4.81-4.65 (m, 2H), 1.60 (d, J=6.7 Hz, 7H), 1.46 (d, J=6.1 Hz, 6H).

### Synthesis of 48-2 5-[3-(2-methoxyphenyl)-1,2,4-oxadiazol-5-yl]-1-(propan-2-yl)-1H-1,2,3-benzotriazole

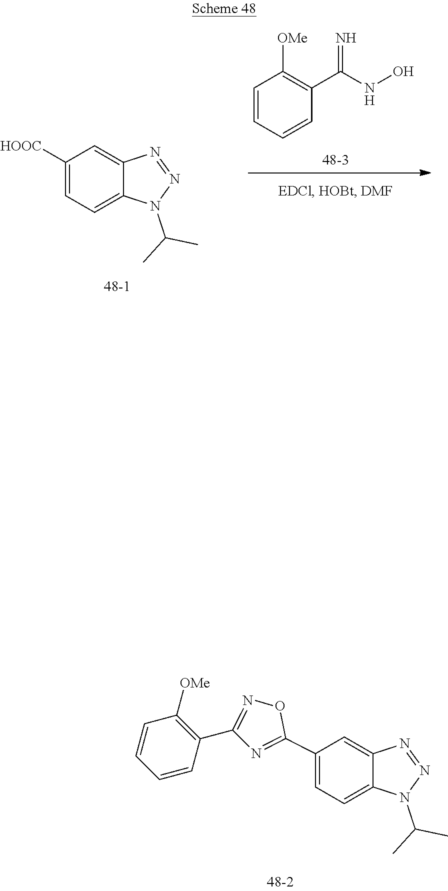 [SMILES: COc1ccccc1C(=N)NO ; CC(C)n1nnc2cc(C(=O)O)ccc21 ; COc1ccccc1-c1noc(-c2ccc3c(c2)nnn3C(C)C)n1]

To a solution of 1-isopropylbenzotriazole-5-carboxylic acid (100.00 mg, 487.31 umol, 1.00 PGP-1611 eq) in DMF (1.00 mL) was added HOBt (79.01 mg, 584.77 umol, 1.20 eq) and EDCI (112.10 mg, 584.77 umol, 1.20 eq). The mixture was stirred at 10° C. for 0.5 h, then N-hydroxy-2-methoxy-benzamidine (80.98 mg, 487.31 umol, 1.00 eq) was added and stirred at 120° C. for 12 h. The mixture was diluted with H₂O (20 mL) and extracted with EA (30 mL×2), the combined organic layers were dried over Na₂SO₄, filtered and concentrated to dry. The residue was purified by prep-HPLC (column: Welch Ultimate AQ-C18 150×30 mm×5 um; mobile phase: [water (0.1% TFA)-ACN]; B %: 50%-80%, 13 min) to give 5-(1-isopropylbenzotriazol-5-yl)-3-(2-methoxyphenyl)-1,2,4-oxadiazole (110.00 mg, 328.01 umol, 67.31% yield) as a white solid.

¹H NMR (400 MHz, CHLOROFORM-d) δ=8.99 (s, 1H), 8.36 (dd, J=1.2, 8.7 Hz, 1H), 8.17 (dd, J=1.6, 7.7 Hz, 1H), 7.74 (d, J=8.7 Hz, 1H), 7.59-7.48 (m, 1H), 7.20-7.05 (m, 2H), 5.16 (spt, J=6.8 Hz, 1H), 4.03 (s, 3H), 1.81 (d, J=6.8 Hz, 6H).

### General Procedure K:

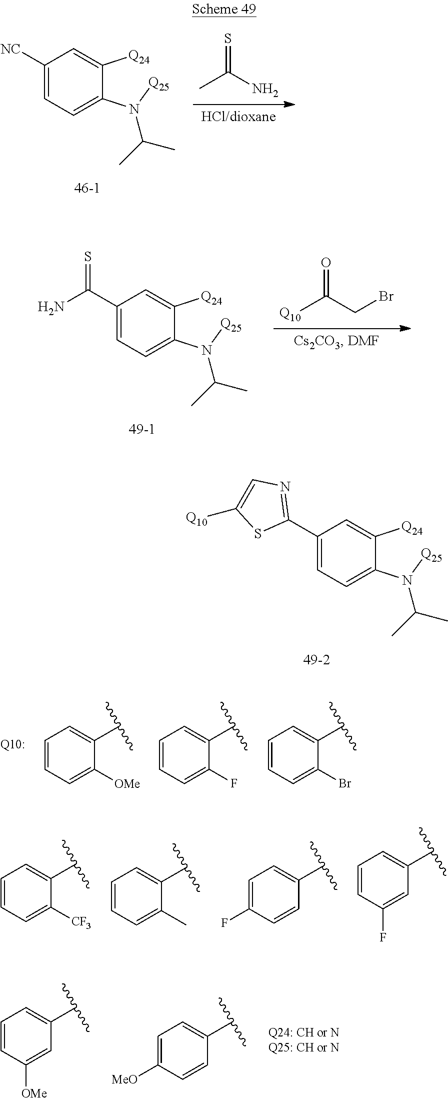

### Synthesis of 49-1: 1-(propan-2-yl)-1H-1,2,3-benzotriazole-5-carbothioamide

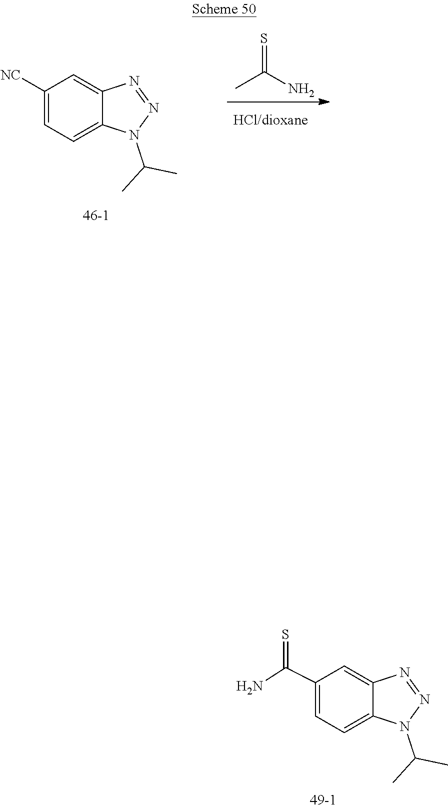

To a solution of 1-isopropylbenzotriazole-5-carbonitrile (2 g, 10.74 mmol, 1 eq) in HCl/dioxane (100 mL, 4 M) was added thioacetamide (1.61 g, 21.48 mmol, 2 eq) and stirred at 110° C. for 2 h. The reaction mixture was concentrated under reduced pressure and purified by flash silica gel chromatography (PE/EA=2/1 to 1/1) to give 1-isopropylbenzotriazole-5-carbothioamide (2.1 g, 9.53 mmol, 88.76% yield) as a yellow solid.

### Synthesis of 51-25-{5-[2-(methyl-1′-oxy)phenyl]-1,3-thiazol-2-yl}-1-(propan-2-yl)-1H-1,2,3-benzotriazole

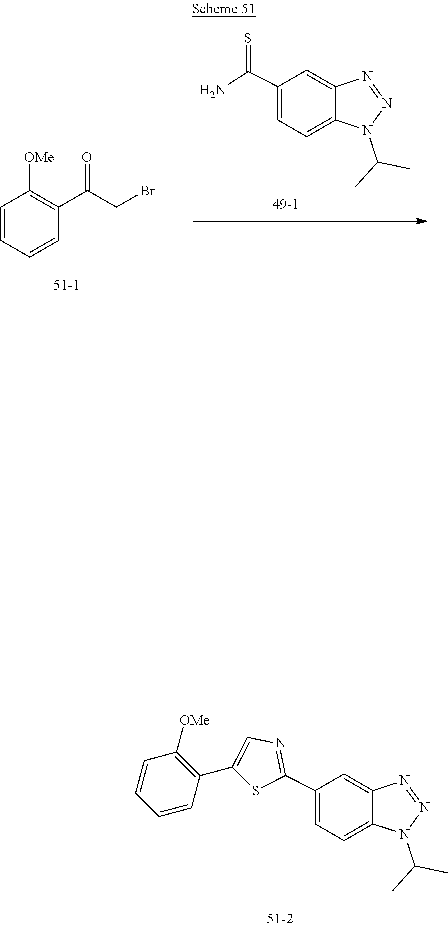 [SMILES: COc1ccccc1C(=O)CBr ; CC(C)n1nnc2cc(C(N)=S)ccc21 ; COc1ccccc1-c1cnc(-c2ccc3c(c2)nnn3C(C)C)s1]

To a solution of 1-isopropylbenzotriazole-5-carbothioamide (300 mg, 1.36 mmol, 1 eq) in DMF (3 mL) was added Cs₂CO₃ (443.71 mg, 1.36 mmol, 1.00 eq) and 2-bromo-1-(2-methoxyphenyl)ethanone (311.95 mg, 1.36 mmol, 1 eq). The mixture was stirred at 100° C. for 12 h. The residue was purified by prep-HPLC (column: Waters Xbridge 150×25 5 u; mobile phase: [water (10 mM NH₄HCO₃)-ACN]; B %: 60%-90%, 10 min) to give 2-(1-isopropylbenzotriazol-5-yl)-5-(2-methoxyphenyl)thiazole (190 mg, 509.65 umol, 37.42% yield, 94% purity) as a yellow solid.

¹H NMR (400 MHz, CHLOROFORM-d) δ=8.60 (s, 1H), 8.37 (dd, J=1.7, 7.8 Hz, 1H), 8.22 (dd, J=1.1, 8.8 Hz, 1H), 7.91 (s, 1H), 7.55 (d, J=8.8 Hz, 1H), 7.31-7.23 (m, 1H), 7.05 (t, J=7.5 Hz, 1H), 6.96 (d, J=8.3 Hz, 1H), 5.04 (spt, J=6.8 Hz, 1H), 3.92 (s, 3H), 1.70 (d, J=6.7 Hz, 6H).

### General Procedure L:

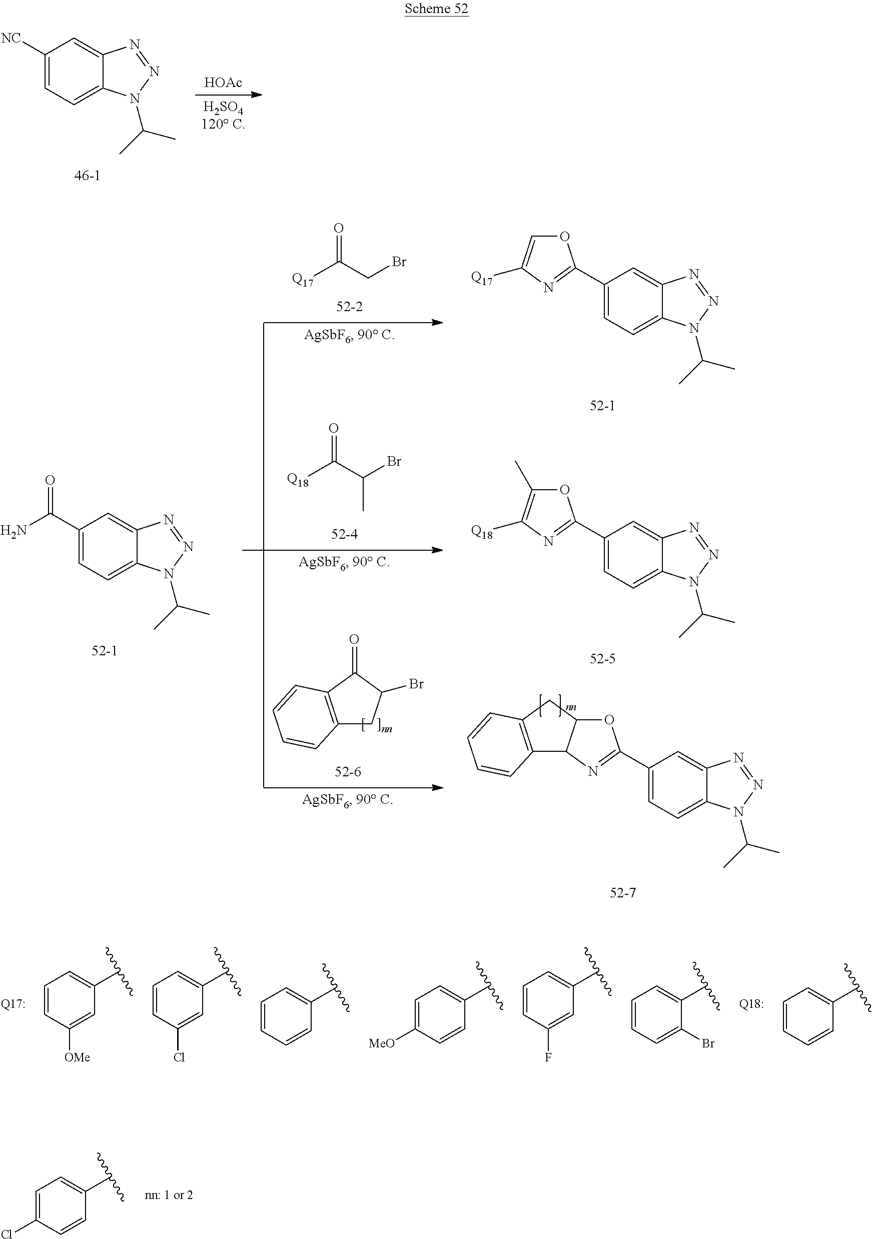

### Synthesis of 52-1: 1-(propan-2-yl)-1H-1,2,3-benzotriazole-5-carboxamide

To a solution of 46-1 (1.0 g, 5.4 mmol) in HOAc (10 mL) was added H2SO4 (0.5 mL) and reaction heated in MW 90 min at 120° C. Let cool overnight. Reaction mixture poured onto ice, neutralized and extracted with EtOAc. Solvent evaporated to give a dark solid. Silica gel chromatography (10-50% acetone in hexane) gave a residue which was triturated with a small amount of acetone and an off-white solid was collected by filtration to give the title compound (52-1) (0.5 g, 45%).

### Synthesis of 52-3: 5-(4-phenyl-1,3-oxazol-2-yl)-1-(propan-2-yl)-1H-1,2,3-benzotriazole

To a mixture of 52-1 (50 mg, 0.25 mmol), 52-2 (51 mg, 0.25 mmol) was added AgSbF6 (86 0.25 mmol) and the mixture was heated to 90° C. for about 3 hrs then cool to rt. Reaction worked up with NaHCO3and CH2Cl2. Organic layer separated and evaporated to give a dark oil. Residue was chromatographed (0 to 5% MeOH in CH2Cl2) to give a residue which was further purified by reverse phase HPLC. Appropriate fractions combined and lyophilized to give the title compound as an off-white solid. (7 mg, 10%).

### General Procedure M

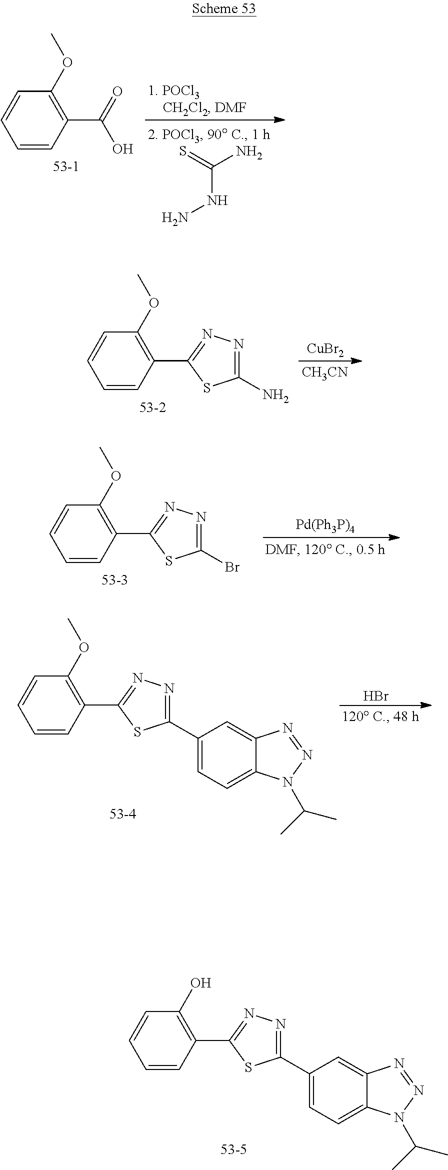

### Synthesis of 5-(2-methoxyphenyl)-1,3,4-thiadiazol-2-amine 53-2

To a solution of 53-1 (1.9 g, 10.0 mmol) in CH2Cl2 (50 mL) was added DMF (0.2 mL) followed by portion wise addition of oxalyl chloride (1.7 mL, 20.0 mmol) and the solution was stirred overnight at rt. Reaction mixture was evaporated in vacuum. To residue was added thiosemicarbazide (1.1 g, 15 mmol) followed by POCl3 (2.8 mL, 30 mmol) and the reaction mixture was heated to 90° C. After about 45 min to 1 hr, heat was turned off and allowed to cool overnight. Quench with ice and worked up with K2CO3 and EtOAc. Organic layer washed with sat'd NaHCO₃and dried over Na2SO4. Filtered and evaporated to give a yellow residue which was triturated with CH2Cl2 and the title compound was collected as a beige solid (0.9 g, 43%). This material was used directly in the next step.

### Synthesis of 2-bromo-5-(2-methoxyphenyl)-1,3,4-thiadiazole 53-3

A mixture of t-Bu-nitrite (0.9 mL, 9.6 mmol) and CuBr2 (2.2 g, 9.6 mmol) in MCCN (30 mL) was stirred for 10 min and then 53-2 (0.9 g, 1.76 mmol) was added in 2 portions. Stirred ˜1 hr and then solvent removed in vacuum. Residue was suspended in EtOAc, washed 2× with 1 N HCl, then brine and dried over Na2SO4. Filtered and evaporated to give the title compound as a yellow-orange solid (1 g, 86%).

### Synthesis of 5-[5-(2-methoxyphenyl)-1,3,4-thiadiazol-2-yl]-1-(propan-2-yl)-1H-1,2,3-benzotriazole 53-4

Combined 52-3 (95 mg, 0.25 mmol), 14-5 (45 mg, 0.3 mmol), K3PO4 (132 mg, 0.625 mmol) and Pd(Ph3P)4 (58 mg, 0.05 mmol) in a mixture of DMF (4 mL) and water (1 mL). Heated to 120° C. for 30 min in MW. Evap sol to give a residue which was chromatographed on silica (0 to 30% EtOAc in hex). Appropriate fraction were combined and solvent evaporated. This residue was further purified by reverse phase HPLC to give the title compound (30 mg, 20%) as an off-white solid.

### Synthesis of 2-{3-[1-(propan-2-yl)-1H-1,2,3-benzotriazol-5-yl]-1,2,4-thiadiazol-5-yl}phenol 53-5

53-4 (40 mg, 0.11 mmol) was dissolved in HR and heated to 120° C. After ˜48 hrs starting material was gone. Neutralize with NaHCO₃. Residue purified by reverse phase HPLC to give the title compound (10 mg, 22%). [M+H]⁺: 338.0

### General Procedure N

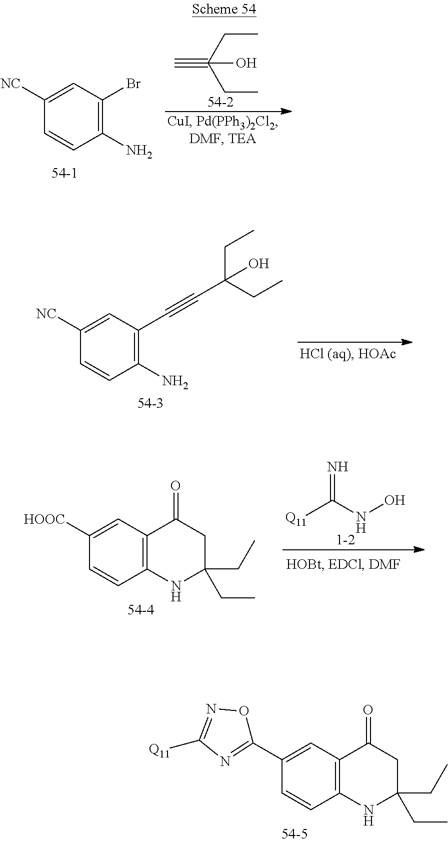

### Synthesis of 54-3: 4-amino-3-(3-ethyl-3-hydroxypent-1-yn-1-yl)benzonitrile

To a solution of compound 54-1 (1 g, 5.08 mmol, 1 eq.) in DMF (7 mL) and TEA (2.18 g, 21.55 mmol, 3 mL, 4.25 eq.) was added compound 54-2 (683.15 mg, 6.09 mmol, 783.43 uL, 1.2 eq.), CuI (48.33 mg, 253.77 umol, 0.05 eq.) and Pd(PPh₃)₂Cl₂ (178.12 mg, 253.77 umol, 0.05 eq.). The mixture was stirred at 90° C. under nitrogen atmosphere for 3 hours. TLC showed a new one spot formed. The reaction mixture was diluted with water (20 mL), extracted with EtOAc (20 mL×2), washed with brine (20 mL), dried with sodium sulfate, filtered and concentrated. The residue was purified by flash silica gel column chromatography (PE:EA=5:1) to give compound 3 (1 g, yield: 86%) as a yellow oil. ¹H NMR (400 MHz, CDCl₃) δ=7.52 (d, J=1.6 Hz, 1H), 7.34 (dd, J=8.6, 1.8 Hz, 1H), 6.68 (d, J=8.4 Hz, 1H), 4.76 (s, 2H), 2.95 (s, 1H), 2.88 (s, 1H), 2.32 (s, 1H), 1.85-1.70 (m, 4H), 1.10 (t, J=7.4 Hz, 6H).

### Synthesis of 54-4: 2,2-diethyl-4-oxo-1,3-dihydroquinoline-6-carboxylic acid

A mixture of compound 54-3 (300 mg, 1.31 mmol) in concentrated hydrochloric acid solution (1 mL) and acetic acid (1 mL) was stirred at 115° C. for 3 hours. The mixture was basified with 1 N sodium hydroxide solution to pH=10, washed with EtOAc (20 mL×3), the aqueous phase was acidized with 1 N hydrochloric acid solution to pH=3, filtered, the filtrate cake was washed with water (10 mL), dried under vacuum to give compound 54-4 (35 mg, yield: 11%) as white solid.

¹H NMR (400 MHz, DMSO-d₆) δ=12.08 (br. s, 1H), 7.90 (d, J=2.0 Hz, 1H), 7.52 (dd, J=2.0, 8.7 Hz, 1H), 7.14 (s, 1H), 6.58 (d, J=8.7 Hz, 1H), 1.73 (s, 1H), 1.36-1.19 (m, 4H), 0.58 (t, J=7.4 Hz, 6H).

### Synthesis of 55-1: 2,2-diethyl-6-[3-(thiophen-3-yl)-1,2,4-oxadiazol-5-yl]-1,3-dihydroquinolin-4-one

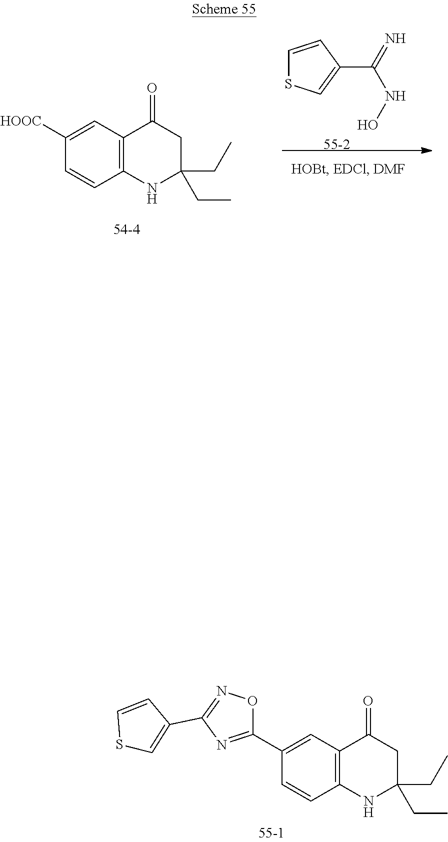 [SMILES: CCC1(CC)CC(=O)c2cc(C(=O)O)ccc2N1 ; N=C(NO)c1ccsc1 ; CCC1(CC)CC(=O)c2cc(-c3nc(-c4ccsc4)no3)ccc2N1]

To a solution of compound 54-4 (100 mg, 404.39 umol, 1 eq.) in DMF (1 mL) was added HOBt (65.57 mg, 485.26 umol, 1.2 eq.) and EDCI (93.02 mg, 485.26 umol, 1.2 eq.). After stirring at 20° C. for 30 mins, compound 55-2 (63.24 mg, 444.82 umol, 1.1 eq.) was added, and stirred for another 30 mins. Then the mixture was heated to 120° C. and stirred for 2 hours. The mixture was triturated with EA (20 mL), filtered, washed with EA (10 mL), the residue was purified by prep-HPLC (column: Phenomenex Synergi C18 150×25×10 um; mobile phase: [water (0.1% TFA)-ACN]; B %: 50%-80%, 13 min) to give 55-1 (45 mg, yield: 29%) as orange solid. ¹H NMR (400 MHz, DMSO-d₆) δ=8.36-8.27 (m, 2H), 7.96 (dd, J=2.0, 8.8 Hz, 1H), 7.79 (dd, J=3.0, 5.0 Hz, 1H), 7.71 (s, 1H), 7.62 (d, J=5.0 Hz, 1H), 6.99 (d, J=8.9 Hz, 1H), 2.59 (s, 2H), 1.67-1.48 (m, 4H), 0.86 (t, J=7.3 Hz, 6H).

### General Procedure O

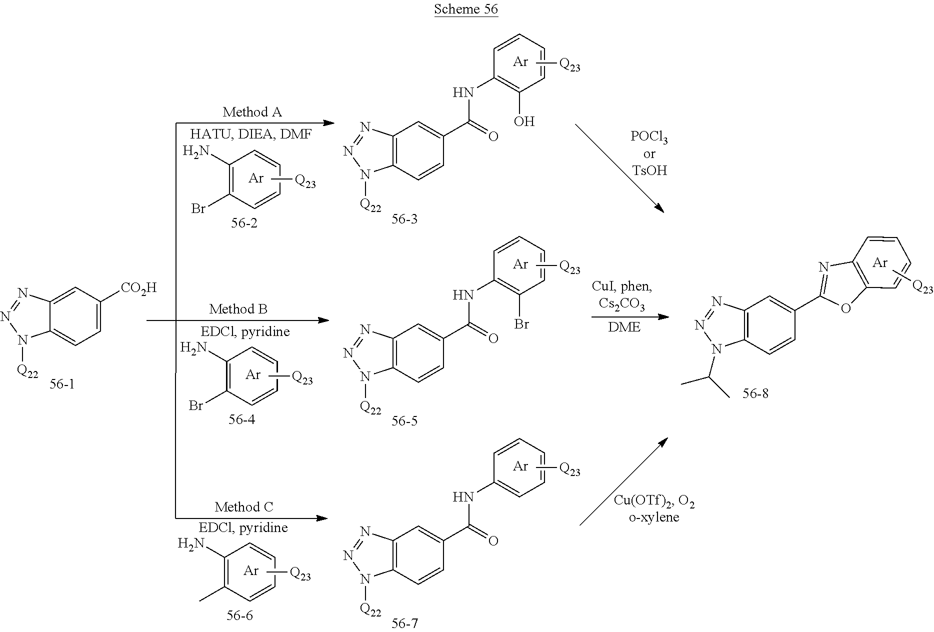

### Synthesis of 57-3: 5-(4-methoxy-1,3-benzoxazol-2-yl)-1-(2-methylpropyl)-1,2,3-benzotriazole

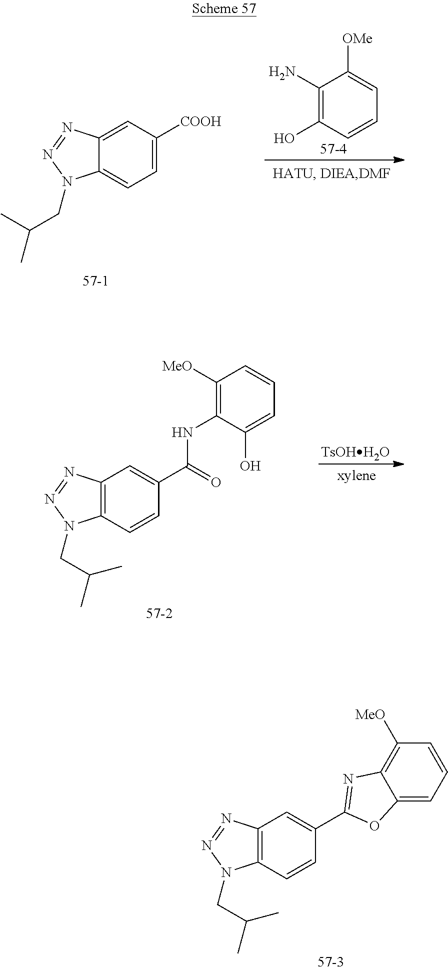

### Synthesis of 57-2: N-(2-hydroxy-6-methoxyphenyl)-1-(2-methylpropyl)-1,2,3-benzotriazole-5-carboxamide

To a solution of compound 57-1 (100 mg, 487.30 umol, 1 eq.) in DMF (3 mL) was added HOBt (65.85 mg, 487.30 umol, 1 eq.) and EDCI (112.10 mg, 584.76 umol, 1.2 eq.). After addition, the mixture was stirred at 20° C. for 0.5 hour, then compound 57-4 (81.37 mg, 584.76 umol, 1.2 eq.) was added, the mixture was stirred at 20° C. for further 12 hours. LCMS showed compound 57-1 consumed, and a major peak with desired MS detected. The mixture was diluted with water (20 mL), extracted with EtOAc (15 mL*2), washed with brine (20 mL), dried with sodium sulfate, filtered and concentrated, to give compound 57-2 (159 mg, crude) as brown oil, which was used in next step directly without further purification. LCMS: 327.2[M+1]

### Synthesis of 57-3: 5-(4-methoxy-1,3-benzoxazol-2-yl)-1-(2-methylpropyl)-1,2,3-benzotriazole

To a solution of compound 57-2 (159 mg, crude) in xylene (10 mL) was added TsOH.H₂O (370.70 mg, 1.95 mmol, 4 eq.). After addition, the mixture was stirred at 120° C. for 2 hours. LCMS showed compound 57-2 consumed, and a major peak with desired Ms detected. The mixture was concentrated, diluted with saturated sodium bicarbonate solution (20 mL), extracted with EtOAc (20 mL*2), dried with sodium sulfate, filtered and concentrated. The residue was purified by prep-HPLC (column: Phenomenex Synergi C18 150*25*10 um; mobile phase: [water (0.1% TFA)-ACN]; B %: 42%-72%, 10 min) to give 57-3 (53 mg, yield: 35%) as grey solids.

¹H NMR (400 MHz, CDCl₃) δ=8.95 (s, 1H), 8.51 (dd, J=1.1, 8.8 Hz, 1H), 7.69 (d, J=8.8 Hz, 1H), 7.37-7.31 (m, 1H), 7.28-7.27 (m, 1H), 6.85 (d, J=7.9 Hz, 1H), 5.18-5.08 (m, 1H), 4.10 (s, 3H), 1.80 (s, 3H), 1.78 (s, 3H)

### Synthesis of 58-3: 1-isopropyl-5-(7-methoxy-1,3-benzoxazol-2-yl)-1,2,3-benzotriazole

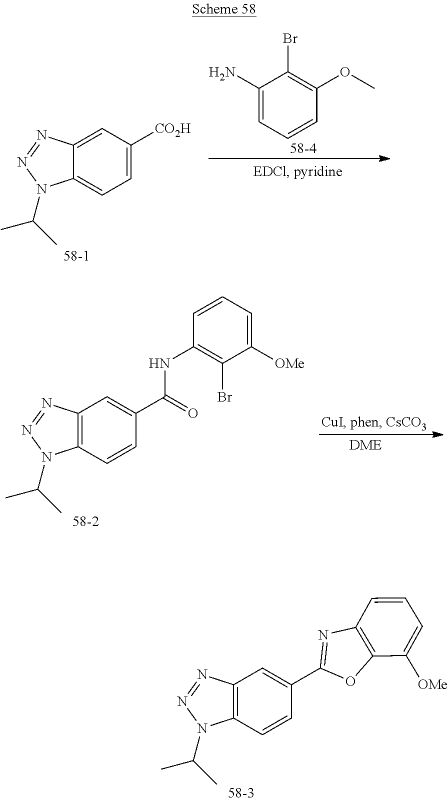

### Synthesis of 58-2: N-(2-bromo-3-methoxyphenyl)-1-isopropyl-1,2,3-benzotriazole-5-carboxamide

A mixture of compound 58-1 (200 mg, 989.86 umol, 1 eq.), compound 58-1 (243.76 mg, 1.19 mmol, 1.2 eq.) and EDCI (284.64 mg, 1.48 mmol, 1.5 eq.) in pyridine (3 mL) was stirred at 20° C. for 12 hours. The mixture was diluted with EtOAc (30 mL), washed with 1 N hydrochloric acid solution (20 mL*3), dried with sodium sulfate, filtered and concentrated. The residue was purified by prep-TLC (PE:EA=3:1) to give compound 58-2 (150 mg, yield: 38%) as brown oil. LCMS: 391.1 [M+1]

### Synthesis of 58-3: 1-isopropyl-5-(7-methoxy-1,3-benzoxazol-2-yl)-1,2,3-benzotriazole

A mixture of compound 58-2 (50 mg, 128.45 umol, 1 eq.), 1,10-phenanthroline (2.31 mg, 12.85 umol, 0.1 eq.), Cs₂CO₃ (62.78 mg, 192.68 umol, 1.5 eq.) and CuI (1.22 mg, 6.42 umol, 0.05 eq.) in DME (2 mL) was heated to 85° C. and stirred for 12 hours under nitrogen atmosphere. The mixture was diluted with EtOAc (20 mL), washed with water (10 mL), dried with sodium sulfate, filtered and concentrated. The residue was purified by prep-HPLC (column: Phenomenex Synergi C18 150*25*10 um; mobile phase: [water (0.1% TFA)-ACN]; B %: 45%-75%, 12 min) to give 58-3 (8 mg, yield: 19%) as gray solid.

¹H NMR (400 MHz, DMSO-d) δ=8.76 (s, 1H), 8.32 (dd, J=1.3, 8.8 Hz, 1H), 8.13 (d, J=8.7 Hz, 1H), 7.44-7.28 (m, 2H), 7.07 (d, J=7.8 Hz, 1H), 5.29 (spt, J=6.7 Hz, 1H), 4.02 (s, 3H), 1.66 (d, J=6.6 Hz, 6H).

### Synthesis of 59-3: 1-isopropyl-5-(6-methoxy-4-methyl-1,3-benzoxazol-2-yl)-1,2,3-benzotriazole

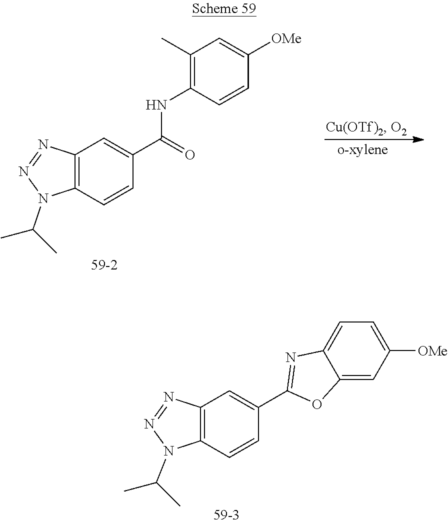 [SMILES: COc1ccc(NC(=O)c2ccc3c(c2)nnn3C(C)C)c(C)c1 ; COc1ccc2nc(-c3ccc4c(c3)nnn4C(C)C)oc2c1]

### Synthesis of 59-3: 1-isopropyl-5-(6-methoxy-4-methyl-1,3-benzoxazol-2-yl)-1,2,3-benzotriazole

To a solution of compound 59-2 (1-isopropyl-N-(4-methoxy-2-methylphenyl)-1,2,3-benzotriazole-5-carboxamide) prepared according to the procedure to prepare 58-2, (50 mg, 154.14 umol, 1 eq.) in o-xylene (2 mL) was added Cu(OTf)₂ (11.12 mg, 30.83 umol, 0.2 eq.). The reaction was stirred at 130° C. under oxygen atmosphere for 16 hours. The mixture was diluted with water (20 mL), extracted with EtOAc (30 mL*3), dried with sodium sulfate, filtered and concentrated. The residue was purified by prep-TLC (PE:EA=4:1) to give 59-3 (1.1 mg, yield: 2%) as yellow solid.

¹H NMR (400 MHz, CDCl₃) δ=8.80 (s, 1H), 8.33 (dd, J=1.4, 8.7 Hz, 1H), 7.59 (d, J=8.8 Hz, 1H), 6.91 (d, J=2.2 Hz, 1H), 6.72 (d, J=1.5 Hz, 1H), 5.16-4.93 (m, 1H), 3.81 (s, 3H), 2.58 (s, 3H), 1.72 (d, J=6.8 Hz, 6H)

### Example 2: In Vitro Activity of Compounds

Stable clonal Chinese hamster ovary K1 (CHO-K1) cells co-expressing EA-β-arrestin2 and the human sphingosine-1-phosphate receptor 1 (NM_001400, S1P1) with a C-terminal Prolink™ tag were purchased from DiscoverX corporation (Cat #: 93-0207C2).

### Cell Culturing and Assay Plating

Cell lines were cultured in AssayComplete™ Media 6 (DiscoverX Corporation, Cat #: 920018GF2) at 37° C. and 5% CO₂ in a humidified CO₂ and temperature-controlled incubator. To begin assay plating, cells were washed with Dulbecco's phosphate buffered saline (CellGro, Cat #: 21-031-CV) and lifted from the culturing flask by incubation (37° C., 5 min) with CellStripper (Cellgro, Cat #: 25-056-CI). Lifted cells were resuspended to 250,000 cells per milliliter in AssayComplete™ Cell Plating 11 Reagent (DiscoverX Corporation, Cat #: 93-0563R11B) and plated at 5,000 cells per well in white-opaque 384 well plates (Greiner Bio-One Item #: 20-784080). Plated cells were incubated overnight at 37° C. and 5% CO₂ in a humidified CO₂ and temperature-controlled incubator.

Detecting Inhibition of cAMP Production

Agonist-promoted G-protein responses were determined by measuring changes in intracellular cAMP using the HTRF® cAMP HiRange kit (CisBio, Cat #: 62AM6PEJ) based on time-resolved fluorescence resonance energy transfer (TR-FRET) technology. AssayComplete™ Cell Plating 11 Reagent was removed and replaced with Ham's F-12 (CellGro, Cat #: 10-080-CM) containing isobutyl-methyl-xanthine (IBMX; 500 μM; Tocris Bioscience, Cat #: 2845), and NKH-477 (1.5 μM; Tocris Bioscience, Cat #: 1603) along with test or reference compound at the desired concentrations. Following a 30-minute incubation at 37° C. and 5% CO₂ in a humidified CO₂ and temperature-controlled incubator, the components of the cAMP HiRange kit were added as per the manufacturer's instructions. After an hour incubation at room temperature, plates were analyzed by a BMG PheraStar microplate reader. Responses were measured as the ratio of fluorescence emission at 665 nm to fluorescence emission at 620 nm.

### β-Arrestin2 Recruitment Assay

Agonist-promoted β-arrestin2 recruitment to the sphingosine-1-phosphate 1 receptor was determined using the β-arrestin PathHunter® Detection kit (DiscoverX Corporation, Cat #: 93-0001). In this system, β-arrestin2 is fused to an N-terminal deletion mutant of β-galactosidase (termed the enzyme acceptor or EA) and the C-terminus of the GPCR of interest is fused to a smaller (42 amino acids), weakly-complementing fragment 15 termed ProLink™. In cells that stably express these fusion proteins, stimulation with a cognate agonist results in the interaction of β-arrestin2 and the Prolink™-tagged GPCR. This allows the complementation of the two β-galactosidase fragments and results in the formation of a functional enzyme with β-galactosidase activity. AssayComplete™ Cell Plating 11 Reagent was removed and replaced with Ham's F-12 containing IBMX (500 μM), and NKH-477 (1.5 μM) along with test or reference compounds at the desired concentrations. Following a 60 minute incubation at 37° C. and 5% CO₂ in a humidified CO₂ and temperature-controlled incubator, the components of the β-arrestin PathHunter® Detection kit were added as per the manufacturer's instructions. After an hour incubation at room temperature, plates were analyzed by a BMG PheraStar microplate reader.

### Activity Table

The compounds as indicated herein were able to modulate the activities (inhibition of cAMP production and β-arrestin2 recruitment) of the sphingosine-1-phosphate 1 receptor as indicated herein. The tables below include the efficacy of the compound normalized to the maximum efficacy of a reference compound, referred to as “SPAN”. These values are normalized to fingolimod, a known agonist of the sphingosine-1-phosphate 1 receptor. The tables also include potency values (pEC50) for modulating discrete receptor-mediated activities (inhibition of cAMP production and β-arrestin2 recruitment). This value represents the estimated concentration to promote half of the maximal efficacy (or SPAN) observed for each compound.

| | hS1P1 | hS1P1 | hS1P1 | hS1P1 |
|---|---|---|---|---|
| Compound | CAMP | CAMP | B-arrestin2 | B-arrestin2 |
| Number | pEC₅₀ | SPAN | pEC₅₀ | SPAN |
| 1 | <6.5 | <75 | <5 | <75 |
| 2 | <6.5 | >75 | >5 | <75 |
| 3 | >6.5 | >75 | >5 | >75 |
| 4 | <6.5 | <75 | <5 | <75 |
| 5 | <6.5 | <75 | <5 | <75 |
| 6 | <6.5 | <75 | <5 | <75 |
| 7 | <6.5 | >75 | <5 | >75 |
| 8 | <6.5 | <75 | <5 | <75 |
| 9 | <6.5 | <75 | <5 | <75 |
| 10 | <6.5 | <75 | <5 | <75 |
| 11 | <6.5 | <75 | <5 | <75 |
| 12 | <6.5 | <75 | <5 | <75 |
| 13 | >6.5 | <75 | >5 | <75 |
| 14 | >6.5 | <75 | >5 | <75 |
| 15 | <6.5 | >75 | <5 | <75 |
| 16 | <6.5 | <75 | >5 | >75 |
| 17 | >6.5 | >75 | >5 | >75 |
| 18 | <6.5 | <75 | <5 | <75 |
| 19 | <6.5 | <75 | >5 | <75 |
| 20 | >6.5 | >75 | >5 | >75 |
| 21 | <6.5 | >75 | <5 | >75 |
| 22 | >6.5 | >75 | >5 | >75 |
| 23 | <6.5 | <75 | <5 | <75 |
| 24 | <6.5 | >75 | <5 | <75 |
| 25 | <6.5 | <75 | <5 | <75 |
| 26 | >6.5 | >75 | >5 | >75 |
| 26 | >6.5 | >75 | >5 | <75 |
| 27 | <6.5 | >75 | >5 | <75 |
| 28 | <6.5 | >75 | <5 | >75 |
| 29 | <6.5 | <75 | <5 | <75 |
| 30 | <6.5 | <75 | <5 | <75 |
| 31 | >6.5 | >75 | >5 | >75 |
| 32 | >6.5 | >75 | >5 | <75 |
| 33 | >6.5 | >75 | >5 | >75 |
| 34 | <6.5 | >75 | >5 | <75 |
| 35 | <6.5 | >75 | <5 | >75 |
| 36 | <6.5 | <75 | <5 | <75 |
| 37 | <6.5 | <75 | <5 | <75 |
| 38 | <6.5 | <75 | <5 | >75 |
| 39 | <6.5 | <75 | <5 | <75 |
| 40 | >6.5 | >75 | >5 | >75 |
| 41 | <6.5 | <75 | <5 | <75 |
| 41 | <6.5 | <75 | <5 | <75 |
| 42 | <6.5 | >75 | <5 | >75 |
| 43 | >6.5 | >75 | >5 | >75 |
| 44 | <6.5 | <75 | <5 | <75 |
| 45 | <6.5 | <75 | <5 | <75 |
| 46 | >6.5 | >75 | >5 | >75 |
| 47 | <6.5 | <75 | <5 | <75 |
| 48 | <6.5 | <75 | <5 | <75 |
| 49 | <6.5 | <75 | <5 | <75 |
| 50 | <6.5 | <75 | <5 | <75 |
| 51 | <6.5 | <75 | <5 | <75 |
| 52 | <6.5 | <75 | <5 | <75 |
| 53 | <6.5 | <75 | <5 | <75 |
| 54 | <6.5 | <75 | <5 | >75 |
| 55 | >6.5 | >75 | >5 | >75 |
| 56 | <6.5 | <75 | <5 | <75 |
| 57 | <6.5 | <75 | <5 | <75 |
| 58 | <6.5 | <75 | <5 | <75 |
| 59 | >6.5 | >75 | >5 | >75 |
| 60 | <6.5 | <75 | <5 | <75 |
| 61 | >6.5 | >75 | >5 | >75 |
| 62 | <6.5 | >75 | <5 | >75 |
| 63 | >6.5 | <75 | <5 | <75 |
| 64 | >6.5 | >75 | <5 | >75 |
| 65 | <6.5 | <75 | <5 | <75 |
| 66 | >6.5 | >75 | >5 | >75 |
| 67 | <6.5 | >75 | >5 | <75 |
| 68 | <6.5 | <75 | <5 | <75 |
| 69 | <6.5 | <75 | >5 | <75 |
| 70 | >6.5 | <75 | >5 | <75 |
| 71 | <6.5 | >75 | <5 | <75 |
| 72 | >6.5 | >75 | >5 | >75 |
| 73 | <6.5 | <75 | >5 | <75 |
| 74 | <6.5 | <75 | <5 | <75 |
| 75 | >6.5 | <75 | >5 | >75 |
| 76 | <6.5 | <75 | <5 | <75 |
| 77 | <6.5 | <75 | <5 | <75 |
| 78 | <6.5 | <75 | <5 | <75 |
| 79 | >6.5 | >75 | >5 | >75 |
| 80 | >6.5 | <75 | <5 | <75 |
| 81 | <6.5 | >75 | <5 | <75 |
| 82 | <6.5 | <75 | <5 | <75 |
| 83 | >6.5 | >75 | >5 | >75 |
| 84 | <6.5 | <75 | >5 | >75 |
| 85 | <6.5 | <75 | <5 | <75 |
| 86 | <6.5 | <75 | <5 | >75 |
| 87 | <6.5 | <75 | <5 | <75 |
| 88 | <6.5 | <75 | <5 | <75 |
| 89 | <6.5 | <75 | <5 | <75 |
| 90 | <6.5 | >75 | >5 | >75 |
| 91 | <6.5 | <75 | <5 | <75 |
| 92 | <6.5 | <75 | <5 | <75 |
| 93 | <6.5 | <75 | <5 | >75 |
| 94 | <6.5 | >75 | <5 | >75 |
| 95 | <6.5 | <75 | <5 | <75 |
| 96 | >6.5 | >75 | >5 | >75 |
| 97 | <6.5 | <75 | <5 | <75 |
| 98 | <6.5 | >75 | >5 | >75 |
| 99 | >6.5 | >75 | >5 | >75 |
| 100 | <6.5 | <75 | <5 | <75 |
| 101 | >6.5 | >75 | >5 | >75 |
| 102 | <6.5 | <75 | <5 | <75 |
| 103 | >6.5 | >75 | >5 | >75 |
| 103 | >6.5 | >75 | >5 | >75 |
| 103 | >6.5 | >75 | >5 | >75 |
| 104 | >6.5 | >75 | <5 | >75 |
| 105 | <6.5 | <75 | <5 | <75 |
| 106 | >6.5 | >75 | >5 | >75 |
| 107 | >6.5 | >75 | >5 | >75 |
| 108 | >6.5 | <75 | <5 | <75 |
| 109 | <6.5 | <75 | >5 | <75 |
| 110 | <6.5 | <75 | <5 | <75 |
| 111 | <6.5 | >75 | <5 | >75 |
| 112 | >6.5 | >75 | >5 | >75 |
| 113 | <6.5 | <75 | <5 | <75 |
| 114 | <6.5 | <75 | <5 | <75 |
| 115 | >6.5 | >75 | >5 | >75 |
| 116 | <6.5 | <75 | <5 | <75 |
| 117 | <6.5 | <75 | <5 | <75 |
| 118 | <6.5 | <75 | <5 | <75 |
| 119 | <6.5 | <75 | <5 | <75 |
| 120 | >6.5 | >75 | >5 | >75 |
| 121 | <6.5 | <75 | <5 | <75 |
| 122 | <6.5 | <75 | <5 | <75 |
| 123 | >6.5 | >75 | >5 | >75 |
| 124 | <6.5 | <75 | <5 | <75 |
| 125 | <6.5 | <75 | <5 | <75 |
| 126 | <6.5 | <75 | <5 | <75 |
| 127 | <6.5 | <75 | <5 | <75 |
| 128 | <6.5 | <75 | <5 | <75 |
| 129 | <6.5 | <75 | <5 | <75 |
| 130 | <6.5 | <75 | <5 | <75 |
| 131 | <6.5 | <75 | <5 | <75 |
| 132 | >6.5 | >75 | >5 | >75 |
| 133 | <6.5 | <75 | <5 | <75 |
| 134 | <6.5 | <75 | <5 | <75 |
| 135 | <6.5 | <75 | <5 | <75 |
| 136 | <6.5 | <75 | <5 | <75 |
| 137 | >6.5 | >75 | <5 | >75 |
| 138 | >6.5 | >75 | >5 | >75 |
| 139 | >6.5 | >75 | >5 | >75 |
| 140 | >6.5 | >75 | >5 | >75 |
| 141 | >6.5 | >75 | >5 | >75 |
| 142 | >6.5 | >75 | >5 | >75 |
| 143 | <6.5 | <75 | <5 | <75 |
| 144 | <6.5 | >75 | >5 | <75 |
| 145 | >6.5 | >75 | >5 | <75 |
| 146 | >6.5 | >75 | >5 | <75 |
| 147 | >6.5 | >75 | >5 | >75 |
| 148 | <6.5 | <75 | <5 | <75 |
| 149 | >6.5 | >75 | >5 | >75 |
| 150 | >6.5 | >75 | >5 | >75 |
| 151 | <6.5 | >75 | <5 | >75 |
| 152 | <6.5 | >75 | >5 | >75 |
| 153 | <6.5 | >75 | >5 | <75 |
| 154 | <6.5 | <75 | <5 | <75 |
| 155 | <6.5 | <75 | <5 | <75 |
| 156 | >6.5 | >75 | >5 | >75 |
| 157 | <6.5 | <75 | <5 | <75 |
| 158 | <6.5 | <75 | <5 | <75 |
| 159 | <6.5 | <75 | <5 | <75 |
| 160 | <6.5 | <75 | <5 | <75 |
| 161 | <6.5 | <75 | <5 | >75 |
| 162 | <6.5 | >75 | >5 | >75 |
| 163 | >6.5 | >75 | >5 | >75 |
| 164 | <6.5 | <75 | <5 | <75 |
| 165 | <6.5 | >75 | <5 | >75 |
| 166 | <6.5 | <75 | <5 | >75 |
| 167 | <6.5 | <75 | <5 | <75 |
| 168 | <6.5 | <75 | <5 | <75 |
| 169 | >6.5 | >75 | >5 | >75 |
| 170 | <6.5 | <75 | <5 | <75 |
| 171 | >6.5 | >75 | >5 | >75 |
| 172 | <6.5 | <75 | <5 | <75 |
| 173 | >6.5 | >75 | >5 | >75 |
| 174 | >6.5 | >75 | <5 | >75 |
| 175 | >6.5 | >75 | >5 | >75 |
| 176 | <6.5 | >75 | <5 | <75 |
| 177 | >6.5 | >75 | >5 | >75 |
| 178 | <6.5 | >75 | >5 | <75 |
| 179 | >6.5 | >75 | >5 | >75 |
| 180 | <6.5 | >75 | <5 | >75 |
| 181 | <6.5 | >75 | <5 | >75 |
| 182 | >6.5 | >75 | >5 | <75 |
| 183 | >6.5 | >75 | >5 | >75 |
| 184 | <6.5 | >75 | <5 | <75 |
| 185 | >6.5 | >75 | >5 | >75 |
| 186 | <6.5 | <75 | <5 | <75 |
| 187 | <6.5 | <75 | <5 | >75 |
| 188 | <6.5 | <75 | <5 | <75 |
| 189 | >6.5 | >75 | >5 | >75 |
| 190 | <6.5 | <75 | <5 | <75 |
| 192 | <6.5 | <75 | <5 | <75 |
| 193 | <6.5 | <75 | <5 | <75 |
| 194 | <6.5 | <75 | <5 | <75 |
| 195 | <6.5 | <75 | <5 | <75 |
| 196 | <6.5 | <75 | <5 | <75 |
| 197 | <6.5 | <75 | <5 | <75 |
| 198 | >6.5 | >75 | >5 | <75 |
| 199 | <6.5 | <75 | <5 | <75 |
| 200 | <6.5 | <75 | <5 | <75 |
| 201 | <6.5 | <75 | <5 | >75 |
| 202 | <6.5 | <75 | <5 | <75 |
| 203 | <6.5 | <75 | <5 | >75 |
| 204 | <6.5 | >75 | >5 | >75 |
| 205 | <6.5 | >75 | <5 | <75 |
| 206 | <6.5 | >75 | <5 | <75 |
| 207 | >6.5 | >75 | >5 | >75 |
| 208 | >6.5 | >75 | >5 | >75 |
| 209 | >6.5 | >75 | >5 | >75 |
| 210 | >6.5 | >75 | >5 | >75 |
| 211 | <6.5 | <75 | >5 | >75 |
| 212 | >6.5 | >75 | >5 | <75 |
| 213 | <6.5 | >75 | <5 | >75 |
| 214 | <6.5 | <75 | <5 | <75 |
| 215 | <6.5 | <75 | <5 | <75 |
| 216 | <6.5 | <75 | <5 | <75 |
| 217 | <6.5 | <75 | <5 | <75 |
| 218 | <6.5 | >75 | <5 | >75 |
| 219 | >6.5 | <75 | >5 | >75 |
| 220 | <6.5 | <75 | <5 | <75 |
| 221 | >6.5 | >75 | >5 | >75 |
| 222 | <6.5 | >75 | >5 | <75 |
| 223 | <6.5 | <75 | <5 | <75 |
| 224 | >6.5 | >75 | >5 | >75 |
| 225 | <6.5 | >75 | >5 | <75 |
| 226 | <6.5 | >75 | <5 | >75 |
| 227 | >6.5 | <75 | <5 | >75 |
| 228 | <6.5 | <75 | <5 | <75 |
| 229 | <6.5 | <75 | >5 | <75 |
| 230 | <6.5 | <75 | <5 | <75 |
| 231 | <6.5 | <75 | <5 | <75 |
| 232 | <6.5 | <75 | <5 | <75 |
| 233 | <6.5 | <75 | <5 | <75 |
| 234 | <6.5 | <75 | <5 | <75 |
| 235 | <6.5 | <75 | <5 | <75 |
| 236 | <6.5 | <75 | <5 | >75 |
| 237 | <6.5 | <75 | <5 | <75 |
| 238 | <6.5 | <75 | <5 | <75 |
| 239 | <6.5 | <75 | <5 | <75 |
| 240 | <6.5 | >75 | <5 | >75 |
| 241 | <6.5 | >75 | <5 | >75 |
| 242 | <6.5 | <75 | <5 | <75 |
| 243 | <6.5 | >75 | <5 | >75 |
| 244 | >6.5 | >75 | >5 | >75 |
| 245 | <6.5 | >75 | >5 | >75 |
| 246 | >6.5 | >75 | >5 | >75 |
| 247 | <6.5 | <75 | <5 | <75 |
| 248 | <6.5 | <75 | <5 | <75 |
| 249 | <6.5 | >75 | <5 | <75 |
| 250 | <6.5 | <75 | <5 | <75 |
| 251 | <6.5 | <75 | <5 | <75 |
| 252 | >6.5 | >75 | >5 | >75 |
| 253 | >6.5 | >75 | >5 | >75 |
| 254 | >6.5 | <75 | <5 | <75 |
| 255 | <6.5 | <75 | <5 | <75 |
| 256 | >6.5 | >75 | >5 | >75 |
| 257 | <6.5 | >75 | <5 | <75 |
| 258 | <6.5 | <75 | <5 | <75 |
| 259 | >6.5 | >75 | >5 | <75 |
| 260 | <6.5 | >75 | <5 | >75 |
| 261 | <6.5 | >75 | >5 | <75 |
| 262 | >6.5 | >75 | >5 | >75 |
| 263 | >6.5 | >75 | >5 | >75 |
| 264 | <6.5 | <75 | <5 | <75 |
| 265 | >6.5 | >75 | >5 | <75 |
| 266 | <6.5 | >75 | >5 | >75 |
| 267 | >6.5 | <75 | >5 | <75 |
| 268 | >6.5 | >75 | >5 | >75 |
| 269 | <6.5 | <75 | <5 | <75 |
| 270 | <6.5 | <75 | <5 | <75 |
| 271 | <6.5 | <75 | <5 | <75 |
| 272 | <6.5 | <75 | <5 | <75 |
| 273 | <6.5 | <75 | <5 | <75 |
| 274 | <6.5 | >75 | >5 | <75 |
| 275 | <6.5 | <75 | <5 | <75 |
| 276 | >6.5 | >75 | >5 | >75 |
| 277 | >6.5 | >75 | >5 | >75 |
| 278 | <6.5 | <75 | <5 | <75 |
| 279 | <6.5 | >75 | <5 | >75 |
| 280 | <6.5 | <75 | <5 | <75 |
| 281 | <6.5 | <75 | <5 | <75 |
| 282 | >6.5 | >75 | >5 | >75 |
| 283 | <6.5 | <75 | <5 | <75 |
| 284 | <6.5 | <75 | <5 | <75 |
| 285 | <6.5 | <75 | <5 | <75 |
| 286 | >6.5 | >75 | >5 | >75 |
| 287 | >6.5 | >75 | >5 | >75 |
| 288 | <6.5 | <75 | <5 | <75 |
| 289 | <6.5 | <75 | <5 | <75 |
| 290 | <6.5 | <75 | <5 | <75 |
| 291 | <6.5 | <75 | <5 | <75 |
| 292 | <6.5 | <75 | <5 | <75 |
| 293 | >6.5 | >75 | >5 | >75 |
| 294 | <6.5 | <75 | <5 | >75 |
| 295 | <6.5 | >75 | <5 | <75 |
| 296 | <6.5 | <75 | <5 | <75 |
| 297 | <6.5 | >75 | <5 | >75 |
| 298 | <6.5 | >75 | >5 | <75 |
| 299 | <6.5 | <75 | <5 | >75 |
| 300 | <6.5 | >75 | <5 | >75 |
| 301 | <6.5 | <75 | <5 | <75 |
| 302 | >6.5 | >75 | >5 | >75 |
| 303 | <6.5 | >75 | <5 | >75 |
| 304 | >6.5 | <75 | >5 | >75 |
| 305 | <6.5 | >75 | <5 | <75 |
| 306 | >6.5 | >75 | >5 | <75 |
| 307 | >6.5 | >75 | >5 | >75 |
| 308 | <6.5 | <75 | <5 | <75 |
| 309 | <6.5 | >75 | <5 | >75 |
| 310 | >6.5 | >75 | >5 | >75 |
| 311 | <6.5 | >75 | <5 | >75 |
| 312 | <6.5 | >75 | <5 | >75 |
| 313 | >6.5 | >75 | <5 | >75 |
| 314 | <6.5 | <75 | <5 | <75 |
| 315 | >6.5 | >75 | >5 | <75 |
| 316 | >6.5 | >75 | <5 | <75 |
| 320 | >6.5 | >75 | >5 | >75 |
| 321 | <6.5 | >75 | >5 | <75 |
| 322 | >6.5 | >75 | >5 | >75 |
| 323 | <6.5 | >75 | <5 | >75 |
| 324 | <6.5 | <75 | <5 | <75 |
| 325 | <6.5 | <75 | <5 | <75 |
| 326 | <6.5 | >75 | >5 | >75 |
| 327 | <6.5 | <75 | >5 | >75 |
| 328 | <6.5 | >75 | >5 | >75 |
| 329 | >6.5 | >75 | >5 | >75 |
| 330 | >6.5 | >75 | >5 | >75 |
| 331 | >6.5 | >75 | >5 | >75 |
| 332 | >6.5 | >75 | >5 | >75 |
| 333 | >6.5 | >75 | <5 | >75 |
| 334 | <6.5 | >75 | <5 | >75 |
| 335 | <6.5 | <75 | <5 | <75 |
| 336 | >6.5 | >75 | >5 | <75 |
| 337 | >6.5 | >75 | >5 | >75 |
| 338 | <6.5 | >75 | <5 | >75 |
| 339 | <6.5 | <75 | <5 | <75 |
| 340 | <6.5 | <75 | <5 | <75 |
| 341 | <6.5 | <75 | <5 | <75 |
| 342 | <6.5 | <75 | <5 | <75 |
| 343 | >6.5 | >75 | >5 | >75 |
| 344 | >6.5 | >75 | >5 | >75 |
| 345 | <6.5 | <75 | <5 | <75 |
| 346 | >6.5 | >75 | >5 | >75 |
| 347 | >6.5 | >75 | >5 | >75 |
| 348 | >6.5 | >75 | >5 | >75 |
| 349 | >6.5 | <75 | <5 | <75 |
| 350 | <6.5 | <75 | <5 | <75 |
| 351 | >6.5 | >75 | >5 | >75 |
| 352 | >6.5 | >75 | >5 | >75 |
| 353 | >6.5 | >75 | >5 | >75 |
| 354 | >6.5 | >75 | >5 | >75 |
| 355 | >6.5 | >75 | >5 | >75 |
| 356 | >6.5 | >75 | >5 | >75 |
| 357 | >6.5 | >75 | >5 | >75 |
| 358 | >6.5 | >75 | >5 | >75 |
| 359 | >6.5 | >75 | >5 | >75 |
| 360 | <6.5 | <75 | <5 | <75 |
| 361 | <6.5 | <75 | <5 | <75 |
| 362 | <6.5 | <75 | <5 | <75 |
| 363 | <6.5 | <75 | <5 | <75 |
| 364 | <6.5 | >75 | <5 | <75 |
| 365 | <6.5 | >75 | >5 | <75 |
| 366 | <6.5 | <75 | <5 | <75 |
| 367 | <6.5 | <75 | <5 | <75 |
| 368 | <6.5 | <75 | <5 | <75 |
| 369 | <6.5 | <75 | <5 | <75 |
| 370 | >6.5 | >75 | >5 | >75 |
| 371 | <6.5 | <75 | <5 | <75 |
| 372 | >6.5 | >75 | >5 | >75 |
| 373 | <6.5 | >75 | <5 | >75 |
| 374 | <6.5 | <75 | <5 | <75 |
| 375 | >6.5 | >75 | >5 | <75 |
| 376 | <6.5 | <75 | <5 | <75 |
| 377 | <6.5 | <75 | <5 | <75 |
| 378 | <6.5 | <75 | <5 | <75 |
| 379 | <6.5 | <75 | <5 | <75 |
| 380 | <6.5 | <75 | <5 | <75 |
| 381 | <6.5 | <75 | >5 | <75 |
| 382 | >6.5 | >75 | >5 | >75 |
| 383 | >6.5 | >75 | >5 | >75 |
| 384 | <6.5 | >75 | <5 | <75 |
| 385 | <6.5 | <75 | <5 | >75 |
| 386 | <6.5 | >75 | <5 | <75 |
| 387 | <6.5 | >75 | >5 | >75 |
| 388 | >6.5 | >75 | >5 | >75 |
| 389 | >6.5 | >75 | >5 | >75 |
| 390 | >6.5 | >75 | >5 | <75 |
| 391 | >6.5 | >75 | >5 | >75 |
| 392 | <6.5 | <75 | <5 | <75 |
| 393 | <6.5 | <75 | <5 | <75 |
| 394 | <6.5 | <75 | >5 | <75 |
| 395 | >6.5 | <75 | >5 | >75 |
| 396 | <6.5 | <75 | <5 | >75 |
| 397 | <6.5 | <75 | <5 | >75 |
| 398 | >6.5 | >75 | >5 | >75 |
| 399 | >6.5 | >75 | >5 | <75 |
| 400 | <6.5 | <75 | <5 | <75 |
| 401 | <6.5 | <75 | <5 | <75 |
| 402 | >6.5 | <75 | <5 | <75 |
| 403 | <6.5 | <75 | <5 | <75 |
| 404 | <6.5 | >75 | <5 | <75 |
| 405 | <6.5 | <75 | <5 | <75 |
| 406 | >6.5 | >75 | >5 | >75 |
| 407 | <6.5 | >75 | <5 | <75 |
| 408 | <6.5 | >75 | <5 | <75 |
| 409 | <6.5 | >75 | <5 | <75 |
| 410 | <6.5 | <75 | <5 | <75 |
| 411 | <6.5 | <75 | <5 | <75 |
| 412 | <6.5 | >75 | <5 | <75 |
| 413 | >6.5 | <75 | >5 | >75 |
| 414 | >6.5 | <75 | <5 | >75 |
| 415 | >6.5 | <75 | <5 | >75 |
| 416 | >6.5 | >75 | >5 | >75 |
| 417 | >6.5 | >75 | >5 | >75 |
| 418 | >6.5 | >75 | >5 | >75 |
| 419 | >6.5 | >75 | >5 | <75 |
| 420 | >6.5 | >75 | <5 | >75 |
| 421 | <6.5 | >75 | <5 | <75 |
| 422 | >6.5 | >75 | >5 | >75 |
| 423 | >6.5 | >75 | >5 | >75 |
| 424 | >6.5 | >75 | <5 | >75 |
| 425 | >6.5 | >75 | >5 | >75 |
| 426 | >6.5 | >75 | <5 | >75 |
| 427 | <6.5 | >75 | <5 | >75 |
| 428 | <6.5 | >75 | <5 | >75 |
| 429 | <6.5 | >75 | <5 | >75 |
| 430 | <6.5 | >75 | <5 | >75 |
| 431 | <6.5 | >75 | <5 | >75 |
| 432 | >6.5 | >75 | >5 | >75 |
| 433 | >6.5 | >75 | >5 | >75 |
| 434 | <6.5 | <75 | <5 | >75 |
| 435 | >6.5 | >75 | >5 | >75 |
| 436 | >6.5 | >75 | <5 | >75 |
| 437 | >6.5 | >75 | >5 | <75 |
| 438 | >6.5 | >75 | >5 | >75 |
| 439 | <6.5 | >75 | <5 | >75 |
| 440 | >6.5 | >75 | >5 | >75 |
| 441 | >6.5 | >75 | >5 | >75 |
| 442 | >6.5 | >75 | >5 | >75 |
| 443 | >6.5 | >75 | >5 | <75 |
| 444 | <6.5 | <75 | >5 | <75 |
| 445 | >6.5 | >75 | >5 | >75 |
| 446 | <6.5 | <75 | <5 | <75 |
| 447 | >6.5 | >75 | >5 | <75 |
| 448 | <6.5 | >75 | <5 | <75 |
| 449 | <6.5 | >75 | <5 | >75 |
| 450 | >6.5 | >75 | >5 | >75 |
| 451 | >6.5 | <75 | >5 | <75 |
| 452 | <6.5 | >75 | >5 | <75 |
| 453 | >6.5 | >75 | >5 | <75 |
| 454 | >6.5 | >75 | >5 | >75 |
| 455 | >6.5 | >75 | >5 | >75 |
| 456 | >6.5 | >75 | <5 | >75 |
| 457 | >6.5 | >75 | <5 | >75 |
| 458 | <6.5 | <75 | <5 | <75 |
| 459 | <6.5 | >75 | <5 | >75 |
| 460 | <6.5 | >75 | <5 | >75 |
| 461 | <6.5 | >75 | <5 | >75 |
| 462 | >6.5 | >75 | <5 | >75 |
| 463 | >6.5 | >75 | >5 | >75 |
| 464 | >6.5 | <75 | <5 | <75 |
| 465 | >6.5 | >75 | >5 | >75 |
| 466 | <6.5 | >75 | <5 | >75 |
| 467 | >6.5 | >75 | >5 | >75 |
| 468 | >6.5 | >75 | >5 | >75 |
| 469 | >6.5 | >75 | >5 | >75 |
| 470 | <6.5 | >75 | <5 | >75 |
| 471 | >6.5 | >75 | >5 | >75 |
| 472 | >6.5 | >75 | >5 | >75 |
| 473 | >6.5 | >75 | >5 | >75 |
| 474 | <6.5 | >75 | <5 | >75 |
| 475 | >6.5 | >75 | >5 | <75 |
| 476 | >6.5 | >75 | >5 | <75 |
| 477 | >6.5 | >75 | >5 | <75 |
| 478 | <6.5 | <75 | <5 | <75 |
| 479 | <6.5 | <75 | <5 | <75 |
| 480 | <6.5 | >75 | <5 | <75 |
| 481 | <6.5 | >75 | >5 | <75 |
| 482 | <6.5 | <75 | <5 | >75 |
| 483 | <6.5 | >75 | <5 | <75 |
| 484 | >6.5 | >75 | >5 | >75 |
| 485 | >6.5 | >75 | <5 | >75 |
| 486 | >6.5 | <75 | <5 | >75 |
| 487 | <6.5 | >75 | >5 | >75 |
| 488 | >6.5 | <75 | >5 | >75 |
| 489 | >6.5 | >75 | >5 | >75 |
| 490 | >6.5 | >75 | >5 | >75 |
| 491 | >6.5 | >75 | >5 | >75 |
| 492 | <6.5 | >75 | <5 | >75 |
| 493 | <6.5 | >75 | <5 | >75 |
| 494 | <6.5 | <75 | >5 | <75 |
| 495 | >6.5 | >75 | >5 | >75 |
| 496 | >6.5 | >75 | >5 | >75 |
| 497 | >6.5 | >75 | >5 | >75 |
| 498 | >6.5 | >75 | <5 | <75 |
| 499 | >6.5 | >75 | >5 | >75 |
| 500 | <6.5 | <75 | <5 | <75 |
| 501 | <6.5 | <75 | <5 | >75 |
| 502 | >6.5 | >75 | >5 | >75 |
| 503 | <6.5 | >75 | <5 | >75 |
| 504 | <6.5 | >75 | <5 | <75 |
| 505 | <6.5 | <75 | <5 | >75 |
| 506 | >6.5 | <75 | >5 | <75 |
| 507 | >6.5 | <75 | <5 | <75 |
| 508 | <6.5 | >75 | <5 | <75 |
| 509 | <6.5 | <75 | <5 | >75 |
| 510 | >6.5 | <75 | >5 | >75 |
| 511 | >6.5 | <75 | >5 | >75 |
| 512 | >6.5 | >75 | >5 | <75 |
| 513 | >6.5 | >75 | >5 | >75 |
| 514 | >6.5 | >75 | >5 | <75 |
| 515 | >6.5 | >75 | >5 | >75 |
| 516 | <6.5 | <75 | >5 | <75 |
| 517 | <6.5 | <75 | >5 | <75 |
| 518 | <6.5 | >75 | <5 | <75 |
| 519 | >6.5 | >75 | >5 | >75 |
| 520 | >6.5 | >75 | >5 | >75 |
| 521 | <6.5 | <75 | <5 | <75 |
| 522 | <6.5 | >75 | <5 | >75 |
| 523 | <6.5 | >75 | <5 | >75 |
| 524 | >6.5 | >75 | >5 | <75 |
| 525 | <6.5 | >75 | <5 | >75 |
| 526 | <6.5 | >75 | <5 | >75 |
| 527 | <6.5 | >75 | <5 | >75 |
| 528 | <6.5 | >75 | <5 | <75 |
| 529 | <6.5 | >75 | >5 | <75 |
| 530 | <6.5 | >75 | <5 | >75 |
| 531 | >6.5 | >75 | >5 | >75 |
| 532 | >6.5 | >75 | >5 | <75 |
| 533 | <6.5 | <75 | <5 | <75 |
| 534 | <6.5 | <75 | <5 | <75 |
| 535 | <6.5 | <75 | >5 | <75 |
| 536 | <6.5 | <75 | >5 | <75 |
| 537 | >6.5 | >75 | >5 | <75 |
| 538 | >6.5 | <75 | >5 | <75 |
| 539 | >6.5 | <75 | >5 | <75 |
| 540 | <6.5 | <75 | <5 | <75 |
| 541 | <6.5 | <75 | <5 | <75 |
| 542 | <6.5 | <75 | >5 | <75 |
| 543 | >6.5 | <75 | <5 | <75 |
| 544 | <6.5 | <75 | >5 | <75 |
| 545 | >6.5 | >75 | >5 | >75 |
| 546 | <6.5 | <75 | <5 | <75 |
| 547 | >6.5 | >75 | >5 | >75 |
| 548 | <6.5 | <75 | <5 | <75 |
| 549 | >6.5 | >75 | >5 | >75 |
| 550 | <6.5 | >75 | >5 | <75 |
| 551 | >6.5 | >75 | >5 | <75 |
| 552 | <6.5 | <75 | <5 | <75 |
| 553 | <6.5 | >75 | >5 | <75 |
| 554 | >6.5 | >75 | >5 | >75 |
| 555 | >6.5 | <75 | >5 | <75 |
| 556 | >6.5 | >75 | >5 | <75 |
| 557 | <6.5 | >75 | >5 | <75 |
| 558 | >6.5 | >75 | >5 | >75 |
| 559 | >6.5 | >75 | >5 | >75 |
| 560 | >6.5 | >75 | >5 | >75 |
| 561 | >6.5 | >75 | >5 | <75 |
| 562 | >6.5 | >75 | >5 | >75 |
| 563 | >6.5 | >75 | >5 | <75 |
| 564 | >6.5 | >75 | >5 | >75 |
| 565 | >6.5 | >75 | >5 | >75 |
| 566 | <6.5 | <75 | <5 | <75 |
| 567 | <6.5 | >75 | <5 | <75 |
| 568 | >6.5 | >75 | >5 | >75 |
| 569 | <6.5 | >75 | <5 | <75 |
| 570 | <6.5 | <75 | <5 | <75 |
| 571 | >6.5 | >75 | >5 | <75 |
| 572 | <6.5 | <75 | <5 | >75 |
| 573 | <6.5 | <75 | <5 | <75 |
| 574 | <6.5 | >75 | <5 | >75 |
| 575 | >6.5 | >75 | >5 | >75 |
| 576 | >6.5 | >75 | >5 | <75 |
| 577 | <6.5 | <75 | >5 | <75 |
| 578 | <6.5 | >75 | <5 | <75 |
| 579 | >6.5 | >75 | >5 | >75 |
| 580 | >6.5 | >75 | >5 | >75 |
| 581 | <6.5 | >75 | <5 | <75 |
| 582 | <6.5 | >75 | <5 | <75 |
| 583 | <6.5 | <75 | <5 | <75 |
| 584 | >6.5 | >75 | >5 | >75 |
| 585 | >6.5 | >75 | >5 | >75 |
| 586 | >6.5 | >75 | >5 | >75 |
| 587 | <6.5 | <75 | <5 | <75 |
| 588 | <6.5 | <75 | <5 | <75 |
| 589 | <6.5 | <75 | <5 | >75 |
| 590 | <6.5 | <75 | <5 | <75 |
| 591 | >6.5 | <75 | >5 | >75 |
| 592 | <6.5 | <75 | <5 | >75 |
| 593 | >6.5 | >75 | >5 | >75 |
| 594 | >6.5 | >75 | >5 | >75 |
| 595 | >6.5 | >75 | >5 | >75 |
| 596 | >6.5 | <75 | >5 | >75 |
| 597 | >6.5 | >75 | >5 | >75 |
| 598 | >6.5 | >75 | >5 | >75 |
| 599 | >6.5 | <75 | >5 | >75 |
| 600 | >6.5 | >75 | >5 | >75 |
| 601 | <6.5 | >75 | >5 | <75 |
| 602 | <6.5 | <75 | <5 | >75 |
| 603 | >6.5 | >75 | >5 | >75 |
| 604 | >6.5 | >75 | >5 | >75 |
| 605 | >6.5 | >75 | >5 | >75 |
| 606 | >6.5 | >75 | >5 | >75 |
| 607 | >6.5 | >75 | >5 | >75 |
| 608 | >6.5 | >75 | >5 | >75 |
| 609 | >6.5 | <75 | <5 | <75 |
| 610 | <6.5 | <75 | <5 | >75 |
| 611 | >6.5 | <75 | >5 | <75 |
| 612 | >6.5 | <75 | >5 | >75 |
| 613 | <6.5 | <75 | <5 | <75 |
| 614 | <6.5 | >75 | <5 | <75 |
| 615 | >6.5 | >75 | >5 | >75 |
| 616 | >6.5 | >75 | >5 | >75 |
| 617 | <6.5 | <75 | <5 | >75 |
| 618 | <6.5 | <75 | >5 | <75 |
| 619 | >6.5 | >75 | >5 | >75 |
| 620 | >6.5 | <75 | >5 | >75 |
| 621 | <6.5 | >75 | <5 | >75 |
| 622 | >6.5 | >75 | <5 | <75 |
| 623 | <6.5 | >75 | <5 | >75 |
| 624 | <6.5 | <75 | <5 | >75 |
| 625 | <6.5 | <75 | <5 | <75 |
| 626 | <6.5 | <75 | <5 | <75 |
| 627 | <6.5 | >75 | <5 | >75 |
| 628 | >6.5 | >75 | >5 | <75 |
| 629 | <6.5 | >75 | <5 | <75 |
| 630 | <6.5 | <75 | <5 | <75 |
| 633 | <6.5 | <75 | <5 | <75 |
| 634 | <6.5 | <75 | <5 | <75 |
| 635 | >6.5 | <75 | >5 | <75 |
| 636 | >6.5 | >75 | >5 | >75 |
| 637 | >6.5 | >75 | >5 | >75 |
| 638 | >6.5 | <75 | <5 | <75 |
| 639 | <6.5 | <75 | <5 | >75 |
| 640 | <6.5 | >75 | <5 | <75 |
| 641 | <6.5 | <75 | <5 | <75 |
| 642 | <6.5 | <75 | <5 | <75 |
| 643 | <6.5 | >75 | <5 | <75 |
| 644 | <6.5 | >75 | <5 | >75 |
| 645 | <6.5 | >75 | <5 | <75 |
| 646 | <6.5 | <75 | <5 | >75 |
| 647 | >6.5 | >75 | >5 | >75 |

### Example 3: Compounds are Expected to be Effective to Protect Animals from the Induced Seizures in 6 Hz 44 mA Psychomotor Seizure Model, Mouse

Compounds are tested for efficacy in 6 Hz 44 mA Psychomotor Seizure Model, Mouse. In the 6 Hz model, the 6 Hz 44 mA model assesses the ability of a compound to prevent seizures induced by 6 Hz corneal stimulation at the 44 mA current intensity. These seizures are believed to model partial seizures observed in humans. The 6 Hz test employs an identical approach to that described for the MES test. Mice are challenged with a 44 mA current (2 times the CC97) for 3 sec delivered through corneal electrodes to elicit a psychomotor seizure (1). Typically, the seizure is characterized by an initial momentary stun followed immediately by jaw clonus, forelimb clonus, twitching of the vibrissae, and Straub tail lasting for at least 1 second. Animals not displaying this behavior are considered “protected”.

Initial qualitative screen for anticonvulsant activity in the 6 Hz seizure model is performed with N=4 male CF-1 mice/dose/time point. The default doses and time-points are 0.1, 1, 10 mg/kg s.c. at 0.5 and 2 hour following administration. Doses and/or time-points are adjusted if supported by other test data. Quantification of the ED50 is conducted at the time of peak effect (TPE). To determine the TPE, mice are treated with the investigational compound at 0.25, 0.5, 1.0, 2.0 and 4.0 hours, or based on time-points from prior studies. Groups of N=8 mice are tested with various doses of the investigational compound until at least two points can be clearly established between the limits of 100% protection or toxicity and 0% protection (i.e. at least 4 test doses). The data for each condition is presented as N/F, where N=number of animals protected and F=number of animals tested. The ED50, 95% confidence interval, the slope of the regression line, and the S.E.M. of the slope are calculated by Probit analysis.

### Example 4: Compounds are Expected to be Effective to Protect Animals from the Induced Seizures in Subcutaneous Metrazol Seizure Model and Intravenous Metrazol Seizure Threshold Model, Mouse

Compounds are tested for efficacy in Subcutaneous Metrazol Seizure Model and Intravenous Metrazol Seizure Threshold Model, Mouse.

Subcutaneous Metrazol Seizure Threshold Test (s.c. MET)

The s.c. MET test detects the ability of a test compound to raise the chemoconvulsant-induced seizure threshold of an animal and thus protect it from exhibiting a clonic, forebrain seizure. A dose of Metrazol 85 mg/kg for mice and 56.4 mg/kg for rats injected into a loose fold of skin in the midline of the neck. The animals are placed in isolation cages to minimize stress and observed for the next 30 min for the presence or absence of a seizure. An episode of clonic spasms, approximately 3 to 5 seconds, of the fore and/or hind limbs, jaws, or vibrissae is taken as the endpoint. Animals not displaying fore and/or hind limb clonus, jaw chomping, or vibrissae twitching are considered protected.

Initial qualitative screen for anticonvulsant activity in the s.c. MET test is performed with N=4 male CF-1 mice/dose/time point. The default doses and time-points are 1, 2.5, 5, 7.5, 10 mg/kg s.c. at 0.5 and 2 hr following administration. Doses and/or time-points are adjusted if supported by other test data. Quantification of the ED50 is conducted at the time of peak effect (TPE). To determine the TPE, mice are treated with the investigational compound at 0.25, 0.5, 1.0, 2.0 and 4.0 hrs, or based on time-points from prior studies. Groups of N=8 mice are tested with various doses of the investigational compound until at least two points can be clearly established between the limits of 100% protection or toxicity and 0% protection (i.e. at least 4 test doses). The data for each condition is presented as N/F, where N=number of animals protected and F=number of animals tested. The ED50, 95% confidence interval, the slope of the regression line, and the S.E.M. of the slope are calculated by Probit analysis.

Intravenous Metrazol Seizure Threshold Test (i.v. MET)

This test is not routinely performed but is useful for evaluating the effects of an investigational compound on MET. That is, whether a compound increases or decreases the threshold at which a seizure can be induced by MET. The timed i.v. infusion of MET to mice is used as a chemoconvulsant test to differentiate those compounds that lower seizure threshold and, as such, may be proconvulsant, from those compounds that elevate seizure threshold, and are thus anticonvulsant. Male CF1 mice (n=10 per dose level) are injected 2 minutes apart with either the vehicle or one of two doses (ED50 and TD50; i.p.) of the test compound previously determined to be effective in screening seizure tests and motor impairment assessment. During the drug dosing, animals maintain the same order of dosing until all mice have been injected according to the method described. At the previously determined TPE, 0.5% MET solution is then infused at a constant rate of 0.34 ml/min through a length of No. 20 PE tube cannulating a lateral tail vein of a mouse. At the start of the MET infusion, a hemostat clamped to the guide tubing to prevent backflow is removed, the infusion started, and two stopwatches started. The time in seconds from the start of the infusion to the appearance of the “first twitch” and the onset of sustained clonus is recorded or 90 seconds if the time has passed with no seizure. The mean and S.E.M for each of the 3 groups (vehicle, ED50 and TD50), and the significant difference between the test groups and the control is calculated. An increase in mg/kg to first twitch or to clonus indicates the test substance increases seizure threshold; whereas a decrease indicates that the test substance decreases seizure threshold, or may be proconvulsant.

### Example 5: Compounds are Expected to be Effective to Protect Animals from the Induced Seizures in Corneal Kindled Seizure Model, Mouse

Compounds are tested for efficacy in Corneal Kindled Seizure Model (CKM) Mouse. The pharmacological profile of the corneal kindled mouse is consistent with human partial epilepsy and effectively identifies the anticonvulsant potential of useful compounds for this condition, such as levetiracetam. Male C57BL/6 mice are kindled electrically with 3 sec stimulation, 3 mA, 60 Hz, and corneal electrodes to a criterion of 5 consecutive Stage 5 seizures (facial clonus and head nodding progressing to forelimb clonus, and finally rearing and falling accompanied by a generalized clonic seizure). After receiving twice daily corneal stimulations, mice typically reach the first Stage 5 seizure between approximately 10-14 days. Twice daily stimulations continue for each mouse until that mouse has achieved the criterion of 5 consecutive stage 5 seizures, whereby it is considered “fully kindled”. Fully kindled mice are then stimulated every-other day until all other mice within the group reach the criterion of 5 consecutive Stage 5 seizures. Any mouse not achieving the fully kindled state is not included in any evaluation of investigational compounds.

Testing of investigational compounds commences at least 5-7 days after the last stimulation. Mice are stimulated on the day prior to any drug evaluation to ensure that all mice to be used in the drug study will present with a Stage 5 seizure. For identification studies, the test compound is administered to a group of 8 fully kindled mice per group at 3 and 5 mg/kg s.c. and tested at time-points 0.5 and 1 hr following administration¬Based on the experimental results, the compound is then retested to determine the ED50 value. After testing, the corneal kindled animals are returned to their home cage. Unlike the acute seizure tests conducted by the ETSP, each corneal kindled mouse is allowed at least 3-4 days between tests to “washout” any investigational compound after testing.

### Example 6: Compound 469 can Inhibit Seizures

Compound 469 was tested in the method described in Example 5. The data for Compound 469 is illustrated in Tables 1-4. Compound 469 was found to have an ED₅₀ of 6.2 mg/kg at 2 hour time point in Corneal Kindled Seizure Model.

TABLE 1
Experiment 1: Seizure scores of 3 and 5 mg/kg of compound 469at 0.5
hour and 1.0 hour time points in Corneal Kindled Seizure Model.

| Dose | Time | | Individual Seizure | Average Seizure |
|---|---|---|---|---|
| (mg/kg) | (hour) | N/F | Scores | Scores |
| 3 | 0.5 | 0/8 | 5, 3, 5, 5, 5, 5, 3, 5 | 4.5 |
| 5 | 0.5 | 0/8 | 3, 5, 5, 5, 3, 5, 3, 3 | 4 |
| 3 | 1 | 1/8 | 4, 3, 0, 5, 5, 3, 3, 4 | 3.38 |
| 5 | 1 | 1/8 | 5, 3, 2, 3, 5, 3, 3, 3 | 3.38 |

N: number of animals protected or toxic.
F: total number of animals tested.

TABLE 2
Experiment 2: Seizure scores of various doses of Compound
469 at 2 hrs in Corneal Kindled Seizure Model.

| Dose | Time | | Individual Seizure | Average Seizure |
|---|---|---|---|---|
| (mg/kg) | (hrs) | N/F | Scores | Scores |
| 1 | 2 | 1/8 | 3, 3, 4, 1, 3, 4, 4, 4 | 3.25 |
| 2.5 | 2 | 2/8 | 5, 3, 3, 2, 3, 5, 2, 4 | 3.38 |
| 5 | 2 | 3/8 | 4, 5, 5, 0, 3, 0, 3, 0 | 2.5 |
| 7.5 | 2 | 3/8 | 5, 3, 0, 0, 0, 3, 3, 3 | 2.13 |
| 10 | 2 | 7/8 | 0, 0, 3, 2, 0, 0, 0, 0 | 0.63 |

N: number of animals protected or toxic.
F: total number of animals tested.

TABLE 3
ED50 of Compound 469 at 2 hrs time point
n Corneal Kindled Seizure Model.

| | Time | ED50 | 95% Confidence | | |
|---|---|---|---|---|---|
| Test | (hrs) | (mg/kg) | Interval | Slope | Std Err |
| CKM | 2 | 6.2 | 3.21-20.87 | 1.76 | 0.676 |

TABLE 4
Seizure scores of 10 mg/kg of Compound 469 at 1 hour and
2.0 hour time points in Corneal Kindled Seizure Model.

| Dose | Time | | Individual Seizure | Average Seizure |
|---|---|---|---|---|
| (mg/kg) | (hrs) | N/F | Scores | Scores |
| 10 | 1 | 2/8 | 3, 3, 1, 3, 3, 3, 0, 3 | 2.38 |
| 10 | 2 | 7/8 | 0, 0, 3, 2, 0, 0, 0, 0 | 0.63 |

These data demonstrate that the compounds provided herein, such as Compound 469 can reduce seizures, and, therefore, can be used to treat seizure disorders, such as those provided for herein.

### Example 7: Compounds are Expected to be Effective to Protect Animals from the Induced Seizures in Lamotrigine (LTG) Resistant Amygdala Kindled Seizure Model, Rat

Compounds are tested for efficacy in Lamotrigine (LTG) Resistant Amygdala Kindled Seizure Model, Rat. The LTG-resistant amygdala kindled rat model is useful for not only identifying compounds effective against secondarily generalized partial seizures, but also allows for the differentiation of compounds that may be effective in therapy-resistant patients. Daily administration of lamotrigine (LTG; 5 mg/kg) during the kindling acquisition phase does not prevent the development of kindling in the test animals but leads to a LTG-resistant state in the fully kindled rat. Other sodium channel blockers, such as phenytoin and carbamazepine, also do not block kindling acquisition despite being highly effective against fully kindled seizures in drug-naïve rats. Conversely, valproate can both prevent kindling development and block the expression of fully kindled seizures. The addition of the traditional ASDs, carbamazepine or lamotrigine, during the development of kindled seizures in this model will ultimately impair the effectiveness of lamotrigine against a fully expressed kindled seizure. These findings suggest that the presence of lamotrigine during the epileptogenic process leads to a subsequent resistance to other Na+ channel blockers, thereby making this a useful model of pharmacoresistance. This model may serve as a means to identify compounds, which may be effective against therapy-resistant seizures. Anesthetized male Sprague-Dawley rats (250-300 g) are surgically implanted with an electrode into the left amygdala. Animals are then allowed to recover for one week before commencing kindling. The kindling procedure consists of delivering a 200 pAmp stimulus (suprathreshold) daily until all animals in both treatment groups display consistent Stage 4 or 5 seizures. One week after all animals are kindled, the animals receive a challenge dose of LTG (30 mg/kg, i.p.) before being stimulated to confirm the LTG sensitivity of the vehicle-treated control animals, as well as the LTG-resistance of the LTG-treated group. The animals are then allowed a washout of 3 days. On day 3 of the washout, the animals are pre-stimulated to ensure recovery of the fully kindled seizure. On day 4, kindled rats are challenged with a dose of an investigational agent (the dose that produced minimal motor impairment) and then challenged with the kindling stimulus at the predetermined TPE of the investigational drug. When a drug treatment is observed to significantly lower seizure score and decrease afterdischarge, a dose-response study can be conducted. For this study, the ability of a candidate substance to reduce afterdischarge duration (ADD) and behavioral seizure scores (BSS) is quantitated by varying the dose between 0 and 100% effect. Unlike other acute seizure tests conducted by the ETSP, each kindled rat is allowed at least 3-4 days between tests to “washout” any investigational compound after testing. The average seizure scores f S.E.M. and afterdischarge duration (ADD) are noted, as are the number of animals protected from seizure (defined as a Racine score <3) over the number of animals tested.

The following procedure is used for surgical implantation of Electrodes. Male Spraque-Dawey rats are anesthetized using 1-3% isoflurane or a Ketamine/Xylazine cocktail. Ketamine is provided as 100 mg/ml solution, and dose 50 mg/kg given i.p. Xylazine is provided as 100 mg/ml solution, and dose 20 mg/kg given i.p. Mix the two solutions together in one syringe and administer i.p as one injection. Rat is monitored through surgery. If the rat is responsive to a tail pinch administer half the dose of Ketamine/xylazine or increase the isoflurane percentage. Administer buprenorphine at 0.02 mg/kg s.c. for pain management. An electric razor is used to shave the surface of the head. The rat is placed on an external heat source on the stereotaxic apparatus such that the height of the incisor bar and the positioning of the ear bars ensure consistency with the reference atlas. The entire scalp is scrubbed with betadine (3×) and wiped with alcohol. Ophthalmic ointment is applied to each eye. All instruments are sterilized via autoclave before beginning surgery. During surgery instruments are placed on a sterile drape. For subsequent surgeries on the same day, instruments can be placed into the hot bead sterilizer between surgeries. Stainless steel screws and electrodes are sterilized in 70% alcohol and place on a sterile drape. A sterile scalpel blade is used to make a midline scalp incision beginning from a point even with the eyes and extending back to and imaginary line connecting the ear. The fascia should be gently split and pulled away by from the scalp. The incision can be held open using retractors/forceps. Screw holes are drilled without penetrating the dura using a Dermal drill and four anchor screws are attached to the skull. The bipolar stimulating electrode is implanted through a fourth hole drilled in the left amygdala (anterio-posterior, AP, +5.7 mm, medio-lateral, ML, +4.5 mm, dorso-ventral, DV, +2.0 mm from intra-aural zero). The electrode assembly is anchored to the skull via stainless steel screws with dental acrylic cement. After the incision is closed with sutures, antibiotic ointment is applied around the incision site. The rat is given a dose of penicillin 60,000 units s.c. and rimidyl injectable 0.03 mg/kg. Rat is left on the heat source until ambulatory.

### Example 8: Compounds are Expected to be Effective to Protect Animals from the Induced Seizures in Pilot Study of Status Epilepticus-Induced Spontaneous Recurrent Seizures Measured with Video-EEG Monitoring, Rat

Compounds are tested for efficacy in Pilot study of Status Epilepticus-Induced Spontaneous Recurrent Seizures Measured with Video-EEG Monitoring, Rat

### Induction of Chronic Epilepsy:

48 Sprague-Dawley rats are induced with Status Epilepticus using a repeated low-dose Kainic Acid (KA) paradigm. Rats are injected with 7.5 mg/kg of KA intraperitoneal (i.p.) at 0 h, 1 h, and every subsequent half-hour (up to 4 h) or until the animal displays two Racine Stage 5 seizures. The dose may be reduced to ½ or ⅓ as necessary, as the animal exhibits lower-stage seizures. At the end of the injection period, rats are given 3 ml of Ringers solution to prevent dehydration. It is expected that approximately 36 rats will survive this treatment.

Implantation of Telemeter after 10 weeks, rats are implanted with a Millar wireless telemeter. The telemeter is implanted into the peritoneal space. The EEG cable is routed from the stomach to the head underneath the skin. Three holes are drilled in the skull, and three fixation screws are placed. Two additional holes are drilled, and an EEG wire is placed in each. The wires are secured via super glue. The skull is sutured shut, and then the excess wire is coiled in the peritoneal space and the stomach is sutured shut. Rats are placed in the EEG suites, and an initial seizure rate is acquired over a week. The 24 rats with the highest seizure rates will be selected. Rats with seizure burden scores (seizure burden is calculated as the sum total of all seizure scores divided by the number of days tested) of lower than 10 per day are removed from future studies.

Stage 1 Chronic Monitoring: In this paradigm, rats are injected (i.p.), subcutaneous (s.c.) or oral (p.o.) with vehicle or drug, twice or three times a day, based on the known pharmacokinetic profile of the drug being tested (if available) or the time-to-peak effect (TPE) of the drug in previously evaluated seizure models. (It is anticipated that the lamotrigine-resistant amygdala kindling model will be used as a primary guide for determination of a treatment strategy in Stage 1 chronic monitoring studies). In the first week (week 1), a baseline seizure rate is determined. During the following week (week 2), injections are performed over 5 days, Monday-Friday. Rats are split into groups of 8-12 (vehicle- and drug-treated groups). After treatment is completed in week 2, rats will be monitored during week 3 (washout period). This process (week 1-3) will be repeated for up to 5 separate testing runs.

Possible design variations include (1) Cross-over paradigm: one group receiving drug for the first 5 days (during week 2), and vehicle for the subsequent five days (week 3). Similarly, the second group receives vehicle first, followed by drug. Rats will then enter into a one-week washout period (week 4). (2) Group sizes may vary and will be updated once an initial power analysis is completed to verify numbers required to achieve statistical significance. For example, an initial group of 24 rats enrolled in Stage 1 chronic monitoring can be split into two (N=12/group) or three (N=8/group) treatment groups.

### Data Review and Analysis

EEG data is reviewed daily, in a blinded fashion. Data channel order is randomly scrambled and unlabeled. A list of potential detected events are automatically generated overnight by an automated seizure detection algorithm. The reviewer goes through these detected listings in a sequential order, and scores any positive detected events. Data is accumulated at the end of the paradigm, and analyzed via a MATLAB GUI. Factors analyzed include seizure burden, frequency, and distribution of Racine scores.

### Example 9: Compounds are Expected to be Effective to Protect Animals from the Induced Seizures in Oral Dosing Study of Status Epilepticus-Induced Spontaneous Recurrent Seizures Measured with Video-EEG Monitoring, Rat

Compounds are tested for efficacy in Oral Dosing Study of Status Epilepticus-Induced Spontaneous Recurrent Seizures Measured with Video-EEG Monitoring, Rat. This paradigm utilizes the automated feeder system to deliver drug-in-food on a fixed schedule. The induction of chronic epilepsy and implantation of the telemeter in the rats is performed the same as described for the pilot study. Animals are split into two groups and given 45 g/kg per day of either drug or control food for two weeks. The amount of drug in each gram of food is fixed to make the appropriate dose. The number of “meals,” i.e. automated pellet distributions, will be based on the known pharmacokinetic profile of the drug being tested (if available) or the time-to-peak effect (TPE) of the drug in previously evaluated seizure models. In the first week (week 1), a baseline seizure rate is determined. During the following two weeks (week 2-3), feedings are given based on the rat's group. After treatment is completed in week 3, rats will be monitored during week 4 (washout period). This process (week 1-4) will be repeated for up to 4 separate testing runs.

### Data Review and Analysis

EEG data is reviewed daily, in a blinded fashion. Data channel order is randomly scrambled and unlabeled. A list of potential detected events are automatically generated overnight by an automated seizure detection algorithm. The reviewer goes through these detected listings in a sequential order, and scores any positive detected events. Data is accumulated at the end of the paradigm, and analyzed via a MATLAB GUI. Factors analyzed include seizure burden, frequency, and distribution of Racine scores.

### Example 10: Compounds are Expected to be Effective to Protect Animals from the Induced Seizures in PTZ (Pentylenetetrazol) Test

Compounds are tested for efficacy in PTZ (Pentylenetetrazol) Test. In the experiment, administered intraperitoneally (i.p.) or orally to test animals (Mouse; ICR, and Rats; SD); Experimental animal, male SD rats, were purchased from OrientBio or Nara biotech, Korea, and housed 4-5 mice per a cage for 4-5 days. The range of mice body weight was used between 19 and 26 grams and range of rats body weight was used between 100 and 130 grams. After Peak time (0.5, 1, 2 and 4 hr) from the administration, from the administration, PTZ (Pentylenetetrazol) was administered subcutaneously in the concentration capable of inducing 97% intermittent convulsions (mice & rats: 90˜110 mg/kg bw, 2 μl/g). If clonic seizure was not observed for at least 3 seconds in the PTZ administered animal, it can be considered that the test compound has nonconvulsive seizure activity. The median effective dose (ED50) is determined using 6 animals per a concentration (total three different concentrations), and calculated by Litchfield and Wicoxon log-probit method which is a dose-response relationship.

### Example 11: Compounds are Expected to be Effective to Protect Animals from the Induced Seizures in Minimal Clonk Seizure (6 Hz) Test

Compounds are tested for efficacy in Minimal Clonic Seizure (6 Hz) Test. Some clinically useful AEDs are ineffective in the standard MES and scPTZ tests but still have anticonvulsant activities in vivo. In order to identify potential AEDs with this profile, compounds may be tested in the minimal clonic seizure (6 Hz or ‘psychomotor’) test (Barton et al., 2001). Like the maximal electroshock (EMS) test, the minimal clonic seizure (6 Hz) test is used to assess a compound's efficacy against electrically induced seizures but used a lower frequency (6 Hz) and longer duration of stimulation (3 s).

Test compound was pre-administrated to mice via i.p. injection. At varying times, individual mice (four per time point) are challenged with sufficient current delivered through corneal electrodes to elicit a psychomotor seizure in 97% of animals (32 mA or 44 mA for 3 s) (Toman et al., 1952). Untreated mice will display seizures characterized by a minimal clonic phase followed by stereotyped, automatistic behaviors described originally as being similar to the aura of human patients with partial seizures. Animals not displaying this behavior are considered protected. The test may be evaluated quantitatively by measuring the response at varying doses at a determined time of peak effect (TPE). (Reference; Barton M. E., Klein B. D., Wolf H. H. and White H. S. (2001) Pharmacological characterization of the 6 Hz psychomotor seizure model of partial epilepsy, Epilepsy Res. 47: 217-227./Toman J. E., Everett G. M. and Richards R. K. (1952), The search for new drugs against epilepsy. Tex. Rep. Biol. Med. 10: 96-104.).

### Example 12: Compounds are Expected to be Effective to Protect Animals from the Induced Seizures in Lithium-Pilocarpine Induced Status Epilepticus Model

Compounds are tested for efficacy in Lithium-Pilocarpine Induced Status Epilepticus Model

### Prevention Study

Male Sprague-Dawley rats (purchased from Orient Bio Inc. Korea) of body weight 200-230 g were used for these studies and housed 4-5 rats per a cage for 4-5 days. On the day prior to status epilepsy (SE), rats received 127 mg/kg lithium chloride (Sigma, St. Louis, Mo., U.S.A.) intraperitoneally (i.p.). Approximately 18-20 h following this treatment, the rats were given 43 mg/kg pilocarpine (Sigma) intraperitoneally. An i.p. injection of 2 mg/kg methyl-scopolamine (Sigma) was administered 30 min prior to pilocarpine to block the effects of the muscarinic agonist on peripheral cholinergic receptors. The test drug was administered intraperitoneally (i.p.) in a volume of 2 ul/g body weight. Pharmacological effects of all the test materials were evaluated to compare the test groups (n=6) with a control group (n=6). Control group was administrated vehicle, only. The peak time was determined by administration test material's random dose for 0.5, 1, 2, 4 hour. The time that the most protect was defined peak time and ED50 was determined by other dose administration at peak time. The animals were then transferred to observation cages and observed continuously for 90 min. The seizure activity was elicited in approximately 95% of control group. Protection was defined as a complete absence of seizure grade 4^{˜}5 based on Racine scale (Racine, 1972) over the 90-min observation period. The effective dose of compound necessary to protect against seizures to 50% of controls (i.e. ED50) was determined by log probit analysis using SPSS software program (SPSS Inc.).

### Intervention Study

Male Sprague-Dawley rats (purchased from Orient Bio Inc. Korea) of body weight 200-230 g were used for these studies and housed 4-5 rats per a cage for 4-5 days. On the day prior to SE, rats received 127 mg/kg lithium chloride (Sigma, St. Louis, Mo., U.S.A.) intraperitoneally (i.p.). Approximately 18-20 h following this treatment, the rats were given 43 mg/kg pilocarpine (Sigma) intraperitoneally. An i.p. injection of 2 mg/kg methyl-scopolamine (Sigma) was administered 30 min prior to pilocarpine to block the effects of the muscarinic agonist on peripheral cholinergic receptors. The effects of the test compounds dissolved in 30% Poly Ethylene Glycol 400 (Acros Organics, Geel, Belgium) or 20% Tween80 were studied at various times or 30 min after the occurrence of the first motor seizure or SE onset. The drug was administered intraperitoneally in a volume of 2 ul/g body weight. Pharmacological effects was evaluated to compare the test groups with a control group (n=8). Control group was administrated vehicle, only. (Reference; Racine R. J. (1972). Modification of seizure activity by electrical stimulation: II Motor seizure. Electroenceph. Clin. Neurophysiol. 32: 281-294.)

### Example 13: Compounds Are Expected to be Potential Pharmacological Therapies for Benzodiazepine-Resistant Status Epilepticus

Compounds are tested for efficacy in the study for Potential Pharmacological Therapies for Benzodiazepine-Resistant Status Epilepticus.

The lithium-pilocarpine model is used to study the effects of test compounds on the electrographic properties of benzodiazepine-resistant SE. Adult rats are implanted for electroencephalogram (EEG) recordings, and then pretreated with lithium chloride (127 mg/kg, 24 h) and scopolamine bromide (1 mg/kg; 30 min) prior to the administration of pilocarpine (50 mg/kg). Either thirty or sixty minutes after the development of the first motor seizure, the animals receive diazepam (10 mg/kg). Ten minutes after diazepam, the experimental group is given the test compound and the control group given vehicle. Typically, 8 animals comprise a “Trial” where 2 animals are controls (i.e., vehicle only) and 6 animals receive the test compound, although this varies depending on the number animals that actually experience SE. In order to have an adequate number of replications, two (or even more) Trials are conducted. When needed, additional control animals are derived from other temporally adjacent Trials using the same protocol.

### Example 14: Compounds Selective for S1P1 Receptor

### In Vitro Selectivity Assays

Stable clonal Chinese hamster ovary K1 (CHO-K1) cells co-expressing EA-β-arrestin2 and the human sphingosine-1-phosphate receptor 2 (NM_004230.3, S1P2), human sphingosine-1-phosphate receptor 3 (NM 005226, S1P₃) and sphingosine-1-phosphate receptor 5 (NM_001166215.1, S1P₅) with a C-terminal Prolink™ tag were purchased from DiscoverX corporation (S1P2: Cat #93-0256C2, S1P3: Cat #93-0217C2, SiP5: Cat #93-0583C2).

Cell Culturing and Assay Plating Cell lines were cultured in AssayComplete™ Media 6 (DiscoverX Corporation, Cat #: 920018GF2) at 37° C. and 5% CO₂ in a humidified CO₂ and temperature-controlled incubator. To begin assay plating, cells were washed with Dulbecco's phosphate buffered saline (CellGro, Cat #: 21-031-CV) and lifted from the culturing flask by incubation (37° C., 5 min) with CellStripper (Cellgro, Cat #: 25-056-CI). Lifted cells were resuspended to 250,000 cells per milliliter in either AssayComplete™ Cell Plating 11 Reagent (S1P5 cell line) (DiscoverX Corporation, Cat #: 93-0563R11B) or AssayComplete™ Cell Plating 2 Reagent (S1P2 and S1P3 cell line) (DiscoverX Corporation, Cat #: 93-0563R2B) and plated at 5000 cells per well (SIP3 cell line) or 7500 cells per well (S1P2 and S1P5 cell line) in white-opaque 384 well plates (Greiner Bio-One Item #: 20-784080). Plated cells were incubated overnight at 37° C. and 5% CO₂ in a humidified CO₂ and temperature-controlled incubator.
Detecting Inhibition of cAMP Production

S1P3 and S1P5 Agonist-promoted G-protein responses were determined by measuring changes in intracellular cAMP using the HTRF® cAMP HiRange kit (CisBio, Cat #: 62AM6PEJ) based on time-resolved fluorescence resonance energy transfer (TR-FRET) technology. AssayComplete™ Cell Plating 11 Reagent was removed and replaced with Ham's F-12 (CellGro, Cat #: 10-080-CM) containing isobutyl-methyl-xanthine (IBMX; 500 μM; Tocris Bioscience, Cat #: 2845), and NKH-477 (1.5 μM; Tocris Bioscience, Cat #: 1603) along with test or control compounds at the desired concentrations. Following a 30-minute room temperature incubation the components of the cAMP HiRange kit were added as per the manufacturer's instructions. After an hour incubation at room temperature, plates were analyzed by a BMG PheraStar microplate reader. Responses were measured as the ratio of signal over background, fluorescence emission at 665 nm to fluorescence emission at 620 nm.

### Detecting Inositol Monophosphate Production

S1P2 Agonist-promoted G-protein responses were determined by measuring changes in intracellular inositol monophosphate using the IP-one TB kit (CisBio, Cat #: 62IPAPEJ) based on time-resolved fluorescence resonance energy transfer (TR-FRET) technology. AssayComplete™ Cell Plating 2 Reagent was removed and replaced with 1× IP-one stimulation buffer (as per manufacturer's instructions) along with test or control compounds at the desired concentrations.

Following a 60-minute incubation at 37° C. and 5% CO2 in a humidified CO2 and temperature-controlled incubator, the components of the IP-one TB kit were added as per the manufacturer's instructions. After an hour incubation at room temperature, plates were analyzed by a BMG PheraStar microplate reader. Responses were measured as the ratio of signal over background, fluorescence emission at 665 nm to fluorescence emission at 620 nm.

### Activity Table

The compounds were able to modulate the activities (inhibition of cAMP production or accumulation of inositol monophosphate) of the sphingosine-1-phosphate 2, sphingosine-1-phosphate 3, sphingosine-1-phosphate 5 receptor as indicated herein. The tables below include the efficacy of the compound normalized to the maximal efficacy of a reference compound, referred to as “SPAN”. These values are normalized to fimgolimod, a known agonist of the sphingosine-1-phosphate 3 and 5 receptor or CYM5520, a known agonist of the sphingosine-1-phosphate 2 receptor. The tables also include potency values (pEC50) for modulating discrete receptor-mediated activities (inhibition of cAMP production or inositol monophosphate accumulation). This value represents the estimated concentration to promote half of the maximal efficacy (or SPAN) observed for each compound. Exemplary compounds that were found to be selective are shown below.

| | hS1P2 | hS1P2 | hS1P3 | hS1P3 | hS1P5 | hS1P5 |
|---|---|---|---|---|---|---|
| Compound | IP-one | IP-one | cAMP | cAMP | cAMP | cAMP |
| number | pEC₅₀ | SPAN | pEC₅₀ | SPAN | pEC₅₀ | SPAN |
| 22 | >5.0 | <50 | <5.0 | <50 | >5.0 | <50 |
| 26 | <5.0 | <50 | <5.0 | <50 | <5.0 | <50 |
| 32 | <5.0 | <50 | <5.0 | <50 | <5.0 | <50 |
| 99 | <5.0 | <50 | <5.0 | <50 | <5.0 | >50 |
| 103 | <5.0 | <50 | <5.0 | <50 | <5.0 | <50 |
| 141 | <5.0 | <50 | >5.0 | >50 | >5.0 | >50 |
| 147 | <5.0 | <50 | <5.0 | <50 | >5.0 | <50 |
| 149 | <5.0 | <50 | >5.0 | >50 | >5.0 | >50 |
| 152 | <5.0 | <50 | <5.0 | <50 | <5.0 | <50 |
| 177 | <5.0 | <50 | >5.0 | >50 | >5.0 | >50 |
| 185 | <5.0 | <50 | <5.0 | <50 | <5.0 | >50 |
| 212 | <5.0 | <50 | <5.0 | <50 | <5.0 | <50 |
| 219 | >5.0 | <50 | <5.0 | <50 | <5.0 | <50 |
| 221 | <5.0 | <50 | >5.0 | <50 | >5.0 | <50 |
| 252 | >5.0 | <50 | >5.0 | <50 | >5.0 | >50 |
| 259 | <5.0 | <50 | <5.0 | <50 | <5.0 | <50 |
| 262 | <5.0 | >50 | <5.0 | <50 | <5.0 | <50 |
| 263 | <5.0 | <50 | <5.0 | <50 | <5.0 | <50 |
| 267 | <5.0 | <50 | <5.0 | <50 | <5.0 | <50 |
| 277 | <5.0 | >50 | <5.0 | <50 | <5.0 | <50 |
| 287 | <5.0 | <50 | <5.0 | <50 | <5.0 | <50 |
| 293 | <5.0 | <50 | <5.0 | <50 | <5.0 | >50 |
| 304 | <5.0 | <50 | >5.0 | <50 | <5.0 | <50 |
| 391 | >5.0 | <50 | >5.0 | >50 | >5.0 | <50 |
| 398 | <5.0 | <50 | <5.0 | <50 | <5.0 | <50 |
| 406 | <5.0 | <50 | <5.0 | <50 | <5.0 | <50 |
| 417 | >5.0 | <50 | <5.0 | <50 | <5.0 | <50 |
| 422 | >5.0 | <50 | >5.0 | <50 | | |
| 423 | >5.0 | <50 | >5.0 | <50 | | |
| 445 | >5.0 | <50 | <5.0 | <50 | | |
| 455 | <5.0 | <50 | <5.0 | <50 | | |
| 496 | <5.0 | <50 | <5.0 | <50 | | |

Thus, the compounds were found to be sufficiently selective for S1P1.

### Example 15: Compounds do not Inhibit hERG Channel Activity

The standard automated Qpatch patch-clamp assay have been used and the selective hERG inhibitor E4031, serves as a positive control.

| Compound number | IC₅₀ (μM) |
|---|---|
| E4031 | 0.013 |
| 26 | >10 |
| 304 | >10 |
| 469 | >10 |
| 497 | >10 |
| 520 | >10 |
| 730 | >10 |
| 738 | >10 |
| 742 | >10 |
| 743 | >10 |
| 744 | >10 |

### Example 16: Compounds do not Cause Lymphopenia

Compounds were tested for changes in peripheral blood lymphocytes in C57bl/6 mice. In acute studies, animals (n=5/group) were dosed subcutaneously with test compound at a dose of 3 mg/kg. Animals were sacrificed at specific time points and 500 si of whole blood was collected in EDTA (K2) Eppendorf tubes. Blood was stored on ice and shipped immediately overnight delivery to Charles River Laboratories for analysis. CRL ran samples through their WBC/differential panel on an Advia 120 instrument. We received peripheral lymphocyte counts (10³ cells/μl) for each blood sample. Treatment group means were compared to a vehicle treatment group for statistical significance. In chronic studies, animals (n=6-8/group) were dosed subcutaneously for three to seven days with test compound at doses that were at least 5-fold higher than the efficacious dose in pharmacology/behavioral assays. On the last day, animals were sacrificed 45 minutes to 6 hrs after the final dose. Whole blood was collected and analyzed as described for acute studies. None of the compounds showed statistically significant decreases in peripheral blood lymphocytes in acute or chronic studies. Non-limiting exemplary data is provided below.

Lymphopenia - c57bl/6 mice

| Compound Number | Effect and Doses, mg/kg, sc |
|---|---|
| 8 | no change: 6 mg/kg, 7 days |
| 19 | no change: 6 mg/kg, 7 days |
| 22 | no change: 3 mg/kg, 7 days |
| 32 | no change: 6 mg/kg, 7 days |
| 55 | no change: 6 mg/kg, 7 days |
| 59 | no change: 6 mg/kg, 7 days |
| 96 | no change: 6 mg/kg, 7 days |
| 99 | no change: 6 mg/kg, 7 days |
| 103 | no change: 6 mg/kg, 7 days |
| 142 | no change: 6 mg/kg, 7 days |
| 147 | no change: 6 mg/kg, 7 days |
| 169 | no change: 6 mg/kg, 7 days |
| 234 | no change: 6 mg/kg, 7 days |
| 287 | no change: 6 mg/kg, 7 days |
| 293 | no change: 6 mg/kg, 7 days |
| 304 | no change: 6 mg/kg, 7 days |
| 355 | no change: 6 mg/kg, 7 days |
| 356 | no change: 6 mg/kg, 7 days |
| 384 | no change: 3 mg/kg; 45 min and 2 hr |
| 423 | no change: 6 mg/kg, 7 days |
| 455 | no change: 6 mg/kg, 7 days |
| 469 | no change: 3.7 mg/kg, 3 days |
| 520 | no change: 6 mg/kg, 7 days |
| 531 | no change: 6 mg/kg, 7 days |
| 730 | no change: 6 mg/kg, 7 days |
| 731 | −78% stat sig: 6 mg/kg, 7 days |
| 737 | −40% not stat sig: 6 mg/kg, 7 days |

1. A method of treating or preventing a seizure, or an epilepsy or an epilepsy-related syndrome in a subject in need thereof, the method comprising administering to the subject a compound having Formula I or Formula II, or a pharmaceutically acceptable salt thereof: 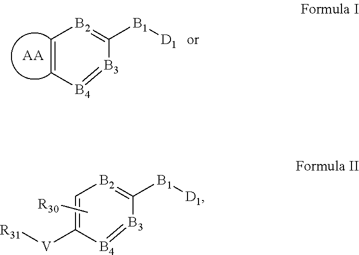
wherein:
AA is 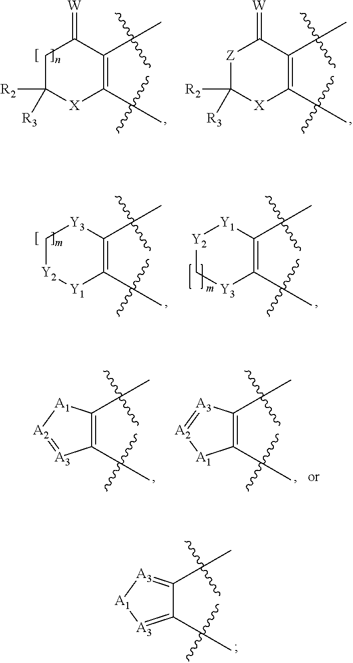
W is O, S, or NR₁;
X is O, S, or NR₄;
V is O, S, or NR₃₂;
Z is CHR₄₂ or NR₄₃;
n is 0, 1, 2, 3, or 4;
Y₁ and Y₂ are independently O, S, NR₅, C═O, C═S or C═NR₆;
Y₃ is O, S, CH₂, or NR₃₄;
m is 0, 1, 2, or 3;
A₁ is O, S, NR₇, C═O, or C═S;
A₂ and A₃ are independently CR₂₉ or N;
B₁ is an optionally substituted aryl or heteroaryl group, a carbocycle, or 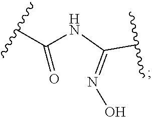 [SMILES: *C(=O)N/C(*)=N/O]
B₂, B₃, and B₄ are independently CR₃₈ or N;
D₁ is H, OH, NH₂, NO₂, cycle, optionally substituted aryl group, branched or unbranched alkyl alcohol, halo, branched or unbranched alkyl, amide, cyano, alkoxy, haloalkyl, aklylsulfonyl, nitrite, or alkylsulfanyl;
R₂ and R₃, are independently H, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₂ and R₃ are together optionally substituted cycloalkyl, or optionally substituted cycloheteroalkyl;
R₁, R₄, R₅, R₆, R₇, R₂₉, R₃₁, R₃₂, R₃₃, R₃₄, R₃₈, and R₄₃ are independently H, OH, NH₂, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl;
R₃₀ is independently H, CN, CF₃, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or optionally substituted haloalkyl; and
R₄₂ is independently Br, Cl, F, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl.

2. The method of claim 1, wherein D₁ and B₁ have a formula of: 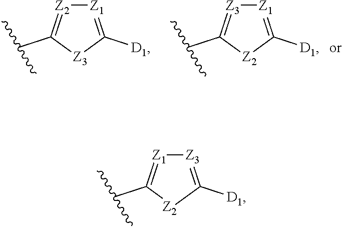
wherein:
Z₁ and Z₂ are independently N or CR₃₉;
Z₃ is O, S, or NR₂₇;
R₂₇ and R₃₉ are independently H, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; and
D₁ is H, OH, NH₂, NO₂, cycle, optionally substituted aryl group, branched or unbranched alkyl alcohol, halo, branched or unbranched alkyl, amide, cyano, alkoxy, haloalkyl, aklylsulfonyl, nitrite, or alkylsulfanyl.

3-4. (canceled)

5. The method of claim 2, wherein D₁ an B₁ together have a formula of

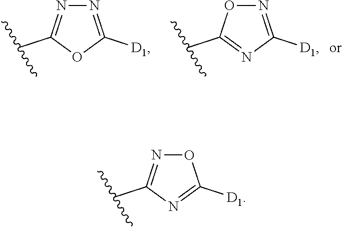

6-17. (canceled)

18. The method of claim 1, wherein AA is 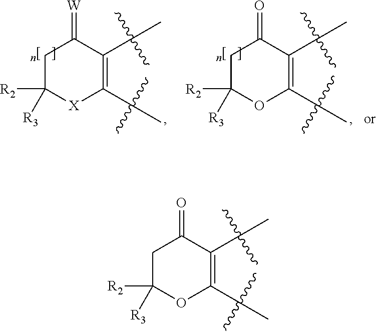
and wherein R₂ and R₃ are independently H, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl or R₂ and R₃ together form

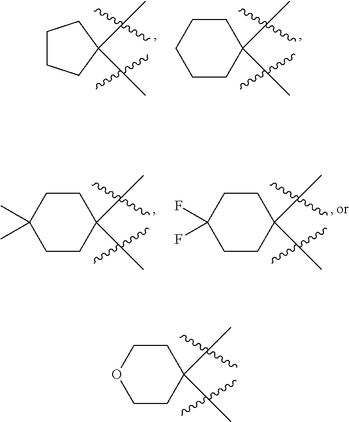

19-23. (canceled)

24. The method of claim 18, wherein D₁ is 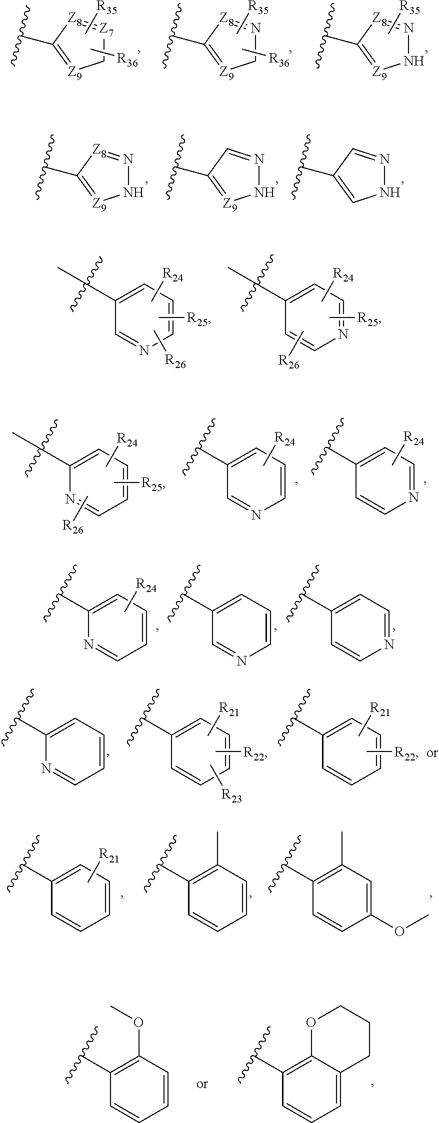
wherein the variables are as defined in claim 1.

25-55. (canceled)

56. The method of claim 1, wherein AA is 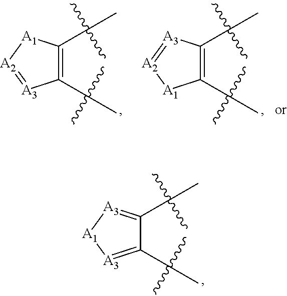
and wherein R₇ is

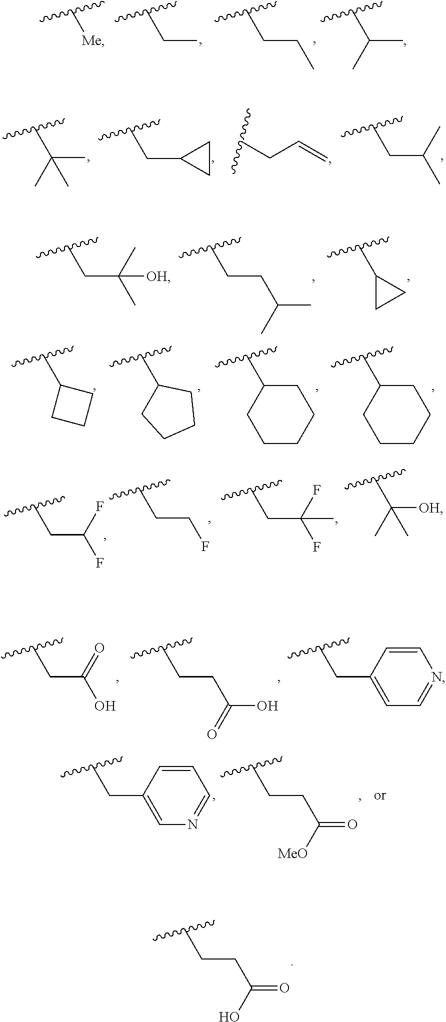

57-72. (canceled)

73. The method of claim 1, wherein D₁ is 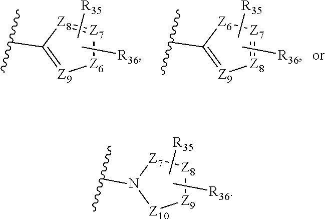
Wherein:
Z₆ is O, S, NR₄₀, or CHR₃₇;
Z₇, Z₈, Z₉ and Z₁₀ are independently N or CR₄₁;
R₃₅, R₃₆, R₃₇, R₄₀, and R₄₁ are each independently H, OH, NH₂, cycle, aryl, branched or unbranched alkyl alcohol, halo, branched or unbranched alkyl, amide, cyano, alkoxy, haloalkyl, aklylsulfonyl, nitrite, or alkylsulfanyl; or R₃₅ and R₃₆ together form an aryl or cycle that is attached to one or more of the atoms of D₁.

74-90. (canceled)

91. The method of claim 1, wherein formula I has a formula of

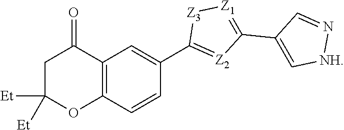

92-94. (canceled)

95. The method of claim 1, wherein the formula I has a formula of

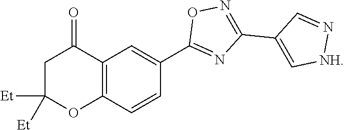 [SMILES: CCC1(CC)CC(=O)c2cc(-c3nc(-c4cn[nH]c4)no3)ccc2O1]

96-115. (canceled)

116. A method of treating or preventing a seizure, an epilepsy or an epilepsy-related syndrome in a subject in need thereof, the method comprising administering to the subject a compound having the formula of: 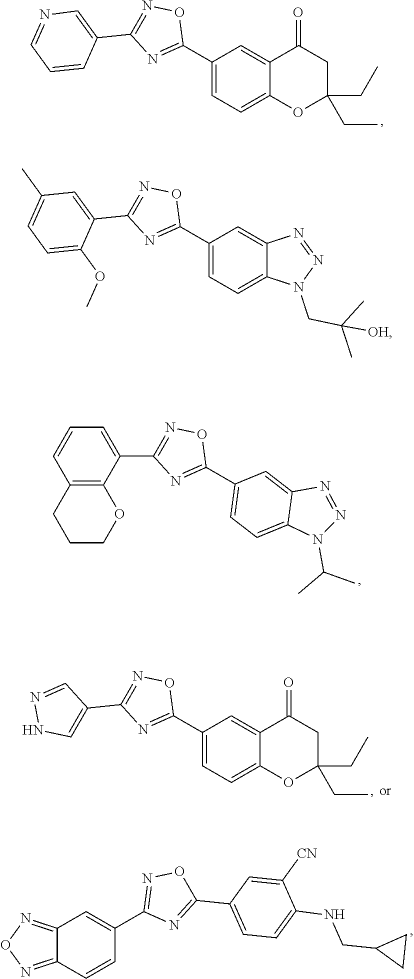
or a pharmaceutically acceptable salt thereof.

117. A method of treating or preventing a seizure or an epilepsy or an epilepsy-related syndrome in a subject in need thereof, the method comprising administering to the subject a pharmaceutical composition comprising a compound having Formula I or Formula II, or a pharmaceutically acceptable salt thereof: 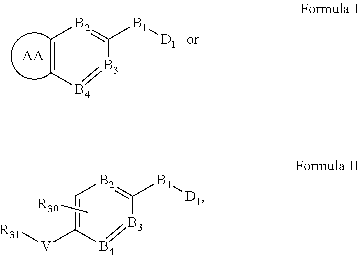
wherein:
AA is 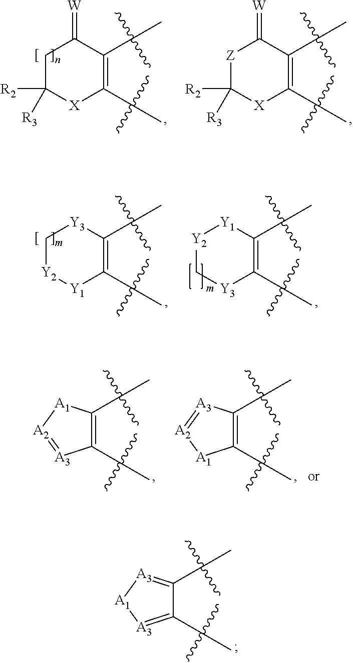
W is O, S, or NR₁;
X is O, S, or NR₄;
V is O, S, or NR₃₂;
Z is CHR₄₂ or NR₄₃;
n is 0, 1, 2, 3, or 4;
Y₁ and Y₂ are independently O, S, NR₅, C═O, C═S or C═NR₆;
Y₃ is O, S, CH₂, or NR₃₄;
m is 0, 1, 2, or 3;
A₁ is O, S, NR₇, C═O, or C═S;
A₂ and A₃ are independently CR₂₉ or N;
B₁ is an optionally substituted aryl or heteroaryl group, a carbocycle, or 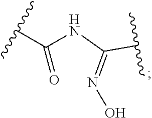 [SMILES: *C(=O)N/C(*)=N/O]
B₂, B₃, and B₄ are independently CR₃₈ or N;
D₁ is H, OH, NH₂, NO₂, cycle, optionally substituted aryl group, branched or unbranched alkyl alcohol, halo, branched or unbranched alkyl, amide, cyano, alkoxy, haloalkyl, aklylsulfonyl, nitrite, or alkylsulfanyl;
R₂ and R₃, are independently H, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₂ and R₃ are together optionally substituted cycloalkyl, or optionally substituted cycloheteroalkyl;
R₁, R₄, R₅, R₆, R₇, R₂₉, R₃₁, R₃₂, R₃₃, R₃₄, R₃₅, and R₄₃ are independently H, OH, NH₂, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl;
R₃₀ is independently H, CN, CF₃, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or optionally substituted haloalkyl; and
R₄₂ is independently Br, Cl, F, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl.

118. The method of claim 117, wherein the pharmaceutical composition further comprises a pharmaceutically acceptable carrier.

119. (canceled)

120. The method of claim 1, wherein the epilepsy is an intractable epilepsy.

121. The method of claim 120, wherein the intractable epilepsy is localization-related epilepsy, generalized epilepsy or syndromes thereof.

122. The method of claim 121, wherein the localization-related epilepsy is cortical epilepsy or temporal lobe epilepsy.

123. The method of claim 122, wherein the cortical epilepsy is frontal lobe epilepsy, parietal lobe epilepsy, or occipital lobe epilepsy.

124. The method of claim 1, wherein the epilepsy-related syndrome is an epileptic seizure selected from an intractable localization-related epilepsy seizure, an intractable secondary generalized seizure, an intractable complex partial seizure, or an intractable status epilepticus.

125. (canceled)

126. The method of claim 1, wherein the method further comprises administering at least one anti-epilepsy drug other than a compound of Formula I or Formula II.

127. The method of any one of claim 126, wherein the at least one anti-epilepsy drug is selected from the group consisting of carbamazepine, clonazepam, eslicarbazepine, ethosuximide, felbamate, gabapentin, lacosamide, lamotrigine, levetiracetam, oxcarbazepine, phenobarbital, phenytoin, pregabalin, primidone, rufinamide, tiagabine, topiramate, vigabatrin, valproate (valproic acid), and zonisamide.

128-130. (canceled)

131. The method of claim 1, wherein the compound, the pharmaceutically acceptable salt thereof, is administered in a therapeutically effective amount.
